(19) United States
(12) Patent Application Publication
(10) Pub. No.: US 2017/0119907 A1
(43) Pub. Date: May 4, 2017

(54) Targeted Therapeutics

(71) Applicant: MEDIVATION TECHNOLOGIES, INC., San Franciso, CA (US)

(72) Inventors: David Hung, San Francisco, CA (US); Sarvajit Chakravarty, San Francisco, CA (US); Roopa Rai, San Francisco, CA (US); Sebastian Bernales, San Francisco, CA (US); Balaji Dashrath Sathe, Noida (IN); Gonzalo Ureta, San Francisco, CA (US); Emma McCullagh, San Francisco, CA (US)

(73) Assignee: Medivation Technologies, Inc., San Francisco, CA (US)

(21) Appl. No.: 15/114,766

(22) PCT Filed: Jan. 28, 2015
(86) PCT No.: PCT/US2015/013334

§ 371 Date: Jul. 27, 2016

(30) Foreign Application Priority Data
Jan. 28, 2014 (IN) 258/DEL/2014

(51) Int. Cl. A61K 49/00; C09B 17/02; A61K 35/28

(52) U.S. Cl. CPC A61K 49/0026; A61K 49/003; A61K 35/28; C09B 17/02

(57) ABSTRACT

This disclosure describes compositions and methods for delivering and localizing therapeutic agents to therapeutic targets. This disclosure also provides multivalent forms of cationic dyes (“cationic dye multimers”) and methods by which these compounds can be used to treat joint injuries.

### CROSS-REFERENCE TO RELATED APPLICATIONS

This application claims priority to and incorporates by reference in its entirety Indian Patent Application No. 258/DEL/2014 filed Jan. 28, 2014. Each reference cited in this disclosure is incorporated herein by reference in its entirety.

### TECHNICAL FIELD

The disclosed inventions are in the field of targeted therapeutics.

### BRIEF DESCRIPTION OF THE FIGURES

FIG. 1. Schematic illustrating use of cationic dye multimers to anchor repair cells to an injured joint.

FIGS. 2A-B. Photomicrographs demonstrating binding of compounds to cells. FIG. 2A, binding of Compound 4 to ARH-77 cells (see Example B1a). FIG. 2B, binding of Compounds 4 and 20 to Raji cells (see Example B1b).

FIGS. 3A-C. Graphs of results from experiments demonstrating that Compounds 4 and 20 do not affect viability or proliferation of human MSCs or Raji cells at concentrations of 20 μM or below (Example B2). FIG. 3A, graph demonstrating proliferation of human MSCs in the presence of varying concentrations of Compound 4. FIG. 3B, graph comparing viability of human MSCs treated with varying concentrations of Compound 4 relative to viability of untreated human MSCs. FIG. 3C, graph demonstrating viability of Raji cells treated with varying concentrations of Compound 20 relative to viability of untreated control Raji cells.

FIGS. 4A-B. Photomicrographs demonstrating that Compound 4 binds to and stains human MSCs (FIG. 4A) and that Compounds 4 and 20 bind to and stain Raji cells (FIG. 4B).

FIGS. 5A-B. Photomicrographs demonstrating that Compound 4 (FIG. 5A) and Compounds 4 and 20 (FIG. 5B) bind to and stain rabbit dorsal femoral condyle explants.

FIGS. 6A-B. Photomicrographs demonstrating that compounds promote adherence of cells to rabbit dorsal femoral condyle explants. FIG. 6A, Compounds 4 and 20 promote adherence of human MSCs. FIG. 6B, Compounds 4 and 20 promote adherence of Raji cells.

FIG. 7. Photographs showing the effect of Compound 4-incubated MSCs in reducing cartilage damage induced by MIA.

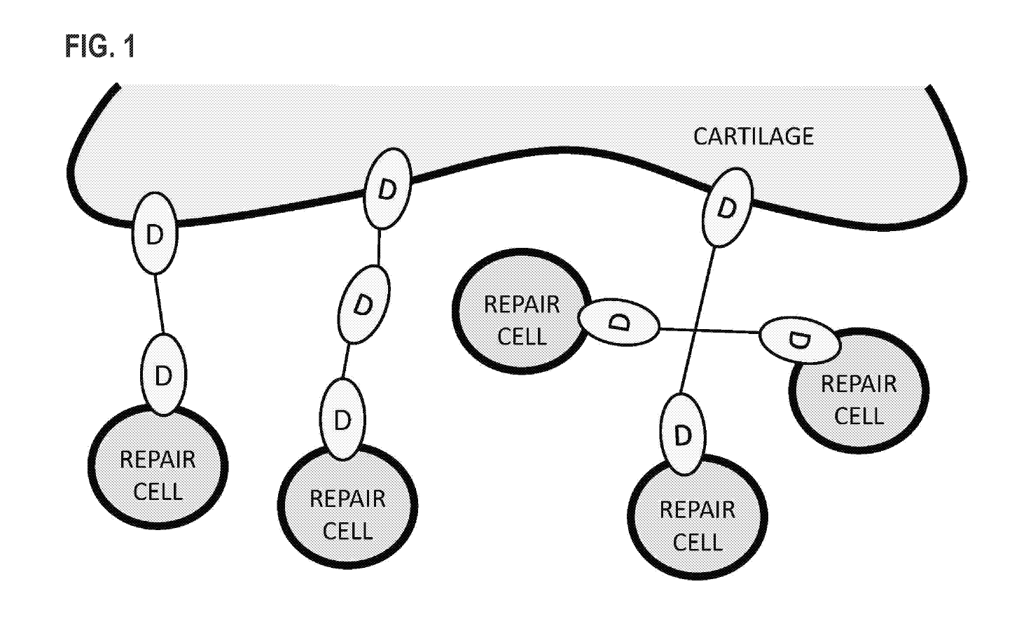

### DETAILED DESCRIPTION

This disclosure describes compositions and methods for delivering and localizing therapeutic agents to therapeutic targets which comprise cells with negatively charged cell membranes. A “negatively charged” cell membrane is a cell membrane to which a cationic dye will associate via electrostatic attraction. A conjugate comprising at least one cationic dye moiety (e.g., safranin-O, toluidine blue, azure A, azure B, azure C, acridine orange, acriflavine, methylene blue) which is bound to at least one therapeutic agent can be delivered to a therapeutic target and remain localized at the therapeutic target via association with the available cationic dye moiety portion of the conjugate.

“Binding” of the at least one cationic dye moiety to the at least one therapeutic agent may be by covalent or non-covalent attachment (i.e., via ionic or electrostatic attachment). In some embodiments, a conjugate comprises a cationic dye moiety which is covalently bound to a therapeutic agent, optionally via a linker such as linkers (a), (a.1), (a.2), (b), (b.1), (c), (c.1), (c.2), (d), (e), (e.1), (f), (f.1), (f.2), (g), (g.1), (g.2), (h), (h.1), (h.2), (i), (i.1), (i.2), (j), (j.1), (j.2), (k), (l), (l.1), (l.2), (m), (m.1), (n), (n.1), (n.2), (o), (p), (q), (r), and (s), described below. In other embodiments, a conjugate comprises at least one cationic dye moiety which is ionically bound to a therapeutic agent. In some embodiments, the cationic dye moiety portion of the conjugate is present as a cationic dye multimer (described below); in such embodiments, the conjugate may include two or more therapeutic agents, which may be the same or different, and which need not both be bound to the cationic dye multimer by the same type of binding. For example, a conjugate can comprise a cationic dye multimer which is covalently bound to one therapeutic agent and which is non-covalently bound to a second therapeutic agent.

Examples of cells with negatively charged cell membranes include, but are not limited to, endothelial cells of the digestive tract and lung, liver cells, cells of infectious organisms (e.g., bacteria), tumor cells, blood cells, myoblasts, and vascular endothelial cells.

Examples of therapeutic agents include, but are not limited to, small molecules (e.g., anti-inflammatories, cancer chemotherapeutics), nucleic acids (e.g., ribozymes, oligonucleotides, antisense RNA, siRNA, gene delivery vehicles), antibodies, and cells (e.g., repair cells, such as mesenchymal stem cells, described in more detail below). In some embodiments, a therapeutic agent may be contained in a delivery vehicle such as a liposome, nanoparticle.

In non-limiting embodiments, described below, in which the cationic dye moiety in the conjugate is present as a cationic dye multimer, the therapeutic agent is a repair cell.

### Use of Cationic Dye Multimers and Repair Cells to Treat Joint Injuries

In some embodiments, the disclosed conjugates comprise multivalent forms of cationic dyes (“cationic dye multimers”). Cationic dye multimers are, described in more detail below, together with methods by which conjugates comprising a cationic dye multimer can be used in conjunction with repair cells to treat joint injuries. Because they are multivalent, cationic dye multimers bind both to cartilage in an injured joint as well as to repair cells which can differentiate into new tissue (e.g., cartilage, tendon, meniscus), thereby stabilizing the joint and reducing pain. In addition to anchoring repair cells at an injured joint, cationic dye multimers permit visualization of the repair cells or the cartilage at the injury site, which facilitates the repair procedure.

A “repair cell” as used herein is a cell which, when exposed to appropriate conditions, differentiates into a cell which produces and secretes components needed to repair an injury to a joint (e.g., hyaline cartilage, tendon, meniscus). In some variations of the disclosed methods, a repair cell is a chondrocyte. In some variations of the disclosed methods, a repair cell is a mesenchymal stem cell (MSC). Methods of obtaining, culturing, and expanding populations of such repair cells are well known in the art. See, e.g., US 2004/0009157; US 2012/0148548; U.S. Pat. No. 5,486,359; and U.S. Pat. No. 5,226,914. In another variation, an active pharmaceutical ingredient could be envisioned in lieu of a repair cell.

In some variations of the disclosed methods, cationic dye multimers are applied directly to an injured joint, typically in a pharmaceutical composition for delivery to the injured joint. Such compositions typically are formulations suitable for intra-articular injection and may include one or more components such as chitin, chitosan, hyaluronan, chemically modified hyaluronan, saline, phosphate buffered saline, chondroitin sulfate, glucosamine, mannosamine, proteoglycan, proteoglycan fragments, or other polysaccharides or polymers. The cationic dye multimers to be applied may be all the same type or may be a mixture of types. Once the cationic dye multimers adhere to cartilage in the injured joint, repair cells are delivered to the site, where they are bound by the cationic dye multimers, thereby anchoring the repair cells in the appropriate place in the injured joint. In some variations, additional cofactors such as, for example, transforming growth factors (e.g. TGFβ), could be utilized either in co-application with the multimers described herein, or preceding that application, to stimulate extracellular matrix production and down-regulate matrix-degrading enzymes.

In other variations of the disclosed methods, cationic dye multimers, either all of the same type or a mixture of cationic dye multimer types, are bound to repair cells ex vivo, then the repair cells bearing the cationic dye multimers are delivered to the injured joint, either as a cell suspension or a sheet of cells, where they are anchored by the binding of the cationic dye multimers to cartilage in the injured joint. Binding of cationic dye multimers to the surface of repair cells can be carried out by any method known in the art. See, e.g., Sarkar et al., “Cell Surface Engineering of Mesenchymal Stem Cells,” in Vemuri et al., eds., Mesenchymal Stem Cell Assays and Applications, Methods in Molecular biology, chapter 35, pp. 505-23, 2011.

The disclosed methods can be carried out during an arthroscopic or open joint procedure and can be used to injuries at joints such as the acromioclavicular, carpometacarpal (finger or thumb), coracoclavicular, humeroulnar, humeroradial, radioulnar (distal, intermedial, proximal), intermetacarpal, interphalangeal, metacarpophalangeal, midcarpal, radiocarpal, shoulder, sternoclavicular, wrist, temporomandibular, sternocostal, xiphisternal, lumbosacral, sacroiliac, talocrural (ankle), hip, metatarsophalangeal, tarsometatarsal, tibiofemoral (knee) joints, and zygapophyseal joints.

Types of joint injuries which can be treated include damage to cartilage at a synovial joint occurring as a result of mechanical destruction due to trauma or progressive degeneration (osteoarthrosis; wear and tear) or associated with a disease or disorder, such as osteoarthritis, rheumatoid arthritis, gout, reactive arthritis, psoriatic arthritis, or juvenile arthritis. Other joint injuries include damage to tendons, ligaments, and the meniscus. Use of tissue engineering, including stem cell therapy, to treat such injuries has been reviewed. See, e.g. Nesic, et al. “Cartilage Tissue Engineering for Degenerative Joint Disease,” Advanced Drug Delivery Reviews (2006), 58(2):300-322; Johnstone, et al. “Tissue Engineering for Articular Cartilage Repair—The State of the Art,” European Cells and Materials (2013) 25:248-267; Dragoo, et al. “Healing Full-Thickness Cartilage Defects using Adipose-Derived Stem Cells,” Tissue Engineering (2007), 13(7):1615-1621. Use of animal models including chondral/osteochondral defects has been discussed recently. See, e.g. Cook, et al. “Animal Models of Cartilage Repair,” Bone Joint Res. (2014), 4:89-94. Bone-marrow derived MSCs, as well as human embryonic stem cells, have been shown to enhance cartilage repair in chondral defect models: Frisbie, et al. “Evaluation of Intra-Articular Mesenchymal Stem Cells to Augment Healing of Microfractures Chondral Defects,” Arthroscopy (2011), 27(11):1552-1561; Tsaiwei, et al. “Repair of Cartilage Defects in Arthritic Tissue with Differentiated Human Embryonic Stem Cells,” Tissue Engineering Part A (2014) 20(3-4):683-692.

In any of these methods, the repair cells and/or cartilage in the injured joint can be visualized using properties of the cationic dyes in the multimers. The extent of defects and repair to cartilage can be assessed and scored using methods known in the art such as, for example, the Histological-Histochemical Grading System (HHGS), the Osteoarthritis Research Society International (OARSI) Osteoarthritis Cartilage Histopathology Assessment System, the O'Driscoll score, the International Cartilage Repair Society (ICRS) II score, and the ‘Bern’ score. See, e.g. Rutgers, et al. “Evaluation of histological scoring systems for tissue-engineered, repaired and osteoarthritic cartilage”, Osteoarthritis and Cartilage (2010), 18:12-23; Grogan, et al. “Visual Histological Grading System for the Evaluation of in-vitro Generated Neocartilage”, Tissue Engineering (2006), 12(8):2141-2149.

### DEFINITIONS

“Aryl” refers to an unsaturated aromatic carbocyclic group having a single ring (e.g., phenyl) or multiple condensed rings (e.g., naphthyl or anthryl) which condensed rings may or may not be aromatic. In one variation, the aryl group contains from 6 to 14 annular carbon atoms (e.g., 6-14, 6-13, 6-12, 6-11, 6-10, 6-9, 6-8, 6-7, 7-14, 7-13, 7-12, 7-11, 7-10, 7-9, 7-8, 8-14, 8-13, 8-12, 8-11, 8-10, 8-9, 9-14, 9-13, 9-12, 9-11, 9-10, 10-14, 10-13, 10-12, 10-11, 11-14, 11-13, 11-12, 12-14, 12-13, 13-14, 6, 7, 8, 9, 10, 11, 12, 13, or 14). An aryl group having more than one ring where at least one ring is non-aromatic may be connected to the parent structure at either an aromatic ring position or at a non-aromatic ring position. In one variation, an aryl group having more than one ring where at least one ring is non-aromatic is connected to the parent structure at an aromatic ring position.

“Heteroaryl” refers to an unsaturated aromatic carbocyclic group having from 2 to 10 annular carbon atoms (e.g., 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, 9-10, 2, 3, 4, 5, 6, 7, 8, 9, or 10) and at least one annular heteroatom, including but not limited to heteroatoms such as nitrogen, oxygen and sulfur. A heteroaryl group may have a single ring (e.g., pyridyl, furyl) or multiple condensed rings (e.g., indolizinyl, benzothienyl) which condensed rings may or may not be aromatic. A heteroaryl group having more than one ring where at least one ring is non-aromatic may be connected to the parent structure at either an aromatic ring position or at a non-aromatic ring position. In one variation, a heteroaryl group having more than one ring where at least one ring is non-aromatic is connected to the parent structure at an aromatic ring position.

“Cycloalkyl” is a saturated cyclic hydrocarbon structure and can consist of one ring, such as cyclohexyl, or multiple rings, such as adamantyl. A cycloalkyl comprising more than one ring may be fused, spiro or bridged, or combinations thereof. A cycloalkyl can be a saturated cyclic hydrocarbon having from 3 to 13 annular carbon atoms (e.g., 3-13, 3-12, 3-11, 3-10, 3-9, 3-8, 3-7, 3-6, 3-5, 3-4, 4-13, 4-12, 4-11, 4-10, 4-9, 4-8, 4-7, 4-6, 4-5, 5-13, 5-12, 5-11, 5-10, 5-9, 5-8, 5-7, 5-6, 6-13, 6-12, 6-11, 6-10, 6-9, 6-8, 6-7, 7-13, 7-12, 7-11, 7-10, 7-9, 7-8, 8-13, 8-12, 8-11, 8-10, 8-9, 9-13, 9-12, 9-11, 9-10, 10-13, 10-12, 10-11, 11-13, 11-12, 12-13, 3, 4, 5, 6, 7, 8, 9, 10, 11, 12, or 13 annular carbon atoms). Examples of cycloalkyl groups include adamantyl, decahydronaphthalenyl, cyclopropyl, cyclobutyl, cyclopentyl, and cyclohexyl.

“Heterocyclyl” refers to a saturated or an unsaturated non-aromatic group having a single ring or multiple condensed rings, and having from 1 to 10 annular carbon atoms (e.g., 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, 9-10, 1, 2, 3, 4, 5, 6, 7, 8, 9, or 10) and from 1 to 4 annular heteroatoms (e.g., 1-4, 1-3, 1-2, 2-4, 2-3, 3-4, 1, 2, 3, o4 5), such as nitrogen, sulfur or oxygen. A heterocycle comprising more than one ring may be fused, spiro or bridged, or any combination thereof. In fused ring systems, one or more of the rings can be aryl or heteroaryl. A heterocycle having more than one ring where at least one ring is aromatic may be connected to the parent structure at either a non-aromatic ring position or at an aromatic ring position. In one variation, a heterocycle having more than one ring where at least one ring is aromatic is connected to the parent structure at a non-aromatic ring position.

Compounds according to the invention are detailed herein and in the appended claims. The invention includes the use of all of the compounds described herein, including any and all stereoisomers, including geometric isomers (cis/trans), salts (including pharmaceutically acceptable salts) and solvates of the compounds described herein, as well as methods of making such compounds.

The compounds depicted herein, by virtue of their cationic nature, may be present as salts even if salts are not depicted and it is understood that the invention embraces all salts and solvates of the compounds depicted here, as well as any non-salt or non-solvate form of the compound, as is well understood by the skilled artisan. In some embodiments, the salts of the compounds of the invention are pharmaceutically acceptable salts.

Where tautomeric forms may be present for any of the compounds described herein, each and every tautomeric form is intended even though only one or some of the tautomeric forms may be explicitly depicted. The tautomeric forms specifically depicted may or may not be the predominant forms in solution or when used according to the methods described herein.

### Cationic Dye Multimers

Cationic dye multimers can be linear, branched, or cyclic. In some variations, a cationic dye multimer is a dimer, in which two cationic dye moieties are linked with a linker as described below. In other variations, a cationic dye multimer is a trimer or higher order multimer containing, e.g., 3, 4, or 5 cationic dye moieties joined in various configurations by linkers such that the multimer is linear, branched, or cyclic. The cationic dye moieties in a multimer, as well as the linkers, can be the same or different, in various combinations, as set forth in the description below. In some embodiments, the binding capacity of a cationic dye multimer can be tuned based on the polarity/electron density of the charged multimer system such that, for example, the cationic dye multimer exhibits differential binding affinities to, e.g., cartilage and MSCs.

The linker moieties comprise a multivalent, rigid or non-rigid, alkyl chain containing appropriate functionality at the termini to bond with the cationic dye moieties, as also set forth in the description below. Such linkers could, for example, comprise a bivalent chain thus having a cationic dye at each end resulting in a dimer. Other combinations and configurations are similarly described herein.

### Cationic Dyes

In some variations, cationic dyes which can be used to make cationic dye multimers as described herein have a planar tri-aromatic core with the potential to have a positive charge at physiological pH. Representative examples of such cationic dyes are shown below, with the “wiggle line” indicating one possible point of attachment to a linker to a dimer or higher oligomer:

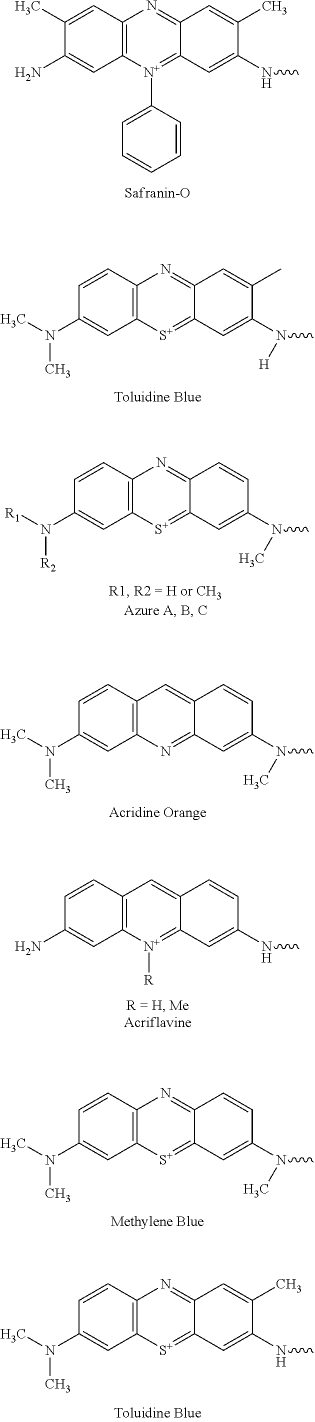

### Linkers

The amino groups on cationic dyes such as safranin-O, toluidine blue, azure A, azure B, azure C, acridine orange, acriflavine, and methylene blue are unreactive. These amino groups can however be functionalized through reactions that provide “handles” which comprise a carboxylic acid or an amine; cationic dyes comprising such handles are referred to herein as “cationic dye moieties.”

As provided in the Examples herein, synthetic routes to dyes presented herein can result in positional isomeric products, for example those shown below for Safranin:

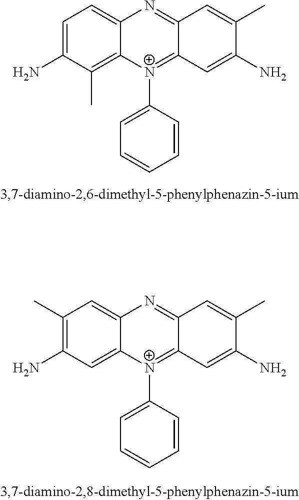 [SMILES: Cc1cc2nc3ccc(N)c(C)c3[n+](-c3ccccc3)c2cc1N ; Cc1cc2nc3cc(C)c(N)cc3[n+](-c3ccccc3)c2cc1N]

wherein as presented here a methyl group can be at the 6- or 8-position. Commercially available sources of such reagents can comprise a mixture of such regioisomers. All compounds of the invention presented herein encompass any and all derivatives from such regioisomeric dyes.

Cationic dye moieties can be functionalized with the appropriately substituted linkers described below using reactions known to those skilled in the art; this is illustrated for safranin-O in the schematic below:

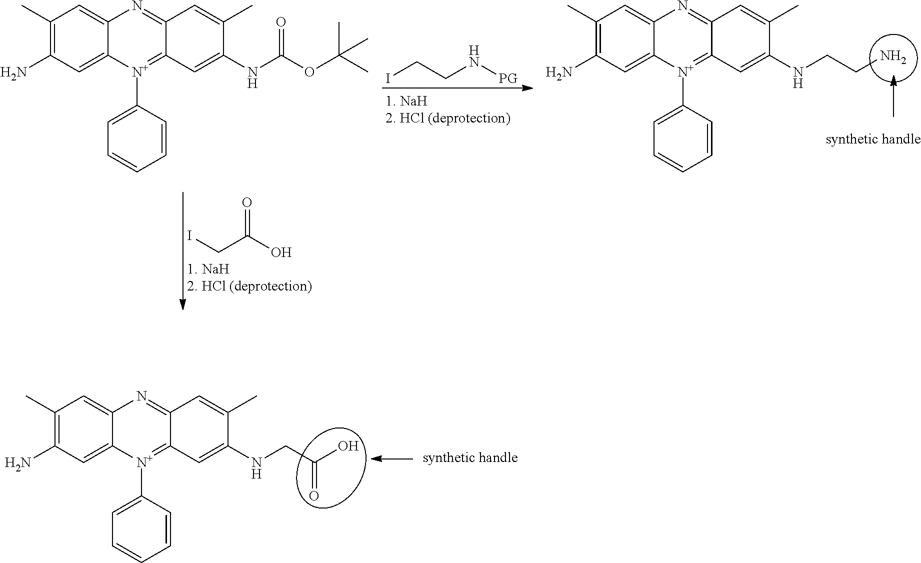

For example, compounds 14, 15, 16, 17, 18, and 19 in Table 1, below, can be synthesized using terephthalic acid (CAS #100-21-0), 2,5-pyridinedicarboxylic acid (CAS#100-26-5), 4,5-imidazoledicarboxylic acid (CAS#570-22-9), 2-(ethoxycarbonyl)-1,3-thiazole-4-carboxylic acid (CAS#911466-96-1), 1,4-cyclohexanedicarboxylic acid (CAS#1076-97-7), and 4-oxo-cyclopentane-1,2-dicarboxylic acid diethyl ester (CAS#914637-96-0), respectively, as a reagent.

In some cationic dye multimers in which at least one of the cationic dye multimers is safranin-O, the pendant phenyl ring of the safranin-O is unsubstituted. In some cationic dye multimers in which at least one of the cationic dye multimers is safranin-O, the pendant phenyl ring of the safranin-O is substituted with 1-3 (e.g., 1-3, 1-2, 1, 2, or 3) electron-donating or electron-withdrawing groups, which may be at any available position on the pendant phenyl ring. Examples of electron-donating groups include —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). Examples of electron-withdrawing groups include —NO₂, —NR₃⁺, halo (e.g., F, Br, Cl, I), trihalides (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations, linkers comprise a positive charge, which can be provided by a positive charged substituent such as an amino alkyl, amino heterocyclyl, or N-containing heteroaromatic group. In some variations, positively charged linkers comprise amino acids such as Lys, Arg, or His. If a rigid linker is desired, one or more aromatic rings, cycloalkyl rings, heteroaromatic rings, or heterocyclyl rings, can be used to provide rigidity. Rigidity can also be increased by restricting rotation of the linker through use of sp- or sp²-hybridized carbon atoms in a chain, for example with double- or triple-bonds, keto groups, and the like, as well as by employing bulky side-chains such as, for example, gem-dialkyl groups. Linkers which can be used in cationic dye multimers include linkers (a), (a.1), (a.2), (b), (b.1), (c), (c.1), (c.2), (d), (e), (e.1), (f), (f.1), (f.2), (g), (g.1), (g.2), (h), (h.1), (h.2), (i), (i.1), (i.2), (j), (j.1), (j.2), (k), (l), (l.1), (l.2), (m), (m.1), (n), (n.1), (n.2), (o), (p), (q), (r), and (s), below:

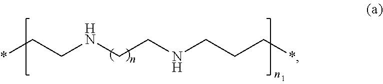

in which n is 1-6, n₁ is 1-4, and each * is an attachment site for a cationic dye moiety;

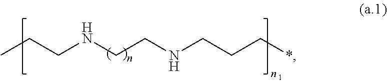

in which n is 1-6, n₁ is 1-4, and * is an attachment site for a cationic dye moiety;

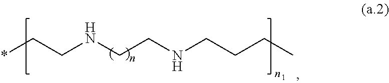

in which n is 1-6, n₁ is 1-4, and * is an attachment site for a cationic dye moiety;

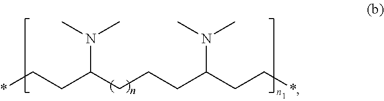

in which n is 0-6, n₁ is 1-4, and each * is an attachment site for a cationic dye moiety;

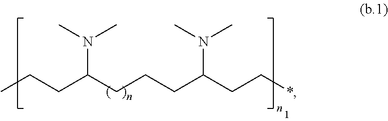

in which n is 0-6, n₁ is 1-4, and * is an attachment site for a cationic dye moiety;

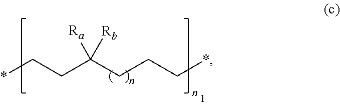

in which n is 0-6; n₁ is 1-4; for each independent instance of Rₐ and R_{b}, (1) Rₐ and R_{b} independently are H or CH₃, or (2) Rₐ and R_{b} are

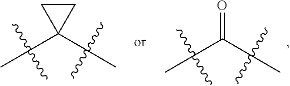

or (3) two of CRₐR_{b} are

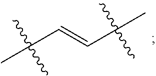 [SMILES: */C=C/*]

and each * is an attachment site for a cationic dye moiety. Examples of linker (c) include

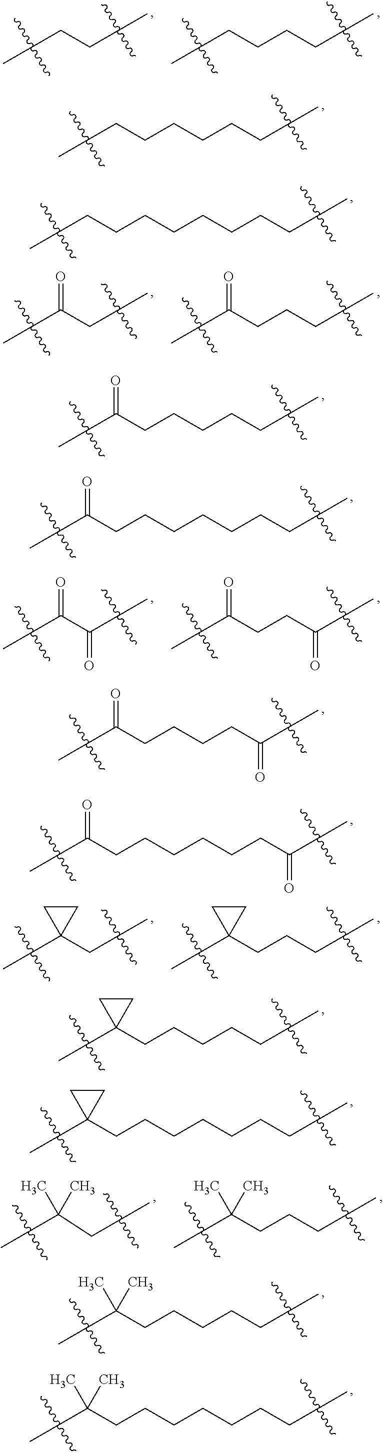

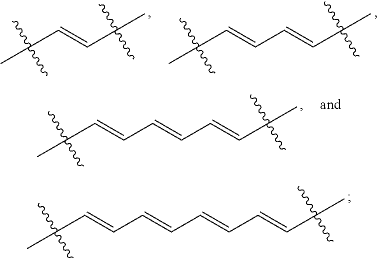

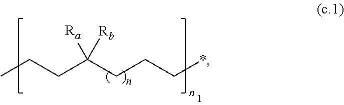

in which n is 0-6; n₁ is 1-4; for each independent instance of Rₐ and R_{b}, (1) Rₐ and R_{b} independently are H or CH₃ or (2) Rₐ and R_{b} are

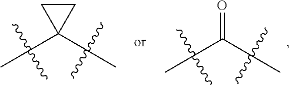

or (3) two of CRₐR_{b} are

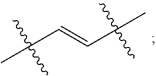 [SMILES: */C=C/*]

and each * is an attachment site for a cationic dye moiety;

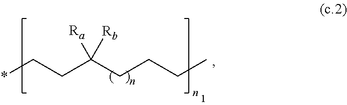

in which n is 0-6; n₁ is 1-4; for each independent instance of Rₐ and R_{b}, (1) Rₐ and R_{b} independently are H or CH₃ or (2) Rₐ and R_{b} are

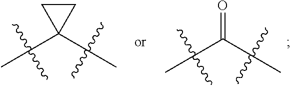

or (3) two of CRₐR_{b} are

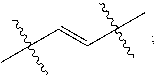 [SMILES: */C=C/*]

and * is an attachment site for a cationic dye moiety;

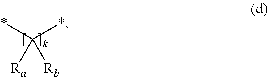

where k is 2-10; for each independent instance of Rₐ and R_{b}, Rₐ and R_{b} (1) independently are H or CH₃, or (2) Rₐ and R_{b} are

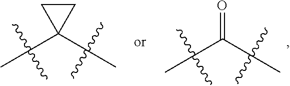

or (3) two of CRₐR_{b} are

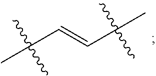 [SMILES: */C=C/*]

and each * is an attachment site for a cationic dye moiety;

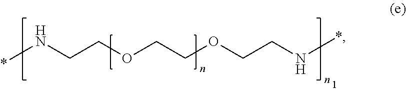

in which n is 0-6, n₁ is 1-4, and each * is an attachment site for a cationic dye moiety;

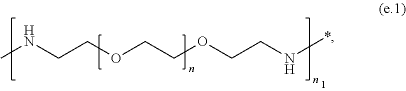

in which n is 0-6, n₁ is 1-4, and * is an attachment site for a cationic dye moiety;

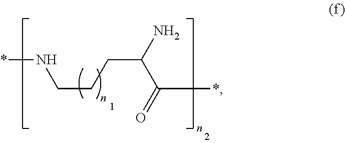

in which n₁ is 0-5, n₂ is 1-5 and each * is an attachment site for a cationic dye moiety;

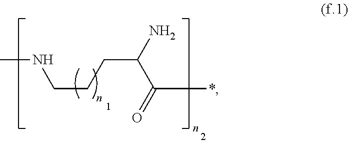

in which n₁ is 0-5, n₂ is 1-5 and * is an attachment site for a cationic dye moiety;

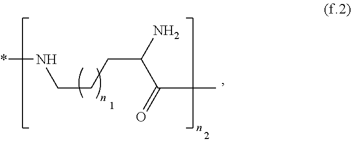

in which n₁ is 0-5, n₂ is 1-5 and * is an attachment site for a cationic dye moiety;

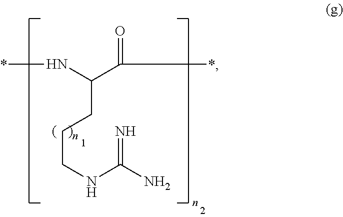

in which n₁ is 0-5, n₂ is 1-5 and each * is an attachment site for a cationic dye moiety;

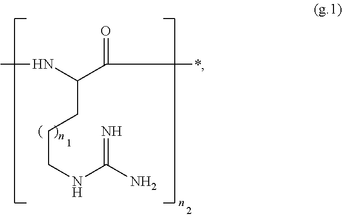

in which n₁ is 0-5, n₂ is 1-5 and * is an attachment site for a cationic dye moiety;

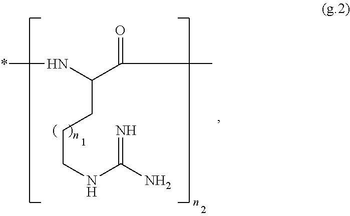

in which n₁ is 0-5, n₂ is 1-5 and * is an attachment site for a cationic dye moiety;

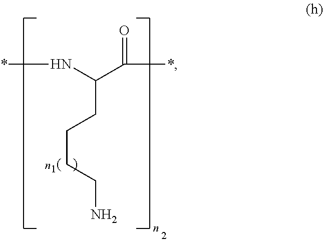

in which n₁ is 0-5, n₂ is 1-5, and each * is an attachment site for a cationic dye moiety;

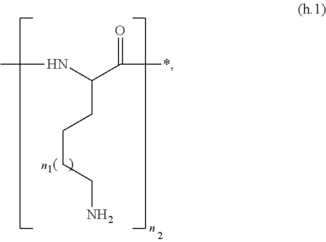

in which n₁ is 0-5, n₂ is 1-5, and * is an attachment site for a cationic dye moiety;

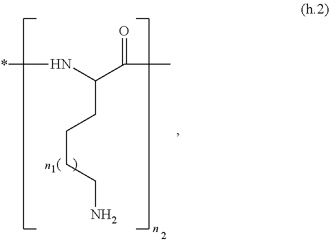

in which n₁ is 0-5, n₂ is 1-5, and * is an attachment site for a cationic dye moiety;

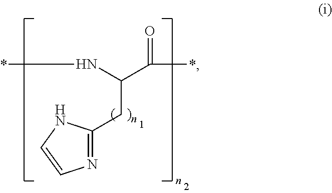

in which n₁ and n₂ independently are 1-5 and each * is an attachment site for a cationic dye moiety;

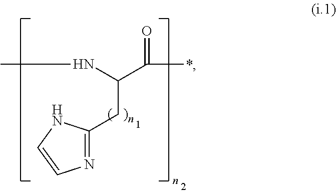

in which n₁ and n₂ independently are 1-5 and * is an attachment site for a cationic dye moiety;

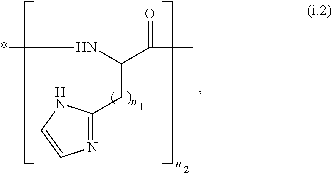

in which n₁ and n₂ independently are 1-5 and * is an attachment site for a cationic dye moiety;

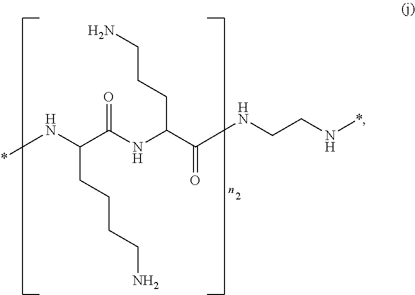

in which n₂ is 1-5 and each * is an attachment site for a cationic dye moiety;

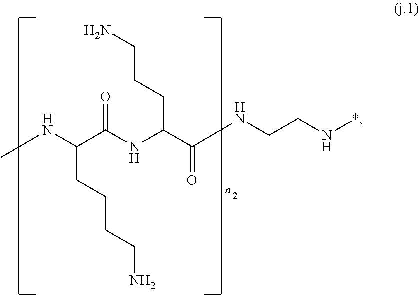

in which n₂ is 1-5 and * is an attachment site for a cationic dye moiety;

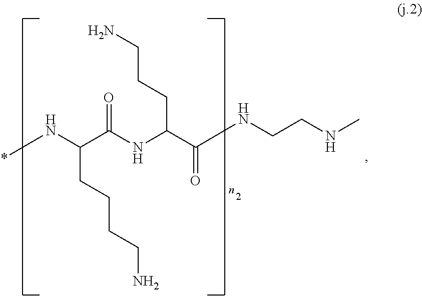

in which n₂ is 1-5 and * is an attachment site for a cationic dye moiety;

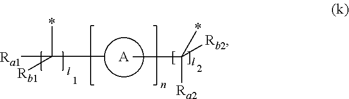

in which l₁ and l₂ independently are 1-4, n is 1-4; ring A is aryl, heteroaryl, cycloalkyl, or heterocyclyl; for each independent instance of Rₐ₁ and R_{b1}, Rₐ₁ and R_{b1} (1) independently are H or CH₃, or (2) Rₐ₁ and R_{b1} independently are

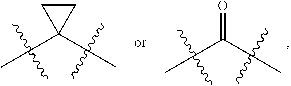

or (3) two of CRₐ₁R_{b1} are

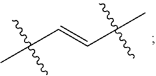 [SMILES: */C=C/*]

for each independent instance of Rₐ₂ and R_{b2}, Rₐ₂ and R_{b2} (1) independently are H or CH₃, or (2) Rₐ₂ and R_{b2} independently are

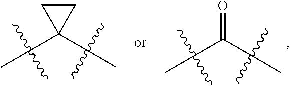

or (3) two of CRₐ₂R_{b2} are

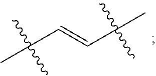 [SMILES: */C=C/*]

and each * is an attachment site for a cationic dye moiety;

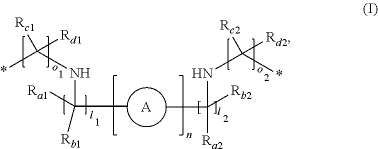

in which b, l₁, l₂, n, o₁ and o₂ independently are 1-4; ring A is aryl, heteroaryl, cycloalkyl, or heterocyclyl; for each independent instance of Rₐ₁ and R_{b1}, Rₐ₁ and R_{b1} (1) independently are H or CH₃, or (2) Rₐ₁ and R_{b1} independently are

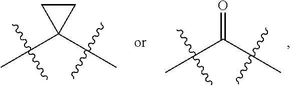

or (3) two of CRₐ₁R_{b1} are

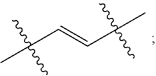 [SMILES: */C=C/*]

for each independent instance of Rₐ₂ and R_{b2}, Rₐ₂ and R_{b2} (1) independently are H or CH₃, or (2) Rₐ₂ and R_{b2} independently are

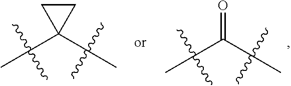

or (3) two of CRₐ₂R_{b2} are

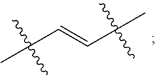 [SMILES: */C=C/*]

for each independent instance of R_{c1} and R_{d1}, R_{c1} and R_{d1} (1) independently are H or CH₃, or (2) R_{c1} and R_{d1} independently are

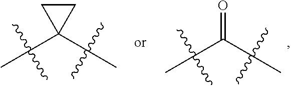

or (3) two of CR_{c1}R_{d1} are

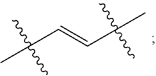 [SMILES: */C=C/*]

for each independent instance of R_{c2} and R_{d2}, R_{c2} and R_{d2} (1) independently are H or CH₃, or (2) R_{c2} and R_{d2} independently are

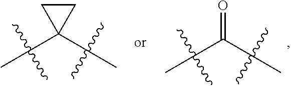

or (3) two of CR_{c2}R_{d2} are

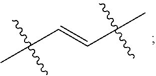 [SMILES: */C=C/*]

and each * is an attachment site for a cationic dye moiety;

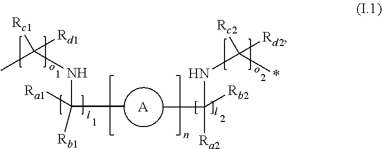

in which l₁, l₂, n, o₁, and o₂ independently are 1-4; ring A is aryl, heteroaryl, cycloalkyl, or heterocyclyl; for each independent instance of Rₐ₁ and R_{b1}, Rₐ₁ and R_{b1} (1) independently are H or CH₃, or (2) Rₐ₁ and R_{b1} independently are

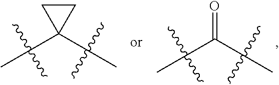

or (3) two of CRₐ₁R_{b1} are

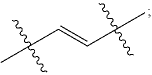 [SMILES: */C=C/*]

for each independent instance of Rₐ₂ and R_{b2}, Rₐ₂ and R_{b2} (1) independently are H or CH₃, or (2) Rₐ₂ and R_{b2} independently are

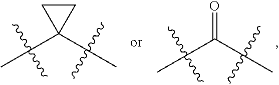

or (3) two of CRₐ₂R_{b2} are

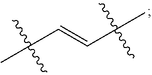 [SMILES: */C=C/*]

for each independent instance of R_{c1} and R_{d1}, R_{c1} and R_{d1} (1) independently are H or CH₃, or (2) R_{c1} and R_{d1} independently are

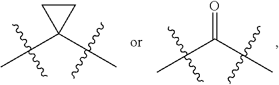

or (3) two of CR_{c1}R_{d1} are

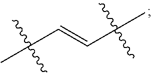 [SMILES: */C=C/*]

for each independent instance of R_{c2} and R_{d2}, R_{c2} and R_{d2} (1) independently are H or CH₃, or (2) R_{c2} and R_{d2} independently are

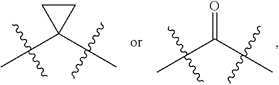

or (3) two of CR_{c2}R_{d2} are

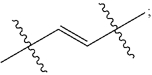 [SMILES: */C=C/*]

and * is an attachment site for a cationic dye moiety;

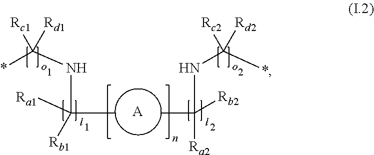

in which l₁, l₂, n, o₁, and o₂ independently are 1-4; ring A is aryl, heteroaryl, cycloalkyl, or heterocyclyl; for each independent instance of Rₐ₁ and R_{b1}, Rₐ₁ and R_{b1} (1) independently are H or CH₃, or (2) Rₐ₁ and R_{b1} independently are

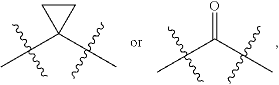

or (3) two of CRₐ₁R_{b1} are

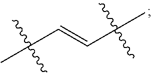 [SMILES: */C=C/*]

for each independent instance of Rₐ₂ and R_{b2}, Rₐ₂ and R_{b2} (1) independently are H or CH₃, or (2) Rₐ₂ and R_{b2} independently are

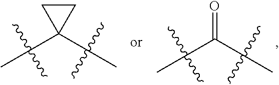

or (3) two of CRₐ₂R_{b2} are

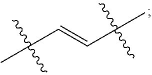 [SMILES: */C=C/*]

for each independent instance of R_{c1} and R_{d1}, R_{c1} and Rd₁ (1) independently are H or CH₃, or (2) R_{c1} and R_{d1} independently are

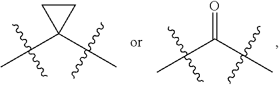

or (3) two of CR_{c1}R_{d1} are

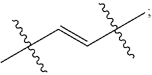 [SMILES: */C=C/*]

for each independent instance of R_{c2} and R_{d2}, R_{c2} and R_{d2} (1) independently are H or CH₃, or (2) R_{c2} and R_{d2} independently are

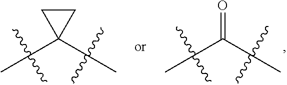

or (3) two of CR_{c2}R_{d2} are

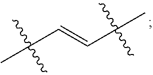 [SMILES: */C=C/*]

and * is an attachment site for a cationic dye moiety;

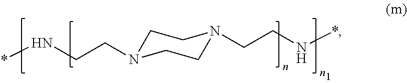

in which n is 0-6, n₁ is 1-4, and each * is an attachment site for a cationic dye moiety;

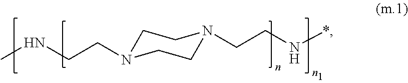

in which n is 0-6, n₁ is 1-4, and * is an attachment site for a cationic dye moiety;

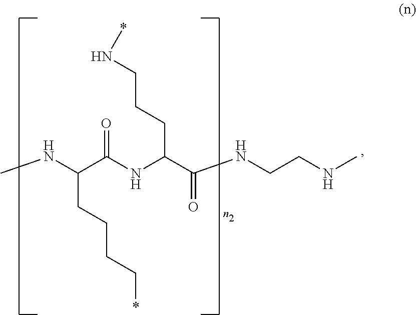

in which n₂ is 1-5 and each * is an attachment site for a cationic dye moiety;

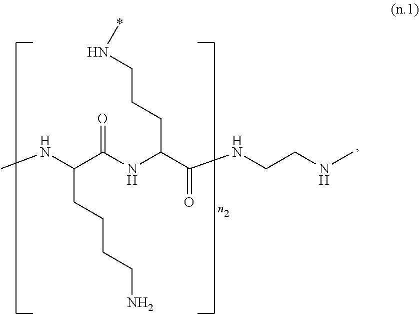

in which n₂ is 1-5 and * is an attachment site for a cationic dye moiety;

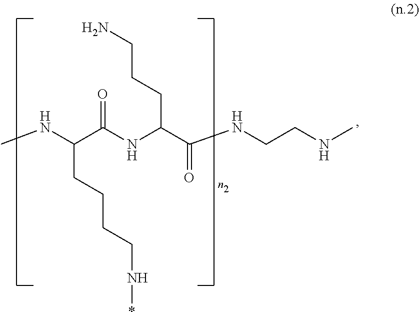

in which n₂ is 1-5 and * is an attachment site for a cationic dye moiety;

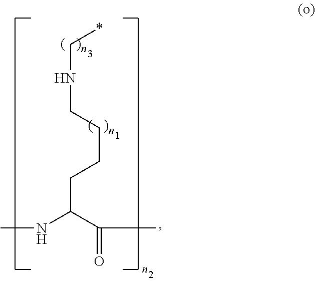

in which in which n₁ is 0-5, n₂ is 1-5, n3 is 0-5, and * is the attachment site for a cationic dye moiety;

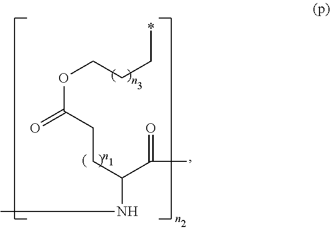

in which n₁ is 0-5, n₂ is 1-5, n3 is 0-5, and * is the attachment site for a cationic dye moiety;

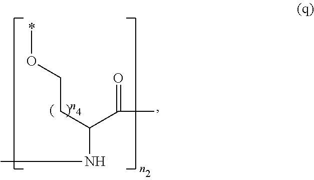

in which n₄ is 0-5, n₂ is 1-5, and * is an attachment site for a cationic dye moiety;

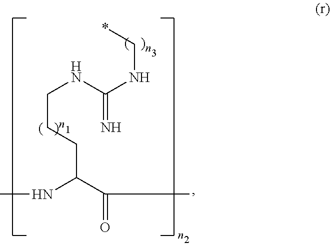

in which n₁ is 0-5, n₂ is 1-5, n3 is 0-5, and * is the attachment site for a cationic dye moiety; and

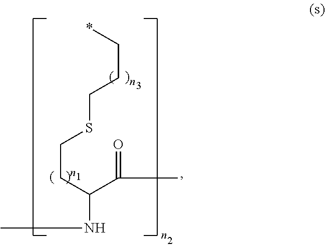

in which n₁ is 0-5, n₂ is 1-5, n₃ is 0-5 and * is the attachment site for a cationic dye moiety.

### Cationic Dyes Comprising Linkers

This disclosure also provides cationic dye moieties which comprise one or more linkers, which are suitable for preparing the conjugates and the cationic dimers disclosed herein. Cationic dyes useful for these embodiments include, but are not limited to, safranin-O, toluidine blue, azure A, azure B, azure C, acridine orange, acriflavine, and methylene blue. In some embodiments, the cationic dye moiety is present as a monomer. In other embodiments, the cationic dye moiety is present as a multimer. In either of these embodiments, the cationic dye moiety comprises one or more linkers, which may be the same or different. Suitable linkers include, but are not limited to, linkers (a), (a.1), (a.2), (b), (b.1), (c), (c.1), (c.2), (d), (e), (e.1), (f), (f.1), (f.2), (g), (g.1), (g.2), (h), (h.1), (h.2), (i), (i.1), (i.2), (j), (j.1), (j.2), (k), (l), (l.1), (l.2), (m), (m.1), (n), (n.1), (n.2), (o), (p), (q), (r), and (s), described above.

### Examples of Cationic Dye Dimers

Some cationic dye dimers fall within formula (1):

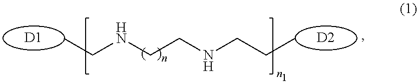

in which each of D1 and D2 is a cationic dye moiety, n is 1-6, and n₁ is 1-4. In some variations of formula (1), D1 and D2 are different cationic dye moieties. In other variations of formula (1), D1 and D2 are the same cationic dye moiety. In some variations of formula (1), D1 and D2 independently are selected from the group consisting of safranin-O, toluidine blue, azure A, azure B, azure C, acridine orange, acriflavine, and methylene blue.

In some variations of formula (1) described in the paragraphs above, n is 1-6, 1-5, 1-4, 1-3, 1-2, 2-6, 2-5, 2-4, 2-3, 3-6, 3-5, 3-4, 4-6, 4-5, 5-6, 1, 2, 3, 4, 5, or 6.

In some variations of formula (1) described in the paragraphs above, n₁ is 1-4, 1-3, 1-2, 2-4, 2-3, 3-4, 1, 2, 3, or 4.

In some variations of formula (1) described in the paragraphs above, D1 is safranin-O. In some variations of formula (1) described in the paragraphs above, D2 is safranin-O. In some variations of formula (1) described in the paragraphs above, D1 and D2 are safranin-O.

In some variations of formula (1) described in the paragraph above, the pendant phenyl ring of D1 is unsubstituted. In some variations of formula (1) described in the paragraph above, the pendant phenyl ring of D1 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D1 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D1 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (1) described in the paragraph above, the pendant phenyl ring of D2 is unsubstituted. In some variations of formula (1) described in the paragraph above, the pendant phenyl ring of D2 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D2 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D2 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

Some cationic dye dimers fall within formula (2):

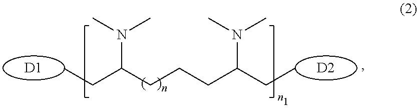

in which each of D1 and D2 is a cationic dye moiety, n is 0-6, and n₁ is 1-4.

In some variations of formula (2), D1 and D2 are different cationic dye moieties. In other variations of formula (2), D1 and D2 are the same cationic dye moiety. In some variations of formula (2), D1 and D2 are independently selected from the group consisting of safranin-O, toluidine blue, azure A, azure B, azure C, acridine orange, acriflavine, and methylene blue.

In some variations of formula (2) described in the paragraphs above, n is 0-6, 0-5, 0-4, 0-3, 0-2, 0-1, 1-6, 1-5, 1-4, 1-3, 1-2, 2-6, 2-5, 2-4, 2-3, 3-6, 3-5, 3-4, 4-6, 4-5, 5-6, 0, 1, 2, 3, 4, 5, or 6.

In some variations of formula (2) described in the paragraphs above, n₁ is 1-4, 1-3, 1-2, 2-4, 2-3, 3-4, 1, 2, 3, or 4.

In some variations of formula (2) described in the paragraphs above, D1 is safranin-O. In some variations of formula (2) described in the paragraphs above, D2 is safranin-O. In some variations of formula (2) described in the paragraphs above, D1 and D2 are safranin-O.

In some variations of formula (2) described in the paragraph above, the pendant phenyl ring of D1 is unsubstituted. In some variations of formula (2) described in the paragraph above, the pendant phenyl ring of D1 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D1 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D1 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (2) described in the paragraph above, the pendant phenyl ring of D2 is unsubstituted. In some variations of formula (2) described in the paragraph above, the pendant phenyl ring of D2 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D2 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D2 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

Some cationic dye dimers fall within formula (3):

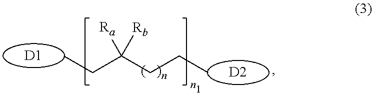

in which each of D1 and D2 is a cationic dye moiety; n is 0-6, and n₁ is 1-4; and, for each independent instance of Rₐ and R_{b}, (1) Rₐ and R_{b} independently are H or CH₃, or (2) Rₐ and R_{b} are

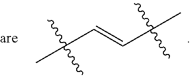 [SMILES: */C=C/*]

or (3) two of CRₐR_{b}

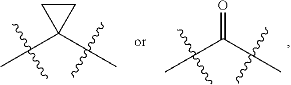

In some variations of formula (3), D1 and D2 are different cationic dye moieties. In other variations of formula (3), D1 and D2 are the same cationic dye moiety. In some variations of formula (3), D1 and D2 are independently selected from the group consisting of safranin-O, toluidine blue, azure A, azure B, azure C, acridine orange, acriflavine, and methylene blue.

In some variations of formula (3) described in the paragraphs above, n is 0-6, 0-5, 0-4, 0-3, 0-2, 0-1, 1-6, 1-5, 1-4, 1-3, 1-2, 2-6, 2-5, 2-4, 2-3, 3-6, 3-5, 3-4, 4-6, 4-5, 5-6, 0, 1, 2, 3, 4, 5, or 6.

In some variations of formula (3) described in the paragraphs above, n₁ is 1-4, 1-3, 1-2, 2-4, 2-3, 3-4, 1, 2, 3, or 4.

In some variations of formula (3) described in the paragraphs above in which n₁ is 1, Rₐ and R_{b} are both H. In some variations of formula (3) described in the paragraphs above in which n₁ is 1, Rₐ is H and R_{b} is CH₃. In some variations of formula (3) described in the paragraphs above in which n₁ is 1, Rₐ and R_{b} are both CH₃. In some variations of formula (3) described in the paragraphs above in which n₁ is 1, Rₐ and R_{b} are

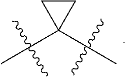 [SMILES: *C1(*)CC1]

In some variations of formula (3) described in the paragraphs above in which n₁ is 1, Rₐ and R_{b} are

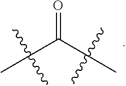 [SMILES: *C(*)=O]

In variations of formula (3) described in the paragraphs above in which n₁ is 2, the two instances of Rₐ and R_{b} are indicated as Rₐ₁ and R_{b1} and Rₐ₂ and R_{b2}, respectively. In some variations of formula (3) described in the paragraphs above in which n₁ is 2, each of Rₐ₁ and R_{b1} and Rₐ₂ and R_{b2} is H. In some variations of formula (3) described in the paragraphs above in which n₁ is 2, each of Rₐ₁ and R_{b1} and Rₐ₂ and R_{b2} is CH₃. In some variations of formula (3) described in the paragraphs above in which n₁ is 2, each of Rₐ₁ and Rₐ₂ is H and each of R_{b1} and R_{b2} is CH₃. In some variations of formula (3) described in the paragraphs above in which n₁ is 2, each of Rₐ₁, Rₐ₂, and R_{b1} is H and R_{b2} is CH₃.

In some variations of formula (3) described in the paragraphs above in which n₁ is 2, each of Rₐ₁ and R_{b1} is H and Rₐ₂ and R_{b2} are

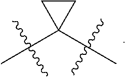 [SMILES: *C1(*)CC1]

In some variations of formula (3) described in the paragraphs above in which n₁ is 2, each of Rₐ₁ and R_{b1} is H and Rₐ₂ and R_{b2} are

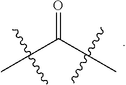 [SMILES: *C(*)=O]

In some variations of formula (3) described in the paragraphs above in which n₁ is 2, each of Rₐ₁ and R_{b1} is CH₃ and Rₐ₂ and R_{b2} are

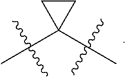 [SMILES: *C1(*)CC1]

In some variations of formula (3) described in the paragraphs above in which n₁ is 2, each of Rₐ₁ and R_{b1} is CH₃ and Rₐ₂ and R_{b2} are

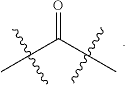 [SMILES: *C(*)=O]

In some variations of formula (3) described in the paragraphs above in which n₁ is 2, Rₐ₁ is H, R_{b1} is CH₃, and Rₐ₂ and R_{b2} are

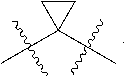 [SMILES: *C1(*)CC1]

In some variations of formula (3) described in the paragraphs above in which n₁ is 2, Rₐ₁ is H, R_{b1} is CH₃, and Rₐ₂ and R_{b2} are

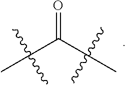 [SMILES: *C(*)=O]

In some variations of formula (3) described in the paragraphs above in which n₁ is 2, Rₐ₁ and R_{b1} and Rₐ₂ and R_{b2} together are

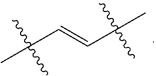 [SMILES: */C=C/*]

In some variations of formula (3) described in the paragraphs above in which n₁ is 2, Rₐ₁ and R_{b1} are

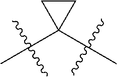 [SMILES: *C1(*)CC1]

Rₐ₂ and R_{b2} are

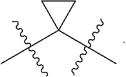 [SMILES: *C1(*)CC1]

In some variations of formula (3) described in the paragraphs above in which n₁ is 2, Rₐ₁ and R_{b1} are

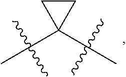 [SMILES: *C1(*)CC1]

and Rₐ₂ and R_{b2} are

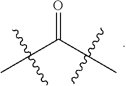 [SMILES: *C(*)=O]

In some variations of formula (3) described in the paragraphs above in which n₁ is 2, Rₐ₁ and R_{b1} are

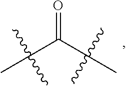 [SMILES: *C(*)=O]

and Rₐ₂ and R_{b2} are

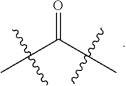 [SMILES: *C(*)=O]

In variations of formula (3) described in the paragraphs above in which n₁ is 3, the three instances of Rₐ and R_{b} are indicated as Rₐ₁ and R_{b1}; Rₐ₂ and R_{b2}; and Rₐ₁ and R_{b3}, respectively. In some variations of formula (3) described in the paragraphs above in which n₁ is 3, In some variations of formula (3) described in the paragraphs above in which n₁ is 3, each of Rₐ₁, R_{b1}, Rₐ₂, R_{b2}, Rₐ₃, and R_{b3} is H. In some variations of formula (3) described in the paragraphs above in which n₁ is 3, each of Rₐ₁, R_{b1}, Rₐ₂, R_{b2}, Rₐ₃, and R_{b3} is CH₃. In some variations of formula (3) described in the paragraphs above in which n₁ is 3, each of Rₐ₁, R_{b1}, Rₐ₂, and R_{b2}, is H and each of Rₐ₃ and R_{b3} is CH₃. In some variations of formula (3) described in the paragraphs above in which n₁ is 3, each of Rₐ₁, R_{b1}, Rₐ₂, and R_{b2}, is CH₃ and each of Rₐ₃ and R_{b3} is H. In some variations of formula (3) described in the paragraphs above in which n₁ is 3, Rₐ₁ is H and each of Rₐ₂, Rₐ₃, R_{b1}, R_{b2}, and R_{b3} is CH₃. In some variations of formula (3) described in the paragraphs above in which n₁ is 3, each of Rₐ₁ and Rₐ₂ is H and each of Rₐ₃, R_{b1}, R_{b2}, and R_{b3} is CH₃. In some variations of formula (3) described in the paragraphs above in which n₁ is 3, each of Rₐ₁, Rₐ₂, and Rₐ₃ is H and each of R_{b1}, R_{b2}, an R_{b3} is CH₃.

In some variations of formula (3) described in the paragraphs above in which n₁ is 3, each of Rₐ₁, R_{b1}, Rₐ₂, and R_{b2}, is H and Rₐ₃ and R_{b3} are

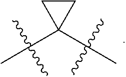 [SMILES: *C1(*)CC1]

In some variations of formula (3) described in the paragraphs above in which n₁ is 3, each of Rₐ₁, R_{b1}, Rₐ₂, and R_{b2}, is CH₃ and Rₐ₃ and R_{b3} are

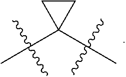 [SMILES: *C1(*)CC1]

In some variations of formula (3) described in the paragraphs above in which n₁ is 3, Rₐ₁ is H and each of Rₐ₂, R_{b1}, and R_{b2} is CH₃, and Rₐ₃ and R_{b3} are

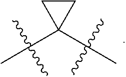 [SMILES: *C1(*)CC1]

In some variations of formula (3) described in the paragraphs above in which n₁ is 3, each of Rₐ₁ and Rₐ₂ is H and each of R_{b1} and R_{b2} is CH₃, and Rₐ₃ and R_{b3} are

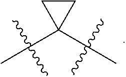 [SMILES: *C1(*)CC1]

In some variations of formula (3) described in the paragraphs above in which n₁ is 3, each of Rₐ₁, R_{b1}, Rₐ₂, and R_{b2}, is H and Rₐ₃ and R_{b3} are

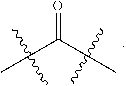 [SMILES: *C(*)=O]

In some variations of formula (3) described in the paragraphs above in which n₁ is 3, each of Rₐ₁, R_{b1}, Rₐ₂, and R_{b2}, is CH₃ and Rₐ₃ and R_{b3} are

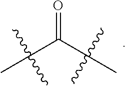 [SMILES: *C(*)=O]

In some variations of formula (3) described in the paragraphs above in which n₁ is 3, Rₐ₁ is H and each of Rₐ₂, R_{b1}, and R_{b2} is CH₃, and Rₐ₃ and R_{b3} are

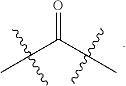 [SMILES: *C(*)=O]

In some variations of formula (3) described in the paragraphs above in which n₁ is 3, each of Rₐ₁ and Rₐ₂ is H and each of R_{b1} and R_{b2} is CH₃, and Rₐ₃ and R_{b3} are

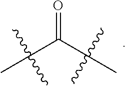 [SMILES: *C(*)=O]

In some variations of formula (3) described in the paragraphs above in which n₁ is 3, Rₐ₁ and R_{b1} are

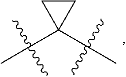 [SMILES: *C1(*)CC1]

Rₐ₂ and R_{b2} are

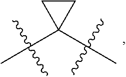 [SMILES: *C1(*)CC1]

and each of Rₐ₃ and R_{b3} is H. In some variations of formula (3) described in the paragraphs above in which n₁ is 3, Rₐ₁ and R_{b1} are

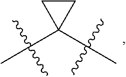 [SMILES: *C1(*)CC1]

Rₐ₂ and R_{b2} are

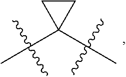 [SMILES: *C1(*)CC1]

and each of Rₐ₃ and R_{b3} is CH₃. In some variations of formula (3) described in the paragraphs above in which n₁ is 3, Rₐ₁ and R_{b1} are

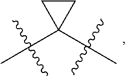 [SMILES: *C1(*)CC1]

Rₐ₂ and R_{b2} are

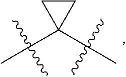 [SMILES: *C1(*)CC1]

Rₐ₃ is H, and R_{b3} is CH₃. In some variations of formula (3) described in the paragraphs above in which n₁ is 3, Rₐ₁ and R_{b1} are

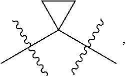 [SMILES: *C1(*)CC1]

Rₐ₂ and R_{b2} are

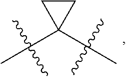 [SMILES: *C1(*)CC1]

and each of Rₐ₃ and R_{b3} is H. In some variations of formula (3) described in the paragraphs above in which n₁ is 3, Rₐ₁ and R_{b1} are

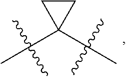 [SMILES: *C1(*)CC1]

Rₐ₂ and R_{b2} are

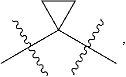 [SMILES: *C1(*)CC1]

and each of Rₐ₃ and R_{b3} is CH₃. In some variations of formula (3) described in the paragraphs above in which n₁ is 3, Rₐ₁ and R_{b1} are

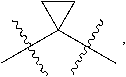 [SMILES: *C1(*)CC1]

Rₐ₂ and R_{b2} are

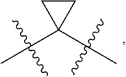 [SMILES: *C1(*)CC1]

Rₐ₃ is H, and R_{b3} is CH₃.

In some variations of formula (3) described in the paragraphs above in which n₁ is 3, Rₐ₁ and R_{b1} are

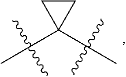 [SMILES: *C1(*)CC1]

Rₐ₂ and R_{b2} are

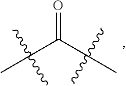 [SMILES: *C(*)=O]

and each of Rₐ₃ and R_{b3} is H. In some variations of formula (3) described in the paragraphs above in which n₁ is 3, Rₐ₁ and R_{b1} are

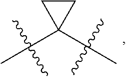 [SMILES: *C1(*)CC1]

Rₐ₂ and R_{b2} are

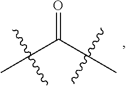 [SMILES: *C(*)=O]

and each of Rₐ₃ and R_{b3} is CH₃. In some variations of formula (3) described in the paragraphs above in which n₁ is 3, Rₐ₁ and R_{b1} are

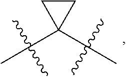 [SMILES: *C1(*)CC1]

Rₐ₂ and R_{b2} are

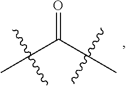 [SMILES: *C(*)=O]

Rₐ₃ is H, and R_{b3} is CH₃. In some variations of formula (3) described in the paragraphs above in which n₁ is 3, Rₐ₁ and R_{b1} are

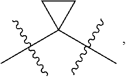 [SMILES: *C1(*)CC1]

Rₐ₂ and R_{b2} are

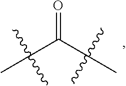 [SMILES: *C(*)=O]

and each of Rₐ₃ and R_{b3} is H. In some variations of formula (3) described in the paragraphs above in which n₁ is 3, Rₐ₁ and R_{b1} are

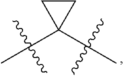 [SMILES: *C1(*)CC1]

Rₐ₂ and R_{b2} are

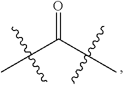 [SMILES: *C(*)=O]

and each of Rₐ₃ and R_{b3} is CH₃. In some variations of formula (3) described in the paragraphs above in which n₁ is 3, Rₐ₁ and R_{b1} are

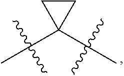 [SMILES: *C1(*)CC1]

Rₐ₂ and R_{b2} are

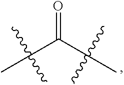 [SMILES: *C(*)=O]

Rₐ₃ is H, and R_{b3} is CH₃.

In some variations of formula (3) described in the paragraphs above in which n₁ is 3, Rₐ₁ and R_{b1} are

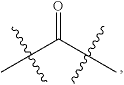 [SMILES: *C(*)=O]

Rₐ₂ and R_{b2} are

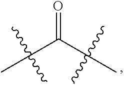 [SMILES: *C(*)=O]

and each of Rₐ₃ and R_{b3} is H. In some variations of formula (3) described in the paragraphs above in which n₁ is 3, Rₐ₁ and R_{b1} are

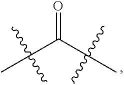 [SMILES: *C(*)=O]

Rₐ₂ and R_{b2} are

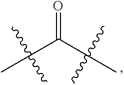 [SMILES: *C(*)=O]

and each of Rₐ₃ and R_{b3} is CH₃. In some variations of formula (3) described in the paragraphs above in which n₁ is 3, Rₐ₁ and R_{b1} are

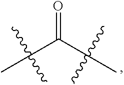 [SMILES: *C(*)=O]

Rₐ₂ and R_{b2} are

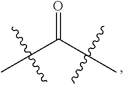 [SMILES: *C(*)=O]

Rₐ₃ is H, and R_{b3} is CH₃. In some variations of formula (3) described in the paragraphs above in which n₁ is 3, Rₐ₁ and R_{b1} are

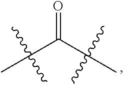 [SMILES: *C(*)=O]

Rₐ₂ and R_{b2} are

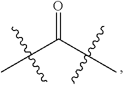 [SMILES: *C(*)=O]

and each of Rₐ₃ and R_{b3} is H. In some variations of formula (3) described in the paragraphs above in which n₁ is 3, Rₐ₁ and R_{b1} are

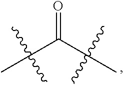 [SMILES: *C(*)=O]

Rₐ₂ and R_{b2} are

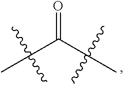 [SMILES: *C(*)=O]

and each of Rₐ₃ and R_{b3} is CH₃. In some variations of formula (3) described in the paragraphs above in which n₁ is 3, Rₐ₁ and R_{b1} are

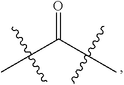 [SMILES: *C(*)=O]

Rₐ₂ and R_{b2} are

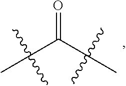 [SMILES: *C(*)=O]

Rₐ₃ is H, and R_{b3} is CH₃.

In some variations of formula (3) described in the paragraphs above in which n₁ is 3, Rₐ₁ and R_{b1} and Rₐ₂ and R_{b2} together are

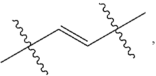 [SMILES: */C=C/*]

and each of Rₐ₃ and R_{b3} is H. In some variations of formula (3) described in the paragraphs above in which n₁ is 3, Rₐ₁ and R_{b1} and Rₐ₂ and R_{b2} together are

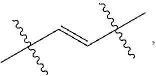 [SMILES: */C=C/*]

and each of Rₐ₃ and R_{b3} is CH₃. In some variations of formula (3) described in the paragraphs above in which n₁ is 3, Rₐ₁ and R_{b1} and Rₐ₂ and R_{b2} together are

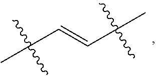 [SMILES: */C=C/*]

Rₐ₃ is H, and R_{b3} is CH₃. In some variations of formula (3) described in the paragraphs above in which n₁ is 3, Rₐ₁ and R_{b1} and Rₐ₂ and R_{b2} together are

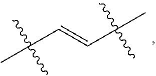 [SMILES: */C=C/*]

and Rₐ₃ and R_{b3} are

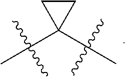 [SMILES: *C1(*)CC1]

In some variations of formula (3) described in the paragraphs above in which n₁ is 3, Rₐ₁ and R_{b1} and Rₐ₂ and R_{b2} together are

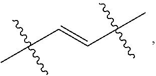 [SMILES: */C=C/*]

and Rₐ₃ and R_{b3} are

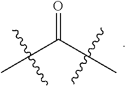 [SMILES: *C(*)=O]

In variations of formula (3) described in the paragraphs above in which n₁ is 4, the three instances of Rₐ and R_{b} are indicated as Rₐ₁ and R_{b1}; Rₐ₂ and R_{b2}; Rₐ₃ and R_{b3}; and Rₐ₄ and R_{b4}, respectively. In some variations of formula (3) described in the paragraphs above in which n₁ is 4, In some variations of formula (3) described in the paragraphs above in which n₁ is 4, each of Rₐ₁, R_{b1}, Rₐ₂, R_{b2}, Rₐ₃, R_{b3}, Rₐ₄, and R_{b4} is H. In some variations of formula (3) described in the paragraphs above in which n₁ is 4, each of Rₐ₁, R_{b1}, Rₐ₂, R_{b2}, Rₐ₃, R_{b3}, Rₐ₄, and R_{b4} is CH₃. In some variations of formula (3) described in the paragraphs above in which n₁ is 4, each of Rₐ₁, R_{b1}, Rₐ₂, R_{b2}, Rₐ₃, and R_{b3} is H and each of Rₐ₄ and R_{b4} is CH₃. In some variations of formula (3) described in the paragraphs above in which n₁ is 4, each of Rₐ₁, R_{b1}, Rₐ₂, and R_{b2} is H and each of Rₐ₃, R_{b3}, Rₐ₄, and R_{b4} is CH₃. In some variations of formula (3) described in the paragraphs above in which n₁ is 4, each of Rₐ₁, R_{b1}, Rₐ₂, R_{b2}, Rₐ₃, and R_{b3} is CH₃ and each of Rₐ₄ and R_{b4} is H.

In some variations of formula (3) described in the paragraphs above in which n₁ is 4, each of Rₐ₁, Rₐ₂, and Rₐ₃ is H and each of R_{b1}, R_{b2}, and R_{b3} is CH₃. In some variations of formula (3) described in the paragraphs above in which n₁ is 4, Rₐ₁ is H, R_{b1} is CH₃, and each of Rₐ₂, R_{b2}, Rₐ₃, and R_{b3} is CH₃. In some variations of formula (3) described in the paragraphs above in which n₁ is 4, Rₐ₁ is H, R_{b1} is CH₃, and each of Rₐ₂, R_{b2}, Rₐ₃, and R_{b3} is H. In some variations of formula (3) described in the paragraphs above in which n₁ is 4, each of Rₐ₁ and Rₐ₂ is H, each of R_{b1} and R_{b2} is CH₃, and each of Rₐ₃ and R_{b3} is H. In some variations of formula (3) described in the paragraphs above in which n₁ is 4, each of Rₐ₁ and Rₐ₂ is H, each of R_{b1} and R_{b2} is CH₃, and each of Rₐ₃ and R_{b3} is CH₃.

In some variations of formula (3) described in the paragraphs above in which n₁ is 4, Rₐ₁ and R_{b1} are H, Rₐ₂ and R_{b2} are H, Rₐ₃ and R_{b3} are H, and Rₐ₄ and R_{b4} are

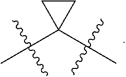 [SMILES: *C1(*)CC1]

In some variations of formula (3) described in the paragraphs above in which nⱼ is 4, Rₐ₁ and R_{b1} are H, Rₐ₂ and R_{b2} are H, Rₐ₃ and R_{b3} are CH₃, and Rₐ₄ and R_{b4} are

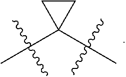 [SMILES: *C1(*)CC1]

In some variations of formula (3) described in the paragraphs above in which n₁ is 4, Rₐ₁ and R_{b1} are H, Rₐ₂ and R_{b2} are CH₃, Rₐ₃ and R_{b3} are CH₃, and Rₐ₄ and R_{b4} are

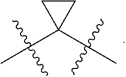 [SMILES: *C1(*)CC1]

In some variations of formula (3) described in the paragraphs above in which nⱼ is 4, Rₐ₁ and R_{b1} are CH₃, Rₐ₂ and R_{b2} are CH₃, Rₐ₃ and R_{b3} are CH₃, and Rₐ₄ and R_{b4} are

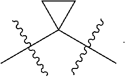 [SMILES: *C1(*)CC1]

In some variations of formula (3) described in the paragraphs above in which n₁ is 4, Rₐ₁ and R_{b1} are H, Rₐ₂ and R_{b2} are H, Rₐ₃ and R_{b3} are

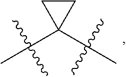 [SMILES: *C1(*)CC1]

and Rₐ₄ and R_{b4} are

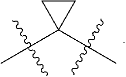 [SMILES: *C1(*)CC1]

In some variations of formula (3) described in the paragraphs above in which n₁ is 4, Rₐ₁ and R_{b1} are H, Rₐ₂ and R_{b2} are CH₃, Rₐ₃ and R_{b3} are

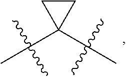 [SMILES: *C1(*)CC1]

and Rₐ₄ and R_{b4} are

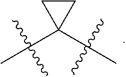 [SMILES: *C1(*)CC1]

In some variations of formula (3) described in the paragraphs above in which n₁ is 4, Rₐ₁ and R_{b1} are CH₃, Rₐ₂ and R_{b2} are CH₃, Rₐ₃ and R_{b3} are

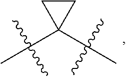 [SMILES: *C1(*)CC1]

and Rₐ₄ and R_{b4} are

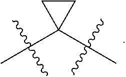 [SMILES: *C1(*)CC1]

In some variations of formula (3) described in the paragraphs above in which n₁ is 4, Rₐ₁ is H, R_{b1} is CH₃, Rₐ₂ and R_{b2} are CH₃, Rₐ₃ and R_{b3} are

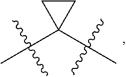 [SMILES: *C1(*)CC1]

and Rₐ₄ and R_{b4} are

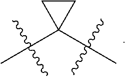 [SMILES: *C1(*)CC1]

In some variations of formula (3) described in the paragraphs above in which n₁ is 4, Rₐ₁ is H, R_{b1} is CH₃, Rₐ₂ is H, R_{b2} is CH₃, Rₐ₃ and R_{b3} are

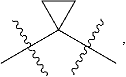 [SMILES: *C1(*)CC1]

and Rₐ₄ and R_{b4} are

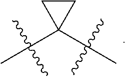 [SMILES: *C1(*)CC1]

In some variations of formula (3) described in the paragraphs above in which n₁ is 4, Rₐ₁ and R_{b1} are H, Rₐ₂ and R_{b2} are

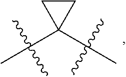 [SMILES: *C1(*)CC1]

Rₐ₃ and R_{b3} are

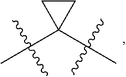 [SMILES: *C1(*)CC1]

and Rₐ₄ and R_{b4} are

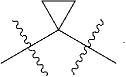 [SMILES: *C1(*)CC1]

In some variations of formula (3) described in the paragraphs above in which n₁ is 4, Rₐ₁ and R_{b1} are CH₃, Rₐ₂ and R_{b2} are

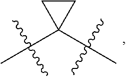 [SMILES: *C1(*)CC1]

Rₐ₃ and R_{b3} are

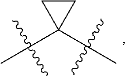 [SMILES: *C1(*)CC1]

and Rₐ₄ and R_{b4} are

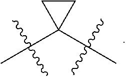 [SMILES: *C1(*)CC1]

In some variations of formula (3) described in the paragraphs above in which n₁ is 4, Rₐ₁ is H, R_{b1} is CH₃, Rₐ₂ and R_{b2} are

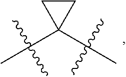 [SMILES: *C1(*)CC1]

Rₐ₃ and R_{b3} are

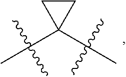 [SMILES: *C1(*)CC1]

and Rₐ₄ and R_{b4} are

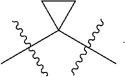 [SMILES: *C1(*)CC1]

In some variations of formula (3) described in the paragraphs above in which n₁ is 4, Rₐ₁ and R_{b1} are

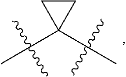 [SMILES: *C1(*)CC1]

Rₐ₂ and R_{b2} are

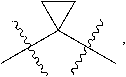 [SMILES: *C1(*)CC1]

Rₐ₃ and R_{b3} are

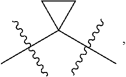 [SMILES: *C1(*)CC1]

and Rₐ₄ and R_{b4} are

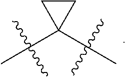 [SMILES: *C1(*)CC1]

In some variations of formula (3) described in the paragraphs above in which n₁ is 4, Rₐ₁ and R_{b1} are H, Rₐ₂ and R_{b2} are H, Rₐ₃ and R_{b3} are H, and Rₐ₄ and R_{b4} are

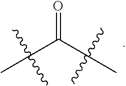 [SMILES: *C(*)=O]

In some variations of formula (3) described in the paragraphs above in which n₁ is 4, Rₐ₁ and R_{b1} are H, Rₐ₂ and R_{b2} are H, Rₐ₃ and R_{b3} are CH₃, and Rₐ₄ and R_{b4} are

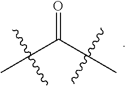 [SMILES: *C(*)=O]

In some variations of formula (3) described in the paragraphs above in which n₁ is 4, Rₐ₁ and R_{b1} are H, Rₐ₂ and R_{b2} are CH₃, Rₐ₃ and R_{b3} are CH₃, and Rₐ₄ and R_{b4} are

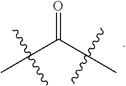 [SMILES: *C(*)=O]

In some variations of formula (3) described in the paragraphs above in which n₁ is 4, Rₐ₁ and R_{b1} are CH₃, Rₐ₂ and R_{b2} are CH₃, Rₐ₃ and R_{b3} are CH₃, and Rₐ₄ and R_{b4} are

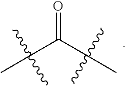 [SMILES: *C(*)=O]

In some variations of formula (3) described in the paragraphs above in which n₁ is 4, Rₐ₁ and R_{b1} are H, Rₐ₂ and R_{b2} are H, Rₐ₃ and R_{b3} are

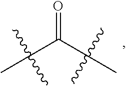 [SMILES: *C(*)=O]

and Rₐ₄ and R_{b4} are v. In some variations of formula (3) described in the paragraphs above in which n₁ is 4, Rₐ₁ and R_{b1} are H, Rₐ₂ and R_{b2} are CH₃, Rₐ₃ and R_{b3} are

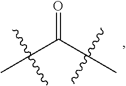 [SMILES: *C(*)=O]

and Rₐ₄ and R_{b4} are

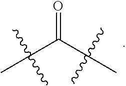 [SMILES: *C(*)=O]

In some variations of formula (3) described in the paragraphs above in which n₁ is 4, Rₐ₁ and R_{b1} are CH₃, Rₐ₂ and R_{b2} are CH₃, Rₐ₃ and R_{b3} are

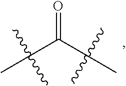 [SMILES: *C(*)=O]

and Rₐ₄ and R_{b4} are

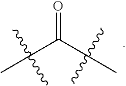 [SMILES: *C(*)=O]

In some variations of formula (3) described in the paragraphs above in which n₁ is 4, Rₐ₁ is H, R_{b1} is CH₃, Rₐ₂ and R_{b2} are CH₃, Rₐ₃ and R_{b3} are

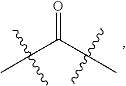 [SMILES: *C(*)=O]

and Rₐ₄ and R_{b4} are

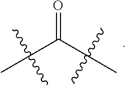 [SMILES: *C(*)=O]

In some variations of formula (3) described in the paragraphs above in which n₁ is 4, Rₐ₁ is H, R_{b1} is CH₃, Rₐ₂ is H, R_{b2} is CH₃, Rₐ₃ and R_{b3} are

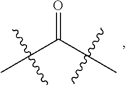 [SMILES: *C(*)=O]

and Rₐ₄ and R_{b4} are

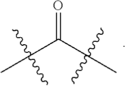 [SMILES: *C(*)=O]

In some variations of formula (3) described in the paragraphs above in which n₁ is 4, Rₐ₁ and R_{b1} are H, Rₐ₂ and R_{b2} are

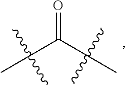 [SMILES: *C(*)=O]

Rₐ₃ and R_{b3} are

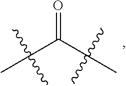 [SMILES: *C(*)=O]

and Rₐ₄ and R_{b4} are

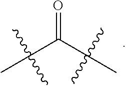 [SMILES: *C(*)=O]

In some variations of formula (3) described in the paragraphs above in which n₁ is 4, Rₐ₁ and R_{b1} are CH₃, Rₐ₂ and R_{b2} are

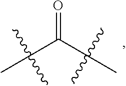 [SMILES: *C(*)=O]

Rₐ₃ and R_{b3} are

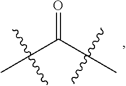 [SMILES: *C(*)=O]

and Rₐ₄ and R_{b4} are

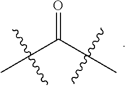 [SMILES: *C(*)=O]

In some variations of formula (3) described in the paragraphs above in which n₁ is 4, Rₐ₁ is H, R_{b1} is CH₃, Rₐ₂ and R_{b2} are

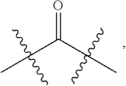 [SMILES: *C(*)=O]

Rₐ₃ and R_{b3} are

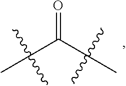 [SMILES: *C(*)=O]

and Rₐ₄ and R_{b4} are

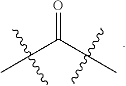 [SMILES: *C(*)=O]

In some variations of formula (3) described in the paragraphs above in which n₁ is 4, Rₐ₁ and R_{b1} are

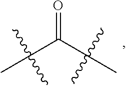 [SMILES: *C(*)=O]

Rₐ₂ and R_{b2} are

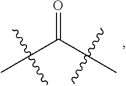 [SMILES: *C(*)=O]

Rₐ₃ and R_{b3} are

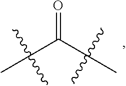 [SMILES: *C(*)=O]

and Rₐ₄ and R_{b4} are

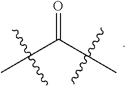 [SMILES: *C(*)=O]

In some variations of formula (3) described in the paragraphs above in which n₁ is 4, Rₐ₁ and R_{b1} are

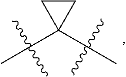 [SMILES: *C1(*)CC1]

Rₐ₂ and R_{b2} are H, Rₐ₃ and R_{b3} are H, and Rₐ₄ and R_{b4} are

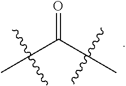 [SMILES: *C(*)=O]

In some variations of formula (3) described in the paragraphs above in which n₁ is 4, Rₐ₁ and R_{b1} are

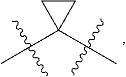 [SMILES: *C1(*)CC1]

Rₐ₂ and R_{b2} are CH₃, Rₐ₃ and R_{b3} are CH₃, and Rₐ₄ and R_{b4} are

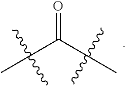 [SMILES: *C(*)=O]

In some variations of formula (3) described in the paragraphs above in which n₁ is 4, Rₐ₁ and R_{b1} are

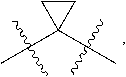 [SMILES: *C1(*)CC1]

Rₐ₂ and R_{b2} are H, Rₐ₃ and R_{b3} are CH₃, and Rₐ₄ and R_{b4} are

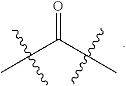 [SMILES: *C(*)=O]

In some variations of formula (3) described in the paragraphs above in which n₁ is 4, Rₐ₁ and R_{b1} are

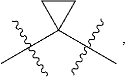 [SMILES: *C1(*)CC1]

Rₐ₂ and R_{b2} are

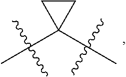 [SMILES: *C1(*)CC1]

Rₐ₃ and R_{b3} are H, and Rₐ₄ and R_{b4} are

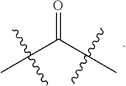 [SMILES: *C(*)=O]

In some variations of formula (3) described in the paragraphs above in which n₁ is 4, Rₐ₁ and R_{b1} are

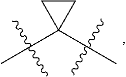 [SMILES: *C1(*)CC1]

Rₐ₂ and R_{b2} are

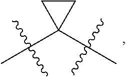 [SMILES: *C1(*)CC1]

Rₐ₃ and R_{b3} are CH₃, and Rₐ₄ and R_{b4} are

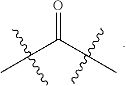 [SMILES: *C(*)=O]

In some variations of formula (3) described in the paragraphs above in which n₁ is 4, Rₐ₁ and R_{b1} are

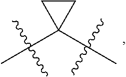 [SMILES: *C1(*)CC1]

Rₐ₂ and R_{b2} are

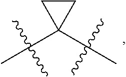 [SMILES: *C1(*)CC1]

Rₐ₃ and R_{b3} are CH₃, and Rₐ₄ and R_{b4} are

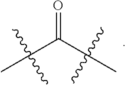 [SMILES: *C(*)=O]

In some variations of formula (3) described in the paragraphs above in which n₁ is 4, Rₐ₁ and R_{b1} are

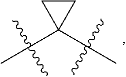 [SMILES: *C1(*)CC1]

Rₐ₂ and R_{b2} are

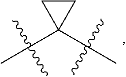 [SMILES: *C1(*)CC1]

Rₐ₃ and R_{b3} are H, and Rₐ₄ and R_{b4} are

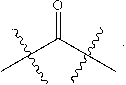 [SMILES: *C(*)=O]

In some variations of formula (3) described in the paragraphs above in which n₁ is 4, Rₐ₁ and R_{b1} are

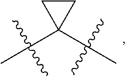 [SMILES: *C1(*)CC1]

Rₐ₂ and R_{b2} are

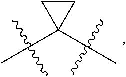 [SMILES: *C1(*)CC1]

Rₐ₃ and R_{b3} are

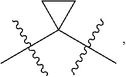 [SMILES: *C1(*)CC1]

and Rₐ₄ and R_{b4} are

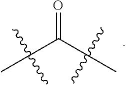 [SMILES: *C(*)=O]

In some variations of formula (3) described in the paragraphs above in which n₁ is 4, Rₐ₁ and R_{b1} are

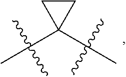 [SMILES: *C1(*)CC1]

Rₐ₂ and R_{b2} are

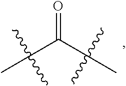 [SMILES: *C(*)=O]

Rₐ₃ and R_{b3} are H, and Rₐ₄ and R_{b4} are

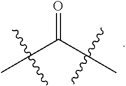 [SMILES: *C(*)=O]

In some variations of formula (3) described in the paragraphs above in which n₁ is 4, Rₐ₁ and R_{b1} are

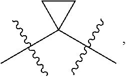 [SMILES: *C1(*)CC1]

Rₐ₂ and R_{b2} are

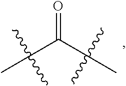 [SMILES: *C(*)=O]

Rₐ₃ and R_{b3} are CH₃, and Rₐ₄ and R_{b4} are

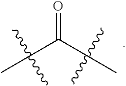 [SMILES: *C(*)=O]

In some variations of formula (3) described in the paragraphs above in which n₁ is 4, Rₐ₁ and R_{b1} are

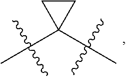 [SMILES: *C1(*)CC1]

Rₐ₂ and R_{b2} are

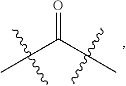 [SMILES: *C(*)=O]

Rₐ₃ and R_{b3} are CH₃, and Rₐ₄ and R_{b4} are

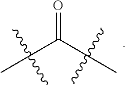 [SMILES: *C(*)=O]

In some variations of formula (3) described in the paragraphs above in which n₁ is 4, Rₐ₁ and R_{b1} are

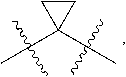 [SMILES: *C1(*)CC1]

Rₐ₂ and R_{b2} are

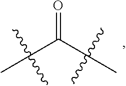 [SMILES: *C(*)=O]

Rₐ₃ and R_{b3} are H, and Rₐ₄ and R_{b4} are

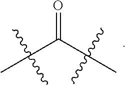 [SMILES: *C(*)=O]

In some variations of formula (3) described in the paragraphs above in which n₁ is 4, Rₐ₁ and R_{b1} are

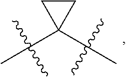 [SMILES: *C1(*)CC1]

Rₐ₂ and R_{b2} are

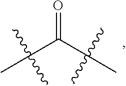 [SMILES: *C(*)=O]

Rₐ₃ and R_{b3} are

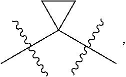 [SMILES: *C1(*)CC1]

and Rₐ₄ and R_{b4} are

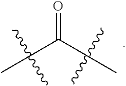 [SMILES: *C(*)=O]

In some variations of formula (3) described in the paragraphs above in which n₁ is 4, Rₐ₁ and R_{b1} are

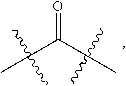 [SMILES: *C(*)=O]

Rₐ₂ and R_{b2} are

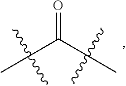 [SMILES: *C(*)=O]

Rₐ₃ and R_{b3} are H, and Rₐ₄ and R_{b4} are

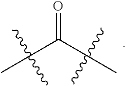 [SMILES: *C(*)=O]

In some variations of formula (3) described in the paragraphs above in which n₁ is 4, Rₐ₁ and R_{b1} are

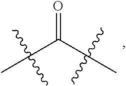 [SMILES: *C(*)=O]

Rₐ₂ and R_{b2} are

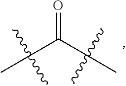 [SMILES: *C(*)=O]

Rₐ₃ and R_{b3} are CH₃, and Rₐ₄ and R_{b4} are

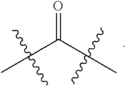 [SMILES: *C(*)=O]

In some variations of formula (3) described in the paragraphs above in which n₁ is 4, Rₐ₁ and R_{b1} are

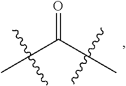 [SMILES: *C(*)=O]

Rₐ₂ and R_{b2} are

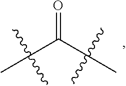 [SMILES: *C(*)=O]

Rₐ₃ and R_{b3} are CH₃, and Rₐ₄ and R_{b4} are

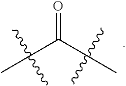 [SMILES: *C(*)=O]

In some variations of formula (3) described in the paragraphs above in which n₁ is 4, Rₐ₁ and R_{b1} are

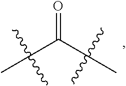 [SMILES: *C(*)=O]

Rₐ₂ and R_{b2} are

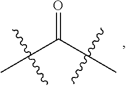 [SMILES: *C(*)=O]

Rₐ₃ and R_{b3} are H, and Rₐ₄ and R_{b4} are

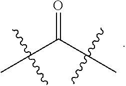 [SMILES: *C(*)=O]

In some variations of formula (3) described in the paragraphs above in which n₁ is 4, Rₐ₁ and R_{b1} are

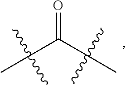 [SMILES: *C(*)=O]

Rₐ₂ and R_{b2} are

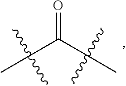 [SMILES: *C(*)=O]

Rₐ₃ and R_{b3} are

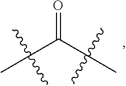 [SMILES: *C(*)=O]

and Rₐ₄ and R_{b4} are

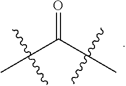 [SMILES: *C(*)=O]

In some variations of formula (3) described in the paragraphs above in which n₁ is 4, Rₐ₁ and R_{b1} are H, Rₐ₂ and R_{b2} are H, and Rₐ₃ and R_{b3} and Rₐ₄ and R_{b4} together are

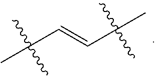 [SMILES: */C=C/*]

In some variations of formula (3) described in the paragraphs above in which n₁ is 4, Rₐ₁ and R_{b1} are H, Rₐ₂ and R_{b2} are CH₃, and Rₐ₃ and R_{b3} and Rₐ₄ and R_{b4} together are

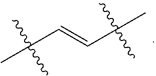 [SMILES: */C=C/*]

In some variations of formula (3) described in the paragraphs above in which n₁ is 4, Rₐ₁ and Rₐ₂ are H, R_{b1} and R_{b2} are CH₃, and Rₐ₃ and R_{b3} and Rₐ₄ and R_{b4} together are

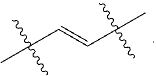 [SMILES: */C=C/*]

In some variations of formula (3) described in the paragraphs above in which n₁ is 4, Rₐ₁ and R_{b1} are H, Rₐ₂ and R_{b2} are

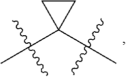 [SMILES: *C1(*)CC1]

and Rₐ₃ and R_{b3} and Rₐ₄ and R_{b4} together are

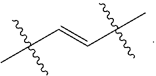 [SMILES: */C=C/*]

In some variations of formula (3) described in the paragraphs above in which n₁ is 4, Rₐ₁ and R_{b1} are

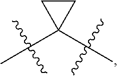

Rₐ₂ and R_{b2} are CH₃, and Rₐ₃ and R_{b3} and Rₐ₄ and R_{b4} together are

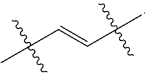

In some variations of formula (3) described in the paragraphs above in which n₁ is 4, Rₐ₁ is H, R_{b1} is CH₃, Rₐ₂ and R_{b2} are

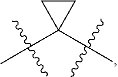

and Rₐ₃ and R_{b3} and Rₐ₄ and R_{b4} together are

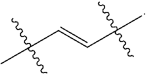

In some variations of formula (3) described in the paragraphs above in which n₁ is 4, Rₐ₁ and R_{b1} are H, Rₐ₂ and R_{b2} are

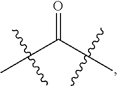

and Rₐ₃ and R_{b3} and Rₐ₄ and R_{b4} together are

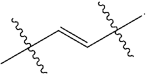

In some variations of formula (3) described in the paragraphs above in which n₁ is 4, Rₐ₁ and R_{b1} are

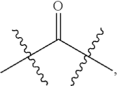

Rₐ₂ and R_{b2} are CH₃, and Rₐ₃ and R_{b3} and Rₐ₄ and R_{b4} together are

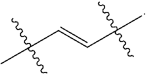

In some variations of formula (3) described in the paragraphs above in which n₁ is 4, Rₐ₁ is H, R_{b1} is CH₃, Rₐ₂ and R_{b2} are

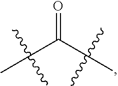

and Rₐ₃ and R_{b3} and Rₐ₄ and R_{b4} together are

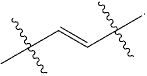

In some variations of formula (3) described in the paragraphs above in which n₁ is 4, Rₐ₁ and R_{b1} are

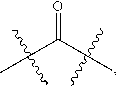

Rₐ₂ and R_{b2} are

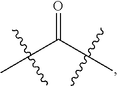

and Rₐ₃ and R_{b3} and Rₐ₄ and R_{b4} together are

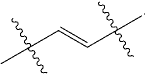

In some variations of formula (3) described in the paragraphs above in which n₁ is 4, Rₐ₁ and R_{b1} are

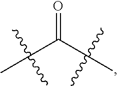

Rₐ₂ and R_{b2} are

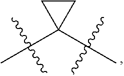 [SMILES: *C1(*)CC1]

and Rₐ₃ and R_{b3} and Rₐ₄ and R_{b4} together are

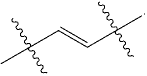

In some variations of formula (3) described in the paragraphs above in which n₁ is 4, Rₐ₁ and R_{b1} are

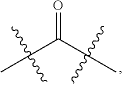 [SMILES: *C(*)=O]

Rₐ₂ and R_{b2} are

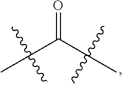 [SMILES: *C(*)=O]

and Rₐ₃ and R_{b3} and Rₐ₄ and R_{b4} together are

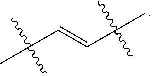 [SMILES: */C=C/*]

In some variations of formula (3) described in the paragraphs above in which n₁ is 4, Rₐ₁ and R_{b1} are

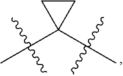 [SMILES: *C1(*)CC1]

Rₐ₂ and R_{b2} are

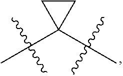 [SMILES: *C1(*)CC1]

and Rₐ₃ and R_{b3} and Rₐ₄ and R_{b4} together are

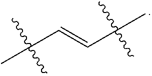 [SMILES: */C=C/*]

In some variations of formula (3) described in the paragraphs above in which n₁ is 4, Rₐ₁ and R_{b1} and Rₐ₂ and R_{b2} together are

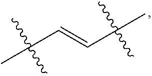 [SMILES: */C=C/*]

and Rₐ₃ and R_{b3} and Rₐ₄ and R_{b4} together are

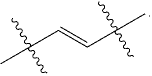 [SMILES: */C=C/*]

In some variations of formula (3) described in the paragraphs above, D1 is safranin-O. In some variations of formula (3) described in the paragraphs above, D2 is safranin-O. In some variations of formula (3) described in the paragraphs above, D1 and D2 are safranin-O.

In some variations of formula (3) described in the paragraph above, the pendant phenyl ring of D1 is unsubstituted. In some variations of formula (3) described in the paragraph above, the pendant phenyl ring of D1 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D1 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D1 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (3) described in the paragraph above, the pendant phenyl ring of D2 is unsubstituted. In some variations of formula (3) described in the paragraph above, the pendant phenyl ring of D2 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D2 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D2 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

Some cationic dye dimers fall within formula (4):

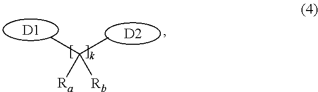

in which each of D1 and D2 is a cationic dye moiety; k is 2-10 and, for each independent instance of Rₐ and R_{b}, Rₐ and R_{b} (1) independently are H or CH₃, or (2) Rₐ and R_{b} are

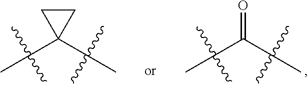

or (3) two of CRₐR_{b} are

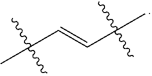 [SMILES: */C=C/*]

In some variations of formula (4), D1 and D2 are different cationic dye moieties. In other variations of formula (4), D1 and D2 are the same cationic dye moiety. In some variations of formula (4), D1 and D2 are independently selected from the group consisting of safranin-O, toluidine blue, azure A, azure B, azure C, acridine orange, acriflavine, and methylene blue.

In some variations of formula (4) described in the paragraphs above, k is 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, 9-10, 2, 3, 4, 5, 6, 7, 8, 9, or 10.

In some variations of formula (4) described in the paragraphs above, each Rₐ is H and each R_{b} is H. In some variations of formula (4) described in the paragraphs above, each Rₐ is H and each R_{b} is CH₃. In some variations of formula (4) described in the paragraphs above, each Rₐ and R_{b} is

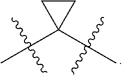 [SMILES: *C1(*)CC1]

In some variations of formula (4) described in the paragraphs above, each Rₐ and R_{b} is

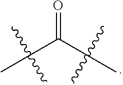 [SMILES: *C(*)=O]

In some variations of formula (4) described in the paragraphs above, each two of CRₐR_{b} are

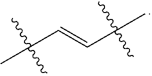 [SMILES: */C=C/*]

In some variations of formula (4) described in the paragraphs above, in a first occurrence of Rₐ and R_{b}, each of Rₐ and R_{b} is H, and the remaining occurrences of Rₐ and R_{b} are as defined above for formula (4). In some variations of formula (4) described in the paragraphs above, in a first occurrence of Rₐ and R_{b}, each of Rₐ and R_{b} is CH₃, and the remaining occurrences of Rₐ and R_{b} are as defined above for formula (4). In some variations of formula (4) described in the paragraphs above, in a first occurrence of Rₐ and R_{b}, a first Rₐ is H, a first R_{b} is CH₃, and the remaining occurrences of Rₐ and R_{b} are as defined above for formula (4). In some variations of formula (4) described in the paragraphs above, in a first occurrence of Rₐ and R_{b}, Rₐ and R_{b} are

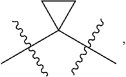 [SMILES: *C1(*)CC1]

and the remaining occurrences of Rₐ and R_{b} are as defined above for formula (4). In some variations of formula (4) described in the paragraphs above, in a first occurrence of Rₐ and R_{b}, Rₐ and R_{b} are

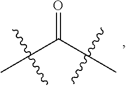 [SMILES: *C(*)=O]

and the remaining occurrences of Rₐ and R_{b} are as defined above for formula (4).

In some variations of formula (4) described in the paragraphs above, in two occurrences of Rₐ and R_{b}, each of Rₐ and R_{b} is H, and the remaining occurrences of Rₐ and R_{b} are as defined above for formula (4). In some variations of formula (4) described in the paragraphs above, in two occurrences of Rₐ and R_{b}, each of Rₐ and R_{b} is CH₃, and the remaining occurrences of Rₐ and R_{b} are as defined above for formula (4). In some variations of formula (4) described in the paragraphs above, in two occurrences of Rₐ and R_{b}, two of Rₐ are H, two of R_{b} are CH₃, and the remaining occurrences of Rₐ and R_{b} are as defined above for formula (4). In some variations of formula (4) described in the paragraphs above, in two occurrences of Rₐ and R_{b}, Rₐ and R_{b} are

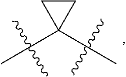 [SMILES: *C1(*)CC1]

and the remaining occurrences of Rₐ and R_{b} are as defined above for formula (4). In some variations of formula (4) described in the paragraphs above, in two occurrences of Rₐ and R_{b}, Rₐ and R_{b} are

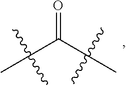 [SMILES: *C(*)=O]

and the remaining occurrences of Rₐ and R_{b} are as defined above for formula (4).

In some variations of formula (4) described in the paragraphs above, in three occurrences of Rₐ and R_{b}, each of Rₐ and R_{b} is H, and the remaining occurrences of Rₐ and R_{b} are as defined above for formula (4). In some variations of formula (4) described in the paragraphs above, in three occurrences of Rₐ and R_{b}, each of Rₐ and R_{b} is CH₃, and the remaining occurrences of Rₐ and R_{b} are as defined above for formula (4). In some variations of formula (4) described in the paragraphs above, in three occurrences of Rₐ and R_{b}, three of Rₐ are H, three of R_{b} are CH₃, and the remaining occurrences of Rₐ and R_{b} are as defined above for formula (4). In some variations of formula (4) described in the paragraphs above, in three occurrences of Rₐ and R_{b}, Rₐ and R_{b} are

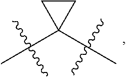 [SMILES: *C1(*)CC1]

and the remaining occurrences of Rₐ and R_{b} are as defined above for formula (4). In some variations of formula (4) described in the paragraphs above, in three occurrences of Rₐ and R_{b}, Rₐ and R_{b} are

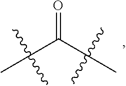 [SMILES: *C(*)=O]

and the remaining occurrences of Rₐ and R_{b} are as defined above for formula (4).

In some variations of formula (4) described in the paragraphs above, in four occurrences of Rₐ and R_{b}, each of Rₐ and R_{b} is H, and the remaining occurrences of Rₐ and R_{b} are as defined above for formula (4). In some variations of formula (4) described in the paragraphs above, in four occurrences of Rₐ and R_{b}, each of Rₐ and R_{b} is CH₃, and the remaining occurrences of Rₐ and R_{b} are as defined above for formula (4). In some variations of formula (4) described in the paragraphs above, in four occurrences of Rₐ and R_{b}, four of Rₐ are H, four of R_{b} are CH₃, and the remaining occurrences of Rₐ and R_{b} are as defined above for formula (4). In some variations of formula (4) described in the paragraphs above, in four occurrences of Rₐ and R_{b}, Rₐ and R_{b} are

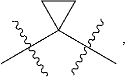 [SMILES: *C1(*)CC1]

and the remaining occurrences of Rₐ and R_{b} are as defined above for formula (4). In some variations of formula (4) described in the paragraphs above, in four occurrences of Rₐ and R_{b}, Rₐ and R_{b} are

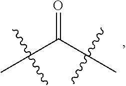 [SMILES: *C(*)=O]

and the remaining occurrences of Rₐ and R_{b} are as defined above for formula (4).

In some variations of formula (4) described in the paragraphs above, in five occurrences of Rₐ and R_{b}, each of Rₐ and R_{b} is H, and the remaining occurrences of Rₐ and R_{b} are as defined above for formula (4). In some variations of formula (4) described in the paragraphs above, in five occurrences of Rₐ and R_{b}, each of Rₐ and R_{b} is CH₃, and the remaining occurrences of Rₐ and R_{b} are as defined above for formula (4). In some variations of formula (4) described in the paragraphs above, in five occurrences of Rₐ and R_{b}, five of Rₐ are H, five of R_{b} are CH₃, and the remaining occurrences of Rₐ and R_{b} are as defined above for formula (4). In some variations of formula (4) described in the paragraphs above, in five occurrences of Rₐ and R_{b}, Rₐ and R_{b} are

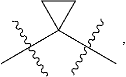 [SMILES: *C1(*)CC1]

and the remaining occurrences of Rₐ and R_{b} are as defined above for formula (4). In some variations of formula (4) described in the paragraphs above, in five occurrences of Rₐ and R_{b}, Rₐ and R_{b} are

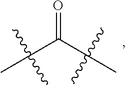 [SMILES: *C(*)=O]

and the remaining occurrences of Rₐ and R_{b} are as defined above for formula (4).

In some variations of formula (4) described in the paragraphs above, in six occurrences of Rₐ and R_{b}, each of Rₐ and R_{b} is H, and the remaining occurrences of Rₐ and R_{b} are as defined above for formula (4). In some variations of formula (4) described in the paragraphs above, in six occurrences of Rₐ and R_{b}, each of Rₐ and R_{b} is CH₃, and the remaining occurrences of Rₐ and R_{b} are as defined above for formula (4). In some variations of formula (4) described in the paragraphs above, in six occurrences of Rₐ and R_{b}, six of Rₐ are H, six of R_{b} are CH₃, and the remaining occurrences of Rₐ and R_{b} are as defined above for formula (4). In some variations of formula (4) described in the paragraphs above, in six occurrences of Rₐ and R_{b}, Rₐ and R_{b} are

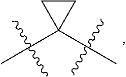 [SMILES: *C1(*)CC1]

and the remaining occurrences of Rₐ and R_{b} are as defined above for formula (4). In some variations of formula (4) described in the paragraphs above, in six occurrences of Rₐ and R_{b}, Rₐ and R_{b} are

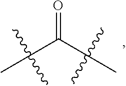 [SMILES: *C(*)=O]

and the remaining occurrences of Rₐ and R_{b} are as defined above for formula (4).

In some variations of formula (4) described in the paragraphs above, in seven occurrences of Rₐ and R_{b}, each of Rₐ and R_{b} is H, and the remaining occurrences of Rₐ and R_{b} are as defined above for formula (4). In some variations of formula (4) described in the paragraphs above, in seven occurrences of Rₐ and R_{b}, each of Rₐ and R_{b} is CH₃, and the remaining occurrences of Rₐ and R_{b} are as defined above for formula (4). In some variations of formula (4) described in the paragraphs above, in seven occurrences of Rₐ and R_{b}, seven of Rₐ are H, seven of R_{b} are CH₃, and the remaining occurrences of Rₐ and R_{b} are as defined above for formula (4). In some variations of formula (4) described in the paragraphs above, in seven occurrences of Rₐ and R_{b}, Rₐ and R_{b} are

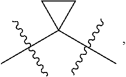 [SMILES: *C1(*)CC1]

and the remaining occurrences of Rₐ and R_{b} are as defined above for formula (4). In some variations of formula (4) described in the paragraphs above, in seven occurrences of Rₐ and R_{b}, Rₐ and R_{b} are

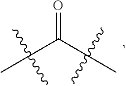 [SMILES: *C(*)=O]

and the remaining occurrences of Rₐ and R_{b} are as defined above for formula (4).

In some variations of formula (4) described in the paragraphs above, in eight occurrences of Rₐ and R_{b}, each of Rₐ and R_{b} is H, and the remaining occurrences of Rₐ and R_{b} are as defined above for formula (4). In some variations of formula (4) described in the paragraphs above, in eight occurrences of Rₐ and R_{b}, each of Rₐ and R_{b} is CH₃, and the remaining occurrences of Rₐ and R_{b} are as defined above for formula (4). In some variations of formula (4) described in the paragraphs above, in eight occurrences of Rₐ and R_{b}, eight of Rₐ are H, eight of R_{b} are CH₃, and the remaining occurrences of Rₐ and R_{b} are as defined above for formula (4). In some variations of formula (4) described in the paragraphs above, in eight occurrences of Rₐ and R_{b}, Rₐ and R_{b} are

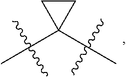 [SMILES: *C1(*)CC1]

and the remaining occurrences of Rₐ and R_{b} are as defined above for formula (4). In some variations of formula (4) described in the paragraphs above, in eight occurrences of Rₐand R_{b}, Rₐ and R_{b} are

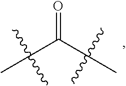 [SMILES: *C(*)=O]

and the remaining occurrences of Rₐ and R_{b} are as defined above for formula (4).

In some variations of formula (4) described in the paragraphs above, in nine occurrences of Rₐ and R_{b}, each of Rₐ and R_{b} is H, and the remaining occurrences of Rₐ and R_{b} are as defined above for formula (4). In some variations of formula (4) described in the paragraphs above, in nine occurrences of Rₐ and R_{b}, each of Rₐ and R_{b} is CH₃, and the remaining occurrences of Rₐ and R_{b} are as defined above for formula (4). In some variations of formula (4) described in the paragraphs above, in nine occurrences of Rₐ and R_{b}, nine of Rₐ are H, nine of R_{b} are CH₃, and the remaining occurrences of Rₐ and R_{b} are as defined above for formula (4). In some variations of formula (4) described in the paragraphs above, in nine occurrences of Rₐ and R_{b}, Rₐ and R_{b} are

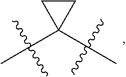 [SMILES: *C1(*)CC1]

and the remaining occurrences of Rₐ and R_{b} are as defined above for formula (4). In some variations of formula (4) described in the paragraphs above, in nine occurrences of Rₐ and R_{b}, Rₐ and R_{b} are

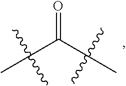 [SMILES: *C(*)=O]

and the remaining occurrences of Rₐ and R_{b} are as defined above for formula (4).

In some variations of formula (4) described in the paragraphs above, in ten occurrences of Rₐ and R_{b}, each of Rₐ and R_{b} is H, and the remaining occurrences of Rₐ and R_{b} are as defined above for formula (4). In some variations of formula (4) described in the paragraphs above, in ten occurrences of Rₐ and R_{b}, each of Rₐ and R_{b} is CH₃, and the remaining occurrences of Rₐ and R_{b} are as defined above for formula (4). In some variations of formula (4) described in the paragraphs above, in ten occurrences of Rₐ and R_{b}, ten of Rₐ are H, ten of R_{b} are CH₃, and the remaining occurrences of Rₐ and R_{b} are as defined above for formula (4). In some variations of formula (4) described in the paragraphs above, in ten occurrences of Rₐ and R_{b}, Rₐ and R_{b} are

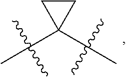 [SMILES: *C1(*)CC1]

and the remaining occurrences of Rₐ and R_{b} are as defined above for formula (4). In some variations of formula (4) described in the paragraphs above, in ten occurrences of Rₐ and R_{b}, Rₐ and R_{b} are

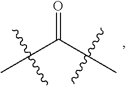 [SMILES: *C(*)=O]

and the remaining occurrences of Rₐ and R_{b} are as defined above for formula (4).

In some variations of formula (4) described in the paragraphs above, two occurrences of Rₐ and R_{b} are

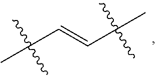 [SMILES: */C=C/*]

and the remaining occurrences of Rₐ and R_{b} are as defined above for formula (4). In some variations of formula (4) described in the paragraphs above, four occurrences of Rₐ and R_{b} are

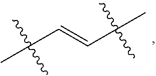 [SMILES: */C=C/*]

and the remaining occurrences of Rₐ and R_{b} are as defined above for formula (4). In some variations of formula (4) described in the paragraphs above, six occurrences of Rₐ and R_{b} are

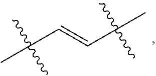 [SMILES: */C=C/*]

and the remaining occurrences of Rₐ and R_{b} are as defined above for formula (4). In some variations of formula (4) described in the paragraphs above, eight occurrences of Rₐ and R_{b} are

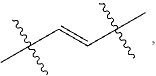 [SMILES: */C=C/*]

and the remaining occurrences of Rₐ and R_{b} are as defined above for formula (4). In some variations of formula (4) described in the paragraphs above, ten occurrences of Rₐ and R_{b} are

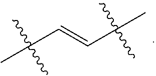 [SMILES: */C=C/*]

In some variations of formula (4) described in the paragraphs above, D1 is safranin-O. In some variations of formula (4) described in the paragraphs above, D2 is safranin-O. In some variations of formula (4) described in the paragraphs above, D1 and D2 are safranin-O.

In some variations of formula (4) described in the paragraph above, the pendant phenyl ring of D1 is unsubstituted. In some variations of formula (4) described in the paragraph above, the pendant phenyl ring of D1 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D1 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D1 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (4) described in the paragraph above, the pendant phenyl ring of D2 is unsubstituted. In some variations of formula (4) described in the paragraph above, the pendant phenyl ring of D2 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D2 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D2 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some of the variations of formula (4) described above, D1 and D2 are safranin-O moieties, as shown in formula (4a):

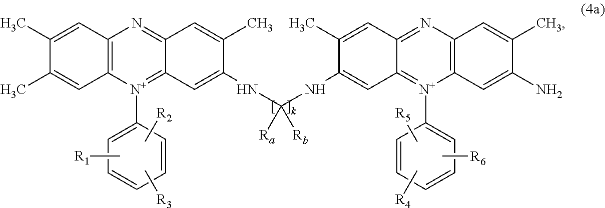

in which k, Rₐ, and R_{b} are as described in the paragraphs above, R₁, R₂, R₃, R₄, R₅, and R₆ independently are absent or independently are selected from —NH₂, —NHR, —NR₂, —OH, —O⁻, —NHCOCH₃, —NHCOR, —OCH₃, —OR, —C₂H₅, —R, —C₆H₅, —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR), and R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some of the variations of formula (4a), k is 2-10, each Rₐ and R_{b} is H, and R₁ to R₆ are as described above for formula (4). In some of these variations, k is 6 or 8, each Rₐ and R_{b} is H, and each of R₁ to R₆ independently is absent or is a halo. In particular variations, k is 6 or 8, each Rₐ and R_{b} is H, and R₁ to R₆ are all absent.

In some of the variations of formula (4a), k is 2-10, Rₐ and R_{b} are either H or

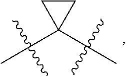 [SMILES: *C1(*)CC1]

and R₁ to R₆ are as described above for formula (4). In some of these variations, k is 6 or 8, Rₐ and R_{b} are either H or

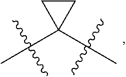 [SMILES: *C1(*)CC1]

and each of R₁ to R₆ independently is absent or is a halo. In particular variations, k is 6 or 8, Rₐ and R_{b} are either H or

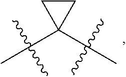 [SMILES: *C1(*)CC1]

and R₁ to R₆ are all absent.

In some of the variations of formula (a4), k is 2-10, Rₐ and R_{b} are either H or

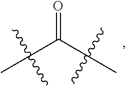 [SMILES: *C(*)=O]

and R₁ to R₆ are as described above for formula (4). In some of these variations, k is 6 or 8, Rₐ and R_{b} are either H or

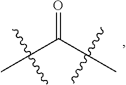 [SMILES: *C(*)=O]

and each of R₁ to R₆ independently is absent or is a halo. In particular variations, k is 6 or 8, Rₐ and R_{b} are either H or

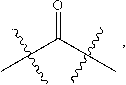 [SMILES: *C(*)=O]

and R₁ to R₆ are all absent.

In some of the variations of formula (4a), k is 2-10, each Rₐ and R_{b} is H or two of CRₐR_{b} are

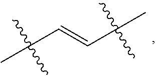 [SMILES: */C=C/*]

and R₁ to R₆ are as described above for formula (4). In some of these variations, k is 6 or 8, each Rₐ and R_{b} is H or two of CRₐR_{b} are

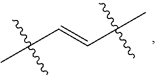 [SMILES: */C=C/*]

and each of R₁ to R₆ independently is absent or is a halo. In particular variations, k is 6 or 8, each Rₐ and R_{b} is H or two of CRₐR_{b} are

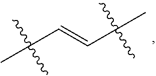 [SMILES: */C=C/*]

and R₁ to R₆ are all absent.

One of skill in the art can readily visualize and prepare other cationic multimers falling within formula (4) in which other cationic dye moieties are used in place of one or both of the safranin-O moieties.

Some cationic dye dimers fall within formula (5):

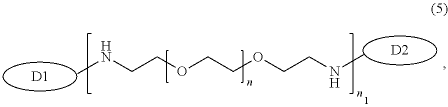

wherein each of D1 and D2 is a cationic dye moiety, n is 0-6, and n₁ is 1-4.

In some variations of formula (5), D1 and D2 are different cationic dye moieties. In other variations of formula (5), D1 and D2 are the same cationic dye moiety. In some variations of formula (5), D1 and D2 are independently selected from the group consisting of safranin-O, toluidine blue, azure A, azure B, azure C, acridine orange, acriflavine, and methylene blue.

In some variations of formula (5) described in the paragraphs above, n is 0-6, 0-5, 0-4, 0-3, 0-2, 0-1, 1-6, 1-5, 1-4, 1-3, 1-2, 2-6, 2-5, 2-4, 2-3, 3-6, 3-5, 3-4, 4-6, 4-5, 5-6, 0, 1, 2, 3, 4, 5, or 6.

In some variations of formula (5) described in the paragraphs above, n₁ is 1-4, 1-3, 1-2, 2-4, 2-3, 3-4, 1, 2, 3, or 4.

In some variations of formula (5) described in the paragraphs above, D1 is safranin-O. In some variations of formula (5) described in the paragraphs above, D2 is safranin-O. In some variations of formula (5) described in the paragraphs above, D1 and D2 are safranin-O.

In some variations of formula (5) described in the paragraph above, the pendant phenyl ring of D1 is unsubstituted. In some variations of formula (5) described in the paragraph above, the pendant phenyl ring of D1 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D1 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D1 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (5) described in the paragraph above, the pendant phenyl ring of D2 is unsubstituted. In some variations of formula (5) described in the paragraph above, the pendant phenyl ring of D2 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D2 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D2 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

Some cationic dye dimers fall within formula (6):

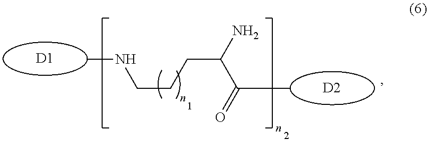

wherein each of D1 and D2 is a cationic dye moiety, n₁ is 0-5, and n₂ is 1-5.

In some variations of formula (6), D1 and D2 are different cationic dye moieties. In other variations of formula (6), D1 and D2 are the same cationic dye moiety. In some variations of formula (6), D1 and D2 are independently selected from the group consisting of safranin-O, toluidine blue, azure A, azure B, azure C, acridine orange, acriflavine, and methylene blue.

In some variations of formula (6) described in the paragraphs above, n₁ is 0-5, 0-4, 0-3, 0-2, 0-1, 1-5, 1-4, 1-3, 1-2, 2-5, 2-4, 2-3, 3-5, 3-4, 4-5, 0, 1, 2, 3, 4, or 5.

In some variations of formula (6) described in the paragraphs above, n₂ is 1-5, 1-4, 1-3, 1-2, 2-5, 2-4, 2-3, 3-5, 3-4, 4-5, 1, 2, 3, 4, or 5.

In some variations of formula (6) described in the paragraphs above, D1 is safranin-O. In some variations of formula (6) described in the paragraphs above, D2 is safranin-O. In some variations of formula (6) described in the paragraphs above, D1 and D2 are safranin-O.

In some variations of formula (6) described in the paragraph above, the pendant phenyl ring of D1 is unsubstituted. In some variations of formula (6) described in the paragraph above, the pendant phenyl ring of D1 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D1 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D1 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (6) described in the paragraph above, the pendant phenyl ring of D2 is unsubstituted. In some variations of formula (6) described in the paragraph above, the pendant phenyl ring of D2 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D2 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D2 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

Some cationic dye dimers fall within formula (7):

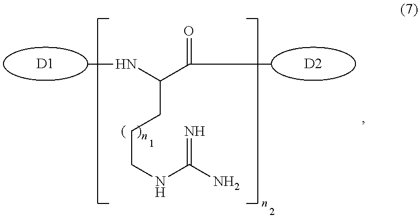

in which each of D1 and D2 is a cationic dye moiety, n₁ is 0-5, and n₂ is 1-5.

In some variations of formula (7), D1 and D2 are different cationic dye moieties. In other variations of formula (7), D1 and D2 are the same cationic dye moiety. In some variations of formula (7), D1 and D2 are independently selected from the group consisting of safranin-O, toluidine blue, azure A, azure B, azure C, acridine orange, acriflavine, and methylene blue.

In some variations of formula (7) described in the paragraphs above, n₁ is 0-5, 0-4, 0-3, 0-2, 0-1, 1-5, 1-4, 1-3, 1-2, 2-5, 2-4, 2-3, 3-5, 3-4, 4-5, 0, 1, 2, 3, 4, or 5.

In some variations of formula (7) described in the paragraphs above, n₂ is 1-5, 1-4, 1-3, 1-2, 2-5, 2-4, 2-3, 3-5, 3-4, 4-5, 1, 2, 3, 4, or 5.

In some variations of formula (7) described in the paragraphs above, D1 is safranin-O. In some variations of formula (7) described in the paragraphs above, D2 is safranin-O. In some variations of formula (7) described in the paragraphs above, D1 and D2 are safranin-O.

In some variations of formula (7) described in the paragraph above, the pendant phenyl ring of D1 is unsubstituted. In some variations of formula (7) described in the paragraph above, the pendant phenyl ring of D1 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D1 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D1 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (7) described in the paragraph above, the pendant phenyl ring of D2 is unsubstituted. In some variations of formula (7) described in the paragraph above, the pendant phenyl ring of D2 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D2 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D2 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

Some cationic dye dimers fall within formula (8):

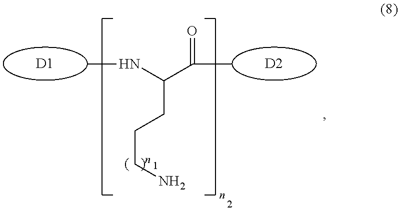

in which each of D1 and D2 is a cationic dye moiety, n₁ is 0-5, and n₂ is 1-5.

In some variations of formula (8), D1 and D2 are different cationic dye moieties. In other variations of formula (8), D1 and D2 are the same cationic dye moiety. In some variations of formula (8), D1 and D2 are independently selected from the group consisting of safranin-O, toluidine blue, azure A, azure B, azure C, acridine orange, acriflavine, and methylene blue.

In some variations of formula (8) described in the paragraphs above, n₁ is 0-5, 0-4, 0-3, 0-2, 0-1, 1-5, 1-4, 1-3, 1-2, 2-5, 2-4, 2-3, 3-5, 3-4, 4-5, 0, 1, 2, 3, 4, or 5.

In some variations of formula (8) described in the paragraphs above, n₂ is 1-5, 1-4, 1-3, 1-2, 2-5, 2-4, 2-3, 3-5, 3-4, 4-5, 1, 2, 3, 4, or 5.

In some variations of formula (8) described in the paragraphs above, D1 is safranin-O. In some variations of formula (8) described in the paragraphs above, D2 is safranin-O. In some variations of formula (8) described in the paragraphs above, D1 and D2 are safranin-O.

In some variations of formula (8) described in the paragraph above, the pendant phenyl ring of D1 is unsubstituted. In some variations of formula (8) described in the paragraph above, the pendant phenyl ring of D1 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D1 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D1 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (8) described in the paragraph above, the pendant phenyl ring of D2 is unsubstituted. In some variations of formula (8) described in the paragraph above, the pendant phenyl ring of D2 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D2 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D2 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

Some cationic dye dimers fall within formula (9):

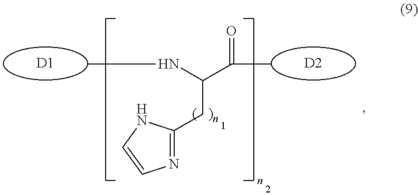

in which each of D1 and D2 is a cationic dye moiety and n₁ and n₂ independently are 1-5.

In some variations of formula (9), D1 and D2 are different cationic dye moieties. In other variations of formula (9), D1 and D2 are the same cationic dye moiety. In some variations of formula (9), D1 and D2 are independently selected from the group consisting of safranin-O, toluidine blue, azure A, azure B, azure C, acridine orange, acriflavine, and methylene blue.

In some variations of formula (9) described in the paragraphs above, n₁ is 1-5, 1-4, 1-3, 1-2, 2-5, 2-4, 2-3, 3-5, 3-4, 4-5, 1, 2, 3, 4, or 5.

In some variations of formula (9) described in the paragraphs above, n₂ is 1-5, 1-4, 1-3, 1-2, 2-5, 2-4, 2-3, 3-5, 3-4, 4-5, 1, 2, 3, 4, or 5.

In some variations of formula (9) described in the paragraphs above, D1 is safranin-O. In some variations of formula (9) described in the paragraphs above, D2 is safranin-O. In some variations of formula (9) described in the paragraphs above, D1 and D2 are safranin-O.

In some variations of formula (9) described in the paragraph above, the pendant phenyl ring of D1 is unsubstituted. In some variations of formula (9) described in the paragraph above, the pendant phenyl ring of D1 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D1 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D1 is substituted, the substituents are selected independently from —NO₂, —NR₃ halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (9) described in the paragraph above, the pendant phenyl ring of D2 is unsubstituted. In some variations of formula (9) described in the paragraph above, the pendant phenyl ring of D2 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D2 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D2 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

Some cationic dye multimers fall within formula (10):

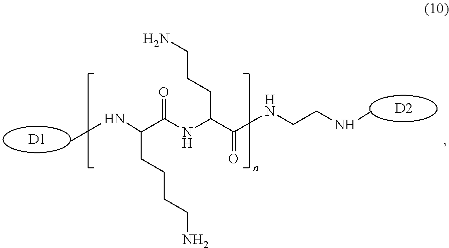

wherein each of D1 and D2 is a cationic dye moiety and n is 1-6.

In some variations of formula (10), D1 and D2 are different cationic dye moieties. In some variations of formula (10), D1 and D2 are the same cationic dye moiety. In some variations of formula (10), D1 and D2 independently are selected from the group consisting of safranin-O, toluidine blue, azure A, azure B, azure C, acridine orange, acriflavine, and methylene blue.

In some variations of formula (32) described above, n is 1-6, 1-5, 1-4, 1-3, 1-2, 2-6, 2-5, 12-4, 2-3, 3-6, 3-5, 3-4, 4-6, 4-5, 5-6, 1, 2, 3, 4, 5, or 6.

In some variations of formula (10) described in the paragraphs above, D1 is safranin-O. In some variations of formula (10) described in the paragraphs above, D2 is safranin-O. In some variations of formula (10) described in the paragraphs above, D1 and D2 are safranin-O.

In some variations of formula (10) described in the paragraph above, the pendant phenyl ring of D1 is unsubstituted. In some variations of formula (10) described in the paragraph above, the pendant phenyl ring of D1 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D1 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D1 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (10) described in the paragraph above, the pendant phenyl ring of D2 is unsubstituted. In some variations of formula (10) described in the paragraph above, the pendant phenyl ring of D2 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D2 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D2 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some of the variations of formula (10) described above, D1 and D2 are safranin-O moieties, as shown in formula (10a):

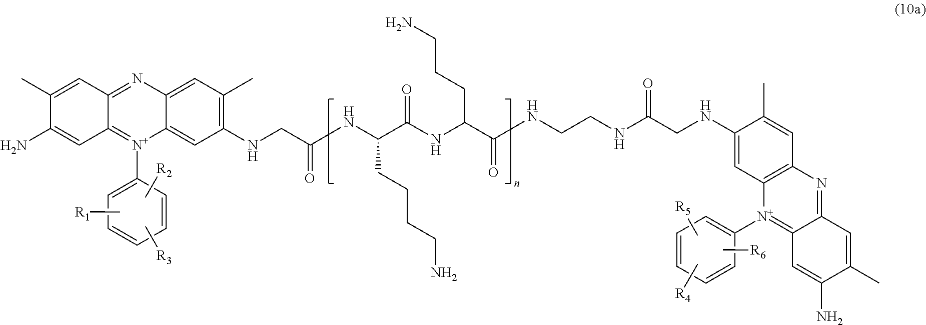

in which n is 1-6, R₁, R₂, R₃, R₄, R₅, and R₆ independently are absent or independently are selected from —NH₂, —NHR, —NR₂, —OH, —O⁻, —NHCOCH₃, —NHCOR, —OCH₃, —OR, —C₂H₅, —R, —C₆H₅, —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR), and R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (10a), n is 1-6, 1-5, 1-4, 1-3, 1-2, 2-6, 2-5, 2-4, 2-3, 3-6, 3-5, 3-4, 4-6, 4-5, 5-6, 1, 2, 3, 4, 5, or 6.

One of skill in the art can readily visualize and prepare other cationic multimers falling within formula (10) in which other cationic dye moieties are used in place of one or both of the safranin-O moieties.

Some cationic dye dimers fall within formula (11):

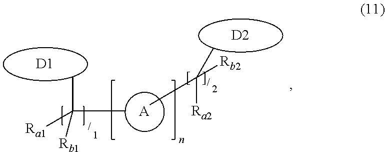

in which each of D1 and D2 is a cationic dye moiety; l₁ and l₂ independently are 1-4; n is 1-4; ring A is aryl, heteroaryl, cycloalkyl, or heterocyclyl; for each independent instance of Rₐ₁ and R_{b1}, Rₐ₁ and R_{b1} (1) independently are H or CH₃, or (2) Rₐ₁ and R_{b1} are

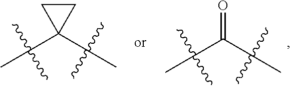

or (3) two of CRₐ₁R_{b1} are

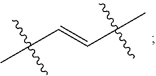 [SMILES: */C=C/*]

and, for each independent instance of Rₐ₂ and R_{b2}, Rₐ₂ and R_{b2} (1) independently are H or CH₃, or (2) Rₐ₂ and R_{b2} are

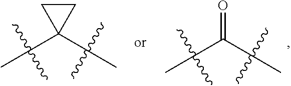

or (3) two of CRₐ₂R_{b2} are

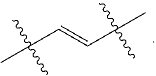 [SMILES: */C=C/*]

In some variations of formula (11), D1 and D2 are different cationic dye moieties. In other variations of formula (11), D1 and D2 are the same cationic dye moiety. In some variations of formula (11), D1 and D2 are independently selected from the group consisting of safranin-O, toluidine blue, azure A, azure B, azure C, acridine orange, acriflavine, and methylene blue.

In some variations of formula (11), l₁ is 1-4, 1-3, 1-2, 2-4, 2-3, 3-4, 1, 2, 3, or 4.

In some variations of formula (11) described in the paragraphs above, l₂ is 1-4, 1-3, 1-2, 2-4, 2-3, 3-4, 1, 2, 3, or 4.

In some variations of formula (11) described in the paragraphs above in which l₁ is 1, Rₐ₁ and R_{b1} are both H. In some variations of formula (11) described in the paragraphs above in which l₁ is 1, Rₐ₁ is H and R_{b1} is CH₃. In some variations of formula (11) described in the paragraphs above in which l₁ is 1, Rₐ₁ and R_{b1} are both CH₃. In some variations of formula (11) described in the paragraphs above in which l₁ is 1, Rₐ₁ and R_{b1} are

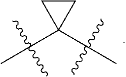 [SMILES: *C1(*)CC1]

In some variations of formula (11) described in the paragraphs above in which l₁ is 1, Rₐ₁ and R_{b1} are

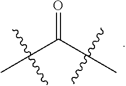 [SMILES: *C(*)=O]

In variations of formula (11) described in the paragraphs above in which l₁ is 2, the two instances of Rₐ and R_{b} are indicated as Rₐ₁ and R_{b1} and Rₐ₂ and R_{b2}, respectively. In some variations of formula (11) described in the paragraphs above in which l₁ is 2, each of Rₐ₁ and R_{b1} and Rₐ₂ and R_{b2} is H. In some variations of formula (11) described in the paragraphs above in which l₁ is 2, each of Rₐ₁ and R_{b1} and Rₐ₂ and R_{b2} is CH₃. In some variations of formula (11) described in the paragraphs above in which l₁ is 2, each of Rₐ₁ and Rₐ₂ is H and each of R_{b1} and R_{b2} is CH₃. In some variations of formula (11) described in the paragraphs above in which l₁ is 2, each of Rₐ₁, Rₐ₂, and R_{b1} is H and R_{b2} is CH₃.

In some variations of formula (11) described in the paragraphs above in which l₁ is 2, each of Rₐ₁ and R_{b1} is H and Rₐ₂ and R_{b2} are

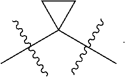 [SMILES: *C1(*)CC1]

In some variations of formula (11) described in the paragraphs above in which l₁ is 2, each of Rₐ₁ and R_{b1} is H and Rₐ₂ and R_{b2} are

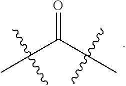 [SMILES: *C(*)=O]

In some variations of formula (11) described in the paragraphs above in which l₁ is 2, each of Rₐ₁ and R_{b1} is CH₃ and Rₐ₂ and R_{b2} are

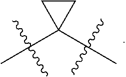 [SMILES: *C1(*)CC1]

In some variations of formula (11) described in the paragraphs above in which l₁ is 2, each of Rₐ₁ and R_{b1} is CH₃ and Rₐ₂ and R_{b2} are

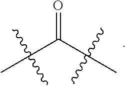 [SMILES: *C(*)=O]

In some variations of formula (11) described in the paragraphs above in which l₁ is 2, Rₐ₁ is H, R_{b1} is CH₃, and Rₐ₂ and R_{b2} are

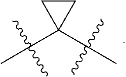 [SMILES: *C1(*)CC1]

In some variations of formula (11) described in the paragraphs above in which l₁ is 2, Rₐ₁ is H, R_{b1} is CH₃, and Rₐ₂ and R_{b2} are

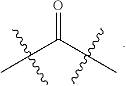 [SMILES: *C(*)=O]

In some variations of formula (11) described in the paragraphs above in which l₁ is 2, Rₐ₁ and R_{b1} and Rₐ₂ and R_{b2} together are

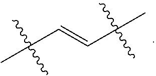 [SMILES: */C=C/*]

In some variations of formula (11) described in the paragraphs above in which l₁ is 2, Rₐ₁ and R_{b1} are

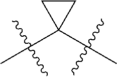 [SMILES: *C1(*)CC1]

Rₐ₂ and R_{b2} are

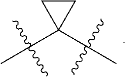 [SMILES: *C1(*)CC1]

In some variations of formula (11) described in the paragraphs above in which l₁ is 2, Rₐ₁ and R_{b1} are

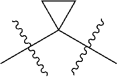 [SMILES: *C1(*)CC1]

Rₐ₂ and R_{b2} are

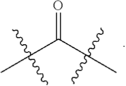 [SMILES: *C(*)=O]

In some variations of formula (11) described in the paragraphs above in which l₁ is 2, Rₐ₁ and R_{b1} are

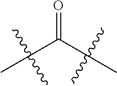 [SMILES: *C(*)=O]

Rₐ₂ and R_{b2} are

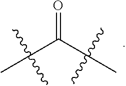 [SMILES: *C(*)=O]

In variations of formula (11) described in the paragraphs above in which l₁ is 3, the three instances of Rₐ and R_{b} are indicated as Rₐ₁ and R_{b1}; Rₐ₂ and R_{b2}; and Rₐ₃ and R_{b3}, respectively. In some variations of formula (11) described in the paragraphs above in which l₁ is 3, In some variations of formula (11) described in the paragraphs above in which l₁ is 3, each of Rₐ₁, R_{b1}, Rₐ₂, R_{b2}, Rₐ₃, and R_{b3} is H. In some variations of formula (11) described in the paragraphs above in which l₁ is 3, each of Rₐ₁, R_{b1}, Rₐ₂, R_{b2}, Rₐ₃, and R_{b3} is CH₃. In some variations of formula (11) described in the paragraphs above in which l₁ is 3, each of Rₐ₁, R_{b1}, Rₐ₂, an R_{b2}, is H and each of Rₐ₃ and R_{b3} is CH₃. In some variations of formula (11) described in the paragraphs above in which l₁ is 3, each of Rₐ₁, R_{b1}, Rₐ₂, and R_{b2}, is CH₃ and each of Rₐ₃ and R_{b3} is H. In some variations of formula (11) described in the paragraphs above in which l₁ is 3, Rₐ₁ is H and each of Rₐ₂, Rₐ₃, R_{b1}, R_{b2}, and R_{b3} is CH₃. In some variations of formula (11) described in the paragraphs above in which l₁ is 3, each of Rₐ₁ and Rₐ₂ is H and each of Rₐ₃, R_{b1}, R_{b2}, an R_{b3} is CH₃. In some variations of formula (11) described in the paragraphs above in which l₁ is 3, each of Rₐ₁, Rₐ₂, and Rₐ₃ is H and each of R_{b1}, R_{b2}, and R_{b3} is CH₃.

In some variations of formula (11) described in the paragraphs above in which l₁ is 3, each of Rₐ₁, R_{b1}, Rₐ₂, and R_{b2}, is H and Rₐ₃ and R_{b3} are

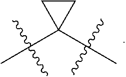 [SMILES: *C1(*)CC1]

In some variations of formula (11) described in the paragraphs above in which l₁ is 3, each of Rₐ₁, R_{b1}, Rₐ₂, and R_{b2}, is CH₃ and Rₐ₃ and R_{b3} are

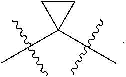 [SMILES: *C1(*)CC1]

In some variations of formula (11) described in the paragraphs above in which l₁ is 3, Rₐ₁ is H and each of Rₐ₂, R_{b1}, and R_{b2} is CH₃, and Rₐ₃ and R_{b3} are

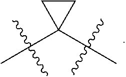 [SMILES: *C1(*)CC1]

In some variations of formula (11) described in the paragraphs above in which l₁ is 3, each of Rₐ₁ and Rₐ₂ is H and each of R_{b1} and R_{b2} is CH₃, and Rₐ₃ and R_{b3} are

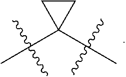 [SMILES: *C1(*)CC1]

In some variations of formula (11) described in the paragraphs above in which l₁ is 3, each of Rₐ₁, R_{b1}, Rₐ₂, and R_{b2}, is H and Rₐ₃ and R_{b3} are

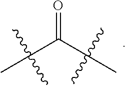 [SMILES: *C(*)=O]

In some variations of formula (11) described in the paragraphs above in which l₁ is 3, each of Rₐ₁, R_{b1}, Rₐ₂, and R_{b2}, is CH₃ and Rₐ₃ and R_{b3} are

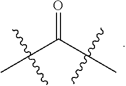 [SMILES: *C(*)=O]

In some variations of formula (11) described in the paragraphs above in which l₁ is 3, Rₐ₁ is H and each of Rₐ₂, R_{b1}, and R_{b2} is CH₃, and Rₐ₃ and R_{b3} are

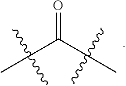 [SMILES: *C(*)=O]

In some variations of formula (11) described in the paragraphs above in which l₁ is 3, each of Rₐ₁ and Rₐ₂ is H and each of R_{b1} and R_{b2} is CH₃, and Rₐ₃ and R_{b3} are

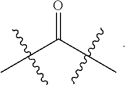 [SMILES: *C(*)=O]

In some variations of formula (11) described in the paragraphs above in which l₁ is 3, Rₐ₁ and R_{b1} are

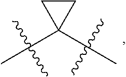 [SMILES: *C1(*)CC1]

Rₐ₂ and R_{b2} are

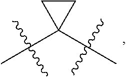 [SMILES: *C1(*)CC1]

and each of Rₐ₃ and R_{b3} is H. In some variations of formula (11) described in the paragraphs above in which l₁ is 3, Rₐ₁ and R_{b1} are

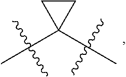 [SMILES: *C1(*)CC1]

Rₐ₂ and R_{b2} are

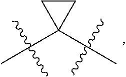 [SMILES: *C1(*)CC1]

and each of Rₐ₃ and R_{b3} is CH₃. In some variations of formula (11) described in the paragraphs above in which l₁ is 3, Rₐ₁ and R_{b1} are

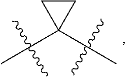 [SMILES: *C1(*)CC1]

Rₐ₂ and R_{b2} are

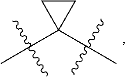 [SMILES: *C1(*)CC1]

Rₐ₃ is H, and R_{b3} is CH₃. In some variations of formula (11) described in the paragraphs above in which l₁ is 3, Rₐ₁ and R_{b1} are

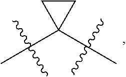 [SMILES: *C1(*)CC1]

Rₐ₂ and R_{b2} are

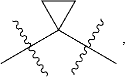 [SMILES: *C1(*)CC1]

and each of Rₐ₃ and R_{b3} is H. In some variations of formula (11) described in the paragraphs above in which l₁ is 3, Rₐ₁ and R_{b1} are

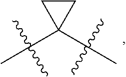 [SMILES: *C1(*)CC1]

Rₐ₂ and R_{b2} are

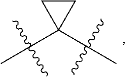 [SMILES: *C1(*)CC1]

and each of Rₐ₃ and R_{b3} is CH₃. In some variations of formula (11) described in the paragraphs above in which l₁ is 3, Rₐ₁ and R_{b1} are

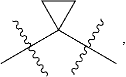 [SMILES: *C1(*)CC1]

Rₐ₂ and R_{b2} are

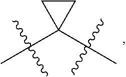 [SMILES: *C1(*)CC1]

Rₐ₃ is H, and R_{b3} is CH₃.

In some variations of formula (11) described in the paragraphs above in which l₁ is 3, Rₐ₁ and R_{b1} are

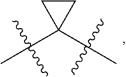 [SMILES: *C1(*)CC1]

Rₐ₂ and R_{b2} are

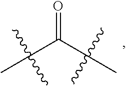 [SMILES: *C(*)=O]

and each of Rₐ₃ and R_{b3} is H. In some variations of formula (11) described in the paragraphs above in which l₁ is 3, Rₐ₁ and R_{b1} are

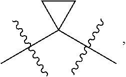 [SMILES: *C1(*)CC1]

Rₐ₂ and R_{b2} are

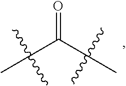 [SMILES: *C(*)=O]

and each of Rₐ₃ and R_{b3} is CH₃. In some variations of formula (11) described in the paragraphs above in which l₁ is 3, Rₐ₁ and R_{b1} are

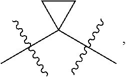 [SMILES: *C1(*)CC1]

Rₐ₂ and R_{b2} are

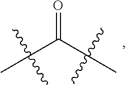 [SMILES: *C(*)=O]

Rₐ₃ is H, and R_{b3} is CH₃. In some variations of formula (11) described in the paragraphs above in which l₁ is 3, Rₐ₁ and R_{b1} are

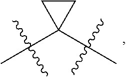 [SMILES: *C1(*)CC1]

Rₐ₂ and R_{b2} are

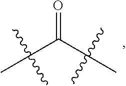 [SMILES: *C(*)=O]

and each of Rₐ₃ and R_{b3} is H. In some variations of formula (11) described in the paragraphs above in which l₁ is 3, Rₐ₁ and R_{b1} are

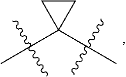 [SMILES: *C1(*)CC1]

Rₐ₂ and R_{b2} are

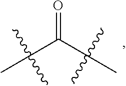 [SMILES: *C(*)=O]

and each of Rₐ₃ and R_{b3} is CH₃. In some variations of formula (11) described in the paragraphs above in which l₁ is 3, Rₐ₁ and R_{b1} are

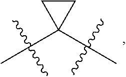 [SMILES: *C1(*)CC1]

Rₐ₂ and R_{b2} are

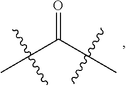 [SMILES: *C(*)=O]

Rₐ₃ is H, and R_{b3} is CH₃.

In some variations of formula (11) described in the paragraphs above in which l₁ is 3, Rₐ₁ and R_{b1} are

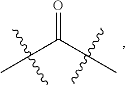 [SMILES: *C(*)=O]

Rₐ₂ and R_{b2} are

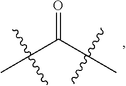 [SMILES: *C(*)=O]

and each of Rₐ₃ and R_{b3} is H. In some variations of formula (11) described in the paragraphs above in which l₁ is 3, Rₐ₁ and R_{b1} are

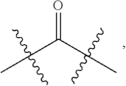 [SMILES: *C(*)=O]

Rₐ₂ and R_{b2} are

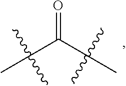 [SMILES: *C(*)=O]

and each of Rₐ₃ and R_{b3} is CH₃. In some variations of formula (11) described in the paragraphs above in which l₁ is 3, Rₐ₁ and R_{b1} are

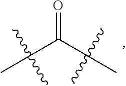 [SMILES: *C(*)=O]

Rₐ₂ and R_{b2} are

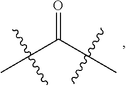 [SMILES: *C(*)=O]

Rₐ₃ is H, and R_{b3} is CH₃. In some variations of formula (11) described in the paragraphs above in which l₁ is 3, Rₐ₁ and R_{b1} are

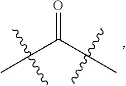 [SMILES: *C(*)=O]

Rₐ₂ and R_{b2} are

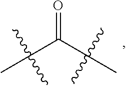 [SMILES: *C(*)=O]

and each of Rₐ₃ and R_{b3} is H. In some variations of formula (11) described in the paragraphs above in which l₁ is 3, Rₐ₁ and R_{b1} are

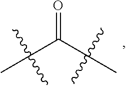 [SMILES: *C(*)=O]

Rₐ₂ and R_{b2} are

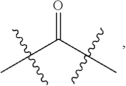 [SMILES: *C(*)=O]

and each of Rₐ₃ and R_{b3} is CH₃. In some variations of formula (11) described in the paragraphs above in which l₁ is 3, Rₐ₁ and R_{b1} are

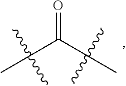 [SMILES: *C(*)=O]

Rₐ₂ and R_{b2} are

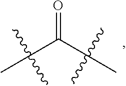 [SMILES: *C(*)=O]

Rₐ₃ is H, and R_{b3} is CH₃.

In some variations of formula (11) described in the paragraphs above in which l₁ is 3, Rₐ₁ and R_{b1} and Rₐ₂ and R_{b2} together are

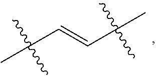 [SMILES: */C=C/*]

and each of Rₐ₃ and R_{b3} is H. In some variations of formula (11) described in the paragraphs above in which l₁ is 3, Rₐ₁ and R_{b1} and Rₐ₂ and R_{b2} together are

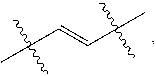 [SMILES: */C=C/*]

and each of Rₐ₃ and R_{b3} is CH₃. In some variations of formula (11) described in the paragraphs above in which l₁ is 3, Rₐ₁ and R_{b1} and Rₐ₂ and R_{b2} together are

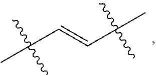 [SMILES: */C=C/*]

Rₐ₃ is H, and R_{b3} is CH₃. In some variations of formula (11) described in the paragraphs above in which l₁ is 3, Rₐ₁ and R_{b1} and Rₐ₂ and R_{b2} together are

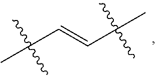 [SMILES: */C=C/*]

and Rₐ₃ and R_{b3} are

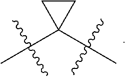 [SMILES: *C1(*)CC1]

In some variations of formula (11) described in the paragraphs above in which l₁ is 3, Rₐ₁ and R_{b1} and Rₐ₂ and R_{b2} together are

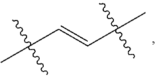 [SMILES: */C=C/*]

and Rₐ₃ and R_{b3} are

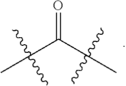 [SMILES: *C(*)=O]

In variations of formula (11) described in the paragraphs above in which l₁ is 4, the three instances of Rₐ and R_{b} are indicated as Rₐ₁ and R_{b1}; Rₐ₂ and R_{b2}; Rₐ₃ and R_{b3}; and Rₐ₄ and R_{b4}, respectively. In some variations of formula (11) described in the paragraphs above in which l₁ is 4, In some variations of formula (11) described in the paragraphs above in which l₁ is 4, each of Rₐ₁, R_{b1}, Rₐ₂, R_{b2}, Rₐ₃, R_{b3}, Rₐ₄, and R_{b4} is H. In some variations of formula (11) described in the paragraphs above in which l₁ is 4, each of Rₐ₁, R_{b1}, Rₐ₂, R_{b2}, Rₐ₃, R_{b3}, Rₐ₄, and R_{b4} is CH₃. In some variations of formula (11) described in the paragraphs above in which l₁ is 4, each of Rₐ₁, R_{b1}, Rₐ₂, R_{b2}, Rₐ₃, and R_{b3} is H and each of Rₐ₄ and R_{b4} is CH₃. In some variations of formula (11) described in the paragraphs above in which l₁ is 4, each of Rₐ₁, R_{b1}, Rₐ₂, and R_{b2} is H and each of Rₐ₃, R_{b3}, Rₐ₄, and R_{b4} is CH₃. In some variations of formula (11) described in the paragraphs above in which l₁ is 4, each of Rₐ₁, R_{b1}, Rₐ₂, R_{b2}, Rₐ₃, and R_{b3} is CH₃ and each of Rₐ₄ and R_{b4} is H.

In some variations of formula (11) described in the paragraphs above in which l₁ is 4, each of Rₐ₁, Rₐ₂, and Rₐ₃ is H and each of R_{b1}, R_{b2}, and R_{b3} is CH₃. In some variations of formula (11) described in the paragraphs above in which l₁ is 4, Rₐ₁ is H, R_{b1} is CH₃, and each of Rₐ₂, R_{b2}, Rₐ₃, and R_{b3} is CH₃. In some variations of formula (11) described in the paragraphs above in which l₁ is 4, Rₐ₁ is H, R_{b1} is CH₃, and each of Rₐ₂, R_{b2}, Rₐ₃ and R_{b3} is H. In some variations of formula (11) described in the paragraphs above in which l₁ is 4, each of Rₐ₁ and Rₐ₂ is H, each of R_{b1} and R_{b2} is CH₃, and each of Rₐ₃ and R_{b3} is H. In some variations of formula (11) described in the paragraphs above in which l₁ is 4, each of Rₐ₁ and Rₐ₂ is H, each of R_{b1} and R_{b2} is CH₃, and each of Rₐ₃ and R_{b3} is CH₃.

In some variations of formula (11) described in the paragraphs above in which l₁ is 4, Rₐ₁ and R_{b1} are H, Rₐ₂ and R_{b2} are H, Rₐ₃ and R_{b3} are H, and Rₐ₄ and R_{b4} are

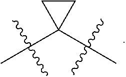 [SMILES: *C1(*)CC1]

In some variations of formula (11) described in the paragraphs above in which l₁ is 4, Rₐ₁ and R_{b1} are H, Rₐ₂ and R_{b2} are H, Rₐ₃ and R_{b3} are CH₃, and Rₐ₄ and R_{b4} are

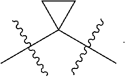 [SMILES: *C1(*)CC1]

In some variations of formula (11) described in the paragraphs above in which l₁ is 4, Rₐ₁ and R_{b1} are H, Rₐ₂ and R_{b2} are CH₃, Rₐ₃ and R_{b3} are CH₃, and Rₐ₄ and R_{b4} are

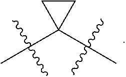 [SMILES: *C1(*)CC1]

In some variations of formula (11) described in the paragraphs above in which l₁ is 4, Rₐ₁ and R_{b1} are CH₃, Rₐ₂ and R_{b2} are CH₃, Rₐ₃ and R_{b3} are CH₃, and Rₐ₄ and R_{b4} are

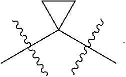 [SMILES: *C1(*)CC1]

In some variations of formula (11) described in the paragraphs above in which l₁ is 4, Rₐ₁ and R_{b1} are H, Rₐ₂ and R_{b2} are H, Rₐ₃ and R_{b3} are

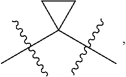 [SMILES: *C1(*)CC1]

and Rₐ₄ and R_{b4} are

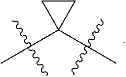 [SMILES: *C1(*)CC1]

In some variations of formula (11) described in the paragraphs above in which l₁ is 4, Rₐ₁ and R_{b1} are H, Rₐ₂ and R_{b2} are CH₃, Rₐ₃ and R_{b3} are

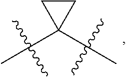 [SMILES: *C1(*)CC1]

and Rₐ₄ and R_{b4} are

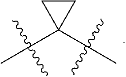 [SMILES: *C1(*)CC1]

In some variations of formula (11) described in the paragraphs above in which l₁ is 4, Rₐ₁ and R_{b1} are CH₃, Rₐ₂ and R_{b2} are CH₃, Rₐ₃ and R_{b3} are

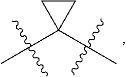 [SMILES: *C1(*)CC1]

and Rₐ₄ and R_{b4} are

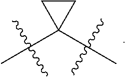 [SMILES: *C1(*)CC1]

In some variations of formula (11) described in the paragraphs above in which l₁ is 4, Rₐ₁ is H, R_{b1} is CH₃, Rₐ₂ and R_{b2} are CH₃, Rₐ₃ and R_{b3} are

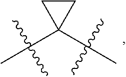 [SMILES: *C1(*)CC1]

and Rₐ₄ and R_{b4} are

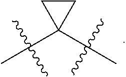 [SMILES: *C1(*)CC1]

In some variations of formula (11) described in the paragraphs above in which l₁ is 4, Rₐ₁ is H, R_{b1} is CH₃, Rₐ₂ is H, R_{b2} is CH₃, Rₐ₃ and R_{b3} are

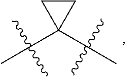 [SMILES: *C1(*)CC1]

and Rₐ₄ and R_{b4} are

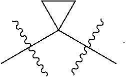 [SMILES: *C1(*)CC1]

In some variations of formula (11) described in the paragraphs above in which l₁ is 4, Rₐ₁ and R_{b1} are H, Rₐ₂ and R_{b2} are

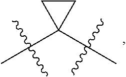 [SMILES: *C1(*)CC1]

Rₐ₃ and R_{b3} are

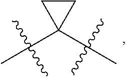 [SMILES: *C1(*)CC1]

and Rₐ₄ and R_{b4} are

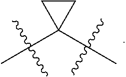 [SMILES: *C1(*)CC1]

In some variations of formula (11) described in the paragraphs above in which l₁ is 4, Rₐ₁ and R_{b1} are CH₃, Rₐ₂ and R_{b2} are

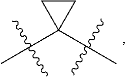 [SMILES: *C1(*)CC1]

Rₐ₃ and R_{b3} are

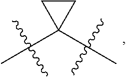 [SMILES: *C1(*)CC1]

and Rₐ₄ and R_{b4} are

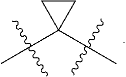 [SMILES: *C1(*)CC1]

In some variations of formula (11) described in the paragraphs above in which l₁ is 4, Rₐ₁ is H, R_{b1} is CH₃, Rₐ₂ and R_{b2} are

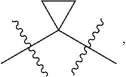 [SMILES: *C1(*)CC1]

Rₐ₃ and R_{b3} are

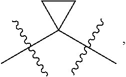 [SMILES: *C1(*)CC1]

and Rₐ₄ and R_{b4} are

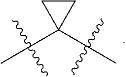 [SMILES: *C1(*)CC1]

In some variations of formula (11) described in the paragraphs above in which l₁ is 4, Rₐ₁ and R_{b1} are

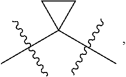 [SMILES: *C1(*)CC1]

Rₐ₂ and R_{b2} are

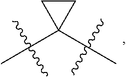 [SMILES: *C1(*)CC1]

Rₐ₃ and R_{b3} are

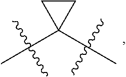 [SMILES: *C1(*)CC1]

and Rₐ₄ and R_{b4} are

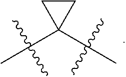 [SMILES: *C1(*)CC1]

In some variations of formula (11) described in the paragraphs above in which l₁ is 4, Rₐ₁ and R_{b1} are H, Rₐ₂ and R_{b2} are H, Rₐ₃ and R_{b3} are H, and Rₐ₄ and R_{b4} are

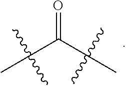 [SMILES: *C(*)=O]

In some variations of formula (11) described in the paragraphs above in which l₁ is 4, Rₐ₁ and R_{b1} are H, Rₐ₂ and R_{b2} are H, Rₐ₃ and R_{b3} are CH₃, and Rₐ₄ and R_{b4} are

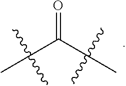 [SMILES: *C(*)=O]

In some variations of formula (11) described in the paragraphs above in which l₁ is 4, Rₐ₁ and R_{b1} are H, Rₐ₂ and R_{b2} are CH₃, Rₐ₃ and R_{b3} are CH₃, and Rₐ₄ and R_{b4} are

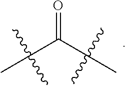 [SMILES: *C(*)=O]

In some variations of formula (11) described in the paragraphs above in which l₁ is 4, Rₐ₁ and R_{b1} are CH₃, Rₐ₂ and R_{b2} are CH₃, Rₐ₃ and R_{b3} are CH₃, and Rₐ₄ and R_{b4} are

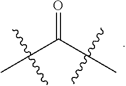 [SMILES: *C(*)=O]

In some variations of formula (11) described in the paragraphs above in which l₁ is 4, Rₐ₁ and R_{b1} are H, Rₐ₂ and R_{b2} are H, Rₐ₃ and R_{b3} are

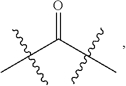 [SMILES: *C(*)=O]

and Rₐ₄ and R_{b4} are v. In some variations of formula (11) described in the paragraphs above in which l₁ is 4, Rₐ₁ and R_{b1} are H, Rₐ₂ and R_{b2} are CH₃, Rₐ₃ and R_{b3} are

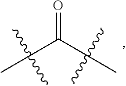 [SMILES: *C(*)=O]

and Rₐ₄ and R_{b4} are

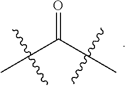 [SMILES: *C(*)=O]

In some variations of formula (11) described in the paragraphs above in which l₁ is 4, Rₐ₁ and R_{b1} are CH₃, Rₐ₂ and R_{b2} are CH₃, Rₐ₃ and R_{b3} are

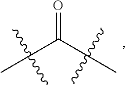 [SMILES: *C(*)=O]

and Rₐ₄ and R_{b4} are

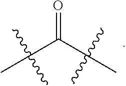 [SMILES: *C(*)=O]

In some variations of formula (11) described in the paragraphs above in which l₁ is 4, Rₐ₁ is H, R_{b1} is CH₃, Rₐ₂ and R_{b2} are CH₃, Rₐ₃ and R_{b3} are

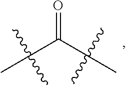 [SMILES: *C(*)=O]

and Rₐ₄ and R_{b4} are

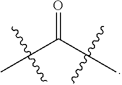 [SMILES: *C(*)=O]

In some variations of formula (11) described in the paragraphs above in which l₁ is 4, Rₐ₁ is H, R_{b1} is CH₃, Rₐ₂ is H, R_{b2} is CH₃, Rₐ₃ and R_{b3} are

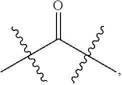 [SMILES: *C(*)=O]

and Rₐ₄ and R_{b4} are

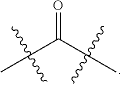 [SMILES: *C(*)=O]

In some variations of formula (11) described in the paragraphs above in which l₁ is 4, Rₐ₁ and R_{b1} are H, Rₐ₂ and R_{b2} are

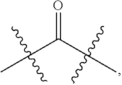 [SMILES: *C(*)=O]

Rₐ₃ and R_{b3} are

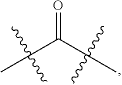 [SMILES: *C(*)=O]

and Rₐ₄ and R_{b4} are

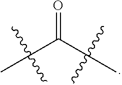 [SMILES: *C(*)=O]

In some variations of formula (11) described in the paragraphs above in which l₁ is 4, Rₐ₁ and R_{b1} are CH₃, Rₐ₂ and R_{b2} are

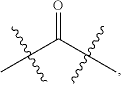 [SMILES: *C(*)=O]

Rₐ₃ and R_{b3} are

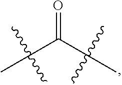 [SMILES: *C(*)=O]

and Rₐ₄ and R_{b4} are

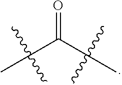 [SMILES: *C(*)=O]

In some variations of formula (11) described in the paragraphs above in which l₁ is 4, Rₐ₁ is H, R_{b1} is CH₃, Rₐ₂ and R_{b2} are

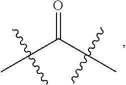 [SMILES: *C(*)=O]

Rₐ₃ and R_{b3} are

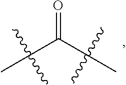 [SMILES: *C(*)=O]

and Rₐ₄ and R_{b4} are

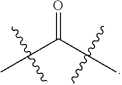 [SMILES: *C(*)=O]

In some variations of formula (11) described in the paragraphs above in which l₁ is 4, Rₐ₁ and R_{b1} are

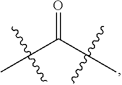 [SMILES: *C(*)=O]

Rₐ₂ and R_{b2} are

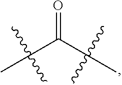 [SMILES: *C(*)=O]

Rₐ₃ and R_{b3} are

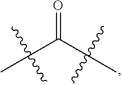 [SMILES: *C(*)=O]

and Rₐ₄ and R_{b4} are

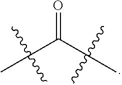 [SMILES: *C(*)=O]

In some variations of formula (11) described in the paragraphs above in which l₁ is 4, Rₐ₁ and R_{b1} are

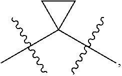 [SMILES: *C1(*)CC1]

Rₐ₂ and R_{b2} are H, Rₐ₃ and R_{b3} are H, and Rₐ₄ and R_{b4} are

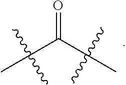 [SMILES: *C(*)=O]

In some variations of formula (11) described in the paragraphs above in which l₁ is 4, Rₐ₁ and R_{b1} are

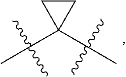 [SMILES: *C1(*)CC1]

Rₐ₂ and R_{b2} are CH₃, Rₐ₃ and R_{b3} are CH₃, and Rₐ₄ and R_{b4} are

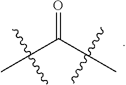 [SMILES: *C(*)=O]

In some variations of formula (11) described in the paragraphs above in which l₁ is 4, Rₐ₁ and R_{b1} are

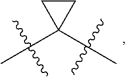 [SMILES: *C1(*)CC1]

Rₐ₂ and R_{b2} are H, Rₐ₃ and R_{b3} are CH₃, and Rₐ₄ and R_{b4} are

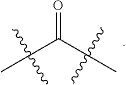 [SMILES: *C(*)=O]

In some variations of formula (11) described in the paragraphs above in which l₁ is 4, Rₐ₁ and R_{b1} are

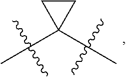 [SMILES: *C1(*)CC1]

Rₐ₂ and R_{b2} are

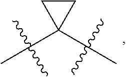 [SMILES: *C1(*)CC1]

Rₐ₃ and R_{b3} are H, and Rₐ₄ and R_{b4} are

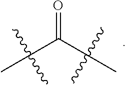 [SMILES: *C(*)=O]

In some variations of formula (11) described in the paragraphs above in which l₁ is 4, Rₐ₁ and R_{b1} are

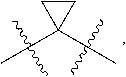 [SMILES: *C1(*)CC1]

Rₐ₂ and R_{b2} are

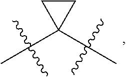 [SMILES: *C1(*)CC1]

Rₐ₃ and R_{b3} are CH₃, and Rₐ₄ and R_{b4} are

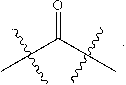 [SMILES: *C(*)=O]

In some variations of formula (11) described in the paragraphs above in which l₁ is 4, Rₐ₁ and R_{b1} are

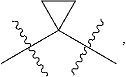 [SMILES: *C1(*)CC1]

Rₐ₂ and R_{b2} are

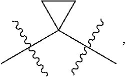 [SMILES: *C1(*)CC1]

Rₐ₃ and R_{b3} are CH₃, and Rₐ₄ and R_{b4} are

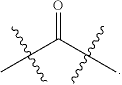 [SMILES: *C(*)=O]

In some variations of formula (11) described in the paragraphs above in which l₁ is 4, Rₐ₁ and R_{b1} are

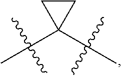 [SMILES: *C1(*)CC1]

Rₐ₂ and R_{b2} are

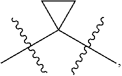 [SMILES: *C1(*)CC1]

Rₐ₃ and R_{b3} are H, and Rₐ₄ and R_{b4} are

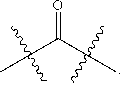 [SMILES: *C(*)=O]

In some variations of formula (11) described in the paragraphs above in which l₁ is 4, Rₐ₁ and R_{b1} are

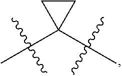 [SMILES: *C1(*)CC1]

Rₐ₂ and R_{b2} are

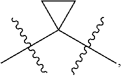 [SMILES: *C1(*)CC1]

Rₐ₃ and R_{b3} are

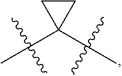 [SMILES: *C1(*)CC1]

and Rₐ₄ and R_{b4} are

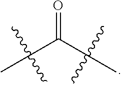 [SMILES: *C(*)=O]

In some variations of formula (11) described in the paragraphs above in which l₁ is 4, Rₐ₁ and R_{b1} are

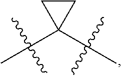 [SMILES: *C1(*)CC1]

Rₐ₂ and R_{b2} are

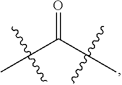 [SMILES: *C(*)=O]

Rₐ₃ and R_{b3} are H, and Rₐ₄ and R_{b4} are

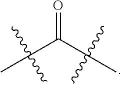 [SMILES: *C(*)=O]

In some variations of formula (11) described in the paragraphs above in which l₁ is 4, Rₐ₁ and R_{b1} are

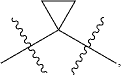 [SMILES: *C1(*)CC1]

Rₐ₂ and R_{b2} are

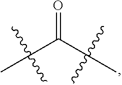 [SMILES: *C(*)=O]

Rₐ₃ and R_{b3} are CH₃, and Rₐ₄ and R_{b4} are

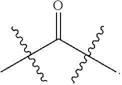 [SMILES: *C(*)=O]

In some variations of formula (11) described in the paragraphs above in which l₁ is 4, Rₐ₁ and R_{b1} are

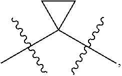 [SMILES: *C1(*)CC1]

Rₐ₂ and R_{b2} are

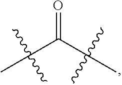 [SMILES: *C(*)=O]

Rₐ₃ and R_{b3} are CH₃, and Rₐ₄ and R_{b4} are

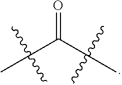 [SMILES: *C(*)=O]

In some variations of formula (11) described in the paragraphs above in which l₁ is 4, Rₐ₁ and R_{b1} are

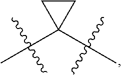 [SMILES: *C1(*)CC1]

Rₐ₂ and R_{b2} are

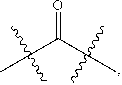 [SMILES: *C(*)=O]

Rₐ₃ and R_{b3} are H, and Rₐ₄ and R_{b4} are

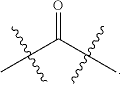 [SMILES: *C(*)=O]

In some variations of formula (11) described in the paragraphs above in which l₁ is 4, Rₐ₁ and R_{b1} are

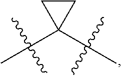 [SMILES: *C1(*)CC1]

Rₐ₂ and R_{b2} are

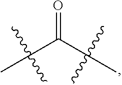 [SMILES: *C(*)=O]

Rₐ₃ and R_{b3} are

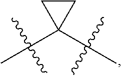 [SMILES: *C1(*)CC1]

and Rₐ₄ and R_{b4} are

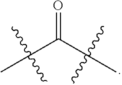 [SMILES: *C(*)=O]

In some variations of formula (11) described in the paragraphs above in which l₁ is 4, Rₐ₁ and R_{b1} are

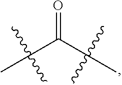 [SMILES: *C(*)=O]

Rₐ₂ and R_{b2} are

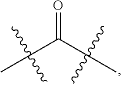 [SMILES: *C(*)=O]

Rₐ₃ and R_{b3} are H, and Rₐ₄ and R_{b4} are

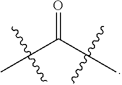 [SMILES: *C(*)=O]

In some variations of formula (11) described in the paragraphs above in which l₁ is 4, Rₐ₁ and R_{b1} are

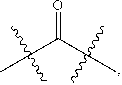 [SMILES: *C(*)=O]

Rₐ₂ and R_{b2} are

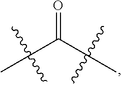 [SMILES: *C(*)=O]

Rₐ₃ and R_{b3} are CH₃, and Rₐ₄ and R_{b4} are

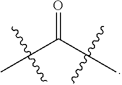 [SMILES: *C(*)=O]

In some variations of formula (11) described in the paragraphs above in which l₁ is 4, Rₐ₁ and R_{b1} are

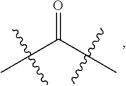 [SMILES: *C(*)=O]

Rₐ₂ and R_{b2} are

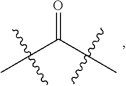 [SMILES: *C(*)=O]

Rₐ₃ and R_{b3} are CH₃, and Rₐ₄ and R_{b4} are

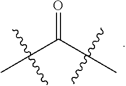 [SMILES: *C(*)=O]

In some variations of formula (11) described in the paragraphs above in which l₁ is 4, Rₐ₁ and R_{b1} are

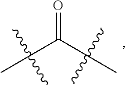 [SMILES: *C(*)=O]

Rₐ₂ and R_{b2} are

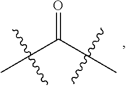 [SMILES: *C(*)=O]

Rₐ₃ and R_{b3} are H, and Rₐ₄ and R_{b4} are

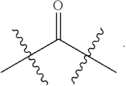 [SMILES: *C(*)=O]

In some variations of formula (11) described in the paragraphs above in which l₁ is 4, Rₐ₁ and R_{b1} are

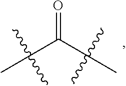 [SMILES: *C(*)=O]

Rₐ₂ and R_{b2} are

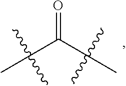 [SMILES: *C(*)=O]

Rₐ₃ and R_{b3} are

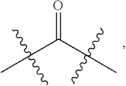 [SMILES: *C(*)=O]

and Rₐ₄ and R_{b4} are

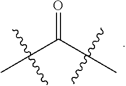 [SMILES: *C(*)=O]

In some variations of formula (11) described in the paragraphs above in which l₁ is 4, Rₐ₁ and R_{b1} are H, Rₐ₂ and R_{b2} are H, and Rₐ₃ and R_{b3} and Rₐ₄ and R_{b4} together are

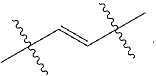 [SMILES: */C=C/*]

In some variations of formula (11) described in the paragraphs above in which l₁ is 4, Rₐ₁ and R_{b1} are H, Rₐ₂ and R_{b2} are CH₃, and Rₐ₃ and R_{b3} and Rₐ₄ and R_{b4} together are

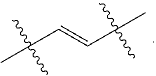 [SMILES: */C=C/*]

In some variations of formula (11) described in the paragraphs above in which l₁ is 4, Rₐ₁ and Rₐ₂ are H, R_{b1} and R_{b2} are CH₃, and Rₐ₃ and R_{b3} and Rₐ₄ and R_{b4} together are

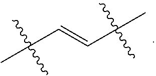 [SMILES: */C=C/*]

In some variations of formula (11) described in the paragraphs above in which l₁ is 4, Rₐ₁ and R_{b1} are H, Rₐ₂ and R_{b2} are

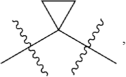 [SMILES: *C1(*)CC1]

and Rₐ₃ and R_{b3} and Rₐ₄ and R_{b4} together are

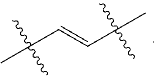 [SMILES: */C=C/*]

In some variations of formula (11) described in the paragraphs above in which l₁ is 4, Rₐ₁ and R_{b1} are

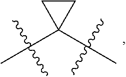 [SMILES: *C1(*)CC1]

Rₐ₂ and R_{b2} are CH₃, and Rₐ₃ and R_{b3} and Rₐ₄ and R_{b4} together are

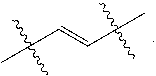 [SMILES: */C=C/*]

In some variations of formula (11) described in the paragraphs above in which l₁ is 4, Rₐ₁ is H, R_{b1} is CH₃, Rₐ₂ and R_{b2} are

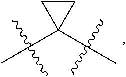 [SMILES: *C1(*)CC1]

and Rₐ₃ and R_{b3} and Rₐ₄ and R_{b4} together are

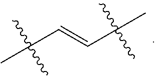 [SMILES: */C=C/*]

In some variations of formula (11) described in the paragraphs above in which l₁ is 4, Rₐ₁ and R_{b1} are H, Rₐ₂ and R_{b2} are

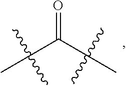 [SMILES: *C(*)=O]

and Rₐ₃ and R_{b3} and Rₐ₄ and R_{b4} together are

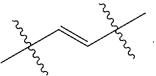 [SMILES: */C=C/*]

In some variations of formula (11) described in the paragraphs above in which l₁ is 4, Rₐ₁ and R_{b1} are

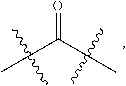 [SMILES: *C(*)=O]

Rₐ₂ and R_{b2} are CH₃, and Rₐ₃ and R_{b3} and Rₐ₄ and R_{b4} together are

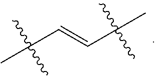 [SMILES: */C=C/*]

In some variations of formula (11) described in the paragraphs above in which l₁ is 4, Rₐ₁ is H, R_{b1} is CH₃, Rₐ₂ and R_{b2} are

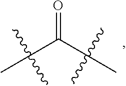 [SMILES: *C(*)=O]

and Rₐ₃ and R_{b3} and Rₐ₄ and R_{b4} together are

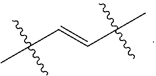 [SMILES: */C=C/*]

In some variations of formula (11) described in the paragraphs above in which l₁ is 4, Rₐ₁ and R_{b1} are

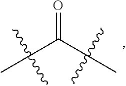 [SMILES: *C(*)=O]

Rₐ₂ and R_{b2} are

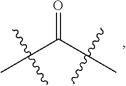 [SMILES: *C(*)=O]

and Rₐ₃ and R_{b3} and Rₐ₄ and R_{b4} together are

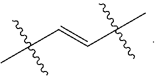 [SMILES: */C=C/*]

In some variations of formula (11) described in the paragraphs above in which l₁ is 4, Rₐ₁ and R_{b1} are

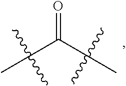 [SMILES: *C(*)=O]

Rₐ₂ and R_{b2} are

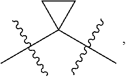 [SMILES: *C1(*)CC1]

and Rₐ₃ and R_{b3} and Rₐ₄ and R_{b4} together are

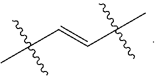 [SMILES: */C=C/*]

In some variations of formula (11) described in the paragraphs above in which l₁ is 4, Rₐ₁ and R_{b1} are

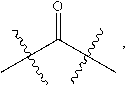 [SMILES: *C(*)=O]

Rₐ₂ and R_{b2} are

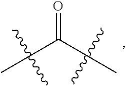 [SMILES: *C(*)=O]

and Rₐ₃ and R_{b3} and Rₐ₄ and R_{b4} together are

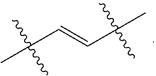 [SMILES: */C=C/*]

In some variations of formula (11) described in the paragraphs above in which l₁ is 4, Rₐ₁ and R_{b1} are

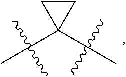 [SMILES: *C1(*)CC1]

Rₐ₂ and R_{b2} are

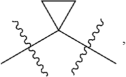 [SMILES: *C1(*)CC1]

and Rₐ₃ and R_{b3} and Rₐ₄ and R_{b4} together are

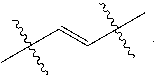 [SMILES: */C=C/*]

In some variations of formula (11) described in the paragraphs above in which l₁ is 4, Rₐ₁ and R_{b1} and Rₐ₂ and R_{b2} together are

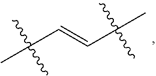 [SMILES: */C=C/*]

and Rₐ₃ and R_{b3} and Rₐ₄ and R_{b4} together are

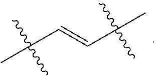 [SMILES: */C=C/*]

In some variations of formula (11) described in the paragraphs above in which l₂ is 1, Rₐ₁ and R_{b1} are both H. In some variations of formula (11) described in the paragraphs above in which l₂ is 1, Rₐ₁ is H and R_{b1} is CH₃. In some variations of formula (11) described in the paragraphs above in which l₂ is 1, Rₐ₁ and R_{b1} are both CH₃. In some variations of formula (11) described in the paragraphs above in which l₂ is 1, Rₐ₁ and R_{b1} are

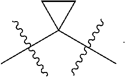 [SMILES: *C1(*)CC1]

In some variations of formula (11) described in the paragraphs above in which l₂ is 1, Rₐ₁ and R_{b1} are

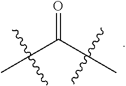 [SMILES: *C(*)=O]

In variations of formula (11) described in the paragraphs above in which l₂ is 2, the two instances of Rₐ and R_{b} are indicated as Rₐ₁ and R_{b1} and Rₐ₂ and R_{b2}, respectively. In some variations of formula (11) described in the paragraphs above in which l₂ is 2, each of Rₐ₁ and R_{b1} and Rₐ₂ and R_{b2} is H. In some variations of formula (11) described in the paragraphs above in which l₂ is 2, each of Rₐ₁ and R_{b1} and Rₐ₂ and R_{b2} is CH₃. In some variations of formula (11) described in the paragraphs above in which l₂ is 2, each of Rₐ₁ and Rₐ₂ is H and each of R_{b1} and R_{b2} is CH₃. In some variations of formula (11) described in the paragraphs above in which l₂ is 2, each of Rₐ₁, Rₐ₂, and R_{b1} is H and R_{b2} is CH₃.

In some variations of formula (11) described in the paragraphs above in which l₂ is 2, each of Rₐ₁ and R_{b1} is H and Rₐ₂ and R_{b2} are

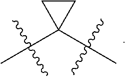 [SMILES: *C1(*)CC1]

In some variations of formula (11) described in the paragraphs above in which l₂ is 2, each of Rₐ₁ and R_{b1} is H and Rₐ₂ and R_{b2} are

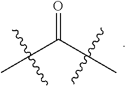 [SMILES: *C(*)=O]

In some variations of formula (11) described in the paragraphs above in which l₂ is 2, each of Rₐ₁ and R_{b1} is CH₃ and Rₐ₂ and R_{b2} are

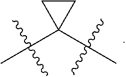 [SMILES: *C1(*)CC1]

In some variations of formula (11) described in the paragraphs above in which l₂ is 2, each of Rₐ₁ and R_{b1} is CH₃ and Rₐ₂ and R_{b2} are

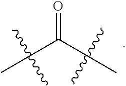 [SMILES: *C(*)=O]

In some variations of formula (11) described in the paragraphs above in which l₂ is 2, Rₐ₁ is H, R_{b1} is CH₃, and Rₐ₂ and R_{b2} are

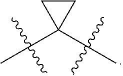 [SMILES: *C1(*)CC1]

In some variations of formula (11) described in the paragraphs above in which l₂ is 2, Rₐ₁ is H, R_{b1} is CH₃, and Rₐ₂ and R_{b2} are

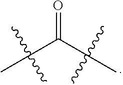 [SMILES: *C(*)=O]

In some variations of formula (11) described in the paragraphs above in which l₂ is 2, Rₐ₁ and R_{b1} and Rₐ₂ and R_{b2} together are

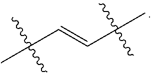 [SMILES: */C=C/*]

In some variations of formula (11) described in the paragraphs above in which l₂ is 2, Rₐ₁ and R_{b1} are

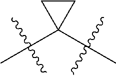 [SMILES: *C1(*)CC1]

Rₐ₂ and R_{b2} are

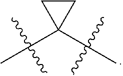 [SMILES: *C1(*)CC1]

In some variations of formula (11) described in the paragraphs above in which l₂ is 2, Rₐ₁ and R_{b1} are

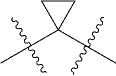 [SMILES: *C1(*)CC1]

Rₐ₂ and R_{b2} are

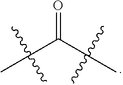 [SMILES: *C(*)=O]

In some variations of formula (11) described in the paragraphs above in which l₂ is 2, Rₐ₁ and R_{b1} are

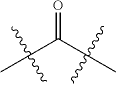 [SMILES: *C(*)=O]

Rₐ₂ and R_{b2} are

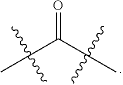 [SMILES: *C(*)=O]

In variations of formula (11) described in the paragraphs above in which l₂ is 3, the three instances of Rₐ and R_{b} are indicated as Rₐ₁ and R_{b1}; Rₐ₂ and R_{b2}; and Rₐ₃ and R_{b3}, respectively. In some variations of formula (11) described in the paragraphs above in which l₂ is 3, In some variations of formula (11) described in the paragraphs above in which l₂ is 3, each of Rₐ₁, R_{b1}, Rₐ₂, R_{b2}, Rₐ₃, and R_{b3} is H. In some variations of formula (11) described in the paragraphs above in which l₂ is 3, each of Rₐ₁, R_{b1}, Rₐ₂, R_{b2}, Rₐ₃, and R_{b3} is CH₃. In some variations of formula (11) described in the paragraphs above in which l₂ is 3, each of Rₐ₁, R_{b1}, Rₐ₂, an R_{b2}, is H and each of Rₐ₃ and R_{b3} is CH₃. In some variations of formula (11) described in the paragraphs above in which l₂ is 3, each of Rₐ₁, R_{b1}, Rₐ₂, and R_{b2}, is CH₃ and each of Rₐ₃ and R_{b3} is H. In some variations of formula (11) described in the paragraphs above in which l₂ is 3, Rₐ₁ is H and each of Rₐ₂, Rₐ₃, R_{b1}, R_{b2}, and R_{b3} is CH₃. In some variations of formula (11) described in the paragraphs above in which l₂ is 3, each of Rₐ₁ and Rₐ₂ is H and each of Rₐ₃, R_{b1}, R_{b2}, an R_{b3} is CH₃. In some variations of formula (11) described in the paragraphs above in which l₂ is 3, each of Rₐ₁, Rₐₑ, and Rₐ₃ is H and each of R_{b1}, R_{b2}, and R_{b3} is CH₃.

In some variations of formula (11) described in the paragraphs above in which l₂ is 3, each of Rₐ₁, R_{b1}, Rₐₑ, and R_{b2}, is H and Rₐ₃ and R_{b3} are

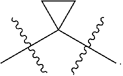 [SMILES: *C1(*)CC1]

In some variations of formula (11) described in the paragraphs above in which l₂ is 3, each of Rₐ₁, R_{b1}, Rₐ₂, and R_{b2}, is CH₃ and Rₐ₃ and R_{b3} are

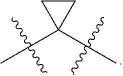 [SMILES: *C1(*)CC1]

In some variations of formula (11) described in the paragraphs above in which l₂ is 3, Rₐ₁ is H and each of Rₐ₂, R_{b1}, and R_{b2} is CH₃, and Rₐ₃ and R_{b3} are

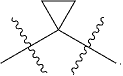 [SMILES: *C1(*)CC1]

In some variations of formula (11) described in the paragraphs above in which l₂ is 3, each of Rₐ₁ and Rₐ₂ is H and each of R_{b1} and R_{b2} is CH₃, and Rₐ₃ and R_{b3} are

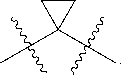 [SMILES: *C1(*)CC1]

In some variations of formula (11) described in the paragraphs above in which l₂ is 3, each of Rₐ₁, R_{b1}, Rₐₑ, and R_{b2}, is H and Rₐ₃ and R_{b3} are

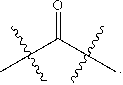 [SMILES: *C(*)=O]

In some variations of formula (11) described in the paragraphs above in which l₂ is 3, each of Rₐ₁, R_{b1}, Rₐ₂, and R_{b2}, is CH₃ and Rₐ₃ and R_{b3} are

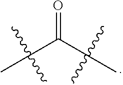 [SMILES: *C(*)=O]

In some variations of formula (11) described in the paragraphs above in which l₂ is 3, Rₐ₁ is H and each of Rₐ₂, R_{b1}, and R_{b2} is CH₃, and Rₐ₃ and R_{b3} are

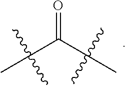 [SMILES: *C(*)=O]

In some variations of formula (11) described in the paragraphs above in which l₂ is 3, each of Rₐ₁ and Rₐ₂ is H and each of R_{b1} and R_{b2} is CH₃, and Rₐ₃ and R_{b3} are

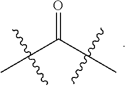 [SMILES: *C(*)=O]

In some variations of formula (11) described in the paragraphs above in which l₂ is 3, Rₐ₁ and R_{b1} are

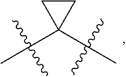 [SMILES: *C1(*)CC1]

Rₐ₂ and R_{b2} are

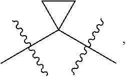 [SMILES: *C1(*)CC1]

and each of Rₐ₃ and R_{b3} is H. In some variations of formula (11) described in the paragraphs above in which l₂ is 3, Rₐ₁ and R_{b1} are

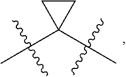 [SMILES: *C1(*)CC1]

Rₐ₂ and R_{b2} are

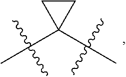 [SMILES: *C1(*)CC1]

and each of Rₐ₃ and R_{b3} is CH₃. In some variations of formula (11) described in the paragraphs above in which l₂ is 3, Rₐ₁ and R_{b1} are

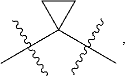 [SMILES: *C1(*)CC1]

Rₐ₂ and R_{b2} are

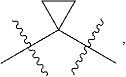 [SMILES: *C1(*)CC1]

Rₐ₃ is H, and R_{b3} is CH₃. In some variations of formula (11) described in the paragraphs above in which l₂ is 3, Rₐ₁ and R_{b1} are

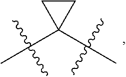 [SMILES: *C1(*)CC1]

Rₐ₂ and R_{b2} are

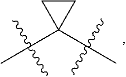 [SMILES: *C1(*)CC1]

and each of Rₐ₃ and R_{b3} is H. In some variations of formula (11) described in the paragraphs above in which l₂ is 3, Rₐ₁ and R_{b1} are

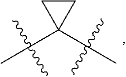 [SMILES: *C1(*)CC1]

Rₐ₂ and R_{b2} are

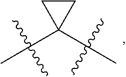 [SMILES: *C1(*)CC1]

and each of Rₐ₃ and R_{b3} is CH₃. In some variations of formula (11) described in the paragraphs above in which l₂ is 3, Rₐ₁ and R_{b1} are

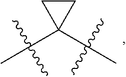 [SMILES: *C1(*)CC1]

Rₐ₂ and R_{b2} are

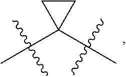 [SMILES: *C1(*)CC1]

Rₐ₃ is H, and R_{b3} is CH₃.

In some variations of formula (11) described in the paragraphs above in which l₂ is 3, Rₐ₁ and R_{b1} are

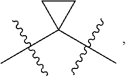 [SMILES: *C1(*)CC1]

Rₐ₂ and R_{b2} are

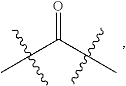 [SMILES: *C(*)=O]

and each of Rₐ₃ and R_{b3} is H. In some variations of formula (11) described in the paragraphs above in which l₂ is 3, Rₐ₁ and R_{b1} are

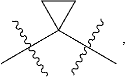 [SMILES: *C1(*)CC1]

Rₐ₂ and R_{b2} are

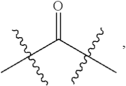 [SMILES: *C(*)=O]

and each of Rₐ₃ and R_{b3} is CH₃. In some variations of formula (11) described in the paragraphs above in which l₂ is 3, Rₐ₁ and R_{b1} are

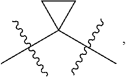 [SMILES: *C1(*)CC1]

Rₐ₂ and R_{b2} are

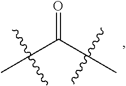 [SMILES: *C(*)=O]

Rₐ₃ is H, and R_{b3} is CH₃. In some variations of formula (11) described in the paragraphs above in which l₂ is 3, Rₐ₁ and R_{b1} are

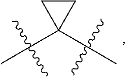 [SMILES: *C1(*)CC1]

Rₐ₂ and R_{b2} are

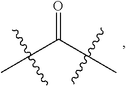 [SMILES: *C(*)=O]

and each of Rₐ₃ and R_{b3} is H. In some variations of formula (11) described in the paragraphs above in which l₂ is 3, Rₐ₁ and R_{b1} are

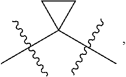 [SMILES: *C1(*)CC1]

Rₐ₂ and R_{b2} are

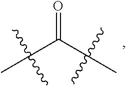 [SMILES: *C(*)=O]

and each of Rₐ₃ and R_{b3} is CH₃. In some variations of formula (11) described in the paragraphs above in which l₂ is 3, Rₐ₁ and R_{b1} are

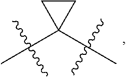 [SMILES: *C1(*)CC1]

Rₐ₂ and R_{b2} are

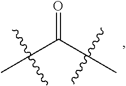 [SMILES: *C(*)=O]

Rₐ₃ is H, and R_{b3} is CH₃.

In some variations of formula (11) described in the paragraphs above in which l₂ is 3, Rₐ₁ and R_{b1} are

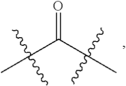 [SMILES: *C(*)=O]

Rₐ₂ and R_{b2} are

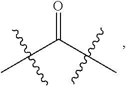 [SMILES: *C(*)=O]

and each of Rₐ₃ and R_{b3} is H. In some variations of formula (11) described in the paragraphs above in which l₂ is 3, Rₐ₁ and R_{b1} are

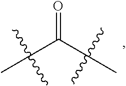 [SMILES: *C(*)=O]

Rₐ₂ and R_{b2} are

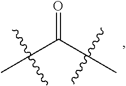 [SMILES: *C(*)=O]

and each of Rₐ₃ and R_{b3} is CH₃. In some variations of formula (11) described in the paragraphs above in which l₂ is 3, Rₐ₁ and R_{b1} are

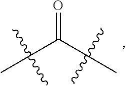 [SMILES: *C(*)=O]

Rₐ₂ and R_{b2} are

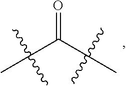 [SMILES: *C(*)=O]

Rₐ₃ is H, and R_{b3} is CH₃. In some variations of formula (11) described in the paragraphs above in which l₂ is 3, Rₐ₁ and R_{b1} are

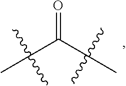 [SMILES: *C(*)=O]

Rₐ₂ and R_{b2} are

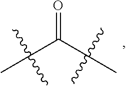 [SMILES: *C(*)=O]

and each of Rₐ₃ and R_{b3} is H. In some variations of formula (11) described in the paragraphs above in which l₂ is 3, Rₐ₁ and R_{b1} are

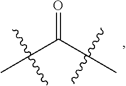 [SMILES: *C(*)=O]

Rₐ₂ and R_{b2} are

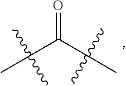 [SMILES: *C(*)=O]

and each of Rₐ₃ and R_{b3} is CH₃. In some variations of formula (11) described in the paragraphs above in which l₂ is 3, Rₐ₁ and R_{b1} are

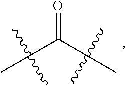 [SMILES: *C(*)=O]

Rₐ₂ and R_{b2} are

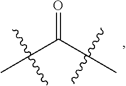 [SMILES: *C(*)=O]

Rₐ₃ is H, and R_{b3} is CH₃.

In some variations of formula (11) described in the paragraphs above in which l₂ is 3, Rₐ₁ and R_{b1} and Rₐ₂ and R_{b2} together are

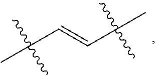 [SMILES: */C=C/*]

and each of Rₐ₃ and R_{b3} is H. In some variations of formula (11) described in the paragraphs above in which l₂ is 3, Rₐ₁ and R_{b1} and Rₐ₂ and R_{b2} together are

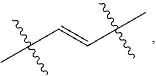 [SMILES: */C=C/*]

and each of Rₐ₃ and R_{b3} is CH₃. In some variations of formula (11) described in the paragraphs above in which l₂ is 3, Rₐ₁ and R_{b1} and Rₐ₂ and R_{b2} together are

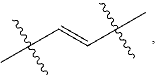 [SMILES: */C=C/*]

Rₐ₃ is H, and R_{b3} is CH₃. In some variations of formula (11) described in the paragraphs above in which l₂ is 3, Rₐ₁ and R_{b1} and Rₐ₂ and R_{b2} together are

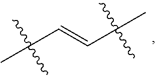 [SMILES: */C=C/*]

and Rₐ₃ and R_{b3} are

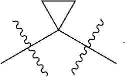 [SMILES: *C1(*)CC1]

In some variations of formula (11) described in the paragraphs above in which l₂ is 3, Rₐ₁ and R_{b1} and Rₐ₂ and R_{b2} together are

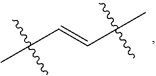 [SMILES: */C=C/*]

and Rₐ₃ and R_{b3} are

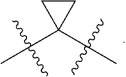 [SMILES: *C1(*)CC1]

In variations of formula (11) described in the paragraphs above in which l₂ is 4, the three instances of Rₐ and R_{b} are indicated as Rₐ₁ and R_{b1}; Rₐ₂ and R_{b2}; Rₐ₃ and R_{b3}; and Rₐ₄ and R_{b4}, respectively. In some variations of formula (11) described in the paragraphs above in which l₂ is 4, In some variations of formula (11) described in the paragraphs above in which l₂ is 4, each of Rₐ₁, R_{b1}, Rₐ₂, R_{b2}, Rₐ₃, R_{b3}, Rₐ₄, and R_{b4} is H. In some variations of formula (11) described in the paragraphs above in which l₂ is 4, each of Rₐ₁, R_{b1}, Rₐ₂, R_{b2}, Rₐ₃, R_{b3}, Rₐ₄, and R_{b4} is CH₃. In some variations of formula (11) described in the paragraphs above in which l₂ is 4, each of Rₐ₁, R_{b1}, Rₐ₂, R_{b2}, Rₐ₃, and R_{b3} is H and each of Rₐ₄ and R_{b4} is CH₃. In some variations of formula (11) described in the paragraphs above in which l₂ is 4, each of Rₐ₁, R_{b1}, Rₐ₂, and R_{b2} is H and each of Rₐ₃, R_{b3}, Rₐ₄, and R_{b4} is CH₃. In some variations of formula (11) described in the paragraphs above in which l₂ is 4, each of Rₐ₁, R_{b1}, Rₐ₂, R_{b2}, Rₐ₃, and R_{b3} is CH₃ and each of Rₐ₄ and R_{b4} is H.

In some variations of formula (11) described in the paragraphs above in which l₂ is 4, each of Rₐ₁, Rₐ₂, and Rₐ₃ is H and each of R_{b1}, R_{b2}, and R_{b3} is CH₃. In some variations of formula (11) described in the paragraphs above in which l₂ is 4, Rₐ₁ is H, R_{b1} is CH₃, and each of Rₐ₂, R_{b2}, Rₐ₃, and R_{b3} is CH₃. In some variations of formula (11) described in the paragraphs above in which l₂ is 4, Rₐ₁ is H, R_{b1} is CH₃, and each of Rₐ₂, R_{b2}, Rₐ₃, and R_{b3} is H. In some variations of formula (11) described in the paragraphs above in which l₂ is 4, each of Rₐ₁ and Rₐ₂ is H, each of R_{b1} and R_{b2} is CH₃, and each of Rₐ₃ and R_{b3} is H. In some variations of formula (11) described in the paragraphs above in which l₂ is 4, each of Rₐ₁ and Rₐ₂ is H, each of R_{b1} and R_{b2} is CH₃, and each of Rₐ₃ and R_{b3} is CH₃.

In some variations of formula (11) described in the paragraphs above in which l₂ is 4, Rₐ₁ and R_{b1} are H, Rₐ₂ and R_{b2} are H, Rₐ₃ and R_{b3} are H, and Rₐ₄ and R_{b4} are

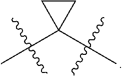 [SMILES: *C1(*)CC1]

In some variations of formula (11) described in the paragraphs above in which l₂ is 4, Rₐ₁ and R_{b1} are H, Rₐ₂ and R_{b2} are H, Rₐ₃ and R_{b3} are CH₃, and Rₐ₄ and R_{b4} are

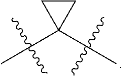 [SMILES: *C1(*)CC1]

In some variations of formula (11) described in the paragraphs above in which l₂ is 4, Rₐ₁ and R_{b1} are H, Rₐ₂ and R_{b2} are CH₃, Rₐ₃ and R_{b3} are CH₃, and Rₐ₄ and R_{b4} are

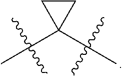 [SMILES: *C1(*)CC1]

In some variations of formula (11) described in the paragraphs above in which l₂ is 4, Rₐ₁ and R_{b1} are CH₃, Rₐ₂ and R_{b2} are CH₃, Rₐ₃ and R_{b3} are CH₃, and Rₐ₄ and R_{b4} are

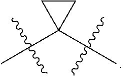 [SMILES: *C1(*)CC1]

In some variations of formula (11) described in the paragraphs above in which l₂ is 4, Rₐ₁ and R_{b1} are H, Rₐ₂ and R_{b2} are H, Rₐ₃ and R_{b3} are

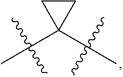 [SMILES: *C1(*)CC1]

and Rₐ₄ and R_{b4} are

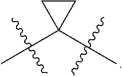 [SMILES: *C1(*)CC1]

In some variations of formula (11) described in the paragraphs above in which l₂ is 4, Rₐ₁ and R_{b1} are H, Rₐ₂ and R_{b2} are CH₃, Rₐ₃ and R_{b3} are

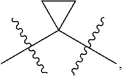 [SMILES: *C1(*)CC1]

and Rₐ₄ and R_{b4} are

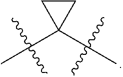 [SMILES: *C1(*)CC1]

In some variations of formula (11) described in the paragraphs above in which l₂ is 4, Rₐ₁ and R_{b1} are CH₃, Rₐ₂ and R_{b2} are CH₃, Rₐ₃ and R_{b3} are

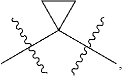 [SMILES: *C1(*)CC1]

and Rₐ₄ and R_{b4} are

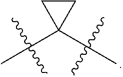 [SMILES: *C1(*)CC1]

In some variations of formula (11) described in the paragraphs above in which l₂ is 4, Rₐ₁ is H, R_{b1} is CH₃, Rₐ₂ and R_{b2} are CH₃, Rₐ₃ and R_{b3} are

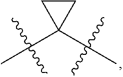 [SMILES: *C1(*)CC1]

and Rₐ₄ and R_{b4} are

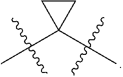 [SMILES: *C1(*)CC1]

In some variations of formula (11) described in the paragraphs above in which l₂ is 4, Rₐ₁ is H, R_{b1} is CH₃, Rₐ₂ is H, R_{b2} is CH₃, Rₐ₃ and R_{b3} are

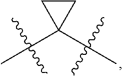 [SMILES: *C1(*)CC1]

and Rₐ₄ and R_{b4} are

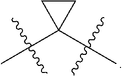 [SMILES: *C1(*)CC1]

In some variations of formula (11) described in the paragraphs above in which l₂ is 4, Rₐ₁ and R_{b1} are H, Rₐ₂ and R_{b2} are

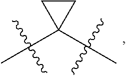 [SMILES: *C1(*)CC1]

Rₐ₃ and R_{b3} are

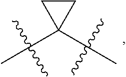 [SMILES: *C1(*)CC1]

and Rₐ₄ and R_{b4} are

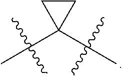 [SMILES: *C1(*)CC1]

In some variations of formula (11) described in the paragraphs above in which l₂ is 4, Rₐ₁ and R_{b1} are CH₃, Rₐ₂ and R_{b2} are

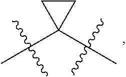 [SMILES: *C1(*)CC1]

Rₐ₃ and R_{b3} are

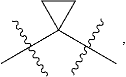 [SMILES: *C1(*)CC1]

and Rₐ₄ and R_{b4} are

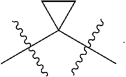 [SMILES: *C1(*)CC1]

In some variations of formula (11) described in the paragraphs above in which l₂ is 4, Rₐ₁ is H, R_{b1} is CH₃, Rₐ₂ and R_{b2} are

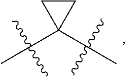 [SMILES: *C1(*)CC1]

Rₐ₃ and R_{b3} are

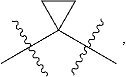 [SMILES: *C1(*)CC1]

and Rₐ₄ and R_{b4} are

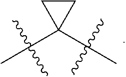 [SMILES: *C1(*)CC1]

In some variations of formula (11) described in the paragraphs above in which l₂ is 4, Rₐ₁ and R_{b1} are

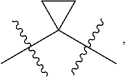 [SMILES: *C1(*)CC1]

Rₐ₂ and R_{b2} are

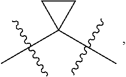 [SMILES: *C1(*)CC1]

Rₐ₃ and R_{b3} are

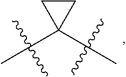 [SMILES: *C1(*)CC1]

and Rₐ₄ and R_{b4} are

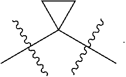 [SMILES: *C1(*)CC1]

In some variations of formula (11) described in the paragraphs above in which l₂ is 4, Rₐ₁ and R_{b1} are H, Rₐ₂ and R_{b2} are H, Rₐ₃ and R_{b3} are H, and Rₐ₄ and R_{b4} are

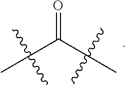 [SMILES: *C(*)=O]

In some variations of formula (11) described in the paragraphs above in which l₂ is 4, Rₐ₁ and R_{b1} are H, Rₐ₂ and R_{b2} are H, Rₐ₃ and R_{b3} are CH₃, and Rₐ₄ and R_{b4} are

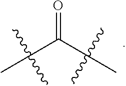 [SMILES: *C(*)=O]

In some variations of formula (11) described in the paragraphs above in which l₂ is 4, Rₐ₁ and R_{b1} are H, Rₐ₂ and R_{b2} are CH₃, Rₐ₃ and R_{b3} are CH₃, and Rₐ₄ and R_{b4} are

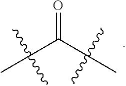 [SMILES: *C(*)=O]

In some variations of formula (11) described in the paragraphs above in which l₂ is 4, Rₐ₁ and R_{b1} are CH₃, Rₐ₂ and R_{b2} are CH₃, Rₐ₃ and R_{b3} are CH₃, and Rₐ₄ and R_{b4} are

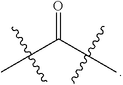 [SMILES: *C(*)=O]

In some variations of formula (11) described in the paragraphs above in which l₂ is 4, Rₐ₁ and R_{b1} are H, Rₐ₂ and R_{b2} are H, Rₐ₃ and R_{b3} are

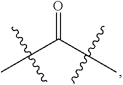 [SMILES: *C(*)=O]

and Rₐ₄ and R_{b4} are v. In some variations of formula (11) described in the paragraphs above in which l₂ is 4, Rₐ₁ and R_{b1} are H, Rₐ₂ and R_{b2} are CH₃, Rₐ₃ and R_{b3} are

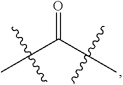 [SMILES: *C(*)=O]

and Rₐ₄ and R_{b4} are

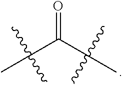 [SMILES: *C(*)=O]

In some variations of formula (11) described in the paragraphs above in which l₂ is 4, Rₐ₁ and R_{b1} are CH₃, Rₐ₂ and R_{b2} are CH₃, Rₐ₃ and R_{b3} are

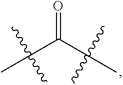 [SMILES: *C(*)=O]

and Rₐ₄ and R_{b4} are

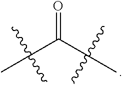 [SMILES: *C(*)=O]

In some variations of formula (11) described in the paragraphs above in which l₂ is 4, Rₐ₁ is H, R_{b1} is CH₃, Rₐ₂ and R_{b2} are CH₃, Rₐ₃ and R_{b3} are

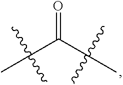 [SMILES: *C(*)=O]

and Rₐ₄ and R_{b4} are

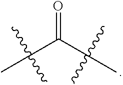 [SMILES: *C(*)=O]

In some variations of formula (11) described in the paragraphs above in which l₂ is 4, Rₐ₁ is H, R_{b1} is CH₃, Rₐ₂ is H, R_{b2} is CH₃, Rₐ₃ and R_{b3} are

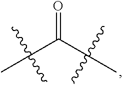 [SMILES: *C(*)=O]

and Rₐ₄ and R_{b4} are

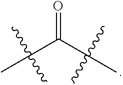 [SMILES: *C(*)=O]

In some variations of formula (11) described in the paragraphs above in which l₂ is 4, Rₐ₁ and R_{b1} are H, Rₐ₂ and R_{b2} are

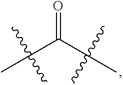 [SMILES: *C(*)=O]

Rₐ₃ and R_{b3} are

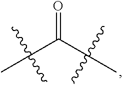 [SMILES: *C(*)=O]

and Rₐ₄ and R_{b4} are

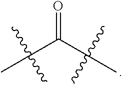 [SMILES: *C(*)=O]

In some variations of formula (11) described in the paragraphs above in which l₂ is 4, Rₐ₁ and R_{b1} are CH₃, Rₐ₂ and R_{b2} are

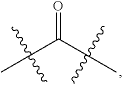 [SMILES: *C(*)=O]

Rₐ₃ and R_{b3} are

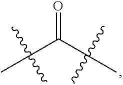 [SMILES: *C(*)=O]

and Rₐ₄ and R_{b4} are

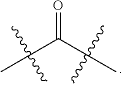 [SMILES: *C(*)=O]

In some variations of formula (11) described in the paragraphs above in which l₂ is 4, Rₐ₁ is H, R_{b1} is CH₃, Rₐ₂ and R_{b2} are

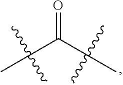 [SMILES: *C(*)=O]

Rₐ₃ and R_{b3} are

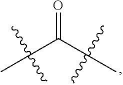 [SMILES: *C(*)=O]

and Rₐ₄ and R_{b4} are

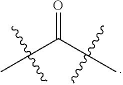 [SMILES: *C(*)=O]

In some variations of formula (11) described in the paragraphs above in which l₂ is 4, Rₐ₁ and R_{b1} are

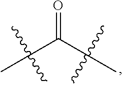 [SMILES: *C(*)=O]

Rₐ₂ and R_{b2} are

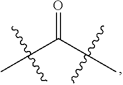 [SMILES: *C(*)=O]

Rₐ₃ and R_{b3} are

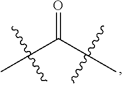 [SMILES: *C(*)=O]

and Rₐ₄ and R_{b4} are

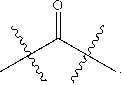 [SMILES: *C(*)=O]

In some variations of formula (11) described in the paragraphs above in which l₂ is 4, Rₐ₁ and R_{b1} are

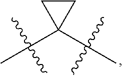 [SMILES: *C1(*)CC1]

Rₐ₂ and R_{b2} are H, Rₐ₃ and R_{b3} are H, and Rₐ₄ and R_{b4} are

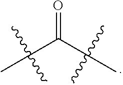 [SMILES: *C(*)=O]

In some variations of formula (11) described in the paragraphs above in which l₂ is 4, Rₐ₁ and R_{b1} are

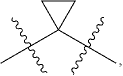 [SMILES: *C1(*)CC1]

Rₐ₂ and R_{b2} are CH₃, Rₐ₃ and R_{b3} are CH₃, and Rₐ₄ and R_{b4} are

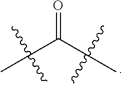 [SMILES: *C(*)=O]

In some variations of formula (11) described in the paragraphs above in which l₂ is 4, Rₐ₁ and R_{b1} are

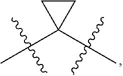 [SMILES: *C1(*)CC1]

Rₐ₂ and R_{b2} are H, Rₐ₃ and R_{b3} are CH₃, and Rₐ₄ and R_{b4} are

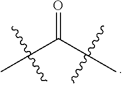 [SMILES: *C(*)=O]

In some variations of formula (11) described in the paragraphs above in which l₂ is 4, Rₐ₁ and R_{b1} are

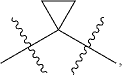 [SMILES: *C1(*)CC1]

Rₐ₂ and R_{b2} are

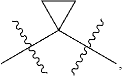 [SMILES: *C1(*)CC1]

Rₐ₃ and R_{b3} are H, and Rₐ₄ and R_{b4} are

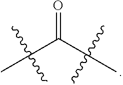 [SMILES: *C(*)=O]

In some variations of formula (11) described in the paragraphs above in which l₂ is 4, Rₐ₁ and R_{b1} are

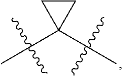 [SMILES: *C1(*)CC1]

Rₐ₂ and R_{b2} are

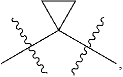 [SMILES: *C1(*)CC1]

Rₐ₃ and R_{b3} are CH₃, and Rₐ₄ and R_{b4} are

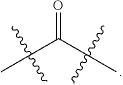 [SMILES: *C(*)=O]

In some variations of formula (11) described in the paragraphs above in which l₂ is 4, Rₐ₁ and R_{b1} are

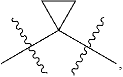 [SMILES: *C1(*)CC1]

Rₐ₂ and R_{b2} are

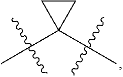 [SMILES: *C1(*)CC1]

Rₐ₃ and R_{b3} are CH₃, and Rₐ₄ and R_{b4} are

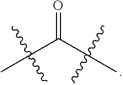 [SMILES: *C(*)=O]

In some variations of formula (11) described in the paragraphs above in which l₂ is 4, Rₐ₁ and R_{b1} are

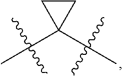 [SMILES: *C1(*)CC1]

Rₐ₂ and R_{b2} are

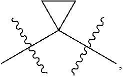 [SMILES: *C1(*)CC1]

Rₐ₃ and R_{b3} are H, and Rₐ₄ and R_{b4} are

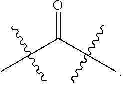 [SMILES: *C(*)=O]

In some variations of formula (11) described in the paragraphs above in which l₂ is 4, Rₐ₁ and R_{b1} are

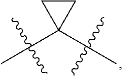 [SMILES: *C1(*)CC1]

Rₐ₂ and R_{b2} are

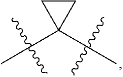 [SMILES: *C1(*)CC1]

Rₐ₃ and R_{b3} are

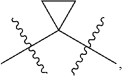 [SMILES: *C1(*)CC1]

and Rₐ₄ and R_{b4} are

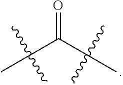 [SMILES: *C(*)=O]

In some variations of formula (11) described in the paragraphs above in which l₂ is 4, Rₐ₁ and R_{b1} are

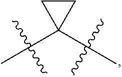 [SMILES: *C1(*)CC1]

Rₐ₂ and R_{b2} are

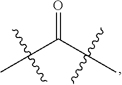 [SMILES: *C(*)=O]

Rₐ₃ and R_{b3} are H, and Rₐ₄ and R_{b4} are

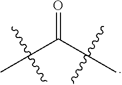 [SMILES: *C(*)=O]

In some variations of formula (11) described in the paragraphs above in which l₂ is 4, Rₐ₁ and R_{b1} are

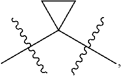 [SMILES: *C1(*)CC1]

Rₐ₂ and R_{b2} are

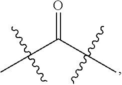 [SMILES: *C(*)=O]

Rₐ₃ and R_{b3} are CH₃, and Rₐ₄ and R_{b4} are

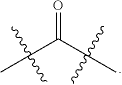 [SMILES: *C(*)=O]

In some variations of formula (11) described in the paragraphs above in which l₂ is 4, Rₐ₁ and R_{b1} are

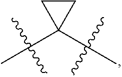 [SMILES: *C1(*)CC1]

Rₐ₂ and R_{b2} are

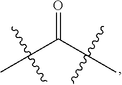 [SMILES: *C(*)=O]

Rₐ₃ and R_{b3} are CH₃, and Rₐ₄ and R_{b4} are

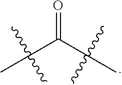 [SMILES: *C(*)=O]

In some variations of formula (11) described in the paragraphs above in which l₂ is 4, Rₐ₁ and R_{b1} are

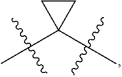 [SMILES: *C1(*)CC1]

Rₐ₂ and R_{b2} are

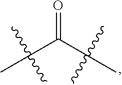 [SMILES: *C(*)=O]

Rₐ₃ and R_{b3} are H, and Rₐ₄ and R_{b4} are

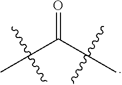 [SMILES: *C(*)=O]

In some variations of formula (11) described in the paragraphs above in which l₂ is 4, Rₐ₁ and R_{b1} are

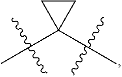 [SMILES: *C1(*)CC1]

Rₐ₂ and R_{b2} are

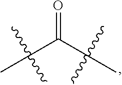 [SMILES: *C(*)=O]

Rₐ₃ and R_{b3} are

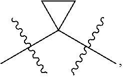 [SMILES: *C1(*)CC1]

and Rₐ₄ and R_{b4} are

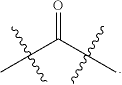 [SMILES: *C(*)=O]

In some variations of formula (11) described in the paragraphs above in which l₂ is 4, Rₐ₁ and R_{b1} are

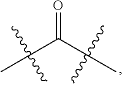 [SMILES: *C(*)=O]

Rₐ₂ and R_{b2} are

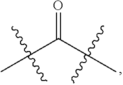 [SMILES: *C(*)=O]

Rₐ₃ and R_{b3} are H, and Rₐ₄ and R_{b4} are

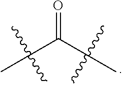 [SMILES: *C(*)=O]

In some variations of formula (11) described in the paragraphs above in which l₂ is 4, Rₐ₁ and R_{b1} are

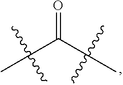 [SMILES: *C(*)=O]

Rₐ₂ and R_{b2} are

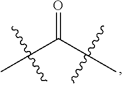 [SMILES: *C(*)=O]

Rₐ₃ and R_{b3} are CH₃, and Rₐ₄ and R_{b4} are

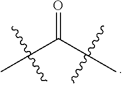 [SMILES: *C(*)=O]

In some variations of formula (11) described in the paragraphs above in which l₂ is 4, Rₐ₁ and R_{b1} are

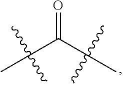 [SMILES: *C(*)=O]

Rₐ₂ and R_{b2} are

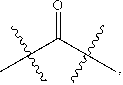 [SMILES: *C(*)=O]

Rₐ₃ and R_{b3} are CH₃, and Rₐ₄ and R_{b4} are

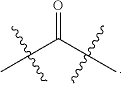 [SMILES: *C(*)=O]

In some variations of formula (11) described in the paragraphs above in which l₂ is 4, Rₐ₁ and R_{b1} are

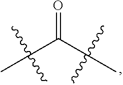 [SMILES: *C(*)=O]

Rₐ₂ and R_{b2} are

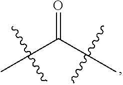 [SMILES: *C(*)=O]

Rₐ₃ and R_{b3} are H, and Rₐ₄ and R_{b4} are

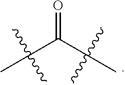 [SMILES: *C(*)=O]

In some variations of formula (11) described in the paragraphs above in which l₂ is 4, Rₐ₁ and R_{b1} are

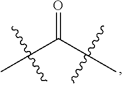 [SMILES: *C(*)=O]

Rₐ₂ and R_{b2} are

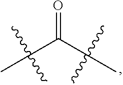 [SMILES: *C(*)=O]

Rₐ₃ and R_{b3} are

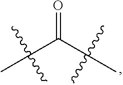 [SMILES: *C(*)=O]

and Rₐ₄ and R_{b4} are

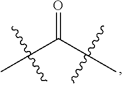 [SMILES: *C(*)=O]

In some variations of formula (11) described in the paragraphs above in which l₂ is 4, Rₐ₁ and R_{b1} are H, Rₐ₂ and R_{b2} are H, and Rₐ₃ and R_{b3} and Rₐ₄ and R_{b4} together are

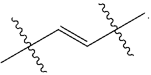 [SMILES: */C=C/*]

In some variations of formula (11) described in the paragraphs above in which l₂ is 4, Rₐ₁ and R_{b1} are H, Rₐ₂ and R_{b2} are CH₃, and Rₐ₃ and R_{b3} and Rₐ₄ and R_{b4} together are

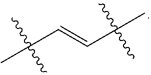 [SMILES: */C=C/*]

In some variations of formula (11) described in the paragraphs above in which l₂ is 4, Rₐ₁ and Rₐ₂ are H, R_{b1} and R_{b2} are CH₃, and Rₐ₃ and R_{b3} and Rₐ₄ and R_{b4} together are

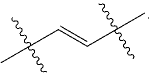 [SMILES: */C=C/*]

In some variations of formula (11) described in the paragraphs above in which l₂ is 4, Rₐ₁ and R_{b1} are H, Rₐ₂ and R_{b2} are

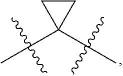 [SMILES: *C1(*)CC1]

and Rₐ₃ and R_{b3} and Rₐ₄ and R_{b4} together are

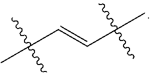 [SMILES: */C=C/*]

In some variations of formula (11) described in the paragraphs above in which l₂ is 4, Rₐ₁ and R_{b1} are

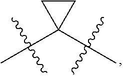 [SMILES: *C1(*)CC1]

Rₐ₂ and R_{b2} are CH₃, and Rₐ₃ and R_{b3} and Rₐ₄ and R_{b4} together are

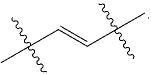 [SMILES: */C=C/*]

In some variations of formula (11) described in the paragraphs above in which l₂ is 4, Rₐ₁ is H, R_{b1} is CH₃, Rₐ₂ and R_{b2} are

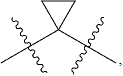 [SMILES: *C1(*)CC1]

and Rₐ₃ and R_{b3} and Rₐ₄ and R_{b4} together are

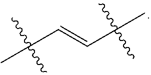 [SMILES: */C=C/*]

In some variations of formula (11) described in the paragraphs above in which l₂ is 4, Rₐ₁ and R_{b1} are H, Rₐ₂ and R_{b2} are

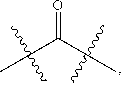 [SMILES: *C(*)=O]

and Rₐ₃ and R_{b3} and Rₐ₄ and R_{b4} together are

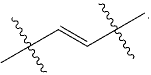 [SMILES: */C=C/*]

In some variations of formula (11) described in the paragraphs above in which l₂ is 4, Rₐ₁ and R_{b1} are

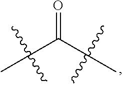 [SMILES: *C(*)=O]

Rₐ₂ and R_{b2} are CH₃, and Rₐ₃ and R_{b3} and Rₐ₄ and R_{b4} together are

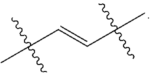 [SMILES: */C=C/*]

In some variations of formula (11) described in the paragraphs above in which l₂ is 4, Rₐ₁ is H, R_{b1} is CH₃, Rₐ₂ and R_{b2} are

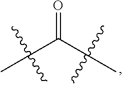 [SMILES: *C(*)=O]

and Rₐ₃ and R_{b3} and Rₐ₄ and R_{b4} together are

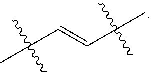 [SMILES: */C=C/*]

In some variations of formula (11) described in the paragraphs above in which l₂ is 4, Rₐ₁ and R_{b1} are

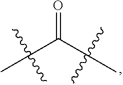 [SMILES: *C(*)=O]

Rₐ₂ and R_{b2} are

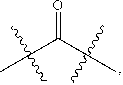 [SMILES: *C(*)=O]

and Rₐ₃ and R_{b3} and Rₐ₄ and R_{b4} together are

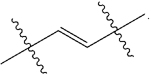 [SMILES: */C=C/*]

In some variations of formula (11) described in the paragraphs above in which l₂ is 4, Rₐ₁ and R_{b1} are

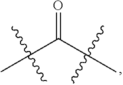 [SMILES: *C(*)=O]

Rₐ₂ and R_{b2} are

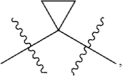 [SMILES: *C1(*)CC1]

and Rₐ₃ and R_{b3} and Rₐ₄ and R_{b4} together are

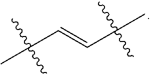 [SMILES: */C=C/*]

In some variations of formula (11) described in the paragraphs above in which l₂ is 4, Rₐ₁ and R_{b1} are

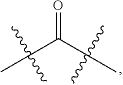 [SMILES: *C(*)=O]

Rₐ₂ and R_{b2} are

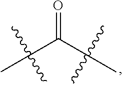 [SMILES: *C(*)=O]

and Rₐ₃ and R_{b3} and Rₐ₄ and R_{b4} together are

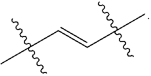 [SMILES: */C=C/*]

In some variations of formula (11) described in the paragraphs above in which l₂ is 4, Rₐ₁ and R_{b1} are

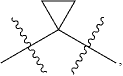 [SMILES: *C1(*)CC1]

Rₐ₂ and R_{b2} are

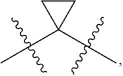 [SMILES: *C1(*)CC1]

and Rₐ₃ and R_{b3} and Rₐ₄ and R_{b4} together are

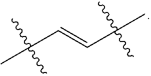 [SMILES: */C=C/*]

In some variations of formula (11) described in the paragraphs above in which l₂ is 4, Rₐ₁ and R_{b1} and Rₐ₂ and R_{b2} together are

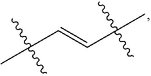 [SMILES: */C=C/*]

and Rₐ₃ and R_{b3} and Rₐ₄ and R_{b4} together are

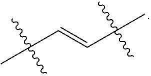 [SMILES: */C=C/*]

In some variations of formula (11) described in the paragraphs above ring A is

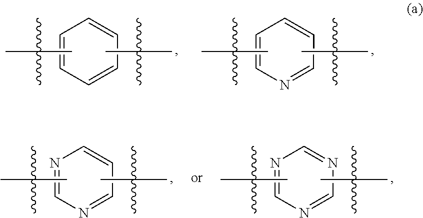

optionally substituted with halo or C1-C6 linear or branched alkyl;

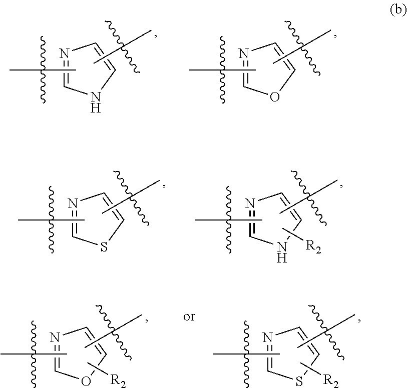

optionally substituted with halo or C1-C6 linear or branched alkyl; or

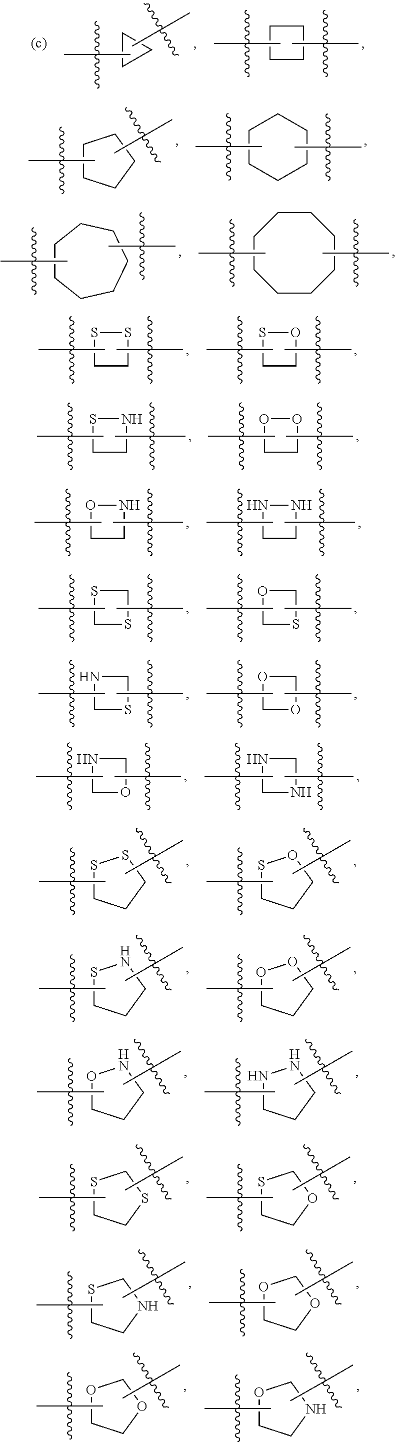

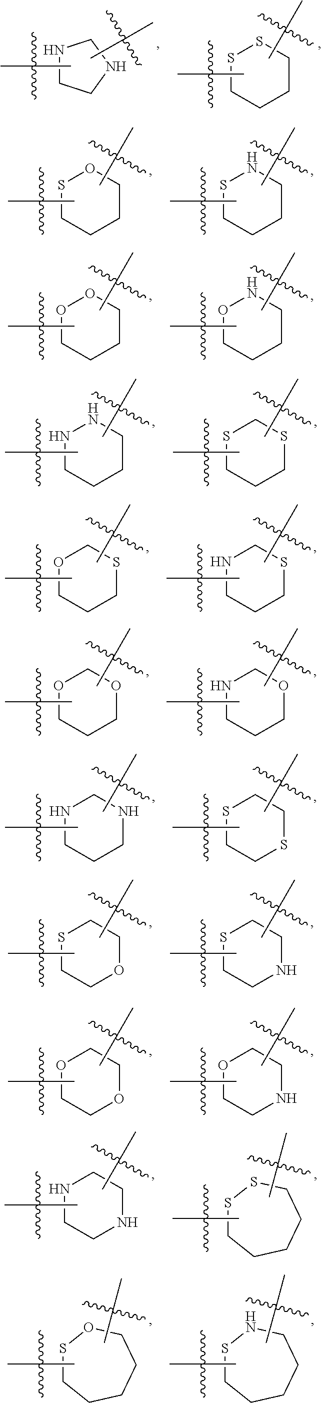

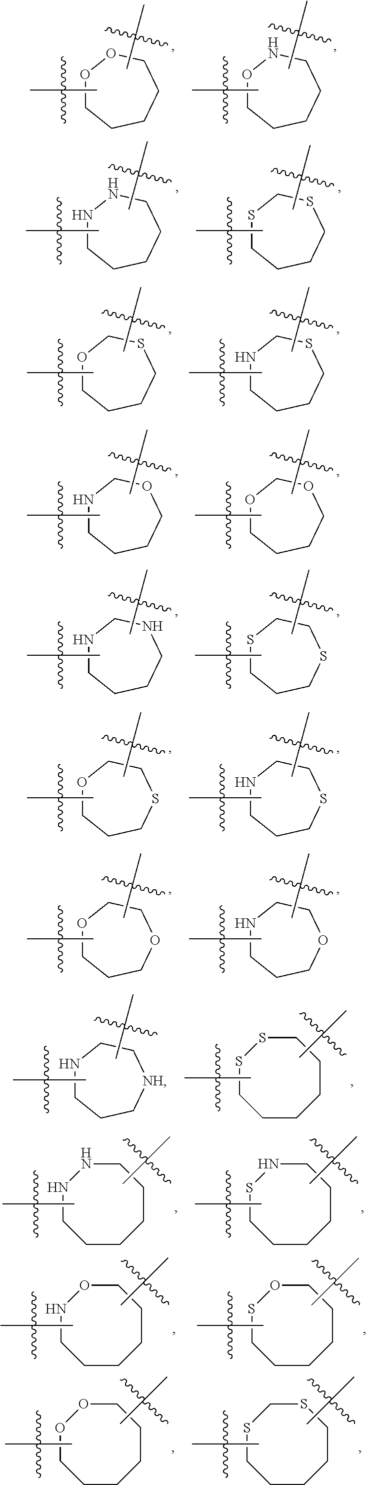

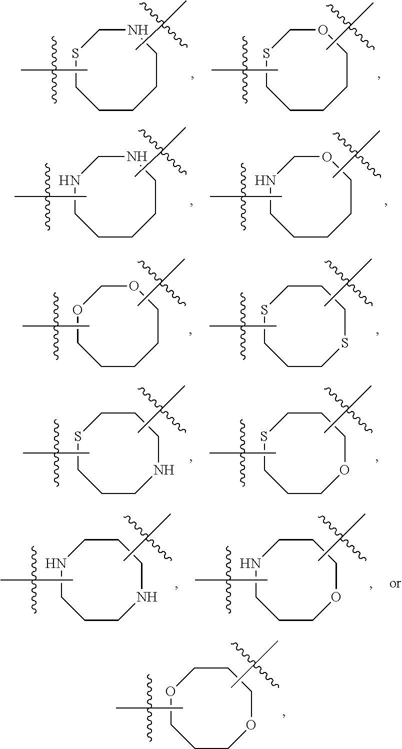

optionally substituted with halo or C1-C6 linear or branched alkyl.

In some variations of formula (11) described in the paragraphs above, n is 1-4, 1-3, 1-2, 2-4, 2-3, 3-4, 1, 2, 3, or 4.

In some variations of formula (11) described in the paragraphs above, ring A is substituted with halo. In some variations, the halo is F, Br, I, or Cl.

In some variations of formula (11) described in the paragraphs above, ring A is substituted with C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (11) described in the paragraphs above, D1 is safranin-O. In some variations of formula (11) described in the paragraphs above, D2 is safranin-O. In some variations of formula (11) described in the paragraphs above, D1 and D2 are safranin-O.

In some variations of formula (11) described in the paragraph above, the pendant phenyl ring of D1 is unsubstituted. In some variations of formula (11) described in the paragraph above, the pendant phenyl ring of D1 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D1 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D1 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (11) described in the paragraph above, the pendant phenyl ring of D2 is unsubstituted. In some variations of formula (11) described in the paragraph above, the pendant phenyl ring of D2 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D2 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D2 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some of the variations of formula (11) described above, D1 and D2 are safranin-O moieties, as shown in formula (11a):

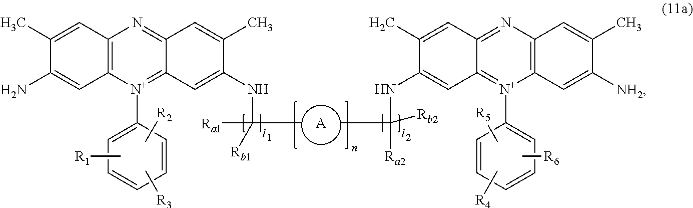

in which l₁, l₂, n, ring A, Rₐ₁, R_{b1} Rₐ₂, and R_{b2} are as described in the paragraphs above, R₁, R₂, R₃, R₄, R₅, and R₆ independently are absent or independently are selected from —NH₂, —NHR, —NR₂, —OH, —O⁻, —NHCOCH₃, —NHCOR, —OCH₃, —OR, —C₂H₅, —R, —C₆H₅, —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR), and R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

One of skill in the art can readily visualize and prepare other cationic multimers falling within formula (11) in which other cationic dye moieties are used in place of one or both of the safranin-O moieties.

Some cationic dye dimers fall within formula (12):

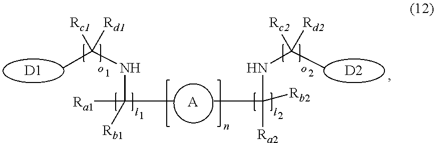

in which each of D1 and D2 is a cationic dye moiety; l₁, l₂, n, o₁, and o₂ independently are 1-4; ring A is aryl, heteroaryl, cycloalkyl, or heterocyclyl; for each independent instance of Rₐ₁ and R_{b1}, Rₐ₁ and R_{b1} (1) independently are H or CH₃, or (2) Rₐ₁ and R_{b1} are

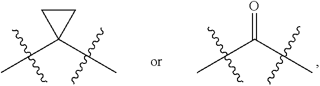

or (3) two of CRₐ₁R_{b1} are

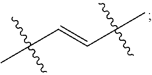 [SMILES: */C=C/*]

for each independent instance of Rₐ₂ and R_{b2}, Rₐ₂ and R_{b2} (1) independently are H or CH₃, or (2) Rₐ₂ and R_{b2} are

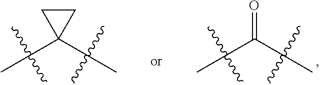

or (3) two of CRₐ₂R_{b2} are

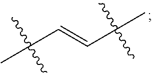 [SMILES: */C=C/*]

for each independent instance of R_{c1} and R_{d1}, R_{c1} and R_{d1} (1) independently are H or CH₃, or (2) R_{c1} and R_{d1} are

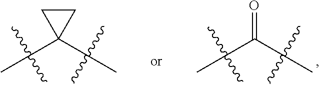

or (3) two of CR_{c1}R_{d1} are

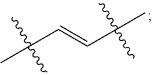 [SMILES: */C=C/*]

for each independent instance of R_{c2} and R_{d2}, R_{c1} and R_{d2} (1) independently are H or CH₃, or (2) R_{c2} and R_{d2} are

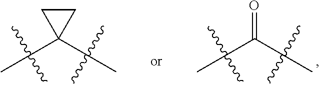

or (3) two of CR_{c2}R_{d2} are

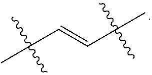 [SMILES: */C=C/*]

In some variations of formula (12), the cationic dye moiety is selected from the group consisting of safranin-O, toluidine blue, azure A, azure B, azure C, acridine orange, acriflavine, and methylene blue. In some variations of formula (12), D1 and D2 are different cationic dye moieties. In other variations of formula (12), D1 and D2 are the same cationic dye moiety.

In some variations of formula (12) described in the paragraphs above, l₁ is 1-4, 1-3, 1-2, 2-4, 2-3, 3-4, 1, 2, 3, or 4.

In some variations of formula (12) described in the paragraphs above, l₂ is 1-4, 1-3, 1-2, 2-4, 2-3, 3-4, 1, 2, 3, or 4.

In some previous variations of formula (12) described in the paragraphs above, o₁ is 1-4, 1-3, 1-2, 2-4, 2-3, 3-4, 1, 2, 3, or 4.

In some previous variations of formula (12) described in the paragraphs above, o₂ is 1-4, 1-3, 1-2, 2-4, 2-3, 3-4, 1, 2, 3, or 4.

In some variations of formula (12) described in the paragraphs above in which l₁ is 1, Rₐ₁ and R_{b1} are both H. In some variations of formula (12) described in the paragraphs above in which l₁ is 1, Rₐ₁ is H and R_{b1} is CH₃. In some variations of formula (12) described in the paragraphs above in which l₁ is 1, Rₐ₁ and R_{b1} are both CH₃. In some variations of formula (12) described in the paragraphs above in which l₁ is 1, Rₐ₁ and R_{b1} are

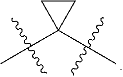 [SMILES: *C1(*)CC1]

In some variations of formula (12) described in the paragraphs above in which l₁ is 1, Rₐ₁ and R_{b1} are

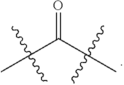 [SMILES: *C(*)=O]

In variations of formula (12) described in the paragraphs above in which l₁ is 2, the two instances of Rₐ and R_{b} are indicated as Rₐ₁ and R_{b1} and Rₐ₂ and R_{b2}, respectively. In some variations of formula (12) described in the paragraphs above in which l₁ is 2, each of Rₐ₁ and R_{b1} and Rₐ₂ and R_{b2} is H. In some variations of formula (12) described in the paragraphs above in which l₁ is 2, each of Rₐ₁ and R_{b1} and Rₐ₂ and R_{b2} is CH₃. In some variations of formula (12) described in the paragraphs above in which l₁ is 2, each of Rₐ₁ and Rₐ₂ is H and each of R_{b1} and R_{b2} is CH₃. In some variations of formula (12) described in the paragraphs above in which l₁ is 2, each of Rₐ₁, Rₐ₂, and R_{b1} is H and R_{b2} is CH₃.

In some variations of formula (12) described in the paragraphs above in which l₁ is 2, each of Rₐ₁ and R_{b1} is H and Rₐ₂ and R_{b2} are

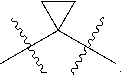 [SMILES: *C1(*)CC1]

In some variations of formula (12) described in the paragraphs above in which l₁ is 2, each of Rₐ₁ and R_{b1} is H and Rₐ₂ and R_{b2} are

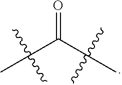 [SMILES: *C(*)=O]

In some variations of formula (12) described in the paragraphs above in which l₁ is 2, each of Rₐ₁ and R_{b1} is CH₃ and Rₐ₂ and R_{b2} are

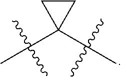 [SMILES: *C1(*)CC1]

In some variations of formula (12) described in the paragraphs above in which l₁ is 2, each of Rₐ₁ and R_{b1} is CH₃ and Rₐ₂ and R_{b2} are

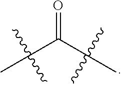 [SMILES: *C(*)=O]

In some variations of formula (12) described in the paragraphs above in which l₁ is 2, Rₐ₁ is H, R_{b1} is CH₃, and Rₐ₂ and R_{b2} are

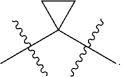 [SMILES: *C1(*)CC1]

In some variations of formula (12) described in the paragraphs above in which l₁ is 2, Rₐ₁ is H, R_{b1} is CH₃, and Rₐ₂ and R_{b2} are

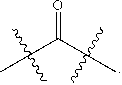 [SMILES: *C(*)=O]

In some variations of formula (12) described in the paragraphs above in which l₁ is 2, Rₐ₁ and R_{b1} and Rₐ₂ and R_{b2} together are

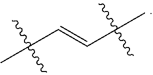 [SMILES: */C=C/*]

In some variations of formula (12) described in the paragraphs above in which l₁ is 2, Rₐ₁ and R_{b1} are

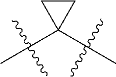 [SMILES: *C1(*)CC1]

Rₐ₂ and R_{b2} are

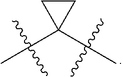 [SMILES: *C1(*)CC1]

In some variations of formula (12) described in the paragraphs above in which l₁ is 2, Rₐ₁ and R_{b1} are

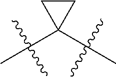 [SMILES: *C1(*)CC1]

Rₐ₂ and R_{b2} are

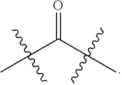 [SMILES: *C(*)=O]

In some variations of formula (12) described in the paragraphs above in which l₁ is 2, Rₐ₁ and R_{b1} are

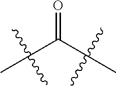 [SMILES: *C(*)=O]

Rₐ₂ and R_{b2} are

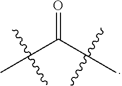 [SMILES: *C(*)=O]

In variations of formula (12) described in the paragraphs above in which l₁ is 3, the three instances of Rₐ and R_{b} are indicated as Rₐ₁ and R_{b1}; Rₐ₂ and R_{b2}; and Rₐ₃ and R_{b3}, respectively. In some variations of formula (12) described in the paragraphs above in which l₁ is 3, In some variations of formula (12) described in the paragraphs above in which l₁ is 3, each of Rₐ₁, R_{b1}, Rₐ₂, R_{b2}, Rₐ₃, and R_{b3} is H. In some variations of formula (12) described in the paragraphs above in which l₁ is 3, each of Rₐ₁, R_{b1}, Rₐ₂, R_{b2}, Rₐ₃, and R_{b3} is CH₃. In some variations of formula (12) described in the paragraphs above in which l₁ is 3, each of Rₐ₁, R_{b1}, Rₐ₂, and R_{b2}, is H and each of Rₐ₃ and R_{b3} is CH₃. In some variations of formula (12) described in the paragraphs above in which l₁ is 3, each of Rₐ₁, R_{b1}, Rₐ₂, and R_{b2}, is CH₃ and each of Rₐ₃ and R_{b3} is H. In some variations of formula (12) described in the paragraphs above in which l₁ is 3, Rₐ₁ is H and each of Rₐ₂, Rₐ₃, R_{b1}, R_{b2}, and R_{b3} is CH₃. In some variations of formula (12) described in the paragraphs above in which l₁ is 3, each of Rₐ₁ and Rₐ₂ is H and each of Rₐ₃, R_{b1}, R_{b2}, an R_{b3} is CH₃. In some variations of formula (12) described in the paragraphs above in which l₁ is 3, each of Rₐ₁, Rₐ₂, and Rₐ₃ is H and each of R_{b1}, R_{b2}, an R_{b3} is CH₃.

In some variations of formula (12) described in the paragraphs above in which l₁ is 3, each of Rₐ₁, R_{b1}, Rₐ₂, and R_{b2}, is H and Rₐ₃ and R_{b3} are

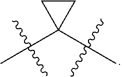 [SMILES: *C1(*)CC1]

In some variations of formula (12) described in the paragraphs above in which l₁ is 3, each of Rₐ₁, R_{b1}, Rₐ₂, and R_{b2}, is CH₃ and Rₐ₃ and R_{b3} are

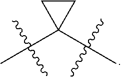 [SMILES: *C1(*)CC1]

In some variations of formula (12) described in the paragraphs above in which l₁ is 3, Rₐ₁ is H and each of Rₐ₂, R_{b1}, and R_{b2} is CH₃, and Rₐ₃ and R_{b3} are

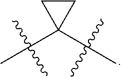 [SMILES: *C1(*)CC1]

In some variations of formula (12) described in the paragraphs above in which l₁ is 3, each of Rₐ₁ and Rₐ₂ is H and each of R_{b1} and R_{b2} is CH₃, and Rₐ₃ and R_{b3} are

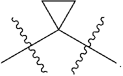 [SMILES: *C1(*)CC1]

In some variations of formula (12) described in the paragraphs above in which l₁ is 3, each of Rₐ₁, R_{b1}, Rₐ₂, and R_{b2}, is H and Rₐ₃ and R_{b3} are

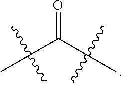 [SMILES: *C(*)=O]

In some variations of formula (12) described in the paragraphs above in which l₁ is 3, each of Rₐ₁, R_{b1}, Rₐ₂, and R_{b2}, is CH₃ and Rₐ₃ and R_{b3} are

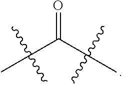 [SMILES: *C(*)=O]

In some variations of formula (12) described in the paragraphs above in which l₁ is 3, Rₐ₁ is H and each of Rₐ₂, R_{b1}, and R_{b2} is CH₃, and Rₐ₃ and R_{b3} are

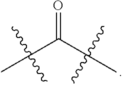 [SMILES: *C(*)=O]

In some variations of formula (12) described in the paragraphs above in which l₁ is 3, each of Rₐ₁ and Rₐ₂ is H and each of R_{b1} and R_{b2} is CH₃, and Rₐ₃ and R_{b3} are

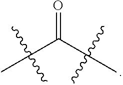 [SMILES: *C(*)=O]

In some variations of formula (12) described in the paragraphs above in which l₁ is 3, Rₐ₁ and R_{b1} are

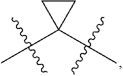 [SMILES: *C1(*)CC1]

Rₐ₂ and R_{b2} are

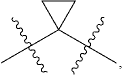 [SMILES: *C1(*)CC1]

and each of Rₐ₃ and R_{b3} is H. In some variations of formula (12) described in the paragraphs above in which l₁ is 3, Rₐ₁ and R_{b1} are

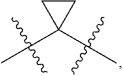 [SMILES: *C1(*)CC1]

Rₐ₂ and R_{b2} are

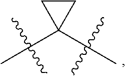 [SMILES: *C1(*)CC1]

and each of Rₐ₃ and R_{b3} is CH₃. In some variations of formula (12) described in the paragraphs above in which l₁ is 3, Rₐ₁ and R_{b1} are

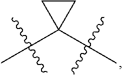 [SMILES: *C1(*)CC1]

Rₐ₂ and R_{b2} are

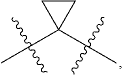 [SMILES: *C1(*)CC1]

Rₐ₃ is H, and R_{b3} is CH₃. In some variations of formula (12) described in the paragraphs above in which l₁ is 3, Rₐ₁ and R_{b1} are

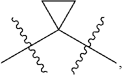 [SMILES: *C1(*)CC1]

Rₐ₂ and R_{b2} are

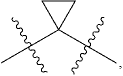 [SMILES: *C1(*)CC1]

and each of Rₐ₃ and R_{b3} is H. In some variations of formula (12) described in the paragraphs above in which l₁ is 3, Rₐ₁ and R_{b1} are

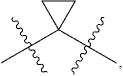 [SMILES: *C1(*)CC1]

Rₐ₂ and R_{b2} are

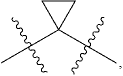 [SMILES: *C1(*)CC1]

and each of Rₐ₃ and R_{b3} is CH₃. In some variations of formula (12) described in the paragraphs above in which l₁ is 3, Rₐ₁ and R_{b1} are

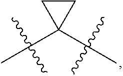 [SMILES: *C1(*)CC1]

Rₐ₂ and R_{b2} are

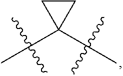 [SMILES: *C1(*)CC1]

Rₐ₃ is H, and R_{b3} is CH₃.

In some variations of formula (12) described in the paragraphs above in which l₁ is 3, Rₐ₁ and R_{b1} are

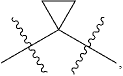 [SMILES: *C1(*)CC1]

Rₐ₂ and R_{b2} are

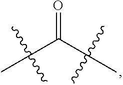 [SMILES: *C(*)=O]

and each of Rₐ₃ and R_{b3} is H. In some variations of formula (12) described in the paragraphs above in which l₁ is 3, Rₐ₁ and R_{b1} are

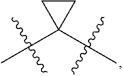 [SMILES: *C1(*)CC1]

Rₐ₂ and R_{b2} are

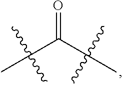 [SMILES: *C(*)=O]

and each of Rₐ₃ and R_{b3} is CH₃. In some variations of formula (12) described in the paragraphs above in which l₁ is 3, Rₐ₁ and R_{b1} are

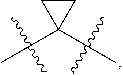 [SMILES: *C1(*)CC1]

Rₐ₂ and R_{b2} are

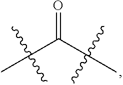 [SMILES: *C(*)=O]

Rₐ₃ is H, and R_{b3} is CH₃. In some variations of formula (12) described in the paragraphs above in which l₁ is 3, Rₐ₁ and R_{b1} are

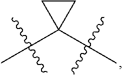 [SMILES: *C1(*)CC1]

Rₐ₂ and R_{b2} are

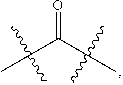 [SMILES: *C(*)=O]

and each of Rₐ₃ and R_{b3} is H. In some variations of formula (12) described in the paragraphs above in which l₁ is 3, Rₐ₁ and R_{b1} are

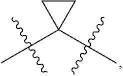 [SMILES: *C1(*)CC1]

Rₐ₂ and R_{b2} are

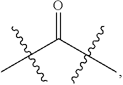 [SMILES: *C(*)=O]

and each of Rₐ₃ and R_{b3} is CH₃. In some variations of formula (12) described in the paragraphs above in which l₁ is 3, Rₐ₁ and R_{b1} are

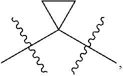 [SMILES: *C1(*)CC1]

Rₐ₂ and R_{b2} are

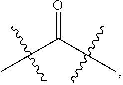 [SMILES: *C(*)=O]

Rₐ₃ is H, and R_{b3} is CH₃.

In some variations of formula (12) described in the paragraphs above in which l₁ is 3, Rₐ₁ and R_{b1} are

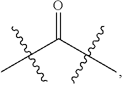 [SMILES: *C(*)=O]

Rₐ₂ and R_{b2} are

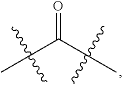 [SMILES: *C(*)=O]

and each of Rₐ₃ and R_{b3} is H. In some variations of formula (12) described in the paragraphs above in which l₁ is 3, Rₐ₁ and R_{b1} are

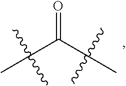 [SMILES: *C(*)=O]

Rₐ₂ and R_{b2} are

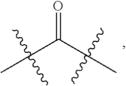 [SMILES: *C(*)=O]

and each of Rₐ₃ and R_{b3} is CH₃. In some variations of formula (12) described in the paragraphs above in which l₁ is 3, Rₐ₁ and R_{b1} are

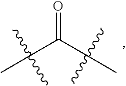 [SMILES: *C(*)=O]

Rₐ₂ and R_{b2} are

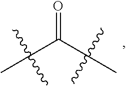 [SMILES: *C(*)=O]

Rₐ₃ is H, and R_{b3} is CH₃. In some variations of formula (12) described in the paragraphs above in which l₁ is 3, Rₐ₁ and R_{b1} are

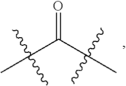 [SMILES: *C(*)=O]

Rₐ₂ and R_{b2} are

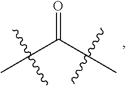 [SMILES: *C(*)=O]

and each of Rₐ₃ and R_{b3} is H. In some variations of formula (12) described in the paragraphs above in which l₁ is 3, Rₐ₁ and R_{b1} are

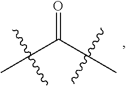 [SMILES: *C(*)=O]

Rₐ₂ and R_{b2} are

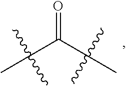 [SMILES: *C(*)=O]

and each of Rₐ₃ and R_{b3} is CH₃. In some variations of formula (12) described in the paragraphs above in which l₁ is 3, Rₐ₁ and R_{b1} are

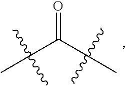 [SMILES: *C(*)=O]

Rₐ₂ and R_{b2} are

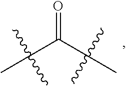 [SMILES: *C(*)=O]

Rₐ₃ is H, and R_{b3} is CH₃.

In some variations of formula (12) described in the paragraphs above in which l₁ is 3, Rₐ₁ and R_{b1} and Rₐ₂ and R_{b2} together are

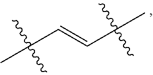 [SMILES: */C=C/*]

and each of Rₐ₃ and R_{b3} is H. In some variations of formula (12) described in the paragraphs above in which l₁ is 3, Rₐ₁ and R_{b1} and Rₐ₂ and R_{b2} together are

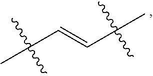 [SMILES: */C=C/*]

and each of Rₐ₃ and R_{b3} is CH₃. In some variations of formula (12) described in the paragraphs above in which l₁ is 3, Rₐ₁ and R_{b1} and Rₐ₂ and R_{b2} together are

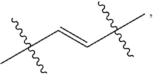 [SMILES: */C=C/*]

Rₐ₃ is H, and R_{b3} is CH₃. In some variations of formula (12) described in the paragraphs above in which l₁ is 3, Rₐ₁ and R_{b1} and Rₐ₂ and R_{b2} together are

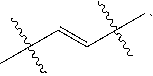 [SMILES: */C=C/*]

and Rₐ₃ and R_{b3} are

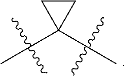 [SMILES: *C1(*)CC1]

In some variations of formula (12) described in the paragraphs above in which l₁ is 3, Rₐ₁ and R_{b1} and Rₐ₂ and R_{b2} together are

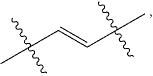 [SMILES: */C=C/*]

and Rₐ₃ and R_{b3} are

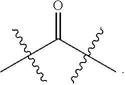 [SMILES: *C(*)=O]

In variations of formula (12) described in the paragraphs above in which l₁ is 4, the three instances of Rₐ and R_{b} are indicated as Rₐ₁ and R_{b1}; Rₐ₂ and R_{b2}; Rₐ₃ and R_{b3}; and Rₐ₄ and R_{b4}, respectively. In some variations of formula (12) described in the paragraphs above in which l₁ is 4, In some variations of formula (12) described in the paragraphs above in which l₁ is 4, each of Rₐ₁, R_{b1}, Rₐ₂, R_{b2}, Rₐ₃, R_{b3}, Rₐ₄, and R_{b4} is H. In some variations of formula (12) described in the paragraphs above in which l₁ is 4, each of Rₐ₁, R_{b1}, Rₐ₂, R_{b2}, Rₐ₃, R_{b3}, Rₐ₄, and R_{b4} is CH₃. In some variations of formula (12) described in the paragraphs above in which l₁ is 4, each of Rₐ₁, R_{b1}, Rₐ₂, R_{b2}, Rₐ₃, and R_{b3} is H and each of Rₐ₄ and R_{b4} is CH₃. In some variations of formula (12) described in the paragraphs above in which l₁ is 4, each of Rₐ₁, R_{b1}, Rₐ₂, and R_{b2} is H and each of Rₐ₃, R_{b3}, Rₐ₄, and R_{b4} is CH₃. In some variations of formula (12) described in the paragraphs above in which l₁ is 4, each of Rₐ₁, R_{b1}, Rₐ₂, R_{b2}, Rₐ₃, and R_{b3} is CH₃ and each of Rₐ₄ and R_{b4} is H.

In some variations of formula (12) described in the paragraphs above in which l₁ is 4, each of Rₐ₁, Rₐ₂, and Rₐ₃ is H and each of R_{b1}, R_{b2}, and R_{b3} is CH₃. In some variations of formula (12) described in the paragraphs above in which l₁ is 4, Rₐ₁ is H, R_{b1} is CH₃, and each of Rₐ₂, R_{b2}, Rₐ₃, and R_{b3} is CH₃. In some variations of formula (12) described in the paragraphs above in which l₁ is 4, Rₐ₁ is H, R_{b1} is CH₃, and each of Rₐ₂, R_{b2}, Rₐ₃, and R_{b3} is H. In some variations of formula (12) described in the paragraphs above in which l₁ is 4, each of Rₐ₁ and Rₐ₂ is H, each of R_{b1} and R_{b2} is CH₃, and each of Rₐ₃ and R_{b3} is H. In some variations of formula (12) described in the paragraphs above in which l₁ is 4, each of Rₐ₁ and Rₐ₂ is H, each of R_{b1} and R_{b2} is CH₃, and each of Rₐ₃ and R_{b3} is CH₃.

In some variations of formula (12) described in the paragraphs above in which l₁ is 4, Rₐ₁ and R_{b1} are H, Rₐ₂ and R_{b2} are H, Rₐ₃ and R_{b3} are H, and Rₐ₄ and R_{b4} are

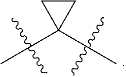 [SMILES: *C1(*)CC1]

In some variations of formula (12) described in the paragraphs above in which l₁ is 4, Rₐ₁ and R_{b1} are H, Rₐ₂ and R_{b2} are H, Rₐ₃ and R_{b3} are CH₃, and Rₐ₄ and R_{b4} are

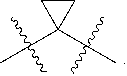 [SMILES: *C1(*)CC1]

In some variations of formula (12) described in the paragraphs above in which l₁ is 4, Rₐ₁ and R_{b1} are H, Rₐ₂ and R_{b2} are CH₃, Rₐ₃ and R_{b3} are CH₃, and Rₐ₄ and R_{b4} are

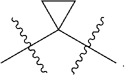 [SMILES: *C1(*)CC1]

In some variations of formula (12) described in the paragraphs above in which l₁ is 4, Rₐ₁ and R_{b1} are CH₃, Rₐ₂ and R_{b2} are CH₃, Rₐ₃ and R_{b3} are CH₃, and Rₐ₄ and R_{b4} are

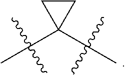 [SMILES: *C1(*)CC1]

In some variations of formula (12) described in the paragraphs above in which l₁ is 4, Rₐ₁ and R_{b1} are H, Rₐ₂ and R_{b2} are H, Rₐ₃ and R_{b3} are

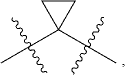 [SMILES: *C1(*)CC1]

and Rₐ₄ and R_{b4} are

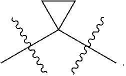 [SMILES: *C1(*)CC1]

In some variations of formula (12) described in the paragraphs above in which l₁ is 4, Rₐ₁ and R_{b1} are H, Rₐ₂ and R_{b2} are CH₃, Rₐ₃ and R_{b3} are

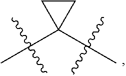 [SMILES: *C1(*)CC1]

and Rₐ₄ and R_{b4} are

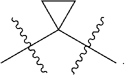 [SMILES: *C1(*)CC1]

In some variations of formula (12) described in the paragraphs above in which l₁ is 4, Rₐ₁ and R_{b1} are CH₃, Rₐ₂ and R_{b2} are CH₃, Rₐ₃ and R_{b3} are

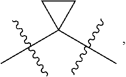 [SMILES: *C1(*)CC1]

and Rₐ₄ and R_{b4} are

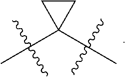 [SMILES: *C1(*)CC1]

In some variations of formula (12) described in the paragraphs above in which l₁ is 4, Rₐ₁ is H, R_{b1} is CH₃, Rₐ₂ and R_{b2} are CH₃, Rₐ₃ and R_{b3} are

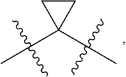 [SMILES: *C1(*)CC1]

and Rₐ₄ and R_{b4} are

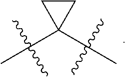 [SMILES: *C1(*)CC1]

In some variations of formula (12) described in the paragraphs above in which l₁ is 4, Rₐ₁ is H, R_{b1} is CH₃, Rₐ₂ is H, R_{b2} is CH₃, Rₐ₃ and R_{b3} are

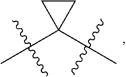 [SMILES: *C1(*)CC1]

and Rₐ₄ and R_{b4} are

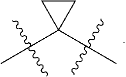 [SMILES: *C1(*)CC1]

In some variations of formula (12) described in the paragraphs above in which l₁ is 4, Rₐ₁ and R_{b1} are H, Rₐ₂ and R_{b2} are

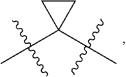 [SMILES: *C1(*)CC1]

Rₐ₃ and R_{b3} are

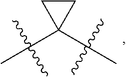 [SMILES: *C1(*)CC1]

and Rₐ₄ and R_{b4} are

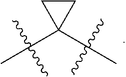 [SMILES: *C1(*)CC1]

In some variations of formula (12) described in the paragraphs above in which l₁ is 4, Rₐ₁ and R_{b1} are CH₃, Rₐ₂ and R_{b2} are

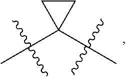 [SMILES: *C1(*)CC1]

Rₐ₃ and R_{b3} are

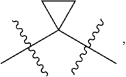 [SMILES: *C1(*)CC1]

and Rₐ₄ and R_{b4} are

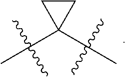 [SMILES: *C1(*)CC1]

In some variations of formula (12) described in the paragraphs above in which l₁ is 4, Rₐ₁ is H, R_{b1} is CH₃, Rₐ₂ and R_{b2} are

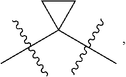 [SMILES: *C1(*)CC1]

Rₐ₃ and R_{b3} are

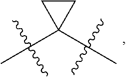 [SMILES: *C1(*)CC1]

and Rₐ₄ and R_{b4} are

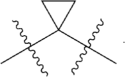 [SMILES: *C1(*)CC1]

In some variations of formula (12) described in the paragraphs above in which l₁ is 4, Rₐ₁ and R_{b1} are

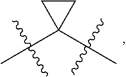 [SMILES: *C1(*)CC1]

Rₐ₂ and R_{b2} are

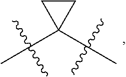 [SMILES: *C1(*)CC1]

Rₐ₃ and R_{b3} are

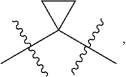 [SMILES: *C1(*)CC1]

and Rₐ₄ and R_{b4} are

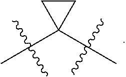 [SMILES: *C1(*)CC1]

In some variations of formula (12) described in the paragraphs above in which l₁ is 4, Rₐ₁ and R_{b1} are H, Rₐ₂ and R_{b2} are H, Rₐ₃ and R_{b3} are H, and Rₐ₄ and R_{b4} are

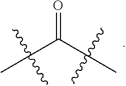 [SMILES: *C(*)=O]

In some variations of formula (12) described in the paragraphs above in which l₁ is 4, Rₐ₁ and R_{b1} are H, Rₐ₂ and R_{b2} are H, Rₐ₃ and R_{b3} are CH₃, and Rₐ₄ and R_{b4} are

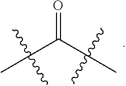 [SMILES: *C(*)=O]

In some variations of formula (12) described in the paragraphs above in which l₁ is 4, Rₐ₁ and R_{b1} are H, Rₐ₂ and R_{b2} are CH₃, Rₐ₃ and R_{b3} are CH₃, and Rₐ₄ and R_{b4} are

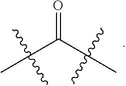 [SMILES: *C(*)=O]

In some variations of formula (12) described in the paragraphs above in which l₁ is 4, Rₐ₁ and R_{b1} are CH₃, Rₐ₂ and R_{b2} are CH₃, Rₐ₃ and R_{b3} are CH₃, and Rₐ₄ and R_{b4} are

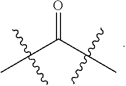 [SMILES: *C(*)=O]

In some variations of formula (12) described in the paragraphs above in which l₁ is 4, Rₐ₁ and R_{b1} are H, Rₐ₂ and R_{b2} are H, Rₐ₃ and R_{b3} are

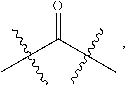 [SMILES: *C(*)=O]

and Rₐ₄ and R_{b4} are v. In some variations of formula (12) described in the paragraphs above in which l₁ is 4, Rₐ₁ and R_{b1} are H, Rₐ₂ and R_{b2} are CH₃, Rₐ₃ and R_{b3} are

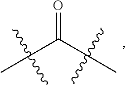 [SMILES: *C(*)=O]

and Rₐ₄ and R_{b4} are

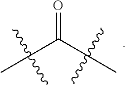 [SMILES: *C(*)=O]

In some variations of formula (12) described in the paragraphs above in which l₁ is 4, Rₐ₁ and R_{b1} are CH₃, Rₐ₂ and R_{b2} are CH₃, Rₐ₃ and R_{b3} are

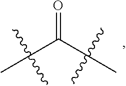 [SMILES: *C(*)=O]

and Rₐ₄ and R_{b4} are

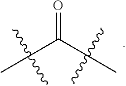 [SMILES: *C(*)=O]

In some variations of formula (12) described in the paragraphs above in which l₁ is 4, Rₐ₁ is H, R_{b1} is CH₃, Rₐ₂ and R_{b2} are CH₃, Rₐ₃ and R_{b3} are

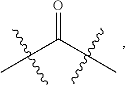 [SMILES: *C(*)=O]

and Rₐ₄ and R_{b4} are

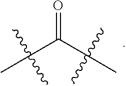 [SMILES: *C(*)=O]

In some variations of formula (12) described in the paragraphs above in which l₁ is 4, Rₐ₁ is H, R_{b1} is CH₃, Rₐ₂ is H, R_{b2} is CH₃, Rₐ₃ and R_{b3} are

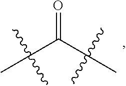 [SMILES: *C(*)=O]

and Rₐ₄ and R_{b4} are

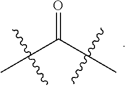 [SMILES: *C(*)=O]

In some variations of formula (12) described in the paragraphs above in which l₁ is 4, Rₐ₁ and R_{b1} are H, Rₐ₂ and R_{b2} are

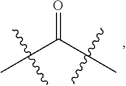 [SMILES: *C(*)=O]

Rₐ₃ and R_{b3} are

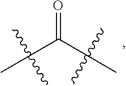 [SMILES: *C(*)=O]

and Rₐ₄ and R_{b4} are

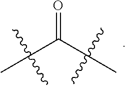 [SMILES: *C(*)=O]

In some variations of formula (12) described in the paragraphs above in which l₁ is 4, Rₐ₁ and R_{b1} are CH₃, Rₐ₂ and R_{b2} are

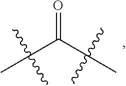 [SMILES: *C(*)=O]

Rₐ₃ and R_{b3} are

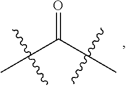 [SMILES: *C(*)=O]

and Rₐ₄ and R_{b4} are

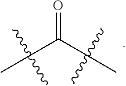 [SMILES: *C(*)=O]

In some variations of formula (12) described in the paragraphs above in which l₁ is 4, Rₐ₁ is H, R_{b1} is CH₃, Rₐ₂ and R_{b2} are

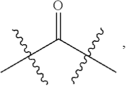 [SMILES: *C(*)=O]

Rₐ₃ and R_{b3} are

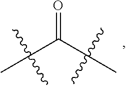 [SMILES: *C(*)=O]

and Rₐ₄ and R_{b4} are

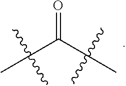 [SMILES: *C(*)=O]

In some variations of formula (12) described in the paragraphs above in which l₁ is 4, Rₐ₁ and R_{b1} are

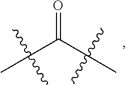 [SMILES: *C(*)=O]

Rₐ₂ and R_{b2} are

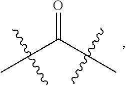 [SMILES: *C(*)=O]

Rₐ₃ and R_{b3}

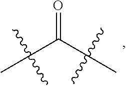 [SMILES: *C(*)=O]

are and Rₐ₄ and R_{b4} are

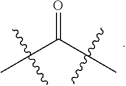 [SMILES: *C(*)=O]

In some variations of formula (12) described in the paragraphs above in which l₁ is 4, Rₐ₁ and R_{b1} are

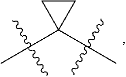 [SMILES: *C1(*)CC1]

Rₐ₂ and R_{b2} are H, Rₐ₃ and R_{b3} are H, and Rₐ₄ and R_{b4} are

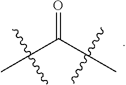 [SMILES: *C(*)=O]

In some variations of formula (12) described in the paragraphs above in which l₁ is 4, Rₐ₁ and R_{b1} are

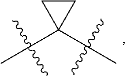 [SMILES: *C1(*)CC1]

Rₐ₂ and R_{b2} are CH₃, Rₐ₃ and R_{b3} are CH₃, and Rₐ₄ and R_{b4} are

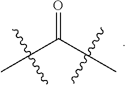 [SMILES: *C(*)=O]

In some variations of formula (12) described in the paragraphs above in which l₁ is 4, Rₐ₁ and R_{b1} are

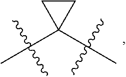 [SMILES: *C1(*)CC1]

Rₐ₂ and R_{b2} are H, Rₐ₃ and R_{b3} are CH₃, and Rₐ₄ and R_{b4} are

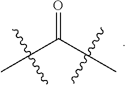 [SMILES: *C(*)=O]

In some variations of formula (12) described in the paragraphs above in which l₁ is 4, Rₐ₁ and R_{b1} are

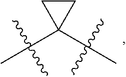 [SMILES: *C1(*)CC1]

Rₐ₂ and R_{b2} are

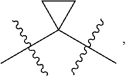 [SMILES: *C1(*)CC1]

Rₐ₃ and R_{b3} are H, and Rₐ₄ and R_{b4} are

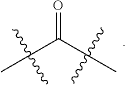 [SMILES: *C(*)=O]

In some variations of formula (12) described in the paragraphs above in which l₁ is 4, Rₐ₁ and R_{b1} are

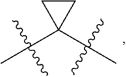 [SMILES: *C1(*)CC1]

Rₐ₂ and R_{b2} are

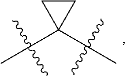 [SMILES: *C1(*)CC1]

Rₐ₃ and R_{b3} are CH₃, and Rₐ₄ and R_{b4} are

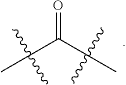 [SMILES: *C(*)=O]

In some variations of formula (12) described in the paragraphs above in which l₁ is 4, Rₐ₁ and R_{b1} are

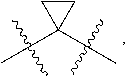 [SMILES: *C1(*)CC1]

Rₐ₂ and R_{b2} are

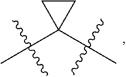 [SMILES: *C1(*)CC1]

Rₐ₃ and R_{b3} are CH₃, and Rₐ₄ and R_{b4} are

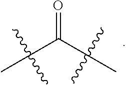 [SMILES: *C(*)=O]

In some variations of formula (12) described in the paragraphs above in which l₁ is 4, Rₐ₁ and R_{b1} are

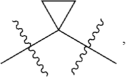 [SMILES: *C1(*)CC1]

Rₐ₂ and R_{b2} are

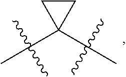 [SMILES: *C1(*)CC1]

Rₐ₃ and R_{b3} are H, and Rₐ₄ and R_{b4} are

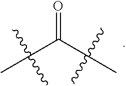 [SMILES: *C(*)=O]

In some variations of formula (12) described in the paragraphs above in which l₁ is 4, Rₐ₁ and R_{b1} are

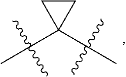 [SMILES: *C1(*)CC1]

Rₐ₂ and R_{b2} are

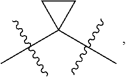 [SMILES: *C1(*)CC1]

Rₐ₃ and R_{b3} are

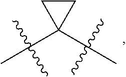 [SMILES: *C1(*)CC1]

and Rₐ₄ and R_{b4} are

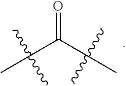 [SMILES: *C(*)=O]

In some variations of formula (12) described in the paragraphs above in which l₁ is 4, Rₐ₁ and R_{b1} are

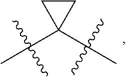 [SMILES: *C1(*)CC1]

Rₐ₂ and R_{b2} are

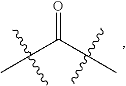 [SMILES: *C(*)=O]

Rₐ₃ and R_{b3} are H, and Rₐ₄ and R_{b4} are

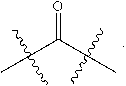 [SMILES: *C(*)=O]

In some variations of formula (12) described in the paragraphs above in which l₁ is 4, Rₐ₁ and R_{b1} are

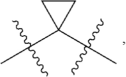 [SMILES: *C1(*)CC1]

Rₐ₂ and R_{b2} are

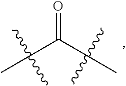 [SMILES: *C(*)=O]

Rₐ₃ and R_{b3} are CH₃, and Rₐ₄ and R_{b4} are

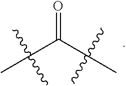 [SMILES: *C(*)=O]

In some variations of formula (12) described in the paragraphs above in which l₁ is 4, Rₐ₁ and R_{b1} are

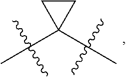 [SMILES: *C1(*)CC1]

Rₐ₂ and R_{b2} are

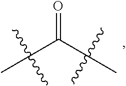 [SMILES: *C(*)=O]

Rₐ₃ and R_{b3} are CH₃, and Rₐ₄ and R_{b4} are

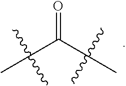 [SMILES: *C(*)=O]

In some variations of formula (12) described in the paragraphs above in which l₁ is 4, Rₐ₁ and R_{b1} are

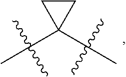 [SMILES: *C1(*)CC1]

Rₐ₂ and R_{b2} are

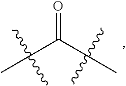 [SMILES: *C(*)=O]

Rₐ₃ and R_{b3} are H, and Rₐ₄ and R_{b4} are

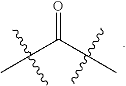 [SMILES: *C(*)=O]

In some variations of formula (12) described in the paragraphs above in which l₁ is 4, Rₐ₁ and R_{b1} are

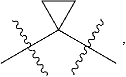 [SMILES: *C1(*)CC1]

Rₐ₂ and R_{b2} are

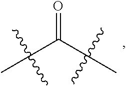 [SMILES: *C(*)=O]

Rₐ₃ and R_{b3} are

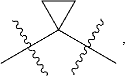 [SMILES: *C1(*)CC1]

and Rₐ₄ and R_{b4} are

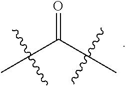 [SMILES: *C(*)=O]

In some variations of formula (12) described in the paragraphs above in which l₁ is 4, Rₐ₁ and R_{b1} are

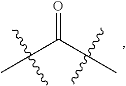 [SMILES: *C(*)=O]

Rₐ₂ and R_{b2} are

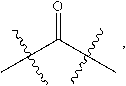 [SMILES: *C(*)=O]

Rₐ₃ and R_{b3} are H, and Rₐ₄ and R_{b4} are

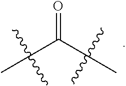 [SMILES: *C(*)=O]

In some variations of formula (12) described in the paragraphs above in which l₁ is 4, Rₐ₁ and R_{b1} are

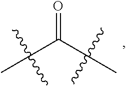 [SMILES: *C(*)=O]

Rₐ₂ and R_{b2} are

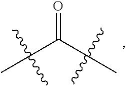 [SMILES: *C(*)=O]

Rₐ₃ and R_{b3} are CH₃, and Rₐ₄ and R_{b4} are

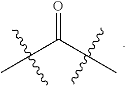 [SMILES: *C(*)=O]

In some variations of formula (12) described in the paragraphs above in which l₁ is 4, Rₐ₁ and R_{b1} are

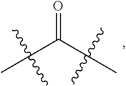 [SMILES: *C(*)=O]

Rₐ₂ and R_{b2} are

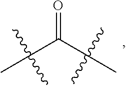 [SMILES: *C(*)=O]

Rₐ₃ and R_{b3} are CH₃, and Rₐ₄ and R_{b4} are

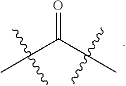 [SMILES: *C(*)=O]

In some variations of formula (12) described in the paragraphs above in which l₁ is 4, Rₐ₁ and R_{b1} are

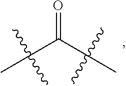 [SMILES: *C(*)=O]

Rₐ₂ and R_{b2} are

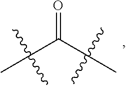 [SMILES: *C(*)=O]

Rₐ₃ and R_{b3} are H, and Rₐ₄ and R_{b4} are

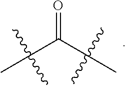 [SMILES: *C(*)=O]

In some variations of formula (12) described in the paragraphs above in which l₁ is 4, Rₐ₁ and R_{b1} are

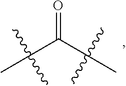 [SMILES: *C(*)=O]

Rₐ₂ and R_{b2} are

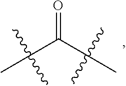 [SMILES: *C(*)=O]

Rₐ₃ and R_{b3} are

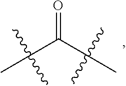 [SMILES: *C(*)=O]

and Rₐ₄ and R_{b4} are

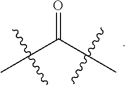 [SMILES: *C(*)=O]

In some variations of formula (12) described in the paragraphs above in which l₁ is 4, Rₐ₁ and R_{b1} are H, Rₐ₂ and R_{b2} are H, and Rₐ₃ and R_{b3} and Rₐ₄ and R_{b4} together are

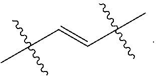 [SMILES: */C=C/*]

In some variations of formula (12) described in the paragraphs above in which l₁ is 4, Rₐ₁ and R_{b1} are H, Rₐ₂ and R_{b2} are CH₃, and Rₐ₃ and R_{b3} and Rₐ₄ and R_{b4} together are

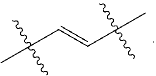 [SMILES: */C=C/*]

In some variations of formula (12) described in the paragraphs above in which l₁ is 4, Rₐ₁ and Rₐ₂ are H, R_{b1} and R_{b2} are CH₃, and Rₐ₃ and R_{b3} and Rₐ₄ and R_{b4} together are

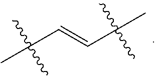 [SMILES: */C=C/*]

In some variations of formula (12) described in the paragraphs above in which l₁ is 4, Rₐ₁ and R_{b1} are H, Rₐ₂ and R_{b2} are

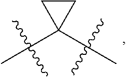 [SMILES: *C1(*)CC1]

and Rₐ₃ and R_{b3} and Rₐ₄ and R_{b4} together are

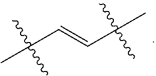 [SMILES: */C=C/*]

In some variations of formula (12) described in the paragraphs above in which l₁ is 4, Rₐ₁ and R_{b1} are

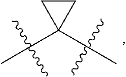 [SMILES: *C1(*)CC1]

Rₐ₂ and R_{b2} are CH₃, and Rₐ₃ and R_{b3} and Rₐ₄ and R_{b4} together are

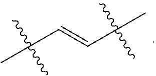 [SMILES: */C=C/*]

In some variations of formula (12) described in the paragraphs above in which l₁ is 4, Rₐ₁ is H, R_{b1} is CH₃, Rₐ₂ and R_{b2} are

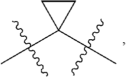 [SMILES: *C1(*)CC1]

and Rₐ₃ and R_{b3} and Rₐ₄ and R_{b4} together are

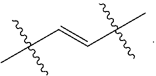 [SMILES: */C=C/*]

In some variations of formula (12) described in the paragraphs above in which l₁ is 4, Rₐ₁ and R_{b1} are H, Rₐ₂ and R_{b2} are

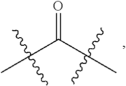 [SMILES: *C(*)=O]

and Rₐ₃ and R_{b3} and Rₐ₄ and R_{b4} together are

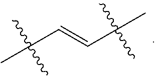 [SMILES: */C=C/*]

In some variations of formula (12) described in the paragraphs above in which l₁ is 4, Rₐ₁ and R_{b1} are

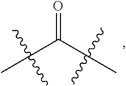 [SMILES: *C(*)=O]

Rₐ₂ and R_{b2} are CH₃, and Rₐ₃ and R_{b3} and Rₐ₄ and R_{b4} together are

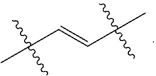 [SMILES: */C=C/*]

In some variations of formula (12) described in the paragraphs above in which l₁ is 4, Rₐ₁ is H, R_{b1} is CH₃, Rₐ₂ and R_{b2} are

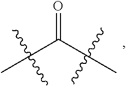 [SMILES: *C(*)=O]

and Rₐ₃ and R_{b3} and Rₐ₄ and R_{b4} together are

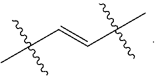 [SMILES: */C=C/*]

In some variations of formula (12) described in the paragraphs above in which l₁ is 4, Rₐ₁ and R_{b1} are

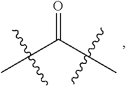 [SMILES: *C(*)=O]

Rₐ₂ and R_{b2} are

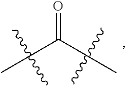 [SMILES: *C(*)=O]

and Rₐ₃ and R_{b3} and Rₐ₄ and R_{b4} together are

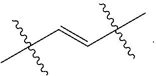 [SMILES: */C=C/*]

In some variations of formula (12) described in the paragraphs above in which l₁ is 4, Rₐ₁ and R_{b1} are

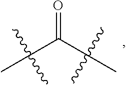 [SMILES: *C(*)=O]

Rₐ₂ and R_{b2} are

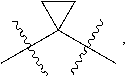 [SMILES: *C1(*)CC1]

and Rₐ₃ and R_{b3} and Rₐ₄ and R_{b4} together are

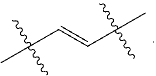 [SMILES: */C=C/*]

In some variations of formula (12) described in the paragraphs above in which l₁ is 4, Rₐ₁ and R_{b1} are

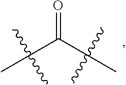 [SMILES: *C(*)=O]

Rₐ₂ and R_{b2} are

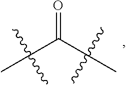 [SMILES: *C(*)=O]

and Rₐ₃ and R_{b3} and Rₐ₄ and R_{b4} together are

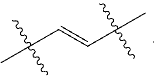 [SMILES: */C=C/*]

In some variations of formula (12) described in the paragraphs above in which l₁ is 4, Rₐ₁ and R_{b1} are

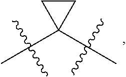 [SMILES: *C1(*)CC1]

Rₐ₂ and R_{b2} are

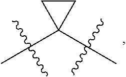 [SMILES: *C1(*)CC1]

and Rₐ₃ and R_{b3} and Rₐ₄ and R_{b4} together are

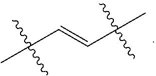 [SMILES: */C=C/*]

In some variations of formula (12) described in the paragraphs above in which l₁ is 4, Rₐ₁ and R_{b1} and Rₐ₂ and R_{b2} together are

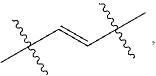 [SMILES: */C=C/*]

and Rₐ₁ and R_{b3} and Rₐ₄ and R_{b4} together are

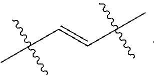 [SMILES: */C=C/*]

In some variations of formula (12) described in the paragraphs above in which l₂ is 1, Rₐ₁ and R_{b1} are both H. In some variations of formula (12) described in the paragraphs above in which l₂ is 1, Rₐ₁ is H and R_{b1} is CH₃. In some variations of formula (12) described in the paragraphs above in which l₂ is 1, Rₐ₁ and R_{b1} are both CH₃. In some variations of formula (12) described in the paragraphs above in which l₂ is 1, Rₐ₁ and R_{b1} are

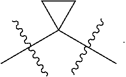 [SMILES: *C1(*)CC1]

In some variations of formula (12) described in the paragraphs above in which l₂ is 1, Rₐ₁ and R_{b1} are

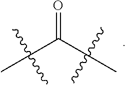 [SMILES: *C(*)=O]

In variations of formula (12) described in the paragraphs above in which l₂ is 2, the two instances of Rₐ and R_{b} are indicated as Rₐ₁ and R_{b1} and Rₐ₂ and R_{b2}, respectively. In some variations of formula (12) described in the paragraphs above in which l₂ is 2, each of Rₐ₁ and R_{b1} and Rₐ₂ and R_{b2} is H. In some variations of formula (12) described in the paragraphs above in which l₂ is 2, each of Rₐ₁ and R_{b1} and Rₐ₂ and R_{b2} is CH₃. In some variations of formula (12) described in the paragraphs above in which l₂ is 2, each of Rₐ₁ and Rₐ₂ is H and each of R_{b1} and R_{b2} is CH₃. In some variations of formula (12) described in the paragraphs above in which l₂ is 2, each of Rₐ₁, Rₐ₂, and R_{b1} is H and R_{b2} is CH₃.

In some variations of formula (12) described in the paragraphs above in which l₂ is 2, each of Rₐ₁ and R_{b1} is H and Rₐ₂ and R_{b2} are

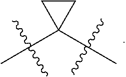 [SMILES: *C1(*)CC1]

In some variations of formula (12) described in the paragraphs above in which l₂ is 2, each of Rₐ₁ and R_{b1} is H and Rₐ₂ and R_{b2} are

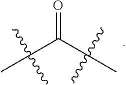 [SMILES: *C(*)=O]

In some variations of formula (12) described in the paragraphs above in which l₂ is 2, each of Rₐ₁ and R_{b1} is CH₃ and Rₐ₂ and R_{b2} are

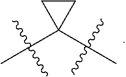 [SMILES: *C1(*)CC1]

In some variations of formula (12) described in the paragraphs above in which l₂ is 2, each of Rₐ₁ and R_{b1} is CH₃ and Rₐ₂ and R_{b2} are

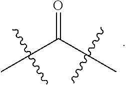 [SMILES: *C(*)=O]

In some variations of formula (12) described in the paragraphs above in which l₂ is 2, Rₐ₁ is H, R_{b1} is CH₃, and Rₐ₂ and R_{b2} are

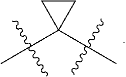 [SMILES: *C1(*)CC1]

In some variations of formula (12) described in the paragraphs above in which l₂ is 2, Rₐ₁ is H, R_{b1} is CH₃, and Rₐ₂ and R_{b2} are

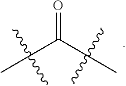 [SMILES: *C(*)=O]

In some variations of formula (12) described in the paragraphs above in which l₂ is 2, Rₐ₁ and R_{b1} and Rₐ₂ and R_{b2} together are

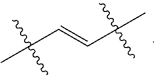 [SMILES: */C=C/*]

In some variations of formula (12) described in the paragraphs above in which l₂ is 2, Rₐ₁ and R_{b1} are

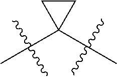 [SMILES: *C1(*)CC1]

Rₐ₂ and R_{b2} are

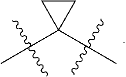 [SMILES: *C1(*)CC1]

In some variations of formula (12) described in the paragraphs above in which l₂ is 2, Rₐ₁ and R_{b1} are

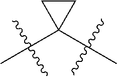 [SMILES: *C1(*)CC1]

Rₐ₂ and R_{b2} are

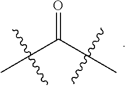 [SMILES: *C(*)=O]

In some variations of formula (12) described in the paragraphs above in which l₂ is 2, Rₐ₁ and R_{b1} are

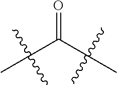 [SMILES: *C(*)=O]

Rₐ₂ and R_{b2} are

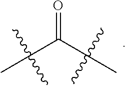 [SMILES: *C(*)=O]

In variations of formula (12) described in the paragraphs above in which l₂ is 3, the three instances of Rₐ and R_{b} are indicated as Rₐ₁ and R_{b1}; Rₐ₂ and R_{b2}; and Rₐ₃ and R_{b3}, respectively. In some variations of formula (12) described in the paragraphs above in which l₂ is 3, In some variations of formula (12) described in the paragraphs above in which l₂ is 3, each of Rₐ₁, R_{b1}, Rₐ₂, R_{b2}, Rₐ₃, and R_{b3} is H. In some variations of formula (12) described in the paragraphs above in which l₂ is 3, each of Rₐ₁, R_{b1}, Rₐ₂, R_{b2}, Rₐ₃, and R_{b3} is CH₃. In some variations of formula (12) described in the paragraphs above in which l₂ is 3, each of Rₐ₁, R_{b1}, Rₐ₂, and R_{b2}, is H and each of Rₐ₃ and R_{b3} is CH₃. In some variations of formula (12) described in the paragraphs above in which l₂ is 3, each of Rₐ₁, R_{b1}, Rₐ₂, and R_{b2}, is CH₃ and each of Rₐ₃ and R_{b3} is H. In some variations of formula (12) described in the paragraphs above in which l₂ is 3, Rₐ₁ is H and each of Rₐ₂, Rₐ₃, R_{b1}, R_{b2}, and R_{b3} is CH₃. In some variations of formula (12) described in the paragraphs above in which l₂ is 3, each of Rₐ₁ and Rₐ₂ is H and each of Rₐ₃, R_{b1}, R_{b2}, an R_{b3} is CH₃. In some variations of formula (12) described in the paragraphs above in which l₂ is 3, each of Rₐ₁, Rₐ₂, and Rₐ₃ is H and each of R_{b1}, R_{b2}, an R_{b3} is CH₃.

In some variations of formula (12) described in the paragraphs above in which l₂ is 3, each of Rₐ₁, R_{b1}, Rₐ₂, and R_{b2}, is H and Rₐ₃ and R_{b3} are

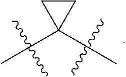 [SMILES: *C1(*)CC1]

In some variations of formula (12) described in the paragraphs above in which l₂ is 3, each of Rₐ₁, R_{b1}, Rₐ₂, and R_{b2}, is CH₃ and Rₐ₃ and R_{b3} are

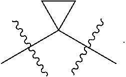 [SMILES: *C1(*)CC1]

In some variations of formula (12) described in the paragraphs above in which l₂ is 3, Rₐ₁ is H and each of Rₐ₂, R_{b1}, and R_{b2} is CH₃, and Rₐ₃ and R_{b3} are

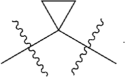 [SMILES: *C1(*)CC1]

In some variations of formula (12) described in the paragraphs above in which l₂ is 3, each of Rₐ₁ and Rₐ₂ is H and each of R_{b1} and R_{b2} is CH₃, and Rₐ₃ and R_{b3} are

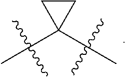 [SMILES: *C1(*)CC1]

In some variations of formula (12) described in the paragraphs above in which l₂ is 3, each of Rₐ₁, R_{b1}, Rₐ₂, and R_{b2}, is H and Rₐ₃ and R_{b3} are

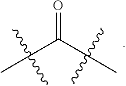 [SMILES: *C(*)=O]

In some variations of formula (12) described in the paragraphs above in which l₂ is 3, each of Rₐ₁, R_{b1}, Rₐ₂, and R_{b2}, is CH₃ and Rₐ₃ and R_{b3} are

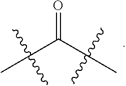 [SMILES: *C(*)=O]

In some variations of formula (12) described in the paragraphs above in which l₂ is 3, Rₐ₁ is H and each of Rₐ₂, R_{b1}, and R_{b2} is CH₃, and Rₐ₃ and R_{b3} are

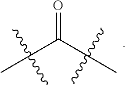 [SMILES: *C(*)=O]

In some variations of formula (12) described in the paragraphs above in which l₂ is 3, each of Rₐ₁ and Rₐ₂ is H and each of R_{b1} and R_{b2} is CH₃, and Rₐ₃ and R_{b3} are

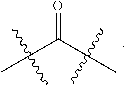 [SMILES: *C(*)=O]

In some variations of formula (12) described in the paragraphs above in which l₂ is 3, Rₐ₁ and R_{b1} are

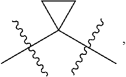 [SMILES: *C1(*)CC1]

Rₐ₂ and R_{b2} are

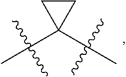 [SMILES: *C1(*)CC1]

and each of Rₐ₃ and R_{b3} is H. In some variations of formula (12) described in the paragraphs above in which l₂ is 3, Rₐ₁ and R_{b1} are

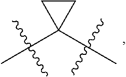 [SMILES: *C1(*)CC1]

Rₐ₂ and R_{b2} are

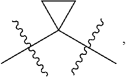 [SMILES: *C1(*)CC1]

and each of Rₐ₃ and R_{b3} is CH₃. In some variations of formula (12) described in the paragraphs above in which l₂ is 3, Rₐ₁ and R_{b1} are

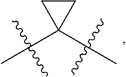 [SMILES: *C1(*)CC1]

Rₐ₂ and R_{b2} are

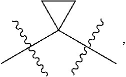 [SMILES: *C1(*)CC1]

Rₐ₃ is H, R_{b3} is CH₃. In some variations of formula (12) described in the paragraphs above in which l₂ is 3, Rₐ₁ and R_{b1} are

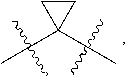 [SMILES: *C1(*)CC1]

Rₐ₂ and R_{b2} are

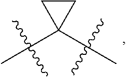 [SMILES: *C1(*)CC1]

and each of Rₐ₃ and R_{b3} is H. In some variations of formula (12) described in the paragraphs above in which l₂ is 3, Rₐ₁ and R_{b1} are

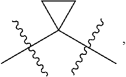 [SMILES: *C1(*)CC1]

Rₐ₂ and R_{b2} are

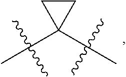 [SMILES: *C1(*)CC1]

and each of Rₐ₃ and R_{b3} is CH₃. In some variations of formula (12) described in the paragraphs above in which l₂ is 3, Rₐ₁ and R_{b1} are

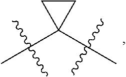 [SMILES: *C1(*)CC1]

Rₐ₂ and R_{b2} are

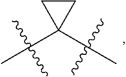 [SMILES: *C1(*)CC1]

Rₐ₃ is H, R_{b3} is CH₃.

In some variations of formula (12) described in the paragraphs above in which l₂ is 3, Rₐ₁ and R_{b1} are

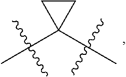 [SMILES: *C1(*)CC1]

Rₐ₂ and R_{b2} are

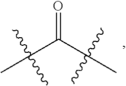 [SMILES: *C(*)=O]

and each of Rₐ₃ and R_{b3} is H. In some variations of formula (12) described in the paragraphs above in which l₂ is 3, Rₐ₁ and R_{b1} are

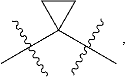 [SMILES: *C1(*)CC1]

Rₐ₂ and R_{b2} are

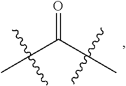 [SMILES: *C(*)=O]

and each of Rₐ₃ and R_{b3} is CH₃. In some variations of formula (12) described in the paragraphs above in which l₂ is 3, Rₐ₁ and R_{b1} are

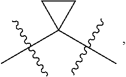 [SMILES: *C1(*)CC1]

Rₐ₂ and R_{b2} are

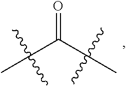 [SMILES: *C(*)=O]

Rₐ₃ is H, and R_{b3} is CH₃. In some variations of formula (12) described in the paragraphs above in which l₂ is 3, Rₐ₁ and R_{b1} are

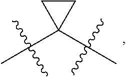 [SMILES: *C1(*)CC1]

Rₐ₂ and R_{b2} are

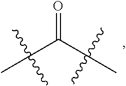 [SMILES: *C(*)=O]

and each of Rₐ₃ and R_{b3} is H. In some variations of formula (12) described in the paragraphs above in which l₂ is 3, Rₐ₁ and R_{b1} are

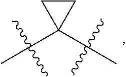 [SMILES: *C1(*)CC1]

Rₐ₂ and R_{b2} are

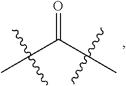 [SMILES: *C(*)=O]

and each of Rₐ₃ and R_{b3} is CH₃. In some variations of formula (12) described in the paragraphs above in which l₂ is 3, Rₐ₁ and R_{b1} are

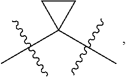 [SMILES: *C1(*)CC1]

Rₐ₂ and R_{b2} are

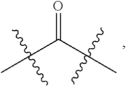 [SMILES: *C(*)=O]

Rₐ₃ is H, and R_{b3} is CH₃.

In some variations of formula (12) described in the paragraphs above in which l₂ is 3, Rₐ₁ and R_{b1} are

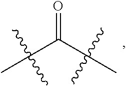 [SMILES: *C(*)=O]

Rₐ₂ and R_{b2} are

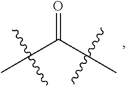 [SMILES: *C(*)=O]

and each of Rₐ₃ and R_{b3} is H. In some variations of formula (12) described in the paragraphs above in which l₂ is 3, Rₐ₁ and R_{b1} are

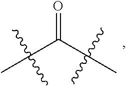 [SMILES: *C(*)=O]

Rₐ₂ and R_{b2} are

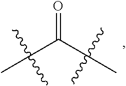 [SMILES: *C(*)=O]

and each of Rₐ₃ and R_{b3} is CH₃. In some variations of formula (12) described in the paragraphs above in which l₂ is 3, Rₐ₁ and R_{b1} are

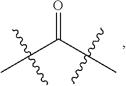 [SMILES: *C(*)=O]

Rₐ₂ and R_{b2} are

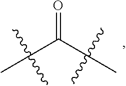 [SMILES: *C(*)=O]

Rₐ₃ is H, and R_{b3} is CH₃. In some variations of formula (12) described in the paragraphs above in which l₂ is 3, Rₐ₁ and R_{b1} are

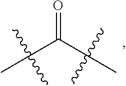 [SMILES: *C(*)=O]

Rₐ₂ and R_{b2} are

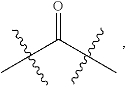 [SMILES: *C(*)=O]

and each of Rₐ₃ and R_{b3} is H. In some variations of formula (12) described in the paragraphs above in which l₂ is 3, Rₐ₁ and R_{b1} are

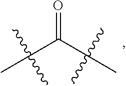 [SMILES: *C(*)=O]

Rₐ₂ and R_{b2} are

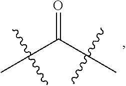 [SMILES: *C(*)=O]

and each of Rₐ₃ and R_{b3} is CH₃. In some variations of formula (12) described in the paragraphs above in which l₂ is 3, Rₐ₁ and R_{b1} are

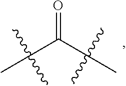 [SMILES: *C(*)=O]

Rₐ₂ and R_{b2} are

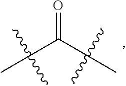 [SMILES: *C(*)=O]

Rₐ₃ is H, and R_{b3} is CH₃.

In some variations of formula (12) described in the paragraphs above in which l₂ is 3, Rₐ₁ and R_{b1} and Rₐ₂ and R_{b2} together are

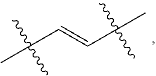 [SMILES: */C=C/*]

and each of Rₐ₃ and R_{b3} is H. In some variations of formula (12) described in the paragraphs above in which l₂ is 3, Rₐ₁ and R_{b1} and Rₐ₂ and R_{b2} together are

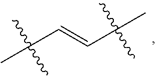 [SMILES: */C=C/*]

and each of Rₐ₃ and R_{b3} is CH₃. In some variations of formula (12) described in the paragraphs above in which l₂ is 3, Rₐ₁ and R_{b1} and Rₐ₂ and R_{b2} together are

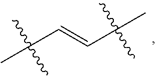 [SMILES: */C=C/*]

Rₐ₃ is H, and R_{b3} is CH₃. In some variations of formula (12) described in the paragraphs above in which l₂ is 3, Rₐ₁ and R_{b1} and Rₐ₂ and R_{b2} together are

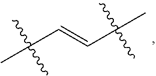 [SMILES: */C=C/*]

and Rₐ₃ and R_{b3} are

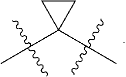 [SMILES: *C1(*)CC1]

In some variations of formula (12) described in the paragraphs above in which l₂ is 3, Rₐ₁ and R_{b1} and Rₐ₂ and R_{b2} together are

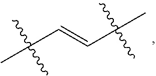 [SMILES: */C=C/*]

and Rₐ₃ and R_{b3} are

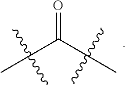 [SMILES: *C(*)=O]

In variations of formula (12) described in the paragraphs above in which l₂ is 4, the three instances of Rₐ and R_{b} are indicated as Rₐ₁ and R_{b1}; Rₐ₂ and R_{b2}; Rₐ₃ and R_{b3}; and Rₐ₄ and R_{b4}, respectively. In some variations of formula (12) described in the paragraphs above in which l₂ is 4, In some variations of formula (12) described in the paragraphs above in which l₂ is 4, each of Rₐ₁, R_{b1}, Rₐ₂, R_{b2}, Rₐ₃, R_{b3}, Rₐ₄, and R_{b4} is H. In some variations of formula (12) described in the paragraphs above in which l₂ is 4, each of Rₐ₁, R_{b1}, Rₐ₂, R_{b2}, Rₐ₃, R_{b3}, Rₐ₄, and R_{b4} is CH₃. In some variations of formula (12) described in the paragraphs above in which l₂ is 4, each of Rₐ₁, R_{b1}, Rₐ₂, R_{b2}, Rₐ₃, and R_{b3} is H and each of Rₐ₄ and R_{b4} is CH₃. In some variations of formula (12) described in the paragraphs above in which l₂ is 4, each of Rₐ₁, R_{b1}, Rₐ₂, and R_{b2} is H and each of Rₐ₃, R_{b3}, Rₐ₄, and R_{b4} is CH₃. In some variations of formula (12) described in the paragraphs above in which l₂ is 4, each of Rₐ₁, R_{b1}, Rₐ₂, R_{b2}, Rₐ₃, and R_{b3} is CH₃ and each of Rₐ₄ and R_{b4} is H.

In some variations of formula (12) described in the paragraphs above in which l₂ is 4, each of Rₐ₁, Rₐ₂, and Rₐ₃ is H and each of R_{b1}, R_{b2}, and R_{b3} is CH₃. In some variations of formula (12) described in the paragraphs above in which l₂ is 4, Rₐ₁ is H, R_{b1} is CH₃, and each of Rₐ₂, R_{b2}, Rₐ₃, and R_{b3} is CH₃. In some variations of formula (12) described in the paragraphs above in which l₂ is 4, Rₐ₁ is H, R_{b1} is CH₃, and each of Rₐ₂, R_{b2}, Rₐ₃, and R_{b3} is H. In some variations of formula (12) described in the paragraphs above in which l₂ is 4, each of Rₐ₁ and Rₐ₂ is H, each of R_{b1} and R_{b2} is CH₃, and each of Rₐ₃ and R_{b3} is H. In some variations of formula (12) described in the paragraphs above in which l₂ is 4, each of Rₐ₁ and Rₐ₂ is H, each of R_{b1} and R_{b2} is CH₃, and each of Rₐ₃ and R_{b3} is CH₃.

In some variations of formula (12) described in the paragraphs above in which l₂ is 4, Rₐ₁ and R_{b1} are H, Rₐ₂ and R_{b2} are H, Rₐ₃ and R_{b3} are H, and Rₐ₄ and R_{b4} are

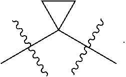 [SMILES: *C1(*)CC1]

In some variations of formula (12) described in the paragraphs above in which l₂ is 4, Rₐ₁ and R_{b1} are H, Rₐ₂ and R_{b2} are H, Rₐ₃ and R_{b3} are CH₃, and Rₐ₄ and R_{b4} are

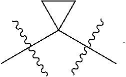 [SMILES: *C1(*)CC1]

In some variations of formula (12) described in the paragraphs above in which l₂ is 4, Rₐ₁ and R_{b1} are H, Rₐ₂ and R_{b2} are CH₃, Rₐ₃ and R_{b3} are CH₃, and Rₐ₄ and R_{b4} are

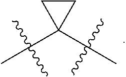 [SMILES: *C1(*)CC1]

In some variations of formula (12) described in the paragraphs above in which l₂ is 4, Rₐ₁ and R_{b1} are CH₃, Rₐ₂ and R_{b2} are CH₃, Rₐ₃ and R_{b3} are CH₃, and Rₐ₄ and R_{b4} are

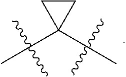 [SMILES: *C1(*)CC1]

In some variations of formula (12) described in the paragraphs above in which l₂ is 4, Rₐ₁ and R_{b1} are H, Rₐ₂ and R_{b2} are H, Rₐ₃ and R_{b3} are

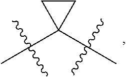 [SMILES: *C1(*)CC1]

and Rₐ₄ and R_{b4} are

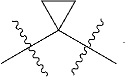 [SMILES: *C1(*)CC1]

In some variations of formula (12) described in the paragraphs above in which l₂ is 4, Rₐ₁ and R_{b1} are H, Rₐ₂ and R_{b2} are CH₃, Rₐ₃ and R_{b3} are

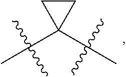 [SMILES: *C1(*)CC1]

and Rₐ₄ and R_{b4} are

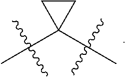 [SMILES: *C1(*)CC1]

In some variations of formula (12) described in the paragraphs above in which l₂ is 4, Rₐ₁ and R_{b1} are CH₃, Rₐ₂ and R_{b2} are CH₃, Rₐ₃ and R_{b3} are

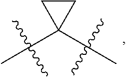 [SMILES: *C1(*)CC1]

and Rₐ₄ and R_{b4} are

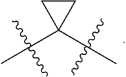 [SMILES: *C1(*)CC1]

In some variations of formula (12) described in the paragraphs above in which l₂ is 4, Rₐ₁ is H, R_{b1} is CH₃, Rₐ₂ and R_{b2} are CH₃, Rₐ₃ and R_{b3} are

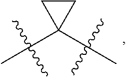 [SMILES: *C1(*)CC1]

and Rₐ₄ and R_{b4} are

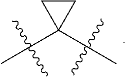 [SMILES: *C1(*)CC1]

In some variations of formula (12) described in the paragraphs above in which l₂ is 4, Rₐ₁ is H, R_{b1} is CH₃, Rₐ₂ is H, R_{b2} is CH₃, Rₐ₃ and R_{b3} are

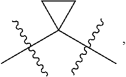 [SMILES: *C1(*)CC1]

and Rₐ₄ and R_{b4} are

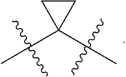 [SMILES: *C1(*)CC1]

In some variations of formula (12) described in the paragraphs above in which l₂ is 4, Rₐ₁ and R_{b1} are H, Rₐ₂ and R_{b2} are

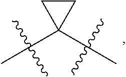 [SMILES: *C1(*)CC1]

Rₐ₃ and R_{b3} are

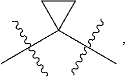 [SMILES: *C1(*)CC1]

and Rₐ₄ and R_{b4} are

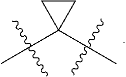 [SMILES: *C1(*)CC1]

In some variations of formula (12) described in the paragraphs above in which l₂ is 4, Rₐ₁ and R_{b1} are CH₃, Rₐ₂ and R_{b2} are

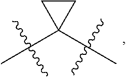 [SMILES: *C1(*)CC1]

Rₐ₃ and R_{b3} are

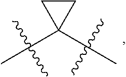 [SMILES: *C1(*)CC1]

and Rₐ₄ and R_{b4} are

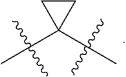 [SMILES: *C1(*)CC1]

In some variations of formula (12) described in the paragraphs above in which l₂ is 4, Rₐ₁ is H, R_{b1} is CH₃, Rₐ₂ and R_{b2} are

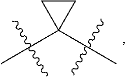 [SMILES: *C1(*)CC1]

Rₐ₃ and R_{b3} are

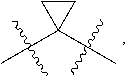 [SMILES: *C1(*)CC1]

and Rₐ₄ and R_{b4} are

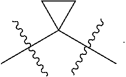 [SMILES: *C1(*)CC1]

In some variations of formula (12) described in the paragraphs above in which l₂ is 4, Rₐ₁ and R_{b1} are

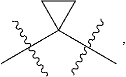 [SMILES: *C1(*)CC1]

Rₐ₂ and R_{b2} are

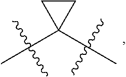 [SMILES: *C1(*)CC1]

Rₐ₃ and R_{b3} are

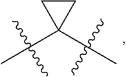 [SMILES: *C1(*)CC1]

and Rₐ₄ and R_{b4} are

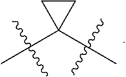 [SMILES: *C1(*)CC1]

In some variations of formula (12) described in the paragraphs above in which l₂ is 4, Rₐ₁ and R_{b1} are H, Rₐ₂ and R_{b2} are H, Rₐ₃ and R_{b3} are H, and Rₐ₄ and R_{b4} are

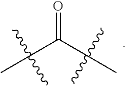 [SMILES: *C(*)=O]

In some variations of formula (12) described in the paragraphs above in which l₂ is 4, Rₐ₁ and R_{b1} are H, Rₐ₂ and R_{b2} are H, Rₐ₃ and R_{b3} are CH₃, and Rₐ₄ and R_{b4} are

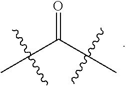 [SMILES: *C(*)=O]

In some variations of formula (12) described in the paragraphs above in which l₂ is 4, Rₐ₁ and R_{b1} are H, Rₐ₂ and R_{b2} are CH₃, Rₐ₃ and R_{b3} are CH₃, and Rₐ₄ and R_{b4} are

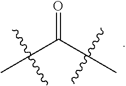 [SMILES: *C(*)=O]

In some variations of formula (12) described in the paragraphs above in which l₂ is 4, Rₐ₁ and R_{b1} are CH₃, Rₐ₂ and R_{b2} are CH₃, Rₐ₃ and R_{b3} are CH₃, and Rₐ₄ and R_{b4} are

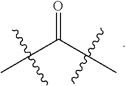 [SMILES: *C(*)=O]

In some variations of formula (12) described in the paragraphs above in which l₂ is 4, Rₐ₁ and R_{b1} are H, Rₐ₂ and R_{b2} are H, Rₐ₃ and R_{b3} are

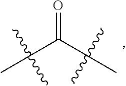 [SMILES: *C(*)=O]

and Rₐ₄ and R_{b4} are v. In some variations of formula (12) described in the paragraphs above in which l₂ is 4, Rₐ₁ and R_{b1} are H, Rₐ₂ and R_{b2} are CH₃, Rₐ₃ and R_{b3} are

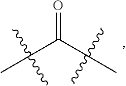 [SMILES: *C(*)=O]

and Rₐ₄ and R_{b4} are

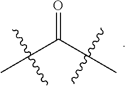 [SMILES: *C(*)=O]

In some variations of formula (12) described in the paragraphs above in which l₂ is 4, Rₐ₁ and R_{b1} are CH₃, Rₐ₂ and R_{b2} are CH₃, Rₐ₃ and R_{b3} are

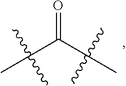 [SMILES: *C(*)=O]

and Rₐ₄ and R_{b4} are

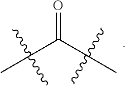 [SMILES: *C(*)=O]

In some variations of formula (12) described in the paragraphs above in which l₂ is 4, Rₐ₁ is H, R_{b1} is CH₃, Rₐ₂ and R_{b2} are CH₃, Rₐ₃ and R_{b3} are

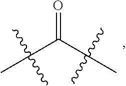 [SMILES: *C(*)=O]

and Rₐ₄ and R_{b4} are

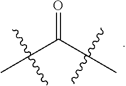 [SMILES: *C(*)=O]

In some variations of formula (12) described in the paragraphs above in which l₂ is 4, Rₐ₁ is H, R_{b1} is CH₃, Rₐ₂ is H, R_{b2} is CH₃, Rₐ₃ and R_{b3} are

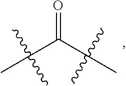 [SMILES: *C(*)=O]

and Rₐ₄ and R_{b4} are

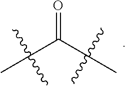 [SMILES: *C(*)=O]

In some variations of formula (12) described in the paragraphs above in which l₂ is 4, Rₐ₁ and R_{b1} are H, Rₐ₂ and R_{b2} are

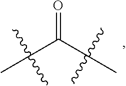 [SMILES: *C(*)=O]

Rₐ₃ and R_{b3} are

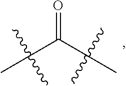 [SMILES: *C(*)=O]

and Rₐ₄ and R_{b4} are

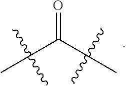 [SMILES: *C(*)=O]

In some variations of formula (12) described in the paragraphs above in which l₂ is 4, Rₐ₁ and R_{b1} are CH₃, Rₐ₂ and R_{b2} are

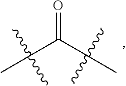 [SMILES: *C(*)=O]

Rₐ₃ and R_{b3} are

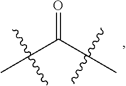 [SMILES: *C(*)=O]

and Rₐ₄ and R_{b4} are

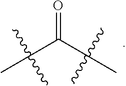 [SMILES: *C(*)=O]

In some variations of formula (12) described in the paragraphs above in which l₂ is 4, Rₐ₁ is H, R_{b1} is CH₃, Rₐ₂ and R_{b2} are

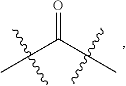 [SMILES: *C(*)=O]

Rₐ₃ and R_{b3} are

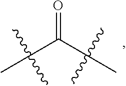 [SMILES: *C(*)=O]

and Rₐ₄ and R_{b4} are

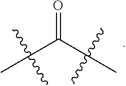 [SMILES: *C(*)=O]

In some variations of formula (12) described in the paragraphs above in which l₂ is 4, Rₐ₁ and R_{b1} are

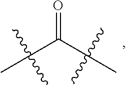 [SMILES: *C(*)=O]

Rₐ₂ and R_{b2} are

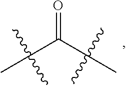 [SMILES: *C(*)=O]

Rₐ₃ and R_{b3} are

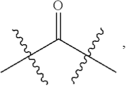 [SMILES: *C(*)=O]

and Rₐ₄ and R_{b4} are

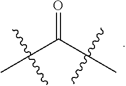 [SMILES: *C(*)=O]

In some variations of formula (12) described in the paragraphs above in which l₂ is 4, Rₐ₁ and R_{b1} are

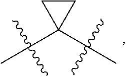 [SMILES: *C1(*)CC1]

Rₐ₂ and R_{b2} are H, Rₐ₃ and R_{b3} are H, and Rₐ₄ and R_{b4} are

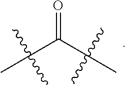 [SMILES: *C(*)=O]

In some variations of formula (12) described in the paragraphs above in which l₂ is 4, Rₐ₁ and R_{b1} are

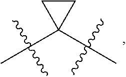 [SMILES: *C1(*)CC1]

Rₐ₂ and R_{b2} are CH₃, Rₐ₃ and R_{b3} are CH₃, and Rₐ₄ and R_{b4} are

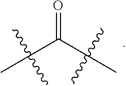 [SMILES: *C(*)=O]

In some variations of formula (12) described in the paragraphs above in which l₂ is 4, Rₐ₁ and R_{b1} are

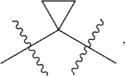 [SMILES: *C1(*)CC1]

Rₐ₂ and R_{b2} are H, Rₐ₃ and R_{b3} are CH₃, and Rₐ₄ and R_{b4} are

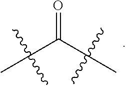 [SMILES: *C(*)=O]

In some variations of formula (12) described in the paragraphs above in which l₂ is 4, Rₐ₁ and R_{b1} are

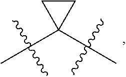 [SMILES: *C1(*)CC1]

Rₐ₂ and R_{b2} are

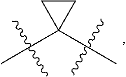 [SMILES: *C1(*)CC1]

Rₐ₃ and R_{b3} are H, and Rₐ₄ and R_{b4} are

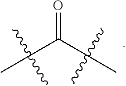 [SMILES: *C(*)=O]

In some variations of formula (12) described in the paragraphs above in which l₂ is 4, Rₐ₁ and R_{b1} are

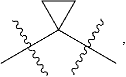 [SMILES: *C1(*)CC1]

Rₐ₂ and R_{b2} are

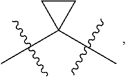 [SMILES: *C1(*)CC1]

Rₐ₃ and R_{b3} are CH₃, and Rₐ₄ and R_{b4} are

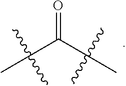 [SMILES: *C(*)=O]

In some variations of formula (12) described in the paragraphs above in which l₂ is 4, Rₐ₁ and R_{b1} are

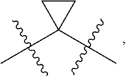 [SMILES: *C1(*)CC1]

Rₐ₂ and R_{b2} are

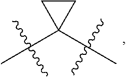 [SMILES: *C1(*)CC1]

Rₐ₃ and R_{b3} are CH₃, and Rₐ₄ and R_{b4} are

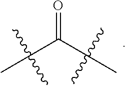 [SMILES: *C(*)=O]

In some variations of formula (12) described in the paragraphs above in which l₂ is 4, Rₐ₁ and R_{b1} are

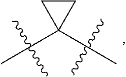 [SMILES: *C1(*)CC1]

Rₐ₂ and R_{b2} are

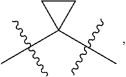 [SMILES: *C1(*)CC1]

Rₐ₃ and R_{b3} are H, and Rₐ₄ and R_{b4} are

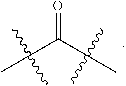 [SMILES: *C(*)=O]

In some variations of formula (12) described in the paragraphs above in which l₂ is 4, Rₐ₁ and R_{b1} are

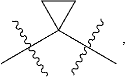 [SMILES: *C1(*)CC1]

Rₐ₂ and R_{b2} are

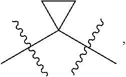 [SMILES: *C1(*)CC1]

Rₐ₃ and R_{b3} are

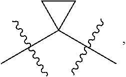 [SMILES: *C1(*)CC1]

and Rₐ₄ and R_{b4} are

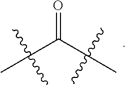 [SMILES: *C(*)=O]

In some variations of formula (12) described in the paragraphs above in which l₂ is 4, Rₐ₁ and R_{b1} are

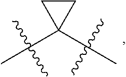 [SMILES: *C1(*)CC1]

Rₐ₂ and R_{b2} are

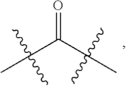 [SMILES: *C(*)=O]

Rₐ₃ and R_{b3} are H, and Rₐ₄ and R_{b4} are

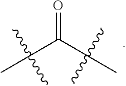 [SMILES: *C(*)=O]

In some variations of formula (12) described in the paragraphs above in which l₂ is 4, Rₐ₁ and R_{b1} are

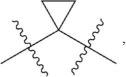 [SMILES: *C1(*)CC1]

Rₐ₂ and R_{b2} are

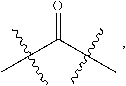 [SMILES: *C(*)=O]

Rₐ₃ and R_{b3} are CH₃, and Rₐ₄ and R_{b4} are

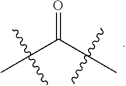 [SMILES: *C(*)=O]

In some variations of formula (12) described in the paragraphs above in which l₂ is 4, Rₐ₁ and R_{b1} are

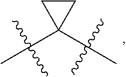 [SMILES: *C1(*)CC1]

Rₐ₂ and R_{b2} are

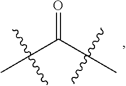 [SMILES: *C(*)=O]

Rₐ₃ and R_{b3} are CH₃, and Rₐ₄ and R_{b4} are

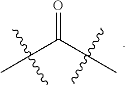 [SMILES: *C(*)=O]

In some variations of formula (12) described in the paragraphs above in which l₂ is 4, Rₐ₁ and R_{b1} are

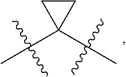 [SMILES: *C1(*)CC1]

Rₐ₂ and R_{b2} are

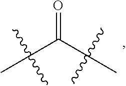 [SMILES: *C(*)=O]

Rₐ₃ and R_{b3} are H, and Rₐ₄ and R_{b4} are

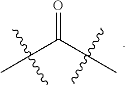 [SMILES: *C(*)=O]

In some variations of formula (12) described in the paragraphs above in which l₂ is 4, Rₐ₁ and R_{b1} are

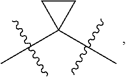 [SMILES: *C1(*)CC1]

Rₐ₂ and R_{b2} are

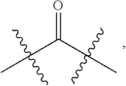 [SMILES: *C(*)=O]

Rₐ₃ and R_{b3} are

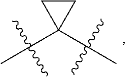 [SMILES: *C1(*)CC1]

and Rₐ₄ and R_{b4} are

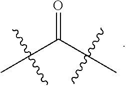 [SMILES: *C(*)=O]

In some variations of formula (12) described in the paragraphs above in which l₂ is 4, Rₐ₁ and R_{b1} are

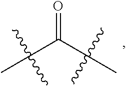 [SMILES: *C(*)=O]

Rₐ₂ and R_{b2} are

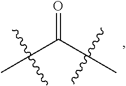 [SMILES: *C(*)=O]

Rₐ₃ and R_{b3} are H, and Rₐ₄ and R_{b4} are

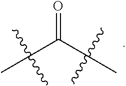 [SMILES: *C(*)=O]

In some variations of formula (12) described in the paragraphs above in which l₂ is 4, Rₐ₁ and R_{b1} are

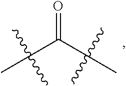 [SMILES: *C(*)=O]

Rₐ₂ and R_{b2} are

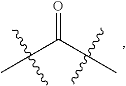 [SMILES: *C(*)=O]

Rₐ₃ and R_{b3} are CH₃, and Rₐ₄ and R_{b4} are

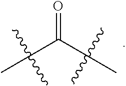 [SMILES: *C(*)=O]

In some variations of formula (12) described in the paragraphs above in which l₂ is 4, Rₐ₁ and R_{b1} are

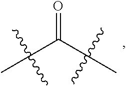 [SMILES: *C(*)=O]

Rₐ₂ and R_{b2} are

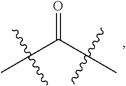 [SMILES: *C(*)=O]

Rₐ₃ and R_{b3} are CH₃, and Rₐ₄ and R_{b4} are

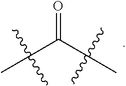 [SMILES: *C(*)=O]

In some variations of formula (12) described in the paragraphs above in which l₂ is 4, Rₐ₁ and R_{b1} are

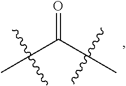 [SMILES: *C(*)=O]

Rₐ₂ and R_{b2} are

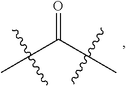 [SMILES: *C(*)=O]

Rₐ₃ and R_{b3} are H, and Rₐ₄ and R_{b4} are

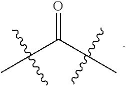 [SMILES: *C(*)=O]

In some variations of formula (12) described in the paragraphs above in which l₂ is 4, Rₐ₁ and R_{b1} are

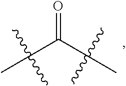 [SMILES: *C(*)=O]

Rₐ₂ and R_{b2} are

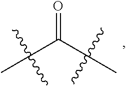 [SMILES: *C(*)=O]

Rₐ₃ and R_{b3} are

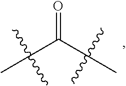 [SMILES: *C(*)=O]

and Rₐ₄ and R_{b4} are

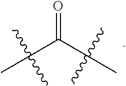 [SMILES: *C(*)=O]

In some variations of formula (12) described in the paragraphs above in which l₂ is 4, Rₐ₁ and R_{b1} are H, Rₐ₂ and R_{b2} are H, and Rₐ₃ and R_{b3} and Rₐ₄ and R_{b4} together are

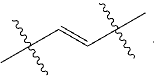 [SMILES: */C=C/*]

In some variations of formula (12) described in the paragraphs above in which l₂ is 4, Rₐ₁ and R_{b1} are H, Rₐ₂ and R_{b2} are CH₃, and Rₐ₃ and R_{b3} and Rₐ₄ and R_{b4} together are

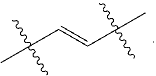 [SMILES: */C=C/*]

In some variations of formula (12) described in the paragraphs above in which l₂ is 4, Rₐ₁ and Rₐ₂ are H, R_{b1} and R_{b2} are CH₃, and Rₐ₃ and R_{b3} and Rₐ₄ and R_{b4} together are

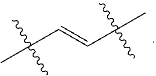 [SMILES: */C=C/*]

In some variations of formula (12) described in the paragraphs above in which l₂ is 4, Rₐ₁ and R_{b1} are H, Rₐ₂ and R_{b2} are

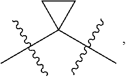 [SMILES: *C1(*)CC1]

and Rₐ₃ and R_{b3} and Rₐ₄ and R_{b4} together are

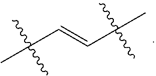 [SMILES: */C=C/*]

In some variations of formula (12) described in the paragraphs above in which l₂ is 4, Rₐ₁ and R_{b1} are

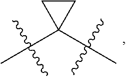 [SMILES: *C1(*)CC1]

Rₐ₂ and R_{b2} are CH₃, and Rₐ₃ and R_{b3} and Rₐ₄ and R_{b4} together are

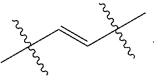 [SMILES: */C=C/*]

In some variations of formula (12) described in the paragraphs above in which l₂ is 4, Rₐ₁ is H, R_{b1} is CH₃, Rₐ₂ and R_{b2} are

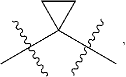 [SMILES: *C1(*)CC1]

and Rₐ₃ and R_{b3} and Rₐ₄ and R_{b4} together are

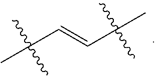 [SMILES: */C=C/*]

In some variations of formula (12) described in the paragraphs above in which l₂ is 4, Rₐ₁ and R_{b1} are H, Rₐ₂ and R_{b2} are

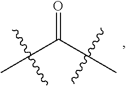 [SMILES: *C(*)=O]

and Rₐ₃ and R_{b3} and Rₐ₄ and R_{b4} together are

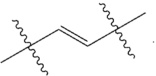 [SMILES: */C=C/*]

In some variations of formula (12) described in the paragraphs above in which l₂ is 4, Rₐ₁ and R_{b1} are

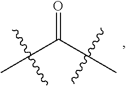 [SMILES: *C(*)=O]

Rₐ₂ and R_{b2} are CH₃, and Rₐ₃ and R_{b3} and Rₐ₄ and R_{b4} together are

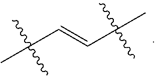 [SMILES: */C=C/*]

In some variations of formula (12) described in the paragraphs above in which l₂ is 4, Rₐ₁ is H, R_{b1} is CH₃, Rₐ₂ and R_{b2} are

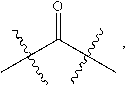 [SMILES: *C(*)=O]

and Rₐ₃ and R_{b3} and Rₐ₄ and R_{b4} together are

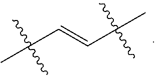 [SMILES: */C=C/*]

In some variations of formula (12) described in the paragraphs above in which l₂ is 4, Rₐ₁ and R_{b1} are

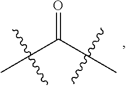 [SMILES: *C(*)=O]

Rₐ₂ and R_{b2} are

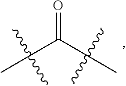 [SMILES: *C(*)=O]

and Rₐ₃ and R_{b3} and Rₐ₄ and R_{b4} together are

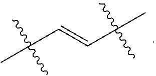 [SMILES: */C=C/*]

In some variations of formula (12) described in the paragraphs above in which l₂ is 4, Rₐ₁ and R_{b1} are

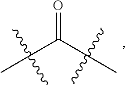 [SMILES: *C(*)=O]

Rₐ₂ and R_{b2} are

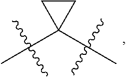 [SMILES: *C1(*)CC1]

and Rₐ₃ and R_{b3} and Rₐ₄ and R_{b4} together are

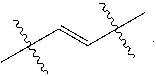 [SMILES: */C=C/*]

In some variations of formula (12) described in the paragraphs above in which l₂ is 4, Rₐ₁ and R_{b1} are

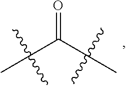 [SMILES: *C(*)=O]

Rₐ₂ and R_{b2} are

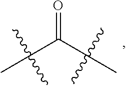 [SMILES: *C(*)=O]

and Rₐ₃ and R_{b3} and Rₐ₄ and R_{b4} together are

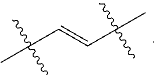 [SMILES: */C=C/*]

In some variations of formula (12) described in the paragraphs above in which l₂ is 4, Rₐ₁ and R_{b1} are

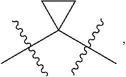 [SMILES: *C1(*)CC1]

Rₐ₂ and R_{b2} are

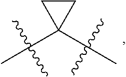 [SMILES: *C1(*)CC1]

and Rₐ₃ and R_{b3} and Rₐ₄ and R_{b4} together are

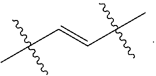 [SMILES: */C=C/*]

In some variations of formula (12) described in the paragraphs above in which l₂ is 4, Rₐ₁ and R_{b1} and Rₐ₂ and R_{b2} together are

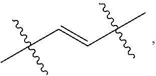 [SMILES: */C=C/*]

and Rₐ₁ and R_{b3} and Rₐ₄ and R_{b4} together are

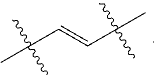 [SMILES: */C=C/*]

In some variations of formula (12) described in the paragraphs above in which o₁ is 1, Rₐ₁ and R_{b1} are both H. In some variations of formula (12) described in the paragraphs above in which o₁ is 1, Rₐ₁ is H and R_{b1} is CH₃. In some variations of formula (12) described in the paragraphs above in which o₁ is 1, Rₐ₁ and R_{b1} are both CH₃. In some variations of formula (12) described in the paragraphs above in which o₁ is 1, Rₐ₁ and R_{b1} are

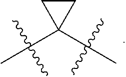 [SMILES: *C1(*)CC1]

In some variations of formula (12) described in the paragraphs above in which o₁ is 1, Rₐ₁ and R_{b1} are

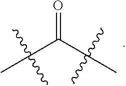 [SMILES: *C(*)=O]

In variations of formula (12) described in the paragraphs above in which o₁ is 2, the two instances of Rₐ and R_{b} are indicated as Rₐ₁ and R_{b1} and Rₐ₂ and R_{b2}, respectively. In some variations of formula (12) described in the paragraphs above in which o₁ is 2, each of Rₐ₁ and R_{b1} and Rₐ₂ and R_{b2} is H. In some variations of formula (12) described in the paragraphs above in which o₁ is 2, each of Rₐ₁ and R_{b1} and Rₐ₂ and R_{b2} is CH₃. In some variations of formula (12) described in the paragraphs above in which o₁ is 2, each of Rₐ₁ and Rₐ₂ is H and each of R_{b1} and R_{b2} is CH₃. In some variations of formula (12) described in the paragraphs above in which o₁ is 2, each of Rₐ₁, Rₐ₂, and R_{b1} is H and R_{b2} is CH₃.

In some variations of formula (12) described in the paragraphs above in which o₁ is 2, each of Rₐ₁ and R_{b1} is H and Rₐ₂ and R_{b2} are

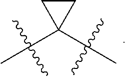 [SMILES: *C1(*)CC1]

In some variations of formula (12) described in the paragraphs above in which o₁ is 2, each of Rₐ₁ and R_{b1} is H and Rₐ₂ and R_{b2} are

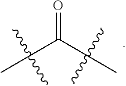 [SMILES: *C(*)=O]

In some variations of formula (12) described in the paragraphs above in which o₁ is 2, each of Rₐ₁ and R_{b1} is CH₃ and Rₐ₂ and R_{b2} are

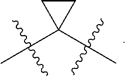 [SMILES: *C1(*)CC1]

In some variations of formula (12) described in the paragraphs above in which o₁ is 2, each of Rₐ₁ and R_{b1} is CH₃ and Rₐ₂ and R_{b2} are

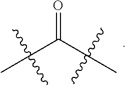 [SMILES: *C(*)=O]

In some variations of formula (12) described in the paragraphs above in which o₁ is 2, Rₐ₁ is H, R_{b1} is CH₃, and Rₐ₂ and R_{b2} are

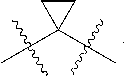 [SMILES: *C1(*)CC1]

In some variations of formula (12) described in the paragraphs above in which o₁ is 2, Rₐ₁ is H, R_{b1} is CH₃, and Rₐ₂ and R_{b2} are

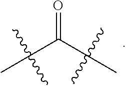 [SMILES: *C(*)=O]

In some variations of formula (12) described in the paragraphs above in which o₁ is 2, Rₐ₁ and R_{b1} and Rₐ₂ and R_{b2} together are

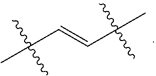 [SMILES: */C=C/*]

In some variations of formula (12) described in the paragraphs above in which o₁ is 2, Rₐ₁ and R_{b1} are

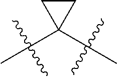 [SMILES: *C1(*)CC1]

Rₐ₂ and R_{b2} are

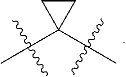 [SMILES: *C1(*)CC1]

In some variations of formula (12) described in the paragraphs above in which o₁ is 2, Rₐ₁ and R_{b1} are

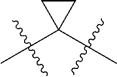 [SMILES: *C1(*)CC1]

Rₐ₂ and R_{b2} are

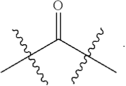 [SMILES: *C(*)=O]

In some variations of formula (12) described in the paragraphs above in which o₁ is 2, Rₐ₁ and R_{b1} are

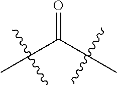 [SMILES: *C(*)=O]

Rₐ₂ and R_{b2} are

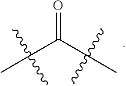 [SMILES: *C(*)=O]

In variations of formula (12) described in the paragraphs above in which o₁ is 3, the three instances of Rₐ and R_{b} are indicated as Rₐ₁ and R_{b1}; Rₐ₂ and R_{b2}; and Rₐ₃ and R_{b3}, respectively. In some variations of formula (12) described in the paragraphs above in which o₁ is 3, In some variations of formula (12) described in the paragraphs above in which o₁ is 3, each of Rₐ₁, R_{b1}, Rₐ₂, R_{b2}, Rₐ₃, and R_{b3} is H. In some variations of formula (12) described in the paragraphs above in which o₁ is 3, each of Rₐ₁, R_{b1}, Rₐ₂, R_{b2}, Rₐ₃, and R_{b3} is CH₃. In some variations of formula (12) described in the paragraphs above in which o₁ is 3, each of Rₐ₁, R_{b1}, Rₐ₂, and R_{b2}, is H and each of Rₐ₃ and R_{b3} is CH₃. In some variations of formula (12) described in the paragraphs above in which o₁ is 3, each of Rₐ₁, R_{b1}, Rₐ₂, and R_{b2}, is CH₃ and each of Rₐ₃ and R_{b3} is H. In some variations of formula (12) described in the paragraphs above in which o₁ is 3, Rₐ₁ is H and each of Rₐ₂, Rₐ₃, R_{b1}, R_{b2}, and R_{b3} is CH₃. In some variations of formula (12) described in the paragraphs above in which o₁ is 3, each of Rₐ₁ and Rₐ₂ is H and each of Rₐ₃, R_{b1}, R_{b2}, an R_{b3} is CH₃. In some variations of formula (12) described in the paragraphs above in which o₁ is 3, each of Rₐ₁, Rₐ₂, and Rₐ₃ is H and each of R_{b1}, R_{b2}, an R_{b3} is CH₃.

In some variations of formula (12) described in the paragraphs above in which o₁ is 3, each of Rₐ₁, R_{b1}, Rₐ₂, and R_{b2}, is H and Rₐ₃ and R_{b3} are

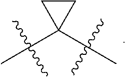 [SMILES: *C1(*)CC1]

In some variations of formula (12) described in the paragraphs above in which o₁ is 3, each of Rₐ₁, R_{b1}, Rₐ₂, and R_{b2}, is CH₃ and Rₐ₃ and R_{b3} are

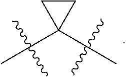 [SMILES: *C1(*)CC1]

In some variations of formula (12) described in the paragraphs above in which o₁ is 3, Rₐ₁ is H and each of Rₐ₂, R_{b1}, and R_{b2} is CH₃, and Rₐ₃ and R_{b3} are

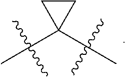 [SMILES: *C1(*)CC1]

In some variations of formula (12) described in the paragraphs above in which o₁ is 3, each of Rₐ₁ and Rₐ₂ is H and each of R_{b1} and R_{b2} is CH₃, and Rₐ₃ and R_{b3} are

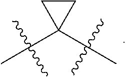 [SMILES: *C1(*)CC1]

In some variations of formula (12) described in the paragraphs above in which o₁ is 3, each of Rₐ₁, R_{b1}, Rₐ₂, and R_{b2}, is H and Rₐ₃ and R_{b3} are

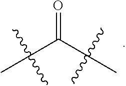 [SMILES: *C(*)=O]

In some variations of formula (12) described in the paragraphs above in which o₁ is 3, each of Rₐ₁, R_{b1}, Rₐ₂, and R_{b2}, is CH₃ and Rₐ₃ and R_{b3} are

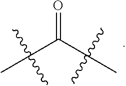 [SMILES: *C(*)=O]

In some variations of formula (12) described in the paragraphs above in which o₁ is 3, Rₐ₁ is H and each of Rₐ₂, R_{b1}, and R_{b2} is CH₃, and Rₐ₃ and R_{b3} are

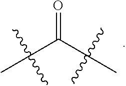 [SMILES: *C(*)=O]

In some variations of formula (12) described in the paragraphs above in which o₁ is 3, each of Rₐ₁ and Rₐ₂ is H and each of R_{b1} and R_{b2} is CH₃, and Rₐ₃ and R_{b3} are

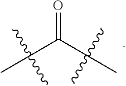 [SMILES: *C(*)=O]

In some variations of formula (12) described in the paragraphs above in which o₁ is 3, Rₐ₁ and R_{b1} are

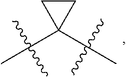 [SMILES: *C1(*)CC1]

Rₐ₂ and R_{b2} are

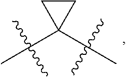 [SMILES: *C1(*)CC1]

and each of Rₐ₃ and R_{b3} is H. In some variations of formula (12) described in the paragraphs above in which o₁ is 3, Rₐ₁ and R_{b1} are

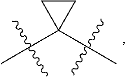 [SMILES: *C1(*)CC1]

Rₐ₂ and R_{b2} are

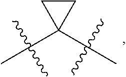 [SMILES: *C1(*)CC1]

and each of Rₐ₃ and R_{b3} is CH₃. In some variations of formula (12) described in the paragraphs above in which o₁ is 3, Rₐ₁ and R_{b1} are

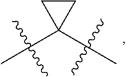 [SMILES: *C1(*)CC1]

Rₐ₂ and R_{b2} are

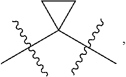 [SMILES: *C1(*)CC1]

Rₐ₃ is H, and R_{b3} is CH₃. In some variations of formula (12) described in the paragraphs above in which o₁ is 3, Rₐ₁ and R_{b1} are

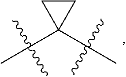 [SMILES: *C1(*)CC1]

Rₐ₂ and R_{b2} are

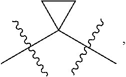 [SMILES: *C1(*)CC1]

and each of Rₐ₃ and R_{b3} is H. In some variations of formula (12) described in the paragraphs above in which o₁ is 3, Rₐ₁ and R_{b1} are

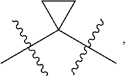 [SMILES: *C1(*)CC1]

Rₐ₂ and R_{b2} are

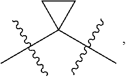 [SMILES: *C1(*)CC1]

and each of Rₐ₃ and R_{b3} is CH₃. In some variations of formula (12) described in the paragraphs above in which o₁ is 3, Rₐ₁ and R_{b1} are

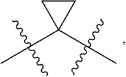 [SMILES: *C1(*)CC1]

Rₐ₂ and R_{b2} are

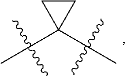 [SMILES: *C1(*)CC1]

Rₐ₃ is H, and R_{b3} is CH₃.

In some variations of formula (12) described in the paragraphs above in which o₁ is 3, Rₐ₁ and R_{b1} are

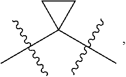 [SMILES: *C1(*)CC1]

Rₐ₂ and R_{b2} are

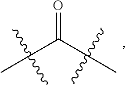 [SMILES: *C(*)=O]

and each of Rₐ₃ and R_{b3} is H. In some variations of formula (12) described in the paragraphs above in which o₁ is 3, Rₐ₁ and R_{b1} are

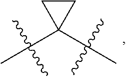 [SMILES: *C1(*)CC1]

Rₐ₂ and R_{b2} are

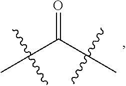 [SMILES: *C(*)=O]

and each of Rₐ₃ and R_{b3} is CH₃. In some variations of formula (12) described in the paragraphs above in which o₁ is 3, Rₐ₁ and R_{b1} are

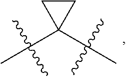 [SMILES: *C1(*)CC1]

Rₐ₂ and R_{b2} are

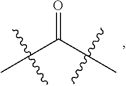 [SMILES: *C(*)=O]

Rₐ₃ is H, and R_{b3} is CH₃. In some variations of formula (12) described in the paragraphs above in which o₁ is 3, Rₐ₁ and R_{b1} are

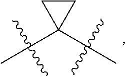 [SMILES: *C1(*)CC1]

Rₐ₂ and R_{b2} are

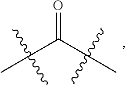 [SMILES: *C(*)=O]

and each of Rₐ₃ and R_{b3} is H. In some variations of formula (12) described in the paragraphs above in which o₁ is 3, Rₐ₁ and R_{b1} are

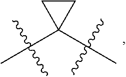 [SMILES: *C1(*)CC1]

Rₐ₂ and R_{b2} are

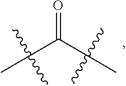 [SMILES: *C(*)=O]

and each of Rₐ₃ and R_{b3} is CH₃. In some variations of formula (12) described in the paragraphs above in which o₁ is 3, Rₐ₁ and R_{b1} are

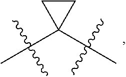 [SMILES: *C1(*)CC1]

Rₐ₂ and R_{b2} are

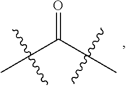 [SMILES: *C(*)=O]

Rₐ₃ is H, and R_{b3} is CH₃.

In some variations of formula (12) described in the paragraphs above in which o₁ is 3, Rₐ₁ and R_{b1} are

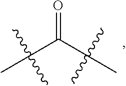 [SMILES: *C(*)=O]

Rₐ₂ and R_{b2} are

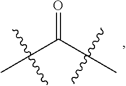 [SMILES: *C(*)=O]

and each of Rₐ₃ and R_{b3} is H. In some variations of formula (12) described in the paragraphs above in which o₁ is 3, Rₐ₁ and R_{b1} are

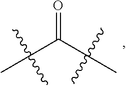 [SMILES: *C(*)=O]

Rₐ₂ and R_{b2} are

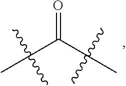 [SMILES: *C(*)=O]

and each of Rₐ₃ and R_{b3} is CH₃. In some variations of formula (12) described in the paragraphs above in which o₁ is 3, Rₐ₁ and R_{b1} are

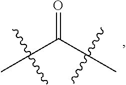 [SMILES: *C(*)=O]

Rₐ₂ and R_{b2} are

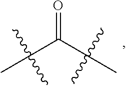 [SMILES: *C(*)=O]

Rₐ₃ is H, and R_{b3} is CH₃. In some variations of formula (12) described in the paragraphs above in which o₁ is 3, Rₐ₁ and R_{b1} are

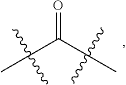 [SMILES: *C(*)=O]

Rₐ₂ and R_{b2} are

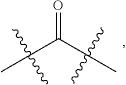 [SMILES: *C(*)=O]

and each of Rₐ₃ and R_{b3} is H. In some variations of formula (12) described in the paragraphs above in which o₁ is 3, Rₐ₁ and R_{b1} are

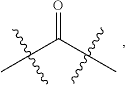 [SMILES: *C(*)=O]

Rₐ₂ and R_{b2} are

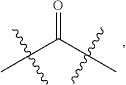 [SMILES: *C(*)=O]

and each of Rₐ₃ and R_{b3} is CH₃. In some variations of formula (12) described in the paragraphs above in which o₁ is 3, Rₐ₁ and R_{b1} are

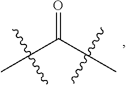 [SMILES: *C(*)=O]

Rₐ₂ and R_{b2} are

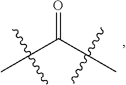 [SMILES: *C(*)=O]

Rₐ₃ is H, and R_{b3} is CH₃.

In some variations of formula (12) described in the paragraphs above in which o₁ is 3, Rₐ₁ and R_{b1} and Rₐ₂ and R_{b2} together are

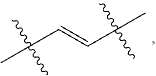 [SMILES: */C=C/*]

and each of Rₐ₃ and R_{b3} is H. In some variations of formula (12) described in the paragraphs above in which o₁ is 3, Rₐ₁ and R_{b1} and Rₐ₂ and R_{b2} together are

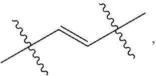 [SMILES: */C=C/*]

and each of Rₐ₃ and R_{b3} is CH₃. In some variations of formula (12) described in the paragraphs above in which o₁ is 3, Rₐ₁ and R_{b1} and Rₐ₂ and R_{b2} together are

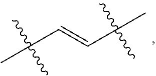 [SMILES: */C=C/*]

Rₐ₃ is H, and R_{b3} is CH₃. In some variations of formula (12) described in the paragraphs above in which o₁ is 3, Rₐ₁ and R_{b1} and Rₐ₂ and R_{b2} together are

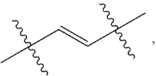 [SMILES: */C=C/*]

and Rₐ₃ and R_{b3} are

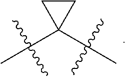 [SMILES: *C1(*)CC1]

In some variations of formula (12) described in the paragraphs above in which o₁ is 3, Rₐ₁ and R_{b1} and Rₐ₂ and R_{b2} together are

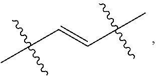 [SMILES: */C=C/*]

and Rₐ₃ and R_{b3} are

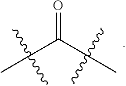 [SMILES: *C(*)=O]

In variations of formula (12) described in the paragraphs above in which o₁ is 4, the three instances of Rₐ and R_{b} are indicated as Rₐ₁ and R_{b1}; Rₐ₂ and R_{b2}; Rₐ₃ and R_{b3}; and Rₐ₄ and R_{b4}, respectively. In some variations of formula (12) described in the paragraphs above in which o₁ is 4, In some variations of formula (12) described in the paragraphs above in which o₁ is 4, each of Rₐ₁, R_{b1}, Rₐ₂, R_{b2}, Rₐ₃, R_{b3}, Rₐ₄, and R_{b4} is H. In some variations of formula (12) described in the paragraphs above in which o₁ is 4, each of Rₐ₁, R_{b1}, Rₐ₂, R_{b2}, Rₐ₃, R_{b3}, Rₐ₄, and R_{b4} is CH₃. In some variations of formula (12) described in the paragraphs above in which o₁ is 4, each of Rₐ₁, R_{b1}, Rₐ₂, R_{b2}, Rₐ₃, and R_{b3} is H and each of Rₐ₄ and R_{b4} is CH₃. In some variations of formula (12) described in the paragraphs above in which o₁ is 4, each of Rₐ₁, R_{b1}, Rₐ₂, and R_{b2} is H and each of Rₐ₃, R_{b3}, Rₐ₄, and R_{b4} is CH₃. In some variations of formula (12) described in the paragraphs above in which o₁ is 4, each of Rₐ₁, R_{b1}, Rₐ₂, R_{b2}, Rₐ₃, and R_{b3} is CH₃ and each of Rₐ₄ and R_{b4} is H.

In some variations of formula (12) described in the paragraphs above in which o₁ is 4, each of Rₐ₁, Rₐ₂, and Rₐ₃ is H and each of R_{b1}, R_{b2}, and R_{b3} is CH₃. In some variations of formula (12) described in the paragraphs above in which o₁ is 4, Rₐ₁ is H, R_{b1} is CH₃, and each of Rₐ₂, R_{b2}, Rₐ₃, and R_{b3} is CH₃. In some variations of formula (12) described in the paragraphs above in which o₁ is 4, Rₐ₁ is H, R_{b1} is CH₃, and each of Rₐ₂, R_{b2}, Rₐ₃, and R_{b3} is H. In some variations of formula (12) described in the paragraphs above in which o₁ is 4, each of Rₐ₁ and Rₐ₂ is H, each of R_{b1} and R_{b2} is CH₃, and each of Rₐ₃ and R_{b3} is H. In some variations of formula (12) described in the paragraphs above in which o₁ is 4, each of Rₐ₁ and Rₐ₂ is H, each of R_{b1} and R_{b2} is CH₃, and each of Rₐ₃ and R_{b3} is CH₃.

In some variations of formula (12) described in the paragraphs above in which o₁ is 4, Rₐ₁ and R_{b1} are H, Rₐ₂ and R_{b2} are H, Rₐ₃ and R_{b3} are H, and Rₐ₄ and R_{b4} are

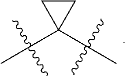 [SMILES: *C1(*)CC1]

In some variations of formula (12) described in the paragraphs above in which o₁ is 4, Rₐ₁ and R_{b1} are H, Rₐ₂ and R_{b2} are H, Rₐ₃ and R_{b3} are CH₃, and Rₐ₄ and R_{b4} are

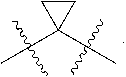 [SMILES: *C1(*)CC1]

In some variations of formula (12) described in the paragraphs above in which o₁ is 4, Rₐ₁ and R_{b1} are H, Rₐ₂ and R_{b2} are CH₃, Rₐ₃ and R_{b3} are CH₃, and Rₐ₄ and R_{b4} are

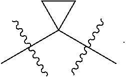 [SMILES: *C1(*)CC1]

In some variations of formula (12) described in the paragraphs above in which o₁ is 4, Rₐ₁ and R_{b1} are CH₃, Rₐ₂ and R_{b2} are CH₃, Rₐ₃ and R_{b3} are CH₃, and Rₐ₄ and R_{b4} are

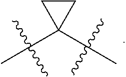 [SMILES: *C1(*)CC1]

In some variations of formula (12) described in the paragraphs above in which o₁ is 4, Rₐ₁ and R_{b1} are H, Rₐ₂ and R_{b2} are H, Rₐ₃ and R_{b3} are

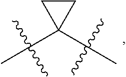 [SMILES: *C1(*)CC1]

and Rₐ₄ and R_{b4} are

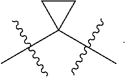 [SMILES: *C1(*)CC1]

In some variations of formula (12) described in the paragraphs above in which o₁ is 4, Rₐ₁ and R_{b1} are H, Rₐ₂ and R_{b2} are CH₃, Rₐ₃ and R_{b3} are

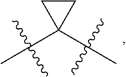 [SMILES: *C1(*)CC1]

and Rₐ₄ and R_{b4} are

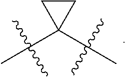 [SMILES: *C1(*)CC1]

In some variations of formula (12) described in the paragraphs above in which o₁ is 4, Rₐ₁ and R_{b1} are CH₃, Rₐ₂ and R_{b2} are CH₃, Rₐ₃ and R_{b3} are

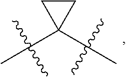 [SMILES: *C1(*)CC1]

and Rₐ₄ and R_{b4} are

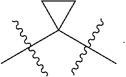 [SMILES: *C1(*)CC1]

In some variations of formula (12) described in the paragraphs above in which o₁ is 4, Rₐ₁ is H, R_{b1} is CH₃, Rₐ₂ and R_{b2} are CH₃, Rₐ₃ and R_{b3} are

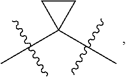 [SMILES: *C1(*)CC1]

and Rₐ₄ and R_{b4} are

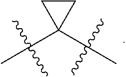 [SMILES: *C1(*)CC1]

In some variations of formula (12) described in the paragraphs above in which o₁ is 4, Rₐ₁ is H, R_{b1} is CH₃, Rₐ₂ is H, R_{b2} is CH₃, Rₐ₃ and R_{b3} are

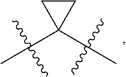 [SMILES: *C1(*)CC1]

and Rₐ₄ and R_{b4} are

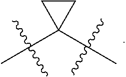 [SMILES: *C1(*)CC1]

In some variations of formula (12) described in the paragraphs above in which o₁ is 4, Rₐ₁ and R_{b1} are H, Rₐ₂ and R_{b2} are

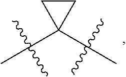 [SMILES: *C1(*)CC1]

Rₐ₃ and R_{b3} are

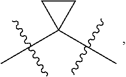 [SMILES: *C1(*)CC1]

and Rₐ₄ and R_{b4} are

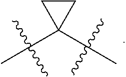 [SMILES: *C1(*)CC1]

In some variations of formula (12) described in the paragraphs above in which o₁ is 4, Rₐ₁ and R_{b1} are CH₃, Rₐ₂ and R_{b2} are

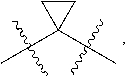 [SMILES: *C1(*)CC1]

Rₐ₃ and R_{b3} are

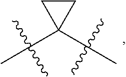 [SMILES: *C1(*)CC1]

and Rₐ₄ and R_{b4} are

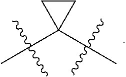 [SMILES: *C1(*)CC1]

In some variations of formula (12) described in the paragraphs above in which o₁ is 4, Rₐ₁ is H, R_{b1} is CH₃, Rₐ₂ and R_{b2} are

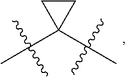 [SMILES: *C1(*)CC1]

Rₐ₃ and R_{b3} are

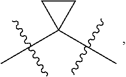 [SMILES: *C1(*)CC1]

and Rₐ₄ and R_{b4} are

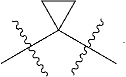 [SMILES: *C1(*)CC1]

In some variations of formula (12) described in the paragraphs above in which o₁ is 4, Rₐ₁ and R_{b1} are

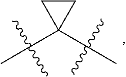 [SMILES: *C1(*)CC1]

Rₐ₂ and R_{b2} are

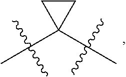 [SMILES: *C1(*)CC1]

Rₐ₃ and R_{b3} are

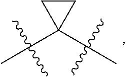 [SMILES: *C1(*)CC1]

and Rₐ₄ and R_{b4} are

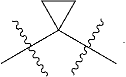 [SMILES: *C1(*)CC1]

In some variations of formula (12) described in the paragraphs above in which o₁ is 4, Rₐ₁ and R_{b1} are H, Rₐ₂ and R_{b2} are H, Rₐ₃ and R_{b3} are H, and Rₐ₄ and R_{b4} are

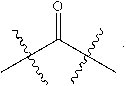 [SMILES: *C(*)=O]

In some variations of formula (12) described in the paragraphs above in which o₁ is 4, Rₐ₁ and R_{b1} are H, Rₐ₂ and R_{b2} are H, Rₐ₃ and R_{b3} are CH₃, and Rₐ₄ and R_{b4} are

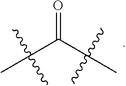 [SMILES: *C(*)=O]

In some variations of formula (12) described in the paragraphs above in which o₁ is 4, Rₐ₁ and R_{b1} are H, Rₐ₂ and R_{b2} are CH₃, Rₐ₃ and R_{b3} are CH₃, and Rₐ₄ and R_{b4} are

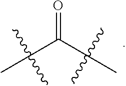 [SMILES: *C(*)=O]

In some variations of formula (12) described in the paragraphs above in which o₁ is 4, Rₐ₁ and R_{b1} are CH₃, Rₐ₂ and R_{b2} are CH₃, Rₐ₃ and R_{b3} are CH₃, and Rₐ₄ and R_{b4} are

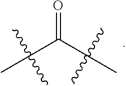 [SMILES: *C(*)=O]

In some variations of formula (12) described in the paragraphs above in which o₁ is 4, Rₐ₁ and R_{b1} are H, Rₐ₂ and R_{b2} are H, Rₐ₃ and R_{b3} are

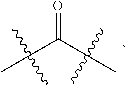 [SMILES: *C(*)=O]

and Rₐ₄ and R_{b4} are v. In some variations of formula (12) described in the paragraphs above in which o₁ is 4, Rₐ₁ and R_{b1} are H, Rₐ₂ and R_{b2} are CH₃, Rₐ₃ and R_{b3} are

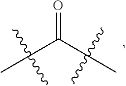 [SMILES: *C(*)=O]

and Rₐ₄ and R_{b4} are

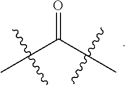 [SMILES: *C(*)=O]

In some variations of formula (12) described in the paragraphs above in which o₁ is 4, Rₐ₁ and R_{b1} are CH₃, Rₐ₂ and R_{b2} are CH₃, Rₐ₃ and R_{b3} are

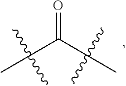 [SMILES: *C(*)=O]

and Rₐ₄ and R_{b4} are

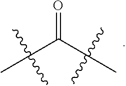 [SMILES: *C(*)=O]

In some variations of formula (12) described in the paragraphs above in which o₁ is 4, Rₐ₁ is H, R_{b1} is CH₃, Rₐ₂ and R_{b2} are CH₃, Rₐ₃ and R_{b3} are

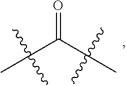 [SMILES: *C(*)=O]

and Rₐ₄ and R_{b4} are

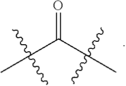 [SMILES: *C(*)=O]

In some variations of formula (12) described in the paragraphs above in which o₁ is 4, Rₐ₁ is H, R_{b1} is CH₃, Rₐ₂ is H, R_{b2} is CH₃, Rₐ₃ and R_{b3} are

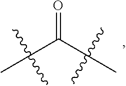 [SMILES: *C(*)=O]

and Rₐ₄ and R_{b4} are

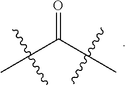 [SMILES: *C(*)=O]

In some variations of formula (12) described in the paragraphs above in which o₁ is 4, Rₐ₁ and R_{b1} are H, Rₐ₂ and R_{b2} are

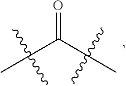 [SMILES: *C(*)=O]

Rₐ₃ and R_{b3} are

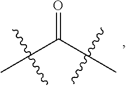 [SMILES: *C(*)=O]

and Rₐ₄ and R_{b4} are

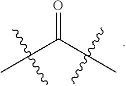 [SMILES: *C(*)=O]

In some variations of formula (12) described in the paragraphs above in which o₁ is 4, Rₐ₁ and R_{b1} are CH₃, Rₐ₂ and R_{b2} are

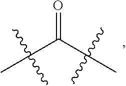 [SMILES: *C(*)=O]

Rₐ₃ and R_{b3} are

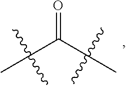 [SMILES: *C(*)=O]

and Rₐ₄ and R_{b4} are

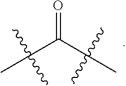 [SMILES: *C(*)=O]

In some variations of formula (12) described in the paragraphs above in which o₁ is 4, Rₐ₁ is H, R_{b1} is CH₃, Rₐ₂ and R_{b2} are

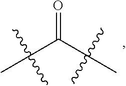 [SMILES: *C(*)=O]

Rₐ₃ and R_{b3} are

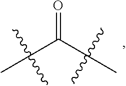 [SMILES: *C(*)=O]

and Rₐ₄ and R_{b4} are

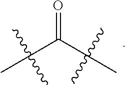 [SMILES: *C(*)=O]

In some variations of formula (12) described in the paragraphs above in which o₁ is 4, Rₐ₁ and R_{b1} are

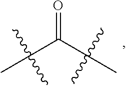 [SMILES: *C(*)=O]

Rₐ₂ and R_{b2} are

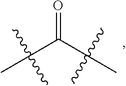 [SMILES: *C(*)=O]

Rₐ₃ and R_{b3} are

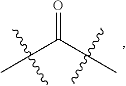 [SMILES: *C(*)=O]

and Rₐ₄ and R_{b4} are

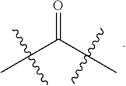 [SMILES: *C(*)=O]

In some variations of formula (12) described in the paragraphs above in which o₁ is 4, Rₐ₁ and R_{b1} are

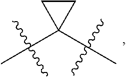 [SMILES: *C1(*)CC1]

Rₐ₂ and R_{b2} are H, Rₐ₃ and R_{b3} are H, and Rₐ₄ and R_{b4} are

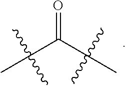 [SMILES: *C(*)=O]

In some variations of formula (12) described in the paragraphs above in which o₁ is 4, Rₐ₁ and R_{b1} are

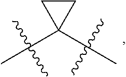 [SMILES: *C1(*)CC1]

Rₐ₂ and R_{b2} are CH₃, Rₐ₃ and R_{b3} are CH₃, and Rₐ₄ and R_{b4} are

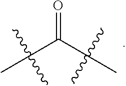 [SMILES: *C(*)=O]

In some variations of formula (12) described in the paragraphs above in which o₁ is 4, Rₐ₁ and R_{b1} are

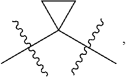 [SMILES: *C1(*)CC1]

Rₐ₂ and R_{b2} are H, Rₐ₃ and R_{b3} are CH₃, and Rₐ₄ and R_{b4} are

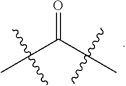 [SMILES: *C(*)=O]

In some variations of formula (12) described in the paragraphs above in which o₁ is 4, Rₐ₁ and R_{b1} are

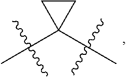 [SMILES: *C1(*)CC1]

Rₐ₂ and R_{b2} are

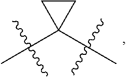 [SMILES: *C1(*)CC1]

Rₐ₃ and R_{b3} are H, and Rₐ₄ and R_{b4} are

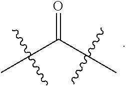 [SMILES: *C(*)=O]

In some variations of formula (12) described in the paragraphs above in which o₁ is 4, Rₐ₁ and R_{b1} are

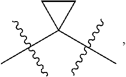 [SMILES: *C1(*)CC1]

Rₐ₂ and R_{b2} are

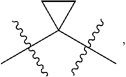 [SMILES: *C1(*)CC1]

Rₐ₃ and R_{b3} are CH₃, and Rₐ₄ and R_{b4} are

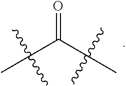 [SMILES: *C(*)=O]

In some variations of formula (12) described in the paragraphs above in which o₁ is 4, Rₐ₁ and R_{b1} are

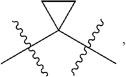 [SMILES: *C1(*)CC1]

Rₐ₂ and R_{b2} are

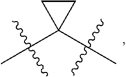 [SMILES: *C1(*)CC1]

Rₐ₃ and R_{b3} are CH₃, and Rₐ₄ and R_{b4} are

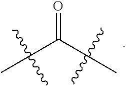 [SMILES: *C(*)=O]

In some variations of formula (12) described in the paragraphs above in which o₁ is 4, Rₐ₁ and R_{b1} are

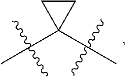 [SMILES: *C1(*)CC1]

Rₐ₂ and R_{b2} are

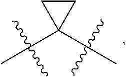 [SMILES: *C1(*)CC1]

Rₐ₃ and R_{b3} are H, and Rₐ₄ and R_{b4} are

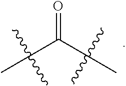 [SMILES: *C(*)=O]

In some variations of formula (12) described in the paragraphs above in which o₁ is 4, Rₐ₁ and R_{b1} are

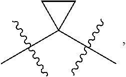 [SMILES: *C1(*)CC1]

Rₐ₂ and R_{b2} are

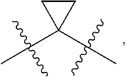 [SMILES: *C1(*)CC1]

Rₐ₃ and R_{b3} are

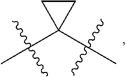 [SMILES: *C1(*)CC1]

and Rₐ₄ and R_{b4} are

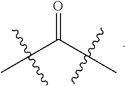 [SMILES: *C(*)=O]

In some variations of formula (12) described in the paragraphs above in which o₁ is 4, Rₐ₁ and R_{b1} are

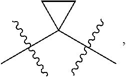 [SMILES: *C1(*)CC1]

Rₐ₂ and R_{b2} are

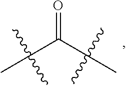 [SMILES: *C(*)=O]

Rₐ₃ and R_{b3} are H, and Rₐ₄ and R_{b4} are

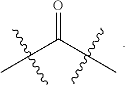 [SMILES: *C(*)=O]

In some variations of formula (12) described in the paragraphs above in which o₁ is 4, Rₐ₁ and R_{b1} are

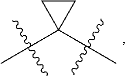 [SMILES: *C1(*)CC1]

Rₐ₂ and R_{b2} are

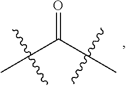 [SMILES: *C(*)=O]

Rₐ₃ and R_{b3} are CH₃, and Rₐ₄ and R_{b4} are

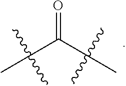 [SMILES: *C(*)=O]

In some variations of formula (12) described in the paragraphs above in which o₁ is 4, Rₐ₁ and R_{b1} are

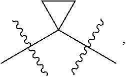 [SMILES: *C1(*)CC1]

Rₐ₂ and R_{b2} are

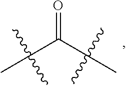 [SMILES: *C(*)=O]

Rₐ₃ and R_{b3} are CH₃, and Rₐ₄ and R_{b4} are

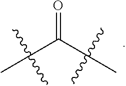 [SMILES: *C(*)=O]

In some variations of formula (12) described in the paragraphs above in which o₁ is 4, Rₐ₁ and R_{b1} are

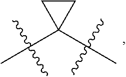 [SMILES: *C1(*)CC1]

Rₐ₂ and R_{b2} are

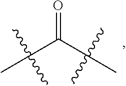 [SMILES: *C(*)=O]

Rₐ₃ and R_{b3} are H, and Rₐ₄ and R_{b4} are

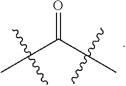 [SMILES: *C(*)=O]

In some variations of formula (12) described in the paragraphs above in which o₁ is 4, Rₐ₁ and R_{b1} are

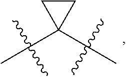 [SMILES: *C1(*)CC1]

Rₐ₂ and R_{b2} are

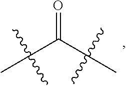 [SMILES: *C(*)=O]

Rₐ₃ and R_{b3} are

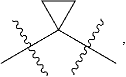 [SMILES: *C1(*)CC1]

and Rₐ₄ and R_{b4} are

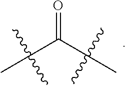 [SMILES: *C(*)=O]

In some variations of formula (12) described in the paragraphs above in which o₁ is 4, Rₐ₁ and R_{b1} are

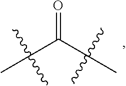 [SMILES: *C(*)=O]

Rₐ₂ and R_{b2} are

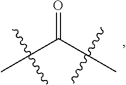 [SMILES: *C(*)=O]

Rₐ₃ and R_{b3} are H, and Rₐ₄ and R_{b4} are

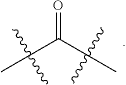 [SMILES: *C(*)=O]

In some variations of formula (12) described in the paragraphs above in which o₁ is 4, Rₐ₁ and R_{b1} are

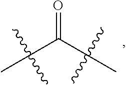 [SMILES: *C(*)=O]

Rₐ₂ and R_{b2} are

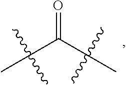 [SMILES: *C(*)=O]

Rₐ₃ and R_{b3} are CH₃, and Rₐ₄ and R_{b4} are

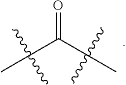 [SMILES: *C(*)=O]

In some variations of formula (12) described in the paragraphs above in which o₁ is 4, Rₐ₁ and R_{b1} are

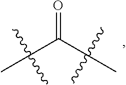 [SMILES: *C(*)=O]

Rₐ₂ and R_{b2} are

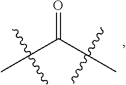 [SMILES: *C(*)=O]

Rₐ₃ and R_{b3} are CH₃, and Rₐ₄ and R_{b4} are

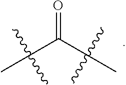 [SMILES: *C(*)=O]

In some variations of formula (12) described in the paragraphs above in which o₁ is 4, Rₐ₁ and R_{b1} are

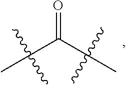 [SMILES: *C(*)=O]

Rₐ₂ and R_{b2} are

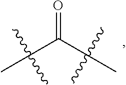 [SMILES: *C(*)=O]

Rₐ₃ and R_{b3} are H, and Rₐ₄ and R_{b4} are

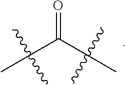 [SMILES: *C(*)=O]

In some variations of formula (12) described in the paragraphs above in which o₁ is 4, Rₐ₁ and R_{b1} are

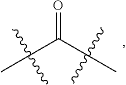 [SMILES: *C(*)=O]

Rₐ₂ and R_{b2} are

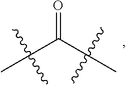 [SMILES: *C(*)=O]

Rₐ₃ and R_{b3} are

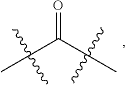 [SMILES: *C(*)=O]

and Rₐ₄ and R_{b4} are

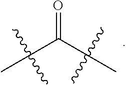 [SMILES: *C(*)=O]

In some variations of formula (12) described in the paragraphs above in which o₁ is 4, Rₐ₁ and R_{b1} are H, Rₐ₂ and R_{b2} are H, and Rₐ₃ and R_{b3} and Rₐ₄ and R_{b4} together are

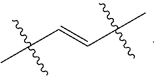 [SMILES: */C=C/*]

In some variations of formula (12) described in the paragraphs above in which o₁ is 4, Rₐ₁ and R_{b1} are H, Rₐ₂ and R_{b2} are CH₃, and Rₐ₃ and R_{b3} and Rₐ₄ and R_{b4} together are

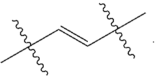 [SMILES: */C=C/*]

In some variations of formula (12) described in the paragraphs above in which o₁ is 4, Rₐ₁ and Rₐ₂ are H, R_{b1} and R_{b2} are CH₃, and Rₐ₃ and R_{b3} and Rₐ₄ and R_{b4} together are

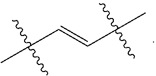 [SMILES: */C=C/*]

In some variations of formula (12) described in the paragraphs above in which o₁ is 4, Rₐ₁ and R_{b1} are H, Rₐ₂ and R_{b2} are

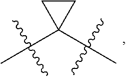 [SMILES: *C1(*)CC1]

and Rₐ₃ and R_{b3} and Rₐ₄ and R_{b4} together are

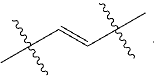 [SMILES: */C=C/*]

In some variations of formula (12) described in the paragraphs above in which o₁ is 4, Rₐ₁ and R_{b1} are

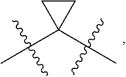 [SMILES: *C1(*)CC1]

Rₐ₂ and R_{b2} are CH₃, and Rₐ₃ and R_{b3} and Rₐ₄ and R_{b4} together are

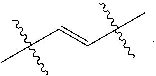 [SMILES: */C=C/*]

In some variations of formula (12) described in the paragraphs above in which o₁ is 4, Rₐ₁ is H, R_{b1} is CH₃, Rₐ₂ and R_{b2} are

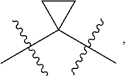 [SMILES: *C1(*)CC1]

and Rₐ₃ and R_{b3} and Rₐ₄ and R_{b4} together are

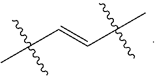 [SMILES: */C=C/*]

In some variations of formula (12) described in the paragraphs above in which o₁ is 4, Rₐ₁ and R_{b1} are H, Rₐ₂ and R_{b2} are

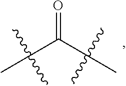 [SMILES: *C(*)=O]

and Rₐ₃ and R_{b3} and Rₐ₄ and R_{b4} together are

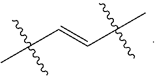 [SMILES: */C=C/*]

In some variations of formula (12) described in the paragraphs above in which o₁ is 4, Rₐ₁ and R_{b1} are

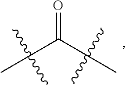 [SMILES: *C(*)=O]

Rₐ₂ and R_{b2} are CH₃, and Rₐ₃ and R_{b3} and Rₐ₄ and R_{b4} together are

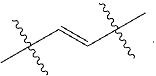 [SMILES: */C=C/*]

In some variations of formula (12) described in the paragraphs above in which o₁ is 4, Rₐ₁ is H, R_{b1} is CH₃, Rₐ₂ and R_{b2} are

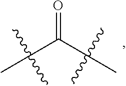 [SMILES: *C(*)=O]

and Rₐ₃ and R_{b3} and Rₐ₄ and R_{b4} together are

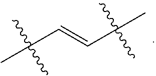 [SMILES: */C=C/*]

In some variations of formula (12) described in the paragraphs above in which o₁ is 4, Rₐ₁ and R_{b1} are

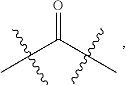 [SMILES: *C(*)=O]

Rₐ₂ and R_{b2} are

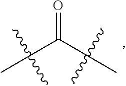 [SMILES: *C(*)=O]

and Rₐ₃ and R_{b3} and Rₐ₄ and R_{b4} together are

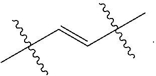 [SMILES: */C=C/*]

In some variations of formula (12) described in the paragraphs above in which o₁ is 4, Rₐ₁ and R_{b1} are

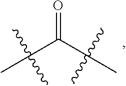 [SMILES: *C(*)=O]

Rₐ₂ and R_{b2} are

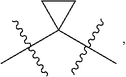 [SMILES: *C1(*)CC1]

and Rₐ₃ and R_{b3} and Rₐ₄ and R_{b4} together are

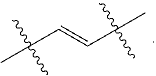 [SMILES: */C=C/*]

In some variations of formula (12) described in the paragraphs above in which o₁ is 4, Rₐ₁ and R_{b1} are

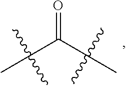 [SMILES: *C(*)=O]

Rₐ₂ and R_{b2} are

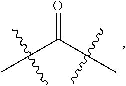 [SMILES: *C(*)=O]

and Rₐ₃ and R_{b3} and Rₐ₄ and R_{b4} together are

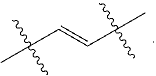 [SMILES: */C=C/*]

In some variations of formula (12) described in the paragraphs above in which o₁ is 4, Rₐ₁ and R_{b1} are

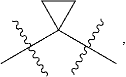 [SMILES: *C1(*)CC1]

Rₐ₂ and R_{b2} are

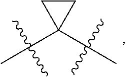 [SMILES: *C1(*)CC1]

and Rₐ₃ and R_{b3} and Rₐ₄ and R_{b4} together are

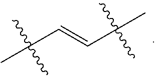 [SMILES: */C=C/*]

In some variations of formula (12) described in the paragraphs above in which o₁ is 4, Rₐ₁ and R_{b1} and Rₐ₂ and R_{b2} together are

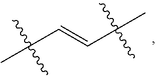 [SMILES: */C=C/*]

and Rₐ₃ and R_{b3} and Rₐ₄ and R_{b4} together are

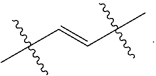 [SMILES: */C=C/*]

In some variations of formula (12) described in the paragraphs above in which o₂ is 1, Rₐ₁ and R_{b1} are both H. In some variations of formula (12) described in the paragraphs above in which o₂ is 1, Rₐ₁ is H and R_{b1} is CH₃. In some variations of formula (12) described in the paragraphs above in which o₂ is 1, Rₐ₁ and R_{b1} are both CH₃. In some variations of formula (12) described in the paragraphs above in which o₂ is 1, Rₐ₁ and R_{b1} are

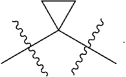 [SMILES: *C1(*)CC1]

In some variations of formula (12) described in the paragraphs above in which o₂ is 1, Rₐ₁ and R_{b1} are

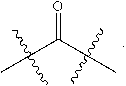 [SMILES: *C(*)=O]

In variations of formula (12) described in the paragraphs above in which o₂ is 2, the two instances of Rₐ and R_{b} are indicated as Rₐ₁ and R_{b1} and Rₐ₂ and R_{b2}, respectively. In some variations of formula (12) described in the paragraphs above in which o₂ is 2, each of Rₐ₁ and R_{b1} and Rₐ₂ and R_{b2} is H. In some variations of formula (12) described in the paragraphs above in which o₂ is 2, each of Rₐ₁ and R_{b1} and Rₐ₂ and R_{b2} is CH₃. In some variations of formula (12) described in the paragraphs above in which o₂ is 2, each of Rₐ₁ and Rₐ₂ is H and each of R_{b1} and R_{b2} is CH₃. In some variations of formula (12) described in the paragraphs above in which o₂ is 2, each of Rₐ₁, Rₐₑ, and R_{b1} is H and R_{b2} is CH₃.

In some variations of formula (12) described in the paragraphs above in which o₂ is 2, each of Rₐ₁ and R_{b1} is H and Rₐ₂ and R_{b2} are

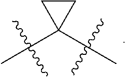 [SMILES: *C1(*)CC1]

In some variations of formula (12) described in the paragraphs above in which o₂ is 2, each of Rₐ₁ and R_{b1} is H and Rₐ₂ and R_{b2} are

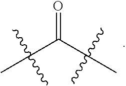 [SMILES: *C(*)=O]

In some variations of formula (12) described in the paragraphs above in which o₂ is 2, each of Rₐ₁ and R_{b1} is CH₃ and Rₐ₂ and R_{b2} are

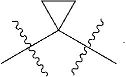 [SMILES: *C1(*)CC1]

In some variations of formula (12) described in the paragraphs above in which o₂ is 2, each of Rₐ₁ and R_{b1} is CH₃ and Rₐ₂ and R_{b2} are

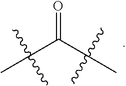 [SMILES: *C(*)=O]

In some variations of formula (12) described in the paragraphs above in which o₂ is 2, Rₐ₁ is H, R_{b1} is CH₃, and Rₐ₂ and R_{b2} are

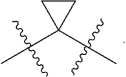 [SMILES: *C1(*)CC1]

In some variations of formula (12) described in the paragraphs above in which o₂ is 2, Rₐ₁ is H, R_{b1} is CH₃, and Rₐ₂ and R_{b2} are

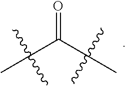 [SMILES: *C(*)=O]

In some variations of formula (12) described in the paragraphs above in which o₂ is 2, Rₐ₁ and R_{b1} and Rₐ₂ and R_{b2} together are

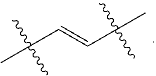 [SMILES: */C=C/*]

In some variations of formula (12) described in the paragraphs above in which o₂ is 2, Rₐ₁ and R_{b1} are

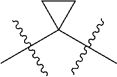 [SMILES: *C1(*)CC1]

Rₐ₂ and R_{b2} are

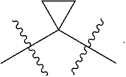 [SMILES: *C1(*)CC1]

In some variations of formula (12) described in the paragraphs above in which o₂ is 2, Rₐ₁ and R_{b1} are

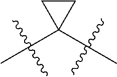 [SMILES: *C1(*)CC1]

Rₐ₂ and R_{b2} are

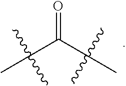 [SMILES: *C(*)=O]

In some variations of formula (12) described in the paragraphs above in which o₂ is 2, Rₐ₁ and R_{b1} are

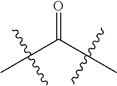 [SMILES: *C(*)=O]

Rₐ₂ and R_{b2} are

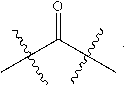 [SMILES: *C(*)=O]

In variations of formula (12) described in the paragraphs above in which o₂ is 3, the three instances of Rₐ and R_{b} are indicated as Rₐ₁ and R_{b1}; Rₐ₂ and R_{b2}; and Rₐ₃ and R_{b3}, respectively. In some variations of formula (12) described in the paragraphs above in which o₂ is 3, In some variations of formula (12) described in the paragraphs above in which o₂ is 3, each of Rₐ₁, R_{b1}, Rₐ₂, R_{b2}, Rₐ₃, and R_{b3} is H. In some variations of formula (12) described in the paragraphs above in which o₂ is 3, each of Rₐ₁, R_{b1}, Rₐ₂, R_{b2}, Rₐ₃, and R_{b3} is CH₃. In some variations of formula (12) described in the paragraphs above in which o₂ is 3, each of Rₐ₁, R_{b1}, Rₐ₂, an R_{b2}, is H and each of Rₐ₃ and R_{b3} is CH₃. In some variations of formula (12) described in the paragraphs above in which o₂ is 3, each of Rₐ₁, R_{b1}, Rₐ₂, and R_{b2}, is CH₃ and each of Rₐ₃ and R_{b3} is H. In some variations of formula (12) described in the paragraphs above in which o₂ is 3, Rₐ₁ is H and each of Rₐ₂, Rₐ₃, R_{b1}, R_{b2}, and R_{b3} is CH₃. In some variations of formula (12) described in the paragraphs above in which o₂ is 3, each of Rₐ₁ and Rₐ₂ is H and each of Rₐ₃, R_{b1}, R_{b2}, and R_{b3} is CH₃. In some variations of formula (12) described in the paragraphs above in which o₂ is 3, each of Rₐ₁, Rₐ₂, and Rₐ₃ is H and each of R_{b1}, R_{b2}, an R_{b3} is CH₃.

In some variations of formula (12) described in the paragraphs above in which o₂ is 3, each of Rₐ₁, R_{b1}, Rₐ₂, and R_{b2}, is H and Rₐ₃ and R_{b3} are

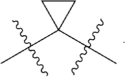 [SMILES: *C1(*)CC1]

In some variations of formula (12) described in the paragraphs above in which o₂ is 3, each of Rₐ₁, R_{b1}, Rₐ₂, and R_{b2}, is CH₃ and Rₐ₃ and R_{b3} are

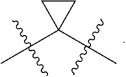 [SMILES: *C1(*)CC1]

In some variations of formula (12) described in the paragraphs above in which o₂ is 3, Rₐ₁ is H and each of Rₐ₂, R_{b1}, and R_{b2} is CH₃, and Rₐ₃ and R_{b3} are

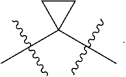 [SMILES: *C1(*)CC1]

In some variations of formula (12) described in the paragraphs above in which o₂ is 3, each of Rₐ₁ and Rₐ₂ is H and each of R_{b1} and R_{b2} is CH₃, and Rₐ₃ and R_{b3} are

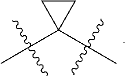 [SMILES: *C1(*)CC1]

In some variations of formula (12) described in the paragraphs above in which o₂ is 3, each of Rₐ₁, R_{b1}, Rₐ₂, and R_{b2}, is H and Rₐ₃ and R_{b3} are

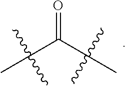 [SMILES: *C(*)=O]

In some variations of formula (12) described in the paragraphs above in which o₂ is 3, each of Rₐ₁, R_{b1}, Rₐ₂, and R_{b2}, is CH₃ and Rₐ₃ and R_{b3} are

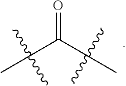 [SMILES: *C(*)=O]

In some variations of formula (12) described in the paragraphs above in which o₂ is 3, Rₐ₁ is H and each of Rₐ₂, R_{b1}, and R_{b2} is CH₃, and Rₐ₃ and R_{b3} are

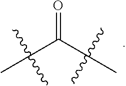 [SMILES: *C(*)=O]

In some variations of formula (12) described in the paragraphs above in which o₂ is 3, each of Rₐ₁ and Rₐ₂ is H and each of R_{b1} and R_{b2} is CH₃, and Rₐ₃ and R_{b3} are

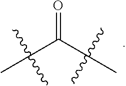 [SMILES: *C(*)=O]

In some variations of formula (12) described in the paragraphs above in which o₂ is 3, Rₐ₁ and R_{b1} are

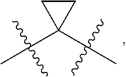 [SMILES: *C1(*)CC1]

Rₐ₂ and R_{b2} are

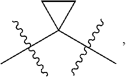 [SMILES: *C1(*)CC1]

and each of Rₐ₃ and R_{b3} is H. In some variations of formula (12) described in the paragraphs above in which o₂ is 3, Rₐ₁ and R_{b1} are

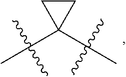 [SMILES: *C1(*)CC1]

Rₐ₂ and R_{b2} are

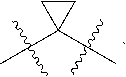 [SMILES: *C1(*)CC1]

and each of Rₐ₃ and R_{b3} is CH₃. In some variations of formula (12) described in the paragraphs above in which o₂ is 3, Rₐ₁ and R_{b1} are

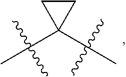 [SMILES: *C1(*)CC1]

Rₐ₂ and R_{b2} are

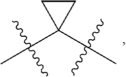 [SMILES: *C1(*)CC1]

Rₐ₃ is H, and R_{b3} is CH₃. In some variations of formula (12) described in the paragraphs above in which o₂ is 3, Rₐ₁ and R_{b1} are

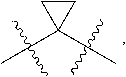 [SMILES: *C1(*)CC1]

Rₐ₂ and R_{b2} are

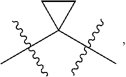 [SMILES: *C1(*)CC1]

and each of Rₐ₃ and R_{b3} is H. In some variations of formula (12) described in the paragraphs above in which o₂ is 3, Rₐ₁ and R_{b1} are

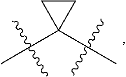 [SMILES: *C1(*)CC1]

Rₐ₂ and R_{b2} are

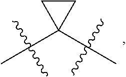 [SMILES: *C1(*)CC1]

and each of Rₐ₃ and R_{b3} is CH₃. In some variations of formula (12) described in the paragraphs above in which o₂ is 3, Rₐ₁ and R_{b1} are

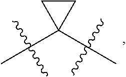 [SMILES: *C1(*)CC1]

Rₐ₂ and R_{b2} are

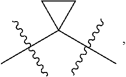 [SMILES: *C1(*)CC1]

Rₐ₃ is H, and R_{b3} is CH₃.

In some variations of formula (12) described in the paragraphs above in which o₂ is 3, Rₐ₁ and R_{b1} are

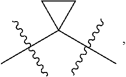 [SMILES: *C1(*)CC1]

Rₐ₂ and R_{b2} are

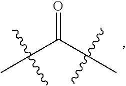 [SMILES: *C(*)=O]

and each of Rₐ₃ and R_{b3} is H. In some variations of formula (12) described in the paragraphs above in which o₂ is 3, Rₐ₁ and R_{b1} are

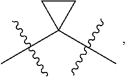 [SMILES: *C1(*)CC1]

Rₐ₂ and R_{b2} are

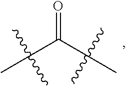 [SMILES: *C(*)=O]

and each of Rₐ₃ and R_{b3} is CH₃. In some variations of formula (12) described in the paragraphs above in which o₂ is 3, Rₐ₁ and R_{b1} are

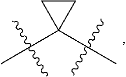 [SMILES: *C1(*)CC1]

Rₐ₂ and R_{b2} are

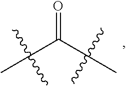 [SMILES: *C(*)=O]

Rₐ₃ is H, and R_{b3} is CH₃. In some variations of formula (12) described in the paragraphs above in which o₂ is 3, Rₐ₁ and R_{b1} are

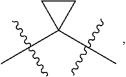 [SMILES: *C1(*)CC1]

Rₐ₂ and R_{b2} are

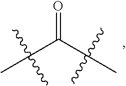 [SMILES: *C(*)=O]

and each of Rₐ₃ and R_{b3} is H. In some variations of formula (12) described in the paragraphs above in which o₂ is 3, Rₐ₁ and R_{b1} are

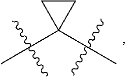 [SMILES: *C1(*)CC1]

Rₐ₂ and R_{b2} are

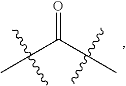 [SMILES: *C(*)=O]

and each of Rₐ₃ and R_{b3} is CH₃. In some variations of formula (12) described in the paragraphs above in which o₂ is 3, Rₐ₁ and R_{b1} are

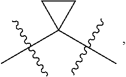 [SMILES: *C1(*)CC1]

Rₐ₂ and R_{b2} are

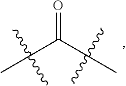 [SMILES: *C(*)=O]

Rₐ3 is H, and R_{b3} is CH₃.

In some variations of formula (12) described in the paragraphs above in which o₂ is 3, Rₐ₁ and R_{b1} are

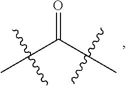 [SMILES: *C(*)=O]

Rₐ₂ and R_{b2} are

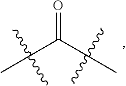 [SMILES: *C(*)=O]

and each of Rₐ₃ and R_{b3} is H. In some variations of formula (12) described in the paragraphs above in which o₂ is 3, Rₐ₁ and R_{b1} are

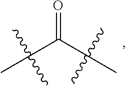 [SMILES: *C(*)=O]

Rₐ₂ and R_{b2} are

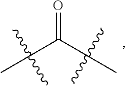 [SMILES: *C(*)=O]

and each of Rₐ₃ and R_{b3} is CH₃. In some variations of formula (12) described in the paragraphs above in which o₂ is 3, Rₐ₁ and R_{b1} are

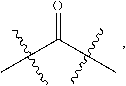 [SMILES: *C(*)=O]

Rₐ₂ and R_{b2} are

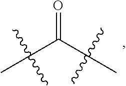 [SMILES: *C(*)=O]

Rₐ₃ is H, and R_{b3} is CH₃. In some variations of formula (12) described in the paragraphs above in which o₂ is 3, Rₐ₁ and R_{b1} are

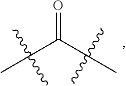 [SMILES: *C(*)=O]

Rₐ₂ and R_{b2} are

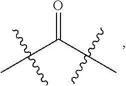 [SMILES: *C(*)=O]

and each of Rₐ₃ and R_{b3} is H. In some variations of formula (12) described in the paragraphs above in which o₂ is 3, Rₐ₁ and R_{b1} are

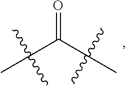 [SMILES: *C(*)=O]

Rₐ₂ and R_{b2} are

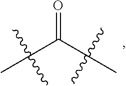 [SMILES: *C(*)=O]

and each of Rₐ₃ and R_{b3} is CH₃. In some variations of formula (12) described in the paragraphs above in which o₂ is 3, Rₐ₁ and R_{b1} are

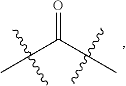 [SMILES: *C(*)=O]

Rₐ₂ and R_{b2} are

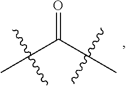 [SMILES: *C(*)=O]

Rₐ₃ is H, and R_{b3} is CH₃.

In some variations of formula (12) described in the paragraphs above in which o₂ is 3, Rₐ₁ and R_{b1} and Rₐ₂ and R_{b2} together are

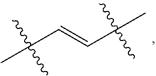 [SMILES: */C=C/*]

and each of Rₐ₃ and R_{b3} is H. In some variations of formula (12) described in the paragraphs above in which o₂ is 3, Rₐ₁ and R_{b1} and Rₐ₂ and R_{b2} together are

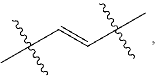 [SMILES: */C=C/*]

and each of Rₐ₃ and R_{b3} is CH₃. In some variations of formula (12) described in the paragraphs above in which o₂ is 3, Rₐ₁ and R_{b1} and Rₐ₂ and R_{b2} together are

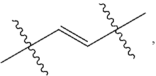 [SMILES: */C=C/*]

Rₐ₃ is H, and R_{b3} is CH₃. In some variations of formula (12) described in the paragraphs above in which o₂ is 3, Rₐ₁ and R_{b1} and Rₐ₂ and R_{b2} together are

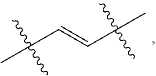 [SMILES: */C=C/*]

and Rₐ₃ and R_{b3} are

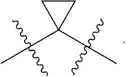 [SMILES: *C1(*)CC1]

In some variations of formula (12) described in the paragraphs above in which o₂ is 3, Rₐ₁ and R_{b1} and Rₐ₂ and R_{b2} together are

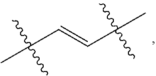 [SMILES: */C=C/*]

and Rₐ₃ and R_{b3} are

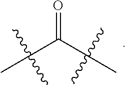 [SMILES: *C(*)=O]

In variations of formula (12) described in the paragraphs above in which o₂ is 4, the three instances of Rₐ and R_{b} are indicated as Rₐ₁ and R_{b1}; Rₐ₂ and R_{b2}; Rₐ₃ and R_{b3}; and Rₐ₄ and R_{b4}, respectively. In some variations of formula (12) described in the paragraphs above in which o₂ is 4, In some variations of formula (12) described in the paragraphs above in which o₂ is 4, each of Rₐ₁, R_{b1}, Rₐ₂, R_{b2}, Rₐ₃, R_{b3}, Rₐ₄, and R_{b4} is H. In some variations of formula (12) described in the paragraphs above in which o₂ is 4, each of Rₐ₁, R_{b1}, Rₐ₂, R_{b2}, Rₐ₃, R_{b3}, Rₐ₄, and R_{b4} is CH₃. In some variations of formula (12) described in the paragraphs above in which o₂ is 4, each of Rₐ₁, R_{b1}, Rₐ₂, R_{b2}, Rₐ₃, and R_{b3} is H and each of Rₐ₄ and R_{b4} is CH₃. In some variations of formula (12) described in the paragraphs above in which o₂ is 4, each of Rₐ₁, R_{b1}, Rₐ₂, and R_{b2} is H and each of Rₐ₃, R_{b3}, Rₐ₄, and R_{b4} is CH₃. In some variations of formula (12) described in the paragraphs above in which o₂ is 4, each of Rₐ₁, R_{b1}, Rₐ₂, R_{b2}, Rₐ₃, and R_{b3} is CH₃ and each of Rₐ₄ and R_{b4} is H.

In some variations of formula (12) described in the paragraphs above in which o₂ is 4, each of Rₐ₁, R_{b1}, and Rₐ₃ is H and each of R_{b1}, R_{b2}, and R_{b3} is CH₃. In some variations of formula (12) described in the paragraphs above in which o₂ is 4, Rₐ₁ is H, R_{b1} is CH₃, and each of Rₐ₂, R_{b2}, Rₐ₃, and R_{b3} is CH₃. In some variations of formula (12) described in the paragraphs above in which o₂ is 4, Rₐ₁ is H, R_{b1} is CH₃, and each of Rₐ₂, R_{b2}, Rₐ₃, and R_{b3} is H. In some variations of formula (12) described in the paragraphs above in which O₂ is 4, each of Rₐ₁ and Rₐ₂ is H, each of R_{b1} and R_{b2} is CH₃, and each of Rₐ₃ and R_{b3} is H. In some variations of formula (12) described in the paragraphs above in which o₂ is 4, each of Rₐ₁ and Rₐ₂ is H, each of R_{b1} and R_{b2} is CH₃, and each of Rₐ₃ and R_{b3} is CH₃.

In some variations of formula (12) described in the paragraphs above in which o₂ is 4, Rₐ₁ and R_{b1} are H, Rₐ₂ and R_{b2} are H, Rₐ₃ and R_{b3} are H, and Rₐ₄ and R_{b4} are

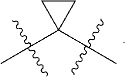 [SMILES: *C1(*)CC1]

In some variations of formula (12) described in the paragraphs above in which o₂ is 4, Rₐ₁ and R_{b1} are H, Rₐ₂ and R_{b2} are H, Rₐ₃ and R_{b3} are CH₃, and Rₐ₄ and R_{b4} are

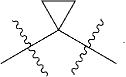 [SMILES: *C1(*)CC1]

In some variations of formula (12) described in the paragraphs above in which o₂ is 4, Rₐ₁ and R_{b1} are H, Rₐ₂ and R_{b2} are CH₃, Rₐ₃ and R_{b3} are CH₃, and Rₐ₄ and R_{b4} are

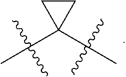 [SMILES: *C1(*)CC1]

In some variations of formula (12) described in the paragraphs above in which o₂ is 4, Rₐ₁ and R_{b1} are CH₃, Rₐ₂ and R_{b2} are CH₃, Rₐ₃ and R_{b3} are CH₃, and Rₐ₄ and R_{b4} are

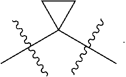 [SMILES: *C1(*)CC1]

In some variations of formula (12) described in the paragraphs above in which o₂ is 4, Rₐ₁ and R_{b1} are H, Rₐ₂ and R_{b2} are H, Rₐ₃ and R_{b3} are

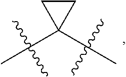 [SMILES: *C1(*)CC1]

and Rₐ₄ and R_{b4} are

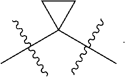 [SMILES: *C1(*)CC1]

In some variations of formula (12) described in the paragraphs above in which o₂ is 4, Rₐ₁ and R_{b1} are H, Rₐ₂ and R_{b2} are CH₃, Rₐ₃ and R_{b3} are

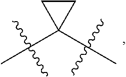 [SMILES: *C1(*)CC1]

and Rₐ₄ and R_{b4} are

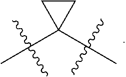 [SMILES: *C1(*)CC1]

In some variations of formula (12) described in the paragraphs above in which o₂ is 4, Rₐ₁ and R_{b1} are CH₃, Rₐ₂ and R_{b2} are CH₃, Rₐ₃ and R_{b3} are

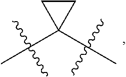 [SMILES: *C1(*)CC1]

and Rₐ₄ and R_{b4} are

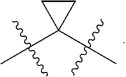 [SMILES: *C1(*)CC1]

In some variations of formula (12) described in the paragraphs above in which o₂ is 4, Rₐ₁ is H, R_{b1} is CH₃, Rₐ₂ and R_{b2} are CH₃, Rₐ₃ and R_{b3} are

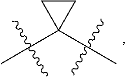 [SMILES: *C1(*)CC1]

and Rₐ₄ and R_{b4} are

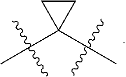 [SMILES: *C1(*)CC1]

In some variations of formula (12) described in the paragraphs above in which o₂ is 4, Rₐ₁ is H, R_{b1} is CH₃, Rₐ₂ is H, R_{b2} is CH₃, Rₐ₃ and R_{b3} are

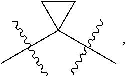 [SMILES: *C1(*)CC1]

and Rₐ₄ and R_{b4} are

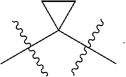 [SMILES: *C1(*)CC1]

In some variations of formula (12) described in the paragraphs above in which o₂ is 4, Rₐ₁ and R_{b1} are H, Rₐ₂ and R_{b2} are

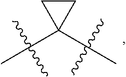 [SMILES: *C1(*)CC1]

Rₐ₃ and R_{b3} are

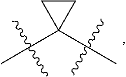 [SMILES: *C1(*)CC1]

and Rₐ₄ and R_{b4} are

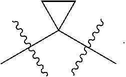 [SMILES: *C1(*)CC1]

In some variations of formula (12) described in the paragraphs above in which o₂ is 4, Rₐ₁ and R_{b1} are CH₃, Rₐ₂ and R_{b2} are

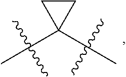 [SMILES: *C1(*)CC1]

Rₐ₃ and R_{b3} are

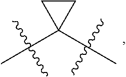 [SMILES: *C1(*)CC1]

and Rₐ₄ and R_{b4} are

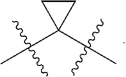 [SMILES: *C1(*)CC1]

In some variations of formula (12) described in the paragraphs above in which o₂ is 4, Rₐ₁ is H, R_{b1} is CH₃, Rₐ₂ and R_{b2} are

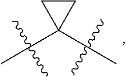 [SMILES: *C1(*)CC1]

Rₐ₃ and R_{b3} are

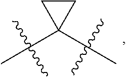 [SMILES: *C1(*)CC1]

and Rₐ₄ and R_{b4} are

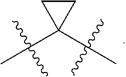 [SMILES: *C1(*)CC1]

In some variations of formula (12) described in the paragraphs above in which o₂ is 4, Rₐ₁ and R_{b1} are

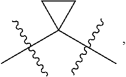 [SMILES: *C1(*)CC1]

Rₐ₂ and R_{b2} are

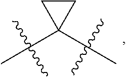 [SMILES: *C1(*)CC1]

Rₐ₃ and R_{b3} are

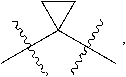 [SMILES: *C1(*)CC1]

and Rₐ₄ and R_{b4} are

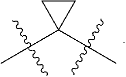 [SMILES: *C1(*)CC1]

In some variations of formula (12) described in the paragraphs above in which o₂ is 4, Rₐ₁ and R_{b1} are H, Rₐ₂ and R_{b2} are H, Rₐ₃ and R_{b3} are H, and Rₐ₄ and R_{b4} are

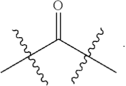 [SMILES: *C(*)=O]

In some variations of formula (12) described in the paragraphs above in which o₂ is 4, Rₐ₁ and R_{b1} are H, Rₐ₂ and R_{b2} are H, Rₐ₃ and R_{b3} are CH₃, and Rₐ₄ and R_{b4} are

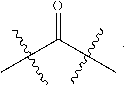 [SMILES: *C(*)=O]

In some variations of formula (12) described in the paragraphs above in which o₂ is 4, Rₐ₁ and R_{b1} are H, Rₐ₂ and R_{b2} are CH₃, Rₐ₃ and R_{b3} are CH₃, and Rₐ₄ and R_{b4} are

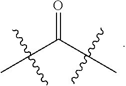 [SMILES: *C(*)=O]

In some variations of formula (12) described in the paragraphs above in which o₂ is 4, Rₐ₁ and R_{b1} are CH₃, Rₐ₂ and R_{b2} are CH₃, Rₐ₃ and R_{b3} are CH₃, and Rₐ₄ and R_{b4} are

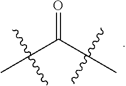 [SMILES: *C(*)=O]

In some variations of formula (12) described in the paragraphs above in which o₂ is 4, Rₐ₁ and R_{b1} are H, Rₐ₂ and R_{b2} are H, Rₐ₃ and R_{b3} are

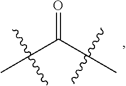 [SMILES: *C(*)=O]

and Rₐ₄ and R_{b4} are v. In some variations of formula (12) described in the paragraphs above in which o₂ is 4, Rₐ₁ and R_{b1} are H, Rₐ₂ and R_{b2} are CH₃, Rₐ₃ and R_{b3} are

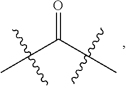 [SMILES: *C(*)=O]

and Rₐ₄ and R_{b4} are

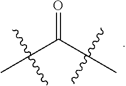 [SMILES: *C(*)=O]

In some variations of formula (12) described in the paragraphs above in which o₂ is 4, Rₐ₁ and R_{b1} are CH₃, Rₐ₂ and R_{b2} are CH₃, Rₐ₃ and R_{b3} are

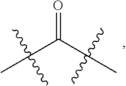 [SMILES: *C(*)=O]

and Rₐ₄ and R_{b4} are

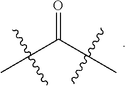 [SMILES: *C(*)=O]

In some variations of formula (12) described in the paragraphs above in which o₂ is 4, Rₐ₁ is H, R_{b1} is CH₃, Rₐ₂ and R_{b2} are CH₃, Rₐ₃ and R_{b3} are

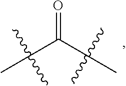 [SMILES: *C(*)=O]

and Rₐ₄ and R_{b4} are

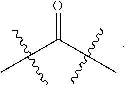 [SMILES: *C(*)=O]

In some variations of formula (12) described in the paragraphs above in which o₂ is 4, Rₐ₁ is H, R_{b1} is CH₃, Rₐ₂ is H, R_{b2} is CH₃, Rₐ₃ and R_{b3} are

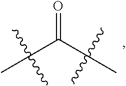 [SMILES: *C(*)=O]

and Rₐ₄ and R_{b4} are

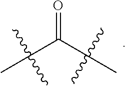 [SMILES: *C(*)=O]

In some variations of formula (12) described in the paragraphs above in which o₂ is 4, Rₐ₁ and R_{b1} are H, Rₐ₂ and R_{b2} are

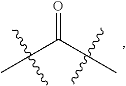 [SMILES: *C(*)=O]

Rₐ₃ and R_{b3} are

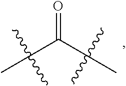 [SMILES: *C(*)=O]

and Rₐ₄ and R_{b4} are

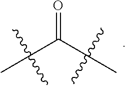 [SMILES: *C(*)=O]

In some variations of formula (12) described in the paragraphs above in which o₂ is 4, Rₐ₁ and R_{b1} are CH₃, Rₐ₂ and R_{b2} are

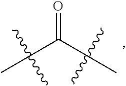 [SMILES: *C(*)=O]

Rₐ₃ and R_{b3} are

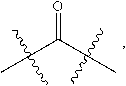 [SMILES: *C(*)=O]

and Rₐ₄ and R_{b4} are

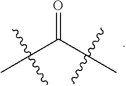 [SMILES: *C(*)=O]

In some variations of formula (12) described in the paragraphs above in which o₂ is 4, Rₐ₁ is H, R_{b1} is CH₃, Rₐ₂ and R_{b2} are

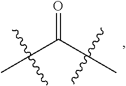 [SMILES: *C(*)=O]

Rₐ₃ and R_{b3} are

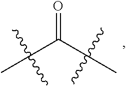 [SMILES: *C(*)=O]

and Rₐ₄ and R_{b4} are

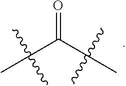 [SMILES: *C(*)=O]

In some variations of formula (12) described in the paragraphs above in which o₂ is 4, Rₐ₁ and R_{b1} are

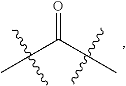 [SMILES: *C(*)=O]

Rₐ₂ and R_{b2} are

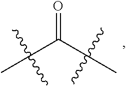 [SMILES: *C(*)=O]

Rₐ₃ and R_{b3} are

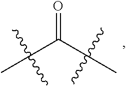 [SMILES: *C(*)=O]

and Rₐ₄ and R_{b4} are

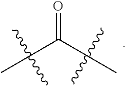 [SMILES: *C(*)=O]

In some variations of formula (12) described in the paragraphs above in which o₂ is 4, Rₐ₁ and R_{b1} are

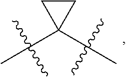 [SMILES: *C1(*)CC1]

Rₐ₂ and R_{b2} are H, Rₐ₃ and R_{b3} are H, and Rₐ₄ and R_{b4} are

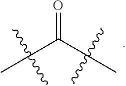 [SMILES: *C(*)=O]

In some variations of formula (12) described in the paragraphs above in which o₂ is 4, Rₐ₁ and R_{b1} are

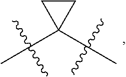 [SMILES: *C1(*)CC1]

Rₐ₂ and R_{b2} are CH₃, Rₐ₃ and R_{b3} are CH₃, and Rₐ₄ and R_{b4} are

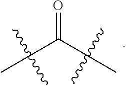 [SMILES: *C(*)=O]

In some variations of formula (12) described in the paragraphs above in which o₂ is 4, Rₐ₁ and R_{b1} are

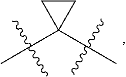 [SMILES: *C1(*)CC1]

Rₐ₂ and R_{b2} are H, Rₐ₃ and R_{b3} are CH₃, and Rₐ₄ and R_{b4} are

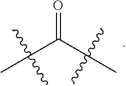 [SMILES: *C(*)=O]

In some variations of formula (12) described in the paragraphs above in which o₂ is 4, Rₐ₁ and R_{b1} are

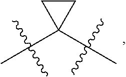 [SMILES: *C1(*)CC1]

Rₐ₂ and R_{b2} are

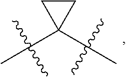 [SMILES: *C1(*)CC1]

Rₐ₃ and R_{b3} are H, and Rₐ₄ and R_{b4} are

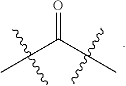 [SMILES: *C(*)=O]

In some variations of formula (12) described in the paragraphs above in which o₂ is 4, Rₐ₁ and R_{b1} are

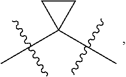 [SMILES: *C1(*)CC1]

Rₐ₂ and R_{b2} are

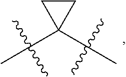 [SMILES: *C1(*)CC1]

Rₐ₃ and R_{b3} are CH₃, and Rₐ₄ and R_{b4} are

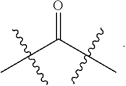 [SMILES: *C(*)=O]

In some variations of formula (12) described in the paragraphs above in which o₂ is 4, Rₐ₁ and R_{b1} are

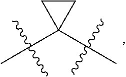 [SMILES: *C1(*)CC1]

Rₐ₂ and R_{b2} are

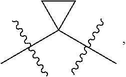 [SMILES: *C1(*)CC1]

Rₐ₃ and R_{b3} are CH₃, and Rₐ₄ and R_{b4} are

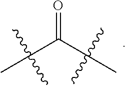 [SMILES: *C(*)=O]

In some variations of formula (12) described in the paragraphs above in which o₂ is 4, Rₐ₁ and R_{b1} are

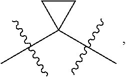 [SMILES: *C1(*)CC1]

Rₐ₂ and R_{b2} are

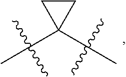 [SMILES: *C1(*)CC1]

Rₐ₃ and R_{b3} are H, and Rₐ₄ and R_{b4} are

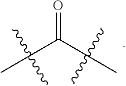 [SMILES: *C(*)=O]

In some variations of formula (12) described in the paragraphs above in which o₂ is 4, Rₐ₁ and R_{b1} are

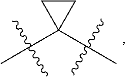 [SMILES: *C1(*)CC1]

Rₐ₂ and R_{b2} are

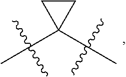 [SMILES: *C1(*)CC1]

Rₐ₃ and R_{b3} are

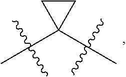 [SMILES: *C1(*)CC1]

and Rₐ₄ and R_{b4} are

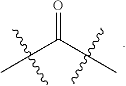 [SMILES: *C(*)=O]

In some variations of formula (12) described in the paragraphs above in which o₂ is 4, Rₐ₁ and R_{b1} are

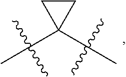 [SMILES: *C1(*)CC1]

Rₐ₂ and R_{b2} are

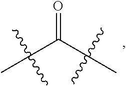 [SMILES: *C(*)=O]

Rₐ₃ and R_{b3} are H, and Rₐ₄ and R_{b4} are

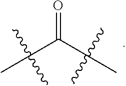 [SMILES: *C(*)=O]

In some variations of formula (12) described in the paragraphs above in which o₂ is 4, Rₐ₁ and R_{b1} are

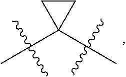 [SMILES: *C1(*)CC1]

Rₐ₂ and R_{b2} are

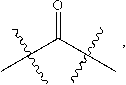 [SMILES: *C(*)=O]

Rₐ₃ and R_{b3} are CH₃, and Rₐ₄ and R_{b4} are

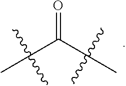 [SMILES: *C(*)=O]

In some variations of formula (12) described in the paragraphs above in which o₂ is 4, Rₐ₁ and R_{b1} are

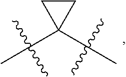 [SMILES: *C1(*)CC1]

Rₐ₂ and R_{b2} are

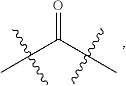 [SMILES: *C(*)=O]

Rₐ₃ and R_{b3} are CH₃, and Rₐ₄ and R_{b4} are

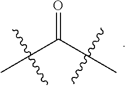 [SMILES: *C(*)=O]

In some variations of formula (12) described in the paragraphs above in which o₂ is 4, Rₐ₁ and R_{b1} are

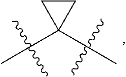 [SMILES: *C1(*)CC1]

Rₐ₂ and R_{b2} are

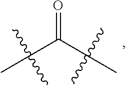 [SMILES: *C(*)=O]

Rₐ₃ and R_{b3} are H, and Rₐ₄ and R_{b4} are

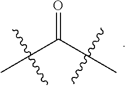 [SMILES: *C(*)=O]

In some variations of formula (12) described in the paragraphs above in which o₂ is 4, Rₐ₁ and R_{b1} are

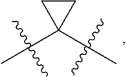 [SMILES: *C1(*)CC1]

Rₐ₂ and R_{b2} are

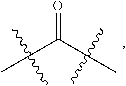 [SMILES: *C(*)=O]

Rₐ₃ and R_{b3} are

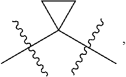 [SMILES: *C1(*)CC1]

and Rₐ₄ and R_{b4} are

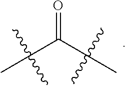 [SMILES: *C(*)=O]

In some variations of formula (12) described in the paragraphs above in which o₂ is 4, Rₐ₁ and R_{b1} are

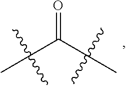 [SMILES: *C(*)=O]

Rₐ₂ and R_{b2} are

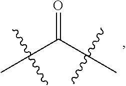 [SMILES: *C(*)=O]

Rₐ₃ and R_{b3} are H, and Rₐ₄ and R_{b4} are

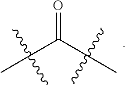 [SMILES: *C(*)=O]

In some variations of formula (12) described in the paragraphs above in which o₂ is 4, Rₐ₁ and R_{b1} are

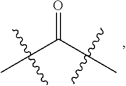 [SMILES: *C(*)=O]

Rₐ₂ and R_{b2} are

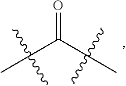 [SMILES: *C(*)=O]

Rₐ₃ and R_{b3} are CH₃, and Rₐ₄ and R_{b4} are

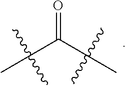 [SMILES: *C(*)=O]

In some variations of formula (12) described in the paragraphs above in which o₂ is 4, Rₐ₁ and R_{b1} are

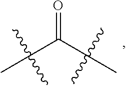 [SMILES: *C(*)=O]

Rₐ₂ and R_{b2} are

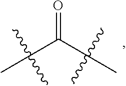 [SMILES: *C(*)=O]

Rₐ₃ and R_{b3} are CH₃, and Rₐ₄ and R_{b4} are

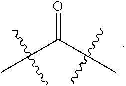 [SMILES: *C(*)=O]

In some variations of formula (12) described in the paragraphs above in which o₂ is 4, Rₐ₁ and R_{b1} are

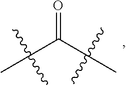 [SMILES: *C(*)=O]

Rₐ₂ and R_{b2} are

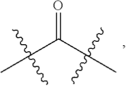 [SMILES: *C(*)=O]

Rₐ₃ and R_{b3} are H, and Rₐ₄ and R_{b4} are

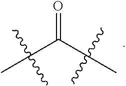 [SMILES: *C(*)=O]

In some variations of formula (12) described in the paragraphs above in which o₂ is 4, Rₐ₁ and R_{b1} are

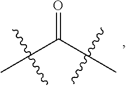 [SMILES: *C(*)=O]

Rₐ₂ and R_{b2} are

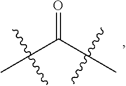 [SMILES: *C(*)=O]

Rₐ₃ and R_{b3} are

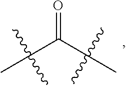 [SMILES: *C(*)=O]

and Rₐ₄ and R_{b4} are

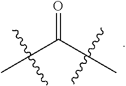 [SMILES: *C(*)=O]

In some variations of formula (12) described in the paragraphs above in which o₂ is 4, Rₐ₁ and R_{b1} are H, Rₐ₂ and R_{b2} are H, and Rₐ₃ and R_{b3} and Rₐ₄ and R_{b4} together are

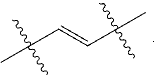 [SMILES: */C=C/*]

In some variations of formula (12) described in the paragraphs above in which O₂ is 4, Rₐ₁ and R_{b1} are H, Rₐ₂ and R_{b2} are CH₃, and Rₐ₃ and R_{b3} and Rₐ₄ and R_{b4} together are

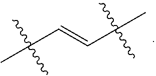 [SMILES: */C=C/*]

In some variations of formula (12) described in the paragraphs above in which o₂ is 4, Rₐ₁ and Rₐ₂ are H, R_{b1} and R_{b2} are CH₃, and Rₐ₃ and R_{b3} and Rₐ₄ and R_{b4} together are

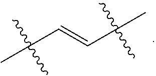 [SMILES: */C=C/*]

In some variations of formula (12) described in the paragraphs above in which o₂ is 4, Rₐ₁ and R_{b1} are H, Rₐ₂ and R_{b2} are

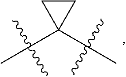 [SMILES: *C1(*)CC1]

and Rₐ₃ and R_{b3} and Rₐ₄ and R_{b4} together are

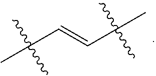 [SMILES: */C=C/*]

In some variations of formula (12) described in the paragraphs above in which o₂ is 4, Rₐ₁ and R_{b1} are

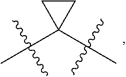 [SMILES: *C1(*)CC1]

Rₐ₂ and R_{b2} are CH₃, and Rₐ₃ and R_{b3} and Rₐ₄ and R_{b4} together are

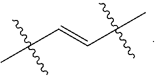 [SMILES: */C=C/*]

In some variations of formula (12) described in the paragraphs above in which o₂ is 4, Rₐ₁ is H, R_{b1} is CH₃, Rₐ₂ and R_{b2} are

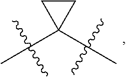 [SMILES: *C1(*)CC1]

and Rₐ₃ and R_{b3} and Rₐ₄ and R_{b4} together are

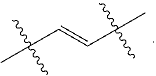 [SMILES: */C=C/*]

In some variations of formula (12) described in the paragraphs above in which o₂ is 4, Rₐ₁ and R_{b1} are H, Rₐ₂ and R_{b2} are

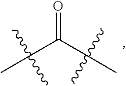 [SMILES: *C(*)=O]

and Rₐ₃ and R_{b3} and Rₐ₄ and R_{b4} together are

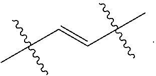 [SMILES: */C=C/*]

In some variations of formula (12) described in the paragraphs above in which o₂ is 4, Rₐ₁ and R_{b1} are

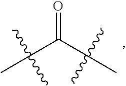 [SMILES: *C(*)=O]

Rₐ₂ and R_{b2} are CH₃, and Rₐ₃ and R_{b3} and Rₐ₄ and R_{b4} together are

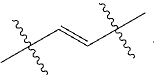 [SMILES: */C=C/*]

In some variations of formula (12) described in the paragraphs above in which o₂ is 4, Rₐ₁ is H, R_{b1} is CH₃, Rₐ₂ and R_{b2} are

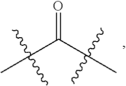 [SMILES: *C(*)=O]

and Rₐ₃ and R_{b3} and Rₐ₄ and R_{b4} together are

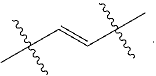 [SMILES: */C=C/*]

In some variations of formula (12) described in the paragraphs above in which o₂ is 4, Rₐ₁ and R_{b1} are

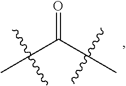 [SMILES: *C(*)=O]

Rₐ₂ and R_{b2} are

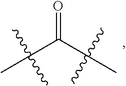 [SMILES: *C(*)=O]

and Rₐ₃ and R_{b3} and Rₐ₄ and R_{b4} together are

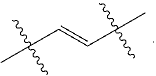 [SMILES: */C=C/*]

In some variations of formula (12) described in the paragraphs above in which o₂ is 4, Rₐ₁ and R_{b1} are

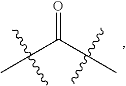 [SMILES: *C(*)=O]

Rₐ₂ and R_{b2} are

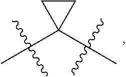 [SMILES: *C1(*)CC1]

and Rₐ₃ and R_{b3} and Rₐ₄ and R_{b4} together are

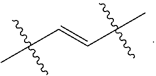 [SMILES: */C=C/*]

In some variations of formula (12) described in the paragraphs above in which o₂ is 4, Rₐ₁ and R_{b1} are

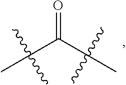 [SMILES: *C(*)=O]

Rₐ₂ and R_{b2} are

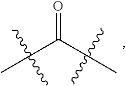 [SMILES: *C(*)=O]

and Rₐ₃ and R_{b3} and Rₐ₄ and R_{b4} together are

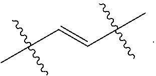 [SMILES: */C=C/*]

In some variations of formula (12) described in the paragraphs above in which o₂ is 4, Rₐ₁ and R_{b1} are

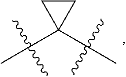 [SMILES: *C1(*)CC1]

Rₐ₂ and R_{b2} are

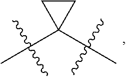 [SMILES: *C1(*)CC1]

and Rₐ₃ and R_{b3} and Rₐ₄ and R_{b4} together are

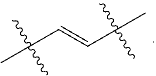 [SMILES: */C=C/*]

In some variations of formula (12) described in the paragraphs above in which o₂ is 4, Rₐ₁ and R_{b1} and Rₐ₂ and R_{b2} together are

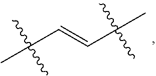 [SMILES: */C=C/*]

and Rₐ₃ and R_{b3} and Rₐ₄ and R_{b4} together are

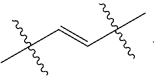 [SMILES: */C=C/*]

In some variations of formula (12) described in the paragraphs above, ring A is:

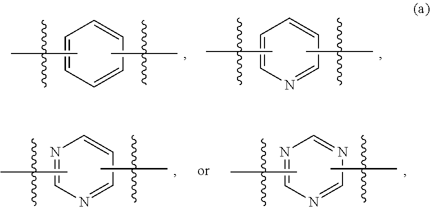

optionally substituted with halo or C1-C6 linear or branched alkyl;

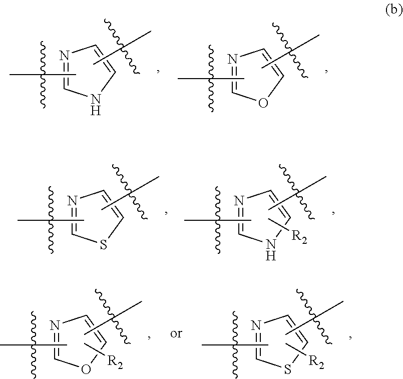

optionally substituted with halo or C1-C6 linear or branched alkyl; or

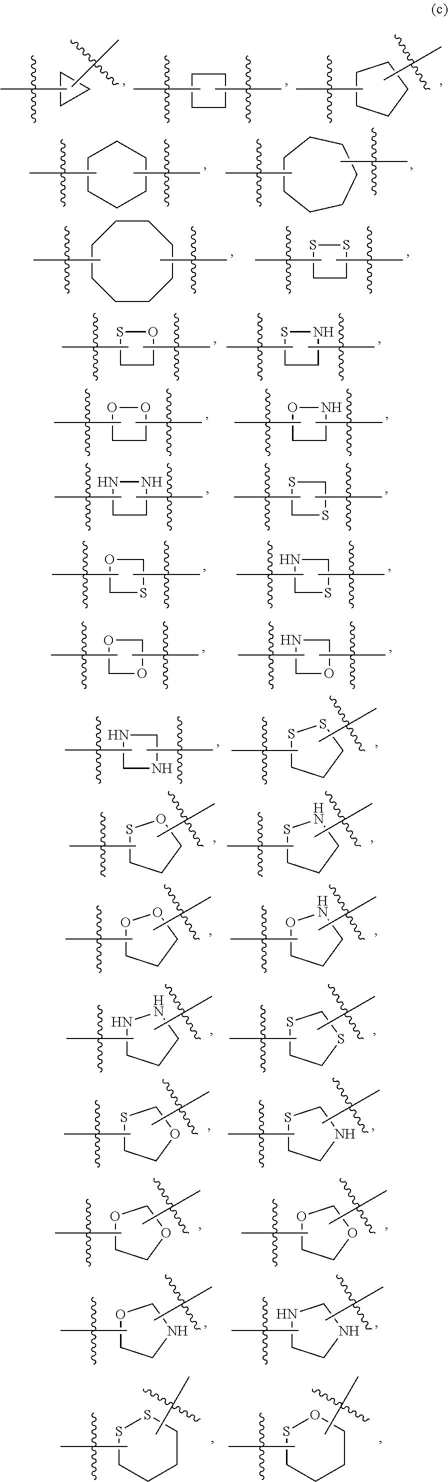

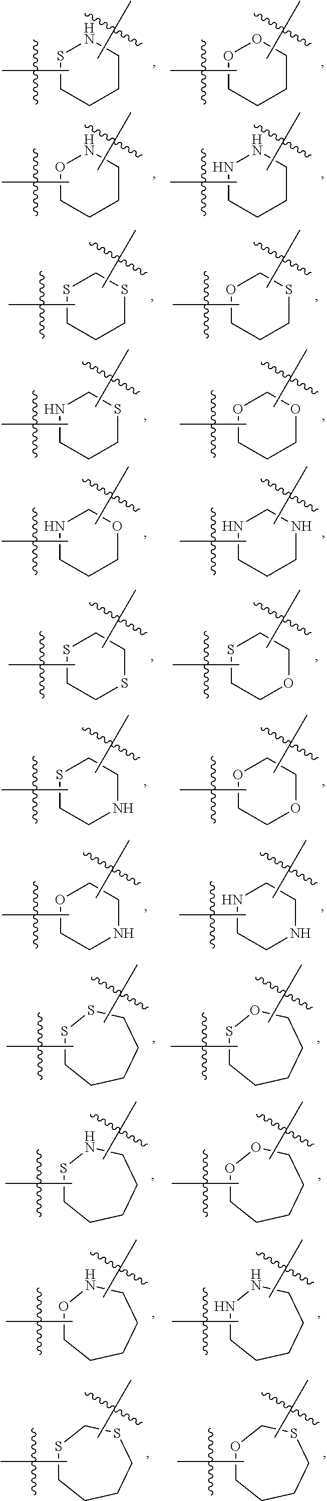

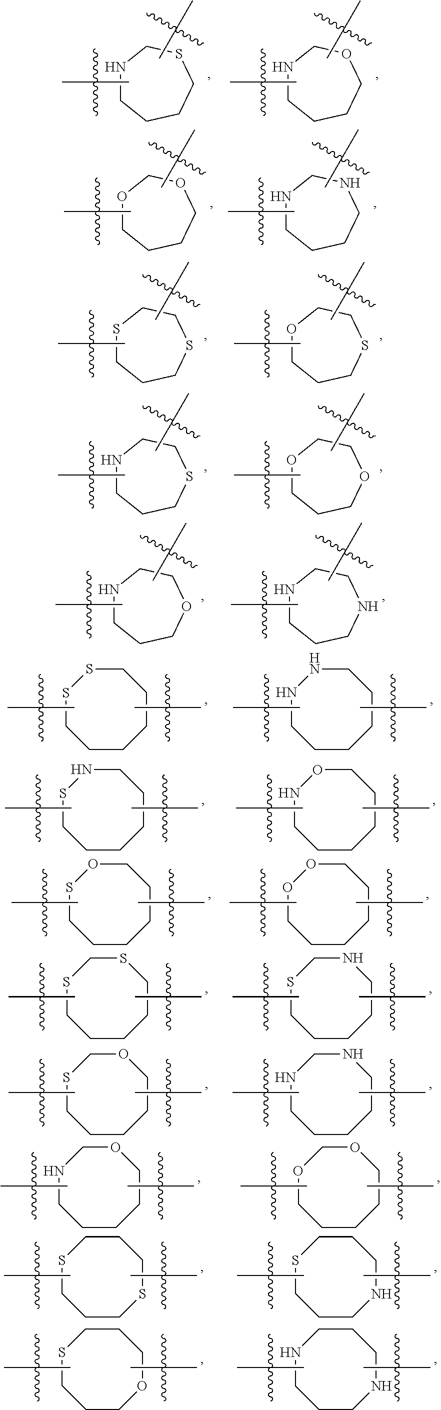

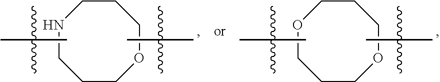

optionally substituted with halo or C1-C6 linear or branched alkyl.

In some variations of formula (12) described in the paragraphs above, ring A is substituted with halo. In some variations, the halo is F, Br, I, or Cl.

In some variations of formula (12) described in the paragraphs above, ring A is substituted with C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (12) described in the paragraphs above, n is 1-4, 1-3, 1-2, 2-4, 2-3, 3-4, 1, 2, 3, or 4.

In some variations of formula (12) described in the paragraphs above, D1 is safranin-O. In some variations of formula (12) described in the paragraphs above, D2 is safranin-O. In some variations of formula (12) described in the paragraphs above, D1 and D2 are safranin-O.

In some variations of formula (12) described in the paragraph above, the pendant phenyl ring of D1 is unsubstituted. In some variations of formula (12) described in the paragraph above, the pendant phenyl ring of D1 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D1 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D1 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (12) described in the paragraph above, the pendant phenyl ring of D2 is unsubstituted. In some variations of formula (12) described in the paragraph above, the pendant phenyl ring of D2 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D2 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D2 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some of the variations of formula (12) described above, D1 and D2 are safranin-O moieties, as shown in formula (12a):

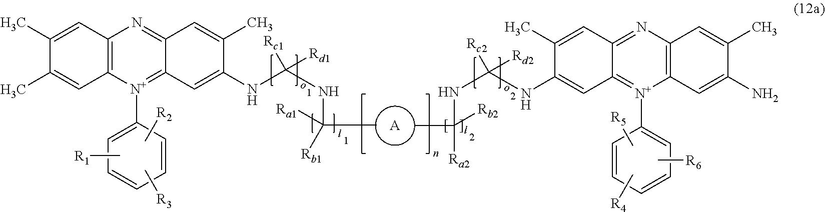

in which l₁, l₂, n, o₁, o₂, ring A, Rₐ₁, R_{b1}, Rₐ₂, R_{b2}, R_{c2}, R_{d1}, Rₐ, and R_{d2} are as described in the paragraphs above, R₁, R₂, R₃, R₄, R₅, and R₆ independently are absent or independently are selected from —NH₂, —NHR, —NR₂, —OH, —O⁻, —NHCOCH₃, —NHCOR, —OCH₃, —OR, —C₂H₅, —R, —C₆H₅, —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR), and R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

Some cationic dye dimers fall within formula (12b):

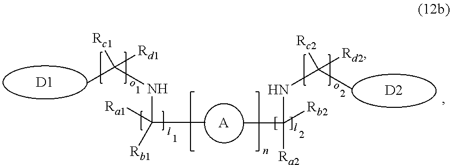

in which each of D1 and D2 is a cationic dye moiety; l₁, l₂, n, independently are 1-4; o₁ and o₂ independently are 1-8; ring A is aryl, heteroaryl, cycloalkyl, or heterocyclyl; for each independent instance of Rₐ₁ and R_{b1}, Rₐ₁ and R_{b1} (1) independently are H or CH₃, or (2) Rₐ₁ and R_{b1} are

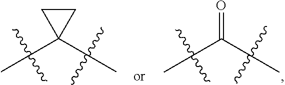

or (3) two of CRₐ₁R_{b1} are

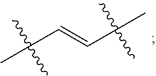 [SMILES: */C=C/*]

for each independent instance of Rₐ₂ and R_{b2}, Rₐ₂ and R_{b2} (1) independently are H or CH₃, or (2) Rₐ₂ and R_{b2} are

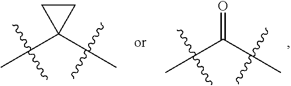

or (3) two of CRₐ₂R_{b2} are

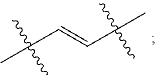 [SMILES: */C=C/*]

for each independent instance of R_{c1} and R_{d1}, R_{c1} and R_{d1} (1) independently are H or CH₃, or (2) R_{c1} and R_{d1} are

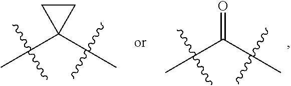

or (3) two of CR_{c1}R_{d1} are

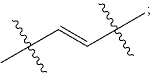 [SMILES: */C=C/*]

for each independent instance of R_{c2} and R_{d2}, R_{c1} and R_{d2} (1) independently are H or CH₃, or (2) R_{c1} and R_{d2} are

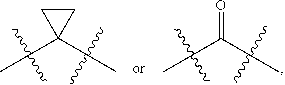

or (3) two of CR_{c2}R_{d2} are

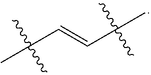 [SMILES: */C=C/*]

In some variations of formula (12b), each of D1 and D2 is a cationic dye moiety; ring A is aryl, heteroaryl, cycloalkyl, or heterocyclyl; n is 1-4; l₁ and l₂ are each 1; o₁ and o₂ independently are 1-8; Rₐ₁ and R_{b1} are

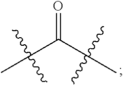 [SMILES: *C(*)=O]

Rₐ₂ and R_{b2} are

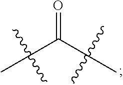 [SMILES: *C(*)=O]

for each independent instance of R_{c1} and R_{d1}, R_{c1} and R_{d1} (1) independently are H or CH₃, or (2) R_{c1} and R_{d1} are

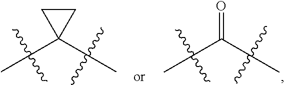

or (3) two of CR_{c1}R_{d1} are

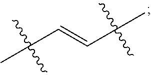 [SMILES: */C=C/*]

for each independent instance of R_{c2} and R_{d2}, R_{c1} and R_{d2} (1) independently are H or CH₃, or (2) R_{c2} and R_{d2} are

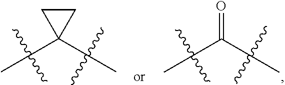

or (3) two of CR_{c2}R_{d2} are

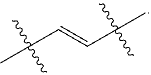 [SMILES: */C=C/*]

In some variations of formula (12b), n is 2; l₁ and l₂ are each 1; Rₐ₁ and R_{b1} are

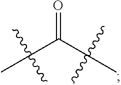 [SMILES: *C(*)=O]

Rₐ₂ and R_{b2} are

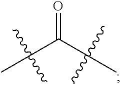 [SMILES: *C(*)=O]

and each ring A independently is aryl, heteroaryl, cycloalkyl, or heterocyclyl, wherein the rings are joined together to form one of the regioisomeric moieties shown, where each ring A is exemplified as a phenyl ring:

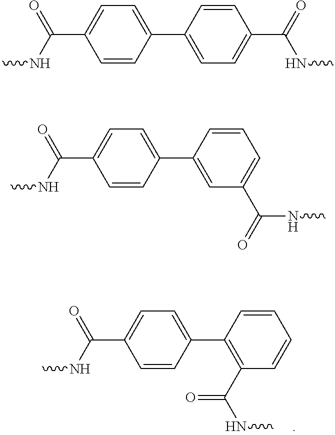

In other similar variations of formula (12b), n is 3 or 4, each ring A independently is aryl, heteroaryl, cycloalkyl, or heterocyclyl and are linked together to form extended regioisomeric chains. In particular variations, ring A is aryl or heteroaryl.

In some of the variations of formula (12b) described above, D1 and D2 are safranin-O moieties, as shown in formula (12c):

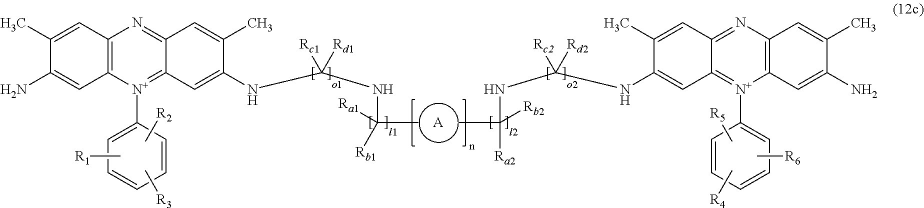

in which l₁, l₂, n, o₁, o₂, ring A, Rₐ₁, R_{b1}, Rₐ₂, R_{b2}, R_{c1}, R_{d1}, R_{c2} and R_{d2} are as described for formula (12b), R₁, R₂, R₃, R₄, R₅, and R₆ independently are absent or independently are selected from —NH₂, —NHR, —NR₂, —OH, —O⁻, —NHCOCH₃, —NHCOR, —OCH₃, —OR, —C₂H₅, —R, —C₆H₅, —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR), and R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some of the variations of formula (12c), each o₁ and o₂ is 1-8, each R_{c1}, R_{d1}, R_{c2} and R_{d2} is H, and R₁ to R₆ are as described above for formula (12b). In some of these variations, o₁ and o₂ are 6 or 8, each Rₐ and R_{b} is H, and each of R₁ to R₆ independently is absent or is a halo. In particular variations, o₁ and o₂ are 6 or 8, each Rₐ and R_{b} is H, and R₁ to R₆ are all absent.

In some of the variations of formula (12c), each o₁ and o₂ is 1-8, each R_{c1}, R_{d1}, R_{c2} and R_{d2} are either H or

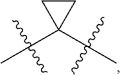 [SMILES: *C1(*)CC1]

and R₁ to R₆ are as described above for formula (12b). In some of these variations, o₁ and o₂ are 6 or 8, Rₐ and R_{b} are either H or

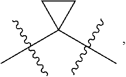 [SMILES: *C1(*)CC1]

and each of R₁ to R₆ independently is absent or is a halo. In particular variations, o₁ and o₂ are 6 or 8, Rₐ and R_{b} are either H or

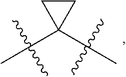 [SMILES: *C1(*)CC1]

and R₁ to R₆ are all absent.

In some of the variations of formula (12c), each o₁ and o₂ is 1-8, each R_{c1}, R_{d1}, R_{c2} and R_{d2} are either H or

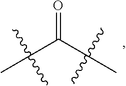 [SMILES: *C(*)=O]

and R₁ to R₆ are as described above for formula (12b). In some of these variations, o₁ and o₂ are 6 or 8, Rₐ and R_{b} are either H or

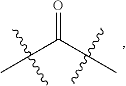 [SMILES: *C(*)=O]

and each of R₁ to R₆ independently is absent or is a halo. In particular variations, o₁ and o₂ are 6 or 8, Rₐ and R_{b} are either H or

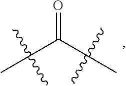 [SMILES: *C(*)=O]

and R₁ to R₆ are all absent.

In some of the variations of formula (12c), each o₁ and o₂ is 1-8, each R_{c1}, R_{d1}, R_{c2} and R_{d2} are either H or two of CR_{c1}R_{d1} and CR_{c2}R_{d2} are

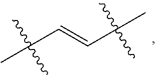 [SMILES: */C=C/*]

and R₁ to R₆ are as described above for formula (12b). In some of these variations, o₁ and o₂ are 6 or 8, each R_{c1}, R_{d1}, R_{c2} and R_{d2} are either H or two of CR_{c1}R_{d1} and CR_{c2}R_{d2} are

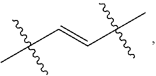 [SMILES: */C=C/*]

and each of R₁ to R₆ independently is absent or is a halo. In particular variations, o₁ and o₂ are 6 or 8, each R_{c1}, R_{d1}, R_{c2} and R_{d2} are either H or two of CR_{c1}R_{d1} and CR_{c2}R_{d2} are

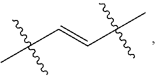 [SMILES: */C=C/*]

and R₁ to R₆ are all absent.

One of skill in the art can readily visualize and prepare other cationic multimers falling within formulae (12) or (12b) in which other cationic dye moieties are used in place of one or both of the safranin-O moieties.

Some cationic dye dimers fall within formula (13):

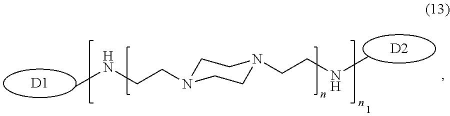

wherein each of D1 and D2 is a cationic dye moiety and n is 0-6, and n₁ is 1-4.

In some variations of formula (13), D1 and D2 are different cationic dye moieties. In other variations of formula (13), D1 and D2 are the same cationic dye moiety. In some variations of formula (13), D1 and D2 are independently selected from the group consisting of safranin-O, toluidine blue, azure A, azure B, azure C, acridine orange, acriflavine, and methylene blue.

In some variations of formula (13) described in the paragraphs above, n is 0-6, 0-5, 0-4, 0-3, 0-2, 0-1, 1-6, 1-5, 1-4, 1-3, 1-2, 2-6, 2-5, 2-4, 2-3, 3-6, 3-5, 3-4, 4-6, 4-5, 5-6, 0, 1, 2, 3, 4, 5, or 6.

In some variations of formula (13) described in the paragraphs above, n₁ is 1-4, 1-3, 1-2, 2-4, 2-3, 3-4, 1, 2, 3, or 4.

In some variations of formula (13) described in the paragraphs above, D1 is safranin-O. In some variations of formula (13) described in the paragraphs above, D2 is safranin-O. In some variations of formula (13) described in the paragraphs above, D1 and D2 are safranin-O.

In some variations of formula (13) described in the paragraph above, the pendant phenyl ring of D1 is unsubstituted. In some variations of formula (13) described in the paragraph above, the pendant phenyl ring of D1 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D1 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D1 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (13) described in the paragraph above, the pendant phenyl ring of D2 is unsubstituted. In some variations of formula (13) described in the paragraph above, the pendant phenyl ring of D2 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D2 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D2 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

### Examples of Cationic Dye Trimers and Other Multimers

Some cationic dye trimers fall within formula (14):

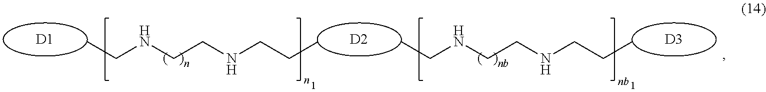

in which each of D1, D2, and D3 is a cationic dye moiety, n is 0-6, n₁ is 1-4, n_{b} is 0-6, and n_{b1} is 0-6.

In some variations of formula (14), D1, D2, and D3 are different cationic dye moieties. In some variations of formula (14), D1, D2, and D3 are the same cationic dye moiety. In other variations of formula (14), D1 and D2 are the same cationic dye moiety and D3 is a different cationic dye moiety. In other variations of formula (14), D1 and D3 are the same cationic dye moiety and D2 is a different cationic dye moiety. In other variations of formula (14), D2 and D3 are the same cationic dye moiety and D1 is a different cationic dye moiety. In some variations of formula (14), each of D1, D2, and D3 independently is selected from the group consisting of safranin-O, toluidine blue, azure A, azure B, azure C, acridine orange, acriflavine, and methylene blue.

In some variations of formula (14) described in the paragraphs above, n is 0-6, 0-5, 0-4, 0-3, 0-2, 0-1, 1-6, 1-5, 1-4, 1-3, 1-2, 2-6, 2-5, 2-4, 2-3, 3-6, 3-5, 3-4, 4-6, 4-5, 5-6, 0, 1, 2, 3, 4, 5, or 6.

In some variations of formula (14) described in the paragraphs above, n₁ is 1-4, 1-3, 1-2, 2-4, 2-3, 3-4, 1, 2, 3, or 4.

In some variations of formula (14) described in the paragraphs above, nb is 0-6, 0-5, 0-4, 0-3, 0-2, 0-1, 1-6, 1-5, 1-4, 1-3, 1-2, 2-6, 2-5, 2-4, 2-3, 3-6, 3-5, 3-4, 4-6, 4-5, 5-6, 0, 1, 2, 3, 4, 5, or 6.

In some variations of formula (14) described in the paragraphs above, n_{b1} is 1-4, 1-3, 1-2, 2-4, 2-3, 3-4, 1, 2, 3, or 4.

In some variations of formula (14) described in the paragraphs above, D1 is safranin-O. In some variations of formula (14) described in the paragraphs above, D2 is safranin-O. In some variations of formula (14) described in the paragraphs above, D3 is safranin-O. In some variations of formula (14) described in the paragraphs above, D1 and D2 are safranin-O. In some variations of formula (14) described in the paragraphs above, D1 and D3 are safranin-O. In some variations of formula (14) described in the paragraphs above, D2 and D3 are safranin-O.

In some variations of formula (14) described in the paragraph above, the pendant phenyl ring of D1 is unsubstituted. In some variations of formula (14) described in the paragraph above, the pendant phenyl ring of D1 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D1 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D1 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (14) described in the two paragraphs above, the pendant phenyl ring of D2 is unsubstituted. In some variations of formula (14) described in the paragraph above, the pendant phenyl ring of D2 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D2 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D2 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (14) described in the three paragraphs above, the pendant phenyl ring of D3 is unsubstituted. In some variations of formula (14) described in the paragraph above, the pendant phenyl ring of D3 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D3 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D3 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

Some cationic dye trimers fall within formula (15):

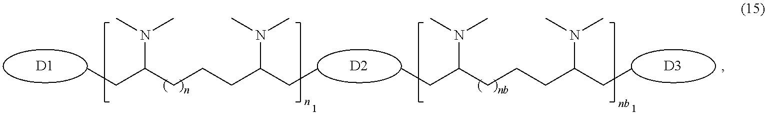

in which each of D1, D2, and D3 is a cationic dye moiety, n and nb independently are 0-6, and n₁ and n_{b1} independently are 1-4.

In some variations of formula (15), D1, D2, and D3 are different cationic dye moieties. In some variations of formula (15), D1, D2, and D3 are the same cationic dye moiety. In other variations of formula (15), D1 and D2 are the same cationic dye moiety and D3 is a different cationic dye moiety. In other variations of formula (15), D1 and D3 are the same cationic dye moiety and D2 is a different cationic dye moiety. In other variations of formula (15), D2 and D3 are the same cationic dye moiety and D1 is a different cationic dye moiety. In some variations of formula (15), each of D1, D2, and D3 independently is selected from the group consisting of safranin-O, toluidine blue, azure A, azure B, azure C, acridine orange, acriflavine, and methylene blue.

In some variations of formula (15) described in the paragraphs above, n is n is 0-6, 0-5, 0-4, 0-3, 0-2, 0-1, 1-6, 1-5, 1-4, 1-3, 1-2, 2-6, 2-5, 2-4, 2-3, 3-6, 3-5, 3-4, 4-6, 4-5, 5-6, 0, 1, 2, 3, 4, 5, or 6.

In some variations of formula (15) described in the paragraphs above, n₁ is 1-4, 1-3, 1-2, 2-4, 2-3, 3-4, 1, 2, 3, or 4.

In some variations of formula (15) described in the paragraphs above, nb is 0-6, 0-5, 0-4, 0-3, 0-2, 0-1, 1-6, 1-5, 1-4, 1-3, 1-2, 2-6, 2-5, 2-4, 2-3, 3-6, 3-5, 3-4, 4-6, 4-5, 5-6, 0, 1, 2, 3, 4, 5, or 6.

In some variations of formula (15) described in the paragraphs above, n_{b1} is 1-4, 1-3, 1-2, 2-4, 2-3, 3-4, 1, 2, 3, or 4.

In some variations of formula (15) described in the paragraphs above, D1 is safranin-O. In some variations of formula (15) described in the paragraphs above, D2 is safranin-O. In some variations of formula (15) described in the paragraphs above, D3 is safranin-O. In some variations of formula (15) described in the paragraphs above, D1 and D2 are safranin-O. In some variations of formula (15) described in the paragraphs above, D1 and D3 are safranin-O. In some variations of formula (15) described in the paragraphs above, D2 and D3 are safranin-O.

In some variations of formula (15) described in the paragraph above, the pendant phenyl ring of D1 is unsubstituted. In some variations of formula (15) described in the paragraph above, the pendant phenyl ring of D1 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D1 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D1 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (15) described in the two paragraphs above, the pendant phenyl ring of D2 is unsubstituted. In some variations of formula (15) described in the paragraph above, the pendant phenyl ring of D2 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D2 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D2 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (15) described in the three paragraphs above, the pendant phenyl ring of D3 is unsubstituted. In some variations of formula (15) described in the paragraph above, the pendant phenyl ring of D3 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D3 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D3 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

Some cationic dye trimers fall within formula (16):

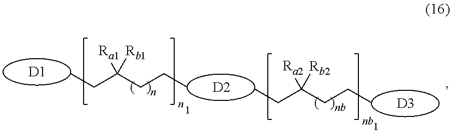

in which each of D1, D2, and D3 is a cationic dye moiety; n and nb independently are 0-6; n₁ and n₁ independently are 1-4; for each independent instance of Rₐ₁ and R_{b1}, Rₐ₁ and R_{b1} (1) independently are H or CH₃, or (2) Rₐ₁ and R_{b1} are

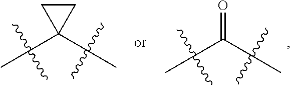

or (3) two of CRₐ₁R_{b1} are

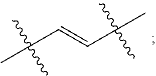 [SMILES: */C=C/*]

and, for each independent instance of Rₐ₂ and R_{b2}, Rₐ₂ and R_{b2} (1) independently are H or CH₃, or (2) Rₐ₂ and R_{b2} are

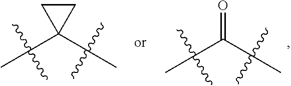

or (3) two of CRₐ₂R_{b2} are

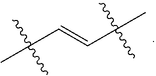 [SMILES: */C=C/*]

In some variations of formula (16), D1, D2, and D3 are different cationic dye moieties. In some variations of formula (16), D1, D2, and D3 are the same cationic dye moiety. In other variations of formula (16), D1 and D2 are the same cationic dye moiety and D3 is a different cationic dye moiety. In other variations of formula (16), D1 and D3 are the same cationic dye moiety and D2 is a different cationic dye moiety. In other variations of formula (16), D2 and D3 are the same cationic dye moiety and D1 is a different cationic dye moiety. In some variations of formula (16), each of D1, D2, and D3 independently is selected from the group consisting of safranin-O, toluidine blue, azure A, azure B, azure C, acridine orange, acriflavine, and methylene blue.

In some variations of formula (16) described in the paragraphs above, n is 0-6, 0-5, 0-4, 0-3, 0-2, 0-1, 1-6, 1-5, 1-4, 1-3, 1-2, 2-6, 2-5, 2-4, 2-3, 3-6, 3-5, 3-4, 4-6, 4-5, 5-6, 0, 1, 2, 3, 4, 5, or 6.

In some variations of formula (16) described in the paragraphs above, n₁ is 1-4, 1-3, 1-2, 2-4, 2-3, 3-4, 1, 2, 3, or 4.

In some variations of formula (16) described in the paragraphs above, nb is 0-6, 0-5, 0-4, 0-3, 0-2, 0-1, 1-6, 1-5, 1-4, 1-3, 1-2, 2-6, 2-5, 2-4, 2-3, 3-6, 3-5, 3-4, 4-6, 4-5, 5-6, 0, 1, 2, 3, 4, 5, or 6.

In some variations of formula (16) described in the paragraphs above, n_{b1} is 1-4, 1-3, 1-2, 2-4, 2-3, 3-4, 1, 2, 3, or 4.

In some variations of formula (16) described in the paragraphs above, Rₐ₁ is H and R_{b1} is H. In some variations of formula (16) described in the paragraphs above, Rₐ₁ is H and R_{b1} is CH₃. In some variations of formula (16) described in the paragraphs above, Rₐ₁ and R_{b1} are both CH₃. In some variations of formula (16) described in the paragraphs above, Rₐ₁ and R_{b1} are

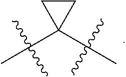 [SMILES: *C1(*)CC1]

In some variations of formula (16) described in the paragraphs above, Rₐ₁ and R_{b1} are

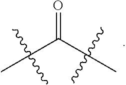 [SMILES: *C(*)=O]

In some variations of formula (16) described in the paragraphs above, two of CRₐ₁R_{b1} are

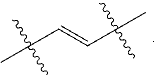 [SMILES: */C=C/*]

In some variations of formula (16) described in the paragraphs above, Rₐ₂ is H and R_{b2} is H. In some variations of formula (16) described in the paragraphs above, Rₐ₂ is H and R_{b2} is CH₃. In some variations of formula (16) described in the paragraphs above, Rₐ₂ and R_{b2} are both CH₃. In some variations of formula (16) described in the paragraphs above, Rₐ₂ and R_{b2} are

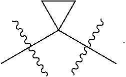 [SMILES: *C1(*)CC1]

In some variations of formula (16) described in the paragraphs above, Rₐ₂ and R_{b2} are

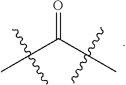 [SMILES: *C(*)=O]

In some variations of formula (16) described in the paragraphs above, two of CRₐ₂R_{b2} are

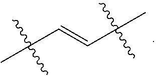 [SMILES: */C=C/*]

In some variations of formula (16) described in the paragraphs above, D1 is safranin-O.

In some variations of formula (16) described in the paragraphs above, D2 is safranin-O. In some variations of formula (16) described in the paragraphs above, D3 is safranin-O. In some variations of formula (16) described in the paragraphs above, D1 and D2 are safranin-O. In some variations of formula (16) described in the paragraphs above, D1 and D3 are safranin-O. In some variations of formula (16) described in the paragraphs above, D2 and D3 are safranin-O.

In some variations of formula (16) described in the paragraph above, the pendant phenyl ring of D1 is unsubstituted. In some variations of formula (16) described in the paragraph above, the pendant phenyl ring of D1 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D1 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D1 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (16) described in the two paragraphs above, the pendant phenyl ring of D2 is unsubstituted. In some variations of formula (16) described in the paragraph above, the pendant phenyl ring of D2 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D2 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D2 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (16) described in the three paragraphs above, the pendant phenyl ring of D3 is unsubstituted. In some variations of formula (16) described in the paragraph above, the pendant phenyl ring of D3 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D3 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D3 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

Some cationic dye trimers fall within formula (17):

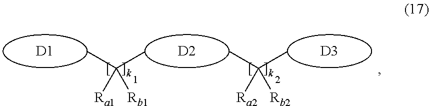

in which each of D1, D2, and D3 is a cationic dye moiety; k₁ is 2-10; k₂ is 2-10; for each independent instance of Rₐ₁ and R_{b1}, Rₐ₁ and R_{b1} (1) independently are H or CH₃, or (2) Rₐ₁ and R_{b1} are

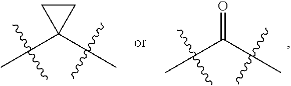

or (3) two of CRₐ₁R_{b1} are

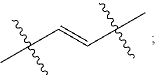 [SMILES: */C=C/*]

and, for each independent instance of Rₐ₂ and R_{b2}, Rₐ₂ and R_{b2} (1) independently are H or CH₃, or (2) Rₐ₂ and R_{b2} are

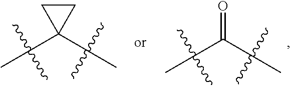

or (3) two of CRₐ₂R_{b2} are

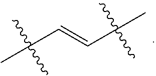 [SMILES: */C=C/*]

In some variations of formula (17), D1, D2, and D3 are different cationic dye moieties. In some variations of formula (17), D1, D2, and D3 are the same cationic dye moiety. In other variations of formula (17), D1 and D2 are the same cationic dye moiety and D3 is a different cationic dye moiety. In other variations of formula (17), D1 and D3 are the same cationic dye moiety and D2 is a different cationic dye moiety. In other variations of formula (17), D2 and D3 are the same cationic dye moiety and D1 is a different cationic dye moiety. In some variations of formula (17), each of D1, D2, and D3 independently is selected from the group consisting of safranin-O, toluidine blue, azure A, azure B, azure C, acridine orange, acriflavine, and methylene blue.

In some variations of formula (17) described in the paragraphs above, k₁ is 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, 9-10, 2, 3, 4, 5, 6, 7, 8, 9, or 10.

In some variations of formula (17) described in the paragraphs above, k₂ is 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, 9-10, 2, 3, 4, 5, 6, 7, 8, 9, or 10.

In some variations of formula (17) described in the paragraphs above, each Rₐ₁ is H and each R_{b1} is H. In some variations of formula (17) described in the paragraphs above, each Rₐ₁ is H and each R_{b1} is CH₃. In some variations of formula (17) described in the paragraphs above, each Rₐ₁ and R_{b1} is

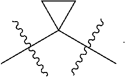 [SMILES: *C1(*)CC1]

In some variations of formula (17) described in the paragraphs above, each Rₐ₁ and R_{b1} is

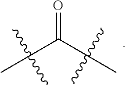 [SMILES: *C(*)=O]

In some variations of formula (17) described in the paragraphs above, each two of CRₐ₁R_{b1} are

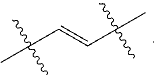 [SMILES: */C=C/*]

In some variations of formula (17) described in the paragraphs above, in a first occurrence of Rₐ₁ and R_{b1}, each of Rₐ₁ and R_{b1} is H, and the remaining occurrences of Rₐ₁ and R_{b1} are as defined above for formula (17). In some variations of formula (17) described in the paragraphs above, in a first occurrence of Rₐ₁ and R_{b1}, each of Rₐ₁ and R_{b1} is CH₃, and the remaining occurrences of Rₐ₁ and R_{b1} are as defined above for formula (17). In some variations of formula (17) described in the paragraphs above, in a first occurrence of Rₐ₁ and R_{b1}, a first Rₐ₁ is H, a first R_{b1} is CH₃, and the remaining occurrences of Rₐ₁ and R_{b1} are as defined above for formula (17). In some variations of formula (17) described in the paragraphs above, in a first occurrence of Rₐ₁ and R_{b1}, Rₐ₁ and R_{b1} are

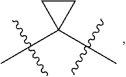 [SMILES: *C1(*)CC1]

and the remaining occurrences of Rₐ₁ and R_{b1} are as defined above for formula (17). In some variations of formula (17) described in the paragraphs above, in a first occurrence of Rₐ₁ and R_{b1}, Rₐ₁ and R_{b1} are

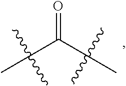 [SMILES: *C(*)=O]

and the remaining occurrences of Rₐ₁ and R_{b1} are as defined above for formula (17).

In some variations of formula (17) described in the paragraphs above, in two occurrences of Rₐ₁ and R_{b1}, each of Rₐ₁ and R_{b1} is H, and the remaining occurrences of Rₐ₁ and R_{b1} are as defined above for formula (17). In some variations of formula (17) described in the paragraphs above, in two occurrences of Rₐ₁ and R_{b1}, each of Rₐ₁ and R_{b1} is CH₃, and the remaining occurrences of Rₐ₁ and R_{b1} are as defined above for formula (17). In some variations of formula (17) described in the paragraphs above, in two occurrences of Rₐ₁ and R_{b1}, two of Rₐ₁ are H, two of R_{b1} are CH₃, and the remaining occurrences of Rₐ₁ and R_{b1} are as defined above for formula (17). In some variations of formula (17) described in the paragraphs above, in two occurrences of Rₐ₁ and R_{b1}, Rₐ₁ and R_{b1} are

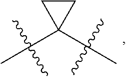 [SMILES: *C1(*)CC1]

and the remaining occurrences of Rₐ₁ and R_{b1} are as defined above for formula (17). In some variations of formula (17) described in the paragraphs above, in two occurrences of Rₐ₁ and R_{b1}, Rₐ₁ and R_{b1} are

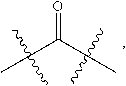 [SMILES: *C(*)=O]

and the remaining occurrences of Rₐ₁ and R_{b1} are as defined above for formula (17).

In some variations of formula (17) described in the paragraphs above, in three occurrences of Rₐ₁ and R_{b1}, each of Rₐ₁ and R_{b1} is H, and the remaining occurrences of Rₐ₁ and R_{b1} are as defined above for formula (17). In some variations of formula (17) described in the paragraphs above, in three occurrences of Rₐ₁ and R_{b1}, each of Rₐ₁ and R_{b1} is CH₃, and the remaining occurrences of Rₐ₁ and R_{b1} are as defined above for formula (17). In some variations of formula (17) described in the paragraphs above, in three occurrences of Rₐ₁ and R_{b1}, three of Rₐ₁ are H, three of R_{b1} are CH₃, and the remaining occurrences of Rₐ₁ and R_{b1} are as defined above for formula (17). In some variations of formula (17) described in the paragraphs above, in three occurrences of Rₐ₁ and R_{b1}, Rₐ₁ and R_{b1} are

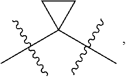 [SMILES: *C1(*)CC1]

and the remaining occurrences of Rₐ₁ and R_{b1} are as defined above for formula (17). In some variations of formula (17) described in the paragraphs above, in three occurrences of Rₐ₁ and R_{b1}, Rₐ₁ and R_{b1} are

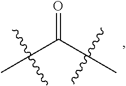 [SMILES: *C(*)=O]

and the remaining occurrences of Rₐ₁ and R_{b1} are as defined above for formula (17).

In some variations of formula (17) described in the paragraphs above, in four occurrences of Rₐ₁ and R_{b1}, each of Rₐ₁ and R_{b1} is H, and the remaining occurrences of Rₐ₁ and R_{b1} are as defined above for formula (17). In some variations of formula (17) described in the paragraphs above, in four occurrences of Rₐ₁ and R_{b1}, each of Rₐ₁ and R_{b1} is CH₃, and the remaining occurrences of Rₐ₁ and R_{b1} are as defined above for formula (17). In some variations of formula (17) described in the paragraphs above, in four occurrences of Rₐ₁ and R_{b1}, four of Rₐ₁ are H, four of R_{b1} are CH₃, and the remaining occurrences of Rₐ₁ and R_{b1} are as defined above for formula (17). In some variations of formula (17) described in the paragraphs above, in four occurrences of Rₐ₁ and R_{b1}, Rₐ₁ and R_{b1} are

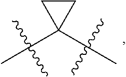 [SMILES: *C1(*)CC1]

and the remaining occurrences of Rₐ₁ and R_{b1} are as defined above for formula (17). In some variations of formula (17) described in the paragraphs above, in four occurrences of Rₐ₁ and R_{b1}, Rₐ₁ and R_{b1} are

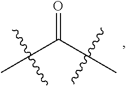 [SMILES: *C(*)=O]

and the remaining occurrences of Rₐ₁ and R_{b1} are as defined above for formula (17).

In some variations of formula (17) described in the paragraphs above, in five occurrences of Rₐ₁ and R_{b1}, each of Rₐ₁ and R_{b1} is H, and the remaining occurrences of Rₐ₁ and R_{b1} are as defined above for formula (17). In some variations of formula (17) described in the paragraphs above, in five occurrences of Rₐ₁ and R_{b1}, each of Rₐ₁ and R_{b1} is CH₃, and the remaining occurrences of Rₐ₁ and R_{b1} are as defined above for formula (17). In some variations of formula (17) described in the paragraphs above, in five occurrences of Rₐ₁ and R_{b1}, five of Rₐ₁ are H, five of R_{b1} are CH₃, and the remaining occurrences of Rₐ₁ and R_{b1} are as defined above for formula (17). In some variations of formula (17) described in the paragraphs above, in five occurrences of Rₐ₁ and R_{b1}, Rₐ₁ and R_{b1} are

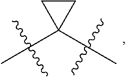 [SMILES: *C1(*)CC1]

and the remaining occurrences of Rₐ₁ and R_{b1} are as defined above for formula (17). In some variations of formula (17) described in the paragraphs above, in five occurrences of Rₐ₁ and R_{b1}, Rₐ₁ and R_{b1} are

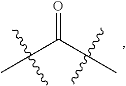 [SMILES: *C(*)=O]

and the remaining occurrences of Rₐ₁ and R_{b1} are as defined above for formula (17).

In some variations of formula (17) described in the paragraphs above, in six occurrences of Rₐ₁ and R_{b1}, each of Rₐ₁ and R_{b1} is H, and the remaining occurrences of Rₐ₁ and R_{b1} are as defined above for formula (17). In some variations of formula (17) described in the paragraphs above, in six occurrences of Rₐ₁ and R_{b1}, each of Rₐ₁ and R_{b1} is CH₃, and the remaining occurrences of Rₐ₁ and R_{b1} are as defined above for formula (17). In some variations of formula (17) described in the paragraphs above, in six occurrences of Rₐ₁ and R_{b1}, six of Rₐ₁ are H, six of R_{b1} are CH₃, and the remaining occurrences of Rₐ₁ and R_{b1} are as defined above for formula (17). In some variations of formula (17) described in the paragraphs above, in six occurrences of Rₐ₁ and R_{b1}, Rₐ₁ and R_{b1} are

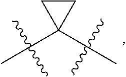 [SMILES: *C1(*)CC1]

and the remaining occurrences of Rₐ₁ and R_{b1} are as defined above for formula (17). In some variations of formula (17) described in the paragraphs above, in six occurrences of Rₐ₁ and R_{b1}, Rₐ₁ and R_{b1} are

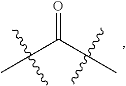 [SMILES: *C(*)=O]

and the remaining occurrences of Rₐ₁ and R_{b1} are as defined above for formula (17).

In some variations of formula (17) described in the paragraphs above, in seven occurrences of Rₐ₁ and R_{b1}, each of Rₐ₁ and R_{b1} is H, and the remaining occurrences of Rₐ₁ and R_{b1} are as defined above for formula (17). In some variations of formula (17) described in the paragraphs above, in seven occurrences of Rₐ₁ and R_{b1}, each of Rₐ₁ and R_{b1} is CH₃, and the remaining occurrences of Rₐ₁ and R_{b1} are as defined above for formula (17). In some variations of formula (17) described in the paragraphs above, in seven occurrences of Rₐ₁ and R_{b1}, seven of Rₐ₁ are H, seven of R_{b1} are CH₃, and the remaining occurrences of Rₐ₁ and R_{b1} are as defined above for formula (17). In some variations of formula (17) described in the paragraphs above, in seven occurrences of Rₐ₁ and R_{b1}, Rₐ₁ and R_{b1} are

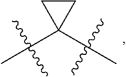 [SMILES: *C1(*)CC1]

and the remaining occurrences of Rₐ₁ and R_{b1} are as defined above for formula (17). In some variations of formula (17) described in the paragraphs above, in seven occurrences of Rₐ₁ and R_{b1}, Rₐ₁ and R_{b1} are

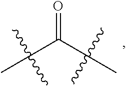 [SMILES: *C(*)=O]

and the remaining occurrences of Rₐ₁ and R_{b1} are as defined above for formula (17).

In some variations of formula (17) described in the paragraphs above, in eight occurrences of Rₐ₁ and R_{b1}, each of Rₐ₁ and R_{b1} is H, and the remaining occurrences of Rₐ₁ and R_{b1} are as defined above for formula (17). In some variations of formula (17) described in the paragraphs above, in eight occurrences of Rₐ₁ and R_{b1}, each of Rₐ₁ and R_{b1} is CH₃, and the remaining occurrences of Rₐ₁ and R_{b1} are as defined above for formula (17). In some variations of formula (17) described in the paragraphs above, in eight occurrences of Rₐ₁ and R_{b1}, eight of Rₐ₁ are H, eight of R_{b1} are CH₃, and the remaining occurrences of Rₐ₁ and R_{b1} are as defined above for formula (17). In some variations of formula (17) described in the paragraphs above, in eight occurrences of Rₐ₁ and R_{b1}, Rₐ₁ and R_{b1} are

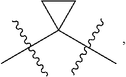 [SMILES: *C1(*)CC1]

and the remaining occurrences of Rₐ₁ and R_{b1} are as defined above for formula (17). In some variations of formula (17) described in the paragraphs above, in eight occurrences of Rₐ₁ and R_{b1}, Rₐ₁ and R_{b1} are

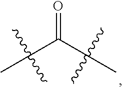 [SMILES: *C(*)=O]

and the remaining occurrences of Rₐ₁ and R_{b1} are as defined above for formula (17).

In some variations of formula (17) described in the paragraphs above, in nine occurrences of Rₐ₁ and R_{b1}, each of Rₐ₁ and R_{b1} is H, and the remaining occurrences of Rₐ₁ and R_{b1} are as defined above for formula (17). In some variations of formula (17) described in the paragraphs above, in nine occurrences of Rₐ₁ and R_{b1}, each of Rₐ₁ and R_{b1} is CH₃, and the remaining occurrences of Rₐ₁ and R_{b1} are as defined above for formula (17). In some variations of formula (17) described in the paragraphs above, in nine occurrences of Rₐ₁ and R_{b1}, nine of Rₐ₁ are H, nine of R_{b1} are CH₃, and the remaining occurrences of Rₐ₁ and R_{b1} are as defined above for formula (17). In some variations of formula (17) described in the paragraphs above, in nine occurrences of Rₐ₁ and R_{b1}, Rₐ₁ and R_{b1} are

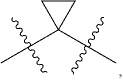 [SMILES: *C1(*)CC1]

and the remaining occurrences of Rₐ₁ and R_{b1} are as defined above for formula (17). In some variations of formula (17) described in the paragraphs above, in nine occurrences of Rₐ₁ and R_{b1}, Rₐ₁ and R_{b1} are

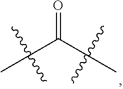 [SMILES: *C(*)=O]

and the remaining occurrences of Rₐ₁ and R_{b1} are as defined above for formula (17).

In some variations of formula (17) described in the paragraphs above, in ten occurrences of Rₐ₁ and R_{b1}, each of Rₐ₁ and R_{b1} is H, and the remaining occurrences of Rₐ₁ and R_{b1} are as defined above for formula (17). In some variations of formula (17) described in the paragraphs above, in ten occurrences of Rₐ₁ and R_{b1}, each of Rₐ₁ and R_{b1} is CH₃, and the remaining occurrences of Rₐ₁ and R_{b1} are as defined above for formula (17). In some variations of formula (17) described in the paragraphs above, in ten occurrences of Rₐ₁ and R_{b1}, ten of Rₐ₁ are H, ten of R_{b1} are CH₃, and the remaining occurrences of Rₐ₁ and R_{b1} are as defined above for formula (17). In some variations of formula (17) described in the paragraphs above, in ten occurrences of Rₐ₁ and R_{b1}, Rₐ₁ and R_{b1} are

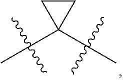 [SMILES: *C1(*)CC1]

and the remaining occurrences of Rₐ₁ and R_{b1} are as defined above for formula (17). In some variations of formula (17) described in the paragraphs above, in ten occurrences of Rₐ₁ and R_{b1}, Rₐ₁ and R_{b1} are

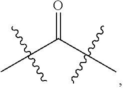 [SMILES: *C(*)=O]

and the remaining occurrences of Rₐ₁ and R_{b1} are as defined above for formula (17).

In some variations of formula (17) described in the paragraphs above, two occurrences of Rₐ₁ and R_{b1} are

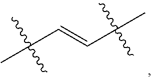 [SMILES: */C=C/*]

and the remaining occurrences of Rₐ₁ and R_{b1} are as defined above for formula (17). In some variations of formula (17) described in the paragraphs above, four occurrences of Rₐ₁ and R_{b1} are

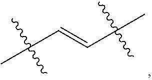 [SMILES: */C=C/*]

and the remaining occurrences of Rₐ₁ and R_{b1} are as defined above for formula (17). In some variations of formula (17) described in the paragraphs above, six occurrences of Rₐ₁ and R_{b1} are

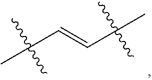 [SMILES: */C=C/*]

and the remaining occurrences of Rₐ₁ and R_{b1} are as defined above for formula (17). In some variations of formula (17) described in the paragraphs above, eight occurrences of Rₐ₁ and R_{b1} are

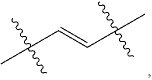 [SMILES: */C=C/*]

and the remaining occurrences of Rₐ₁ and R_{b1} are as defined above for formula (17). In some variations of formula (17) described in the paragraphs above, five occurrences of Rₐ₁ and R_{b1} are

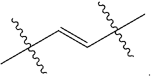 [SMILES: */C=C/*]

In some variations of formula (17) described in the paragraphs above, each Rₐ₂ is H and each R_{b2} is H. In some variations of formula (17) described in the paragraphs above, each Rₐ₂ is H and each R_{b2} is CH₃. In some variations of formula (17) described in the paragraphs above, each Rₐ₂ and R_{b2} is

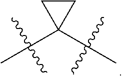 [SMILES: *C1(*)CC1]

In some variations of formula (17) described in the paragraphs above, each Rₐ₂ and R_{b2} is

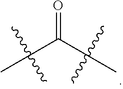 [SMILES: *C(*)=O]

In some variations of formula (17) described in the paragraphs above, each two of CRₐ₂R_{b2} are

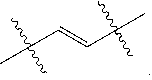 [SMILES: */C=C/*]

In some variations of formula (17) described in the paragraphs above, in a first occurrence of Rₐ₂ and R_{b2}, each of Rₐ₂ and R_{b2} is H, and the remaining occurrences of Rₐ₂ and R_{b2} are as defined above for formula (17). In some variations of formula (17) described in the paragraphs above, in a first occurrence of Rₐ₂ and R_{b2}, each of Rₐ₂ and R_{b2} is CH₃, and the remaining occurrences of Rₐ₂ and R_{b2} are as defined above for formula (17). In some variations of formula (17) described in the paragraphs above, in a first occurrence of Rₐ₂ and R_{b2}, a first Rₐ₂ is H, a first R_{b2} is CH₃, and the remaining occurrences of Rₐ₂ and R_{b2} are as defined above for formula (17). In some variations of formula (17) described in the paragraphs above, in a first occurrence of Rₐ₂ and R_{b2}, Rₐ₂ and R_{b2} are

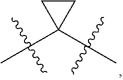 [SMILES: *C1(*)CC1]

and the remaining occurrences of Rₐ₂ and R_{b2} are as defined above for formula (17). In some variations of formula (17) described in the paragraphs above, in a first occurrence of Rₐ₂ and R_{b2}, Rₐ₂ and R_{b2} are

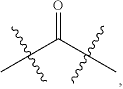 [SMILES: *C(*)=O]

and the remaining occurrences of Rₐ₂ and R_{b2} are as defined above for formula (17).

In some variations of formula (17) described in the paragraphs above, in two occurrences of Rₐ₂ and R_{b2}, each of Rₐ₂ and R_{b2} is H, and the remaining occurrences of Rₐ₂ and R_{b2} are as defined above for formula (17). In some variations of formula (17) described in the paragraphs above, in two occurrences of Rₐ₂ and R_{b2}, each of Rₐ₂ and R_{b2} is CH₃, and the remaining occurrences of Rₐ₂ and R_{b2} are as defined above for formula (17). In some variations of formula (17) described in the paragraphs above, in two occurrences of Rₐ₂ and R_{b2}, two of Rₐ₂ are H, two of R_{b2} are CH₃, and the remaining occurrences of Rₐ₂ and R_{b2} are as defined above for formula (17). In some variations of formula (17) described in the paragraphs above, in two occurrences of Rₐ₂ and R_{b2}, Rₐ₂ and R_{b2} are

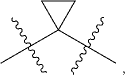 [SMILES: *C1(*)CC1]

and the remaining occurrences of Rₐ₂ and R_{b2} are as defined above for formula (17). In some variations of formula (17) described in the paragraphs above, in two occurrences of Rₐ₂ and R_{b2}, Rₐ₂ and R_{b2} are

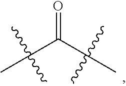 [SMILES: *C(*)=O]

and the remaining occurrences of Rₐ₂ and R_{b2} are as defined above for formula (17).

In some variations of formula (17) described in the paragraphs above, in three occurrences of Rₐ₂ and R_{b2}, each of Rₐ₂ and R_{b2} is H, and the remaining occurrences of Rₐ₂ and R_{b2} are as defined above for formula (17). In some variations of formula (17) described in the paragraphs above, in three occurrences of Rₐ₂ and R_{b2}, each of Rₐ₂ and R_{b2} is CH₃, and the remaining occurrences of Rₐ₂ and R_{b2} are as defined above for formula (17). In some variations of formula (17) described in the paragraphs above, in three occurrences of Rₐ₂ and R_{b2}, three of Rₐ₂ are H, three of R_{b2} are CH₃, and the remaining occurrences of Rₐ₂ and R_{b2} are as defined above for formula (17). In some variations of formula (17) described in the paragraphs above, in three occurrences of Rₐ₂ and R_{b2}, Rₐ₂ and R_{b2} are

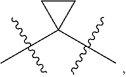 [SMILES: *C1(*)CC1]

and the remaining occurrences of Rₐ₂ and R_{b2} are as defined above for formula (17). In some variations of formula (17) described in the paragraphs above, in three occurrences of Rₐ₂ and R_{b2}, Rₐ₂ and R_{b2} are

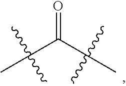 [SMILES: *C(*)=O]

and the remaining occurrences of Rₐ₂ and R_{b2} are as defined above for formula (17).

In some variations of formula (17) described in the paragraphs above, in four occurrences of Rₐ₂ and R_{b2}, each of Rₐ₂ and R_{b2} is H, and the remaining occurrences of Rₐ₂ and R_{b2} are as defined above for formula (17). In some variations of formula (17) described in the paragraphs above, in four occurrences of Rₐ₂ and R_{b2}, each of Rₐ₂ and R_{b2} is CH₃, and the remaining occurrences of Rₐ₂ and R_{b2} are as defined above for formula (17). In some variations of formula (17) described in the paragraphs above, in four occurrences of Rₐ₂ and R_{b2}, four of Rₐ₂ are H, four of R_{b2} are CH₃, and the remaining occurrences of Rₐ₂ and R_{b2} are as defined above for formula (17). In some variations of formula (17) described in the paragraphs above, in four occurrences of Rₐ₂ and R_{b2}, Rₐ₂ and R_{b2} are

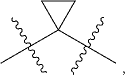 [SMILES: *C1(*)CC1]

and the remaining occurrences of Rₐ₂ and R_{b2} are as defined above for formula (17). In some variations of formula (17) described in the paragraphs above, in four occurrences of Rₐ₂ and R_{b2}, Rₐ₂ and R_{b2} are

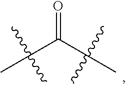 [SMILES: *C(*)=O]

and the remaining occurrences of Rₐ₂ and R_{b2} are as defined above for formula (17).

In some variations of formula (17) described in the paragraphs above, in five occurrences of Rₐ₂ and R_{b2}, each of Rₐ₂ and R_{b2} is H, and the remaining occurrences of Rₐ₂ and R_{b2} are as defined above for formula (17). In some variations of formula (17) described in the paragraphs above, in five occurrences of Rₐ₂ and R_{b2}, each of Rₐ₂ and R_{b2} is CH₃, and the remaining occurrences of Rₐ₂ and R_{b2} are as defined above for formula (17). In some variations of formula (17) described in the paragraphs above, in five occurrences of Rₐ₂ and R_{b2}, five of Rₐ₂ are H, five of R_{b2} are CH₃, and the remaining occurrences of Rₐ₂ and R_{b2} are as defined above for formula (17). In some variations of formula (17) described in the paragraphs above, in five occurrences of Rₐ₂ and R_{b2}, Rₐ₂ and R_{b2} are

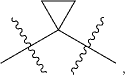 [SMILES: *C1(*)CC1]

and the remaining occurrences of Rₐ₂ and R_{b2} are as defined above for formula (17). In some variations of formula (17) described in the paragraphs above, in five occurrences of Rₐ₂ and R_{b2}, Rₐ₂ and R_{b2} are

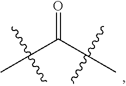 [SMILES: *C(*)=O]

and the remaining occurrences of Rₐ₂ and R_{b2} are as defined above for formula (17).

In some variations of formula (17) described in the paragraphs above, in six occurrences of Rₐ₂ and R_{b2}, each of Rₐ₂ and R_{b2} is H, and the remaining occurrences of Rₐ₂ and R_{b2} are as defined above for formula (17). In some variations of formula (17) described in the paragraphs above, in six occurrences of Rₐ₂ and R_{b2}, each of Rₐ₂ and R_{b2} is CH₃, and the remaining occurrences of Rₐ₂ and R_{b2} are as defined above for formula (17). In some variations of formula (17) described in the paragraphs above, in six occurrences of Rₐ₂ and R_{b2}, six of Rₐ₂ are H, six of R_{b2} are CH₃, and the remaining occurrences of Rₐ₂ and R_{b2} are as defined above for formula (17). In some variations of formula (17) described in the paragraphs above, in six occurrences of Rₐ₂ and R_{b2}, Rₐ₂ and R_{b2} are

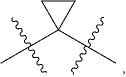 [SMILES: *C1(*)CC1]

and the remaining occurrences of Rₐ₂ and R_{b2} are as defined above for formula (17). In some variations of formula (17) described in the paragraphs above, in six occurrences of Rₐ₂ and R_{b2}, Rₐ₂ and R_{b2} are

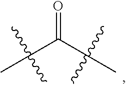 [SMILES: *C(*)=O]

and the remaining occurrences of Rₐ₂ and R_{b2} are as defined above for formula (17).

In some variations of formula (17) described in the paragraphs above, in seven occurrences of Rₐ₂ and R_{b2}, each of Rₐ₂ and R_{b2} is H, and the remaining occurrences of Rₐ₂ and R_{b2} are as defined above for formula (17). In some variations of formula (17) described in the paragraphs above, in seven occurrences of Rₐ₂ and R_{b2}, each of Rₐ₂ and R_{b2} is CH₃, and the remaining occurrences of Rₐ₂ and R_{b2} are as defined above for formula (17). In some variations of formula (17) described in the paragraphs above, in seven occurrences of Rₐ₂ and R_{b2}, seven of Rₐ₂ are H, seven of R_{b2} are CH₃, and the remaining occurrences of Rₐ₂ and R_{b2} are as defined above for formula (17). In some variations of formula (17) described in the paragraphs above, in seven occurrences of Rₐ₂ and R_{b2}, Rₐ₂ and R_{b2} are

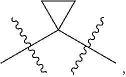 [SMILES: *C1(*)CC1]

and the remaining occurrences of Rₐ₂ and R_{b2} are as defined above for formula (17). In some variations of formula (17) described in the paragraphs above, in seven occurrences of Rₐ₂ and R_{b2}, Rₐ₂ and R_{b2} are

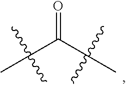 [SMILES: *C(*)=O]

and the remaining occurrences of Rₐ₂ and R_{b2} are as defined above for formula (17).

In some variations of formula (17) described in the paragraphs above, in eight occurrences of Rₐ₂ and R_{b2}, each of Rₐ₂ and R_{b2} is H, and the remaining occurrences of Rₐ₂ and R_{b2} are as defined above for formula (17). In some variations of formula (17) described in the paragraphs above, in eight occurrences of Rₐ₂ and R_{b2}, each of Rₐ₂ and R_{b2} is CH₃, and the remaining occurrences of Rₐ₂ and R_{b2} are as defined above for formula (17). In some variations of formula (17) described in the paragraphs above, in eight occurrences of Rₐ₂ and R_{b2}, eight of Rₐ₂ are H, eight of R_{b2} are CH₃, and the remaining occurrences of Rₐ₂ and R_{b2} are as defined above for formula (17). In some variations of formula (17) described in the paragraphs above, in eight occurrences of Rₐ₂ and R_{b2}, Rₐ₂ and R_{b2} are

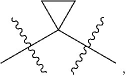 [SMILES: *C1(*)CC1]

and the remaining occurrences of Rₐ₂ and R_{b2} are as defined above for formula (17). In some variations of formula (17) described in the paragraphs above, in eight occurrences of Rₐ₂ and R_{b2}, Rₐ₂ and R_{b2} are

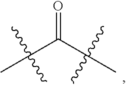 [SMILES: *C(*)=O]

and the remaining occurrences of Rₐ₂ and R_{b2} are as defined above for formula (17).

In some variations of formula (17) described in the paragraphs above, in nine occurrences of Rₐ₂ and R_{b2}, each of Rₐ₂ and R_{b2} is H, and the remaining occurrences of Rₐ₂ and R_{b2} are as defined above for formula (17). In some variations of formula (17) described in the paragraphs above, in nine occurrences of Rₐ₂ and R_{b2}, each of Rₐ₂ and R_{b2} is CH₃, and the remaining occurrences of Rₐ₂ and R_{b2} are as defined above for formula (17). In some variations of formula (17) described in the paragraphs above, in nine occurrences of Rₐ₂ and R_{b2}, nine of Rₐ₂ are H, nine of R_{b2} are CH₃, and the remaining occurrences of Rₐ₂ and R_{b2} are as defined above for formula (17). In some variations of formula (17) described in the paragraphs above, in nine occurrences of Rₐ₂ and R_{b2}, Rₐ₂ and R_{b2} are

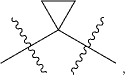 [SMILES: *C1(*)CC1]

and the remaining occurrences of Rₐ₂ and R_{b2} are as defined above for formula (17). In some variations of formula (17) described in the paragraphs above, in nine occurrences of Rₐ₂ and R_{b2}, Rₐ₂ and R_{b2} are

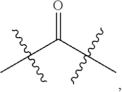 [SMILES: *C(*)=O]

and the remaining occurrences of Rₐ₂ and R_{b2} are as defined above for formula (17).

In some variations of formula (17) described in the paragraphs above, in ten occurrences of Rₐ₂ and R_{b2}, each of Rₐ₂ and R_{b2} is H, and the remaining occurrences of Rₐ₂ and R_{b2} are as defined above for formula (17). In some variations of formula (17) described in the paragraphs above, in ten occurrences of Rₐ₂ and R_{b2}, each of Rₐ₂ and R_{b2} is CH₃, and the remaining occurrences of Rₐ₂ and R_{b2} are as defined above for formula (17). In some variations of formula (17) described in the paragraphs above, in ten occurrences of Rₐ₂ and R_{b2}, ten of Rₐ₂ are H, ten of R_{b2} are CH₃, and the remaining occurrences of Rₐ₂ and R_{b2} are as defined above for formula (17). In some variations of formula (17) described in the paragraphs above, in ten occurrences of Rₐ₂ and R_{b2}, Rₐ₂ and R_{b2} are

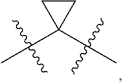 [SMILES: *C1(*)CC1]

and the remaining occurrences of Rₐ₂ and R_{b2} are as defined above for formula (17). In some variations of formula (17) described in the paragraphs above, in ten occurrences of Rₐ₂ and R_{b2}, Rₐ₂ and R_{b2} are

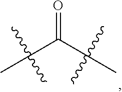 [SMILES: *C(*)=O]

and the remaining occurrences of Rₐ₂ and R_{b2} are as defined above for formula (17).

In some variations of formula (17) described in the paragraphs above, two occurrences of Rₐ₂ and R_{b2} are

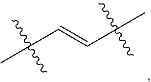 [SMILES: */C=C/*]

end the remaining occurrences of Rₐ₂ and R_{b2} are as defined above for formula (17). In some variations of formula (17) described in the paragraphs above, four occurrences of Rₐ₂ and R_{b2} are

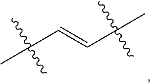 [SMILES: */C=C/*]

and the remaining occurrences of Rₐ₂ and R_{b2} are as defined above for formula (17). In some variations of formula (17) described in the paragraphs above, six occurrences of Rₐ₂ and R_{b2} are

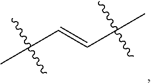 [SMILES: */C=C/*]

and the remaining occurrences of Rₐ₂ and R_{b2} are as defined above for formula (17). In some variations of formula (17) described in the paragraphs above, eight occurrences of Rₐ₂ and R_{b2} are

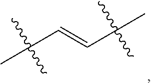 [SMILES: */C=C/*]

and the remaining occurrences of Rₐ₂ and R_{b2} are as defined above for formula (17). In some variations of formula (17) described in the paragraphs above, five occurrences of Rₐ₂ and R_{b2} are

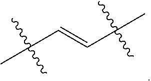 [SMILES: */C=C/*]

In some variations of formula (17) described in the paragraphs above, D1 is safranin-O. In some variations of formula (17) described in the paragraphs above, D2 is safranin-O. In some variations of formula (17) described in the paragraphs above, D3 is safranin-O. In some variations of formula (17) described in the paragraphs above, D1 and D2 are safranin-O. In some variations of formula (17) described in the paragraphs above, D1 and D3 are safranin-O. In some variations of formula (17) described in the paragraphs above, D2 and D3 are safranin-O.

In some variations of formula (17) described in the paragraph above, the pendant phenyl ring of D1 is unsubstituted. In some variations of formula (17) described in the paragraph above, the pendant phenyl ring of D1 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D1 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D1 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (17) described in the two paragraphs above, the pendant phenyl ring of D2 is unsubstituted. In some variations of formula (17) described in the paragraph above, the pendant phenyl ring of D2 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D2 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D2 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (17) described in the three paragraphs above, the pendant phenyl ring of D3 is unsubstituted. In some variations of formula (17) described in the paragraph above, the pendant phenyl ring of D3 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D3 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D3 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

Some cationic dye dimers fall within formula (18):

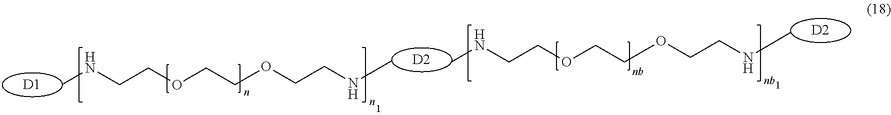

wherein each of D1, D2, and D3 is a cationic dye moiety, n and nb independently are 0-6, and n₁ and n_{b1} independently are 1-4.

In some variations of formula (18), D1, D2, and D3 are different cationic dye moieties. In some variations of formula (18), D1, D2, and D3 are the same cationic dye moiety. In other variations of formula (18), D1 and D2 are the same cationic dye moiety and D3 is a different cationic dye moiety. In other variations of formula (18), D1 and D3 are the same cationic dye moiety and D2 is a different cationic dye moiety. In other variations of formula (18), D2 and D3 are the same cationic dye moiety and D1 is a different cationic dye moiety. In some variations of formula (18), each of D1, D2, and D3 independently is selected from the group consisting of safranin-O, toluidine blue, azure A, azure B, azure C, acridine orange, acriflavine, and methylene blue.

In some variations of formula (18) described in the paragraphs above, n is 0-6, 0-5, 0-4, 0-3, 0-2, 0-1, 1-6, 1-5, 1-4, 1-3, 1-2, 2-6, 2-5, 2-4, 2-3, 3-6, 3-5, 3-4, 4-6, 4-5, 5-6, 0, 1, 2, 3, 4, 5, or 6.

In some variations of formula (18) described in the paragraphs above, nb is 0-6, 0-5, 0-4, 0-3, 0-2, 0-1, 1-6, 1-5, 1-4, 1-3, 1-2, 2-6, 2-5, 2-4, 2-3, 3-6, 3-5, 3-4, 4-6, 4-5, 5-6, 0, 1, 2, 3, 4, 5, or 6.

In some variations of formula (18) described in the paragraphs above, n₁ is 1-4, 1-3, 1-2, 2-4, 2-3, 3-4, 1, 2, 3, or 4.

In some variations of formula (18) described in the paragraphs above, n_{b1} is 1-4, 1-3, 1-2, 2-4, 2-3, 3-4, 1, 2, 3, or 4.

In some variations of formula (18) described in the paragraphs above, D1 is safranin-O. In some variations of formula (18) described in the paragraphs above, D2 is safranin-O. In some variations of formula (18) described in the paragraphs above, D3 is safranin-O. In some variations of formula (18) described in the paragraphs above, D1 and D2 are safranin-O. In some variations of formula (18) described in the paragraphs above, D1 and D3 are safranin-O. In some variations of formula (18) described in the paragraphs above, D2 and D3 are safranin-O.

In some variations of formula (18) described in the paragraph above, the pendant phenyl ring of D1 is unsubstituted. In some variations of formula (18) described in the paragraph above, the pendant phenyl ring of D1 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D1 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D1 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (18) described in the two paragraphs above, the pendant phenyl ring of D2 is unsubstituted. In some variations of formula (18) described in the paragraph above, the pendant phenyl ring of D2 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D2 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D2 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (18) described in the three paragraphs above, the pendant phenyl ring of D3 is unsubstituted. In some variations of formula (18) described in the paragraph above, the pendant phenyl ring of D3 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D3 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D3 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

Some cationic dye dimers fall within formula (19):

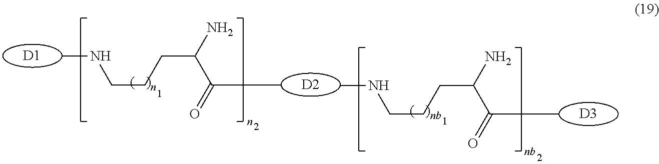

wherein each of D1, D2, and D3 is a cationic dye moiety, n₁ and n_{b1} independently are 0-5, and n₂ and n_{b2} independently are 1-5.

In some variations of formula (19), D1, D2, and D3 independently are selected from the group consisting of safranin-O, toluidine blue, azure A, azure B, azure C, acridine orange, acriflavine, and methylene blue. In some variations of formula (19), D1 and D2 are different cationic dye moieties. In other variations of formula (19), D1 and D2 are the same cationic dye moiety.

In some variations of formula (19) described in the paragraphs above, n₁ is 0-5, 0-4, 0-3, 0-2, 0-1, 1-5, 1-4, 1-3, 1-2, 2-5, 2-4, 2-3, 3-5, 3-4, 4-5, 0, 1, 2, 3, 4, or 5.

In some variations of formula (19) described in the paragraphs above, n_{b1} is 0-5, 0-4, 0-3, 0-2, 0-1, 1-5, 1-4, 1-3, 1-2, 2-5, 2-4, 2-3, 3-5, 3-4, 4-5, 0, 1, 2, 3, 4, or 5.

In some variations of formula (19) described in the paragraphs above, n₂ is 1-5, 1-4, 1-3, 1-2, 2-5, 2-4, 2-3, 3-5, 3-4, 4-5, 1, 2, 3, 4, or 5.

In some variations of formula (19) described in the paragraphs above, n_{b2} is 1-5, 1-4, 1-3, 1-2, 2-5, 2-4, 2-3, 3-5, 3-4, 4-5, 1, 2, 3, 4, or 5.

In some variations of formula (19) described in the paragraphs above, D1 is safranin-O. In some variations of formula (19) described in the paragraphs above, D2 is safranin-O. In some variations of formula (19) described in the paragraphs above, D3 is safranin-O. In some variations of formula (19) described in the paragraphs above, D1 and D2 are safranin-O. In some variations of formula (19) described in the paragraphs above, D1 and D3 are safranin-O. In some variations of formula (19) described in the paragraphs above, D2 and D3 are safranin-O.

In some variations of formula (19) described in the paragraph above, the pendant phenyl ring of D1 is unsubstituted. In some variations of formula (19) described in the paragraph above, the pendant phenyl ring of D1 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D1 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D1 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (19) described in the two paragraphs above, the pendant phenyl ring of D2 is unsubstituted. In some variations of formula (19) described in the paragraph above, the pendant phenyl ring of D2 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D2 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D2 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (19) described in the three paragraphs above, the pendant phenyl ring of D3 is unsubstituted. In some variations of formula (19) described in the paragraph above, the pendant phenyl ring of D3 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D3 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D3 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

Some cationic dye trimers fall within formula (20):

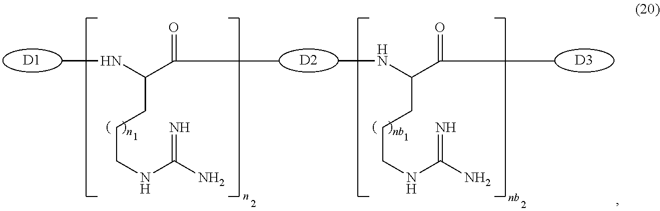

in which each of D1 and D2 is a cationic dye moiety, n₁ and n_{b1} independently are 0-5 and n₂ and nb₂ independently are 1-5.

In some variations of formula (20), each of D1, D2, and D3 independently is selected from the group consisting of safranin-O, toluidine blue, azure A, azure B, azure C, acridine orange, acriflavine, and methylene blue. In some variations of formula (20), D1, D2, and D3 are different cationic dye moieties. In some variations of formula (20), D1, D2, and D3 are the same cationic dye moiety. In other variations of formula (20), D1 and D2 are the same cationic dye moiety and D3 is a different cationic dye moiety. In other variations of formula (20), D1 and D3 are the same cationic dye moiety and D2 is a different cationic dye moiety. In other variations of formula (20), D2 and D3 are the same cationic dye moiety and D1 is a different cationic dye moiety.

In some variations of formula (20) described in the paragraphs above, n₁ is 0-5, 0-4, 0-3, 0-2, 0-1, 1-5, 1-4, 1-3, 1-2, 2-5, 2-4, 2-3, 3-5, 3-4, 4-5, 0, 1, 2, 3, 4, or 5.

In some variations of formula (20) described in the paragraphs above, n₂ is 1-5, 1-4, 1-3, 1-2, 2-5, 2-4, 2-3, 3-5, 3-4, 4-5, 1, 2, 3, 4, or 5.

In some variations of formula (20) described in the paragraphs above, n_{b1} is 0-5, 0-4, 0-3, 0-2, 0-1, 1-5, 1-4, 1-3, 1-2, 2-5, 2-4, 2-3, 3-5, 3-4, 4-5, 0, 1, 2, 3, 4, or 5.

In some variations of formula (20) described in the paragraphs above, nb₂ is 1-5, 1-4, 1-3, 1-2, 2-5, 2-4, 2-3, 3-5, 3-4, 4-5, 1, 2, 3, 4, or 5.

In some variations of formula (20) described in the paragraphs above, D1 is safranin-O.

In some variations of formula (20) described in the paragraphs above, D2 is safranin-O. In some variations of formula (20) described in the paragraphs above, D3 is safranin-O. In some variations of formula (20) described in the paragraphs above, D1 and D2 are safranin-O. In some variations of formula (20) described in the paragraphs above, D1 and D3 are safranin-O. In some variations of formula (20) described in the paragraphs above, D2 and D3 are safranin-O.

In some variations of formula (20) described in the paragraph above, the pendant phenyl ring of D1 is unsubstituted. In some variations of formula (20) described in the paragraph above, the pendant phenyl ring of D1 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D1 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D1 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (20) described in the two paragraphs above, the pendant phenyl ring of D2 is unsubstituted. In some variations of formula (20) described in the paragraph above, the pendant phenyl ring of D2 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D2 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D2 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (20) described in the three paragraphs above, the pendant phenyl ring of D3 is unsubstituted. In some variations of formula (20) described in the paragraph above, the pendant phenyl ring of D3 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D3 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D3 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

Some cationic dye trimers fall within formula (21):

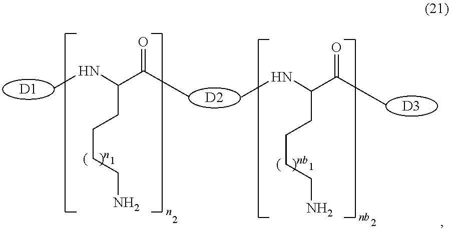

in which each of D1, D2, and D3 is a cationic dye moiety, n₁ and n_{b1} independently are 0-5 and n₂ and nb₂ independently are 1-5.

In some variations of formula (21), D1, D2, and D3 are different cationic dye moieties. In some variations of formula (21), D1, D2, and D3 are the same cationic dye moiety. In other variations of formula (21), D1 and D2 are the same cationic dye moiety and D3 is a different cationic dye moiety. In other variations of formula (21), D1 and D3 are the same cationic dye moiety and D2 is a different cationic dye moiety. In other variations of formula (21), D2 and D3 are the same cationic dye moiety and D1 is a different cationic dye moiety. In some variations of formula (21), each of D1, D2, and D3 independently is selected from the group consisting of safranin-O, toluidine blue, azure A, azure B, azure C, acridine orange, acriflavine, and methylene blue.

In some variations of formula (21) described in the paragraphs above, n₁ is 0-5, 0-4, 0-3, 0-2, 0-1, 1-5, 1-4, 1-3, 1-2, 2-5, 2-4, 2-3, 3-5, 3-4, 4-5, 0, 1, 2, 3, 4, or 5.

In some variations of formula (21) described in the paragraphs above, n₂ is 1-5, 1-4, 1-3, 1-2, 2-5, 2-4, 2-3, 3-5, 3-4, 4-5, 1, 2, 3, 4, or 5.

In some variations of formula (21) described in the paragraphs above, n_{b1} is 0-5, 0-4, 0-3, 0-2, 0-1, 1-5, 1-4, 1-3, 1-2, 2-5, 2-4, 2-3, 3-5, 3-4, 4-5, 0, 1, 2, 3, 4, or 5.

In some variations of formula (21) described in the paragraphs above, n_{b2} is 1-5, 1-4, 1-3, 1-2, 2-5, 2-4, 2-3, 3-5, 3-4, 4-5, 1, 2, 3, 4, or 5.

In some variations of formula (21) described in the paragraphs above, D1 is safranin-O. In some variations of formula (21) described in the paragraphs above, D2 is safranin-O. In some variations of formula (21) described in the paragraphs above, D3 is safranin-O. In some variations of formula (21) described in the paragraphs above, D1 and D2 are safranin-O. In some variations of formula (21) described in the paragraphs above, D1 and D3 are safranin-O. In some variations of formula (21) described in the paragraphs above, D2 and D3 are safranin-O.

In some variations of formula (21) described in the paragraph above, the pendant phenyl ring of D1 is unsubstituted. In some variations of formula (21) described in the paragraph above, the pendant phenyl ring of D1 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D1 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D1 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (21) described in the two paragraphs above, the pendant phenyl ring of D2 is unsubstituted. In some variations of formula (21) described in the paragraph above, the pendant phenyl ring of D2 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D2 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D2 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (21) described in the three paragraphs above, the pendant phenyl ring of D3 is unsubstituted. In some variations of formula (21) described in the paragraph above, the pendant phenyl ring of D3 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D3 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D3 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

Some cationic dye trimers fall within formula (22):

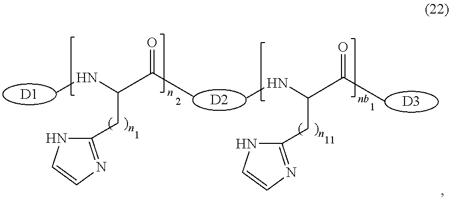

in which each of D1, D2, and D3 is a cationic dye moiety, n₁, n₂, n₁₁, and nb₁ independently are 1-5.

In some variations of formula (22), D1, D2, and D3 are different cationic dye moieties. In some variations of formula (22), D1, D2, and D3 are the same cationic dye moiety. In other variations of formula (22), D1 and D2 are the same cationic dye moiety and D3 is a different cationic dye moiety. In other variations of formula (22), D1 and D3 are the same cationic dye moiety and D2 is a different cationic dye moiety. In other variations of formula (22), D2 and D3 are the same cationic dye moiety and D1 is a different cationic dye moiety. In some variations of formula (22), each of D1, D2, and D3 independently is selected from the group consisting of safranin-O, toluidine blue, azure A, azure B, azure C, acridine orange, acriflavine, and methylene blue.

In some variations of formula (22) described in the paragraphs above, n₁ is 1-5, 1-4, 1-3, 1-2, 2-5, 2-4, 2-3, 3-5, 3-4, 4-5, 1, 2, 3, 4, or 5.

In some variations of formula (22) described in the paragraphs above, n₂ is 1-5, 1-4, 1-3, 1-2, 2-5, 2-4, 2-3, 3-5, 3-4, 4-5, 1, 2, 3, 4, or 5.

In some variations of formula (22) described in the paragraphs above, n₁₁ is 1-5, 1-4, 1-3, 1-2, 2-5, 2-4, 2-3, 3-5, 3-4, 4-5, 1, 2, 3, 4, or 5.

In some variations of formula (22) described in the paragraphs above, nb₁ is 1-5, 1-4, 1-3, 1-2, 2-5, 2-4, 2-3, 3-5, 3-4, 4-5, 1, 2, 3, 4, or 5.

In some variations of formula (22) described in the paragraphs above, D1 is safranin-O. In some variations of formula (22) described in the paragraphs above, D2 is safranin-O. In some variations of formula (22) described in the paragraphs above, D3 is safranin-O. In some variations of formula (22) described in the paragraphs above, D1 and D2 are safranin-O. In some variations of formula (22) described in the paragraphs above, D1 and D3 are safranin-O. In some variations of formula (22) described in the paragraphs above, D2 and D3 are safranin-O.

In some variations of formula (22) described in the paragraph above, the pendant phenyl ring of D1 is unsubstituted. In some variations of formula (22) described in the paragraph above, the pendant phenyl ring of D1 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D1 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D1 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (22) described in the two paragraphs above, the pendant phenyl ring of D2 is unsubstituted. In some variations of formula (22) described in the paragraph above, the pendant phenyl ring of D2 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D2 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D2 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (22) described in the three paragraphs above, the pendant phenyl ring of D3 is unsubstituted. In some variations of formula (22) described in the paragraph above, the pendant phenyl ring of D3 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D3 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D3 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

Some cationic dye trimers fall within formula (23):

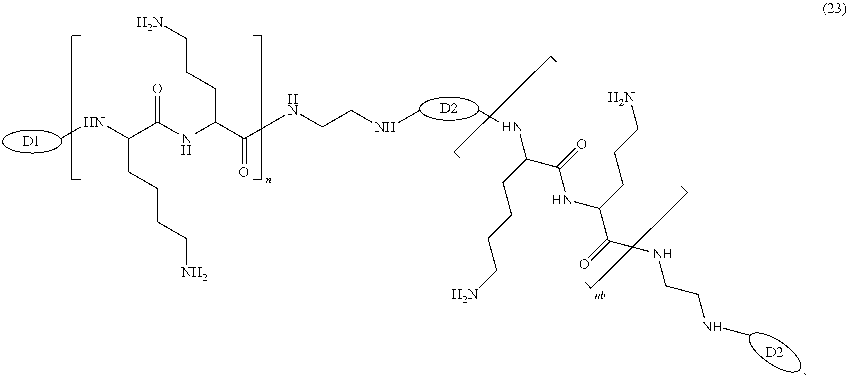

in which each of D1, D2, and D3 is a cationic dye moiety and n and nb independently are 1-6.

In some variations of formula (23), D1, D2, and D3 are different cationic dye moieties. In some variations of formula (23), D1, D2, and D3 are the same cationic dye moiety. In other variations of formula (23), D1 and D2 are the same cationic dye moiety and D3 is a different cationic dye moiety. In other variations of formula (23), D1 and D3 are the same cationic dye moiety and D2 is a different cationic dye moiety. In other variations of formula (23), D2 and D3 are the same cationic dye moiety and D1 is a different cationic dye moiety. In some variations of formula (23), each of D1, D2, and D3 independently is selected from the group consisting of safranin-O, toluidine blue, azure A, azure B, azure C, acridine orange, acriflavine, and methylene blue.

In some variations of formula (23) described above, n is 1-6, 1-5, 1-4, 1-3, 1-2, 2-6, 2-5, 12-4, 2-3, 3-6, 3-5, 3-4, 4-6, 4-5, 5-6, 1, 2, 3, 4, 5, or 6.

In some variations of formula (23) described above, nb is 1-6, 1-5, 1-4, 1-3, 1-2, 2-6, 2-5, 12-4, 2-3, 3-6, 3-5, 3-4, 4-6, 4-5, 5-6, 1, 2, 3, 4, 5, or 6.

In some variations of formula (23) described in the paragraphs above, D1 is safranin-O. In some variations of formula (23) described in the paragraphs above, D2 is safranin-O. In some variations of formula (23) described in the paragraphs above, D3 is safranin-O. In some variations of formula (23) described in the paragraphs above, D1 and D2 are safranin-O. In some variations of formula (23) described in the paragraphs above, D1 and D3 are safranin-O. In some variations of formula (23) described in the paragraphs above, D2 and D3 are safranin-O.

In some variations of formula (23) described in the paragraph above, the pendant phenyl ring of D1 is unsubstituted. In some variations of formula (23) described in the paragraph above, the pendant phenyl ring of D1 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D1 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D1 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (23) described in the two paragraphs above, the pendant phenyl ring of D2 is unsubstituted. In some variations of formula (23) described in the paragraph above, the pendant phenyl ring of D2 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D2 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D2 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (23) described in the three paragraphs above, the pendant phenyl ring of D3 is unsubstituted. In some variations of formula (23) described in the paragraph above, the pendant phenyl ring of D3 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D3 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D3 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

Some cationic dye multimers fall within formula (24):

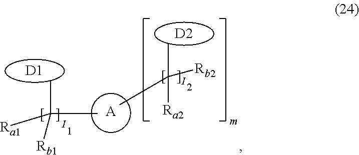

in which m is 2; each of D1, a first D2, and a second D2 is a cationic dye moiety; l₁ and l₂ independently are 1-4; ring A is aryl, heteroaryl, cycloalkyl, or heterocyclyl; for each independent instance of Rₐ₁ and R_{b1}, Rₐ₁ and R_{b1} (1) independently are H or CH₃, or (2) Rₐ₁ and R_{b1} are

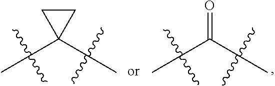

or (3) two of CRₐ₁R_{b1} are

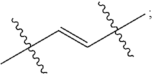 [SMILES: ";" ; */C=C/*]

and, for each independent instance of Rₐ₂ and R_{b2}, Rₐ₂ and R_{b2} (1) independently are H or CH₃, or (2) Rₐ₂ and R_{b2} are

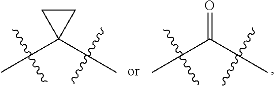

or (3) two of CRₐ₂R_{b2} are

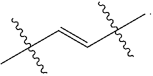 [SMILES: "." ; */C=C/*]

In some variations of formula (24), D1, the first D2, and the second D2 are different cationic dye moieties. In some variations of formula (24), D1, the first D2, and the second D2 are the same cationic dye moiety. In other variations of formula (24), D1 and the first D2 are the same cationic dye moiety and the second D2 is a different cationic dye moiety. In other variations of formula (24), D1 and the second D2 are the same cationic dye moiety and the first D2 is a different cationic dye moiety. In other variations of formula (24), the first D2 and the second D2 are the same cationic dye moiety and D1 is a different cationic dye moiety. In some variations of formula (24), each of D1, the first D2, and the second D2 independently is selected from the group consisting of safranin-O, toluidine blue, azure A, azure B, azure C, acridine orange, acriflavine, and methylene blue.

In some variations of formula (24), l₁ is 1-4, 1-3, 1-2, 2-4, 2-3, 3-4, 1, 2, 3, or 4.

In some variations of formula (24) described in the paragraphs above, l₂ is 1-4, 1-3, 1-2, 2-4, 2-3, 3-4, 1, 2, 3, or 4.

In some variations of formula (24) described in the paragraphs above in which l₁ is 1, Rₐ₁ and R_{b1} are both H. In some variations of formula (24) described in the paragraphs above in which l₁ is 1, Rₐ₁ is H and R_{b1} is CH₃. In some variations of formula (24) described in the paragraphs above in which l₁ is 1, Rₐ₁ and R_{b1} are both CH₃. In some variations of formula (24) described in the paragraphs above in which l₁ is 1, Rₐ₁ and R_{b1} are

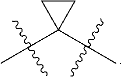 [SMILES: *C1(*)CC1 ; "."]

In some variations of formula (24) described in the paragraphs above in which l₁ is 1, Rₐ₁ and R_{b1} are

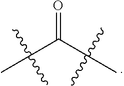 [SMILES: *C(*)=O ; "."]

In variations of formula (24) described in the paragraphs above in which l₁ is 2, the two instances of Rₐ and R_{b} are indicated as Rₐ₁ and R_{b1} and Rₐ₂ and R_{b2}, respectively. In some variations of formula (24) described in the paragraphs above in which l₁ is 2, each of Rₐ₁ and R_{b1} and Rₐ₂ and R_{b2} is H. In some variations of formula (24) described in the paragraphs above in which l₁ is 2, each of Rₐ₁ and R_{b1} and Rₐ₂ and R_{b2} is CH₃. In some variations of formula (24) described in the paragraphs above in which l₁ is 2, each of Rₐ₁ and Rₐ₂ is H and each of R_{b1} and R_{b2} is CH₃. In some variations of formula (24) described in the paragraphs above in which l₁ is 2, each of Rₐ₁, Rₐ₂, and R_{b1} is H and R_{b2} is CH₃.

In some variations of formula (24) described in the paragraphs above in which l₁ is 2, each of Rₐ₁ and R_{b1} is H and Rₐ₂ and R_{b2} are

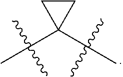 [SMILES: *C1(*)CC1 ; "."]

In some variations of formula (24) described in the paragraphs above in which l₁ is 2, each of Rₐ₁ and R_{b1} is H and Rₐ₂ and R_{b2} are

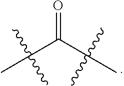 [SMILES: *C(*)=O ; "."]

In some variations of formula (24) described in the paragraphs above in which l₁ is 2, each of Rₐ₁ and R_{b1} is CH₃ and Rₐ₂ and R_{b2} are

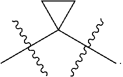 [SMILES: *C1(*)CC1 ; "."]

In some variations of formula (24) described in the paragraphs above in which l₁ is 2, each of Rₐ₁ and R_{b1} is CH₃ and Rₐ₂ and R_{b2} are

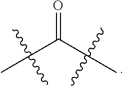 [SMILES: *C(*)=O ; "."]

In some variations of formula (24) described in the paragraphs above in which l₁ is 2, Rₐ₁ is H, R_{b1} is CH₃, and Rₐ₂ and R_{b2} are

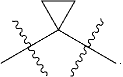 [SMILES: *C1(*)CC1 ; "."]

In some variations of formula (24) described in the paragraphs above in which l₁ is 2, Rₐ₁ is H, R_{b1} is CH₃, and Rₐ₂ and R_{b2} are

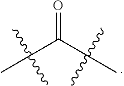 [SMILES: *C(*)=O ; "."]

In some variations of formula (24) described in the paragraphs above in which l₁ is 2, Rₐ₁ and R_{b1} and Rₐ₂ and R_{b2} together are

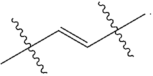 [SMILES: "." ; */C=C/*]

In some variations of formula (24) described in the paragraphs above in which l₁ is 2, Rₐ₁ and R_{b1} are

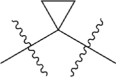 [SMILES: *C1(*)CC1]

Rₐ₂ and R_{b2} are

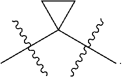 [SMILES: *C1(*)CC1 ; "."]

In some variations of formula (24) described in the paragraphs above in which l₁ is 2, Rₐ₁ and R_{b1} are

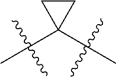 [SMILES: *C1(*)CC1]

Rₐ₂ and R_{b2} are

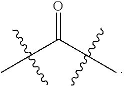 [SMILES: *C(*)=O ; "."]

In some variations of formula (24) described in the paragraphs above in which l₁ is 2, Rₐ₁ and R_{b1} are

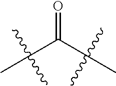 [SMILES: *C(*)=O]

Rₐ₂ and R_{b2} are

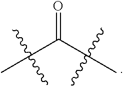 [SMILES: *C(*)=O ; "."]

In variations of formula (24) described in the paragraphs above in which l₁ is 3, the three instances of Rₐ and R_{b} are indicated as Rₐ₁ and R_{b1}; Rₐ₂ and R_{b2}; and Rₐ₃ and R_{b3}, respectively. In some variations of formula (24) described in the paragraphs above in which l₁ is 3, In some variations of formula (24) described in the paragraphs above in which l₁ is 3, each of Rₐ₁, R_{b1}, Rₐ₂, R_{b2}, Rₐ₃, and R_{b3} is H. In some variations of formula (24) described in the paragraphs above in which l₁ is 3, each of Rₐ₁, R_{b1}, Rₐ₂, R_{b2}, Rₐ₃, and R_{b3} is CH₃. In some variations of formula (24) described in the paragraphs above in which l₁ is 3, each of Rₐ₁, R_{b1}, Rₐ₂, and R_{b2}, is H and each of Rₐ₃ and R_{b3} is CH₃. In some variations of formula (24) described in the paragraphs above in which l₁ is 3, each of Rₐ₁, R_{b1}, Rₐ₂, and R_{b2}, is CH₃ and each of Rₐ₃ and R_{b3} is H. In some variations of formula (24) described in the paragraphs above in which l₁ is 3, Rₐ₁ is H and each of Rₐ₂, Rₐ₃, R_{b1}, R_{b2}, and R_{b3} is CH₃. In some variations of formula (24) described in the paragraphs above in which l₁ is 3, each of Rₐ₁ and Rₐ₂ is H and each of Rₐ₃, R_{b1}, R_{b2}, an R_{b3} is CH₃. In some variations of formula (24) described in the paragraphs above in which l₁ is 3, each of Rₐ₁, Rₐ₂, and Rₐ₃ is H and each of R_{b1}, R_{b2}, an R_{b3} is CH₃.

In some variations of formula (24) described in the paragraphs above in which l₁ is 3, each of Rₐ₁, R_{b1}, Rₐ₂, and R_{b2}, is H and Rₐ₃ and R_{b3} are

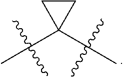 [SMILES: *C1(*)CC1 ; "."]

In some variations of formula (24) described in the paragraphs above in which l₁ is 3, each of Rₐ₁, R_{b1}, Rₐ₂, and R_{b2}, is CH₃ and Rₐ₃ and R_{b3} are

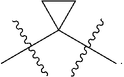 [SMILES: *C1(*)CC1 ; "."]

In some variations of formula (24) described in the paragraphs above in which l₁ is 3, Rₐ₁ is H and each of Rₐ₂, R_{b1}, and R_{b2} is CH₃, and Rₐ₃ and R_{b3} are

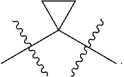 [SMILES: *C1(*)CC1 ; "."]

In some variations of formula (24) described in the paragraphs above in which l₁ is 3, each of Rₐ₁ and Rₐ₂ is H and each of R_{b1} and R_{b2} is CH₃, and Rₐ₃ and R_{b3} are

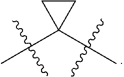 [SMILES: *C1(*)CC1 ; "."]

In some variations of formula (24) described in the paragraphs above in which l₁ is 3, each of Rₐ₁, R_{b1}, Rₐ₂, and R_{b2}, is H and Rₐ₃ and R_{b3} are

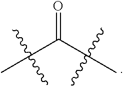 [SMILES: *C(*)=O ; "."]

In some variations of formula (24) described in the paragraphs above in which l₁ is 3, each of Rₐ₁, R_{b1}, Rₐ₂, and R_{b2}, is CH₃ and Rₐ₃ and R_{b3} are

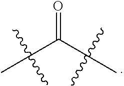 [SMILES: *C(*)=O ; "."]

In some variations of formula (24) described in the paragraphs above in which l₁ is 3, Rₐ₁ is H and each of Rₐ₂, R_{b1}, and R_{b2} is CH₃, and Rₐ₃ and R_{b3} are

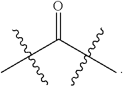 [SMILES: *C(*)=O ; "."]

In some variations of formula (24) described in the paragraphs above in which l₁ is 3, each of Rₐ₁ and Rₐ₂ is H and each of R_{b1} and R_{b2} is CH₃, and Rₐ₃ and R_{b3} are

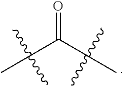 [SMILES: *C(*)=O ; "."]

In some variations of formula (24) described in the paragraphs above in which l₁ is 3, Rₐ₁ and R_{b1} are

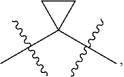 [SMILES: *C1(*)CC1 ; ","]

Rₐ₂ and R_{b2} are

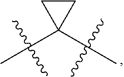 [SMILES: *C1(*)CC1 ; ","]

and each of Rₐ₃ and R_{b3} is H. In some variations of formula (24) described in the paragraphs above in which l₁ is 3, Rₐ₁ and R_{b1} are

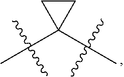 [SMILES: *C1(*)CC1 ; ","]

Rₐ₂ and R_{b2} are

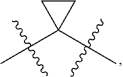 [SMILES: *C1(*)CC1 ; ","]

and each of Rₐ₃ and R_{b3} is CH₃. In some variations of formula (24) described in the paragraphs above in which l₁ is 3, Rₐ₁ and R_{b1} are

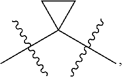 [SMILES: *C1(*)CC1 ; ","]

Rₐ₂ and R_{b2} are

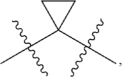 [SMILES: *C1(*)CC1 ; ","]

Rₐ₃ is H, and R_{b3} is CH₃. In some variations of formula (24) described in the paragraphs above in which l₁ is 3, Rₐ₁ and R_{b1} are

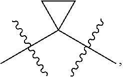 [SMILES: *C1(*)CC1 ; ","]

Rₐ₂ and R_{b2} are

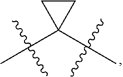 [SMILES: *C1(*)CC1 ; ","]

and each of Rₐ₃ and R_{b3} is H. In some variations of formula (24) described in the paragraphs above in which l₁ is 3, Rₐ₁ and R_{b1} are

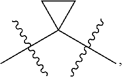 [SMILES: *C1(*)CC1 ; ","]

Rₐ₂ and R_{b2} are

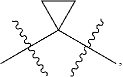 [SMILES: *C1(*)CC1 ; ","]

and each of Rₐ₃ and R_{b3} is CH₃. In some variations of formula (24) described in the paragraphs above in which l₁ is 3, Rₐ₁ and R_{b1} are

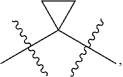 [SMILES: *C1(*)CC1 ; ","]

Rₐ₂ and R_{b2} are

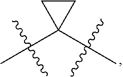 [SMILES: *C1(*)CC1 ; ","]

Rₐ₃ is H, and R_{b3} is CH₃.

In some variations of formula (24) described in the paragraphs above in which l₁ is 3, Rₐ₁ and R_{b1} are

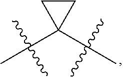 [SMILES: *C1(*)CC1 ; ","]

Rₐ₂ and R_{b2} are

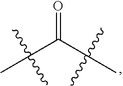 [SMILES: *C(*)=O ; ","]

and each of Rₐ₃ and R_{b3} is H. In some variations of formula (24) described in the paragraphs above in which l₁ is 3, Rₐ₁ and R_{b1} are

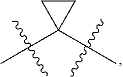 [SMILES: *C1(*)CC1 ; ","]

Rₐ₂ and R_{b2} are

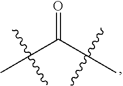 [SMILES: *C(*)=O ; ","]

and each of Rₐ₃ and R_{b3} is CH₃. In some variations of formula (24) described in the paragraphs above in which l₁ is 3, Rₐ₁ and R_{b1} are

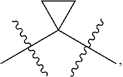 [SMILES: *C1(*)CC1 ; ","]

Rₐ₂ and R_{b2} are

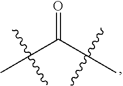 [SMILES: *C(*)=O ; ","]

Rₐ₃ is H, and R_{b3} is CH₃. In some variations of formula (24) described in the paragraphs above in which l₁ is 3, Rₐ₁ and R_{b1} are

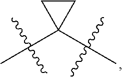 [SMILES: *C1(*)CC1]

Rₐ₂ and R_{b2} are

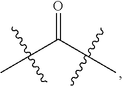 [SMILES: *C(*)=O]

and each of Rₐ₃ and R_{b3} is H. In some variations of formula (24) described in the paragraphs above in which l₁ is 3, Rₐ₁ and R_{b1} are

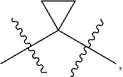 [SMILES: *C1(*)CC1]

Rₐ₂ and R_{b2} are

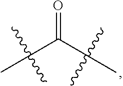 [SMILES: *C(*)=O]

and each of Rₐ₃ and R_{b3} is CH₃. In some variations of formula (24) described in the paragraphs above in which l₁ is 3, Rₐ₁ and R_{b1} are

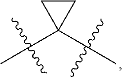 [SMILES: *C1(*)CC1]

Rₐ₂ and R_{b2} are

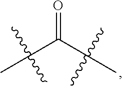 [SMILES: *C(*)=O]

Rₐ₃ is H, and R_{b3} is CH₃.

In some variations of formula (24) described in the paragraphs above in which l₁ is 3, Rₐ₁ and R_{b1} are

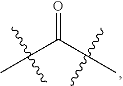 [SMILES: *C(*)=O]

Rₐ₂ and R_{b2} are

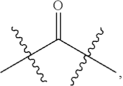 [SMILES: *C(*)=O]

and each of Rₐ₃ and R_{b3} is H. In some variations of formula (24) described in the paragraphs above in which l₁ is 3, Rₐ₁ and R_{b1} are

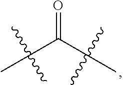 [SMILES: *C(*)=O]

Rₐ₂ and R_{b2} are

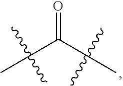 [SMILES: *C(*)=O]

and each of Rₐ₃ and R_{b3} is CH₃. In some variations of formula (24) described in the paragraphs above in which l₁ is 3, Rₐ₁ and R_{b1} are

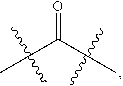 [SMILES: *C(*)=O]

Rₐ₂ and R_{b2} are

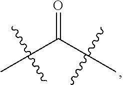 [SMILES: *C(*)=O]

Rₐ₃ is H, and R_{b3} is CH₃. In some variations of formula (24) described in the paragraphs above in which l₁ is 3, Rₐ₁ and R_{b1} are

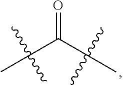 [SMILES: *C(*)=O]

Rₐ₂ and R_{b2} are

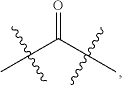 [SMILES: *C(*)=O]

and each of Rₐ₃ and R_{b3} is H. In some variations of formula (24) described in the paragraphs above in which l₁ is 3, Rₐ₁ and R_{b1} are

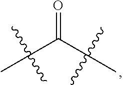 [SMILES: *C(*)=O]

Rₐ₂ and R_{b2} are

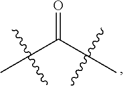 [SMILES: *C(*)=O ; ","]

and each of Rₐ₃ and R_{b3} is CH₃. In some variations of formula (24) described in the paragraphs above in which l₁ is 3, Rₐ₁ and R_{b1} are

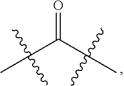 [SMILES: *C(*)=O ; ","]

Rₐ₂ and R_{b2} are

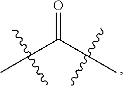 [SMILES: *C(*)=O ; ","]

Rₐ₃ is H, and R_{b3} is CH₃.

In some variations of formula (24) described in the paragraphs above in which l₁ is 3, Rₐ₁ and R_{b1} and Rₐ₂ and R_{b2} together are

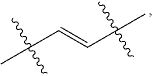 [SMILES: "," ; */C=C/*]

and each of Rₐ₃ and R_{b3} is H. In some variations of formula (24) described in the paragraphs above in which l₁ is 3, Rₐ₁ and R_{b1} and Rₐ₂ and R_{b2} together are

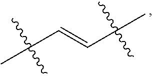 [SMILES: "," ; */C=C/*]

and each of Rₐ₃ and R_{b3} is CH₃. In some variations of formula (24) described in the paragraphs above in which l₁ is 3, Rₐ₁ and R_{b1} and Rₐ₂ and R_{b2} together are

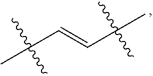 [SMILES: "," ; */C=C/*]

Rₐ₃ is H, and R_{b3} is CH₃. In some variations of formula (24) described in the paragraphs above in which l₁ is 3, Rₐ₁ and R_{b1} and Rₐ₂ and R_{b2} together are

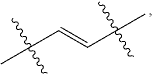 [SMILES: "," ; */C=C/*]

and Rₐ₃ and R_{b3} are

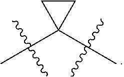 [SMILES: *C1(*)CC1 ; "."]

In some variations of formula (24) described in the paragraphs above in which l₁ is 3, Rₐ₁ and R_{b1} and Rₐ₂ and R_{b2} together are

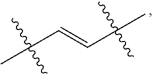 [SMILES: "," ; */C=C/*]

and Rₐ₃ and R_{b3} are

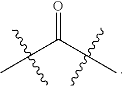 [SMILES: *C(*)=O ; "."]

In variations of formula (24) described in the paragraphs above in which l₁ is 4, the three instances of Rₐ and R_{b} are indicated as Rₐ₁ and R_{b1}; Rₐ₂ and R_{b2}; Rₐ₃ and R_{b3}; and Rₐ₄ and R_{b4}, respectively. In some variations of formula (24) described in the paragraphs above in which l₁ is 4, In some variations of formula (24) described in the paragraphs above in which l₁ is 4, each of Rₐ₁, R_{b1}, Rₐ₂, R_{b2}, Rₐ₃, R_{b3}, Rₐ₄, and R_{b4} is H. In some variations of formula (24) described in the paragraphs above in which l₁ is 4, each of Rₐ₂, R_{b2}, Rₐ₃, R_{b3}, Rₐ₄, and R_{b4} is CH₃. In some variations of formula (24) described in the paragraphs above in which l₁ is 4, each of Rₐ₁, R_{b1}, Rₐ₂, R_{b2}, Rₐ₃, and R_{b3} is H and each of Rₐ₄ and R_{b4} is CH₃. In some variations of formula (24) described in the paragraphs above in which l₁ is 4, each of Rₐ₁, R_{b1}, Rₐ₂, and R_{b2} is H and each of Rₐ₃, R_{b3}, Rₐ₄, and R_{b4} is CH₃. In some variations of formula (24) described in the paragraphs above in which l₁ is 4, each of Rₐ₁, R_{b1}, Rₐ₂, R_{b2}, Rₐ₃, and R_{b3} is CH₃ and each of Rₐ₄ and R_{b4} is H.

In some variations of formula (24) described in the paragraphs above in which l₁ is 4, each of Rₐ₁, Rₐ₂, and Rₐ₃ is H and each of R_{b1}, R_{b2}, and R_{b3} is CH₃. In some variations of formula (24) described in the paragraphs above in which l₁ is 4, Rₐ₁ is H, R_{b1} is CH₃, and each of Rₐ₂, R_{b2}, Rₐ₃, and R_{b3} is CH₃. In some variations of formula (24) described in the paragraphs above in which l₁ is 4, Rₐ₁ is H, R_{b1} is CH₃, and each of Rₐ₂, R_{b2}, Rₐ₃, and R_{b3} is H. In some variations of formula (24) described in the paragraphs above in which l₁ is 4, each of Rₐ₁ and Rₐ₂ is H, each of R_{b1} and R_{b2} is CH₃, and each of Rₐ₃ and R_{b3} is H. In some variations of formula (24) described in the paragraphs above in which l₁ is 4, each of Rₐ₁ and Rₐ₂ is H, each of R_{b1} and R_{b2} is CH₃, and each of Rₐ₃ and R_{b3} is CH₃.

In some variations of formula (24) described in the paragraphs above in which l₁ is 4, Rₐ₁ and R_{b1} are H, Rₐ₂ and R_{b2} are H, Rₐ₃ and R_{b3} are H, and Rₐ₄ and R_{b4} are

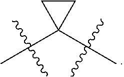 [SMILES: *C1(*)CC1 ; "."]

In some variations of formula (24) described in the paragraphs above in which l₁ is 4, Rₐ₁ and R_{b1} are H, Rₐ₂ and R_{b2} are H, Rₐ₃ and R_{b3} are CH₃, and Rₐ₄ and R_{b4} are

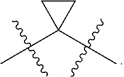 [SMILES: *C1(*)CC1 ; "."]

In some variations of formula (24) described in the paragraphs above in which l₁ is 4, Rₐ₁ and R_{b1} are H, Rₐ₂ and R_{b2} are CH₃, Rₐ₃ and R_{b3} are CH₃, and Rₐ₄ and R_{b4} are

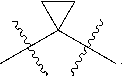 [SMILES: *C1(*)CC1 ; "."]

In some variations of formula (24) described in the paragraphs above in which l₁ is 4, Rₐ₁ and R_{b1} are CH₃, Rₐ₂ and R_{b2} are CH₃, Rₐ₃ and R_{b3} are CH₃, and Rₐ₄ and R_{b4} are

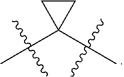 [SMILES: *C1(*)CC1 ; "."]

In some variations of formula (24) described in the paragraphs above in which l₁ is 4, Rₐ₁ and R_{b1} are H, Rₐ₂ and R_{b2} are H, Rₐ₃ and R_{b3} are

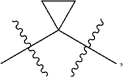 [SMILES: *C1(*)CC1 ; ","]

and Rₐ₄ and R_{b4} are

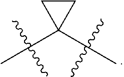 [SMILES: *C1(*)CC1 ; "."]

In some variations of formula (24) described in the paragraphs above in which l₁ is 4, Rₐ₁ and R_{b1} are H, Rₐ₂ and R_{b2} are CH₃, Rₐ₃ and R_{b3} are

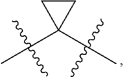 [SMILES: *C1(*)CC1 ; ","]

and Rₐ₄ and R_{b4} are

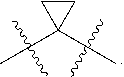 [SMILES: *C1(*)CC1 ; "."]

In some variations of formula (24) described in the paragraphs above in which l₁ is 4, Rₐ₁ and R_{b1} are CH₃, Rₐ₂ and R_{b2} are CH₃, Rₐ₃ and R_{b3} are

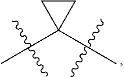 [SMILES: *C1(*)CC1 ; ","]

and Rₐ₄ and R_{b4} are

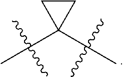 [SMILES: *C1(*)CC1 ; "."]

In some variations of formula (24) described in the paragraphs above in which l₁ is 4, Rₐ₁ is H, R_{b1} is CH₃, Rₐ₂ and R_{b2} are CH₃, Rₐ₃ and R_{b3} are

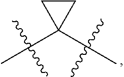 [SMILES: *C1(*)CC1 ; ","]

and Rₐ₄ and R_{b4} are

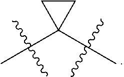 [SMILES: *C1(*)CC1 ; "."]

In some variations of formula (24) described in the paragraphs above in which l₁ is 4, Rₐ₁ is H, R_{b1} is CH₃, Rₐ₂ is H, R_{b2} is CH₃, Rₐ₃ and R_{b3} are

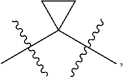 [SMILES: *C1(*)CC1 ; ","]

and Rₐ₄ and R_{b4} are

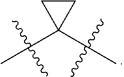 [SMILES: *C1(*)CC1 ; "."]

In some variations of formula (24) described in the paragraphs above in which l₁ is 4, Rₐ₁ and R_{b1} are H, Rₐ₂ and R_{b2} are

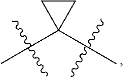 [SMILES: *C1(*)CC1 ; ","]

Rₐ₃ and R_{b3} are

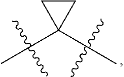 [SMILES: *C1(*)CC1 ; ","]

and Rₐ₄ and R_{b4} are

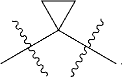 [SMILES: *C1(*)CC1 ; "."]

In some variations of formula (24) described in the paragraphs above in which l₁ is 4, Rₐ₁ and R_{b1} are CH₃, Rₐ₂ and R_{b2} are

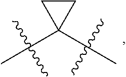 [SMILES: *C1(*)CC1]

Rₐ₃ and R_{b3} are

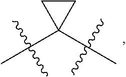 [SMILES: *C1(*)CC1]

and Rₐ₄ and R_{b4} are

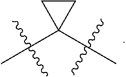 [SMILES: *C1(*)CC1]

In some variations of formula (24) described in the paragraphs above in which l₁ is 4, Rₐ₁ is H, R_{b1} is CH₃, Rₐ₂ and R_{b2} are

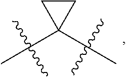 [SMILES: *C1(*)CC1]

Rₐ₃ and R_{b3} are

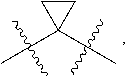 [SMILES: *C1(*)CC1]

and Rₐ₄ and R_{b4} are

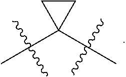 [SMILES: *C1(*)CC1]

In some variations of formula (24) described in the paragraphs above in which l₁ is 4, Rₐ₁ and R_{b1} are

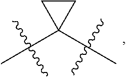 [SMILES: *C1(*)CC1]

Rₐ₂ and R_{b2} are

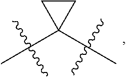 [SMILES: *C1(*)CC1]

Rₐ₃ and R_{b3} are

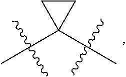 [SMILES: *C1(*)CC1]

and Rₐ₄ and R_{b4} are

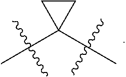 [SMILES: *C1(*)CC1]

In some variations of formula (24) described in the paragraphs above in which l₁ is 4, Rₐ₁ and R_{b1} are H, Rₐ₂ and R_{b2} are H, Rₐ₃ and R_{b3} are H, and Rₐ₄ and R_{b4} are

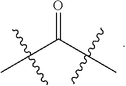 [SMILES: *C(*)=O]

In some variations of formula (24) described in the paragraphs above in which l₁ is 4, Rₐ₁ and R_{b1} are H, Rₐ₂ and R_{b2} are H, Rₐ₃ and R_{b3} are CH₃, and Rₐ₄ and R_{b4} are

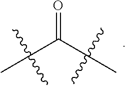 [SMILES: *C(*)=O]

In some variations of formula (24) described in the paragraphs above in which l₁ is 4, Rₐ₁ and R_{b1} are H, Rₐ₂ and R_{b2} are CH₃, Rₐ₃ and R_{b3} are CH₃, and Rₐ₄ and R_{b4} are

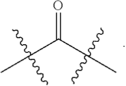 [SMILES: *C(*)=O]

In some variations of formula (24) described in the paragraphs above in which l₁ is 4, Rₐ₁ and R_{b1} are CH₃, Rₐ₂ and R_{b2} are CH₃, Rₐ₃ and R_{b3} are CH₃, and Rₐ₄ and R_{b4} are

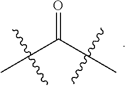 [SMILES: *C(*)=O]

In some variations of formula (24) described in the paragraphs above in which l₁ is 4, Rₐ₁ and R_{b1} are H, Rₐ₂ and R_{b2} are H, Rₐ₃ and R_{b3} are

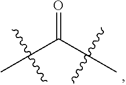 [SMILES: *C(*)=O]

and Rₐ₄ and R_{b4} are v. In some variations of formula (24) described in the paragraphs above in which l₁ is 4, Rₐ₁ and R_{b1} are H, Rₐ₂ and R_{b2} are CH₃, Rₐ₃ and R_{b3} are

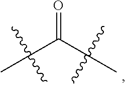 [SMILES: *C(*)=O]

and Rₐ₄ and R_{b4} are

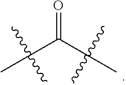 [SMILES: *C(*)=O]

In some variations of formula (24) described in the paragraphs above in which l₁ is 4, Rₐ₁ and R_{b1} are CH₃, Rₐ₂ and R_{b2} are CH₃, Rₐ₃ and R_{b3} are

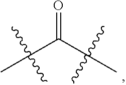 [SMILES: *C(*)=O]

and Rₐ₄ and R_{b4} are

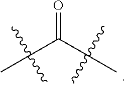 [SMILES: *C(*)=O]

In some variations of formula (24) described in the paragraphs above in which l₁ is 4, Rₐ₁ is H, R_{b1} is CH₃, Rₐ₂ and R_{b2} are CH₃, Rₐ₃ and R_{b3} are

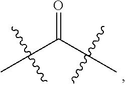 [SMILES: *C(*)=O]

and Rₐ₄ and R_{b4} are

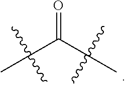 [SMILES: *C(*)=O]

In some variations of formula (24) described in the paragraphs above in which l₁ is 4, Rₐ₁ is H, R_{b1} is CH₃, Rₐ₂ is H, R_{b2} is CH₃, Rₐ₃ and R_{b3} are

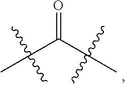 [SMILES: *C(*)=O]

and Rₐ₄ and R_{b4} are

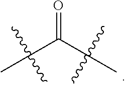 [SMILES: *C(*)=O]

In some variations of formula (24) described in the paragraphs above in which l₁ is 4, Rₐ₁ and R_{b1} are H, Rₐ₂ and R_{b2} are

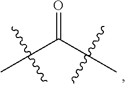 [SMILES: *C(*)=O]

Rₐ₃ and R_{b3} are

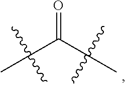 [SMILES: *C(*)=O]

and Rₐ₄ and R_{b4} are

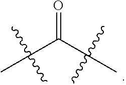 [SMILES: *C(*)=O]

In some variations of formula (24) described in the paragraphs above in which l₁ is 4, Rₐ₁ and R_{b1} are CH₃, Rₐ₂ and R_{b2} are

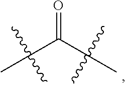 [SMILES: *C(*)=O]

Rₐ₃ and R_{b3} are

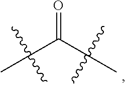 [SMILES: *C(*)=O]

and Rₐ₄ and R_{b4} are

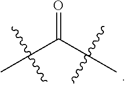 [SMILES: *C(*)=O]

In some variations of formula (24) described in the paragraphs above in which l₁ is 4, Rₐ₁ is H, R_{b1} is CH₃, Rₐ₂ and R_{b2} are

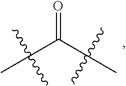 [SMILES: *C(*)=O]

Rₐ₃ and R_{b3} are

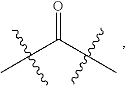 [SMILES: *C(*)=O]

and Rₐ₄ and R_{b4} are

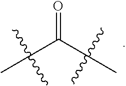 [SMILES: *C(*)=O]

In some variations of formula (24) described in the paragraphs above in which l₁ is 4, Rₐ₁ and R_{b1} are

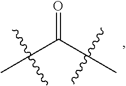 [SMILES: *C(*)=O]

Rₐ₂ and R_{b2} are

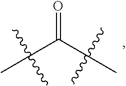 [SMILES: *C(*)=O]

Rₐ₃ and R_{b3} are

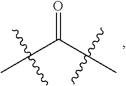 [SMILES: *C(*)=O]

and Rₐ₄ and R_{b4} are

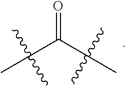 [SMILES: *C(*)=O]

In some variations of formula (24) described in the paragraphs above in which l₁ is 4, Rₐ₁ and R_{b1} are

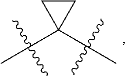 [SMILES: *C1(*)CC1]

Rₐ₂ and R_{b2} are H, Rₐ₃ and R_{b3} are H, and Rₐ₄ and R_{b4} are

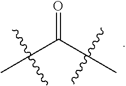 [SMILES: *C(*)=O]

In some variations of formula (24) described in the paragraphs above in which l₁ is 4, Rₐ₁ and R_{b1} are

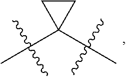 [SMILES: *C1(*)CC1]

Rₐ₂ and R_{b2} are CH₃, Rₐ₃ and R_{b3} are CH₃, and Rₐ₄ and R_{b4} are

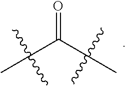 [SMILES: *C(*)=O]

In some variations of formula (24) described in the paragraphs above in which l₁ is 4, Rₐ₁ and R_{b1} are

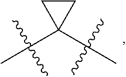 [SMILES: *C1(*)CC1]

Rₐ₂ and R_{b2} are H, Rₐ₃ and R_{b3} are CH₃, and Rₐ₄ and R_{b4} are

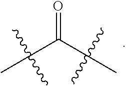 [SMILES: *C(*)=O]

In some variations of formula (24) described in the paragraphs above in which l₁ is 4, Rₐ₁ and R_{b1} are

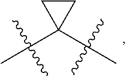 [SMILES: *C1(*)CC1]

Rₐ₂ and R_{b2} are

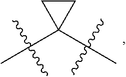 [SMILES: *C1(*)CC1]

Rₐ₃ and R_{b3} are H, and Rₐ₄ and R_{b4} are

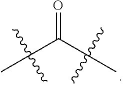 [SMILES: *C(*)=O]

In some variations of formula (24) described in the paragraphs above in which l₁ is 4, Rₐ₁ and R_{b1} are

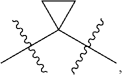 [SMILES: *C1(*)CC1]

Rₐ₂ and R_{b2} are

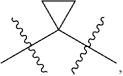 [SMILES: *C1(*)CC1]

Rₐ₃ and R_{b3} are CH₃, and Rₐ₄ and R_{b4} are

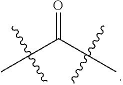 [SMILES: *C(*)=O]

In some variations of formula (24) described in the paragraphs above in which l₁ is 4, Rₐ₁ and R_{b1} are

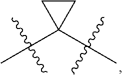 [SMILES: *C1(*)CC1]

Rₐ₂ and R_{b2} are

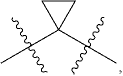 [SMILES: *C1(*)CC1]

Rₐ₃ and R_{b3} are CH₃, and Rₐ₄ and R_{b4} are

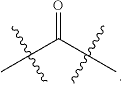 [SMILES: *C(*)=O]

In some variations of formula (24) described in the paragraphs above in which l₁ is 4, Rₐ₁ and R_{b1} are

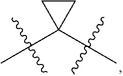 [SMILES: *C1(*)CC1]

Rₐ₂ and R_{b2} are

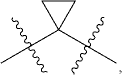 [SMILES: *C1(*)CC1]

Rₐ₃ and R_{b3} are H, and Rₐ₄ and R_{b4} are

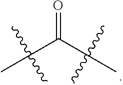 [SMILES: *C(*)=O]

In some variations of formula (24) described in the paragraphs above in which l₁ is 4, Rₐ₁ and R_{b1} are

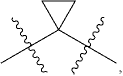 [SMILES: *C1(*)CC1]

Rₐ₂ and R_{b2} are

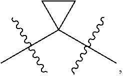 [SMILES: *C1(*)CC1]

Rₐ₃ and R_{b3} are

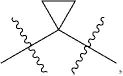 [SMILES: *C1(*)CC1]

and Rₐ₄ and R_{b4} are

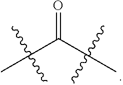 [SMILES: *C(*)=O]

In some variations of formula (24) described in the paragraphs above in which l₁ is 4, Rₐ₁ and R_{b1} are

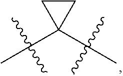 [SMILES: *C1(*)CC1]

Rₐ₂ and R_{b2} are

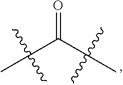 [SMILES: *C(*)=O]

Rₐ₃ and R_{b3} are H, and Rₐ₄ and R_{b4} are

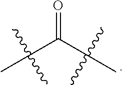 [SMILES: *C(*)=O]

In some variations of formula (24) described in the paragraphs above in which l₁ is 4, Rₐ₁ and R_{b1} are

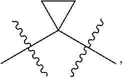 [SMILES: *C1(*)CC1]

Rₐ₂ and R_{b2} are

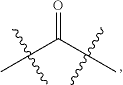 [SMILES: *C(*)=O]

Rₐ₃ and R_{b3} are CH₃, and Rₐ₄ and R_{b4} are

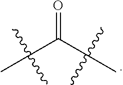 [SMILES: *C(*)=O]

In some variations of formula (24) described in the paragraphs above in which l₁ is 4, Rₐ₁ and R_{b1} are

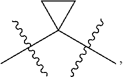 [SMILES: *C1(*)CC1]

Rₐ₂ and R_{b2} are

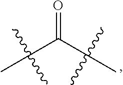 [SMILES: *C(*)=O]

Rₐ₃ and R_{b3} are CH₃, and Rₐ₄ and R_{b4} are

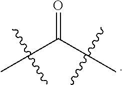 [SMILES: *C(*)=O]

In some variations of formula (24) described in the paragraphs above in which l₁ is 4, Rₐ₁ and R_{b1} are

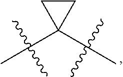 [SMILES: *C1(*)CC1]

Rₐ₂ and R_{b2} are

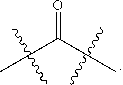 [SMILES: *C(*)=O]

Rₐ₃ and R_{b3} are H, and Rₐ₄ and R_{b4} are

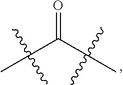 [SMILES: *C(*)=O]

In some variations of formula (24) described in the paragraphs above in which l₁ is 4, Rₐ₁ and R_{b1} are

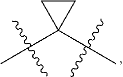 [SMILES: *C1(*)CC1]

Rₐ₂ and R_{b2} are

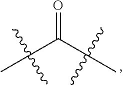 [SMILES: *C(*)=O]

Rₐ₃ and R_{b3} are

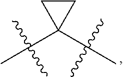 [SMILES: *C1(*)CC1]

and Rₐ₄ and R_{b4} are

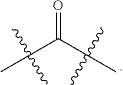 [SMILES: *C(*)=O]

In some variations of formula (24) described in the paragraphs above in which l₁ is 4, Rₐ₁ and R_{b1} are

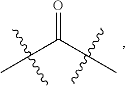 [SMILES: *C(*)=O]

Rₐ₂ and R_{b2} are

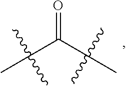 [SMILES: *C(*)=O]

Rₐ₃ and R_{b3} are H, and Rₐ₄ and R_{b4} are

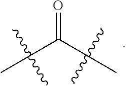 [SMILES: *C(*)=O]

In some variations of formula (24) described in the paragraphs above in which l₁ is 4, Rₐ₁ and R_{b1} are

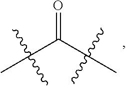 [SMILES: *C(*)=O]

Rₐ₂ and R_{b2} are

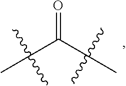 [SMILES: *C(*)=O]

Rₐ₃ and R_{b3} are CH₃, and Rₐ₄ and R_{b4} are

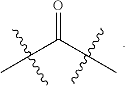 [SMILES: *C(*)=O]

In some variations of formula (24) described in the paragraphs above in which l₁ is 4, Rₐ₁ and R_{b1} are

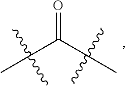 [SMILES: *C(*)=O]

Rₐ₂ and R_{b2} are

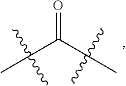 [SMILES: *C(*)=O]

Rₐ₃ and R_{b3} are CH₃, and Rₐ₄ and R_{b4} are

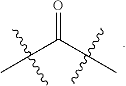 [SMILES: *C(*)=O]

In some variations of formula (24) described in the paragraphs above in which l₁ is 4, Rₐ₁ and R_{b1} are

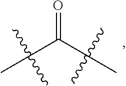 [SMILES: *C(*)=O]

Rₐ₂ and R_{b2} are

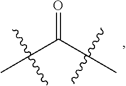 [SMILES: *C(*)=O]

Rₐ₃ and R_{b3} are H, and Rₐ₄ and R_{b4} are

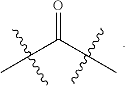 [SMILES: *C(*)=O]

In some variations of formula (24) described in the paragraphs above in which l₁ is 4, Rₐ₁ and R_{b1} are

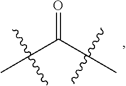 [SMILES: *C(*)=O]

Rₐ₂ and R_{b2} are

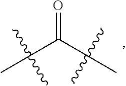 [SMILES: *C(*)=O]

Rₐ₃ and R_{b3} are

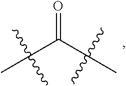 [SMILES: *C(*)=O]

and Rₐ₄ and R_{b4} are

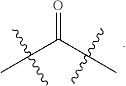 [SMILES: *C(*)=O]

In some variations of formula (24) described in the paragraphs above in which l₁ is 4, Rₐ₁ and R_{b1} are H, Rₐ₂ and R_{b2} are H, and Rₐ₃ and R_{b3} and Rₐ₄ and R_{b4} together are

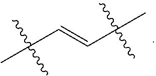 [SMILES: */C=C/*]

In some variations of formula (24) described in the paragraphs above in which l₁ is 4, Rₐ₁ and R_{b1} are H, Rₐ₂ and R_{b2} are CH₃, and Rₐ₃ and R_{b3} and Rₐ₄ and R_{b4} together are

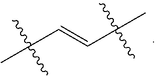 [SMILES: */C=C/*]

In some variations of formula (24) described in the paragraphs above in which l₁ is 4, Rₐ₁ and Rₐ₂ are H, R_{b1} and R_{b2} are CH₃, and Rₐ₃ and R_{b3} and Rₐ₄ and R_{b4} together are

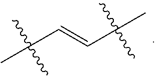 [SMILES: */C=C/*]

In some variations of formula (24) described in the paragraphs above in which l₁ is 4, Rₐ₁ and R_{b1} are H, Rₐ₂ and R_{b2} are

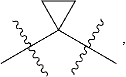 [SMILES: *C1(*)CC1]

and Rₐ₃ and R_{b3} and Rₐ₄ and R_{b4} together are

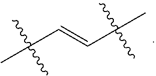 [SMILES: */C=C/*]

In some variations of formula (24) described in the paragraphs above in which l₁ is 4, Rₐ₁ and R_{b1} are

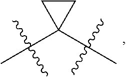 [SMILES: *C1(*)CC1]

Rₐ₂ and R_{b2} are CH₃, and Rₐ₃ and R_{b3} and Rₐ₄ and R_{b4} together are

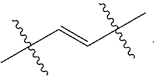 [SMILES: */C=C/*]

In some variations of formula (24) described in the paragraphs above in which l₁ is 4, Rₐ₁ is H, R_{b1} is CH₃, Rₐ₂ and R_{b2} are

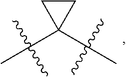 [SMILES: *C1(*)CC1]

and Rₐ₃ and R_{b3} and Rₐ₄ and R_{b4} together are

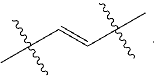 [SMILES: */C=C/*]

In some variations of formula (24) described in the paragraphs above in which l₁ is 4, Rₐ₁ and R_{b1} are H, Rₐ₂ and R_{b2} are

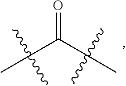 [SMILES: *C(*)=O]

and Rₐ₃ and R_{b3} and Rₐ₄ and R_{b4} together are

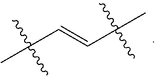 [SMILES: */C=C/*]

In some variations of formula (24) described in the paragraphs above in which l₁ is 4, Rₐ₁ and R_{b1} are

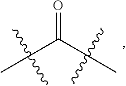 [SMILES: *C(*)=O]

Rₐ₂ and R_{b2} are CH₃, and Rₐ₃ and R_{b3} and Rₐ₄ and R_{b4} together are

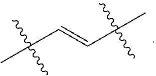 [SMILES: */C=C/*]

In some variations of formula (24) described in the paragraphs above in which l₁ is 4, Rₐ₁ is H, R_{b1} is CH₃, Rₐ₂ and R_{b2} are

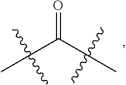 [SMILES: *C(*)=O]

and Rₐ₃ and R_{b3} and Rₐ₄ and R_{b4} together are

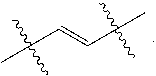 [SMILES: */C=C/*]

In some variations of formula (24) described in the paragraphs above in which l₁ is 4, Rₐ₁ and R_{b1} are

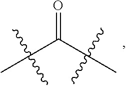 [SMILES: *C(*)=O]

Rₐ₂ and R_{b2} are

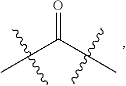 [SMILES: *C(*)=O]

and Rₐ₃ and R_{b3} and Rₐ₄ and R_{b4} together are

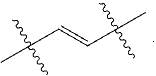 [SMILES: */C=C/*]

In some variations of formula (24) described in the paragraphs above in which l₁ is 4, Rₐ₁ and R_{b1} are

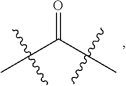 [SMILES: *C(*)=O]

Rₐ₂ and R_{b2} are

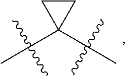 [SMILES: *C1(*)CC1]

and Rₐ₃ and R_{b3} and Rₐ₄ and R_{b4} together are

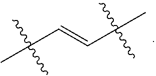 [SMILES: */C=C/*]

In some variations of formula (24) described in the paragraphs above in which l₁ is 4, Rₐ₁ and R_{b1} are

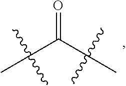 [SMILES: *C(*)=O]

Rₐ₂ and R_{b2} are

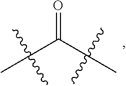 [SMILES: *C(*)=O]

and Rₐ₃ and R_{b3} and Rₐ₄ and R_{b4} together are

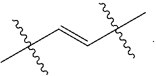 [SMILES: */C=C/*]

In some variations of formula (24) described in the paragraphs above in which l₁ is 4, Rₐ₁ and R_{b1} are

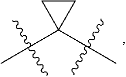 [SMILES: *C1(*)CC1]

Rₐ₂ and R_{b2} are

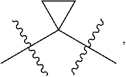 [SMILES: *C1(*)CC1]

and Rₐ₃ and R_{b3} and Rₐ₄ and R_{b4} together are

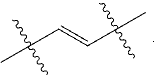 [SMILES: */C=C/*]

In some variations of formula (24) described in the paragraphs above in which l₁ is 4, Rₐ₁ and R_{b1} and Rₐ₂ and R_{b2} together are

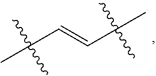 [SMILES: */C=C/*]

and Rₐ₃ and R_{b3} and Rₐ₄ and R_{b4} together are

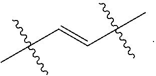 [SMILES: */C=C/*]

In some variations of formula (24) described in the paragraphs above in which l₂ is 1, Rₐ₁ and R_{b1} are both H. In some variations of formula (24) described in the paragraphs above in which l₂ is 1, Rₐ₁ is H and R_{b1} is CH₃. In some variations of formula (24) described in the paragraphs above in which l₂ is 1, Rₐ₁ and R_{b1} are both CH₃. In some variations of formula (24) described in the paragraphs above in which l₂ is 1, Rₐ₁ and R_{b1} are

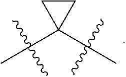 [SMILES: *C1(*)CC1]

In some variations of formula (24) described in the paragraphs above in which l₂ is 1, Rₐ₁ and R_{b1} are

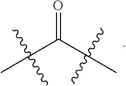 [SMILES: *C(*)=O]

In variations of formula (24) described in the paragraphs above in which l₂ is 2, the two instances of Rₐ and R_{b} are indicated as Rₐ₁ and R_{b1} and Rₐ₂ and R_{b2}, respectively. In some variations of formula (24) described in the paragraphs above in which l₂ is 2, each of Rₐ₁ and R_{b1} and Rₐ₂ and R_{b2} is H. In some variations of formula (24) described in the paragraphs above in which l₂ is 2, each of Rₐ₁ and R_{b1} and Rₐ₂ and R_{b2} is CH₃. In some variations of formula (24) described in the paragraphs above in which l₂ is 2, each of Rₐ₁ and Rₐ₂ is H and each of R_{b1} and R_{b2} is CH₃. In some variations of formula (24) described in the paragraphs above in which l₂ is 2, each of Rₐ₁, Rₐ₂, and R_{b1} is H and R_{b2} is CH₃.

In some variations of formula (24) described in the paragraphs above in which l₂ is 2, each of Rₐ₁ and R_{b1} is H and Rₐ₂ and R_{b2} are

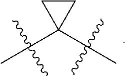 [SMILES: *C1(*)CC1]

In some variations of formula (24) described in the paragraphs above in which l₂ is 2, each of Rₐ₁ and R_{b1} is H and Rₐ₂ and R_{b2} are

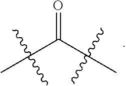 [SMILES: *C(*)=O]

In some variations of formula (24) described in the paragraphs above in which l₂ is 2, each of Rₐ₁ and R_{b1} is CH₃ and Rₐ₂ and R_{b2} are

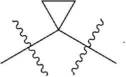 [SMILES: *C1(*)CC1]

In some variations of formula (24) described in the paragraphs above in which l₂ is 2, each of Rₐ₁ and R_{b1} is CH₃ and Rₐ₂ and R_{b2} are

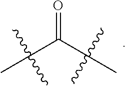 [SMILES: *C(*)=O]

In some variations of formula (24) described in the paragraphs above in which l₂ is 2, Rₐ₁ is H, R_{b1} is CH₃, and Rₐ₂ and R_{b2} are

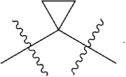 [SMILES: *C1(*)CC1]

In some variations of formula (24) described in the paragraphs above in which l₂ is 2, Rₐ₁ is H, R_{b1} is CH₃, and Rₐ₂ and R_{b2} are

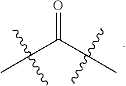 [SMILES: *C(*)=O]

In some variations of formula (24) described in the paragraphs above in which l₂ is 2, Rₐ₁ and R_{b1} and Rₐ₂ and R_{b2} together are

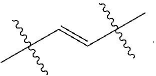 [SMILES: */C=C/*]

In some variations of formula (24) described in the paragraphs above in which l₂ is 2, Rₐ₁ and R_{b1} are

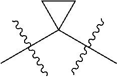 [SMILES: *C1(*)CC1]

Rₐ₂ and R_{b2} are

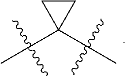 [SMILES: *C1(*)CC1]

In some variations of formula (24) described in the paragraphs above in which l₂ is 2, Rₐ₁ and R_{b1} are

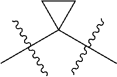 [SMILES: *C1(*)CC1]

Rₐ₂ and R_{b2} are

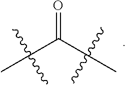 [SMILES: *C(*)=O]

In some variations of formula (24) described in the paragraphs above in which l₂ is 2, Rₐ₁ and R_{b1} are

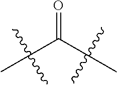 [SMILES: *C(*)=O]

Rₐ₂ and R_{b2} are

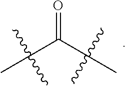 [SMILES: *C(*)=O]

In variations of formula (24) described in the paragraphs above in which l₂ is 3, the three instances of Rₐ and R_{b} are indicated as Rₐ₁ and R_{b1}; Rₐ₂ and R_{b2}; and Rₐ₃ and R_{b3}, respectively. In some variations of formula (24) described in the paragraphs above in which l₂ is 3, In some variations of formula (24) described in the paragraphs above in which l₂ is 3, each of Rₐ₁, R_{b1}, Rₐ₂, R_{b2}, Rₐ₃, and R_{b3} is H. In some variations of formula (24) described in the paragraphs above in which l₂ is 3, each of Rₐ₁, R_{b1}, Rₐ₂, R_{b2}, Rₐ₃, and R_{b3} is CH₃. In some variations of formula (24) described in the paragraphs above in which l₂ is 3, each of Rₐ₁, R_{b1}, Rₐ₂, and R_{b2}, is H and each of Rₐ₃ and R_{b3} is CH₃. In some variations of formula (24) described in the paragraphs above in which l₂ is 3, each of Rₐ₁, R_{b1}, Rₐ₂, and R_{b2}, is CH₃ and each of Rₐ₃ and R_{b3} is H. In some variations of formula (24) described in the paragraphs above in which l₂ is 3, Rₐ₁ is H and each of Rₐ₂, Rₐ₃, R_{b1}, R_{b2}, and R_{b3} is CH₃. In some variations of formula (24) described in the paragraphs above in which l₂ is 3, each of Rₐ₁ and Rₐ₂ is H and each of Rₐ₃, R_{b1}, R_{b2}, an R_{b3} is CH₃. In some variations of formula (24) described in the paragraphs above in which l₂ is 3, each of Rₐ₁, Rₐ₂, and Rₐ₃ is H and each of R_{b1}, R_{b2}, an R_{b3} is CH₃.

In some variations of formula (24) described in the paragraphs above in which l₂ is 3, each of Rₐ₁, R_{b1}, Rₐ₂, and R_{b2}, is H and Rₐ₃ and R_{b3} are

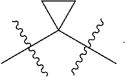 [SMILES: *C1(*)CC1]

In some variations of formula (24) described in the paragraphs above in which l₂ is 3, each of Rₐ₁, R_{b1}, Rₐ₂, and R_{b2}, is CH₃ and Rₐ₃ and R_{b3} are

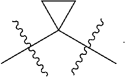 [SMILES: *C1(*)CC1]

In some variations of formula (24) described in the paragraphs above in which l₂ is 3, Rₐ₁ is H and each of Rₐ₂, R_{b1}, and R_{b2} is CH₃, and Rₐ₃ and R_{b3} are

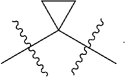 [SMILES: *C1(*)CC1]

In some variations of formula (24) described in the paragraphs above in which l₂ is 3, each of Rₐ₁ and Rₐ₂ is H and each of R_{b1} and R_{b2} is CH₃, and Rₐ₃ and R_{b3} are

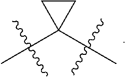 [SMILES: *C1(*)CC1]

In some variations of formula (24) described in the paragraphs above in which l₂ is 3, each of Rₐ₁, R_{b1}, Rₐ₂, and R_{b2}, is H and Rₐ₃ and R_{b3} are

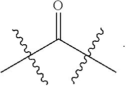 [SMILES: *C(*)=O]

In some variations of formula (24) described in the paragraphs above in which l₂ is 3, each of Rₐ₁, R_{b1}, Rₐ₂, and R_{b2}, is CH₃ and Rₐ₃ and R_{b3} are

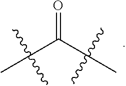 [SMILES: *C(*)=O]

In some variations of formula (24) described in the paragraphs above in which l₂ is 3, Rₐ₁ is H and each of Rₐ₂, R_{b1}, and R_{b2} is CH₃, and Rₐ₃ and R_{b3} are

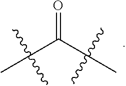 [SMILES: *C(*)=O]

In some variations of formula (24) described in the paragraphs above in which l₂ is 3, each of Rₐ₁ and Rₐ₂ is H and each of R_{b1} and R_{b2} is CH₃, and Rₐ₃ and R_{b3} are

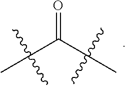 [SMILES: *C(*)=O]

In some variations of formula (24) described in the paragraphs above in which l₂ is 3, Rₐ₁ and R_{b1} are

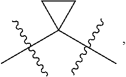 [SMILES: *C1(*)CC1]

Rₐ₂ and R_{b2} are

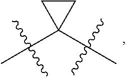 [SMILES: *C1(*)CC1]

and each of Rₐ₃ and R_{b3} is H. In some variations of formula (24) described in the paragraphs above in which l₂ is 3, Rₐ₁ and R_{b1} are

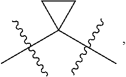 [SMILES: *C1(*)CC1]

Rₐ₂ and R_{b2} are

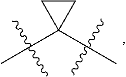 [SMILES: *C1(*)CC1]

and each of Rₐ₃ and R_{b3} is CH₃. In some variations of formula (24) described in the paragraphs above in which l₂ is 3, Rₐ₁ and R_{b1} are

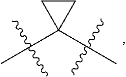 [SMILES: *C1(*)CC1]

Rₐ₂ and R_{b2} are

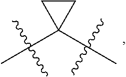 [SMILES: *C1(*)CC1]

Rₐ₃ is H, and R_{b3} is CH₃. In some variations of formula (24) described in the paragraphs above in which l₂ is 3, Rₐ₁ and R_{b1} are

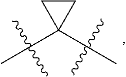 [SMILES: *C1(*)CC1]

Rₐ₂ and R_{b2} are

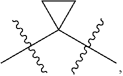 [SMILES: *C1(*)CC1]

and each of Rₐ₃ and R_{b3} is H. In some variations of formula (24) described in the paragraphs above in which l₂ is 3, Rₐ₁ and R_{b1} are

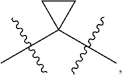 [SMILES: *C1(*)CC1]

Rₐ₂ and R_{b2} are

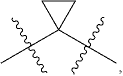 [SMILES: *C1(*)CC1]

and each of Rₐ₃ and R_{b3} is CH₃. In some variations of formula (24) described in the paragraphs above in which l₂ is 3, Rₐ₁ and R_{b1} are

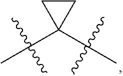 [SMILES: *C1(*)CC1]

Rₐ₂ and R_{b2} are

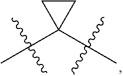 [SMILES: *C1(*)CC1]

Rₐ₃ H, and R_{b3} is CH₃.

In some variations of formula (24) described in the paragraphs above in which l₂ is 3, Rₐ₁ and R_{b1} are

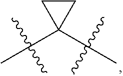 [SMILES: *C1(*)CC1]

Rₐ₂ and R_{b2} are

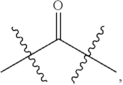 [SMILES: *C(*)=O]

and each of Rₐ₃ and R_{b3} is H. In some variations of formula (24) described in the paragraphs above in which l₂ is 3, Rₐ₁ and R_{b1} are

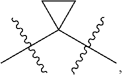 [SMILES: *C1(*)CC1]

Rₐ₂ and R_{b2} are

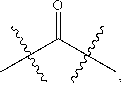 [SMILES: *C(*)=O]

and each of Rₐ₃ and R_{b3} is CH₃. In some variations of formula (24) described in the paragraphs above in which l₂ is 3, Rₐ₁ and R_{b1} are

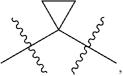 [SMILES: *C1(*)CC1]

Rₐ₂ and R_{b2} are

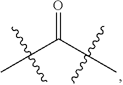 [SMILES: *C(*)=O]

Rₐ₃ is H, and R_{b3} is CH₃. In some variations of formula (24) described in the paragraphs above in which l₂ is 3, Rₐ₁ and R_{b1} are

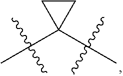 [SMILES: *C1(*)CC1]

Rₐ₂ and R_{b2} are

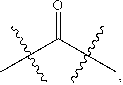 [SMILES: *C(*)=O]

and each of Rₐ₃ and R_{b3} is H. In some variations of formula (24) described in the paragraphs above in which l₂ is 3, Rₐ₁ and R_{b1} are

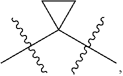 [SMILES: *C1(*)CC1]

Rₐ₂ and R_{b2} are

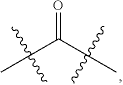 [SMILES: *C(*)=O]

and each of Rₐ₃ and R_{b3} is CH₃. In some variations of formula (24) described in the paragraphs above in which l₂ is 3, Rₐ₁ and R_{b1} are

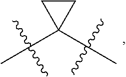 [SMILES: *C1(*)CC1]

Rₐ₂ and R_{b2} are

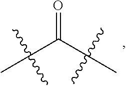 [SMILES: *C(*)=O]

Rₐ₃ is H, and R_{b3} is CH₃.

In some variations of formula (24) described in the paragraphs above in which l₂ is 3, Rₐ₁ and R_{b1} are

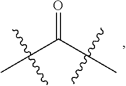 [SMILES: *C(*)=O]

Rₐ₂ and R_{b2} are

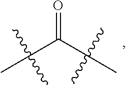 [SMILES: *C(*)=O]

and each of Rₐ₃ and R_{b3} is H. In some variations of formula (24) described in the paragraphs above in which l₂ is 3, Rₐ₁ and R_{b1} are

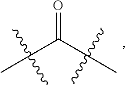 [SMILES: *C(*)=O]

Rₐ₂ and R_{b2} are

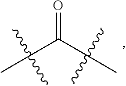 [SMILES: *C(*)=O]

and each of Rₐ₃ and R_{b3} is CH₃. In some variations of formula (24) described in the paragraphs above in which l₂ is 3, Rₐ₁ and R_{b1} are

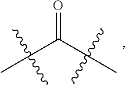 [SMILES: *C(*)=O]

Rₐ₂ and R_{b2} are

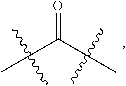 [SMILES: *C(*)=O]

Rₐ₃ is H, and R_{b3} is CH₃. In some variations of formula (24) described in the paragraphs above in which l₂ is 3, Rₐ₁ and R_{b1} are

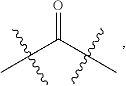 [SMILES: *C(*)=O]

Rₐ₂ and R_{b2} are

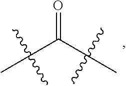 [SMILES: *C(*)=O]

and each of Rₐ₃ and R_{b3} is H. In some variations of formula (24) described in the paragraphs above in which l₂ is 3, Rₐ₁ and R_{b1} are

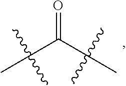 [SMILES: *C(*)=O]

Rₐ₂ and R_{b2} are

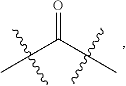 [SMILES: *C(*)=O]

and each of Rₐ₃ and R_{b3} is CH₃. In some variations of formula (24) described in the paragraphs above in which l₂ is 3, Rₐ₁ and R_{b1} are

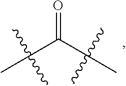 [SMILES: *C(*)=O]

Rₐ₂ and R_{b2} are

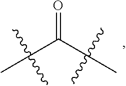 [SMILES: *C(*)=O]

Rₐ₃ is H, and R_{b3} is CH₃.

In some variations of formula (24) described in the paragraphs above in which l₂ is 3, Rₐ₁ and R_{b1} and Rₐ₂ and R_{b2} together are

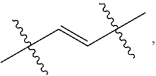 [SMILES: */C=C/*]

and each of Rₐ₃ and R_{b3} is H. In some variations of formula (24) described in the paragraphs above in which l₂ is 3, Rₐ₁ and R_{b1} and Rₐ₂ and R_{b2} together are

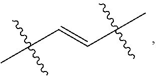 [SMILES: */C=C/*]

and each of Rₐ₃ and R_{b3} is CH₃. In some variations of formula (24) described in the paragraphs above in which l₂ is 3, Rₐ₁ and R_{b1} and Rₐ₂ and R_{b2} together are

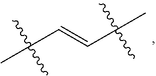 [SMILES: */C=C/*]

Rₐ₃ is H, and R_{b3} is CH₃. In some variations of formula (24) described in the paragraphs above in which l₂ is 3, Rₐ₁ and R_{b1} and Rₐ₂ and R_{b2} together are

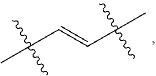 [SMILES: */C=C/*]

and Rₐ₃ and R_{b3} are

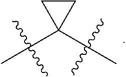 [SMILES: *C1(*)CC1]

In some variations of formula (24) described in the paragraphs above in which l₂ is 3, Rₐ₁ and R_{b1} and Rₐ₂ and R_{b2} together are

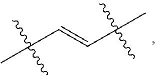 [SMILES: */C=C/*]

and Rₐ₃ and R_{b3} are

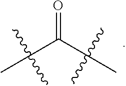 [SMILES: *C(*)=O]

In variations of formula (24) described in the paragraphs above in which l₂ is 4, the three instances of Rₐ and R_{b} are indicated as Rₐ₁ and R_{b1}; Rₐ₂ and R_{b2}; Rₐ₃ and R_{b3}; and Rₐ₄ and R_{b4}, respectively. In some variations of formula (24) described in the paragraphs above in which l₂ is 4, In some variations of formula (24) described in the paragraphs above in which l₂ is 4, each of Rₐ₁, R_{b1}, Rₐ₂, R_{b2}, Rₐ₃, R_{b3}, Rₐ₄, and R_{b4} is H. In some variations of formula (24) described in the paragraphs above in which l₂ is 4, each of Rₐ₁, R_{b1}, Rₐ₂, R_{b2}, Rₐ₃, R_{b3}, Rₐ₄, and R_{b4} is CH₃. In some variations of formula (24) described in the paragraphs above in which l₂ is 4, each of Rₐ₁, R_{b1}, Rₐ₂, R_{b2}, Rₐ₃, and R_{b3} is H and each of Rₐ₄ and R_{b4} is CH₃. In some variations of formula (24) described in the paragraphs above in which l₂ is 4, each of Rₐ₁, R_{b1}, Rₐ₂, and R_{b2} is H and each of Rₐ₃, R_{b3}, Rₐ₄, and R_{b4} is CH₃. In some variations of formula (24) described in the paragraphs above in which l₂ is 4, each of Rₐ₁, R_{b1}, Rₐ₂, R_{b2}, Rₐ₃, and R_{b3} is CH₃ and each of Rₐ₄ and R_{b4} is H.

In some variations of formula (24) described in the paragraphs above in which l₂ is 4, each of Rₐ₁, Rₐ₂, and Rₐ₃ is H and each of R_{b1}, R_{b2}, and R_{b3} is CH₃. In some variations of formula (24) described in the paragraphs above in which l₂ is 4, Rₐ₁ is H, R_{b1} is CH₃, and each of Rₐ₂, R_{b2}, Rₐ₃, and R_{b3} is CH₃. In some variations of formula (24) described in the paragraphs above in which l₂ is 4, Rₐ₁ is H, R_{b1} is CH₃, and each of Rₐ₂, R_{b2}, Rₐ₃, and R_{b3} is H. In some variations of formula (24) described in the paragraphs above in which l₂ is 4, each of Rₐ₁ and Rₐ₂ is H, each of R_{b1} and R_{b2} is CH₃, and each of Rₐ₃ and R_{b3} is H. In some variations of formula (24) described in the paragraphs above in which l₂ is 4, each of Rₐ₁ and Rₐ₂ is H, each of R_{b1} and R_{b2} is CH₃, and each of Rₐ₃ and R_{b3} is CH₃.

In some variations of formula (24) described in the paragraphs above in which l₂ is 4, Rₐ₁ and R_{b1} are H, Rₐ₂ and R_{b2} are H, Rₐ₃ and R_{b3} are H, and Rₐ₄ and R_{b4} are

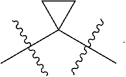 [SMILES: *C1(*)CC1]

In some variations of formula (24) described in the paragraphs above in which l₂ is 4, Rₐ₁ and R_{b1} are H, Rₐ₂ and R_{b2} are H, Rₐ₃ and R_{b3} are CH₃, and Rₐ₄ and R_{b4} are

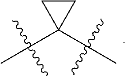 [SMILES: *C1(*)CC1]

In some variations of formula (24) described in the paragraphs above in which l₂ is 4, Rₐ₁ and R_{b1} are H, Rₐ₂ and R_{b2} are CH₃, Rₐ₃ and R_{b3} are CH₃, and Rₐ₄ and R_{b4} are

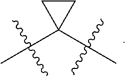 [SMILES: *C1(*)CC1]

In some variations of formula (24) described in the paragraphs above in which l₂ is 4, Rₐ₁ and R_{b1} are CH₃, Rₐ₂ and R_{b2} are CH₃, Rₐ₃ and R_{b3} are CH₃, and Rₐ₄ and R_{b4} are

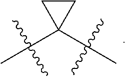 [SMILES: *C1(*)CC1]

In some variations of formula (24) described in the paragraphs above in which l₂ is 4, Rₐ₁ and R_{b1} are H, Rₐ₂ and R_{b2} are H, Rₐ₃ and R_{b3} are

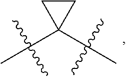 [SMILES: *C1(*)CC1]

and Rₐ₄ and R_{b4} are

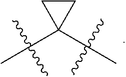 [SMILES: *C1(*)CC1]

In some variations of formula (24) described in the paragraphs above in which l₂ is 4, Rₐ₁ and R_{b1} are H, Rₐ₂ and R_{b2} are CH₃, Rₐ₃ and R_{b3} are

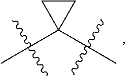 [SMILES: *C1(*)CC1]

and Rₐ₄ and R_{b4} are

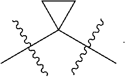 [SMILES: *C1(*)CC1]

In some variations of formula (24) described in the paragraphs above in which l₂ is 4, Rₐ₁ and R_{b1} are CH₃, Rₐ₂ and R_{b2} are CH₃, Rₐ₃ and R_{b3} are

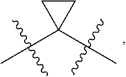 [SMILES: *C1(*)CC1]

and Rₐ₄ and R_{b4} are

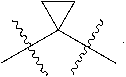 [SMILES: *C1(*)CC1]

In some variations of formula (24) described in the paragraphs above in which l₂ is 4, Rₐ₁ is H, R_{b1} is CH₃, Rₐ₂ and R_{b2} are CH₃, Rₐ₃ and R_{b3} are

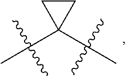 [SMILES: *C1(*)CC1]

and Rₐ₄ and R_{b4} are

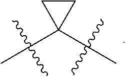 [SMILES: *C1(*)CC1]

In some variations of formula (24) described in the paragraphs above in which l₂ is 4, Rₐ₁ is H, R_{b1} is CH₃, Rₐ₂ is H, R_{b2} is CH₃, Rₐ₃ and R_{b3} are

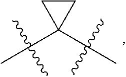 [SMILES: *C1(*)CC1]

and Rₐ₄ and R_{b4} are

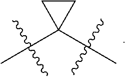 [SMILES: *C1(*)CC1]

In some variations of formula (24) described in the paragraphs above in which l₂ is 4, Rₐ₁ and R_{b1} are H, Rₐ₂ and R_{b2} are

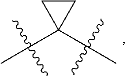 [SMILES: *C1(*)CC1]

Rₐ₃ and R_{b3} are

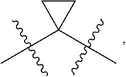 [SMILES: *C1(*)CC1]

and Rₐ₄ and R_{b4} are

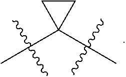 [SMILES: *C1(*)CC1]

In some variations of formula (24) described in the paragraphs above in which l₂ is 4, Rₐ₁ and R_{b1} are CH₃, Rₐ₂ and R_{b2} are

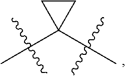 [SMILES: *C1(*)CC1]

Rₐ₃ and R_{b3} are

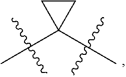 [SMILES: *C1(*)CC1]

and Rₐ₄ and R_{b4} are

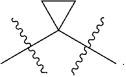 [SMILES: *C1(*)CC1]

In some variations of formula (24) described in the paragraphs above in which l₂ is 4, Rₐ₁ is H, R_{b1} is CH₃, Rₐ₂ and R_{b2} are

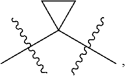 [SMILES: *C1(*)CC1]

Rₐ₃ and R_{b3} are

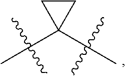 [SMILES: *C1(*)CC1]

and Rₐ₄ and R_{b4} are

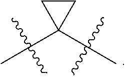 [SMILES: *C1(*)CC1]

In some variations of formula (24) described in the paragraphs above in which l₂ is 4, Rₐ₁ and R_{b1} are

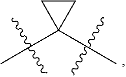 [SMILES: *C1(*)CC1]

Rₐ₂ and R_{b2} are

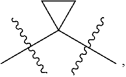 [SMILES: *C1(*)CC1]

Rₐ₃ and R_{b3} are

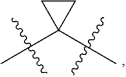 [SMILES: *C1(*)CC1]

and Rₐ₄ and R_{b4} are

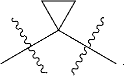 [SMILES: *C1(*)CC1]

In some variations of formula (24) described in the paragraphs above in which l₂ is 4, Rₐ₁ and R_{b1} are H, Rₐ₂ and R_{b2} are H, Rₐ₃ and R_{b3} are H, and Rₐ₄ and R_{b4} are

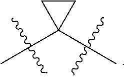 [SMILES: *C1(*)CC1]

In some variations of formula (24) described in the paragraphs above in which l₂ is 4, Rₐ₁ and R_{b1} are H, Rₐ₂ and R_{b2} are H, Rₐ₃ and R_{b3} are CH₃, and Rₐ₄ and R_{b4} are

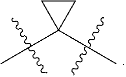 [SMILES: *C1(*)CC1]

In some variations of formula (24) described in the paragraphs above in which l₂ is 4, Rₐ₁ and R_{b1} are H, Rₐ₂ and R_{b2} are CH₃, Rₐ₃ and R_{b3} are CH₃, and Rₐ₄ and R_{b4} are

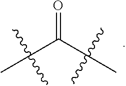 [SMILES: *C(*)=O]

In some variations of formula (24) described in the paragraphs above in which l₂ is 4, Rₐ₁ and R_{b1} are CH₃, Rₐ₂ and R_{b2} are CH₃, Rₐ₃ and R_{b3} are CH₃, and Rₐ₄ and R_{b4} are

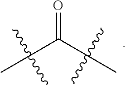 [SMILES: *C(*)=O]

In some variations of formula (24) described in the paragraphs above in which l₂ is 4, Rₐ₁ and R_{b1} are H, Rₐ₂ and R_{b2} are H, Rₐ₃ and R_{b3} are

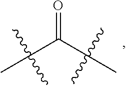 [SMILES: *C(*)=O]

and Rₐ₄ and R_{b4} are v. In some variations of formula (24) described in the paragraphs above in which l₂ is 4, Rₐ₁ and R_{b1} are H, Rₐ₂ and R_{b2} are CH₃, Rₐ₃ and R_{b3} are

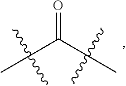 [SMILES: *C(*)=O]

and Rₐ₄ and R_{b4} are

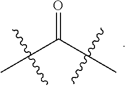 [SMILES: *C(*)=O]

In some variations of formula (24) described in the paragraphs above in which l₂ is 4, Rₐ₁ and R_{b1} are CH₃, Rₐ₂ and R_{b2} are CH₃, Rₐ₃ and R_{b3} are

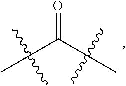 [SMILES: *C(*)=O]

and Rₐ₄ and R_{b4} are

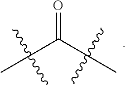 [SMILES: *C(*)=O]

In some variations of formula (24) described in the paragraphs above in which l₂ is 4, Rₐ₁ is H, R_{b1} is CH₃, Rₐ₂ and R_{b2} are CH₃, Rₐ₃ and R_{b3} are

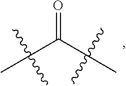 [SMILES: *C(*)=O]

and Rₐ₄ and R_{b4} are

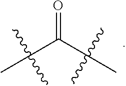 [SMILES: *C(*)=O]

In some variations of formula (24) described in the paragraphs above in which l₂ is 4, Rₐ₁ is H, R_{b1} is CH₃, Rₐ₂ is H, R_{b2} is CH₃, Rₐ₃ and R_{b3} are

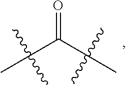 [SMILES: *C(*)=O]

and Rₐ₄ and R_{b4} are

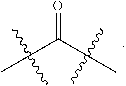 [SMILES: *C(*)=O]

In some variations of formula (24) described in the paragraphs above in which l₂ is 4, Rₐ₁ and R_{b1} are H, Rₐ₂ and R_{b2} are

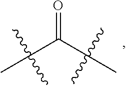 [SMILES: *C(*)=O]

Rₐ₃ and R_{b3} are

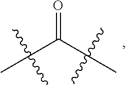 [SMILES: *C(*)=O]

and Rₐ₄ and R_{b4} are

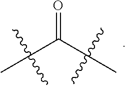 [SMILES: *C(*)=O]

In some variations of formula (24) described in the paragraphs above in which l₂ is 4, Rₐ₁ and R_{b1} are CH₃, Rₐ₂ and R_{b2} are

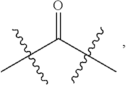 [SMILES: *C(*)=O]

Rₐ₃ and R_{b3} are

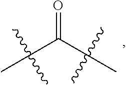 [SMILES: *C(*)=O]

and Rₐ₄ and R_{b4} are

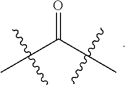 [SMILES: *C(*)=O]

In some variations of formula (24) described in the paragraphs above in which l₂ is 4, Rₐ₁ is H, R_{b1} is CH₃, Rₐ₂ and R_{b2} are

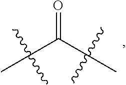 [SMILES: *C(*)=O]

Rₐ₃ and R_{b3} are

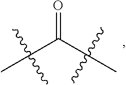 [SMILES: *C(*)=O]

and Rₐ₄ and R_{b4} are

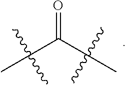 [SMILES: *C(*)=O]

In some variations of formula (24) described in the paragraphs above in which l₂ is 4, Rₐ₁ and R_{b1} are

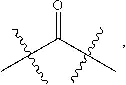 [SMILES: *C(*)=O]

Rₐ₂ and R_{b2} are

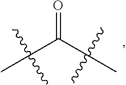 [SMILES: *C(*)=O]

Rₐ₃ and R_{b3} are

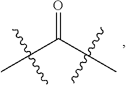 [SMILES: *C(*)=O]

and Rₐ₄ and R_{b4} are

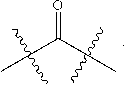 [SMILES: *C(*)=O]

In some variations of formula (24) described in the paragraphs above in which l₂ is 4, Rₐ₁ and R_{b1} are

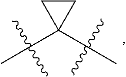 [SMILES: *C1(*)CC1]

Rₐ₂ and R_{b2} are H, Rₐ₃ and R_{b3} are H, and Rₐ₄ and R_{b4} are

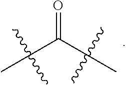 [SMILES: *C(*)=O]

In some variations of formula (24) described in the paragraphs above in which l₂ is 4, Rₐ₁ and R_{b1} are

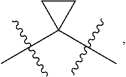 [SMILES: *C1(*)CC1]

Rₐ₂ and R_{b2} are CH₃, Rₐ₃ and R_{b3} are CH₃, and Rₐ₄ and R_{b4} are

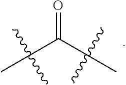 [SMILES: *C(*)=O]

In some variations of formula (24) described in the paragraphs above in which l₂ is 4, Rₐ₁ and R_{b1} are

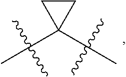 [SMILES: *C1(*)CC1]

Rₐ₂ and R_{b2} are H, Rₐ₃ and R_{b3} are CH₃, and Rₐ₄ and R_{b4} are

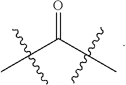 [SMILES: *C(*)=O]

In some variations of formula (24) described in the paragraphs above in which l₂ is 4, Rₐ₁ and R_{b1} are

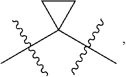 [SMILES: *C1(*)CC1]

Rₐ₂ and R_{b2} are

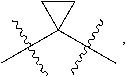 [SMILES: *C1(*)CC1]

Rₐ₃ and R_{b3} are H, and Rₐ₄ and R_{b4} are

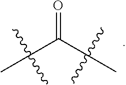 [SMILES: *C(*)=O]

In some variations of formula (24) described in the paragraphs above in which l₂ is 4, Rₐ₁ and R_{b1} are

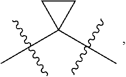 [SMILES: *C1(*)CC1]

Rₐ₂ and R_{b2} are

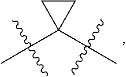 [SMILES: *C1(*)CC1]

Rₐ₃ and R_{b3} are CH₃, and Rₐ₄ and R_{b4} are

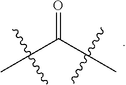 [SMILES: *C(*)=O]

In some variations of formula (24) described in the paragraphs above in which l₂ is 4, Rₐ₁ and R_{b1} are

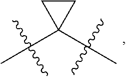 [SMILES: *C1(*)CC1]

Rₐ₂ and R_{b2} are

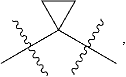 [SMILES: *C1(*)CC1]

Rₐ₃ and R_{b3} are CH₃, and Rₐ₄ and R_{b4} are

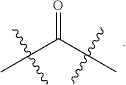 [SMILES: *C(*)=O]

In some variations of formula (24) described in the paragraphs above in which l₂ is 4, Rₐ₁ and R_{b1} are

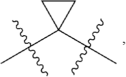 [SMILES: *C1(*)CC1]

Rₐ₂ and R_{b2} are

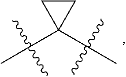 [SMILES: *C1(*)CC1]

Rₐ₃ and R_{b3} are H, and Rₐ₄ and R_{b4} are

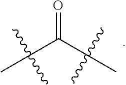 [SMILES: *C(*)=O]

In some variations of formula (24) described in the paragraphs above in which l₂ is 4, Rₐ₁ and R_{b1} are

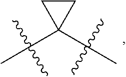 [SMILES: *C1(*)CC1]

Rₐ₂ and R_{b2} are

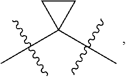 [SMILES: *C1(*)CC1]

Rₐ₃ and R_{b3} are

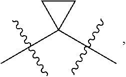 [SMILES: *C1(*)CC1]

and Rₐ₄ and R_{b4} are

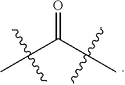 [SMILES: *C(*)=O]

In some variations of formula (24) described in the paragraphs above in which l₂ is 4, Rₐ₁ and R_{b1} are

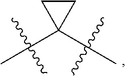 [SMILES: *C1(*)CC1]

Rₐ₂ and R_{b2} are

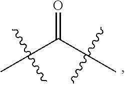 [SMILES: *C(*)=O]

Rₐ₃ and R_{b3} are H, and Rₐ₄ and R_{b4} are

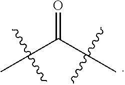 [SMILES: *C(*)=O]

In some variations of formula (24) described in the paragraphs above in which l₂ is 4, Rₐ₁ and R_{b1} are

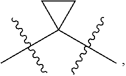 [SMILES: *C1(*)CC1]

Rₐ₂ and R_{b2} are

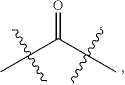 [SMILES: *C(*)=O]

Rₐ₁ and R_{b3} are CH₃, and Rₐ₄ and R_{b4} are

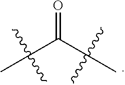 [SMILES: *C(*)=O]

In some variations of formula (24) described in the paragraphs above in which l₂ is 4, Rₐ₁ and R_{b1} are

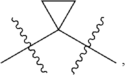 [SMILES: *C1(*)CC1]

Rₐ₂ and R_{b2} are

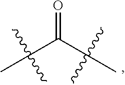 [SMILES: *C(*)=O]

Rₐ₃ and R_{b3} are CH₃, and Rₐ₄ and R_{b4} are

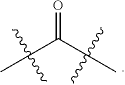 [SMILES: *C(*)=O]

In some variations of formula (24) described in the paragraphs above in which l₂ is 4, Rₐ₁ and R_{b1} are

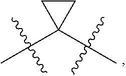 [SMILES: *C1(*)CC1]

Rₐ₂ and R_{b2} are

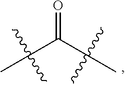 [SMILES: *C(*)=O]

Rₐ₃ and R_{b3} are H, and Rₐ₄ and R_{b4} are

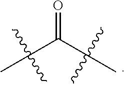 [SMILES: *C(*)=O]

In some variations of formula (24) described in the paragraphs above in which l₂ is 4, Rₐ₁ and R_{b1} are

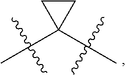 [SMILES: *C1(*)CC1]

Rₐ₂ and R_{b2} are

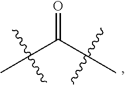 [SMILES: *C(*)=O]

Rₐ₃ and R_{b3} are

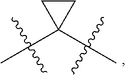 [SMILES: *C1(*)CC1]

and Rₐ₄ and R_{b4} are

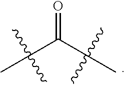 [SMILES: *C(*)=O]

In some variations of formula (24) described in the paragraphs above in which l₂ is 4, Rₐ₁ and R_{b1} are

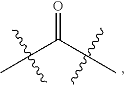 [SMILES: *C(*)=O]

Rₐ₂ and R_{b2} are

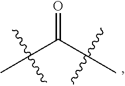 [SMILES: *C(*)=O]

Rₐ₃ and R_{b3} are H, and Rₐ₄ and R_{b4} are

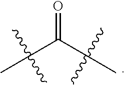 [SMILES: *C(*)=O]

In some variations of formula (24) described in the paragraphs above in which l₂ is 4, Rₐ₁ and R_{b1} are

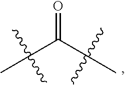 [SMILES: *C(*)=O]

Rₐ₂ and R_{b2} are

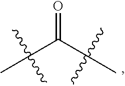 [SMILES: *C(*)=O]

Rₐ₃ and R_{b3} are CH₃, and Rₐ₄ and R_{b4} are

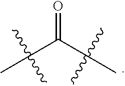 [SMILES: *C(*)=O]

In some variations of formula (24) described in the paragraphs above in which l₂ is 4, Rₐ₁ and R_{b1} are

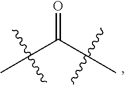 [SMILES: *C(*)=O]

Rₐ₂ and R_{b2} are

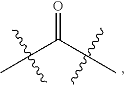 [SMILES: *C(*)=O]

Rₐ₃ and R_{b3} are CH₃, and Rₐ₄ and R_{b4} are

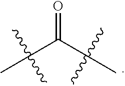 [SMILES: *C(*)=O]

In some variations of formula (24) described in the paragraphs above in which l₂ is 4, Rₐ₁ and R_{b1} are

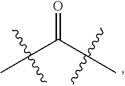 [SMILES: *C(*)=O]

Rₐ₂ and R_{b2} are

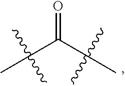 [SMILES: *C(*)=O]

Rₐ₃ and R_{b3} are H, and Rₐ₄ and R_{b4} are

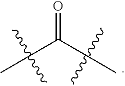 [SMILES: *C(*)=O]

In some variations of formula (24) described in the paragraphs above in which l₂ is 4, Rₐ₁ and R_{b1} are

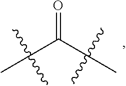 [SMILES: *C(*)=O]

Rₐ₂ and R_{b2} are

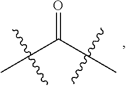 [SMILES: *C(*)=O]

Rₐ₃ and R_{b3} are

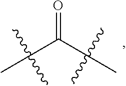 [SMILES: *C(*)=O]

and Rₐ₄ and R_{b4} are

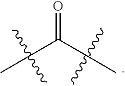 [SMILES: *C(*)=O]

In some variations of formula (24) described in the paragraphs above in which l₂ is 4, Rₐ₁ and R_{b1} are H, Rₐ₂ and R_{b2} are H, and Rₐ₃ and R_{b3} and Rₐ₄ and R_{b4} together are

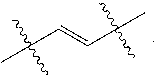 [SMILES: */C=C/*]

In some variations of formula (24) described in the paragraphs above in which l₂ is 4, Rₐ₁ and R_{b1} are H, Rₐ₂ and R_{b2} are CH₃, and Rₐ₃ and R_{b3} and Rₐ₄ and R_{b4} together are

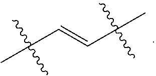 [SMILES: */C=C/*]

In some variations of formula (24) described in the paragraphs above in which l₂ is 4, Rₐ₁ and Rₐ₂ are H, R_{b1} and R_{b2} are CH₃, and Rₐ₃ and R_{b3} and Rₐ₄ and R_{b4} together are

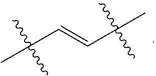 [SMILES: */C=C/*]

In some variations of formula (24) described in the paragraphs above in which l₂ is 4, Rₐ₁ and R_{b1} are H, Rₐ₂ and R_{b2} are

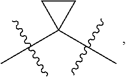 [SMILES: *C1(*)CC1]

and Rₐ₃ and R_{b3} and Rₐ₄ and R_{b4} together are

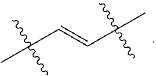 [SMILES: */C=C/*]

In some variations of formula (24) described in the paragraphs above in which l₂ is 4, Rₐ₁ and R_{b1} are

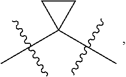 [SMILES: *C1(*)CC1]

Rₐ₂ and R_{b2} are CH₃, and Rₐ₃ and R_{b3} and Rₐ₄ and R_{b4} together are

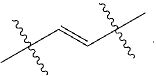 [SMILES: */C=C/*]

In some variations of formula (24) described in the paragraphs above in which l₂ is 4, Rₐ₁ is H, R_{b1} is CH₃, Rₐ₂ and R_{b2} are

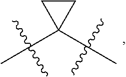 [SMILES: *C1(*)CC1]

and Rₐ₃ and R_{b3} and Rₐ₄ and R_{b4} together are

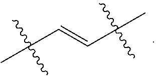 [SMILES: */C=C/*]

In some variations of formula (24) described in the paragraphs above in which l₂ is 4, Rₐ₁ and R_{b1} are H, Rₐ₂ and R_{b2} are

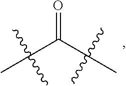 [SMILES: *C(*)=O]

and Rₐ₃ and R_{b3} and Rₐ₄ and R_{b4} together are

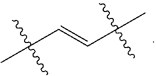 [SMILES: */C=C/*]

In some variations of formula (24) described in the paragraphs above in which l₂ is 4, Rₐ₁ and R_{b1} are

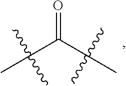 [SMILES: *C(*)=O]

Rₐ₂ and R_{b2} are CH₃, and Rₐ₃ and R_{b3} and Rₐ₄ and R_{b4} together are

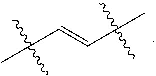 [SMILES: */C=C/*]

In some variations of formula (24) described in the paragraphs above in which l₂ is 4, Rₐ₁ is H, R_{b1} is CH₃, Rₐ₂ and R_{b2} are

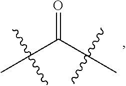 [SMILES: *C(*)=O]

and Rₐ₃ and R_{b3} and Rₐ₄ and R_{b4} together are

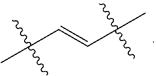 [SMILES: */C=C/*]

In some variations of formula (24) described in the paragraphs above in which l₂ is 4, Rₐ₁ and R_{b1} are

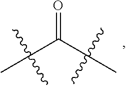 [SMILES: *C(*)=O]

Rₐ₂ and R_{b2} are

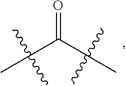 [SMILES: *C(*)=O]

and Rₐ₃ and R_{b3} and Rₐ₄ and R_{b4} together are

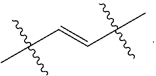 [SMILES: */C=C/*]

In some variations of formula (24) described in the paragraphs above in which l₂ is 4, Rₐ₁ and R_{b1} are

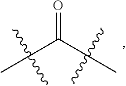 [SMILES: *C(*)=O]

Rₐ₂ and R_{b2} are

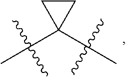 [SMILES: *C1(*)CC1]

and Rₐ₃ and R_{b3} and Rₐ₄ and R_{b4} together are

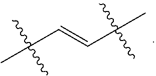 [SMILES: */C=C/*]

In some variations of formula (24) described in the paragraphs above in which l₂ is 4, Rₐ₁ and R_{b1} are

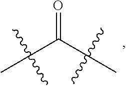 [SMILES: *C(*)=O]

Rₐ₂ and R_{b2} are

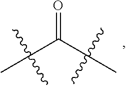 [SMILES: *C(*)=O]

and Rₐ₃ and R_{b3} and Rₐ₄ and R_{b4} together are

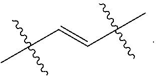 [SMILES: */C=C/*]

In some variations of formula (24) described in the paragraphs above in which l₂ is 4, Rₐ₁ and R_{b1} are

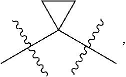 [SMILES: *C1(*)CC1]

Rₐ₂ and R_{b2} are

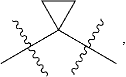 [SMILES: *C1(*)CC1]

and Rₐ₃ and R_{b3} and Rₐ₄ and R_{b4} together are

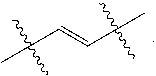 [SMILES: */C=C/*]

In some variations of formula (24) described in the paragraphs above in which l₂ is 4, Rₐ₁ and R_{b1} and Rₐ₂ and R_{b2} together are

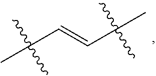 [SMILES: */C=C/*]

and Rₐ₃ and R_{b3} and Rₐ₄ and R_{b4} together are

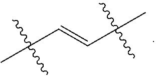 [SMILES: */C=C/*]

In some variations of formula (24) described in the paragraphs above, ring A is

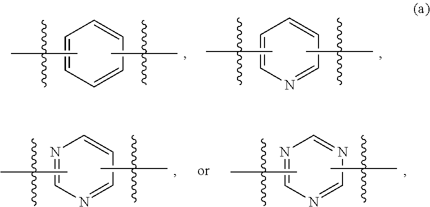

optionally substituted with halo or C1-C6 linear or branched alkyl;

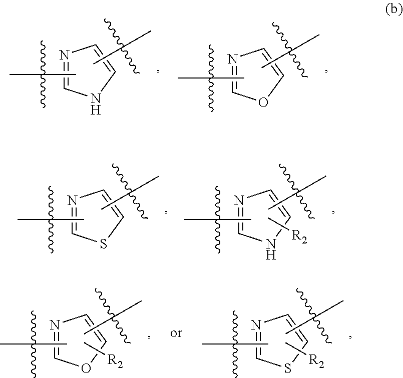

optionally substituted with halo or C1-C6 linear or branched alkyl; or

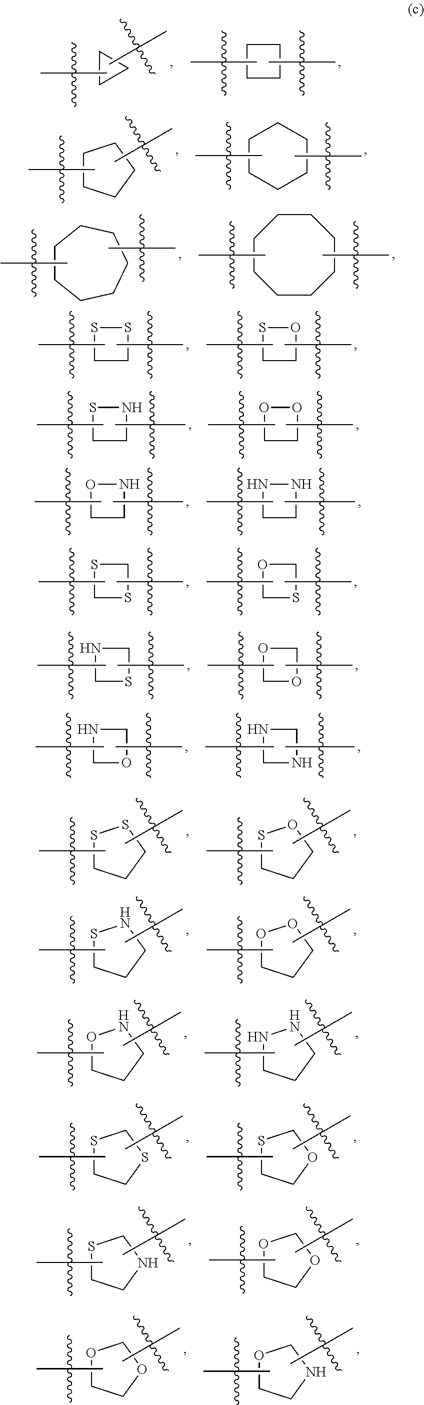

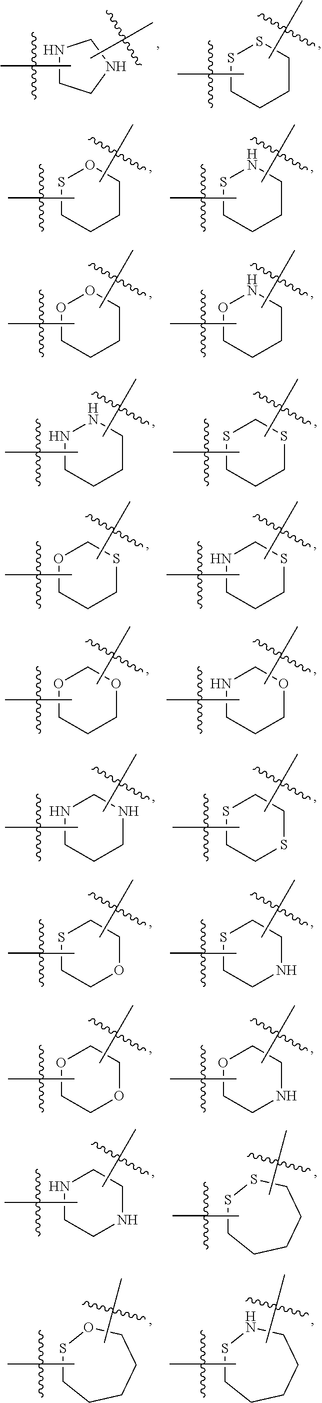

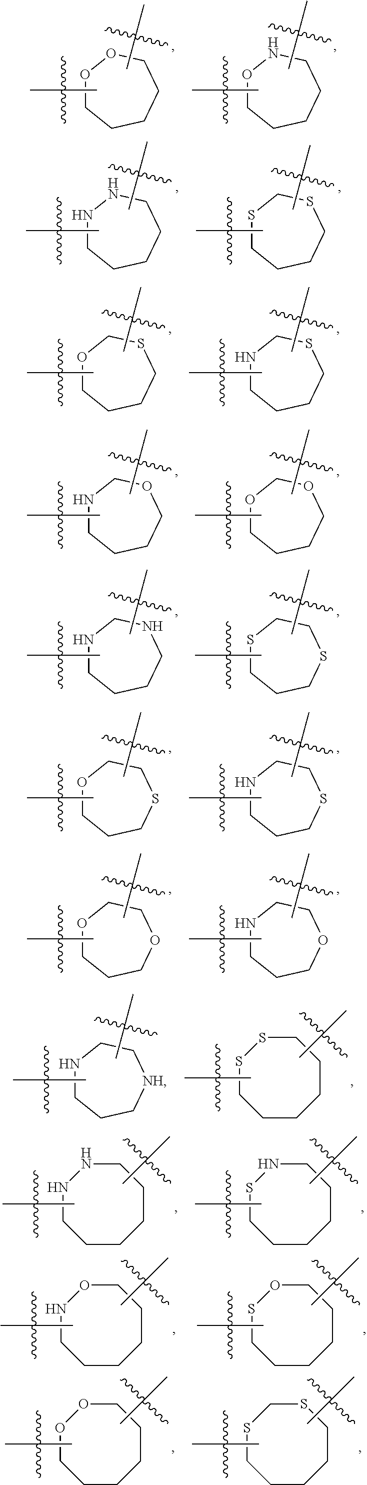

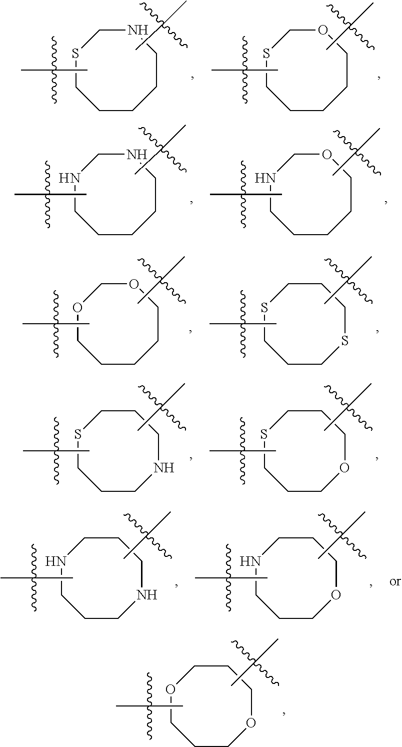

optionally substituted with halo or C1-C6 linear or branched alkyl.

In some variations of formula (24) described in the paragraphs above, ring A is substituted with halo. In some variations, the halo is F, Br, I, or Cl.

In some variations of formula (24) described in the paragraphs above, ring A is substituted with C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (24) described in the paragraphs above, D1 is safranin-O. In some variations of formula (24) described in the paragraphs above, the first D2 is safranin-O. In some variations of formula (24) described in the paragraphs above, the second D2 is safranin-O. In some variations of formula (24) described in the paragraphs above, D1 and the first D2 are safranin-O. In some variations of formula (24) described in the paragraphs above, D1 and the second D2 are safranin-O. In some variations of formula (24) described in the paragraphs above, the first D2 and the second D2 are safranin-O.

In some variations of formula (24) described in the paragraph above, the pendant phenyl ring of D1 is unsubstituted. In some variations of formula (24) described in the paragraph above, the pendant phenyl ring of D1 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D1 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D1 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (24) described in the two paragraphs above, the pendant phenyl ring of the first D2 is unsubstituted. In some variations of formula (24) described in the paragraph above, the pendant phenyl ring of the first D2 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of the first D2 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of the first D2 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (24) described in the three paragraphs above, the pendant phenyl ring of the second D2 is unsubstituted. In some variations of formula (24) described in the paragraph above, the pendant phenyl ring of the second D2 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of the second D2 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of the second D2 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some of the variations of formula (24) described above, in which each D is a safranin-O moiety, as shown in formula (24a):

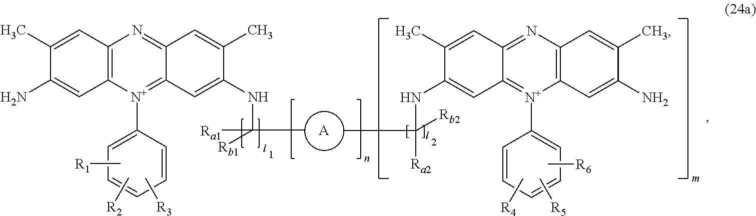

in which m, l₁, l₂, ring A, Rₐ₁, R_{b1} Rₐ₂, and R_{b2} are as defined in the paragraphs above, R₁, R₂, R₃, R₄, R₅, and R₆ independently are absent or independently are selected from —NH₂, —NHR, —NR₂, —OH, —O⁻, —NHCOCH₃, —NHCOR, —OCH₃, —OR, —C₂H₅, —R, —C₆H₅, —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR), and R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

One of skill in the art can readily visualize and prepare other cationic multimers falling within formula (24) in which other cationic dye moieties are used in place of one or both of the safranin-O moieties.

Some cationic dye multimers fall within formula (25):

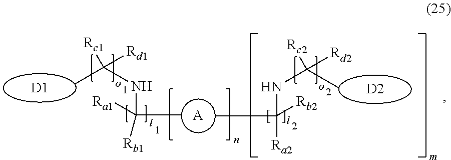

in which m is 2; each of D1, a first D2, and a second D2 is a cationic dye moiety; l₁, l₂, n, o₁ and o₂ independently are 1-4; ring A is aryl, heteroaryl, cycloalkyl, or heterocyclyl; for each independent instance of Rₐ₁ and R_{b1}, Rₐ₁ and R_{b1} (1) independently are H or CH₃, or (2) Rₐ₁ and R_{b1} independently are

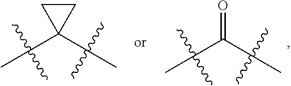

or (3) two of CRₐ₁R_{b1} are

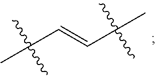 [SMILES: */C=C/*]

for each independent instance of Rₐ₂ and R_{b2}, Rₐ₂ and R_{b2} (1) independently are H or CH₃, or (2) Rₐ₂ and R_{b2} independently are

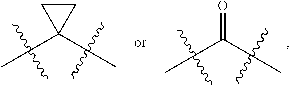

or (3) two of CRₐ₂R_{b2} are

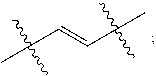 [SMILES: */C=C/*]

for each independent instance of R_{c1} and R_{d1}, R_{c1} and R_{d1} (1) independently are H or CH₃, or (2) R_{c1} and R_{d1} independently are

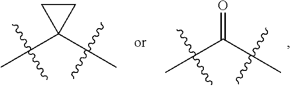

or (3) two of CR_{c1}R_{d1} are

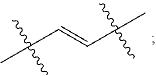 [SMILES: */C=C/*]

and, for each independent instance of R_{c2} and R_{d2}, R_{c2} and R_{d2} (1) independently are H or CH₃, or (2) Rₑₑ and R_{d2} independently are

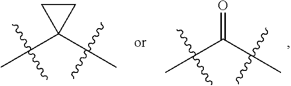

or (3) two of CR_{c2}R_{d2} are

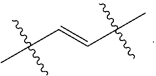 [SMILES: */C=C/*]

In some variations of formula (25), D1, the first D2, and the second D2 are different cationic dye moieties. In some variations of formula (25), D1, the first D2, and the second D2 are the same cationic dye moiety. In other variations of formula (25), D1 and the first D2 are the same cationic dye moiety and the second D2 is a different cationic dye moiety. In other variations of formula (25), D1 and the second D2 are the same cationic dye moiety and the first D2 is a different cationic dye moiety. In other variations of formula (25), the first D2 and the second D2 are the same cationic dye moiety and D1 is a different cationic dye moiety. In some variations of formula (25), each of D1, the first D2, and the second D2 independently is selected from the group consisting of safranin-O, toluidine blue, azure A, azure B, azure C, acridine orange, acriflavine, and methylene blue.

In some variations of formula (25) described in the paragraphs above, l₁ is 1-4, 1-3, 1-2, 2-4, 2-3, 3-4, 1, 2, 3, or 4.

In some variations of formula (25) described in the paragraphs above, l₂ is 1-4, 1-3, 1-2, 2-4, 2-3, 3-4, 1, 2, 3, or 4.

In some previous variations of formula (25) described in the paragraphs above, o₁ is 1-4, 1-3, 1-2, 2-4, 2-3, 3-4, 1, 2, 3, or 4.

In some previous variations of formula (25) described in the paragraphs above, o₂ is 1-4, 1-3, 1-2, 2-4, 2-3, 3-4, 1, 2, 3, or 4.

In some variations of formula (25) described in the paragraphs above in which l₁ is 1, Rₐ₁ and R_{b1} are both H. In some variations of formula (25) described in the paragraphs above in which l₁ is 1, Rₐ₁ is H and R_{b1} is CH₃. In some variations of formula (25) described in the paragraphs above in which l₁ is 1, Rₐ₁ and R_{b1} are both CH₃. In some variations of formula (25) described in the paragraphs above in which l₁ is 1, Rₐ₁ and R_{b1} are

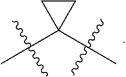 [SMILES: *C1(*)CC1]

In some variations of formula (25) described in the paragraphs above in which l₁ is 1, Rₐ₁ and R_{b1} are

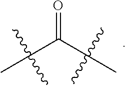 [SMILES: *C(*)=O]

In variations of formula (25) described in the paragraphs above in which l₁ is 2, the two instances of Rₐ and R_{b} are indicated as Rₐ₁ and R_{b1} and Rₐ₂ and R_{b2}, respectively. In some variations of formula (25) described in the paragraphs above in which l₁ is 2, each of Rₐ₁ and R_{b1} and Rₐ₂ and R_{b2} is H. In some variations of formula (25) described in the paragraphs above in which l₁ is 2, each of Rₐ₁ and R_{b1} and Rₐ₂ and R_{b2} is CH₃. In some variations of formula (25) described in the paragraphs above in which l₁ is 2, each of Rₐ₁ and Rₐ₂ is H and each of R_{b1} and R_{b2} is CH₃. In some variations of formula (25) described in the paragraphs above in which l₁ is 2, each of Rₐ₁, Rₐ₂, and R_{b1} is H and R_{b2} is CH₃.

In some variations of formula (25) described in the paragraphs above in which l₁ is 2, each of Rₐ₁ and R_{b1} is H and Rₐ₂ and R_{b2} are

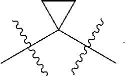 [SMILES: *C1(*)CC1]

In some variations of formula (25) described in the paragraphs above in which l₁ is 2, each of Rₐ₁ and R_{b1} is H and Rₐ₂ and R_{b2} are

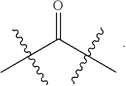 [SMILES: *C(*)=O]

In some variations of formula (25) described in the paragraphs above in which l₁ is 2, each of Rₐ₁ and R_{b1} is CH₃ and Rₐ₂ and R_{b2} are

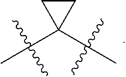 [SMILES: *C1(*)CC1]

In some variations of formula (25) described in the paragraphs above in which l₁ is 2, each of Rₐ₁ and R_{b1} is CH₃ and Rₐ₂ and R_{b2} are

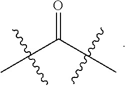 [SMILES: *C(*)=O]

In some variations of formula (25) described in the paragraphs above in which l₁ is 2, Rₐ₁ is H, R_{b1} is CH₃, and Rₐ₂ and R_{b2} are

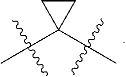 [SMILES: *C1(*)CC1]

In some variations of formula (25) described in the paragraphs above in which l₁ is 2, Rₐ₁ is H, R_{b1} is CH₃, and Rₐ₂ and R_{b2} are

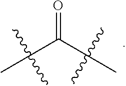 [SMILES: *C(*)=O]

In some variations of formula (25) described in the paragraphs above in which l₁ is 2, Rₐ₁ and R_{b1} and Rₐ₂ and R_{b2} together are

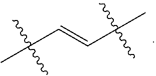 [SMILES: */C=C/*]

In some variations of formula (25) described in the paragraphs above in which l₁ is 2, Rₐ₁ and R_{b1} are

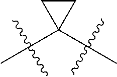 [SMILES: *C1(*)CC1]

Rₐ₂ and R_{b2} are

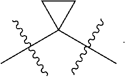 [SMILES: *C1(*)CC1]

In some variations of formula (25) described in the paragraphs above in which l₁ is 2, Rₐ₁ and R_{b1} are

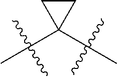 [SMILES: *C1(*)CC1]

Rₐ₂ and R_{b2} are

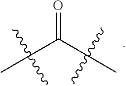 [SMILES: *C(*)=O]

In some variations of formula (25) described in the paragraphs above in which l₁ is 2, Rₐ₁ and R_{b1} are

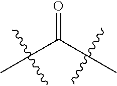 [SMILES: *C(*)=O]

Rₐ₂ and R_{b2} are

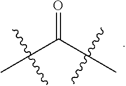 [SMILES: *C(*)=O]

In variations of formula (25) described in the paragraphs above in which l₁ is 3, the three instances of Rₐ and R_{b} are indicated as Rₐ₁ and R_{b1}; Rₐ₂ and R_{b2}; and Rₐ₃ and R_{b3}, respectively. In some variations of formula (25) described in the paragraphs above in which l₁ is 3, In some variations of formula (25) described in the paragraphs above in which l₁ is 3, each of Rₐ₁, R_{b1}, Rₐ₂, R_{b2}, Rₐ₃, and R_{b3} is H. In some variations of formula (25) described in the paragraphs above in which l₁ is 3, each of Rₐ₁, R_{b1}, Rₐ₂, R_{b2}, Rₐ₃, and R_{b3} is CH₃. In some variations of formula (25) described in the paragraphs above in which l₁ is 3, each of Rₐ₁, R_{b1}, Rₐ₂, and R_{b2}, is H and each of Rₐ₃ and R_{b3} is CH₃. In some variations of formula (25) described in the paragraphs above in which l₁ is 3, each of Rₐ₁, R_{b1}, Rₐ₂, and R_{b2}, is CH₃ and each of Rₐ₃ and R_{b3} is H. In some variations of formula (25) described in the paragraphs above in which l₁ is 3, Rₐ₁ is H and each of Rₐ₂, Rₐ₃, R_{b1}, R_{b2}, and R_{b3} is CH₃. In some variations of formula (25) described in the paragraphs above in which l₁ is 3, each of Rₐ₁ and Rₐ₂ is H and each of Rₐ₃, R_{b1}, R_{b2}, an R_{b3} is CH₃. In some variations of formula (25) described in the paragraphs above in which l₁ is 3, each of Rₐ₁, Rₐ₂, and Rₐ₃ is H and each of R_{b1}, R_{b2}, an R_{b3} is CH₃.

In some variations of formula (25) described in the paragraphs above in which l₁ is 3, each of Rₐ₁, R_{b1}, Rₐ₂, and R_{b2}, is H and Rₐ₃ and R_{b3} are

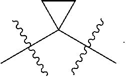 [SMILES: *C1(*)CC1]

In some variations of formula (25) described in the paragraphs above in which l₁ is 3, each of Rₐ₁, R_{b1}, Rₐ₂, and R_{b2}, is CH₃ and Rₐ₃ and R_{b3} are

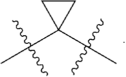 [SMILES: *C1(*)CC1]

In some variations of formula (25) described in the paragraphs above in which l₁ is 3, Rₐ₁ is H and each of Rₐ₂, R_{b1}, and R_{b2} is CH₃, and Rₐ₃ and R_{b3} are

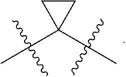 [SMILES: *C1(*)CC1]

In some variations of formula (25) described in the paragraphs above in which l₁ is 3, each of Rₐ₁ and Rₐ₂ is H and each of R_{b1} and R_{b2} is CH₃, and Rₐ₃ and R_{b3} are

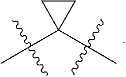 [SMILES: *C1(*)CC1]

In some variations of formula (25) described in the paragraphs above in which l₁ is 3, each of Rₐ₁, R_{b1}, Rₐ₂, and R_{b2}, is H and Rₐ₃ and R_{b3} are

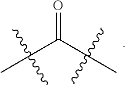 [SMILES: *C(*)=O]

In some variations of formula (25) described in the paragraphs above in which l₁ is 3, each of Rₐ₁, R_{b1}, Rₐ₂, and R_{b2}, is CH₃ and Rₐ₃ and R_{b3} are

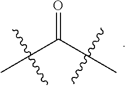 [SMILES: *C(*)=O]

In some variations of formula (25) described in the paragraphs above in which l₁ is 3, Rₐ₁ is H and each of Rₐ₂, R_{b1}, and R_{b2} is CH₃, and Rₐ₃ and R_{b3} are

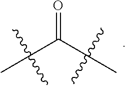 [SMILES: *C(*)=O]

In some variations of formula (25) described in the paragraphs above in which l₁ is 3, each of Rₐ₁ and Rₐ₂ is H and each of R_{b1} and R_{b2} is CH₃, and Rₐ₃ and R_{b3} are

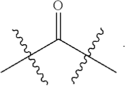 [SMILES: *C(*)=O]

In some variations of formula (25) described in the paragraphs above in which l₁ is 3, Rₐ₁ and R_{b1} are

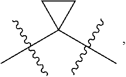 [SMILES: *C1(*)CC1]

Rₐ₂ and R_{b2} are

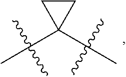 [SMILES: *C1(*)CC1]

and each of Rₐ₃ and R_{b3} is H. In some variations of formula (25) described in the paragraphs above in which l₁ is 3, Rₐ₁ and R_{b1} are

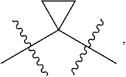 [SMILES: *C1(*)CC1]

Rₐ₂ and R_{b2} are

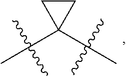 [SMILES: *C1(*)CC1]

and each of Rₐ₃ and R_{b3} is CH₃. In some variations of formula (25) described in the paragraphs above in which l₁ is 3, Rₐ₁ and R_{b1} are

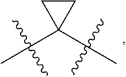 [SMILES: *C1(*)CC1]

Rₐ₂ and R_{b2} are

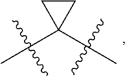 [SMILES: *C1(*)CC1]

Rₐ₃ is H, and R_{b3} is CH₃. In some variations of formula (25) described in the paragraphs above in which l₁ is 3, Rₐ₁ and R_{b1} are

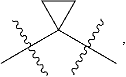 [SMILES: *C1(*)CC1]

Rₐ₂ and R_{b2} are

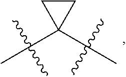 [SMILES: *C1(*)CC1]

and each of Rₐ₃ and R_{b3} is H. In some variations of formula (25) described in the paragraphs above in which l₁ is 3, Rₐ₁ and R_{b1} are

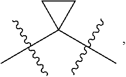 [SMILES: *C1(*)CC1]

Rₐ₂ and R_{b2} are

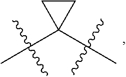 [SMILES: *C1(*)CC1]

and each of Rₐ₃ and R_{b3} is CH₃. In some variations of formula (25) described in the paragraphs above in which l₁ is 3, Rₐ₁ and R_{b1} are

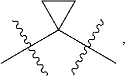 [SMILES: *C1(*)CC1]

Rₐ₂ and R_{b2} are

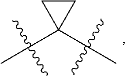 [SMILES: *C1(*)CC1]

Rₐ₃ is H, and R_{b3} is CH₃.

In some variations of formula (25) described in the paragraphs above in which l₁ is 3, Rₐ₁ and R_{b1} are

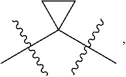 [SMILES: *C1(*)CC1]

Rₐ₂ and R_{b2} are

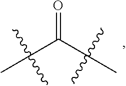 [SMILES: *C(*)=O]

and each of Rₐ₃ and R_{b3} is H. In some variations of formula (25) described in the paragraphs above in which l₁ is 3, Rₐ₁ and R_{b1} are

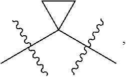 [SMILES: *C1(*)CC1]

Rₐ₂ and R_{b2} are

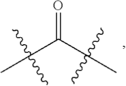 [SMILES: *C(*)=O]

and each of Rₐ₃ and R_{b3} is CH₃. In some variations of formula (25) described in the paragraphs above in which l₁ is 3, Rₐ₁ and R_{b1} are

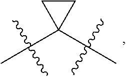 [SMILES: *C1(*)CC1]

Rₐ₂ and R_{b2} are

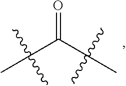 [SMILES: *C(*)=O]

Rₐ₃ is H, and R_{b3} is CH₃. In some variations of formula (25) described in the paragraphs above in which l₁ is 3, Rₐ₁ and R_{b1} are

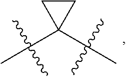 [SMILES: *C1(*)CC1]

Rₐ₂ and R_{b2} are

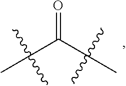 [SMILES: *C(*)=O]

and each of Rₐ₃ and R_{b3} is H. In some variations of formula (25) described in the paragraphs above in which l₁ is 3, Rₐ₁ and R_{b1} are

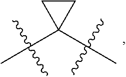 [SMILES: *C1(*)CC1]

Rₐ₂ and R_{b2} are

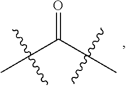 [SMILES: *C(*)=O]

and each of Rₐ₃ and R_{b3} is CH₃. In some variations of formula (25) described in the paragraphs above in which l₁ is 3, Rₐ₁ and R_{b1} are

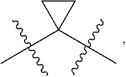 [SMILES: *C1(*)CC1]

Rₐ₂ and R_{b2} are

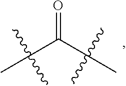 [SMILES: *C(*)=O]

Rₐ₃ is H, and R_{b3} is CH₃.

In some variations of formula (25) described in the paragraphs above in which l₁ is 3, Rₐ₁ and R_{b1} are

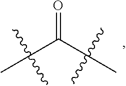 [SMILES: *C(*)=O]

Rₐ₂ and R_{b2} are

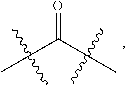 [SMILES: *C(*)=O]

and each of Rₐ₃ and R_{b3} is H. In some variations of formula (25) described in the paragraphs above in which l₁ is 3, Rₐ₁ and R_{b1} are

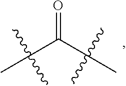 [SMILES: *C(*)=O]

Rₐ₂ and R_{b2} are

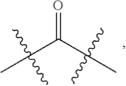 [SMILES: *C(*)=O]

and each of Rₐ₃ and R_{b3} is CH₃. In some variations of formula (25) described in the paragraphs above in which l₁ is 3, Rₐ₁ and R_{b1} are

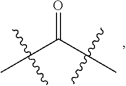 [SMILES: *C(*)=O]

Rₐ₂ and R_{b2} are

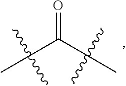 [SMILES: *C(*)=O]

Rₐ₃ is H, and R_{b3} is CH₃. In some variations of formula (25) described in the paragraphs above in which l₁ is 3, Rₐ₁ and R_{b1} are

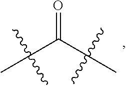 [SMILES: *C(*)=O]

Rₐ₂ and R_{b2} are

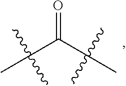 [SMILES: *C(*)=O]

and each of Rₐ₃ and R_{b3} is H. In some variations of formula (25) described in the paragraphs above in which l₁ is 3, Rₐ₁ and R_{b1} are

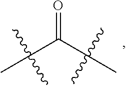 [SMILES: *C(*)=O]

Rₐ₂ and R_{b2} are

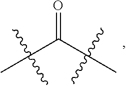 [SMILES: *C(*)=O]

and each of Rₐ₃ and R_{b3} is CH₃. In some variations of formula (25) described in the paragraphs above in which l₁ is 3, Rₐ₁ and R_{b1} are

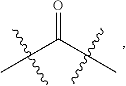 [SMILES: *C(*)=O]

Rₐ₂ and R_{b2} are

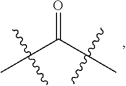 [SMILES: *C(*)=O]

Rₐ₃ is H, and R_{b3} is CH₃.

In some variations of formula (25) described in the paragraphs above in which l₁ is 3, Rₐ₁ and R_{b1} and Rₐ₂ and R_{b2} together are

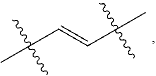 [SMILES: */C=C/*]

and each of Rₐ₃ and R_{b3} is H. In some variations of formula (25) described in the paragraphs above in which l₁ is 3, Rₐ₁ and R_{b1} and Rₐ₂ and R_{b2} together are

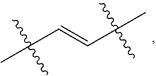 [SMILES: */C=C/*]

and each of Rₐ₃ and R_{b3} is CH₃. In some variations of formula (25) described in the paragraphs above in which l₁ is 3, Rₐ₁ and R_{b1} and Rₐ₂ and R_{b2} together are

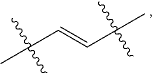 [SMILES: */C=C/*]

Rₐ₃ is H, and R_{b3} is CH₃. In some variations of formula (25) described in the paragraphs above in which l₁ is 3, Rₐ₁ and R_{b1} and Rₐ₂ and R_{b2} together are

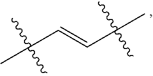 [SMILES: */C=C/*]

and Rₐ₃ and R_{b3} are

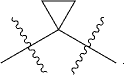 [SMILES: *C1(*)CC1]

In some variations of formula (25) described in the paragraphs above in which l₁ is 3, Rₐ₁ and R_{b1} and Rₐ₂ and R_{b2} together are

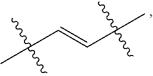 [SMILES: */C=C/*]

and Rₐ₃ and R_{b3} are

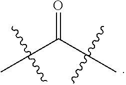 [SMILES: *C(*)=O]

In variations of formula (25) described in the paragraphs above in which l₁ is 4, the three instances of Rₐ and R_{b} are indicated as Rₐ₁ and R_{b1}; Rₐ₂ and R_{b2}; Rₐ₃ and R_{b3}; and Rₐ₄ and R_{b4}, respectively. In some variations of formula (25) described in the paragraphs above in which l₁ is 4, In some variations of formula (25) described in the paragraphs above in which l₁ is 4, each of Rₐ₁, R_{b1}, Rₐ₂, R_{b2}, Rₐ₃, R_{b3}, Rₐ₄, and R_{b4} is H. In some variations of formula (25) described in the paragraphs above in which l₁ is 4, each of Rₐ₁, R_{b1}, Rₐ₂, R_{b2}, Rₐ₃, R_{b3}, Rₐ₄, and R_{b4} is CH₃. In some variations of formula (25) described in the paragraphs above in which l₁ is 4, each of Rₐ₁, R_{b1}, Rₐ₂, R_{b2}, Rₐ₃, and R_{b3} is H and each of Rₐ₄ and R_{b4} is CH₃. In some variations of formula (25) described in the paragraphs above in which l₁ is 4, each of Rₐ₁, R_{b1}, Rₐ₂, and R_{b2} is H and each of Rₐ₃, R_{b3}, Rₐ₄, and R_{b4} is CH₃. In some variations of formula (25) described in the paragraphs above in which l₁ is 4, each of Rₐ₁, R_{b1}, Rₐ₂, R_{b2}, Rₐ₃, and R_{b3} is CH₃ and each of Rₐ₄ and R_{b4} is H.

In some variations of formula (25) described in the paragraphs above in which l₁ is 4, each of Rₐ₁, Rₐ₂, and Rₐ₃ is H and each of R_{b1}, R_{b2}, and R_{b3} is CH₃. In some variations of formula (25) described in the paragraphs above in which l₁ is 4, Rₐ₁ is H, R_{b1} is CH₃, and each of Rₐ₂, R_{b2}, Rₐ₃, and R_{b3} is CH₃. In some variations of formula (25) described in the paragraphs above in which l₁ is 4, Rₐ₁ is H, R_{b1} is CH₃, and each of Rₐ₂, R_{b2}, Rₐ₃, and R_{b3} is H. In some variations of formula (25) described in the paragraphs above in which l₁ is 4, each of Rₐ₁ and Rₐ₂ is H, each of R_{b1} and R_{b2} is CH₃, and each of Rₐ₃ and R_{b3} is H. In some variations of formula (25) described in the paragraphs above in which l₁ is 4, each of Rₐ₁ and Rₐ₂ is H, each of R_{b1} and R_{b2} is CH₃, and each of Rₐ₃ and R_{b3} is CH₃.

In some variations of formula (25) described in the paragraphs above in which l₁ is 4, Rₐ₁ and R_{b1} are H, Rₐ₂ and R_{b2} are H, Rₐ₃ and R_{b3} are H, and Rₐ₄ and R_{b4} are

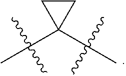 [SMILES: *C1(*)CC1]

In some variations of formula (25) described in the paragraphs above in which l₁ is 4, Rₐ₁ and R_{b1} are H, Rₐ₂ and R_{b2} are H, Rₐ₃ and R_{b3} are CH₃, and Rₐ₄ and R_{b4} are

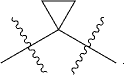 [SMILES: *C1(*)CC1]

In some variations of formula (25) described in the paragraphs above in which l₁ is 4, Rₐ₁ and R_{b1} are H, Rₐ₂ and R_{b2} are CH₃, Rₐ₃ and R_{b3} are CH₃, and Rₐ₄ and R_{b4} are

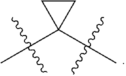 [SMILES: *C1(*)CC1]

In some variations of formula (25) described in the paragraphs above in which l₁ is 4, Rₐ₁ and R_{b1} are CH₃, Rₐ₂ and R_{b2} are CH₃, Rₐ₃ and R_{b3} are CH₃, and Rₐ₄ and R_{b4} are

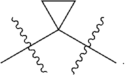 [SMILES: *C1(*)CC1]

In some variations of formula (25) described in the paragraphs above in which l₁ is 4, Rₐ₁ and R_{b1} are H, Rₐ₂ and R_{b2} are H, Rₐ₃ and R_{b3} are

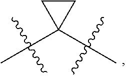 [SMILES: *C1(*)CC1]

and Rₐ₄ and R_{b4} are

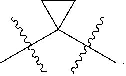 [SMILES: *C1(*)CC1]

In some variations of formula (25) described in the paragraphs above in which l₁ is 4, Rₐ₁ and R_{b1} are H, Rₐ₂ and R_{b2} are CH₃, Rₐ₃ and R_{b3} are

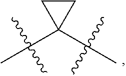 [SMILES: *C1(*)CC1]

and Rₐ₄ and R_{b4} are

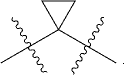 [SMILES: *C1(*)CC1]

In some variations of formula (25) described in the paragraphs above in which l₁ is 4, Rₐ₁ and R_{b1} are CH₃, Rₐ₂ and R_{b2} are CH₃, Rₐ₃ and R_{b3} are

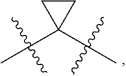 [SMILES: *C1(*)CC1]

and Rₐ₄ and R_{b4} are

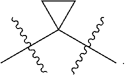 [SMILES: *C1(*)CC1]

In some variations of formula (25) described in the paragraphs above in which l₁ is 4, Rₐ₁ is H, R_{b1} is CH₃, Rₐ₂ and R_{b2} are CH₃, Rₐ₃ and R_{b3} are

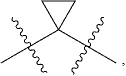 [SMILES: *C1(*)CC1]

and Rₐ₄ and R_{b4} are

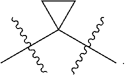 [SMILES: *C1(*)CC1]

In some variations of formula (25) described in the paragraphs above in which l₁ is 4, Rₐ₁ is H, R_{b1} is CH₃, Rₐ₂ is H, R_{b2} is CH₃, Rₐ₃ and R_{b3} are

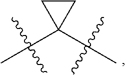 [SMILES: *C1(*)CC1]

and Rₐ₄ and R_{b4} are

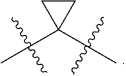 [SMILES: *C1(*)CC1]

In some variations of formula (25) described in the paragraphs above in which l₁ is 4, Rₐ₁ and R_{b1} are H, Rₐ₂ and R_{b2} are

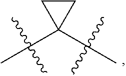 [SMILES: *C1(*)CC1]

Rₐ₃ and R_{b3} are

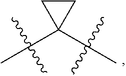 [SMILES: *C1(*)CC1]

and Rₐ₄ and R_{b4} are

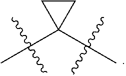 [SMILES: *C1(*)CC1]

In some variations of formula (25) described in the paragraphs above in which l₁ is 4, Rₐ₁ and R_{b1} are CH₃, Rₐ₂ and R_{b2} are

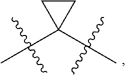 [SMILES: *C1(*)CC1]

Rₐ₃ and R_{b3} are

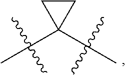 [SMILES: *C1(*)CC1]

and Rₐ₄ and R_{b4} are

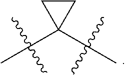 [SMILES: *C1(*)CC1]

In some variations of formula (25) described in the paragraphs above in which l₁ is 4, Rₐ₁ is H, R_{b1} is CH₃, Rₐ₂ and R_{b2} are

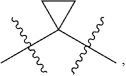 [SMILES: *C1(*)CC1]

Rₐ₃ and R_{b3} are

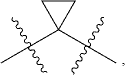 [SMILES: *C1(*)CC1]

and Rₐ₄ and R_{b4} are

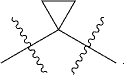 [SMILES: *C1(*)CC1]

In some variations of formula (25) described in the paragraphs above in which l₁ is 4, Rₐ₁ and R_{b1} are

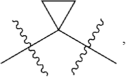 [SMILES: *C1(*)CC1]

Rₐ₂ and R_{b2} are

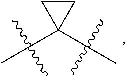 [SMILES: *C1(*)CC1]

Rₐ₃ and R_{b3} are

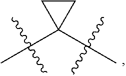 [SMILES: *C1(*)CC1]

and Rₐ₄ and R_{b4} are

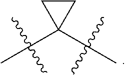 [SMILES: *C1(*)CC1]

In some variations of formula (25) described in the paragraphs above in which l₁ is 4, Rₐ₁ and R_{b1} are H, Rₐ₂ and R_{b2} are H, Rₐ₃ and R_{b3} are H, and Rₐ₄ and R_{b4} are

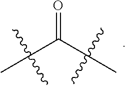 [SMILES: *C(*)=O]

In some variations of formula (25) described in the paragraphs above in which l₁ is 4, Rₐ₁ and R_{b1} are H, Rₐ₂ and R_{b2} are H, Rₐ₃ and R_{b3} are CH₃, and Rₐ₄ and R_{b4} are

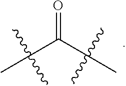 [SMILES: *C(*)=O]

In some variations of formula (25) described in the paragraphs above in which l₁ is 4, Rₐ₁ and R_{b1} are H, Rₐ₂ and R_{b2} are CH₃, Rₐ₃ and R_{b3} are CH₃, and Rₐ₄ and R_{b4} are

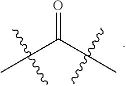 [SMILES: *C(*)=O]

In some variations of formula (25) described in the paragraphs above in which l₁ is 4, Rₐ₁ and R_{b1} are CH₃, Rₐ₂ and R_{b2} are CH₃, Rₐ₃ and R_{b3} are CH₃, and Rₐ₄ and R_{b4} are

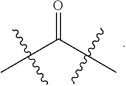 [SMILES: *C(*)=O]

In some variations of formula (25) described in the paragraphs above in which l₁ is 4, Rₐ₁ and R_{b1} are H, Rₐ₂ and R_{b2} are H, Rₐ₃ and R_{b3} are

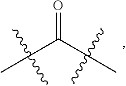 [SMILES: *C(*)=O]

and Rₐ₄ and R_{b4} are v. In some variations of formula (25) described in the paragraphs above in which l₁ is 4, Rₐ₁ and R_{b1} are H, Rₐ₂ and R_{b2} are CH₃, Rₐ₃ and R_{b3} are

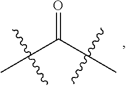 [SMILES: *C(*)=O]

and Rₐ₄ and R_{b4} are

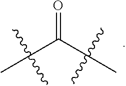 [SMILES: *C(*)=O]

In some variations of formula (25) described in the paragraphs above in which l₁ is 4, Rₐ₁ and R_{b1} are CH₃, Rₐ₂ and R_{b2} are CH₃, Rₐ₃ and R_{b3} are

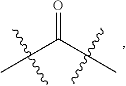 [SMILES: *C(*)=O]

and Rₐ₄ and R_{b4} are

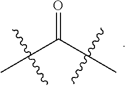 [SMILES: *C(*)=O]

In some variations of formula (25) described in the paragraphs above in which l₁ is 4, Rₐ₁ is H, R_{b1} is CH₃, Rₐ₂ and R_{b2} are CH₃, Rₐ₃ and R_{b3} are

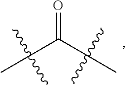 [SMILES: *C(*)=O]

and Rₐ₄ and R_{b4} are

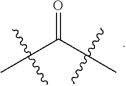 [SMILES: *C(*)=O]

In some variations of formula (25) described in the paragraphs above in which l₁ is 4, Rₐ₁ is H, R_{b1} is CH₃, Rₐ₂ is H, R_{b2} is CH₃, Rₐ₃ and R_{b3} are

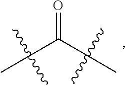 [SMILES: *C(*)=O]

and Rₐ₄ and R_{b4} are

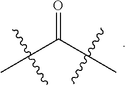 [SMILES: *C(*)=O]

In some variations of formula (25) described in the paragraphs above in which l₁ is 4, Rₐ₁ and R_{b1} are H, Rₐ₂ and R_{b2} are

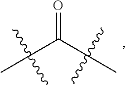 [SMILES: *C(*)=O]

Rₐ₃ and R_{b3} are

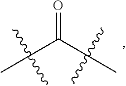 [SMILES: *C(*)=O]

and Rₐ₄ and R_{b4} are

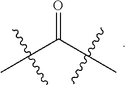 [SMILES: *C(*)=O]

In some variations of formula (25) described in the paragraphs above in which l₁ is 4, Rₐ₁ and R_{b1} are CH₃, Rₐ₂ and R_{b2} are

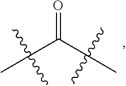 [SMILES: *C(*)=O]

Rₐ₃ and R_{b3} are

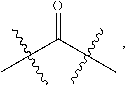 [SMILES: *C(*)=O]

and Rₐ₄ and R_{b4} are

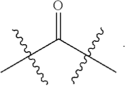 [SMILES: *C(*)=O]

In some variations of formula (25) described in the paragraphs above in which l₁ is 4, Rₐ₁ is H, R_{b1} is CH₃, Rₐ₂ and R_{b2} are

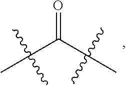 [SMILES: *C(*)=O]

Rₐ₃ and R_{b3} are

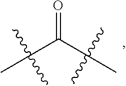 [SMILES: *C(*)=O]

and Rₐ₄ and R_{b4} are

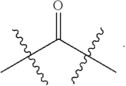 [SMILES: *C(*)=O]

In some variations of formula (25) described in the paragraphs above in which l₁ is 4, Rₐ₁ and R_{b1} are

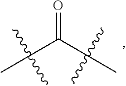 [SMILES: *C(*)=O]

Rₐ₂ and R_{b2} are

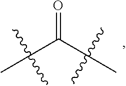 [SMILES: *C(*)=O]

Rₐ₃ and R_{b3} are

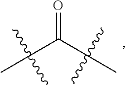 [SMILES: *C(*)=O]

and Rₐ₄ and R_{b4} are

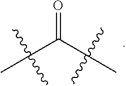 [SMILES: *C(*)=O]

In some variations of formula (25) described in the paragraphs above in which l₁ is 4, Rₐ₁ and R_{b1} are

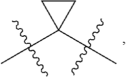 [SMILES: *C1(*)CC1]

Rₐ₂ and R_{b2} are H, Rₐ₃ and R_{b3} are H, and Rₐ₄ and R_{b4} are

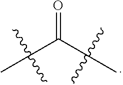 [SMILES: *C(*)=O]

In some variations of formula (25) described in the paragraphs above in which l₁ is 4, Rₐ₁ and R_{b1} are

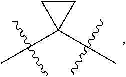 [SMILES: *C1(*)CC1]

Rₐ₂ and R_{b2} are CH₃, Rₐ₃ and R_{b3} are CH₃, and Rₐ₄ and R_{b4} are

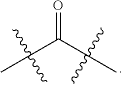 [SMILES: *C(*)=O]

In some variations of formula (25) described in the paragraphs above in which l₁ is 4, Rₐ₁ and R_{b1} are

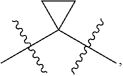 [SMILES: *C1(*)CC1]

Rₐ₂ and R_{b2} are H, Rₐ₃ and R_{b3} are CH₃, and Rₐ₄ and R_{b4} are

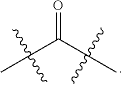 [SMILES: *C(*)=O]

In some variations of formula (25) described in the paragraphs above in which l₁ is 4, Rₐ₁ and R_{b1} are

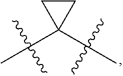 [SMILES: *C1(*)CC1]

Rₐ₂ and R_{b2} are

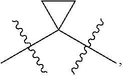 [SMILES: *C1(*)CC1]

Rₐ₃ and R_{b3} are H, and Rₐ₄ and R_{b4} are

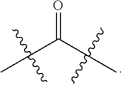 [SMILES: *C(*)=O]

In some variations of formula (25) described in the paragraphs above in which l₁ is 4, Rₐ₁ and R_{b1} are

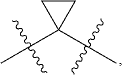 [SMILES: *C1(*)CC1]

Rₐ₂ and R_{b2} are

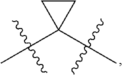 [SMILES: *C1(*)CC1]

Rₐ₃ and R_{b3} are CH₃, and Rₐ₄ and R_{b4} are

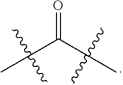 [SMILES: *C(*)=O]

In some variations of formula (25) described in the paragraphs above in which l₁ is 4, Rₐ₁ and R_{b1} are

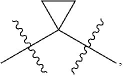 [SMILES: *C1(*)CC1]

Rₐ₂ and R_{b2} are

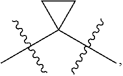 [SMILES: *C1(*)CC1]

Rₐ₃ and R_{b3} are CH₃, and Rₐ₄ and R_{b4} are

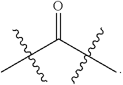 [SMILES: *C(*)=O]

In some variations of formula (25) described in the paragraphs above in which l₁ is 4, Rₐ₁ and R_{b1} are

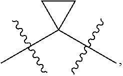 [SMILES: *C1(*)CC1]

Rₐ₂ and R_{b2} are

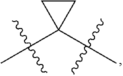 [SMILES: *C1(*)CC1]

Rₐ₃ and R_{b3} are H, and Rₐ₄ and R_{b4} are

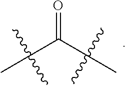 [SMILES: *C(*)=O]

In some variations of formula (25) described in the paragraphs above in which l₁ is 4, Rₐ₁ and R_{b1} are

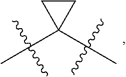 [SMILES: *C1(*)CC1]

Rₐ₂ and R_{b2} are

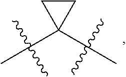 [SMILES: *C1(*)CC1]

Rₐ₃ and R_{b3} are

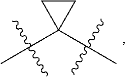 [SMILES: *C1(*)CC1]

and Rₐ₄ and R_{b4} are

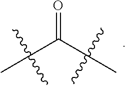 [SMILES: *C(*)=O]

In some variations of formula (25) described in the paragraphs above in which l₁ is 4, Rₐ₁ and R_{b1} are

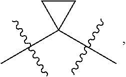 [SMILES: *C1(*)CC1]

Rₐ₂ and R_{b2} are

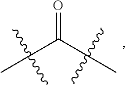 [SMILES: *C(*)=O]

Rₐ₃ and R_{b3} are H, and Rₐ₄ and R_{b4} are

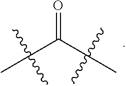 [SMILES: *C(*)=O]

In some variations of formula (25) described in the paragraphs above in which l₁ is 4, Rₐ₁ and R_{b1} are

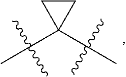 [SMILES: *C1(*)CC1]

Rₐ₂ and R_{b2} are

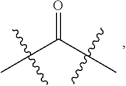 [SMILES: *C(*)=O]

Rₐ₃ and R_{b3} are CH₃, and Rₐ₄ and R_{b4} are

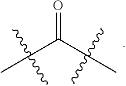 [SMILES: *C(*)=O]

In some variations of formula (25) described in the paragraphs above in which l₁ is 4, Rₐ₁ and R_{b1} are

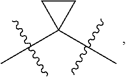 [SMILES: *C1(*)CC1]

Rₐ₂ and R_{b2} are

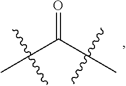 [SMILES: *C(*)=O]

Rₐ₃ and R_{b3} are CH₃, and Rₐ₄ and R_{b4} are

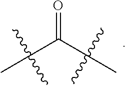 [SMILES: *C(*)=O]

In some variations of formula (25) described in the paragraphs above in which l₁ is 4, Rₐ₁ and R_{b1} are

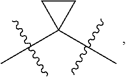 [SMILES: *C1(*)CC1]

Rₐ₂ and R_{b2} are

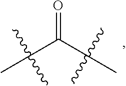 [SMILES: *C(*)=O]

Rₐ₃ and R_{b3} are H, and Rₐ₄ and R_{b4} are

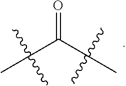 [SMILES: *C(*)=O]

In some variations of formula (25) described in the paragraphs above in which l₁ is 4, Rₐ₁ and R_{b1} are

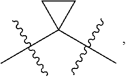 [SMILES: *C1(*)CC1]

Rₐ₂ and R_{b2} are

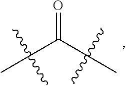 [SMILES: *C(*)=O]

Rₐ₃ and R_{b3} are

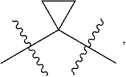 [SMILES: *C1(*)CC1]

and Rₐ₄ and R_{b4} are

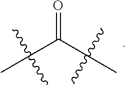 [SMILES: *C(*)=O]

In some variations of formula (25) described in the paragraphs above in which l₁ is 4, Rₐ₁ and R_{b1} are

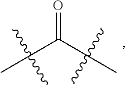 [SMILES: *C(*)=O]

Rₐ₂ and R_{b2} are

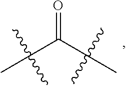 [SMILES: *C(*)=O]

Rₐ₃ and R_{b3} are H, and Rₐ₄ and R_{b4} are

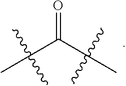 [SMILES: *C(*)=O]

In some variations of formula (25) described in the paragraphs above in which l₁ is 4, Rₐ₁ and R_{b1} are

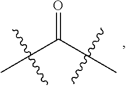 [SMILES: *C(*)=O]

Rₐ₂ and R_{b2} are

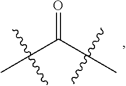 [SMILES: *C(*)=O]

Rₐ₃ and R_{b3} are CH₃, and Rₐ₄ and R_{b4} are

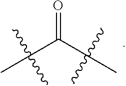 [SMILES: *C(*)=O]

In some variations of formula (25) described in the paragraphs above in which l₁ is 4, Rₐ₁ and R_{b1} are

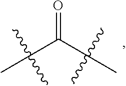 [SMILES: *C(*)=O]

Rₐ₂ and R_{b2} are

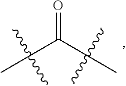 [SMILES: *C(*)=O]

Rₐ₃ and R_{b3} are CH₃, and Rₐ₄ and R_{b4} are

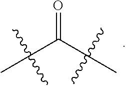 [SMILES: *C(*)=O]

In some variations of formula (25) described in the paragraphs above in which l₁ is 4, Rₐ₁ and R_{b1} are

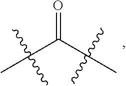 [SMILES: *C(*)=O]

Rₐ₂ and R_{b2} are

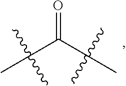 [SMILES: *C(*)=O]

Rₐ₃ and R_{b3} are H, and Rₐ₄ and R_{b4} are

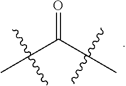 [SMILES: *C(*)=O]

In some variations of formula (25) described in the paragraphs above in which l₁ is 4, Rₐ₁ and R_{b1} are

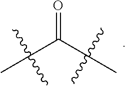 [SMILES: *C(*)=O]

Rₐ₂ and R_{b2} are

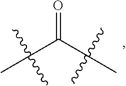 [SMILES: *C(*)=O]

Rₐ₃ and R_{b3} are

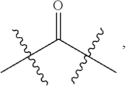 [SMILES: *C(*)=O]

and Rₐ₄ and R_{b4} are

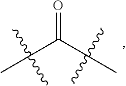 [SMILES: *C(*)=O]

In some variations of formula (25) described in the paragraphs above in which l₁ is 4, Rₐ₁ and R_{b1} are H, Rₐ₂ and R_{b2} are H, and Rₐ₃ and R_{b3} and Rₐ₄ and R_{b4} together are

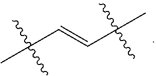 [SMILES: */C=C/*]

In some variations of formula (25) described in the paragraphs above in which l₁ is 4, Rₐ₁ and R_{b1} are H, Rₐ₂ and R_{b2} are CH₃, and Rₐ₃ and R_{b3} and Rₐ₄ and R_{b4} together are

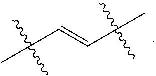 [SMILES: */C=C/*]

In some variations of formula (25) described in the paragraphs above in which l₁ is 4, Rₐ₁ and Rₐ₂ are H, R_{b1} and R_{b2} are CH₃, and Rₐ₃ and R_{b3} and Rₐ₄ and R_{b4} together are

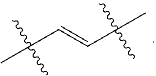 [SMILES: */C=C/*]

In some variations of formula (25) described in the paragraphs above in which l₁ is 4, Rₐ₁ and R_{b1} are H, Rₐ₂ and R_{b2} are

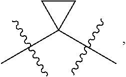 [SMILES: *C1(*)CC1]

and Rₐ₃ and R_{b3} and Rₐ₄ and R_{b4} together are

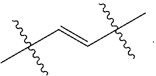 [SMILES: */C=C/*]

In some variations of formula (25) described in the paragraphs above in which l₁ is 4, Rₐ₁ and R_{b1} are

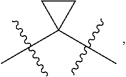 [SMILES: *C1(*)CC1]

Rₐ₂ and R_{b2} are CH₃, and Rₐ₃ and R_{b3} and Rₐ₄ and R_{b4} together are

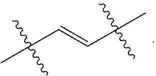 [SMILES: */C=C/*]

In some variations of formula (25) described in the paragraphs above in which l₁ is 4, Rₐ₁ is H, R_{b1} is CH₃, Rₐ₂ and R_{b2} are

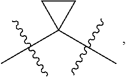 [SMILES: *C1(*)CC1]

and Rₐ₃ and R_{b3} and Rₐ₄ and R_{b4} together are

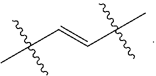 [SMILES: */C=C/*]

In some variations of formula (25) described in the paragraphs above in which l₁ is 4, Rₐ₁ and R_{b1} are H, Rₐ₂ and R_{b2} are

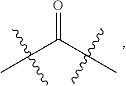 [SMILES: *C(*)=O]

and Rₐ₃ and R_{b3} and Rₐ₄ and R_{b4} together are

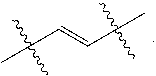 [SMILES: */C=C/*]

In some variations of formula (25) described in the paragraphs above in which l₁ is 4, Rₐ₁ and R_{b1} are

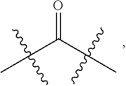 [SMILES: *C(*)=O]

Rₐ₂ and R_{b2} are CH₃, and Rₐ₃ and R_{b3} and Rₐ₄ and R_{b4} together are

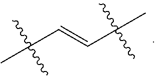 [SMILES: */C=C/*]

In some variations of formula (25) described in the paragraphs above in which l₁ is 4, Rₐ₁ is H, R_{b1} is CH₃, Rₐ₂ and R_{b2} are

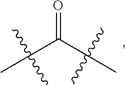 [SMILES: *C(*)=O]

and Rₐ₃ and R_{b3} and Rₐ₄ and R_{b4} together are

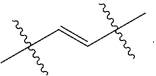 [SMILES: */C=C/*]

In some variations of formula (25) described in the paragraphs above in which l₁ is 4, Rₐ₁ and R_{b1} are

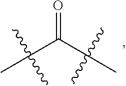 [SMILES: *C(*)=O]

Rₐ₂ and R_{b2} are

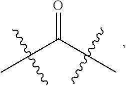 [SMILES: *C(*)=O]

and Rₐ₃ and R_{b3} and Rₐ₄ and R_{b4} together are

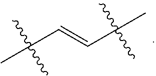 [SMILES: */C=C/*]

In some variations of formula (25) described in the paragraphs above in which l₁ is 4, Rₐ₁ and R_{b1} are

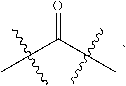 [SMILES: *C(*)=O]

Rₐ₂ and R_{b2} are

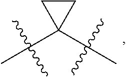 [SMILES: *C1(*)CC1]

and Rₐ₃ and R_{b3} and Rₐ₄ and R_{b4} together are

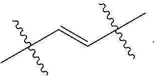 [SMILES: */C=C/*]

In some variations of formula (25) described in the paragraphs above in which l₁ is 4, Rₐ₁ and R_{b1} are

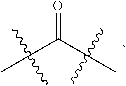 [SMILES: *C(*)=O]

Rₐ₂ and R_{b2} are

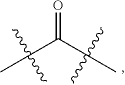 [SMILES: *C(*)=O]

and Rₐ₃ and R_{b3} and Rₐ₄ and R_{b4} together are

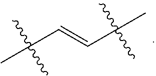 [SMILES: */C=C/*]

In some variations of formula (25) described in the paragraphs above in which l₁ is 4, Rₐ₁ and R_{b1} are

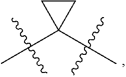 [SMILES: *C1(*)CC1]

Rₐ₂ and R_{b2} are

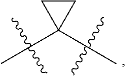 [SMILES: *C1(*)CC1]

and Rₐ₃ and R_{b3} and Rₐ₄ and R_{b4} together are

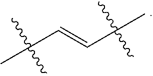 [SMILES: */C=C/*]

In some variations of formula (25) described in the paragraphs above in which l₁ is 4, Rₐ₁ and R_{b1} and Rₐ₂ and R_{b2} together are

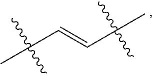 [SMILES: */C=C/*]

and Rₐ₁ and R_{b3} and Rₐ₄ and R_{b4} together are

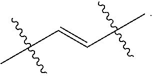 [SMILES: */C=C/*]

In some variations of formula (25) described in the paragraphs above in which l₂ is 1, Rₐ₁ and R_{b1} are both H. In some variations of formula (25) described in the paragraphs above in which l₂ is 1, Rₐ₁ is H and R_{b1} is CH₃. In some variations of formula (25) described in the paragraphs above in which l₂ is 1, Rₐ₁ and R_{b1} are both CH₃. In some variations of formula (25) described in the paragraphs above in which l₂ is 1, Rₐ₁ and R_{b1} are

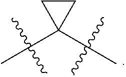 [SMILES: *C1(*)CC1]

In some variations of formula (25) described in the paragraphs above in which l₂ is 1, Rₐ₁ and R_{b1} are

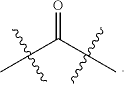 [SMILES: *C(*)=O]

In variations of formula (25) described in the paragraphs above in which l₂ is 2, the two instances of Rₐ and R_{b} are indicated as Rₐ₁ and R_{b1} and Rₐ₂ and R_{b2}, respectively. In some variations of formula (25) described in the paragraphs above in which l₂ is 2, each of Rₐ₁ and R_{b1} and Rₐ₂ and R_{b2} is H. In some variations of formula (25) described in the paragraphs above in which l₂ is 2, each of Rₐ₁ and R_{b1} and Rₐ₂ and R_{b2} is CH₃. In some variations of formula (25) described in the paragraphs above in which l₂ is 2, each of Rₐ₁ and Rₐ₂ is H and each of R_{b1} and R_{b2} is CH₃. In some variations of formula (25) described in the paragraphs above in which l₂ is 2, each of Rₐ₁, Rₐ₂, and R_{b1} is H and R_{b2} is CH₃.

In some variations of formula (25) described in the paragraphs above in which l₂ is 2, each of Rₐ₁ and R_{b1} is H and Rₐ₂ and R_{b2} are

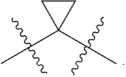 [SMILES: *C1(*)CC1]

In some variations of formula (25) described in the paragraphs above in which l₂ is 2, each of Rₐ₁ and R_{b1} is H and Rₐ₂ and R_{b2} are

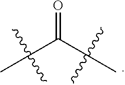 [SMILES: *C(*)=O]

In some variations of formula (25) described in the paragraphs above in which l₂ is 2, each of Rₐ₁ and R_{b1} is CH₃ and Rₐ₂ and R_{b2} are

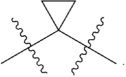 [SMILES: *C1(*)CC1]

In some variations of formula (25) described in the paragraphs above in which l₂ is 2, each of Rₐ₁ and R_{b1} is CH₃ and Rₐ₂ and R_{b2} are

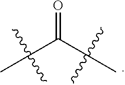 [SMILES: *C(*)=O]

In some variations of formula (25) described in the paragraphs above in which l₂ is 2, Rₐ₁ is H, R_{b1} is CH₃, and Rₐ₂ and R_{b2} are

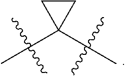 [SMILES: *C1(*)CC1]

In some variations of formula (25) described in the paragraphs above in which l₂ is 2, Rₐ₁ is H, R_{b1} is CH₃, and Rₐ₂ and R_{b2} are

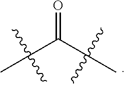 [SMILES: *C(*)=O]

In some variations of formula (25) described in the paragraphs above in which l₂ is 2, Rₐ₁ and R_{b1} and Rₐ₂ and R_{b2} together are

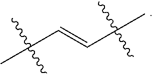 [SMILES: */C=C/*]

In some variations of formula (25) described in the paragraphs above in which l₂ is 2, Rₐ₁ and R_{b1} are

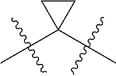 [SMILES: *C1(*)CC1]

Rₐ₂ and R_{b2} are

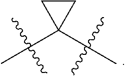 [SMILES: *C1(*)CC1]

In some variations of formula (25) described in the paragraphs above in which l₂ is 2, Rₐ₁ and R_{b1} are

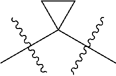 [SMILES: *C1(*)CC1]

Rₐ₂ and R_{b2} are

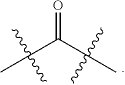 [SMILES: *C(*)=O]

In some variations of formula (25) described in the paragraphs above in which l₂ is 2, Rₐ₁ and R_{b1} are

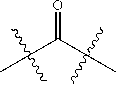 [SMILES: *C(*)=O]

Rₐ₂ and R_{b2} are

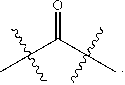 [SMILES: *C(*)=O]

In variations of formula (25) described in the paragraphs above in which l₂ is 3, the three instances of Rₐ and R_{b} are indicated as Rₐ₁ and R_{b1}; Rₐ₂ and R_{b2}; and Rₐ₃ and R_{b3}, respectively. In some variations of formula (25) described in the paragraphs above in which l₂ is 3, In some variations of formula (25) described in the paragraphs above in which l₂ is 3, each of Rₐ₁, R_{b1}, Rₐ₂, R_{b2}, Rₐ₃, and R_{b3} is H. In some variations of formula (25) described in the paragraphs above in which l₂ is 3, each of Rₐ₁, R_{b1}, Rₐ₂, R_{b2}, Rₐ₃, and R_{b3} is CH₃. In some variations of formula (25) described in the paragraphs above in which l₂ is 3, each of Rₐ₁, R_{b1}, Rₐ₂, and R_{b2}, is H and each of Rₐ₃ and R_{b3} is CH₃. In some variations of formula (25) described in the paragraphs above in which l₂ is 3, each of Rₐ₁, R_{b1}, Rₐ₂, and R_{b2}, is CH₃ and each of Rₐ₃ and R_{b3} is H. In some variations of formula (25) described in the paragraphs above in which l₂ is 3, Rₐ₁ is H and each of Rₐ₂, Rₐ₃, R_{b1}, R_{b2}, and R_{b3} is CH₃. In some variations of formula (25) described in the paragraphs above in which l₂ is 3, each of Rₐ₁ and Rₐ₂ is H and each of Rₐ₃, R_{b1}, R_{b2}, an R_{b3} is CH₃. In some variations of formula (25) described in the paragraphs above in which l₂ is 3, each of Rₐ₁, Rₐ₂, and Rₐ₃ is H and each of R_{b1}, R_{b2}, an R_{b3} is CH₃.

In some variations of formula (25) described in the paragraphs above in which l₂ is 3, each of Rₐ₁, R_{b1}, Rₐ₂, and R_{b2}, is H and Rₐ₃ and R_{b3} are

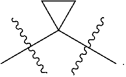 [SMILES: *C1(*)CC1]

In some variations of formula (25) described in the paragraphs above in which l₂ is 3, each of Rₐ₁, R_{b1}, Rₐ₂, and R_{b2}, is CH₃ and Rₐ₃ and R_{b3} are

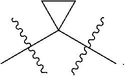 [SMILES: *C1(*)CC1]

In some variations of formula (25) described in the paragraphs above in which l₂ is 3, Rₐ₁ is H and each of Rₐ₂, R_{b1}, and R_{b2} is CH₃, and Rₐ₃ and R_{b3} are

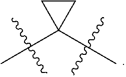 [SMILES: *C1(*)CC1]

In some variations of formula (25) described in the paragraphs above in which l₂ is 3, each of Rₐ₁ and Rₐ₂ is H and each of R_{b1} and R_{b2} is CH₃, and Rₐ₃ and R_{b3} are

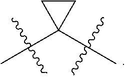 [SMILES: *C1(*)CC1]

In some variations of formula (25) described in the paragraphs above in which l₂ is 3, each of Rₐ₁, R_{b1}, Rₐ₂, and R_{b2}, is H and Rₐ₃ and R_{b3} are

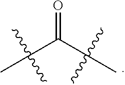 [SMILES: *C(*)=O]

In some variations of formula (25) described in the paragraphs above in which l₂ is 3, each of Rₐ₁, R_{b1}, Rₐ₂, and R_{b2}, is CH₃ and Rₐ₃ and R_{b3} are

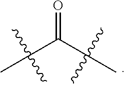 [SMILES: *C(*)=O]

In some variations of formula (25) described in the paragraphs above in which l₂ is 3, Rₐ₁ is H and each of Rₐ₂, R_{b1}, and R_{b2} is CH₃, and Rₐ₃ and R_{b3} are

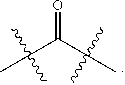 [SMILES: *C(*)=O]

In some variations of formula (25) described in the paragraphs above in which l₂ is 3, each of Rₐ₁ and Rₐ₂ is H and each of R_{b1} and R_{b2} is CH₃, and Rₐ₃ and R_{b3} are

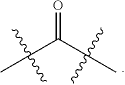 [SMILES: *C(*)=O]

In some variations of formula (25) described in the paragraphs above in which l₂ is 3, Rₐ₁ and R_{b1} are

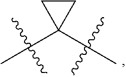 [SMILES: *C1(*)CC1]

Rₐ₂ and R_{b2} are

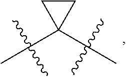 [SMILES: *C1(*)CC1]

and each of Rₐ₃ and R_{b3} is H. In some variations of formula (25) described in the paragraphs above in which l₂ is 3, Rₐ₁ and R_{b1} are

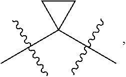 [SMILES: *C1(*)CC1]

Rₐ₂ and R_{b2} are

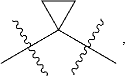 [SMILES: *C1(*)CC1]

and each of Rₐ₃ and R_{b3} is CH₃. In some variations of formula (25) described in the paragraphs above in which l₂ is 3, Rₐ₁ and R_{b1} are

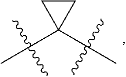 [SMILES: *C1(*)CC1]

Rₐ₂ and R_{b2} are

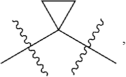 [SMILES: *C1(*)CC1]

Rₐ₃ is H, and R_{b3} is CH₃. In some variations of formula (25) described in the paragraphs above in which l₂ is 3, Rₐ₁ and R_{b1} are

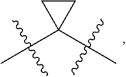 [SMILES: *C1(*)CC1]

Rₐ₂ and R_{b2} are

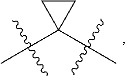 [SMILES: *C1(*)CC1]

and each of Rₐ₃ and R_{b3} is H. In some variations of formula (25) described in the paragraphs above in which l₂ is 3, Rₐ₁ and R_{b1} are

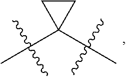 [SMILES: *C1(*)CC1]

Rₐ₂ and R_{b2} are

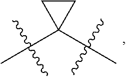 [SMILES: *C1(*)CC1]

and each of Rₐ₃ and R_{b3} is CH₃. In some variations of formula (25) described in the paragraphs above in which l₂ is 3, Rₐ₁ and R_{b1} are

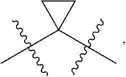 [SMILES: *C1(*)CC1]

Rₐ₂ and R_{b2} are

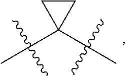 [SMILES: *C1(*)CC1]

Rₐ₃ is H, and R_{b3} is CH₃.

In some variations of formula (25) described in the paragraphs above in which l₂ is 3, Rₐ₁ and R_{b1} are

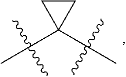 [SMILES: *C1(*)CC1]

Rₐ₂ and R_{b2} are

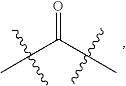 [SMILES: *C(*)=O]

and each of Rₐ₃ and R_{b3} is H. In some variations of formula (25) described in the paragraphs above in which l₂ is 3, Rₐ₁ and R_{b1} are

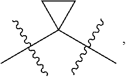 [SMILES: *C1(*)CC1]

Rₐ₂ and R_{b2} are

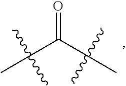 [SMILES: *C(*)=O]

and each of Rₐ₃ and R_{b3} is CH₃. In some variations of formula (25) described in the paragraphs above in which l₂ is 3, Rₐ₁ and R_{b1} are

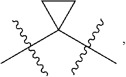 [SMILES: *C1(*)CC1]

Rₐ₂ and R_{b2} are

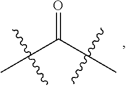 [SMILES: *C(*)=O]

Rₐ₃ is H, and R_{b3} is CH₃. In some variations of formula (25) described in the paragraphs above in which l₂ is 3, Rₐ₁ and R_{b1} are

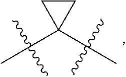 [SMILES: *C1(*)CC1]

Rₐ₂ and R_{b2} are

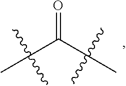 [SMILES: *C(*)=O]

and each of Rₐ₃ and R_{b3} is H. In some variations of formula (25) described in the paragraphs above in which l₂ is 3, Rₐ₁ and R_{b1} are

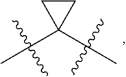 [SMILES: *C1(*)CC1]

Rₐ₂ and R_{b2} are

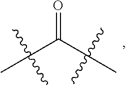 [SMILES: *C(*)=O]

and each of Rₐ₃ and R_{b3} is CH₃. In some variations of formula (25) described in the paragraphs above in which l₂ is 3, Rₐ₁ and R_{b1} are

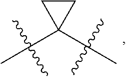 [SMILES: *C1(*)CC1]

Rₐ₂ and R_{b2} are

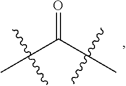 [SMILES: *C(*)=O]

Rₐ₃ is H, and R_{b3} is CH₃.

In some variations of formula (25) described in the paragraphs above in which l₂ is 3, Rₐ₁ and R_{b1} are

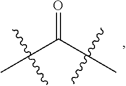 [SMILES: *C(*)=O]

Rₐ₂ and R_{b2} are

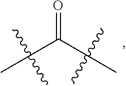 [SMILES: *C(*)=O]

and each of Rₐ₃ and R_{b3} is H. In some variations of formula (25) described in the paragraphs above in which l₂ is 3, Rₐ₁ and R_{b1} are

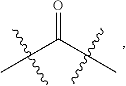 [SMILES: *C(*)=O]

Rₐ₂ and R_{b2} are

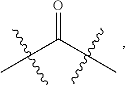 [SMILES: *C(*)=O]

and each of Rₐ₃ and R_{b3} is CH₃. In some variations of formula (25) described in the paragraphs above in which l₂ is 3, Rₐ₁ and R_{b1} are

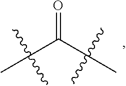 [SMILES: *C(*)=O]

Rₐ₂ and R_{b2} are

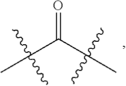 [SMILES: *C(*)=O]

Rₐ₃ is H, and R_{b3} is CH₃. In some variations of formula (25) described in the paragraphs above in which l₂ is 3, Rₐ₁ and R_{b1} are

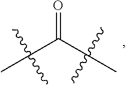 [SMILES: *C(*)=O]

Rₐ₂ and R_{b2} are

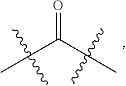 [SMILES: *C(*)=O]

and each of Rₐ₃ and R_{b3} is H. In some variations of formula (25) described in the paragraphs above in which l₂ is 3, Rₐ₁ and R_{b1} are

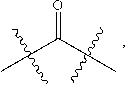 [SMILES: *C(*)=O]

Rₐ₂ and R_{b2} are

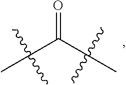 [SMILES: *C(*)=O]

and each of Rₐ₃ and R_{b3} is CH₃. In some variations of formula (25) described in the paragraphs above in which l₂ is 3, Rₐ₁ and R_{b1} are

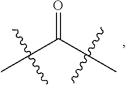 [SMILES: *C(*)=O]

Rₐ₂ and R_{b2} are

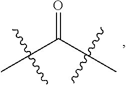 [SMILES: *C(*)=O]

Rₐ₃ is H, and R_{b3} is CH₃.

In some variations of formula (25) described in the paragraphs above in which l₂ is 3, Rₐ₁ and R_{b1} and Rₐ₂ and R_{b2} together are

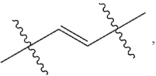 [SMILES: */C=C/*]

and each of Rₐ₃ and R_{b3} is H. In some variations of formula (25) described in the paragraphs above in which l₂ is 3, Rₐ₁ and R_{b1} and Rₐ₂ and R_{b2} together are

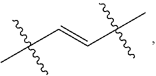 [SMILES: */C=C/*]

and each of Rₐ₃ and R_{b3} is CH₃. In some variations of formula (25) described in the paragraphs above in which l₂ is 3, Rₐ₁ and R_{b1} and Rₐ₂ and R_{b2} together are

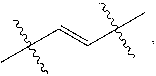 [SMILES: */C=C/*]

Rₐ₃ is H, and R_{b3} is CH₃. In some variations of formula (25) described in the paragraphs above in which l₂ is 3, Rₐ₁ and R_{b1} and Rₐ₂ and R_{b2} together are

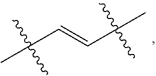 [SMILES: */C=C/*]

and Rₐ₃ and R_{b3} are

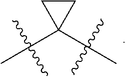 [SMILES: *C1(*)CC1]

In some variations of formula (25) described in the paragraphs above in which l₂ is 3, Rₐ₁ and R_{b1} and Rₐ₂ and R_{b2} together are

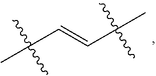 [SMILES: */C=C/*]

and Rₐ₃ and R_{b3} are

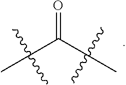 [SMILES: *C(*)=O]

In variations of formula (25) described in the paragraphs above in which l₂ is 4, the three instances of Rₐ and R_{b} are indicated as Rₐ₁ and R_{b1}; Rₐ₂ and R_{b2}; Rₐ₃ and R_{b3}; and Rₐ₄ and R_{b4}, respectively. In some variations of formula (25) described in the paragraphs above in which l₂ is 4, In some variations of formula (25) described in the paragraphs above in which l₂ is 4, each of Rₐ₁, R_{b1}, Rₐ₂, R_{b2}, Rₐ₃, R_{b3}, Rₐ₄, and R_{b4} is H. In some variations of formula (25) described in the paragraphs above in which l₂ is 4, each of Rₐ₁, R_{b1}, Rₐ₂, R_{b2}, Rₐ₃, R_{b3}, Rₐ₄, and R_{b4} is CH₃. In some variations of formula (25) described in the paragraphs above in which l₂ is 4, each of Rₐ₁, R_{b1}, Rₐ₂, R_{b2}, Rₐ₃, and R_{b3} is H and each of Rₐ₄ and R_{b4} is CH₃. In some variations of formula (25) described in the paragraphs above in which l₂ is 4, each of Rₐ₁, R_{b1}, Rₐ₂, and R_{b2} is H and each of Rₐ₃, R_{b3}, Rₐ₄, and R_{b4} is CH₃. In some variations of formula (25) described in the paragraphs above in which l₂ is 4, each of Rₐ₁, R_{b1}, Rₐ₂, R_{b2}, Rₐ₃, and R_{b3} is CH₃ and each of Rₐ₄ and R_{b4} is H.

In some variations of formula (25) described in the paragraphs above in which l₂ is 4, each of Rₐ₁, Rₐ₂, and Rₐ₃ is H and each of R_{b1}, R_{b2}, and R_{b3} is CH₃. In some variations of formula (25) described in the paragraphs above in which l₂ is 4, Rₐ₁ is H, R_{b1} is CH₃, and each of Rₐ₂, R_{b2}, Rₐ₃, and R_{b3} is CH₃. In some variations of formula (25) described in the paragraphs above in which l₂ is 4, Rₐ₁ is H, R_{b1} is CH₃, and each of Rₐ₂, R_{b2}, Rₐ₃, d R_{b3} and is H. In some variations of formula (25) described in the paragraphs above in which l₂ is 4, each of Rₐ₁ and Rₐ₂ is H, each of R_{b1} and R_{b2} is CH₃, and each of Rₐ₃ and R_{b3} is H. In some variations of formula (25) described in the paragraphs above in which l₂ is 4, each of Rₐ₁ and Rₐ₂ is H, each of R_{b1} and R_{b2} is CH₃, and each of Rₐ₃ and R_{b3} is CH₃.

In some variations of formula (25) described in the paragraphs above in which l₂ is 4, Rₐ₁ and R_{b1} are H, Rₐ₂ and R_{b2} are H, Rₐ₃ and R_{b3} are H, and Rₐ₄ and R_{b4} are

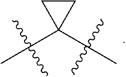 [SMILES: *C1(*)CC1]

In some variations of formula (25) described in the paragraphs above in which l₂ is 4, Rₐ₁ and R_{b1} are H, Rₐ₂ and R_{b2} are H, Rₐ₃ and R_{b3} are CH₃, and Rₐ₄ and R_{b4} are

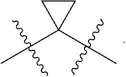 [SMILES: *C1(*)CC1]

In some variations of formula (25) described in the paragraphs above in which l₂ is 4, Rₐ₁ and R_{b1} are H, Rₐ₂ and R_{b2} are CH₃, Rₐ₃ and R_{b3} are CH₃, and Rₐ₄ and R_{b4} are

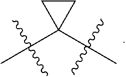 [SMILES: *C1(*)CC1]

In some variations of formula (25) described in the paragraphs above in which l₂ is 4, Rₐ₁ and R_{b1} are CH₃, Rₐ₂ and R_{b2} are CH₃, Rₐ₃ and R_{b3} are CH₃, and Rₐ₄ and R_{b4} are

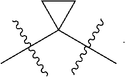 [SMILES: *C1(*)CC1]

In some variations of formula (25) described in the paragraphs above in which l₂ is 4, Rₐ₁ and R_{b1} are H, Rₐ₂ and R_{b2} are H, Rₐ₃ and R_{b3} are

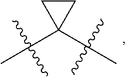 [SMILES: *C1(*)CC1]

and Rₐ₄ and R_{b4} are

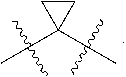 [SMILES: *C1(*)CC1]

In some variations of formula (25) described in the paragraphs above in which l₂ is 4, Rₐ₁ and R_{b1} are H, Rₐ₂ and R_{b2} are CH₃, Rₐ₃ and R_{b3} are

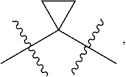 [SMILES: *C1(*)CC1]

and Rₐ₄ and R_{b4} are

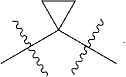 [SMILES: *C1(*)CC1]

In some variations of formula (25) described in the paragraphs above in which l₂ is 4, Rₐ₁ and R_{b1} are CH₃, Rₐ₂ and R_{b2} are CH₃, Rₐ₃ and R_{b3} are

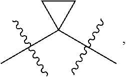 [SMILES: *C1(*)CC1]

and Rₐ₄ and R_{b4} are

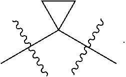 [SMILES: *C1(*)CC1]

In some variations of formula (25) described in the paragraphs above in which l₂ is 4, Rₐ₁ is H, R_{b1} is CH₃, Rₐ₂ and R_{b2} are CH₃, Rₐ₃ and R_{b3} are

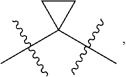 [SMILES: *C1(*)CC1]

and Rₐ₄ and R_{b4} are

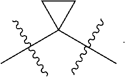 [SMILES: *C1(*)CC1]

In some variations of formula (25) described in the paragraphs above in which l₂ is 4, Rₐ₁ is H, R_{b1} is CH₃, Rₐ₂ is H, R_{b2} CH₃, Rₐ₃ and R_{b3} are

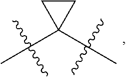 [SMILES: *C1(*)CC1]

and Rₐ₄ and R_{b4} are

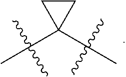 [SMILES: *C1(*)CC1]

In some variations of formula (25) described in the paragraphs above in which l₂ is 4, Rₐ₁ and R_{b1} are H, Rₐ₂ and R_{b2} are

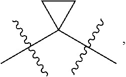 [SMILES: *C1(*)CC1]

Rₐ₃ and R_{b3} are

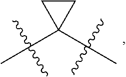 [SMILES: *C1(*)CC1]

and Rₐ₄ and R_{b4} are

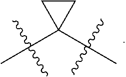 [SMILES: *C1(*)CC1]

In some variations of formula (25) described in the paragraphs above in which l₂ is 4, Rₐ₁ and R_{b1} are CH₃, Rₐ₂ and R_{b2} are

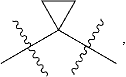 [SMILES: *C1(*)CC1]

Rₐ₃ and R_{b3} are

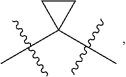 [SMILES: *C1(*)CC1]

and Rₐ₄ and R_{b4} are

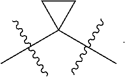 [SMILES: *C1(*)CC1]

In some variations of formula (25) described in the paragraphs above in which l₂ is 4, Rₐ₁ is H, R_{b1} is CH₃, Rₐ₂ and R_{b2} are

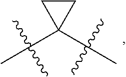 [SMILES: *C1(*)CC1]

Rₐ₃ and R_{b3} are

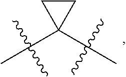 [SMILES: *C1(*)CC1]

and Rₐ₄ and R_{b4} are

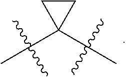 [SMILES: *C1(*)CC1]

In some variations of formula (25) described in the paragraphs above in which l₂ is 4, Rₐ₁ and R_{b1} are

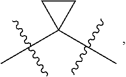 [SMILES: *C1(*)CC1]

Rₐ₂ and R_{b2} are

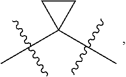 [SMILES: *C1(*)CC1]

Rₐ₃ and R_{b3} are

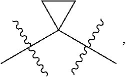 [SMILES: *C1(*)CC1]

and Rₐ₄ and R_{b4} are

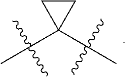 [SMILES: *C1(*)CC1]

In some variations of formula (25) described in the paragraphs above in which l₂ is 4, Rₐ₁ and R_{b1} are H, Rₐ₂ and R_{b2} are H, Rₐ₃ and R_{b3} are H, and Rₐ₄ and R_{b4} are

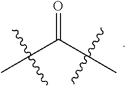 [SMILES: *C(*)=O]

In some variations of formula (25) described in the paragraphs above in which l₂ is 4, Rₐ₁ and R_{b1} are H, Rₐ₂ and R_{b2} are H, Rₐ₃ and R_{b3} are CH₃, and Rₐ₄ and R_{b4} are

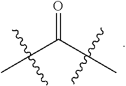 [SMILES: *C(*)=O]

In some variations of formula (25) described in the paragraphs above in which l₂ is 4, Rₐ₁ and R_{b1} are H, Rₐ₂ and R_{b2} are CH₃, Rₐ₃ and R_{b3} are CH₃, and Rₐ₄ and R_{b4} are

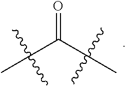 [SMILES: *C(*)=O]

In some variations of formula (25) described in the paragraphs above in which l₂ is 4, Rₐ₁ and R_{b1} are CH₃, Rₐ₂ and R_{b2} are CH₃, Rₐ₃ and R_{b3} are CH₃, and Rₐ₄ and R_{b4} are

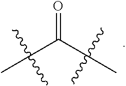 [SMILES: *C(*)=O]

In some variations of formula (25) described in the paragraphs above in which l₂ is 4, Rₐ₁ and R_{b1} are H, Rₐ₂ and R_{b2} are H, Rₐ₃ and R_{b3} are

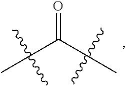 [SMILES: *C(*)=O]

and Rₐ₄ and R_{b4} are v. In some variations of formula (25) described in the paragraphs above in which l₂ is 4, Rₐ₁ and R_{b1} are H, Rₐ₂ and R_{b2} are CH₃, Rₐ₃ and R_{b3} are

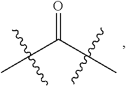 [SMILES: *C(*)=O]

and Rₐ₄ and R_{b4} are

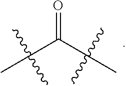 [SMILES: *C(*)=O]

In some variations of formula (25) described in the paragraphs above in which l₂ is 4, Rₐ₁ and R_{b1} are CH₃, Rₐ₂ and R_{b2} are CH₃, Rₐ₃ and R_{b3} are

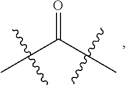 [SMILES: *C(*)=O]

and Rₐ₄ and R_{b4} are

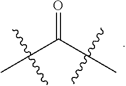 [SMILES: *C(*)=O]

In some variations of formula (25) described in the paragraphs above in which l₂ is 4, Rₐ₁ is H, R_{b1} is CH₃, Rₐ₂ and R_{b2} are CH₃, Rₐ₃ and R_{b3} are

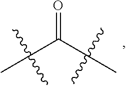 [SMILES: *C(*)=O]

and Rₐ₄ and R_{b4} are

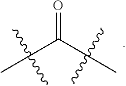 [SMILES: *C(*)=O]

In some variations of formula (25) described in the paragraphs above in which l₂ is 4, Rₐ₁ is H, R_{b1} is CH₃, Rₐ₂ is H, R_{b2} is CH₃, Rₐ₃ and R_{b3} are

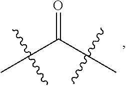 [SMILES: *C(*)=O]

and Rₐ₄ and R_{b4} are

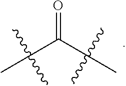 [SMILES: *C(*)=O]

In some variations of formula (25) described in the paragraphs above in which l₂ is 4, Rₐ₁ and R_{b1} are H, Rₐ₂ and R_{b2} are

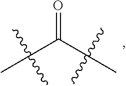 [SMILES: *C(*)=O]

Rₐ₃ and R_{b3} are

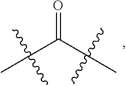 [SMILES: *C(*)=O]

and Rₐ₄ and R_{b4} are

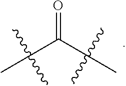 [SMILES: *C(*)=O]

In some variations of formula (25) described in the paragraphs above in which l₂ is 4, Rₐ₁ and R_{b1} are CH₃, Rₐ₂ and R_{b2} are

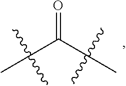 [SMILES: *C(*)=O]

Rₐ₃ and R_{b3} are

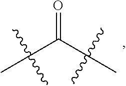 [SMILES: *C(*)=O]

and Rₐ₄ and R_{b4} are

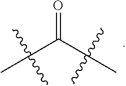 [SMILES: *C(*)=O]

In some variations of formula (25) described in the paragraphs above in which l₂ is 4, Rₐ₁ is H, R_{b1} is CH₃, Rₐ₂ and R_{b2} are

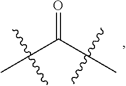 [SMILES: *C(*)=O]

Rₐ₃ and R_{b3} are

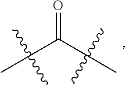 [SMILES: *C(*)=O]

and Rₐ₄ and R_{b4} are

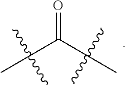 [SMILES: *C(*)=O]

In some variations of formula (25) described in the paragraphs above in which l₂ is 4, Rₐ₁ and R_{b1} are

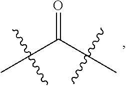 [SMILES: *C(*)=O]

Rₐ₂ and R_{b2} are

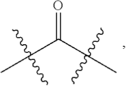 [SMILES: *C(*)=O]

Rₐ₃ and R_{b3} are

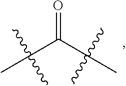 [SMILES: *C(*)=O]

and Rₐ₄ and R_{b4} are

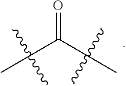 [SMILES: *C(*)=O]

In some variations of formula (25) described in the paragraphs above in which l₂ is 4, Rₐ₁ and R_{b1} are

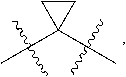 [SMILES: *C1(*)CC1]

Rₐ₂ and R_{b2} are H, Rₐ₃ and R_{b3} are H, and Rₐ₄ and R_{b4} are

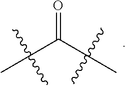 [SMILES: *C(*)=O]

In some variations of formula (25) described in the paragraphs above in which l₂ is 4, Rₐ₁ and R_{b1} are

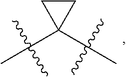 [SMILES: *C1(*)CC1]

Rₐ₂ and R_{b2} are CH₃, Rₐ₃ and R_{b3} are CH₃, and Rₐ₄ and R_{b4} are

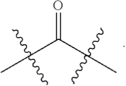 [SMILES: *C(*)=O]

In some variations of formula (25) described in the paragraphs above in which l₂ is 4, Rₐ₁ and R_{b1} are

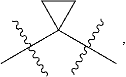 [SMILES: *C1(*)CC1]

Rₐ₂ and R_{b2} are H, Rₐ₃ and R_{b3} are CH₃, and Rₐ₄ and R_{b4} are

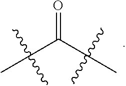 [SMILES: *C(*)=O]

In some variations of formula (25) described in the paragraphs above in which l₂ is 4, Rₐ₁ and R_{b1} are

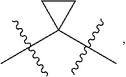 [SMILES: *C1(*)CC1]

Rₐ₂ and R_{b2} are

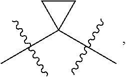 [SMILES: *C1(*)CC1]

Rₐ₃ and R_{b3} are H, and Rₐ₄ and R_{b4} are

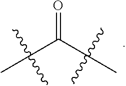 [SMILES: *C(*)=O]

In some variations of formula (25) described in the paragraphs above in which l₂ is 4, Rₐ₁ and R_{b1} are

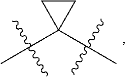 [SMILES: *C1(*)CC1]

Rₐ₂ and R_{b2} are

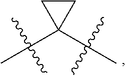 [SMILES: *C1(*)CC1]

Rₐ₃ and R_{b3} are CH₃, and Rₐ₄ and R_{b4} are

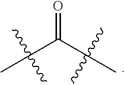 [SMILES: *C(*)=O]

In some variations of formula (25) described in the paragraphs above in which l₂ is 4, Rₐ₁ and R_{b1} are

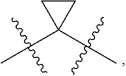 [SMILES: *C1(*)CC1]

Rₐ₂ and R_{b2} are

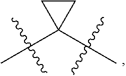 [SMILES: *C1(*)CC1]

Rₐ₃ and R_{b3} are CH₃, and Rₐ₄ and R_{b4} are

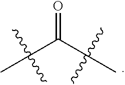 [SMILES: *C(*)=O]

In some variations of formula (25) described in the paragraphs above in which l₂ is 4, Rₐ₁ and R_{b1} are

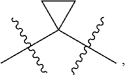 [SMILES: *C1(*)CC1]

Rₐ₂ and R_{b2} are

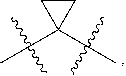 [SMILES: *C1(*)CC1]

Rₐ₃ and R_{b3} are H, and Rₐ₄ and R_{b4} are

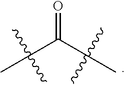 [SMILES: *C(*)=O]

In some variations of formula (25) described in the paragraphs above in which l₂ is 4, Rₐ₁ and R_{b1} are

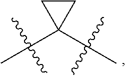 [SMILES: *C1(*)CC1]

Rₐ₂ and R_{b2} are

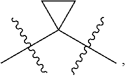 [SMILES: *C1(*)CC1]

Rₐ₃ and R_{b3} are

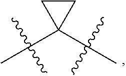 [SMILES: *C1(*)CC1]

and Rₐ₄ and R_{b4} are

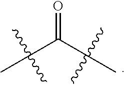 [SMILES: *C(*)=O]

In some variations of formula (25) described in the paragraphs above in which l₂ is 4, Rₐ₁ and R_{b1} are

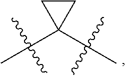 [SMILES: *C1(*)CC1]

Rₐ₂ and R_{b2} are

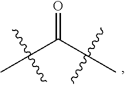 [SMILES: *C(*)=O]

Rₐ₃ and R_{b3} are H, and Rₐ₄ and R_{b4} are

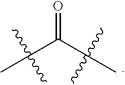 [SMILES: *C(*)=O]

In some variations of formula (25) described in the paragraphs above in which l₂ is 4, Rₐ₁ and R_{b1} are

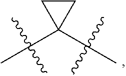 [SMILES: *C1(*)CC1]

Rₐ₂ and R_{b2} are

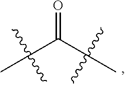 [SMILES: *C(*)=O]

Rₐ₃ and R_{b3} are CH₃, and Rₐ₄ and R_{b4} are

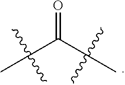 [SMILES: *C(*)=O]

In some variations of formula (25) described in the paragraphs above in which l₂ is 4, Rₐ₁ and R_{b1} are

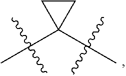 [SMILES: *C1(*)CC1]

Rₐ₂ and R_{b2} are

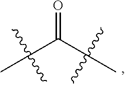 [SMILES: *C(*)=O]

Rₐ₃ and R_{b3} are CH₃, and Rₐ₄ and R_{b4} are

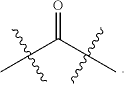 [SMILES: *C(*)=O]

In some variations of formula (25) described in the paragraphs above in which l₂ is 4, Rₐ₁ and R_{b1} are

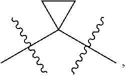 [SMILES: *C1(*)CC1]

Rₐ₂ and R_{b2} are

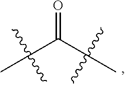 [SMILES: *C(*)=O]

Rₐ₃ and R_{b3} are H, and Rₐ₄ and R_{b4} are

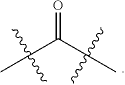 [SMILES: *C(*)=O]

In some variations of formula (25) described in the paragraphs above in which l₂ is 4, Rₐ₁ and R_{b1} are

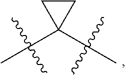 [SMILES: *C1(*)CC1]

Rₐ₂ and R_{b2} are

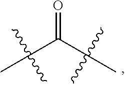 [SMILES: *C(*)=O]

Rₐ₃ and R_{b3} are

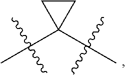 [SMILES: *C1(*)CC1]

and Rₐ₄ and R_{b4} are

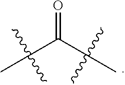 [SMILES: *C(*)=O]

In some variations of formula (25) described in the paragraphs above in which l₂ is 4, Rₐ₁ and R_{b1} are

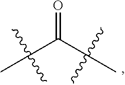 [SMILES: *C(*)=O]

Rₐ₂ and R_{b2} are

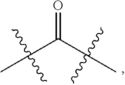 [SMILES: *C(*)=O]

Rₐ₃ and R_{b3} are H, and Rₐ₄ and R_{b4} are

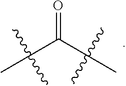 [SMILES: *C(*)=O]

In some variations of formula (25) described in the paragraphs above in which l₂ is 4, Rₐ₁ and R_{b1} are

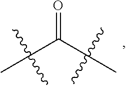 [SMILES: *C(*)=O]

Rₐ₂ and R_{b2} are

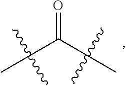 [SMILES: *C(*)=O]

Rₐ₃ and R_{b3} are CH₃, and Rₐ₄ and R_{b4} are

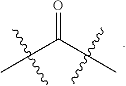 [SMILES: *C(*)=O]

In some variations of formula (25) described in the paragraphs above in which l₂ is 4, Rₐ₁ and R_{b1} are

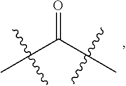 [SMILES: *C(*)=O]

Rₐ₂ and R_{b2} are

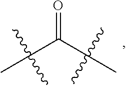 [SMILES: *C(*)=O]

Rₐ₃ and R_{b3} are CH₃, and Rₐ₄ and R_{b4} are

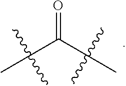 [SMILES: *C(*)=O]

In some variations of formula (25) described in the paragraphs above in which l₂ is 4, Rₐ₁ and R_{b1} are

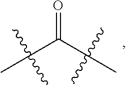 [SMILES: *C(*)=O]

Rₐ₂ and R_{b2} are

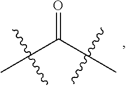 [SMILES: *C(*)=O]

Rₐ₃ and R_{b3} are H, and Rₐ₄ and R_{b4} are

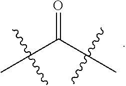 [SMILES: *C(*)=O]

In some variations of formula (25) described in the paragraphs above in which l₂ is 4, Rₐ₁ and R_{b1} are

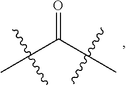 [SMILES: *C(*)=O]

Rₐ₂ and R_{b2} are

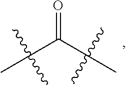 [SMILES: *C(*)=O]

Rₐ₃ and R_{b3} are

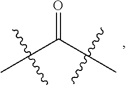 [SMILES: *C(*)=O]

and Rₐ₄ and R_{b4} are

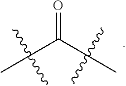 [SMILES: *C(*)=O]

In some variations of formula (25) described in the paragraphs above in which l₂ is 4, Rₐ₁ and R_{b1} are H, Rₐ₂ and R_{b2} are H, and Rₐ₃ and R_{b3} and Rₐ₄ and R_{b4} together are

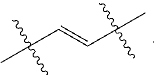 [SMILES: */C=C/*]

In some variations of formula (25) described in the paragraphs above in which l₂ is 4, Rₐ₁ and R_{b1} are H, Rₐ₂ and R_{b2} are CH₃, and Rₐ₃ and R_{b3} and Rₐ₄ and R_{b4} together are

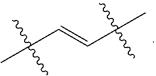 [SMILES: */C=C/*]

In some variations of formula (25) described in the paragraphs above in which l₂ is 4, Rₐ₁ and Rₐ₂ are H, R_{b1} and R_{b2} are CH₃, and Rₐ₃ and R_{b3} and Rₐ₄ and R_{b4} together are

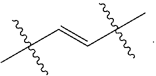 [SMILES: */C=C/*]

In some variations of formula (25) described in the paragraphs above in which l₂ is 4, Rₐ₁ and R_{b1} are H, Rₐ₂ and R_{b2} are

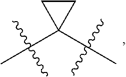 [SMILES: *C1(*)CC1]

and Rₐ₃ and R_{b3} and Rₐ₄ and R_{b4} together are

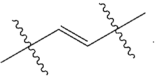 [SMILES: */C=C/*]

In some variations of formula (25) described in the paragraphs above in which l₂ is 4, Rₐ₁ and R_{b1} are

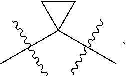 [SMILES: *C1(*)CC1]

Rₐ₂ and R_{b2} are CH₃, and Rₐ₃ and R_{b3} and Rₐ₄ and R_{b4} together are

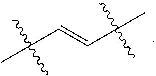 [SMILES: */C=C/*]

In some variations of formula (25) described in the paragraphs above in which l₂ is 4, Rₐ₁ is H, R_{b1} is CH₃, Rₐ₂ and R_{b2} are

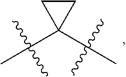 [SMILES: *C1(*)CC1]

and Rₐ₃ and R_{b3} and Rₐ₄ and R_{b4} together are

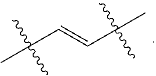 [SMILES: */C=C/*]

In some variations of formula (25) described in the paragraphs above in which l₂ is 4, Rₐ₁ and R_{b1} are H, Rₐ₂ and R_{b2} are

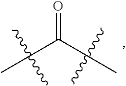 [SMILES: *C(*)=O]

and Rₐ₃ and R_{b3} and Rₐ₄ and R_{b4} together are

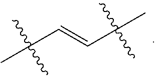 [SMILES: */C=C/*]

In some variations of formula (25) described in the paragraphs above in which l₂ is 4, Rₐ₁ and R_{b1} are

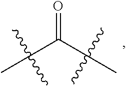 [SMILES: *C(*)=O]

Rₐ₂ and R_{b2} are CH₃, and Rₐ₃ and R_{b3} and Rₐ₄ and R_{b4} together are

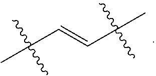 [SMILES: */C=C/*]

In some variations of formula (25) described in the paragraphs above in which l₂ is 4, Rₐ₁ is H, R_{b1} is CH₃, Rₐ₂ and R_{b2} are

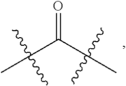 [SMILES: *C(*)=O]

and Rₐ₃ and R_{b3} and Rₐ₄ and R_{b4} together are

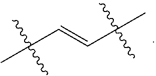 [SMILES: */C=C/*]

In some variations of formula (25) described in the paragraphs above in which l₂ is 4, Rₐ₁ and R_{b1} are

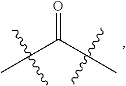 [SMILES: *C(*)=O]

Rₐ₂ and R_{b2} are

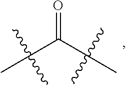 [SMILES: *C(*)=O]

and Rₐ₃ and R_{b3} and Rₐ₄ and R_{b4} together are

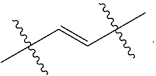 [SMILES: */C=C/*]

In some variations of formula (25) described in the paragraphs above in which l₂ is 4, Rₐ₁ and R_{b1} are

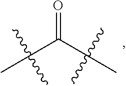 [SMILES: *C(*)=O]

Rₐ₂ and R_{b2} are

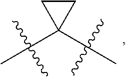 [SMILES: *C1(*)CC1]

and Rₐ₃ and R_{b3} and Rₐ₄ and R_{b4} together are

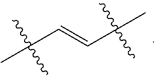 [SMILES: */C=C/*]

In some variations of formula (25) described in the paragraphs above in which l₂ is 4, Rₐ₁ and R_{b1} are

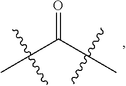 [SMILES: *C(*)=O]

Rₐ₂ and R_{b2} are

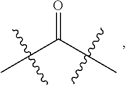 [SMILES: *C(*)=O]

and Rₐ₃ and R_{b3} and Rₐ₄ and R_{b4} together are

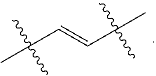 [SMILES: */C=C/*]

In some variations of formula (25) described in the paragraphs above in which l₂ is 4, Rₐ₁ and R_{b1} are

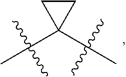 [SMILES: *C1(*)CC1]

Rₐ₂ and R_{b2} are

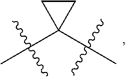 [SMILES: *C1(*)CC1]

and Rₐ₃ and R_{b3} and Rₐ₄ and R_{b4} together are

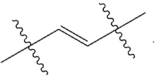 [SMILES: */C=C/*]

In some variations of formula (25) described in the paragraphs above in which l₂ is 4, Rₐ₁ and R_{b1} and Rₐ₂ and R_{b2} together are

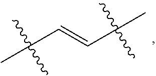 [SMILES: */C=C/*]

and Rₐ₁ and R_{b3} and Rₐ₄ and R_{b4} together are

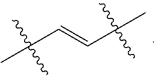 [SMILES: */C=C/*]

In some variations of formula (25) described in the paragraphs above in which o₁ is 1, Rₐ₁ and R_{b1} are both H. In some variations of formula (25) described in the paragraphs above in which o₁ is 1, Rₐ₁ is H and R_{b1} is CH₃. In some variations of formula (25) described in the paragraphs above in which o₁ is 1, Rₐ₁ and R_{b1} are both CH₃. In some variations of formula (25) described in the paragraphs above in which o₁ is 1, Rₐ₁ and R_{b1} are

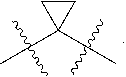 [SMILES: *C1(*)CC1]

In some variations of formula (25) described in the paragraphs above in which o₁ is 1, Rₐ₁ and R_{b1} are

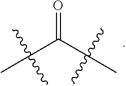 [SMILES: *C(*)=O]

In variations of formula (25) described in the paragraphs above in which o₁ is 2, the two instances of Rₐ and R_{b} are indicated as Rₐ₁ and R_{b1} and Rₐ₂ and R_{b2}, respectively. In some variations of formula (25) described in the paragraphs above in which o₁ is 2, each of Rₐ₁ and R_{b1} and Rₐ₂ and R_{b2} is H. In some variations of formula (25) described in the paragraphs above in which o₁ is 2, each of Rₐ₁ and R_{b1} and Rₐ₂ and R_{b2} is CH₃. In some variations of formula (25) described in the paragraphs above in which o₁ is 2, each of Rₐ₁ and Rₐ₂ is H and each of R_{b1} and R_{b2} is CH₃. In some variations of formula (25) described in the paragraphs above in which o₁ is 2, each of Rₐ₁, Rₐ₂, and R_{b1} is H and R_{b2} is CH₃.

In some variations of formula (25) described in the paragraphs above in which o₁ is 2, each of Rₐ₁ and R_{b1} is H and Rₐ₂ and R_{b2} are

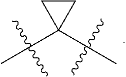 [SMILES: *C1(*)CC1]

In some variations of formula (25) described in the paragraphs above in which o₁ is 2, each of Rₐ₁ and R_{b1} is H and Rₐ₂ and R_{b2} are

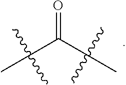 [SMILES: *C(*)=O]

In some variations of formula (25) described in the paragraphs above in which o₁ is 2, each of Rₐ₁ and R_{b1} is CH₃ and Rₐ₂ and R_{b2} are

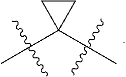 [SMILES: *C1(*)CC1]

In some variations of formula (25) described in the paragraphs above in which o₁ is 2, each of Rₐ₁ and R_{b1} is CH₃ and Rₐ₂ and R_{b2} are

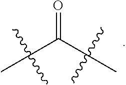 [SMILES: *C(*)=O]

In some variations of formula (25) described in the paragraphs above in which o₁ is 2, Rₐ₁ is H, R_{b1} is CH₃, and Rₐ₂ and R_{b2} are

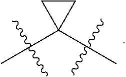 [SMILES: *C1(*)CC1]

In some variations of formula (25) described in the paragraphs above in which o₁ is 2, Rₐ₁ is H, R_{b1} is CH₃, and Rₐ₂ and R_{b2} are

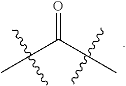 [SMILES: *C(*)=O]

In some variations of formula (25) described in the paragraphs above in which o₁ is 2, Rₐ₁ and R_{b1} and Rₐ₂ and R_{b2} together are

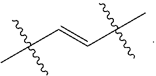 [SMILES: */C=C/*]

In some variations of formula (25) described in the paragraphs above in which o₁ is 2, Rₐ₁ and R_{b1} are

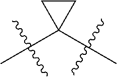 [SMILES: *C1(*)CC1]

Rₐ₂ and R_{b2} are

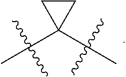 [SMILES: *C1(*)CC1]

In some variations of formula (25) described in the paragraphs above in which o₁ is 2, Rₐ₁ and R_{b1} are

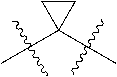 [SMILES: *C1(*)CC1]

Rₐ₂ and R_{b2} are

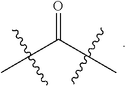 [SMILES: *C(*)=O]

In some variations of formula (25) described in the paragraphs above in which o₁ is 2, Rₐ₁ and R_{b1} are

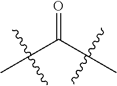 [SMILES: *C(*)=O]

Rₐ₂ and R_{b2} are

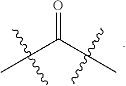 [SMILES: *C(*)=O]

In variations of formula (25) described in the paragraphs above in which o₁ is 3, the three instances of Rₐ and R_{b} are indicated as Rₐ₁ and R_{b1}; Rₐ₂ and R_{b2}; and Rₐ₃ and R_{b3}, respectively. In some variations of formula (25) described in the paragraphs above in which o₁ is 3, In some variations of formula (25) described in the paragraphs above in which o₁ is 3, each of Rₐ₁, R_{b1}, Rₐ₂, R_{b2}, Rₐ₃, and R_{b3} is H. In some variations of formula (25) described in the paragraphs above in which o₁ is 3, each of Rₐ₁, R_{b1}, Rₐ₂, R_{b2}, Rₐ₃, and R_{b3} is CH₃. In some variations of formula (25) described in the paragraphs above in which o₁ is 3, each of Rₐ₁, R_{b1}, Rₐ₂, and R_{b2}, is H and each of Rₐ₃ and R_{b3} is CH₃. In some variations of formula (25) described in the paragraphs above in which o₁ is 3, each of Rₐ₁, R_{b1}, Rₐ₂, and R_{b2}, is CH₃ and each of Rₐ₃ and R_{b3} is H. In some variations of formula (25) described in the paragraphs above in which o₁ is 3, Rₐ₁ is H and each of Rₐ₂, Rₐ₃, R_{b1}, R_{b2}, and R_{b3} is CH₃. In some variations of formula (25) described in the paragraphs above in which o₁ is 3, each of Rₐ₁ and Rₐ₂ is H and each of Rₐ₃, R_{b1}, R_{b2}, an R_{b3} is CH₃. In some variations of formula (25) described in the paragraphs above in which o₁ is 3, each of Rₐ₁, Rₐ₂, and Rₐ₃ is H and each of R_{b1}, R_{b2}, an R_{b3} is CH₃.

In some variations of formula (25) described in the paragraphs above in which o₁ is 3, each of Rₐ₁, R_{b1}, Rₐ₂, and R_{b2}, is H and Rₐ₃ and R_{b3} are

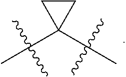 [SMILES: *C1(*)CC1]

In some variations of formula (25) described in the paragraphs above in which o₁ is 3, each of Rₐ₁, R_{b1}, Rₐ₂, and R_{b2}, is CH₃ and Rₐ₃ and R_{b3} are

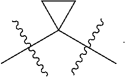 [SMILES: *C1(*)CC1]

In some variations of formula (25) described in the paragraphs above in which o₁ is 3, Rₐ₁ is H and each of Rₐ₂, R_{b1}, and R_{b2} is CH₃, and Rₐ₃ and R_{b3} are

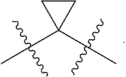 [SMILES: *C1(*)CC1]

In some variations of formula (25) described in the paragraphs above in which o₁ is 3, each of Rₐ₁ and Rₐ₂ is H and each of R_{b1} and R_{b2} is CH₃, and Rₐ₃ and R_{b3} are

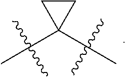 [SMILES: *C1(*)CC1]

In some variations of formula (25) described in the paragraphs above in which o₁ is 3, each of Rₐ₁, R_{b1}, Rₐ₂, and R_{b2}, is H and Rₐ₃ and R_{b3} are

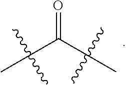 [SMILES: *C(*)=O]

In some variations of formula (25) described in the paragraphs above in which o₁ is 3, each of Rₐ₁, R_{b1}, Rₐ₂, and R_{b2}, is CH₃ and Rₐ₃ and R_{b3} are

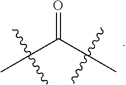 [SMILES: *C(*)=O]

In some variations of formula (25) described in the paragraphs above in which o₁ is 3, Rₐ₁ is H and each of Rₐ₂, R_{b1}, and R_{b2} is CH₃, and Rₐ₃ and R_{b3} are

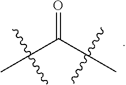 [SMILES: *C(*)=O]

In some variations of formula (25) described in the paragraphs above in which o₁ is 3, each of Rₐ₁ and Rₐ₂ is H and each of R_{b1} and R_{b2} is CH₃, and Rₐ₃ and R_{b3} are

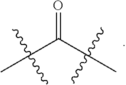 [SMILES: *C(*)=O]

In some variations of formula (25) described in the paragraphs above in which o₁ is 3, Rₐ₁ and R_{b1} are

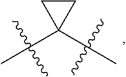 [SMILES: *C1(*)CC1]

Rₐ₂ and R_{b2} are

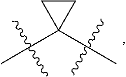 [SMILES: *C1(*)CC1]

and each of Rₐ₃ and R_{b3} is H. In some variations of formula (25) described in the paragraphs above in which o₁ is 3, Rₐ₁ and R_{b1} are

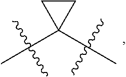 [SMILES: *C1(*)CC1]

Rₐ₂ and R_{b2} are

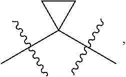 [SMILES: *C1(*)CC1]

and each of Rₐ₃ and R_{b3} is CH₃. In some variations of formula (25) described in the paragraphs above in which o₁ is 3, Rₐ₁ and R_{b1} are

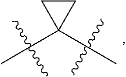 [SMILES: *C1(*)CC1]

Rₐ₂ and R_{b2} are

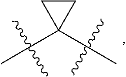 [SMILES: *C1(*)CC1]

Rₐ₃ is H, and R_{b3} is CH₃. In some variations of formula (25) described in the paragraphs above in which o₁ is 3, Rₐ₁ and R_{b1} are

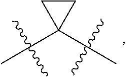 [SMILES: *C1(*)CC1]

Rₐ₂ and R_{b2} are

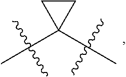 [SMILES: *C1(*)CC1]

and each of Rₐ₃ and R_{b3} is H. In some variations of formula (25) described in the paragraphs above in which o₁ is 3, Rₐ₁ and R_{b1} are

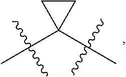 [SMILES: *C1(*)CC1]

Rₐ₂ and R_{b2} are

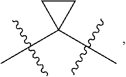 [SMILES: *C1(*)CC1]

and each of Rₐ₃ and R_{b3} is CH₃. In some variations of formula (25) described in the paragraphs above in which o₁ is 3, Rₐ₁ and R_{b1} are

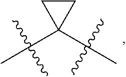 [SMILES: *C1(*)CC1]

Rₐ₂ and R_{b2} are

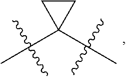 [SMILES: *C1(*)CC1]

Rₐ₃ is H, and R_{b3} is CH₃.

In some variations of formula (25) described in the paragraphs above in which o₁ is 3, Rₐ₁ and R_{b1} are

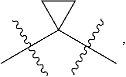 [SMILES: *C1(*)CC1]

Rₐ₂ and R_{b2} are

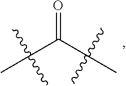 [SMILES: *C(*)=O]

and each of Rₐ₃ and R_{b3} is H. In some variations of formula (25) described in the paragraphs above in which o₁ is 3, Rₐ₁ and R_{b1} are

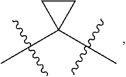 [SMILES: *C1(*)CC1]

Rₐ₂ and R_{b2} are

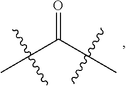 [SMILES: *C(*)=O]

and each of Rₐ₃ and R_{b3} is CH₃. In some variations of formula (25) described in the paragraphs above in which o₁ is 3, Rₐ₁ and R_{b1} are

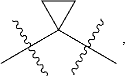 [SMILES: *C1(*)CC1]

Rₐ₂ and R_{b2} are

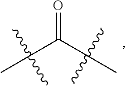 [SMILES: *C(*)=O]

Rₐ₃ is H, and R_{b3} is CH₃. In some variations of formula (25) described in the paragraphs above in which o₁ is 3, Rₐ₁ and R_{b1} are

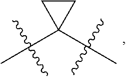 [SMILES: *C1(*)CC1]

Rₐ₂ and R_{b2} are

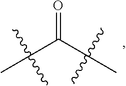 [SMILES: *C(*)=O]

and each of Rₐ₃ and R_{b3} is H. In some variations of formula (25) described in the paragraphs above in which o₁ is 3, Rₐ₁ and R_{b1} are

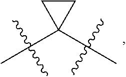 [SMILES: *C1(*)CC1]

Rₐ₂ and R_{b2} are

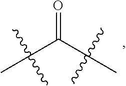 [SMILES: *C(*)=O]

and each of Rₐ₃ and R_{b3} is CH₃. In some variations of formula (25) described in the paragraphs above in which o₁ is 3, Rₐ₁ and R_{b1} are

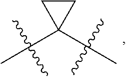 [SMILES: *C1(*)CC1]

Rₐ₂ and R_{b2} are

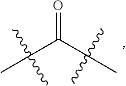 [SMILES: *C(*)=O]

Rₐ₃ is H, and R_{b3} is CH₃.

In some variations of formula (25) described in the paragraphs above in which o₁ is 3, Rₐ₁ and R_{b1} are

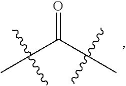 [SMILES: *C(*)=O]

Rₐ₂ and R_{b2} are

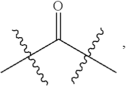 [SMILES: *C(*)=O]

and each of Rₐ₃ and R_{b3} is H. In some variations of formula (25) described in the paragraphs above in which o₁ is 3, Rₐ₁ and R_{b1} are

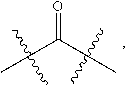 [SMILES: *C(*)=O]

Rₐ₂ and R_{b2} are

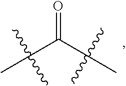 [SMILES: *C(*)=O]

and each of Rₐ₃ and R_{b3} is CH₃. In some variations of formula (25) described in the paragraphs above in which o₁ is 3, Rₐ₁ and R_{b1} are

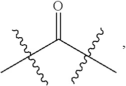 [SMILES: *C(*)=O]

Rₐ₂ and R_{b2} are

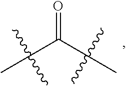 [SMILES: *C(*)=O]

Rₐ₃ is H, and R_{b3} is CH₃. In some variations of formula (25) described in the paragraphs above in which o₁ is 3, Rₐ₁ and R_{b1} are

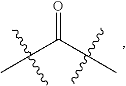 [SMILES: *C(*)=O]

Rₐ₂ and R_{b2} are

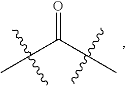 [SMILES: *C(*)=O]

and each of Rₐ₃ and R_{b3} is H. In some variations of formula (25) described in the paragraphs above in which o₁ is 3, Rₐ₁ and R_{b1} are

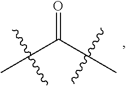 [SMILES: *C(*)=O]

Rₐ₂ and R_{b2} are

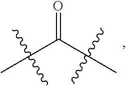 [SMILES: *C(*)=O]

and each of Rₐ₃ and R_{b3} is CH₃. In some variations of formula (25) described in the paragraphs above in which o₁ is 3, Rₐ₁ and R_{b1} are

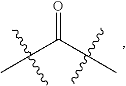 [SMILES: *C(*)=O]

Rₐ₂ and R_{b2} are

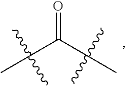 [SMILES: *C(*)=O]

Rₐ₃ is H, and R_{b3} is CH₃.

In some variations of formula (25) described in the paragraphs above in which o₁ is 3, Rₐ₁ and R_{b1} and Rₐ₂ and R_{b2} together are

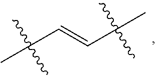 [SMILES: */C=C/*]

and each of Rₐ₃ and R_{b3} is H. In some variations of formula (25) described in the paragraphs above in which o₁ is 3, Rₐ₁ and R_{b1} and Rₐ₂ and R_{b2} together are

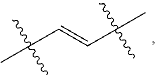 [SMILES: */C=C/*]

and each of Rₐ₃ and R_{b3} is CH₃. In some variations of formula (25) described in the paragraphs above in which o₁ is 3, Rₐ₁ and R_{b1} and Rₐ₂ and R_{b2} together are

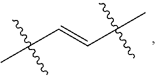 [SMILES: */C=C/*]

Rₐ₃ is H, and R_{b3} is CH₃. In some variations of formula (25) described in the paragraphs above in which o₁ is 3, Rₐ₁ and R_{b1} and Rₐ₂ and R_{b2} together are

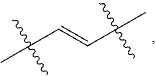 [SMILES: */C=C/*]

and Rₐ₃ and R_{b3} are

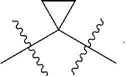 [SMILES: *C1(*)CC1]

In some variations of formula (25) described in the paragraphs above in which o₁ is 3, Rₐ₁ and R_{b1} and Rₐ₂ and R_{b2} together are

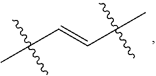 [SMILES: */C=C/*]

and Rₐ₃ and R_{b3} are

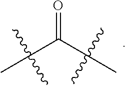 [SMILES: *C(*)=O]

In variations of formula (25) described in the paragraphs above in which o₁ is 4, the three instances of Rₐ and R_{b} are indicated as Rₐ₁ and R_{b1}; Rₐ₂ and R_{b2}; Rₐ₃ and R_{b3}; and Rₐ₄ and R_{b4}, respectively. In some variations of formula (25) described in the paragraphs above in which o₁ is 4, In some variations of formula (25) described in the paragraphs above in which o₁ is 4, each of Rₐ₁, R_{b1}, Rₐ₂, R_{b2}, Rₐ₃, R_{b3}, Rₐ₄, and R_{b4} is H. In some variations of formula (25) described in the paragraphs above in which o₁ is 4, each of Rₐ₁, R_{b1}, Rₐ₂, R_{b2}, Rₐ₃, R_{b3}, Rₐ₄, and R_{b4} is CH₃. In some variations of formula (25) described in the paragraphs above in which o₁ is 4, each of Rₐ₁, R_{b1}, Rₐ₂, R_{b2}, Rₐ₃, and R_{b3} is H and each of Rₐ₄ and R_{b4} is CH₃. In some variations of formula (25) described in the paragraphs above in which o₁ is 4, each of Rₐ₁, R_{b1}, Rₐ₂, and R_{b2} is H and each of Rₐ₃, R_{b3}, Rₐ₄, and R_{b4} is CH₃. In some variations of formula (25) described in the paragraphs above in which o₁ is 4, each of Rₐ₁, R_{b1}, Rₐ₂, R_{b2}, Rₐ₃, and R_{b3} is CH₃ and each of Rₐ₄ and R_{b4} is H.

In some variations of formula (25) described in the paragraphs above in which o₁ is 4, each of Rₐ₁, Rₐ₂, and Rₐ₃ is H and each of R_{b1}, R_{b2}, and R_{b3} is CH₃. In some variations of formula (25) described in the paragraphs above in which o₁ is 4, Rₐ₁ is H, R_{b1} is CH₃, and each of Rₐ₂, R_{b2}, Rₐ₃, and R_{b3} is CH₃. In some variations of formula (25) described in the paragraphs above in which o₁ is 4, Rₐ₁ is H, R_{b1} is CH₃, and each of Rₐ₂, R_{b2}, Rₐ₃, and R_{b3} is H. In some variations of formula (25) described in the paragraphs above in which o₁ is 4, each of Rₐ₁ and Rₐ₂ is H, each of R_{b1} and R_{b2} is CH₃, and each of Rₐ₃ and R_{b3} is H. In some variations of formula (25) described in the paragraphs above in which o₁ is 4, each of Rₐ₁ and Rₐ₂ is H, each of R_{b1} and R_{b2} is CH₃, and each of Rₐ₃ and R_{b3} is CH₃.

In some variations of formula (25) described in the paragraphs above in which o₁ is 4, Rₐ₁ and R_{b1} are H, Rₐ₂ and R_{b2} are H, Rₐ₃ and R_{b3} are H, and Rₐ₄ and R_{b4} are

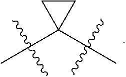 [SMILES: *C1(*)CC1]

In some variations of formula (25) described in the paragraphs above in which o₁ is 4, Rₐ₁ and R_{b1} are H, Rₐ₂ and R_{b2} are H, Rₐ₃ and R_{b3} are CH₃, and Rₐ₄ and R_{b4} are

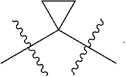 [SMILES: *C1(*)CC1]

In some variations of formula (25) described in the paragraphs above in which o₁ is 4, Rₐ₁ and R_{b1} are H, Rₐ₂ and R_{b2} are CH₃, Rₐ₃ and R_{b3} are CH₃, and Rₐ₄ and R_{b4} are

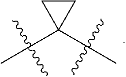 [SMILES: *C1(*)CC1]

In some variations of formula (25) described in the paragraphs above in which o₁ is 4, Rₐ₁ and R_{b1} are CH₃, Rₐ₂ and R_{b2} are CH₃, Rₐ₃ and R_{b3} are CH₃, and Rₐ₄ and R_{b4} are

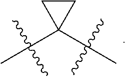 [SMILES: *C1(*)CC1]

In some variations of formula (25) described in the paragraphs above in which o₁ is 4, Rₐ₁ and R_{b1} are H, Rₐ₂ and R_{b2} are H, Rₐ₃ and R_{b3} are

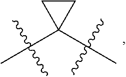 [SMILES: *C1(*)CC1]

and Rₐ₄ and R_{b4} are

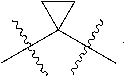 [SMILES: *C1(*)CC1]

In some variations of formula (25) described in the paragraphs above in which o₁ is 4, Rₐ₁ and R_{b1} are H, Rₐ₂ and R_{b2} are CH₃, Rₐ₃ and R_{b3} are

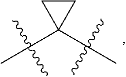 [SMILES: *C1(*)CC1]

and Rₐ₄ and R_{b4} are

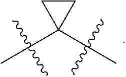 [SMILES: *C1(*)CC1]

In some variations of formula (25) described in the paragraphs above in which o₁ is 4, Rₐ₁ and R_{b1} are CH₃, Rₐ₂ and R_{b2} are CH₃, Rₐ₃ and R_{b3} are

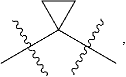 [SMILES: *C1(*)CC1]

and Rₐ₄ and R_{b4} are

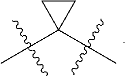 [SMILES: *C1(*)CC1]

In some variations of formula (25) described in the paragraphs above in which o₁ is 4, Rₐ₁ is H, R_{b1} is CH₃, Rₐ₂ and R_{b2} are CH₃, Rₐ₃ and R_{b3} are

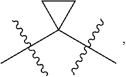 [SMILES: *C1(*)CC1]

and Rₐ₄ and R_{b4} are

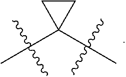 [SMILES: *C1(*)CC1]

In some variations of formula (25) described in the paragraphs above in which o₁ is 4, Rₐ₁ is H, R_{b1} is CH₃, Rₐ₂ is H, R_{b2} is CH₃, Rₐ₃ and R_{b3} are

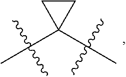 [SMILES: *C1(*)CC1]

and Rₐ₄ and R_{b4} are

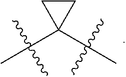 [SMILES: *C1(*)CC1]

In some variations of formula (25) described in the paragraphs above in which o₁ is 4, Rₐ₁ and R_{b1} are H, Rₐ₂ and R_{b2} are

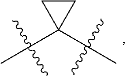 [SMILES: *C1(*)CC1]

Rₐ₃ and R_{b3} are

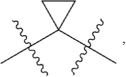 [SMILES: *C1(*)CC1]

and Rₐ₄ and R_{b4} are

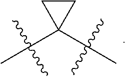 [SMILES: *C1(*)CC1]

In some variations of formula (25) described in the paragraphs above in which o₁ is 4, Rₐ₁ and R_{b1} are CH₃, Rₐ₂ and R_{b2} are

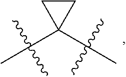 [SMILES: *C1(*)CC1]

Rₐ₃ and R_{b3} are

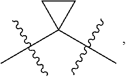 [SMILES: *C1(*)CC1]

and Rₐ₄ and R_{b4} are

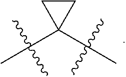 [SMILES: *C1(*)CC1]

In some variations of formula (25) described in the paragraphs above in which o₁ is 4, Rₐ₁ is H, R_{b1} is CH₃, Rₐ₂ and R_{b2} are

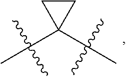 [SMILES: *C1(*)CC1]

Rₐ₃ and R_{b3} are

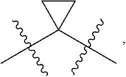 [SMILES: *C1(*)CC1]

and Rₐ₄ and R_{b4} are

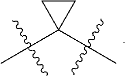 [SMILES: *C1(*)CC1]

In some variations of formula (25) described in the paragraphs above in which o₁ is 4, Rₐ₁ and R_{b1} are

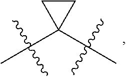 [SMILES: *C1(*)CC1]

Rₐ₂ and R_{b2} are

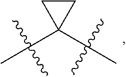 [SMILES: *C1(*)CC1]

Rₐ₃ and R_{b3} are

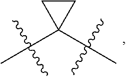 [SMILES: *C1(*)CC1]

and Rₐ₄ and R_{b4} are

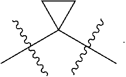 [SMILES: *C1(*)CC1]

In some variations of formula (25) described in the paragraphs above in which o₁ is 4, Rₐ₁ and R_{b1} are H, Rₐ₂ and R_{b2} are H, Rₐ₃ and R_{b3} are H, and Rₐ₄ and R_{b4} are

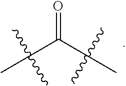 [SMILES: *C(*)=O]

In some variations of formula (25) described in the paragraphs above in which o₁ is 4, Rₐ₁ and R_{b1} are H, Rₐ₂ and R_{b2} are H, Rₐ₃ and R_{b3} are CH₃, and Rₐ₄ and R_{b4} are

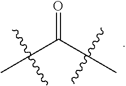 [SMILES: *C(*)=O]

In some variations of formula (25) described in the paragraphs above in which o₁ is 4, Rₐ₁ and R_{b1} are H, Rₐ₂ and R_{b2} are CH₃, Rₐ₃ and R_{b3} are CH₃, and Rₐ₄ and R_{b4} are

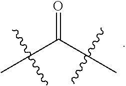 [SMILES: *C(*)=O]

In some variations of formula (25) described in the paragraphs above in which o₁ is 4, Rₐ₁ and R_{b1} are CH₃, Rₐ₂ and R_{b2} are CH₃, Rₐ₃ and R_{b3} are CH₃, and Rₐ₄ and R_{b4} are

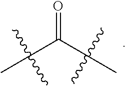 [SMILES: *C(*)=O]

In some variations of formula (25) described in the paragraphs above in which o₁ is 4, Rₐ₁ and R_{b1} are H, Rₐ₂ and R_{b2} are H, Rₐ₃ and R_{b3} are

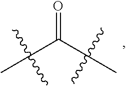 [SMILES: *C(*)=O]

and Rₐ₄ and R_{b4} are v. In some variations of formula (25) described in the paragraphs above in which o₁ is 4, Rₐ₁ and R_{b1} are H, Rₐ₂ and R_{b2} are CH₃, Rₐ₃ and R_{b3} are

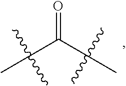 [SMILES: *C(*)=O]

and Rₐ₄ and R_{b4} are

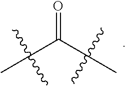 [SMILES: *C(*)=O]

In some variations of formula (25) described in the paragraphs above in which o₁ is 4, Rₐ₁ and R_{b1} are CH₃, Rₐ₂ and R_{b2} are CH₃, Rₐ₃ and R_{b3} are

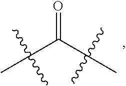 [SMILES: *C(*)=O]

and Rₐ₄ and R_{b4} are

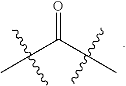 [SMILES: *C(*)=O]

In some variations of formula (25) described in the paragraphs above in which o₁ is 4, Rₐ₁ is H, R_{b1} is CH₃, Rₐ₂ and R_{b2} are CH₃, Rₐ₃ and R_{b3} are

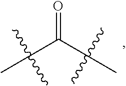 [SMILES: *C(*)=O]

and Rₐ₄ and R_{b4} are

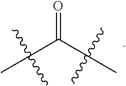 [SMILES: *C(*)=O]

In some variations of formula (25) described in the paragraphs above in which o₁ is 4, Rₐ₁ is H, R_{b1} is CH₃, Rₐ₂ is H, R_{b2} is CH₃, Rₐ₃ and R_{b3} are

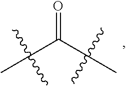 [SMILES: *C(*)=O]

and Rₐ₄ and R_{b4} are

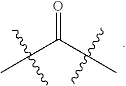 [SMILES: *C(*)=O]

In some variations of formula (25) described in the paragraphs above in which o₁ is 4, Rₐ₁ and R_{b1} are H, Rₐ₂ and R_{b2} are

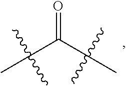 [SMILES: *C(*)=O]

Rₐ₃ and R_{b3} are

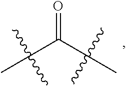 [SMILES: *C(*)=O]

and Rₐ₄ and R_{b4} are

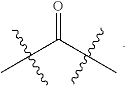 [SMILES: *C(*)=O]

In some variations of formula (25) described in the paragraphs above in which o₁ is 4, Rₐ₁ and R_{b1} are CH₃, Rₐ₂ and R_{b2} are

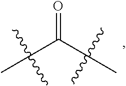 [SMILES: *C(*)=O]

Rₐ₃ and R_{b3} are

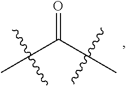 [SMILES: *C(*)=O]

and Rₐ₄ and R_{b4} are

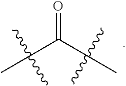 [SMILES: *C(*)=O]

In some variations of formula (25) described in the paragraphs above in which o₁ is 4, Rₐ₁ is H, R_{b1} is CH₃, Rₐ₂ and R_{b2} are

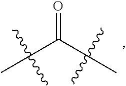 [SMILES: *C(*)=O]

Rₐ₃ and R_{b3} are

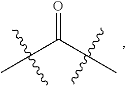 [SMILES: *C(*)=O]

and Rₐ₄ and R_{b4} are

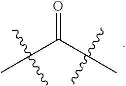 [SMILES: *C(*)=O]

In some variations of formula (25) described in the paragraphs above in which o₁ is 4, Rₐ₁ and R_{b1} are

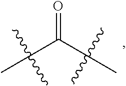 [SMILES: *C(*)=O]

Rₐ₂ and R_{b2} are

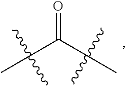 [SMILES: *C(*)=O]

Rₐ₃ and R_{b3} are

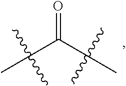 [SMILES: *C(*)=O]

and Rₐ₄ and R_{b4} are

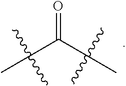 [SMILES: *C(*)=O]

In some variations of formula (25) described in the paragraphs above in which o₁ is 4, Rₐ₁ and R_{b1} are

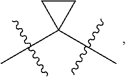 [SMILES: *C1(*)CC1]

Rₐ₂ and R_{b2} are H, Rₐ₃ and R_{b3} are H, and Rₐ₄ and R_{b4} are

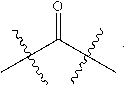 [SMILES: *C(*)=O]

In some variations of formula (25) described in the paragraphs above in which o₁ is 4, Rₐ₁ and R_{b1} are

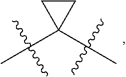 [SMILES: *C1(*)CC1]

Rₐ₂ and R_{b2} are CH₃, Rₐ₃ and R_{b3} are CH₃, and Rₐ₄ and R_{b4} are

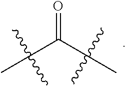 [SMILES: *C(*)=O]

In some variations of formula (25) described in the paragraphs above in which o₁ is 4, Rₐ₁ and R_{b1} are

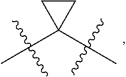 [SMILES: *C1(*)CC1]

Rₐ₂ and R_{b2} are H, Rₐ₃ and R_{b3} are CH₃, and Rₐ₄ and R_{b4} are

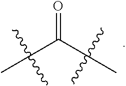 [SMILES: *C(*)=O]

In some variations of formula (25) described in the paragraphs above in which o₁ is 4, Rₐ₁ and R_{b1} are

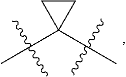 [SMILES: *C1(*)CC1]

Rₐ₂ and R_{b2} are

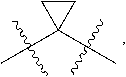 [SMILES: *C1(*)CC1]

Rₐ₃ and R_{b3} are H, and Rₐ₄ and R_{b4} are

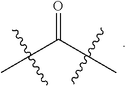 [SMILES: *C(*)=O]

In some variations of formula (25) described in the paragraphs above in which o₁ is 4, Rₐ₁ and R_{b1} are

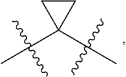 [SMILES: *C1(*)CC1]

Rₐ₂ and R_{b2} are

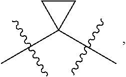 [SMILES: *C1(*)CC1]

Rₐ₃ and R_{b3} are CH₃, and Rₐ₄ and R_{b4} are

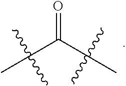 [SMILES: *C(*)=O]

In some variations of formula (25) described in the paragraphs above in which o₁ is 4, Rₐ₁ and R_{b1} are

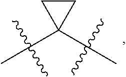 [SMILES: *C1(*)CC1]

Rₐ₂ and R_{b2} are

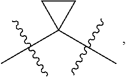 [SMILES: *C1(*)CC1]

Rₐ₃ and R_{b3} are CH₃, and Rₐ₄ and R_{b4} are

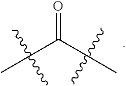 [SMILES: *C(*)=O]

In some variations of formula (25) described in the paragraphs above in which o₁ is 4, Rₐ₁ and R_{b1} are

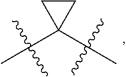 [SMILES: *C1(*)CC1]

Rₐ₂ and R_{b2} are

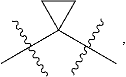 [SMILES: *C1(*)CC1]

Rₐ₃ and R_{b3} are H, and Rₐ₄ and R_{b4} are

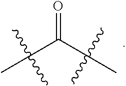 [SMILES: *C(*)=O]

In some variations of formula (25) described in the paragraphs above in which o₁ is 4, Rₐ₁ and R_{b1} are

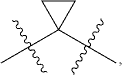 [SMILES: *C1(*)CC1]

Rₐ₂ and R_{b2} are

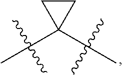 [SMILES: *C1(*)CC1]

Rₐ₃ and R_{b3} are

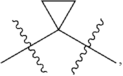 [SMILES: *C1(*)CC1]

and Rₐ₄ and R_{b4} are

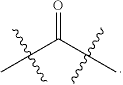 [SMILES: *C(*)=O]

In some variations of formula (25) described in the paragraphs above in which o₁ is 4, Rₐ₁ and R_{b1} are

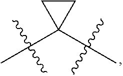 [SMILES: *C1(*)CC1]

Rₐ₂ and R_{b2} are

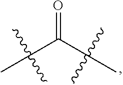 [SMILES: *C(*)=O]

Rₐ₃ and R_{b3} are H, and Rₐ₄ and R_{b4} are

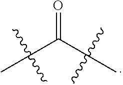 [SMILES: *C(*)=O]

In some variations of formula (25) described in the paragraphs above in which o₁ is 4, Rₐ₁ and R_{b1} are

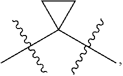 [SMILES: *C1(*)CC1]

Rₐ₂ and R_{b2} are

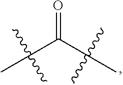 [SMILES: *C(*)=O]

Rₐ₃ and R_{b3} are CH₃, and Rₐ₄ and R_{b4} are

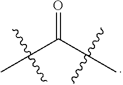 [SMILES: *C(*)=O]

In some variations of formula (25) described in the paragraphs above in which o₁ is 4, Rₐ₁ and R_{b1} are

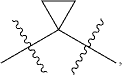 [SMILES: *C1(*)CC1]

Rₐ₂ and R_{b2} are

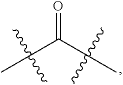 [SMILES: *C(*)=O]

Rₐ₃ and R_{b3} are CH₃, and Rₐ₄ and R_{b4} are

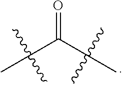 [SMILES: *C(*)=O]

In some variations of formula (25) described in the paragraphs above in which o₁ is 4, Rₐ₁ and R_{b1} are

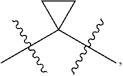 [SMILES: *C1(*)CC1]

Rₐ₂ and R_{b2} are

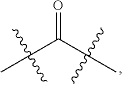 [SMILES: *C(*)=O]

Rₐ₃ and R_{b3} are H, and Rₐ₄ and R_{b4} are

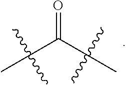 [SMILES: *C(*)=O]

In some variations of formula (25) described in the paragraphs above in which o₁ is 4, Rₐ₁ and R_{b1} are

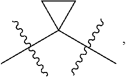 [SMILES: *C1(*)CC1]

Rₐ₂ and R_{b2} are

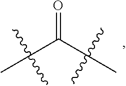 [SMILES: *C(*)=O]

Rₐ₃ and R_{b3} are

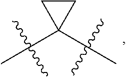 [SMILES: *C1(*)CC1]

and Rₐ₄ and R_{b4} are

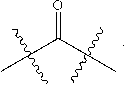 [SMILES: *C(*)=O]

In some variations of formula (25) described in the paragraphs above in which o₁ is 4, Rₐ₁ and R_{b1} are

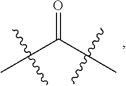 [SMILES: *C(*)=O]

Rₐ₂ and R_{b2} are

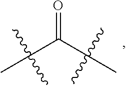 [SMILES: *C(*)=O]

Rₐ₃ and R_{b3} are H, and Rₐ₄ and R_{b4} are

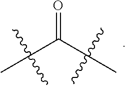 [SMILES: *C(*)=O]

In some variations of formula (25) described in the paragraphs above in which o₁ is 4, Rₐ₁ and R_{b1} are

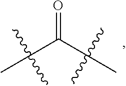 [SMILES: *C(*)=O]

Rₐ₂ and R_{b2} are

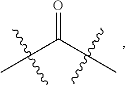 [SMILES: *C(*)=O]

Rₐ₃ and R_{b3} are CH₃, and Rₐ₄ and R_{b4} are

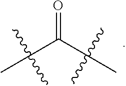 [SMILES: *C(*)=O]

In some variations of formula (25) described in the paragraphs above in which o₁ is 4, Rₐ₁ and R_{b1} are

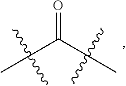 [SMILES: *C(*)=O]

Rₐ₂ and R_{b2} are

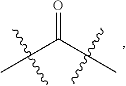 [SMILES: *C(*)=O]

Rₐ₃ and R_{b3} are CH₃, and Rₐ₄ and R_{b4} are

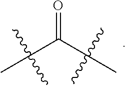 [SMILES: *C(*)=O]

In some variations of formula (25) described in the paragraphs above in which o₁ is 4, Rₐ₁ and R_{b1} are

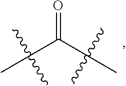 [SMILES: *C(*)=O]

Rₐ₂ and R_{b2} are

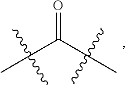 [SMILES: *C(*)=O]

Rₐ₃ and R_{b3} are H, and Rₐ₄ and R_{b4} are

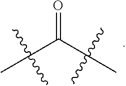 [SMILES: *C(*)=O]

In some variations of formula (25) described in the paragraphs above in which o₁ is 4, Rₐ₁ and R_{b1} are

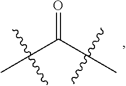 [SMILES: *C(*)=O]

Rₐ₂ and R_{b2} are

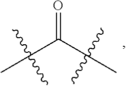 [SMILES: *C(*)=O]

Rₐ₃ and R_{b3} are

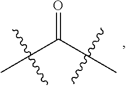 [SMILES: *C(*)=O]

and Rₐ₄ and R_{b4} are

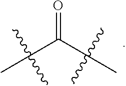 [SMILES: *C(*)=O]

In some variations of formula (25) described in the paragraphs above in which o₁ is 4, Rₐ₁ and R_{b1} are H, Rₐ₂ and R_{b2} are H, and Rₐ₃ and R_{b3} and Rₐ₄ and R_{b4} together are

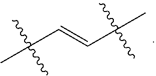 [SMILES: */C=C/*]

In some variations of formula (25) described in the paragraphs above in which o₁ is 4, Rₐ₁ and R_{b1} are H, Rₐ₂ and R_{b2} are CH₃, and Rₐ₃ and R_{b3} and Rₐ₄ and R_{b4} together are

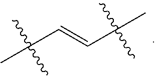 [SMILES: */C=C/*]

In some variations of formula (25) described in the paragraphs above in which o₁ is 4, Rₐ₁ and Rₐ₂ are H, R_{b1} and R_{b2} are CH₃, and Rₐ₃ and R_{b3} and Rₐ₄ and R_{b4} together are

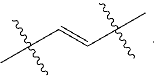 [SMILES: */C=C/*]

In some variations of formula (25) described in the paragraphs above in which o₁ is 4, Rₐ₁ and R_{b1} are H, Rₐ₂ and R_{b2} are

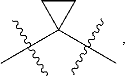 [SMILES: *C1(*)CC1]

and Rₐ₃ and R_{b3} and Rₐ₄ and R_{b4} together are

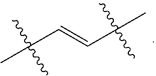 [SMILES: */C=C/*]

In some variations of formula (25) described in the paragraphs above in which o₁ is 4, Rₐ₁ and R_{b1} are

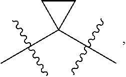 [SMILES: *C1(*)CC1]

Rₐ₂ and R_{b2} are CH₃, and Rₐ₃ and R_{b3} and Rₐ₄ and R_{b4} together are

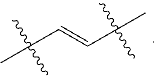 [SMILES: */C=C/*]

In some variations of formula (25) described in the paragraphs above in which o₁ is 4, Rₐ₁ is H, R_{b1} is CH₃, Rₐ₂ and R_{b2} are

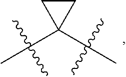 [SMILES: *C1(*)CC1]

and Rₐ₃ and R_{b3} and Rₐ₄ and R_{b4} together are

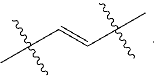 [SMILES: */C=C/*]

In some variations of formula (25) described in the paragraphs above in which o₁ is 4, Rₐ₁ and R_{b1} are H, Rₐ₂ and R_{b2} are

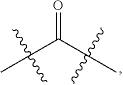 [SMILES: *C(*)=O]

and Rₐ₃ and R_{b3} and Rₐ₄ and R_{b4} together are

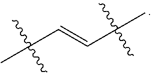 [SMILES: */C=C/*]

In some variations of formula (25) described in the paragraphs above in which o₁ is 4, Rₐ₁ and R_{b1} are

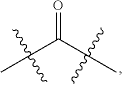 [SMILES: *C(*)=O]

Rₐ₂ and R_{b2} are CH₃, and Rₐ₃ and R_{b3} and Rₐ₄ and R_{b4} together are

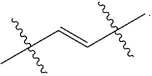 [SMILES: */C=C/*]

In some variations of formula (25) described in the paragraphs above in which o₁ is 4, Rₐ₁ is H, R_{b1} is CH₃, Rₐ₂ and R_{b2} are

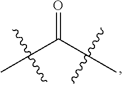 [SMILES: *C(*)=O]

and Rₐ₃ and R_{b3} and Rₐ₄ and R_{b4} together are

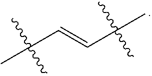 [SMILES: */C=C/*]

In some variations of formula (25) described in the paragraphs above in which o₁ is 4, Rₐ₁ and R_{b1} are

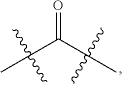 [SMILES: *C(*)=O]

Rₐ₂ and R_{b2} are

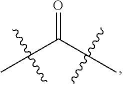 [SMILES: *C(*)=O]

and Rₐ₃ and R_{b3} and Rₐ₄ and R_{b4} together are

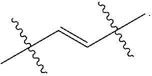 [SMILES: */C=C/*]

In some variations of formula (25) described in the paragraphs above in which o₁ is 4, Rₐ₁ and R_{b1} are

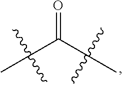 [SMILES: *C(*)=O]

Rₐ₂ and R_{b2} are

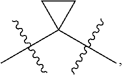 [SMILES: *C1(*)CC1]

and Rₐ₃ and R_{b3} and Rₐ₄ and R_{b4} together are

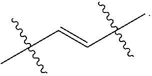 [SMILES: */C=C/*]

In some variations of formula (25) described in the paragraphs above in which o₁ is 4, Rₐ₁ and R_{b1} are

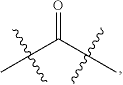 [SMILES: *C(*)=O]

Rₐ₂ and R_{b2} are

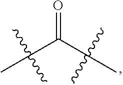 [SMILES: *C(*)=O]

and Rₐ₃ and R_{b3} and Rₐ₄ and R_{b4} together are

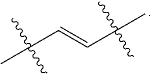 [SMILES: */C=C/*]

In some variations of formula (25) described in the paragraphs above in which o₁ is 4, Rₐ₁ and R_{b1} are

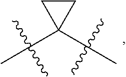 [SMILES: *C1(*)CC1]

Rₐ₂ and R_{b2} are

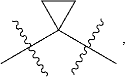 [SMILES: *C1(*)CC1]

and Rₐ₃ and R_{b3} and Rₐ₄ and R_{b4} together are

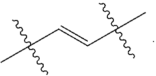 [SMILES: */C=C/*]

In some variations of formula (25) described in the paragraphs above in which o₁ is 4, Rₐ₁ and R_{b1} and Rₐ₂ and R_{b2} together are

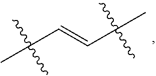 [SMILES: */C=C/*]

and Rₐ₃ and R_{b3} and Rₐ₄ and R_{b4} together are

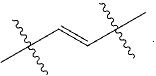 [SMILES: */C=C/*]

In some variations of formula (25) described in the paragraphs above in which o₂ is 1, Rₐ₁ and R_{b1} are both H. In some variations of formula (25) described in the paragraphs above in which o₂ is 1, Rₐ₁ is H and R_{b1} is CH₃. In some variations of formula (25) described in the paragraphs above in which o₂ is 1, Rₐ₁ and R_{b1} are both CH₃. In some variations of formula (25) described in the paragraphs above in which o₂ is 1, Rₐ₁ and R_{b1} are

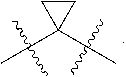 [SMILES: *C1(*)CC1]

In some variations of formula (25) described in the paragraphs above in which o₂ is 1, Rₐ₁ and R_{b1} are

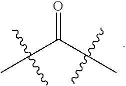 [SMILES: *C(*)=O]

In variations of formula (25) described in the paragraphs above in which o₂ is 2, the two instances of Rₐ and R_{b} are indicated as Rₐ₁ and R_{b1} and Rₐ₂ and R_{b2}, respectively. In some variations of formula (25) described in the paragraphs above in which o₂ is 2, each of Rₐ₁ and R_{b1} and Rₐ₂ and R_{b2} is H. In some variations of formula (25) described in the paragraphs above in which o₂ is 2, each of Rₐ₁ and R_{b1} and Rₐ₂ and R_{b2} is CH₃. In some variations of formula (25) described in the paragraphs above in which o₂ is 2, each of Rₐ₁ and Rₐ₂ is H and each of R_{b1} and R_{b2} is CH₃. In some variations of formula (25) described in the paragraphs above in which o₂ is 2, each of Rₐ₁, Rₐₑ, and R_{b1} is H and R_{b2} is CH₃.

In some variations of formula (25) described in the paragraphs above in which o₂ is 2, each of Rₐ₁ and R_{b1} is H and Rₐ₂ and R_{b2} are

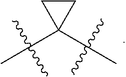 [SMILES: *C1(*)CC1]

In some variations of formula (25) described in the paragraphs above in which o₂ is 2, each of Rₐ₁ and R_{b1} is H and Rₐ₂ and R_{b2} are

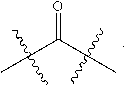 [SMILES: *C(*)=O]

In some variations of formula (25) described in the paragraphs above in which o₂ is 2, each of Rₐ₁ and R_{b1} is CH₃ and Rₐ₂ and R_{b2} are

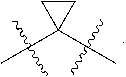 [SMILES: *C1(*)CC1]

In some variations of formula (25) described in the paragraphs above in which o₂ is 2, each of Rₐ₁ and R_{b1} is CH₃ and Rₐ₂ and R_{b2} are

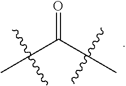 [SMILES: *C(*)=O]

In some variations of formula (25) described in the paragraphs above in which o₂ is 2, Rₐ₁ is H, R_{b1} is CH₃, and Rₐ₂ and R_{b2} are

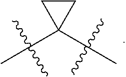 [SMILES: *C1(*)CC1]

In some variations of formula (25) described in the paragraphs above in which o₂ is 2, Rₐ₁ is H, R_{b1} is CH₃, and Rₐ₂ and R_{b2} are

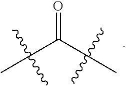 [SMILES: *C(*)=O]

In some variations of formula (25) described in the paragraphs above in which o₂ is 2, Rₐ₁ and R_{b1} and Rₐ₂ and R_{b2} together are

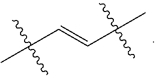 [SMILES: */C=C/*]

In some variations of formula (25) described in the paragraphs above in which o₂ is 2, Rₐ₁ and R_{b1} are

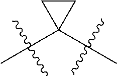 [SMILES: *C1(*)CC1]

Rₐ₂ and R_{b2} are

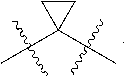 [SMILES: *C1(*)CC1]

In some variations of formula (25) described in the paragraphs above in which o₂ is 2, Rₐ₁ and R_{b1} are

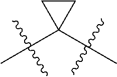 [SMILES: *C1(*)CC1]

Rₐ₂ and R_{b2} are

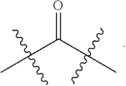 [SMILES: *C(*)=O]

In some variations of formula (25) described in the paragraphs above in which o₂ is 2, Rₐ₁ and R_{b1} are

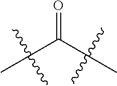 [SMILES: *C(*)=O]

Rₐ₂ and R_{b2} are

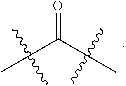 [SMILES: *C(*)=O]

In variations of formula (25) described in the paragraphs above in which o₂ is 3, the three instances of Rₐ and R_{b} are indicated as Rₐ₁ and R_{b1}; Rₐ₂ and R_{b2}; and Rₐ₃ and R_{b3}, respectively. In some variations of formula (25) described in the paragraphs above in which o₂ is 3, In some variations of formula (25) described in the paragraphs above in which o₂ is 3, each of Rₐ₁, R_{b1}, Rₐ₂, R_{b2}, Rₐ₃, and R_{b3} is H. In some variations of formula (25) described in the paragraphs above in which o₂ is 3, each of Rₐ₁, R_{b1}, Rₐ₂, R_{b2}, Rₐ₃, and R_{b3} is CH₃. In some variations of formula (25) described in the paragraphs above in which o₂ is 3, each of Rₐ₁, R_{b1}, Rₐ₂, an R_{b2}, is H and each of Rₐ₃ and R_{b3} is CH₃. In some variations of formula (25) described in the paragraphs above in which o₂ is 3, each of Rₐ₁, R_{b1}, Rₐ₂, and R_{b2}, is CH₃ and each of Rₐ₃ and R_{b3} is H. In some variations of formula (25) described in the paragraphs above in which o₂ is 3, Rₐ₁ is H and each of Rₐ₂, Rₐ₃, R_{b1}, R_{b2}, and R_{b3} is CH₃. In some variations of formula (25) described in the paragraphs above in which o₂ is 3, each of Rₐ₁ and Rₐ₂ is H and each of Rₐ₃, R_{b1}, R_{b2}, and R_{b3} is CH₃. In some variations of formula (25) described in the paragraphs above in which o₂ is 3, each of Rₐ₁, Rₐ₂, and Rₐ₃ is H and each of R_{b1}, R_{b2}, an R_{b3} is CH₃.

In some variations of formula (25) described in the paragraphs above in which o₂ is 3, each of Rₐ₁, R_{b1}, Rₐ₂, and R_{b2}, is H and Rₐ₃ and R_{b3} are

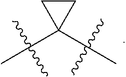 [SMILES: *C1(*)CC1]

In some variations of formula (25) described in the paragraphs above in which o₂ is 3, each of Rₐ₁, R_{b1}, Rₐ₂, and R_{b2}, is CH₃ and Rₐ₃ and R_{b3} are

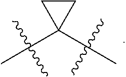 [SMILES: *C1(*)CC1]

In some variations of formula (25) described in the paragraphs above in which o₂ is 3, Rₐ₁ is H and each of Rₐ₂, R_{b1}, and R_{b2} is CH₃, and Rₐ₃ and R_{b3} are

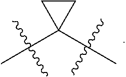 [SMILES: *C1(*)CC1]

In some variations of formula (25) described in the paragraphs above in which o₂ is 3, each of Rₐ₁ and Rₐ₂ is H and each of R_{b1} and R_{b2} is CH₃, and Rₐ₃ and R_{b3} are

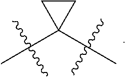 [SMILES: *C1(*)CC1]

In some variations of formula (25) described in the paragraphs above in which o₂ is 3, each of Rₐ₁, R_{b1}, Rₐ₂, and R_{b2}, is H and Rₐ₃ and R_{b3} are

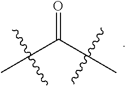 [SMILES: *C(*)=O]

In some variations of formula (25) described in the paragraphs above in which o₂ is 3, each of Rₐ₁, R_{b1}, Rₐ₂, and R_{b2}, is CH₃ and Rₐ₃ and R_{b3} are

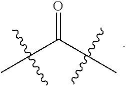 [SMILES: *C(*)=O]

In some variations of formula (25) described in the paragraphs above in which o₂ is 3, Rₐ₁ is H and each of Rₐ₂, R_{b1}, and R_{b2} is CH₃, and Rₐ₃ and R_{b3} are

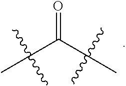 [SMILES: *C(*)=O]

In some variations of formula (25) described in the paragraphs above in which o₂ is 3, each of Rₐ₁ and Rₐ₂ is H and each of R_{b1} and R_{b2} is CH₃, and Rₐ₃ and R_{b3} are

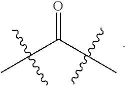 [SMILES: *C(*)=O]

In some variations of formula (25) described in the paragraphs above in which o₂ is 3, Rₐ₁ and R_{b1} are

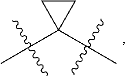 [SMILES: *C1(*)CC1]

Rₐ₂ and R_{b2} are

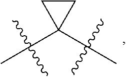 [SMILES: *C1(*)CC1]

and each of Rₐ₃ and R_{b3} is H. In some variations of formula (25) described in the paragraphs above in which o₂ is 3, Rₐ₁ and R_{b1} are

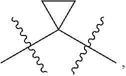 [SMILES: *C1(*)CC1]

Rₐ₂ and R_{b2} are

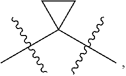 [SMILES: *C1(*)CC1]

and each of Rₐ₃ and R_{b3} is CH₃. In some variations of formula (25) described in the paragraphs above in which o₂ is 3, Rₐ₁ and R_{b1} are

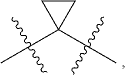 [SMILES: *C1(*)CC1]

Rₐ₂ and R_{b2} are

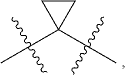 [SMILES: *C1(*)CC1]

Rₐ₃ is H, and R_{b3} is CH₃. In some variations of formula (25) described in the paragraphs above in which o₂ is 3, Rₐ₁ and R_{b1} are

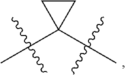 [SMILES: *C1(*)CC1]

Rₐ₂ and R_{b2} are

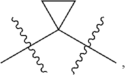 [SMILES: *C1(*)CC1]

and each of Rₐ₃ and R_{b3} is H. In some variations of formula (25) described in the paragraphs above in which o₂ is 3, Rₐ₁ and R_{b1} are

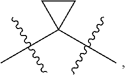 [SMILES: *C1(*)CC1]

Rₐ₂ and R_{b2} are

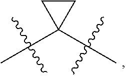 [SMILES: *C1(*)CC1]

and each of Rₐ₃ and R_{b3} is CH₃. In some variations of formula (25) described in the paragraphs above in which o₂ is 3, Rₐ₁ and R_{b1} are

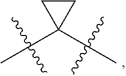 [SMILES: *C1(*)CC1]

Rₐ₂ and R_{b2} are

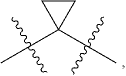 [SMILES: *C1(*)CC1]

Rₐ₃ is H, and R_{b3} is CH₃.

In some variations of formula (25) described in the paragraphs above in which o₂ is 3, Rₐ₁ and R_{b1} are

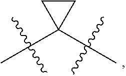 [SMILES: *C1(*)CC1]

Rₐ₂ and R_{b2} are

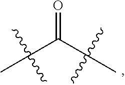 [SMILES: *C(*)=O]

and each of Rₐ₃ and R_{b3} is H. In some variations of formula (25) described in the paragraphs above in which o₂ is 3, Rₐ₁ and R_{b1} are

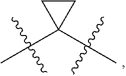 [SMILES: *C1(*)CC1]

Rₐ₂ and R_{b2} are

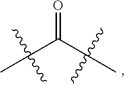 [SMILES: *C(*)=O]

and each of Rₐ₃ and R_{b3} is CH₃. In some variations of formula (25) described in the paragraphs above in which o₂ is 3, Rₐ₁ and R_{b1} are

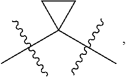 [SMILES: *C1(*)CC1]

Rₐ₂ and R_{b2} are

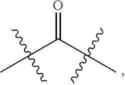 [SMILES: *C(*)=O]

Rₐ₃ is H, and R_{b3} is CH₃. In some variations of formula (25) described in the paragraphs above in which o₂ is 3, Rₐ₁ and R_{b1} are

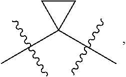 [SMILES: *C1(*)CC1]

Rₐ₂ and R_{b2} are

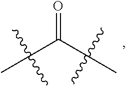 [SMILES: *C(*)=O]

and each of Rₐ₃ and R_{b3} is H. In some variations of formula (25) described in the paragraphs above in which o₂ is 3, Rₐ₁ and R_{b1} are

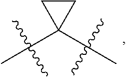 [SMILES: *C1(*)CC1]

Rₐ₂ and R_{b2} are

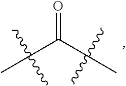 [SMILES: *C(*)=O]

and each of Rₐ₃ and R_{b3} is CH₃. In some variations of formula (25) described in the paragraphs above in which o₂ is 3, Rₐ₁ and R_{b1} are

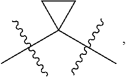 [SMILES: *C1(*)CC1]

Rₐ₂ and R_{b2} are

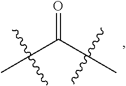 [SMILES: *C(*)=O]

Rₐ₃ is H, and R_{b3} is CH₃.

In some variations of formula (25) described in the paragraphs above in which o₂ is 3, Rₐ₁ and R_{b1} are

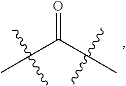 [SMILES: *C(*)=O]

Rₐ₂ and R_{b2} are

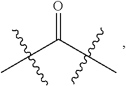 [SMILES: *C(*)=O]

and each of Rₐ₃ and R_{b3} is H. In some variations of formula (25) described in the paragraphs above in which o₂ is 3, Rₐ₁ and R_{b1} are

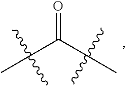 [SMILES: *C(*)=O]

Rₐ₂ and R_{b2} are

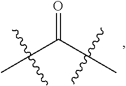 [SMILES: *C(*)=O]

and each of Rₐ₃ and R_{b3} is CH₃. In some variations of formula (25) described in the paragraphs above in which o₂ is 3, Rₐ₁ and R_{b1} are

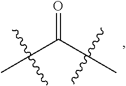 [SMILES: *C(*)=O]

Rₐ₂ and R_{b2} are

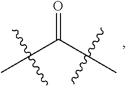 [SMILES: *C(*)=O]

Rₐ₃ is H, and R_{b3} is CH₃. In some variations of formula (25) described in the paragraphs above in which o₂ is 3, Rₐ₁ and R_{b1} are

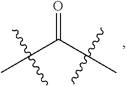 [SMILES: *C(*)=O]

Rₐ₂ and R_{b2} are

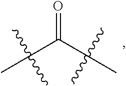 [SMILES: *C(*)=O]

and each of Rₐ₃ and R_{b3} is H. In some variations of formula (25) described in the paragraphs above in which o₂ is 3, Rₐ₁ and R_{b1} are

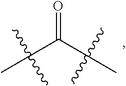 [SMILES: *C(*)=O]

Rₐ₂ and R_{b2} are

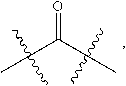 [SMILES: *C(*)=O]

and each of Rₐ₃ and R_{b3} is CH₃. In some variations of formula (25) described in the paragraphs above in which o₂ is 3, Rₐ₁ and R_{b1} are

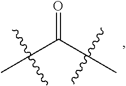 [SMILES: *C(*)=O]

Rₐ₂ and R_{b2} are

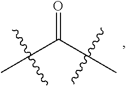 [SMILES: *C(*)=O]

Rₐ₃ is H, and R_{b3} is CH₃.

In some variations of formula (25) described in the paragraphs above in which o₂ is 3, Rₐ₁ and R_{b1} and Rₐ₂ and R_{b2} together are

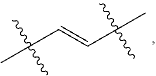 [SMILES: */C=C/*]

and each of Rₐ₃ and R_{b3} is H. In some variations of formula (25) described in the paragraphs above in which o₂ is 3, Rₐ₁ and R_{b1} and Rₐ₂ and R_{b2} together are

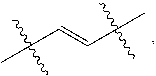 [SMILES: */C=C/*]

and each of Rₐ₃ and R_{b3} is CH₃. In some variations of formula (25) described in the paragraphs above in which o₂ is 3, Rₐ₁ and R_{b1} and Rₐ₂ and R_{b2} together are

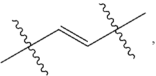 [SMILES: */C=C/*]

Rₐ₃ is H, and R_{b3} is CH₃. In some variations of formula (25) described in the paragraphs above in which o₂ is 3, Rₐ₁ and R_{b1} and Rₐ₂ and R_{b2} together are

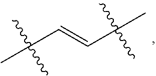 [SMILES: */C=C/*]

and Rₐ₃ and R_{b3} are

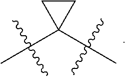 [SMILES: *C1(*)CC1]

In some variations of formula (25) described in the paragraphs above in which o₂ is 3, Rₐ₁ and R_{b1} and Rₐ₂ and R_{b2} together are

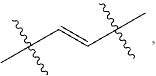 [SMILES: */C=C/*]

and Rₐ₃ and R_{b3} are

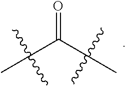 [SMILES: *C(*)=O]

In variations of formula (25) described in the paragraphs above in which o₂ is 4, the three instances of Rₐ and R_{b} are indicated as Rₐ₁ and R_{b1}; Rₐ₂ and R_{b2}; Rₐ₃ and R_{b3}; and Rₐ₄ and R_{b4}, respectively. In some variations of formula (25) described in the paragraphs above in which o₂ is 4, In some variations of formula (25) described in the paragraphs above in which o₂ is 4, each of Rₐ₁, R_{b1}, Rₐ₂, R_{b2}, Rₐ₃, R_{b3}, Rₐ₄, and R_{b4} is H. In some variations of formula (25) described in the paragraphs above in which o₂ is 4, each of Rₐ₁, R_{b1}, Rₐ₂, R_{b2}, Rₐ₃, R_{b3}, Rₐ₄, and R_{b4} is CH₃. In some variations of formula (25) described in the paragraphs above in which o₂ is 4, each of Rₐ₁, R_{b1}, Rₐ₂, R_{b2}, Rₐ₃, and R_{b3} is H and each of Rₐ₄ and R_{b4} is CH₃. In some variations of formula (25) described in the paragraphs above in which o₂ is 4, each of Rₐ₁, R_{b1}, Rₐ₂, and R_{b2} is H and each of Rₐ₃, R_{b3}, Rₐ₄, and R_{b4} is CH₃. In some variations of formula (25) described in the paragraphs above in which o₂ is 4, each of Rₐ₁, R_{b1}, Rₐ₂, R_{b2}, Rₐ₃, and R_{b3} is CH₃ and each of Rₐ₄ and R_{b4} is H.

In some variations of formula (25) described in the paragraphs above in which o₂ is 4, each of Rₐ₁, R_{b1}, and Rₐ₃ is H and each of R_{b1}, R_{b2}, and R_{b3} is CH₃. In some variations of formula (25) described in the paragraphs above in which o₂ is 4, Rₐ₁ is H, R_{b1} is CH₃, and each of Rₐ₂, R_{b2}, Rₐ₃, and R_{b3} is CH₃. In some variations of formula (25) described in the paragraphs above in which o₂ is 4, Rₐ₁ is H, R_{b1} is CH₃, and each of Rₐ₂, R_{b2}, Rₐ₃, and R_{b3} is H. In some variations of formula (25) described in the paragraphs above in which O₂ is 4, each of Rₐ₁ and Rₐ₂ is H, each of R_{b1} and R_{b2} is CH₃, and each of Rₐ₃ and R_{b3} is H. In some variations of formula (25) described in the paragraphs above in which o₂ is 4, each of Rₐ₁ and Rₐ₂ is H, each of R_{b1} and R_{b2} is CH₃, and each of Rₐ₃ and R_{b3} is CH₃.

In some variations of formula (25) described in the paragraphs above in which o₂ is 4, Rₐ₁ and R_{b1} are H, Rₐ₂ and R_{b2} are H, Rₐ₃ and R_{b3} are H, and Rₐ₄ and R_{b4} are

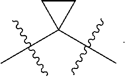 [SMILES: *C1(*)CC1]

In some variations of formula (25) described in the paragraphs above in which o₂ is 4, Rₐ₁ and R_{b1} are H, Rₐ₂ and R_{b2} are H, Rₐ₃ and R_{b3} are CH₃, and Rₐ₄ and R_{b4} are

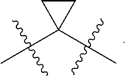 [SMILES: *C1(*)CC1]

In some variations of formula (25) described in the paragraphs above in which o₂ is 4, Rₐ₁ and R_{b1} are H, Rₐ₂ and R_{b2} are CH₃, Rₐ₃ and R_{b3} are CH₃, and Rₐ₄ and R_{b4} are

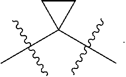 [SMILES: *C1(*)CC1]

In some variations of formula (25) described in the paragraphs above in which o₂ is 4, Rₐ₁ and R_{b1} are CH₃, Rₐ₂ and R_{b2} are CH₃, Rₐ₃ and R_{b3} are CH₃, and Rₐ₄ and R_{b4} are

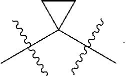 [SMILES: *C1(*)CC1]

In some variations of formula (25) described in the paragraphs above in which o₂ is 4, Rₐ₁ and R_{b1} are H, Rₐ₂ and R_{b2} are H, Rₐ₃ and R_{b3} are

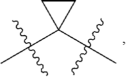 [SMILES: *C1(*)CC1]

and Rₐ₄ and R_{b4} are

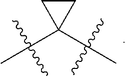 [SMILES: *C1(*)CC1]

In some variations of formula (25) described in the paragraphs above in which o₂ is 4, Rₐ₁ and R_{b1} are H, Rₐ₂ and R_{b2} are CH₃, Rₐ₃ and R_{b3} are

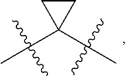 [SMILES: *C1(*)CC1]

and Rₐ₄ and R_{b4} are

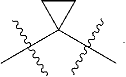 [SMILES: *C1(*)CC1]

In some variations of formula (25) described in the paragraphs above in which o₂ is 4, Rₐ₁ and R_{b1} are CH₃, Rₐ₂ and R_{b2} are CH₃, Rₐ₃ and R_{b3} are

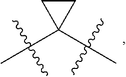 [SMILES: *C1(*)CC1]

and Rₐ₄ and R_{b4} are

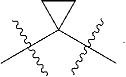 [SMILES: *C1(*)CC1]

In some variations of formula (25) described in the paragraphs above in which o₂ is 4, Rₐ₁ is H, R_{b1} is CH₃, Rₐ₂ and R_{b2} are CH₃, Rₐ₃ and R_{b3} are

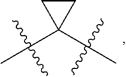 [SMILES: *C1(*)CC1]

and Rₐ₄ and R_{b4} are

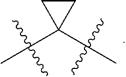 [SMILES: *C1(*)CC1]

In some variations of formula (25) described in the paragraphs above in which o₂ is 4, Rₐ₁ is H, R_{b1} is CH₃, Rₐ₂ is H, R_{b2} is CH₃, Rₐ₃ and R_{b3} are

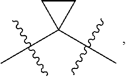 [SMILES: *C1(*)CC1]

and Rₐ₄ and R_{b4} are

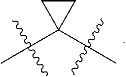 [SMILES: *C1(*)CC1]

In some variations of formula (25) described in the paragraphs above in which o₂ is 4, Rₐ₁ and R_{b1} are H, Rₐ₂ and R_{b2} are

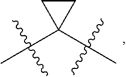 [SMILES: *C1(*)CC1]

Rₐ₃ and R_{b3} are

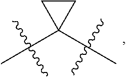 [SMILES: *C1(*)CC1]

and Rₐ₄ and R_{b4} are

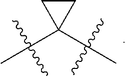 [SMILES: *C1(*)CC1]

In some variations of formula (25) described in the paragraphs above in which o₂ is 4, Rₐ₁ and R_{b1} are CH₃, Rₐ₂ and R_{b2} are

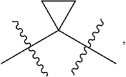 [SMILES: *C1(*)CC1]

Rₐ₃ and R_{b3} are

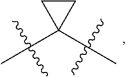 [SMILES: *C1(*)CC1]

and Rₐ₄ and R_{b4} are

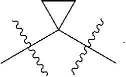 [SMILES: *C1(*)CC1]

In some variations of formula (25) described in the paragraphs above in which o₂ is 4, Rₐ₁ is H, R_{b1} is CH₃, Rₐ₂ and R_{b2} are

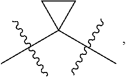 [SMILES: *C1(*)CC1]

Rₐ₃ and R_{b3} are

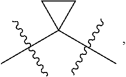 [SMILES: *C1(*)CC1]

and Rₐ₄ and R_{b4} are

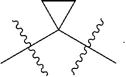 [SMILES: *C1(*)CC1]

In some variations of formula (25) described in the paragraphs above in which o₂ is 4, Rₐ₁ and R_{b1} are

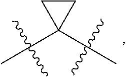 [SMILES: *C1(*)CC1]

Rₐ₂ and R_{b2} are

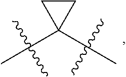 [SMILES: *C1(*)CC1]

Rₐ₃ and R_{b3} are

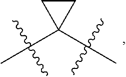 [SMILES: *C1(*)CC1]

and Rₐ₄ and R_{b4} are

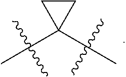 [SMILES: *C1(*)CC1]

In some variations of formula (25) described in the paragraphs above in which o₂ is 4, Rₐ₁ and R_{b1} are H, Rₐ₂ and R_{b2} are H, Rₐ₃ and R_{b3} are H, and Rₐ₄ and R_{b4} are

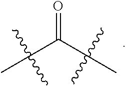 [SMILES: *C(*)=O]

In some variations of formula (25) described in the paragraphs above in which o₂ is 4, Rₐ₁ and R_{b1} are H, Rₐ₂ and R_{b2} are H, Rₐ₃ and R_{b3} are CH₃, and Rₐ₄ and R_{b4} are

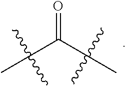 [SMILES: *C(*)=O]

In some variations of formula (25) described in the paragraphs above in which o₂ is 4, Rₐ₁ and R_{b1} are H, Rₐ₂ and R_{b2} are CH₃, Rₐ₃ and R_{b3} are CH₃, and Rₐ₄ and R_{b4} are

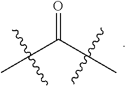 [SMILES: *C(*)=O]

In some variations of formula (25) described in the paragraphs above in which o₂ is 4, Rₐ₁ and R_{b1} are CH₃, Rₐ₂ and R_{b2} are CH₃, Rₐ₃ and R_{b3} are CH₃, and Rₐ₄ and R_{b4} are

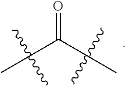 [SMILES: *C(*)=O]

In some variations of formula (25) described in the paragraphs above in which o₂ is 4, Rₐ₁ and R_{b1} are H, Rₐ₂ and R_{b2} are H, Rₐ₃ and R_{b3} are

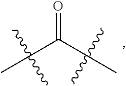 [SMILES: *C(*)=O]

and Rₐ₄ and R_{b4} are v. In some variations of formula (25) described in the paragraphs above in which o₂ is 4, Rₐ₁ and R_{b1} are H, Rₐ₂ and R_{b2} are CH₃, Rₐ₃ and R_{b3} are

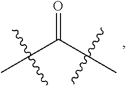 [SMILES: *C(*)=O]

and Rₐ₄ and R_{b4} are

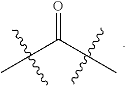 [SMILES: *C(*)=O]

In some variations of formula (25) described in the paragraphs above in which o₂ is 4, Rₐ₁ and R_{b1} are CH₃, Rₐ₂ and R_{b2} are CH₃, Rₐ₃ and R_{b3} are

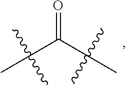 [SMILES: *C(*)=O]

and Rₐ₄ and R_{b4} are

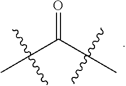 [SMILES: *C(*)=O]

In some variations of formula (25) described in the paragraphs above in which o₂ is 4, Rₐ₁ is H, R_{b1} is CH₃, Rₐ₂ and R_{b2} are CH₃, Rₐ₃ and R_{b3} are

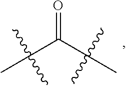 [SMILES: *C(*)=O]

and Rₐ₄ and R_{b4} are

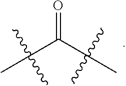 [SMILES: *C(*)=O]

In some variations of formula (25) described in the paragraphs above in which o₂ is 4, Rₐ₁ is H, R_{b1} is CH₃, Rₐ₂ is H, R_{b2} is CH₃, Rₐ₃ and R_{b3} are

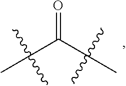 [SMILES: *C(*)=O]

and Rₐ₄ and R_{b4} are

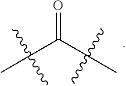 [SMILES: *C(*)=O]

In some variations of formula (25) described in the paragraphs above in which o₂ is 4, Rₐ₁ and R_{b1} are H, Rₐ₂ and R_{b2} are

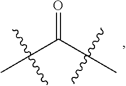 [SMILES: *C(*)=O]

Rₐ₃ and R_{b3} are

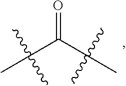 [SMILES: *C(*)=O]

and Rₐ₄ and R_{b4} are

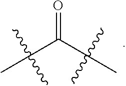 [SMILES: *C(*)=O]

In some variations of formula (25) described in the paragraphs above in which o₂ is 4, Rₐ₁ and R_{b1} are CH₃, Rₐ₂ and R_{b2} are

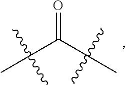 [SMILES: *C(*)=O]

Rₐ₃ and R_{b3} are

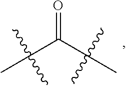 [SMILES: *C(*)=O]

and Rₐ₄ and R_{b4} are

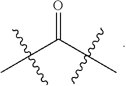 [SMILES: *C(*)=O]

In some variations of formula (25) described in the paragraphs above in which o₂ is 4, Rₐ₁ is H, R_{b1} is CH₃, Rₐ₂ and R_{b2} are

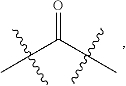 [SMILES: *C(*)=O]

Rₐ₃ and R_{b3} are

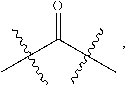 [SMILES: *C(*)=O]

and Rₐ₄ and R_{b4} are

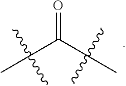 [SMILES: *C(*)=O]

In some variations of formula (25) described in the paragraphs above in which o₂ is 4, Rₐ₁ and R_{b1} are

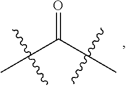 [SMILES: *C(*)=O]

Rₐ₂ and R_{b2} are

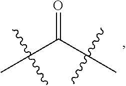 [SMILES: *C(*)=O]

Rₐ₃ and R_{b3} are

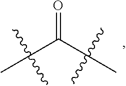 [SMILES: *C(*)=O]

and Rₐ₄ and R_{b4} are

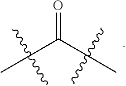 [SMILES: *C(*)=O]

In some variations of formula (25) described in the paragraphs above in which o₂ is 4, Rₐ₁ and R_{b1} are

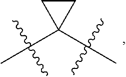 [SMILES: *C1(*)CC1]

Rₐ₂ and R_{b2} are H, Rₐ₃ and R_{b3} are H, and Rₐ₄ and R_{b4} are

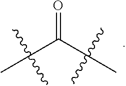 [SMILES: *C(*)=O]

In some variations of formula (25) described in the paragraphs above in which o₂ is 4, Rₐ₁ and R_{b1} are

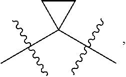 [SMILES: *C1(*)CC1]

Rₐ₂ and R_{b2} are CH₃, Rₐ₃ and R_{b3} are CH₃, and Rₐ₄ and R_{b4} are

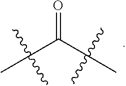 [SMILES: *C(*)=O]

In some variations of formula (25) described in the paragraphs above in which o₂ is 4, Rₐ₁ and R_{b1} are

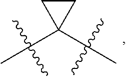 [SMILES: *C1(*)CC1]

Rₐ₂ and R_{b2} are H, Rₐ₃ and R_{b3} are CH₃, and Rₐ₄ and R_{b4} are

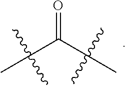 [SMILES: *C(*)=O]

In some variations of formula (25) described in the paragraphs above in which o₂ is 4, Rₐ₁ and R_{b1} are

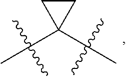 [SMILES: *C1(*)CC1]

Rₐ₂ and R_{b2} are

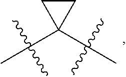 [SMILES: *C1(*)CC1]

Rₐ₃ and R_{b3} are H, and Rₐ₄ and R_{b4} are

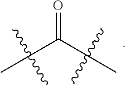 [SMILES: *C(*)=O]

In some variations of formula (25) described in the paragraphs above in which o₂ is 4, Rₐ₁ and R_{b1} are

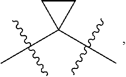 [SMILES: *C1(*)CC1]

Rₐ₂ and R_{b2} are

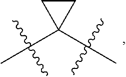 [SMILES: *C1(*)CC1]

Rₐ₃ and R_{b3} are CH₃, and Rₐ₄ and R_{b4} are

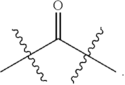 [SMILES: *C(*)=O]

In some variations of formula (25) described in the paragraphs above in which o₂ is 4, Rₐ₁ and R_{b1} are

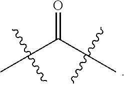 [SMILES: *C(*)=O]

Rₐ₂ and R_{b2} are

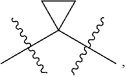 [SMILES: *C1(*)CC1]

Rₐ₃ and R_{b3} are CH₃, and Rₐ₄ and R_{b4} are

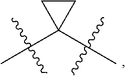 [SMILES: *C1(*)CC1]

In some variations of formula (25) described in the paragraphs above in which o₂ is 4, Rₐ₁ and R_{b1} are

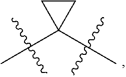 [SMILES: *C1(*)CC1]

Rₐ₂ and R_{b2} are

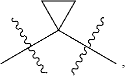 [SMILES: *C1(*)CC1]

Rₐ₃ and R_{b3} are H, and Rₐ₄ and R_{b4} are

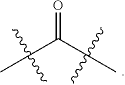 [SMILES: *C(*)=O]

In some variations of formula (25) described in the paragraphs above in which o₂ is 4, Rₐ₁ and R_{b1} are

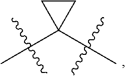 [SMILES: *C1(*)CC1]

Rₐ₂ and R_{b2} are

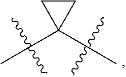 [SMILES: *C1(*)CC1]

Rₐ₃ and R_{b3} are

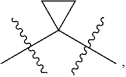 [SMILES: *C1(*)CC1]

and Rₐ₄ and R_{b4} are

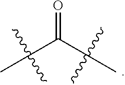 [SMILES: *C(*)=O]

In some variations of formula (25) described in the paragraphs above in which o₂ is 4, Rₐ₁ and R_{b1} are

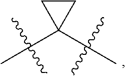 [SMILES: *C1(*)CC1]

Rₐ₂ and R_{b2} are

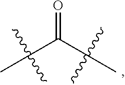 [SMILES: *C(*)=O]

Rₐ₃ and R_{b3} are H, and Rₐ₄ and R_{b4} are

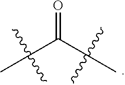 [SMILES: *C(*)=O]

In some variations of formula (25) described in the paragraphs above in which o₂ is 4, Rₐ₁ and R_{b1} are

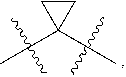 [SMILES: *C1(*)CC1]

Rₐ₂ and R_{b2} are

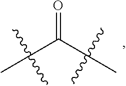 [SMILES: *C(*)=O]

Rₐ₃ and R_{b3} are CH₃, and Rₐ₄ and R_{b4} are

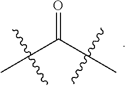 [SMILES: *C(*)=O]

In some variations of formula (25) described in the paragraphs above in which o₂ is 4, Rₐ₁ and R_{b1} are

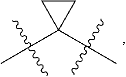 [SMILES: *C1(*)CC1]

Rₐ₂ and R_{b2} are

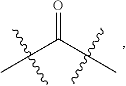 [SMILES: *C(*)=O]

Rₐ₃ and R_{b3} are CH₃, and Rₐ₄ and R_{b4} are

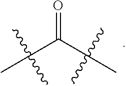 [SMILES: *C(*)=O]

In some variations of formula (25) described in the paragraphs above in which o₂ is 4, Rₐ₁ and R_{b1} are

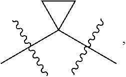 [SMILES: *C1(*)CC1]

Rₐ₂ and R_{b2} are

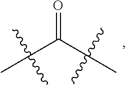 [SMILES: *C(*)=O]

Rₐ₃ and R_{b3} are H, and Rₐ₄ and R_{b4} are

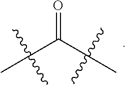 [SMILES: *C(*)=O]

In some variations of formula (25) described in the paragraphs above in which o₂ is 4, Rₐ₁ and R_{b1} are

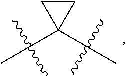 [SMILES: *C1(*)CC1]

Rₐ₂ and R_{b2} are

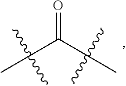 [SMILES: *C(*)=O]

Rₐ₃ and R_{b3} are

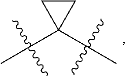 [SMILES: *C1(*)CC1]

and Rₐ₄ and R_{b4} are

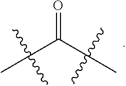 [SMILES: *C(*)=O]

In some variations of formula (25) described in the paragraphs above in which o₂ is 4, Rₐ₁ and R_{b1} are

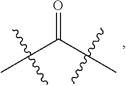 [SMILES: *C(*)=O]

Rₐ₂ and R_{b2} are

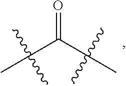 [SMILES: *C(*)=O]

Rₐ₃ and R_{b3} are H, and Rₐ₄ and R_{b4} are

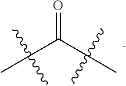 [SMILES: *C(*)=O]

In some variations of formula (25) described in the paragraphs above in which o₂ is 4, Rₐ₁ and R_{b1} are

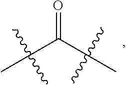 [SMILES: *C(*)=O]

Rₐ₂ and R_{b2} are

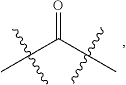 [SMILES: *C(*)=O]

Rₐ₃ and R_{b3} are CH₃, and Rₐ₄ and R_{b4} are

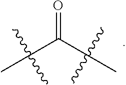 [SMILES: *C(*)=O]

In some variations of formula (25) described in the paragraphs above in which o₂ is 4, Rₐ₁ and R_{b1} are

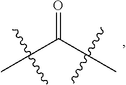 [SMILES: *C(*)=O]

Rₐ₂ and R_{b2} are

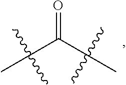 [SMILES: *C(*)=O]

Rₐ₃ and R_{b3} are CH₃, and Rₐ₄ and R_{b4} are

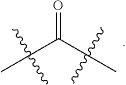 [SMILES: *C(*)=O]

In some variations of formula (25) described in the paragraphs above in which o₂ is 4, Rₐ₁ and R_{b1} are

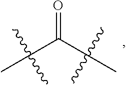 [SMILES: *C(*)=O]

Rₐ₂ and R_{b2} are

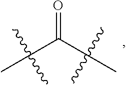 [SMILES: *C(*)=O]

Rₐ₃ and R_{b3} are H, and Rₐ₄ and R_{b4} are

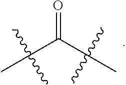 [SMILES: *C(*)=O]

In some variations of formula (25) described in the paragraphs above in which o₂ is 4, Rₐ₁ and R_{b1} and

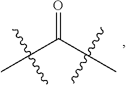 [SMILES: *C(*)=O]

Rₐ₂ and R_{b2} are

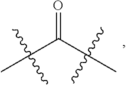 [SMILES: *C(*)=O]

Rₐ₃ and R_{b3} are

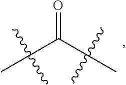 [SMILES: *C(*)=O]

and Rₐ₄ and R_{b4} are

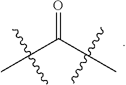 [SMILES: *C(*)=O]

In some variations of formula (25) described in the paragraphs above in which o₂ is 4, Rₐ₁ and R_{b1} are H, Rₐ₂ and R_{b2} are H, and Rₐ₃ and R_{b3} and Rₐ₄ and R_{b4} together are

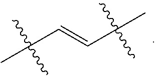 [SMILES: */C=C/*]

In some variations of formula (25) described in the paragraphs above in which O₂ is 4, Rₐ₁ and R_{b1} are H, Rₐ₂ and R_{b2} are CH₃, and Rₐ₃ and R_{b3} and Rₐ₄ and R_{b4} together are

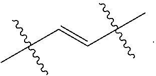 [SMILES: */C=C/*]

In some variations of formula (25) described in the paragraphs above in which o₂ is 4, Rₐ₁ and Rₐ₂ are H, R_{b1} and R_{b2} are CH₃, and Rₐ₃ and R_{b3} and Rₐ₄ and R_{b4} together are

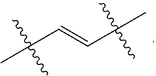 [SMILES: */C=C/*]

In some variations of formula (25) described in the paragraphs above in which o₂ is 4, Rₐ₁ and R_{b1} are H, Rₐ₂ and R_{b2} are

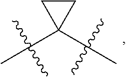 [SMILES: *C1(*)CC1]

and Rₐ₃ and R_{b3} and Rₐ₄ and R_{b4} together are

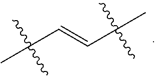 [SMILES: */C=C/*]

In some variations of formula (25) described in the paragraphs above in which o₂ is 4, Rₐ₁ and R_{b1} are

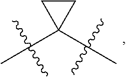 [SMILES: *C1(*)CC1]

Rₐ₂ and R_{b2} are CH₃, and Rₐ₃ and R_{b3} and Rₐ₄ and R_{b4} together are

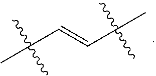 [SMILES: */C=C/*]

In some variations of formula (25) described in the paragraphs above in which o₂ is 4, Rₐ₁ is H, R_{b1} is CH₃, Rₐ₂ and R_{b2} are

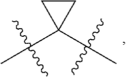 [SMILES: *C1(*)CC1]

and Rₐ₃ and R_{b3} and Rₐ₄ and R_{b4} together are

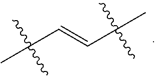 [SMILES: */C=C/*]

In some variations of formula (25) described in the paragraphs above in which o₂ is 4, Rₐ₁ and R_{b1} are H, Rₐ₂ and R_{b2} are

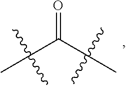 [SMILES: *C(*)=O]

and Rₐ₃ and R_{b3} and Rₐ₄ and R_{b4} together are

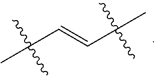 [SMILES: */C=C/*]

In some variations of formula (25) described in the paragraphs above in which o₂ is 4, Rₐ₁ and R_{b1} are

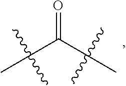 [SMILES: *C(*)=O]

Rₐ₂ and R_{b2} are CH₃, and Rₐ₃ and R_{b3} and Rₐ₄ and R_{b4} together are

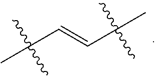 [SMILES: */C=C/*]

In some variations of formula (25) described in the paragraphs above in which o₂ is 4, Rₐ₁ is H, R_{b1} is CH₃, Rₐ₂ and R_{b2} are

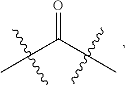 [SMILES: *C(*)=O]

and Rₐ₃ and R_{b3} and Rₐ₄ and R_{b4} together are

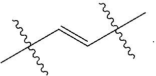 [SMILES: */C=C/*]

In some variations of formula (25) described in the paragraphs above in which o₂ is 4, Rₐ₁ and R_{b1} are

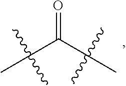 [SMILES: *C(*)=O]

Rₐ₂ and R_{b2} are

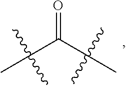 [SMILES: *C(*)=O]

and Rₐ₃ and R_{b3} and Rₐ₄ and R_{b4} together are

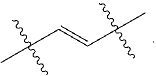 [SMILES: */C=C/*]

In some variations of formula (25) described in the paragraphs above in which o₂ is 4, Rₐ₁ and R_{b1} are

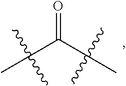 [SMILES: *C(*)=O]

Rₐ₂ and R_{b2} are

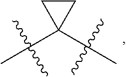 [SMILES: *C1(*)CC1]

and Rₐ₃ and R_{b3} and Rₐ₄ and R_{b4} together are

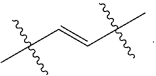 [SMILES: */C=C/*]

In some variations of formula (25) described in the paragraphs above in which o₂ is 4, Rₐ₁ and R_{b1} are

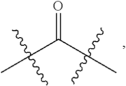 [SMILES: *C(*)=O]

Rₐ₂ and R_{b2} are

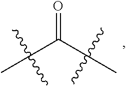 [SMILES: *C(*)=O]

and Rₐ₃ and R_{b3} and Rₐ₄ and R_{b4} together are

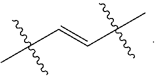 [SMILES: */C=C/*]

In some variations of formula (25) described in the paragraphs above in which o₂ is 4, Rₐ₁ and R_{b1} are

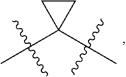 [SMILES: *C1(*)CC1]

Rₐ₂ and R_{b2} are

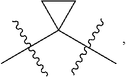 [SMILES: *C1(*)CC1]

and Rₐ₃ and R_{b3} and Rₐ₄ and R_{b4} together are

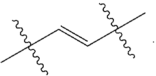 [SMILES: */C=C/*]

In some variations of formula (25) described in the paragraphs above in which o₂ is 4, Rₐ₁ and R_{b1} and Rₐ₂ and R_{b2} together are

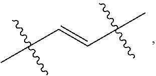 [SMILES: */C=C/*]

and Rₐ₃ and R_{b3} and Rₐ₄ and R_{b4} together are

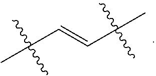 [SMILES: */C=C/*]

In some variations of formula (25) described in the paragraphs above, ring A is

ring A is

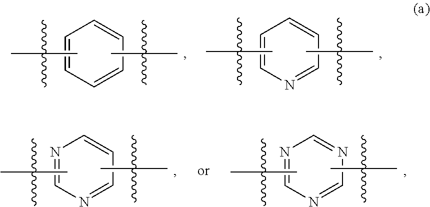

optionally substituted with halo or C1-C6 linear or branched alkyl;

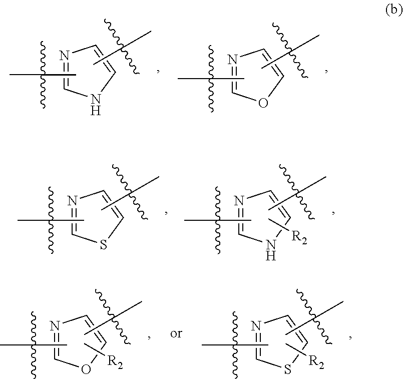

optionally substituted with halo or C1-C6 linear or branched alkyl; or

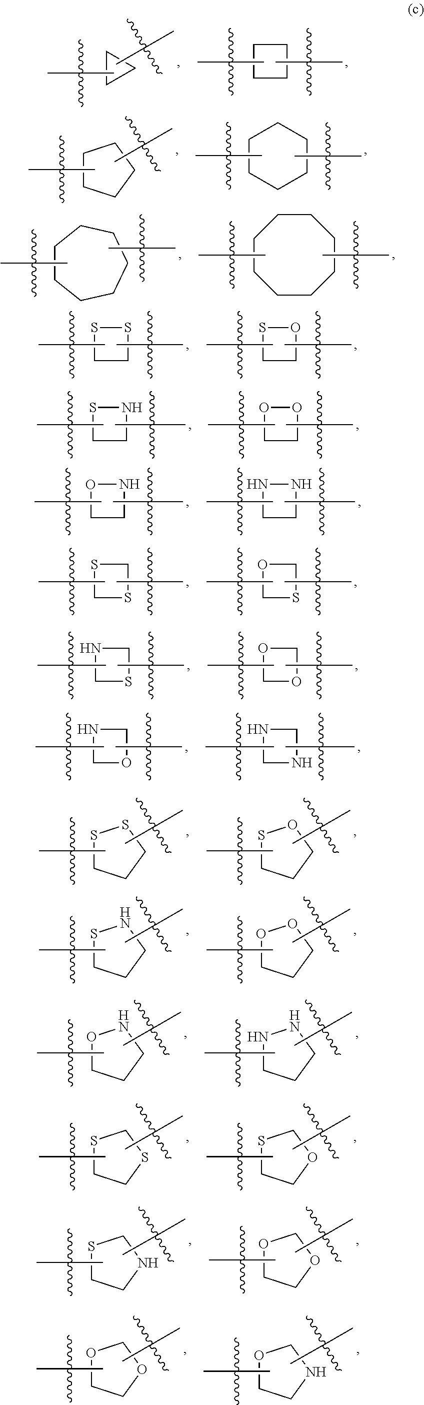

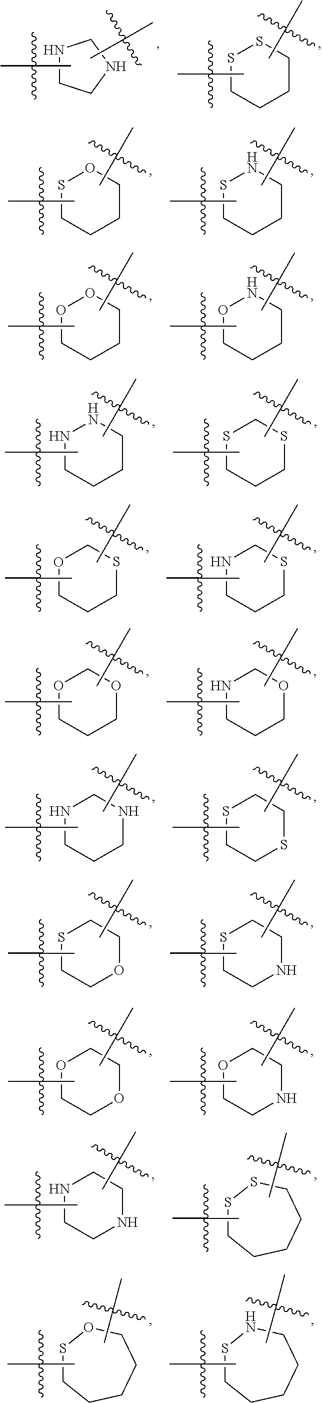

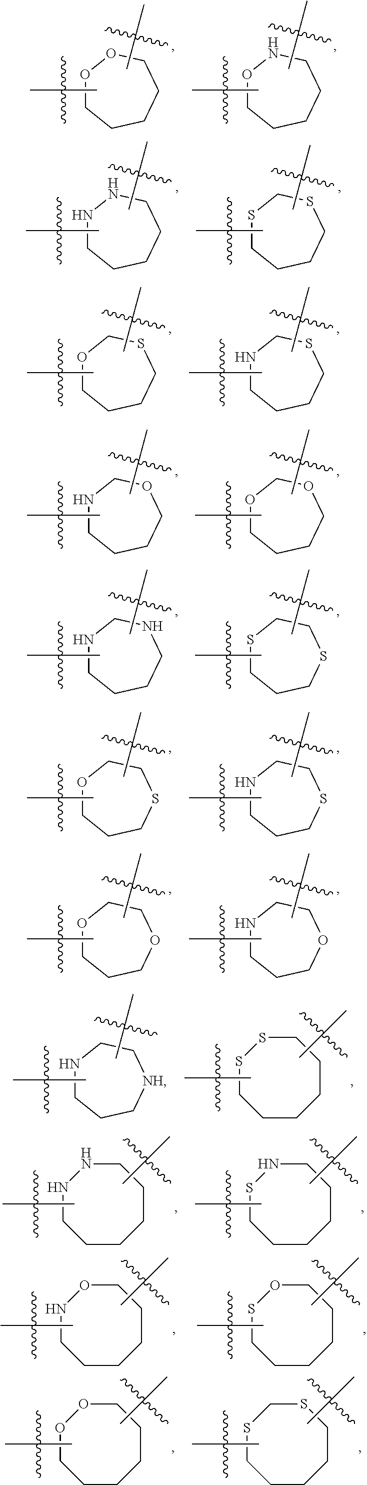

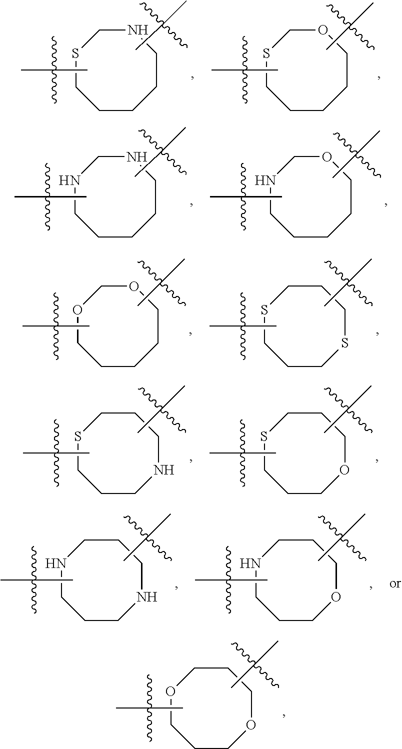

optionally substituted with halo or C1-C6 linear or branched alkyl.

In some variations of formula (25) described in the paragraphs above, n is 1-4, 1-3, 1-2, 2-4, 2-3, 3-4, 1, 2, 3, or 4.

In some variations of formula (25) described in the paragraphs above, ring A is substituted with halo. In some variations, the halo is F, Br, I, or Cl.

In some variations of formula (25) described in the paragraphs above, ring A is substituted with C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (25) described in the paragraphs above, D1 is safranin-O. In some variations of formula (25) described in the paragraphs above, the first D2 is safranin-O. In some variations of formula (25) described in the paragraphs above, the second D2 is safranin-O. In some variations of formula (25) described in the paragraphs above, D1 and the first D2 are safranin-O. In some variations of formula (25) described in the paragraphs above, D1 and the second D2 are safranin-O. In some variations of formula (25) described in the paragraphs above, the first D2 and the second D2 are safranin-O.

In some variations of formula (25) described in the paragraph above, the pendant phenyl ring of D1 is unsubstituted. In some variations of formula (25) described in the paragraph above, the pendant phenyl ring of D1 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D1 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D1 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (25) described in the two paragraphs above, the pendant phenyl ring of the first D2 is unsubstituted. In some variations of formula (25) described in the paragraph above, the pendant phenyl ring of the first D2 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of the first D2 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of the first D2 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (25) described in the three paragraphs above, the pendant phenyl ring of the second D2 is unsubstituted. In some variations of formula (25) described in the paragraph above, the pendant phenyl ring of the second D2 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of the second D2 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of the second D2 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some of the variations of formula (25) described above D1 and D2 are safranin-O moieties, as shown in formula (25a):

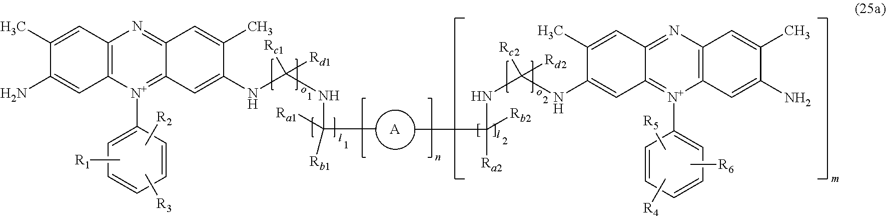

in which m, n, l₁, l₂, o₁, o₂, ring A, Rₐ₁, R_{b1}, Rₐ₂, R_{b2} R_{c2}, R_{d1}, R_{d2}, and R_{d2} are as defined in the paragraphs above, R₁, R₂, R₃, R₄, R₅, and R₆ independently are absent or independently are selected from —NH₂, —NHR, —NR₂, —OH, —O⁻, —NHCOCH₃, —NHCOR, —OCH₃, —OR, —C₂H₅, —R, —C₆H₅, —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR), and R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

One of skill in the art can readily visualize and prepare other cationic multimers falling within formula (25) in which other cationic dye moieties are used in place of one or both of the safranin-O moieties.

Some cationic dye multimers fall within formula (26), (27), (28), (29), (30), (31), or (32):

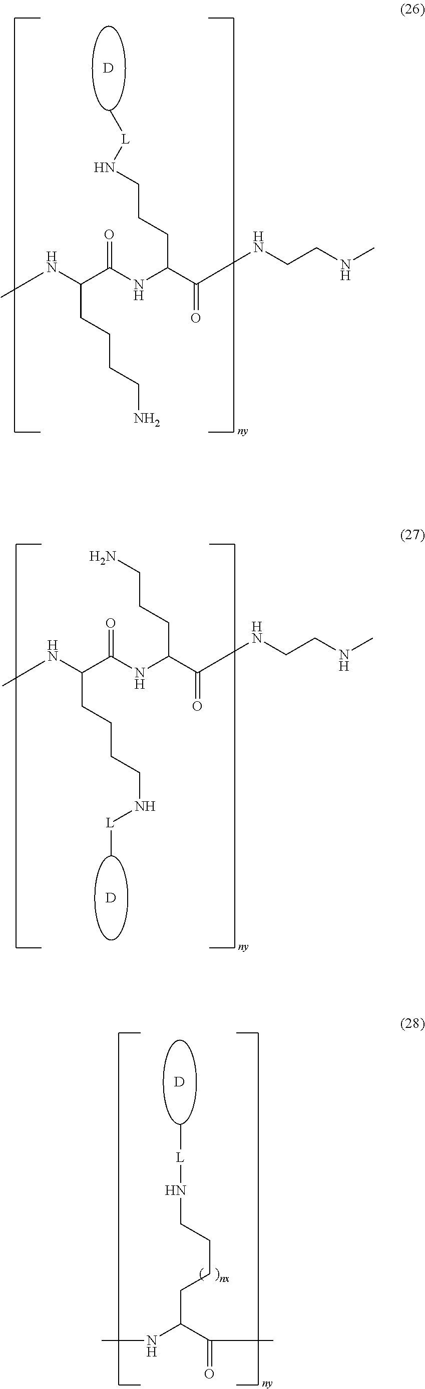

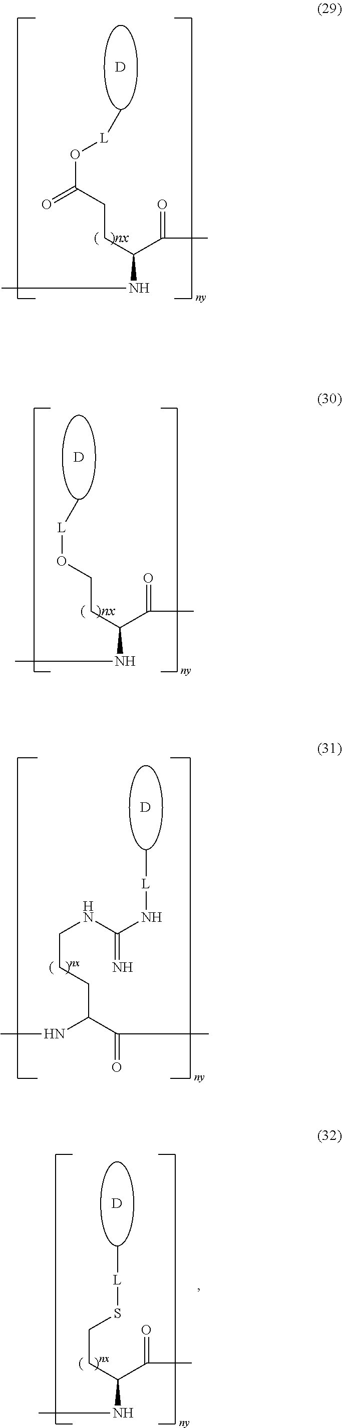

wherein D is a cationic dye moiety, nx is 0-5, ny is 1-5, and L is absent or L is a linker selected from linker (a.1), linker (a.2), linker (b.1), linker (c.1), linker (c.2), linker (d), linker (e.1), linker (f.1), linker (f.2), linker (g.1), linker (g.2), linker (h.1), linker (h.2), linker (i.1), linker (i.2), linker (j.1), linker (j.2), linker (k), linker (l.1), linker (l.2), linker (m.1), linker (n.1), linker (n.2), linker (o), linker (p), linker (q), linker (r), and linker (s), described above.

In some variations of formula (26), (27), (28), (29), (30), (31), or (32), L is linker (a.1):

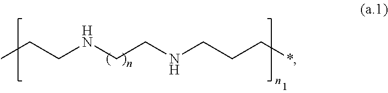

in which n is 1-6, n₁ is 1-4, and * is the attachment site for the cationic dye moiety D.

In some variations of formula (26), (27), (28), (29), (30), (31), or (32) in which L is linker (a.1), n is 1-6, 1-5, 1-4, 1-3, 1-2, 2-6, 2-5, 2-4, 2-3, 3-6, 3-5, 3-4, 4-6, 4-5, 5-6, 1, 2, 3, 4, 5, or 6.

In some variations of formula (26), (27), (28), (29), (30), (31), or (32) described in the paragraphs above in which L is linker (a.1), n₁ is 1-4, 1-3, 1-2, 2-4, 2-3, 3-4, 1, 2, 3, or 4.

In some variations of formula (26), (27), (28), (29), (30), (31), or (32), L is linker (a.2):

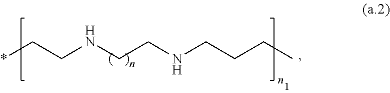

in which n is 1-6, n₁ is 1-4, and * is the attachment site for the cationic dye moiety D.

In some variations of formula (26), (27), (28), (29), (30), (31), or (32) in which L is linker (a.2), n is 1-6, 1-5, 1-4, 1-3, 1-2, 2-6, 2-5, 2-4, 2-3, 3-6, 3-5, 3-4, 4-6, 4-5, 5-6, 1, 2, 3, 4, 5, or 6.

In some variations of formula (26), (27), (28), (29), (30), (31), or (32) described in the paragraphs above in which L is linker (a.2), n₁ is 1-4, 1-3, 1-2, 2-4, 2-3, 3-4, 1, 2, 3, or 4.

In some variations of formula (26), (27), (28), (29), (30), (31), or (32), L is linker (b.1):

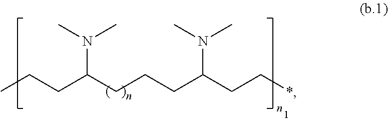

in which n is 0-6, n₁ is 1-4, and * is the attachment site for the cationic dye moiety D.

In some variations of formula (26), (27), (28), (29), (30), (31), or (32) in which L is linker (b.1), n is 0-6, 0-5, 0-4, 0-3, 0-2, 0-1, 1-6, 1-5, 1-4, 1-3, 1-2, 2-6, 2-5, 2-4, 2-3, 3-6, 3-5, 3-4, 4-6, 4-5, 5-6, 0, 1, 2, 3, 4, 5, or 6.

In some variations of formula (26), (27), (28), (29), (30), (31), or (32) described in the paragraphs above in which L is linker (b.1), n₁ is 1-4, 1-3, 1-2, 2-4, 2-3, 3-4, 1, 2, 3, or 4.

In some variations of formula (26), (27), (28), (29), (30), (31), or (32), L is linker (c.1):

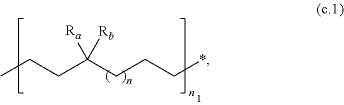

in which n is 0-6, n₁ is 1-4, either (1) Rₐ and R_{b} independently are H or CH₃ or (2) Rₐ and R_{b} are

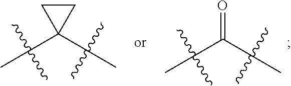

or (3) two of CRₐR_{b} are

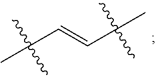 [SMILES: */C=C/*]

and * is the attachment site for the cationic dye moiety D.

In some variations of formula (26), (27), (28), (29), (30), (31), or (32) in which L is linker (c.1), n is 0-6, 0-5, 0-4, 0-3, 0-2, 0-1, 1-6, 1-5, 1-4, 1-3, 1-2, 2-6, 2-5, 2-4, 2-3, 3-6, 3-5, 3-4, 4-6, 4-5, 5-6, 0, 1, 2, 3, 4, 5, or 6.

In some variations of formula (26), (27), (28), (29), (30), (31), or (32) described in the paragraphs above in which L is linker (c.1), n₁ is 1-4, 1-3, 1-2, 2-4, 2-3, 3-4, 1, 2, 3, or 4.

In some variations of formula (26), (27), (28), (29), (30), (31), or (32) described in the paragraphs above in which L is linker (c.1), Rₐ is H and R_{b} is H. In some variations of formula (26), (27), (28), (29), (30), (31), or (32) described in the paragraphs above in which L is linker (c.1), Rₐ is H and R_{b} is CH₃. In some variations of formula (26), (27), (28), (29), (30), (31), or (32) described in the paragraphs above in which L is linker (c.1), Rₐ and R_{b} are

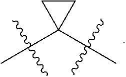 [SMILES: *C1(*)CC1]

In some variations of formula (26), (27), (28), (29), (30), (31), or (32) described in the paragraphs above in which L is linker (c.1), Rₐ and R_{b} are

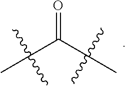 [SMILES: *C(*)=O]

In some variations of formula (26), (27), (28), (29), (30), (31), or (32) described in the paragraphs above in which L is linker (c.1), two of CRₐR_{b} are

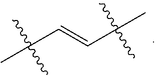 [SMILES: */C=C/*]

In some variations of formula (26), (27), (28), (29), (30), (31), or (32), L is linker (c.2):

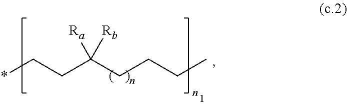

in which n is 0-6, n₁ is 1-4, either (1) Rₐ and R_{b} independently are H or CH₃ or (2) Rₐ and R_{b} are

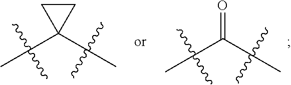

or (3) two of CRₐR_{b} are

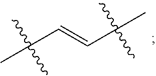 [SMILES: */C=C/*]

and * is the attachment site for the cationic dye moiety D.

In some variations of formula (26), (27), (28), (29), (30), (31), or (32) in which L is linker (c.2), n is 0-6, 0-5, 0-4, 0-3, 0-2, 0-1, 1-6, 1-5, 1-4, 1-3, 1-2, 2-6, 2-5, 2-4, 2-3, 3-6, 3-5, 3-4, 4-6, 4-5, 5-6, 0, 1, 2, 3, 4, 5, or 6.

In some variations of formula (26), (27), (28), (29), (30), (31), or (32) described in the paragraphs above in which L is linker (c.2), n₁ is 1-4, 1-3, 1-2, 2-4, 2-3, 3-4, 1, 2, 3, or 4.

In some variations of formula (26), (27), (28), (29), (30), (31), or (32) described in the paragraphs above in which L is linker (c.2), Rₐ is H and R_{b} is H. In some variations of formula (26), (27), (28), (29), (30), (31), or (32) described in the paragraphs above in which L is linker (c.2), Rₐ is H and R_{b} is CH₃. In some variations of formula (26), (27), (28), (29), (30), (31), or (32) described in the paragraphs above in which L is linker (c.2), Rₐ and R_{b} are

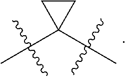 [SMILES: *C1(*)CC1]

In some variations of formula (26), (27), (28), (29), (30), (31), or (32) described in the paragraphs above in which L is linker (c.2), Rₐ and R_{b} are

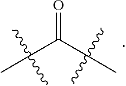 [SMILES: *C(*)=O]

In some variations of formula (26), (27), (28), (29), (30), (31), or (32) described in the paragraphs above in which L is linker (c.2), two of CRₐR_{b} are

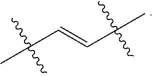 [SMILES: */C=C/*]

In some variations of formula (26), (27), (28), (29), (30), (31), or (32) described in the paragraphs above in which L is linker (c), L is

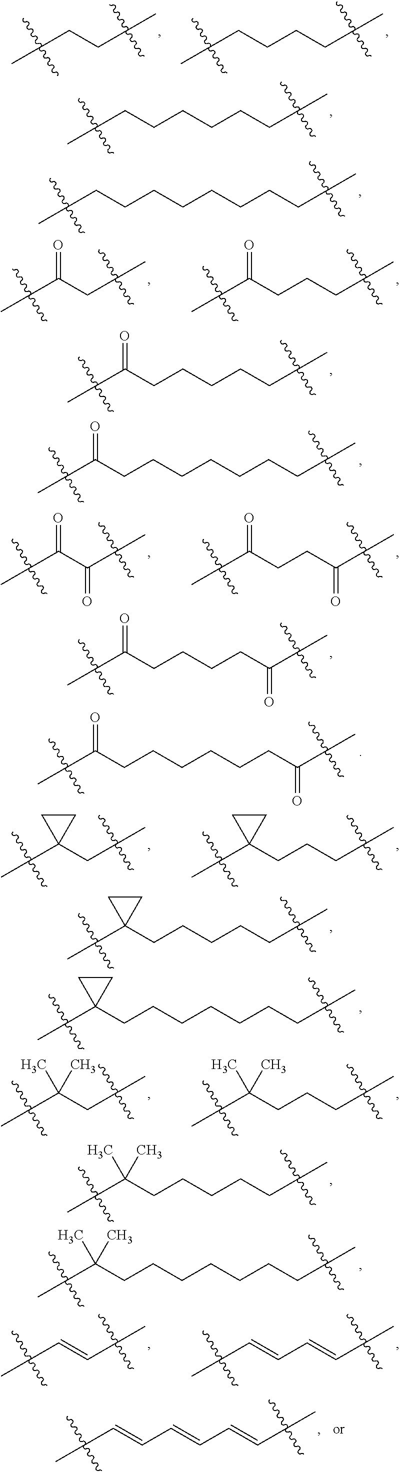

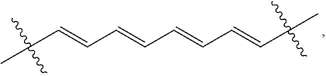 [SMILES: */C=C/C=C/C=C/C=C/*]

In some variations of formula (26), (27), (28), (29), (30), (31), or (32), L is linker (d):

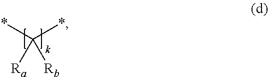

where k is 2-10; (1) Rₐ and R_{b} independently are H or CH₃, or (2) Rₐ and R_{b} are

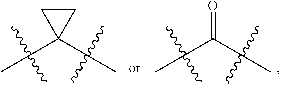

or (3) two of CRₐR_{b} are

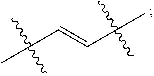 [SMILES: */C=C/*]

and * is the attachment site for the cationic dye moiety D

In some variations of formula (26), (27), (28), (29), (30), (31), or (32) in which L is linker (d), k is 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, 9-10, 2, 3, 4, 5, 6, 7, 8, 9, or 10.

In some variations of formula (26), (27), (28), (29), (30), (31), or (32) described in the paragraphs above in which L is linker (d), Rₐ is H and R_{b} is H. In some variations of formula (26), (27), (28), (29), (30), (31), or (32) described in the paragraphs above in which L is linker (d), Rₐ is H and R_{b} is CH₃. In some variations of formula (26), (27), (28), (29), (30), (31), or (32) described in the paragraphs above in which L is linker (d), Rₐ and R_{b} are

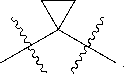 [SMILES: *C1(*)CC1]

In some variations of formula (26), (27), (28), (29), (30), (31), or (32) described in the paragraphs above in which L is linker (d), Rₐ and R_{b} are

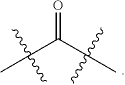 [SMILES: *C(*)=O]

In some variations of formula (26), (27), (28), (29), (30), (31), or (32) described in the paragraphs above in which L is linker (d), two of CRₐR_{b} are

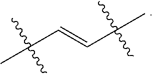 [SMILES: */C=C/*]

In some variations of formula (26), (27), (28), (29), (30), (31), or (32), L is linker (e.1):

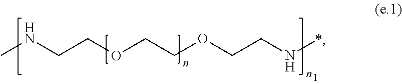

in which n is 0-6, n₁ is 1-4, and * is the attachment site for the cationic dye moiety D.

In some variations of formula (26), (27), (28), (29), (30), (31), or (32) in which L is linker (e.1), n is 0-6, 0-5, 0-4, 0-3, 0-2, 0-1, 1-6, 1-5, 1-4, 1-3, 1-2, 2-6, 2-5, 2-4, 2-3, 3-6, 3-5, 3-4, 4-6, 4-5, 5-6, 0, 1, 2, 3, 4, 5, or 6.

In some variations of formula (26), (27), (28), (29), (30), (31), or (32) described in the paragraphs above in which L is linker (e.1), n₁ is 1-4, 1-3, 1-2, 2-4, 2-3, 3-4, 1, 2, 3, or 4.

In some variations of formula (26), (27), (28), (29), (30), (31), or (32), L is linker (f.1):

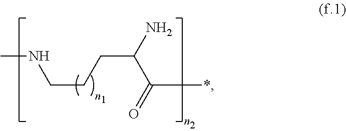

in which n₁ is 0-5, n₂ is 1-5, and * is the attachment site for the cationic dye moiety D.

In some variations of formula (26), (27), (28), (29), (30), (31), or (32) in which L is linker (f.1), n₁ is 0-5, 0-4, 0-3, 0-2, 0-1, 1-5, 1-4, 1-3, 1-2, 2-5, 2-4, 2-3, 3-5, 3-4, 4-5, 0, 1, 2, 3, 4, or 5.

In some variations of formula (26), (27), (28), (29), (30), (31), or (32) described in the paragraphs above in which L is linker (f.1), n₂ is 1-5, 1-4, 1-3, 1-2, 2-5, 2-4, 2-3, 3-5, 3-4, 4-5, 1, 2, 3, 4, or 5.

In some variations of formula (26), (27), (28), (29), (30), (31), or (32), L is linker (f.2):

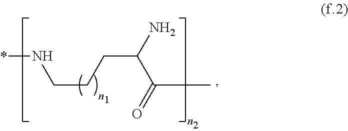

in which n₁ is 0-5, n₂ is 1-5, and * is the attachment site for the cationic dye moiety D.

In some variations of formula (26), (27), (28), (29), (30), (31), or (32) in which L is linker (f.2), n₁ is 0-5, 0-4, 0-3, 0-2, 0-1, 1-5, 1-4, 1-3, 1-2, 2-5, 2-4, 2-3, 3-5, 3-4, 4-5, 0, 1, 2, 3, 4, or 5.

In some variations of formula (26), (27), (28), (29), (30), (31), or (32) described in the paragraphs above in which L is linker (f.2), n₂ is 1-5, 1-4, 1-3, 1-2, 2-5, 2-4, 2-3, 3-5, 3-4, 4-5, 1, 2, 3, 4, or 5.

In some variations of formula (26), (27), (28), (29), (30), (31), or (32), L is linker (g.1):

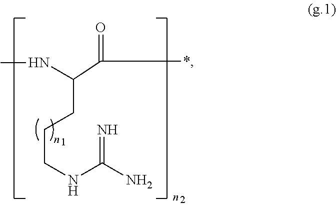

in which n₁ is 0-5, n₂ is 1-5, and * is the attachment site for the cationic dye moiety D.

In some variations of formula (26), (27), (28), (29), (30), (31), or (32) in which L is linker (g.1), n₁ is 0-5, 0-4, 0-3, 0-2, 0-1, 1-5, 1-4, 1-3, 1-2, 2-5, 2-4, 2-3, 3-5, 3-4, 4-5, 0, 1, 2, 3, 4, or 5.

In some variations of formula (26), (27), (28), (29), (30), (31), or (32) described in the paragraphs above in which L is linker (g.1), n₂ is 1-5, 1-4, 1-3, 1-2, 2-5, 2-4, 2-3, 3-5, 3-4, 4-5, 1, 2, 3, 4, or 5.

In some variations of formula (26), (27), (28), (29), (30), (31), or (32), L is linker (g.2):

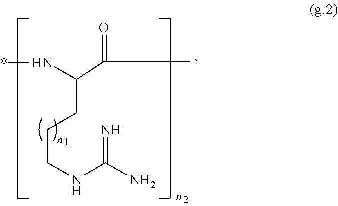

in which n₁ is 0-5, n₂ is 1-5, and * is the attachment site for the cationic dye moiety D.

In some variations of formula (26), (27), (28), (29), (30), (31), or (32) in which L is linker (g.2), n₁ is 0-5, 0-4, 0-3, 0-2, 0-1, 1-5, 1-4, 1-3, 1-2, 2-5, 2-4, 2-3, 3-5, 3-4, 4-5, 0, 1, 2, 3, 4, or 5.

In some variations of formula (26), (27), (28), (29), (30), (31), or (32) described in the paragraphs above in which L is linker (g.2), n₂ is 1-5, 1-4, 1-3, 1-2, 2-5, 2-4, 2-3, 3-5, 3-4, 4-5, 1, 2, 3, 4, or 5.

In some variations of formula (26), (27), (28), (29), (30), (31), or (32), L is linker (h.1):

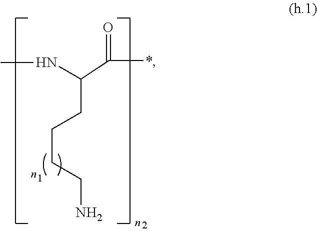

in which n₁ is 0-5, n₂ is 1-5, and * is the attachment site for the cationic dye moiety D.

In some variations of formula (26), (27), (28), (29), (30), (31), or (32) in which L is linker (h.1), n₁ is 0-5, 0-4, 0-3, 0-2, 0-1, 1-5, 1-4, 1-3, 1-2, 2-5, 2-4, 2-3, 3-5, 3-4, 4-5, 0, 1, 2, 3, 4, or 5.

In some variations of formula (26), (27), (28), (29), (30), (31), or (32) described in the paragraphs above in which L is linker (h.1), n₂ is 1-5, 1-4, 1-3, 1-2, 2-5, 2-4, 2-3, 3-5, 3-4, 4-5, 1, 2, 3, 4, or 5.

In some variations of formula (26), (27), (28), (29), (30), (31), or (32), L is linker (h.2):

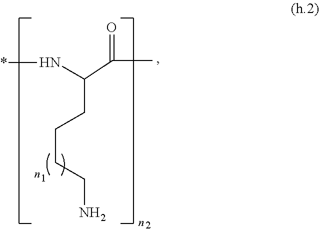

in which n₁ is 0-5, n₂ is 1-5, and * is the attachment site for the cationic dye moiety D.

In some variations of formula (26), (27), (28), (29), (30), (31), or (32) in which L is linker (h.2), n₁ is 0-5, 0-4, 0-3, 0-2, 0-1, 1-5, 1-4, 1-3, 1-2, 2-5, 2-4, 2-3, 3-5, 3-4, 4-5, 0, 1, 2, 3, 4, or 5.

In some variations of formula (26), (27), (28), (29), (30), (31), or (32) described in the paragraphs above in which L is linker (h.2), n₂ is 1-5, 1-4, 1-3, 1-2, 2-5, 2-4, 2-3, 3-5, 3-4, 4-5, 1, 2, 3, 4, or 5.

In some variations of formula (26), (27), (28), (29), (30), (31), or (32), L is linker (i.1):

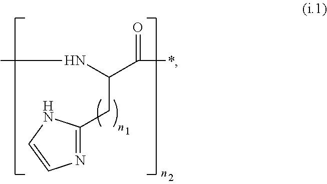

in which n₁ and n₂ independently are 1-5 and * is the attachment site for the cationic dye moiety D.

In some variations of formula (26), (27), (28), (29), (30), (31), or (32) described in the paragraphs above, L is linker (i.1), n₁ is 1-5, 1-4, 1-3, 1-2, 2-5, 2-4, 2-3, 3-5, 3-4, 4-5, 1, 2, 3, 4, or 5.

In some variations of formula (26), (27), (28), (29), (30), (31), or (32) described in the paragraphs above in which L is linker (i.1), n₁ is 1-5, 1-4, 1-3, 1-2, 2-5, 2-4, 2-3, 3-5, 3-4, 4-5, 1, 2, 3, 4, or 5.

In some variations of formula (26), (27), (28), (29), (30), (31), or (32), L is linker (i.2):

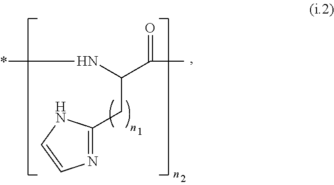

in which n₁ and n₂ independently are 1-5 and * is the attachment site for the cationic dye moiety D.

In some variations of formula (26), (27), (28), (29), (30), (31), or (32) described in the paragraphs above, L is linker (i.2), n₁ is 1-5, 1-4, 1-3, 1-2, 2-5, 2-4, 2-3, 3-5, 3-4, 4-5, 1, 2, 3, 4, or 5.

In some variations of formula (26), (27), (28), (29), (30), (31), or (32) described in the paragraphs above in which L is linker (i.2), n₁ is 1-5, 1-4, 1-3, 1-2, 2-5, 2-4, 2-3, 3-5, 3-4, 4-5, 1, 2, 3, 4, or 5.

In some variations of formula (26), (27), (28), (29), (30), (31), or (32), L is linker (j.1):

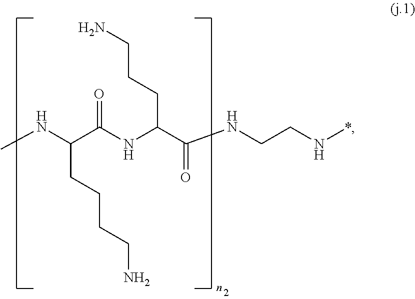

in which n₂ is 1-5 and * is the attachment site for the cationic dye moiety D.

In some variations of formula (26), (27), (28), (29), (30), (31), or (32) in which L is linker (j.1), n₂ is 1-5, 1-4, 1-3, 1-2, 2-5, 2-4, 2-3, 3-5, 3-4, 4-5, 1, 2, 3, 4, or 5.

In some variations of formula (26), (27), (28), (29), (30), (31), or (32), L is linker (j.2):

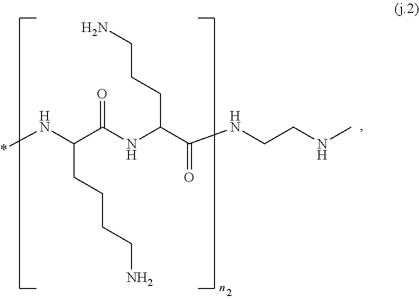

in which n₂ is 1-5 and * is the attachment site for the cationic dye moiety D.

In some variations of formula (26), (27), (28), (29), (30), (31), or (32) in which L is linker (j.2), n₂ is 1-5, 1-4, 1-3, 1-2, 2-5, 2-4, 2-3, 3-5, 3-4, 4-5, 1, 2, 3, 4, or 5.

In some variations of formula (26), (27), (28), (29), (30), (31), or (32), L is linker (k):

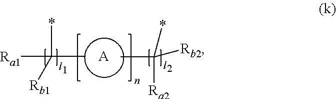

in which l₁ and l₂ independently are 1-4, n is 1-4; ring A is aryl, heteroaryl, cycloalkyl, or heterocyclyl; (1) Rₐ₁ and R_{b1} independently are H or CH₃ or (2) Rₐ₁ and R_{b1} independently are

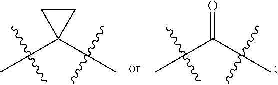

or (3) two of CRₐ₁Rₘ are

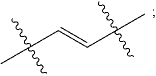 [SMILES: */C=C/*]

(1) Rₐ₂ and R_{b2} independently are H or CH₃ or (2) Rₐ₂ and R_{b2} independently are

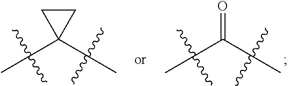

or (3) two of CRₐ₂R_{b2} are

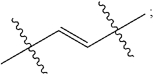 [SMILES: */C=C/*]

and * is the attachment site for the cationic dye moiety D.

In some variations of formula (26), (27), (28), (29), (30), (31), or (32) in which L is linker (k), l₁ is 1-4, 1-3, 1-3, 1-2, 2-4, 2-3, 3-4, 1, 2, 3, or 4.

In some variations of formula (26), (27), (28), (29), (30), (31), or (32) described in the paragraphs above in which L is linker (k), l₂ is 1-4, 1-3, 1-3, 1-2, 2-4, 2-3, 3-4, 1, 2, 3, or 4.

In some variations of formula (26), (27), (28), (29), (30), (31), or (32) described in the paragraphs above in which L is linker (k), Rₐ₁ is H and R_{b1} is H. In some variations of formula (26), (27), (28), (29), (30), (31), or (32) described in the paragraphs above in which L is linker (k), Rₐ₁ is H and R_{b1} is CH₃. In some variations of formula (26), (27), (28), (29), (30), (31), or (32) described in the paragraphs above in which L is linker (k), Rₐ₁ and R_{b1} are

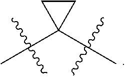 [SMILES: *C1(*)CC1]

In some variations of formula (26), (27), (28), (29), (30), (31), or (32) described in the paragraphs above in which L is linker (k), Rₐ₁ and R_{b1} are

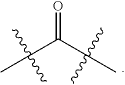 [SMILES: *C(*)=O]

In some variations of formula (26), (27), (28), (29), (30), (31), or (32) described in the paragraphs above in which L is linker (k), two of CRₐ₁R_{b1} are

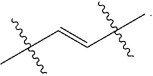 [SMILES: */C=C/*]

In some variations of formula (26), (27), (28), (29), (30), (31), or (32) described in the paragraphs above in which L is linker (k), Rₐ₂ is H and R_{b2} is H. In some variations of formula (26), (27), (28), (29), (30), (31), or (32) described in the paragraphs above in which L is linker (k), Rₐ₂ is H and R_{b2} is CH₃. In some variations of formula (26), (27), (28), (29), (30), (31), or (32) described in the paragraphs above in which L is linker (k), Rₐ₂ and R_{b2} are

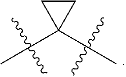 [SMILES: *C1(*)CC1]

In some variations of formula (26), (27), (28), (29), (30), (31), or (32) described in the paragraphs above in which L is linker (k), Rₐ₂ and R_{b2} are

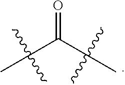 [SMILES: *C(*)=O]

In some variations of formula (26), (27), (28), (29), (30), (31), or (32) described in the paragraphs above in which L is linker (k), two of CRₐ₂R_{b2} are

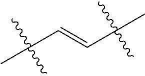 [SMILES: */C=C/*]

In some variations of formula (26), (27), (28), (29), (30), (31), or (32) described in the paragraphs above in which L is linker (k), ring A is

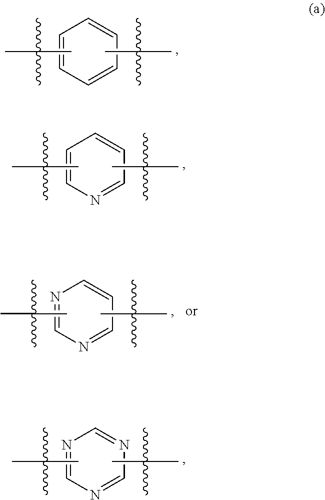

optionally substituted with halo or C1-C6 linear or branched alkyl;

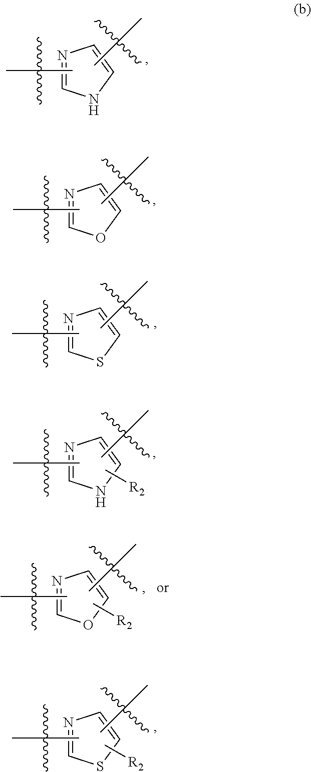

optionally substituted with halo or C1-C6 linear or branched alkyl; or

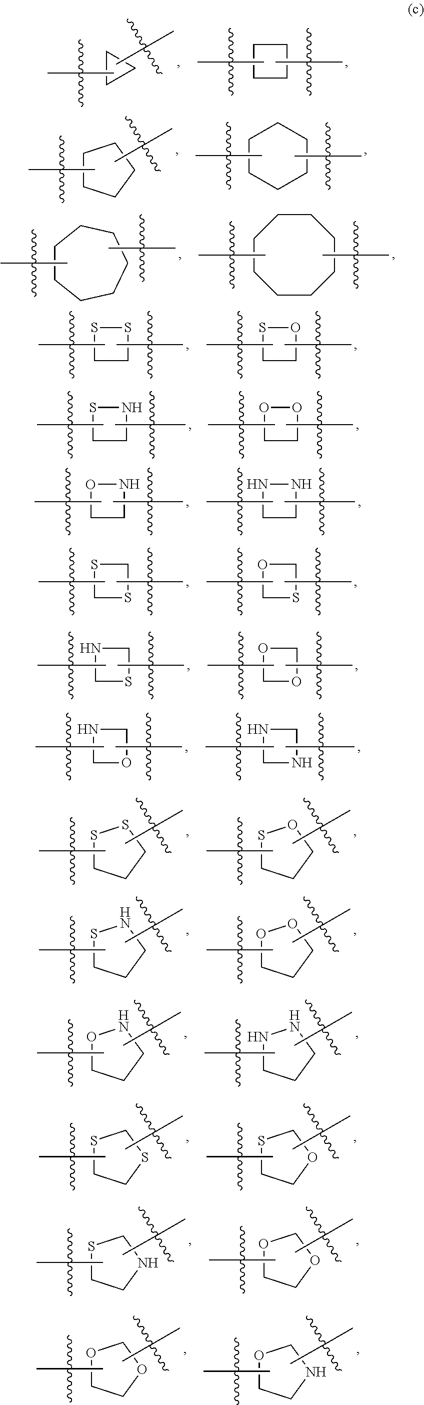

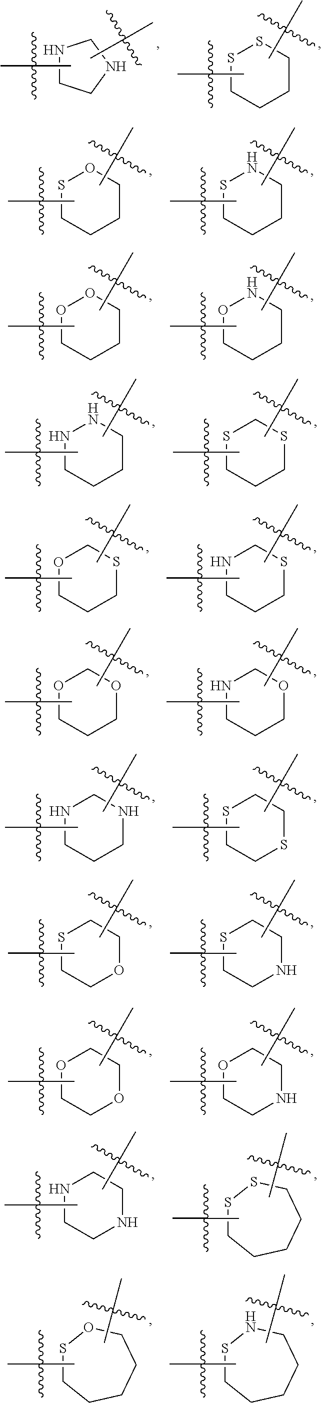

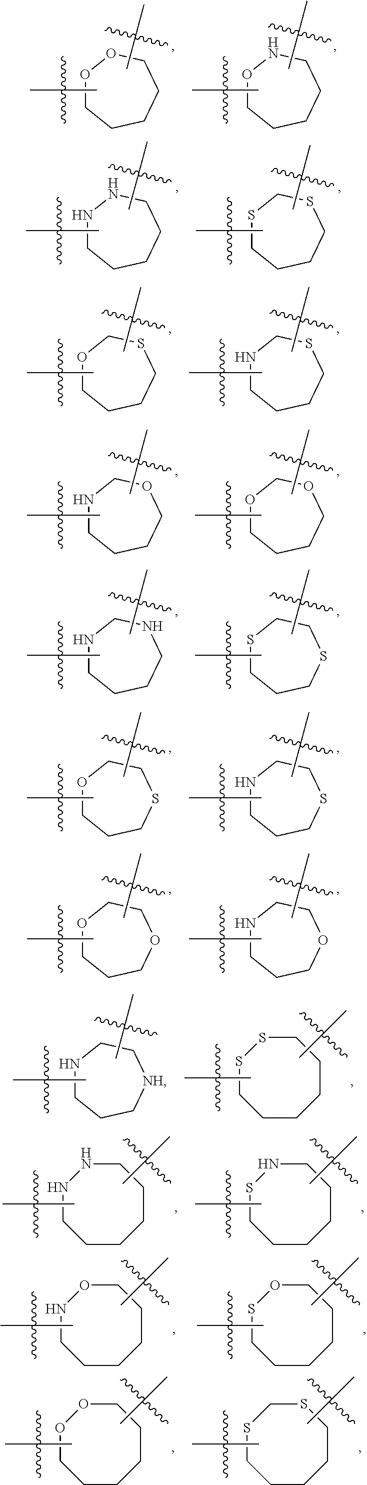

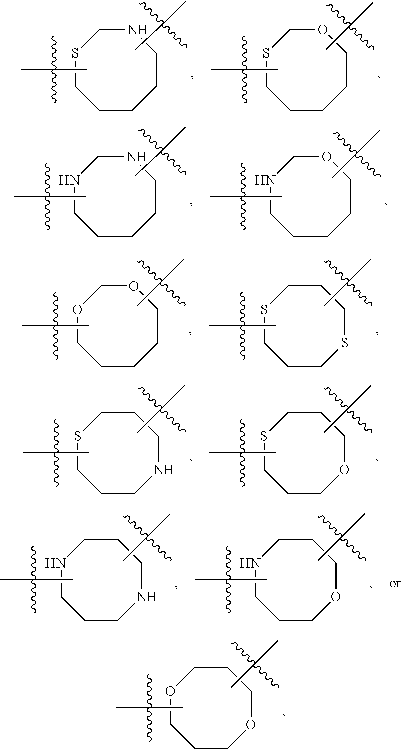

optionally substituted with halo or C1-C6 linear or branched alkyl.

In some variations of formula (26), (27), (28), (29), (30), (31), or (32) described in the paragraphs above in which L is linker (k), ring A is substituted with halo. In some variations, the halo is F, Br, I, or Cl.

In some variations of formula (26), (27), (28), (29), (30), (31), or (32) described in the paragraphs above in which L is linker (k), ring A is substituted with C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (26), (27), (28), (29), (30), (31), or (32), L is linker (l.1):

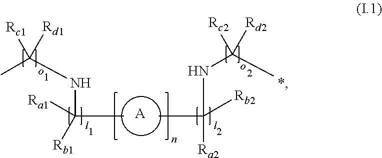

in which l₁, l₂, n, o₁, and o₂ independently are 1-4; ring A is aryl, heteroaryl, cycloalkyl, or heterocyclyl; for each independent instance of Rₐ₁ and R_{b1}, Rₐ₁ and R_{b1} (1) independently are H or CH₃, or (2) Rₐ₁ and R_{b1} independently are

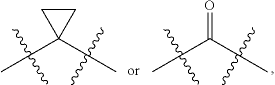

or (3) two of CRₐ₁R_{b1} are

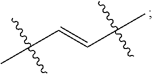 [SMILES: ";" ; */C=C/*]

for each independent instance of Rₐ₂ and R_{b2}, Rₐ₂ and R_{b2} (1) independently are H or CH₃, or (2) Rₐ₂ and R_{b2} independently are

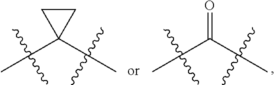

or (3) two of CRₐ₂R_{b2} are

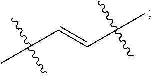 [SMILES: ";" ; */C=C/*]

for each independent instance of R_{c1} and R_{d1}, R_{c1} and R_{d1} (1) independently are H or CH₃, or (2) R_{c1} and R_{d1} independently are

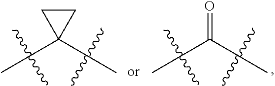

or (3) two of CR_{c1}R_{d1} are

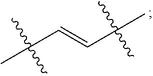 [SMILES: ";" ; */C=C/*]

for each independent instance of R_{c2} and R_{d2}, R_{c2} and R_{d2} (1) independently are H or CH₃, or (2) R_{c2} and R_{d2} independently are

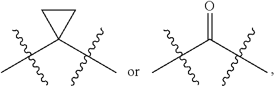

or (3) two of CR_{c2}R_{d2} are

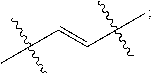 [SMILES: ";" ; */C=C/*]

and * is the attachment site for the cationic dye moiety D.

In some variations of formula (26), (27), (28), (29), (30), (31), or (32) in which L is linker (l.1), l₁ is 1-4, 1-3, 1-3, 1-2, 2-4, 2-3, 3-4, 1, 2, 3, or 4.

In some variations of formula (26), (27), (28), (29), (30), (31), or (32) described in the paragraphs above in which L is linker (l.1), l₂ is 1-4, 1-3, 1-3, 1-2, 2-4, 2-3, 3-4, 1, 2, 3, or 4.

In some variations of formula (26), (27), (28), (29), (30), (31), or (32) described in the paragraphs above in which L is linker (l.1), l₁ is 1-4, 1-3, 1-3, 1-2, 2-4, 2-3, 3-4, 1, 2, 3, or 4.

In some variations of formula (26), (27), (28), (29), (30), (31), or (32) described in the paragraphs above in which L is linker (l.1), o₂ is 1-4, 1-3, 1-3, 1-2, 2-4, 2-3, 3-4, 1, 2, 3, or 4.

In some variations of formula (26), (27), (28), (29), (30), (31), or (32) described in the paragraphs above in which L is linker (l.1), Rₐ₁ is H and R_{b1} is H. In some variations of formula (26), (27), (28), (29), (30), (31), or (32) described in the paragraphs above in which L is linker (l.1), Rₐ₁ is H and R_{b1} is CH₃. In some variations of formula (26), (27), (28), (29), (30), (31), or (32) described in the paragraphs above in which L is linker (l.1), Rₐ₁ and R_{b1} are

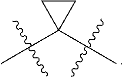 [SMILES: *C1(*)CC1 ; "."]

In some variations of formula (26), (27), (28), (29), (30), (31), or (32) described in the paragraphs above in which L is linker (l.1), Rₐ₁ and R_{b1} are

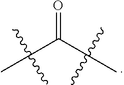 [SMILES: *C(*)=O ; "."]

In some variations of formula (26), (27), (28), (29), (30), (31), or (32) described in the paragraphs above in which L is linker (l.1), two of CRₐ₁R_{b1} are

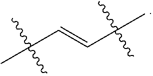 [SMILES: "." ; */C=C/*]

In some variations of formula (26), (27), (28), (29), (30), (31), or (32) described in the paragraphs above in which L is linker (l.1), Rₐ₂ is H and R_{b2} is H. In some variations of formula (26), (27), (28), (29), (30), (31), or (32) described in the paragraphs above in which L is linker (l.1), Rₐ₂ is H and R_{b2} is CH₃. In some variations of formula (26), (27), (28), (29), (30), (31), or (32) described in the paragraphs above in which L is linker (l.1), Rₐ₂ and R_{b2} are

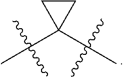 [SMILES: *C1(*)CC1 ; "."]

In some variations of formula (26), (27), (28), (29), (30), (31), or (32) described in the paragraphs above in which L is linker (l.1), Rₐ₂ and R_{b2} are

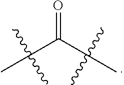 [SMILES: *C(*)=O ; "."]

In some variations of formula (26), (27), (28), (29), (30), (31), or (32) described in the paragraphs above in which L is linker (l.1), two of CRₐ₂R_{b2} are

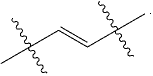 [SMILES: "." ; */C=C/*]

In some variations of formula (26), (27), (28), (29), (30), (31), or (32) described in the paragraphs above in which L is linker (l.1), R_{c1} and R_{d1} are both H. In some variations of formula (26), (27), (28), (29), (30), (31), or (32) described in the paragraphs above in which L is linker (l.1), Rₐ is H and R_{d1} is CH₃. In some variations of formula (26), (27), (28), (29), (30), (31), or (32) described in the paragraphs above in which L is linker (l.1), R_{c1} and R_{d1} are both CH₃. In some variations of formula (26), (27), (28), (29), (30), (31), or (32) described in the paragraphs above in which L is linker (l.1), R_{c1} and R_{d1} are

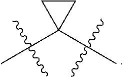 [SMILES: *C1(*)CC1 ; "."]

In some variations of formula (26), (27), (28), (29), (30), (31), or (32) described in the paragraphs above in which L is linker (l.1), R_{c1} and R_{d1} are

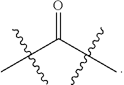 [SMILES: *C(*)=O ; "."]

In some variations of formula (26), (27), (28), (29), (30), (31), or (32) described in the paragraphs above in which L is linker (l.1), two of CR_{c1}R_{d1} are

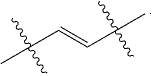 [SMILES: "." ; */C=C/*]

In some variations of formula (26), (27), (28), (29), (30), (31), or (32) described in the paragraphs above in which L is linker (l.1), R_{c2} and R_{d2} are both H. In some variations of formula (26), (27), (28), (29), (30), (31), or (32) described in the paragraphs above in which L is linker (l.1), R_{c2} is H and R_{d2} is CH₃. In some variations of formula (26), (27), (28), (29), (30), (31), or (32) described in the paragraphs above in which L is linker (l.1), R_{c2} and R_{d2} are both CH₃. In some variations of formula (26), (27), (28), (29), (30), (31), or (32) described in the paragraphs above in which L is linker (l.1), Rₑ₂ and R_{d2} are

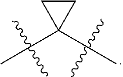 [SMILES: *C1(*)CC1 ; "."]

In some variations of formula (26), (27), (28), (29), (30), (31), or (32) described in the paragraphs above in which L is linker (l.1), Rₑ₂ and R_{d2} are

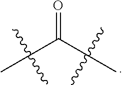 [SMILES: *C(*)=O ; "."]

In some variations of formula (26), (27), (28), (29), (30), (31), or (32) described in the paragraphs above in which L is linker (l.1), two of CR_{c2}R_{d2} are

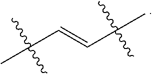 [SMILES: "." ; */C=C/*]

In some variations of formula (26), (27), (28), (29), (30), (31), or (32) described in the paragraphs above in which L is linker (l.1), ring A is

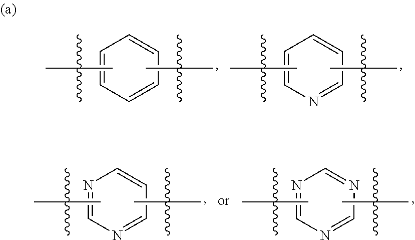

optionally substituted with halo or C1-C6 linear or branched alkyl;

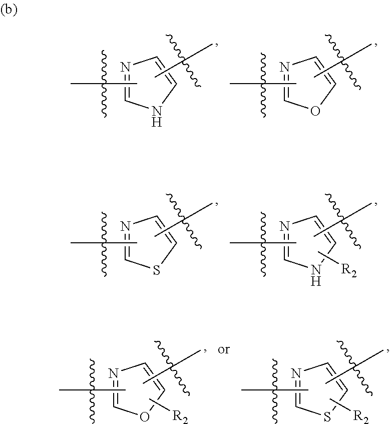

optionally substituted with halo or C1-C6 linear or branched alkyl; or

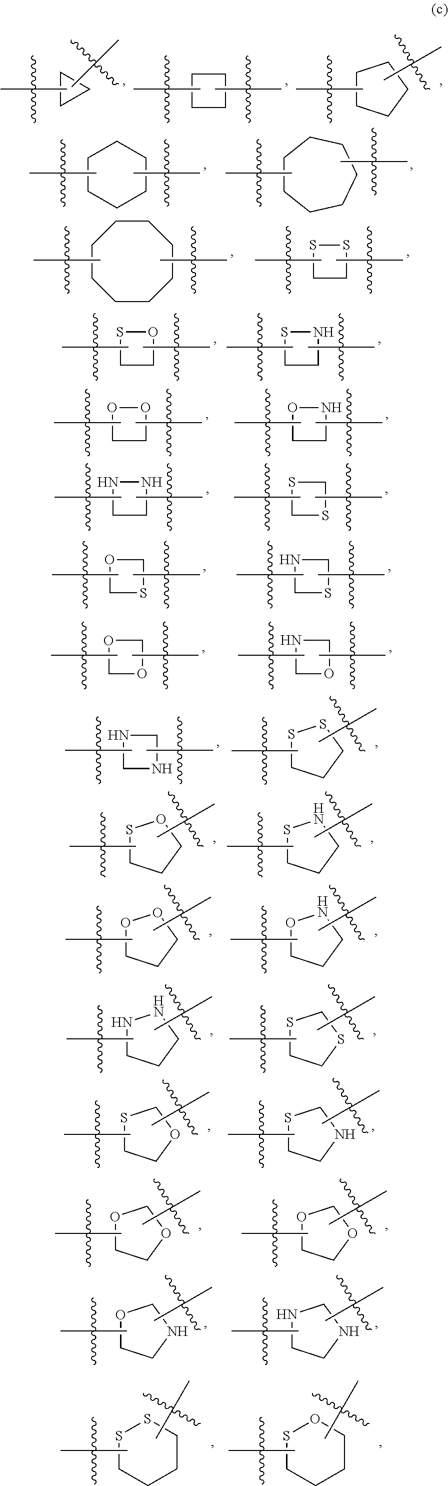

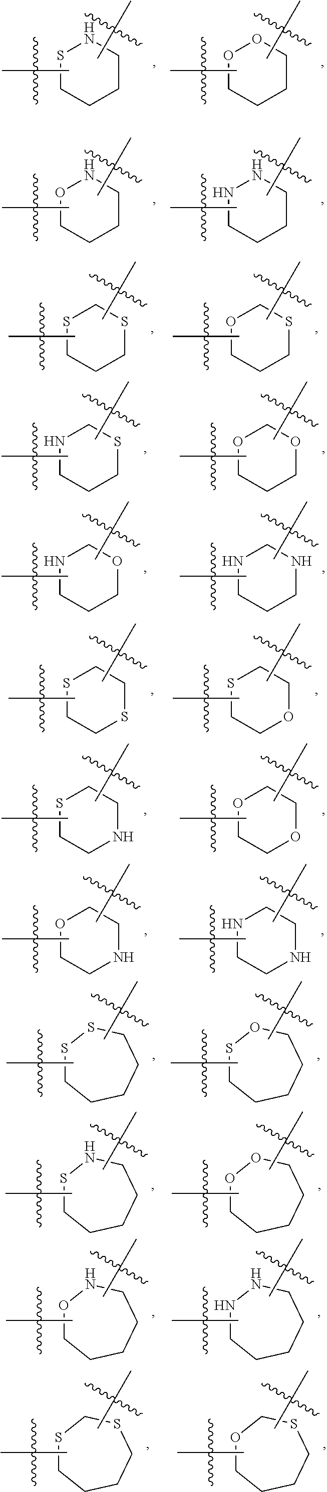

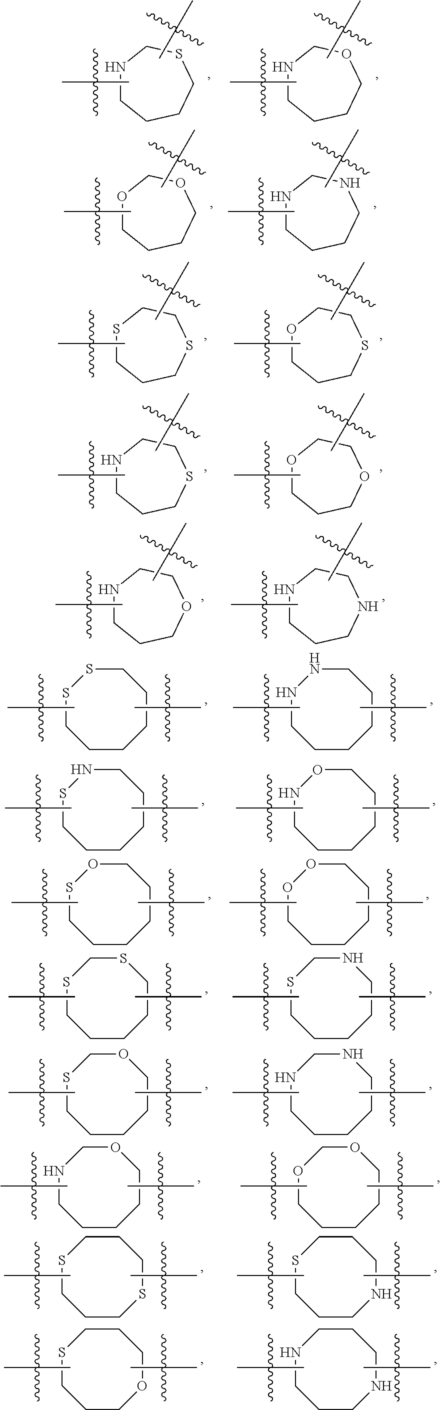

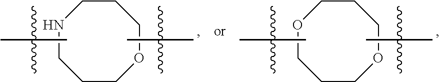

optionally substituted with halo or C1-C6 linear or branched alkyl.

In some variations of formula (26), (27), (28), (29), (30), (31), or (32) described in the paragraphs above in which L is linker (l.1), ring A is substituted with halo. In some variations, the halo is F, Br, I, or Cl.

In some variations of formula (26), (27), (28), (29), (30), (31), or (32) described in the paragraphs above in which L is linker (l.1), ring A is substituted with C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (26), (27), (28), (29), (30), (31), or (32) described in the paragraphs above in which L is linker (l.1), n is 1-4, 1-3, 1-3, 1-2, 2-4, 2-3, 3-4, 1, 2, 3, or 4.

In some variations of formula (26), (27), (28), (29), (30), (31), or (32), L is linker (l.2):

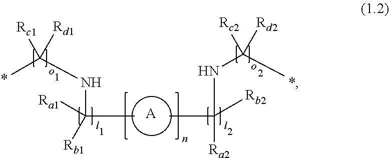

in which l₁, l₂, n, o₁ and o₂ independently are 1-4; ring A is aryl, heteroaryl, cycloalkyl, or heterocyclyl; for each independent instance of Rₐ₁ and R_{b1}, Rₐ₁ and R_{b1} (1) independently are H or CH₃, or (2) Rₐ₁ and R_{b1} independently are

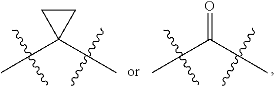

or (3) two of CRₐ₁R_{b1} are

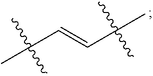 [SMILES: ";" ; */C=C/*]

for each independent instance of Rₐ₂ and R_{b2}, Rₐ₂ and R_{b2} (1) independently are H or CH₃, or (2) Rₐ₂ and R_{b2} independently are

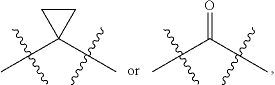

or (3) two of CRₐ₂R_{b2} are

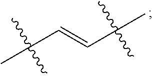 [SMILES: ";" ; */C=C/*]

for each independent instance of R_{c1} and R_{d1}, R_{c1} and R_{d1} (1) independently are H or CH₃, or (2) R_{c1} and R_{d1} independently are

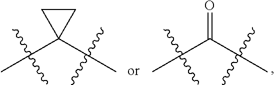

or (3) two of CR_{c1}R_{d1} are

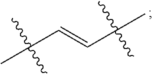 [SMILES: ";" ; */C=C/*]

for each independent instance of R_{c2} and R_{d2}, R_{c2} and R_{d2} (1) independently are H or CH₃, or (2) R_{c2} and R_{d2} independently are

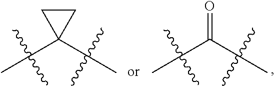

or (3) two of CR_{c2}R_{d2} are

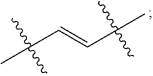 [SMILES: ";" ; */C=C/*]

and * is the attachment site for the cationic dye moiety D.

In some variations of formula (26), (27), (28), (29), (30), (31), or (32) in which L is linker (l.2), l₁ is 1-4, 1-3, 1-3, 1-2, 2-4, 2-3, 3-4, 1, 2, 3, or 4.

In some variations of formula (26), (27), (28), (29), (30), (31), or (32) described in the paragraphs above in which L is linker (l.2), l₂ is 1-4, 1-3, 1-3, 1-2, 2-4, 2-3, 3-4, 1, 2, 3, or 4.

In some variations of formula (26), (27), (28), (29), (30), (31), or (32) described in the paragraphs above in which L is linker (l.2), o₁ is 1-4, 1-3, 1-3, 1-2, 2-4, 2-3, 3-4, 1, 2, 3, or 4.

In some variations of formula (26), (27), (28), (29), (30), (31), or (32) described in the paragraphs above in which L is linker (l.2), o₂ is 1-4, 1-3, 1-3, 1-2, 2-4, 2-3, 3-4, 1, 2, 3, or 4.

In some variations of formula (26), (27), (28), (29), (30), (31), or (32) described in the paragraphs above in which L is linker (l.2), Rₐ₁ is H and R_{b1} is H. In some variations of formula (26), (27), (28), (29), (30), (31), or (32) described in the paragraphs above in which L is linker (l.2), Rₐ₁ is H and R_{b1} is CH₃. In some variations of formula (26), (27), (28), (29), (30), (31), or (32) described in the paragraphs above in which L is linker (l.2), Rₐ₁ and R_{b1} are

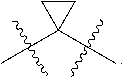 [SMILES: *C1(*)CC1 ; "."]

In some variations of formula (26), (27), (28), (29), (30), (31), or (32) described in the paragraphs above in which L is linker (l.2), Rₐ₁ and R_{b1} are

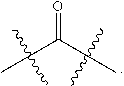 [SMILES: *C(*)=O ; "."]

In some variations of formula (26), (27), (28), (29), (30), (31), or (32) described in the paragraphs above in which L is linker (l.2), two of CRₐ₁R_{b1} are

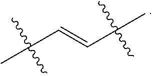 [SMILES: "." ; */C=C/*]

In some variations of formula (26), (27), (28), (29), (30), (31), or (32) described in the paragraphs above in which L is linker (l.2), Rₐ₂ is H and R_{b2} is H. In some variations of formula (26), (27), (28), (29), (30), (31), or (32) described in the paragraphs above in which L is linker (l.2), Rₐ₂ is H and R_{b2} is CH₃. In some variations of formula (26), (27), (28), (29), (30), (31), or (32) described in the paragraphs above in which L is linker (l.2), Rₐ₂ and R_{b2} are

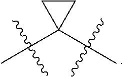 [SMILES: *C1(*)CC1 ; "."]

In some variations of formula (26), (27), (28), (29), (30), (31), or (32) described in the paragraphs above in which L is linker (l.2), Rₐ₂ and R_{b2} are

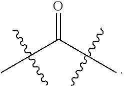 [SMILES: *C(*)=O ; "."]

In some variations of formula (26), (27), (28), (29), (30), (31), or (32) described in the paragraphs above in which L is linker (l.2), two of CRₐ₂R_{b2} are

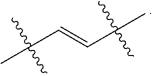 [SMILES: "." ; */C=C/*]

In some variations of formula (26), (27), (28), (29), (30), (31), or (32) described in the paragraphs above in which L is linker (l.2), R_{c1} and R_{d1} are both H. In some variations of formula (26), (27), (28), (29), (30), (31), or (32) described in the paragraphs above in which L is linker (l.2), R_{c1} is H and R_{d1} is CH₃. In some variations of formula (26), (27), (28), (29), (30), (31), or (32) described in the paragraphs above in which L is linker (l.2), R_{c1} and R_{d1} are both CH₃. In some variations of formula (26), (27), (28), (29), (30), (31), or (32) described in the paragraphs above in which L is linker (l.2), R_{c1} and R_{d1} are

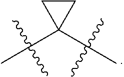 [SMILES: *C1(*)CC1 ; "."]

In some variations of formula (26), (27), (28), (29), (30), (31), or (32) described in the paragraphs above in which L is linker (l.2), R_{c1} and R_{d1} are

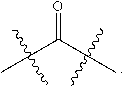 [SMILES: *C(*)=O ; "."]

In some variations of formula (26), (27), (28), (29), (30), (31), or (32) described in the paragraphs above in which L is linker (l.2), two of CR_{c1}R_{d1} are

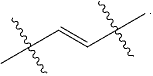 [SMILES: "." ; */C=C/*]

In some variations of formula (26), (27), (28), (29), (30), (31), or (32) described in the paragraphs above in which L is linker (l.2), R_{c1} and R_{d2} are both H. In some variations of formula (26), (27), (28), (29), (30), (31), or (32) described in the paragraphs above in which L is linker (l.2), Rₑ₂ is H and R_{d2} is CH₃. In some variations of formula (26), (27), (28), (29), (30), (31), or (32) described in the paragraphs above in which L is linker (l.2), R_{c2} and R_{d2} are both CH₃. In some variations of formula (26), (27), (28), (29), (30), (31), or (32) described in the paragraphs above in which L is linker (l.2), R_{c2} and R_{d2} are

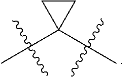 [SMILES: *C1(*)CC1 ; "."]

In some variations of formula (26), (27), (28), (29), (30), (31), or (32) described in the paragraphs above in which L is linker (l.2), R_{c2} and R_{d2} are

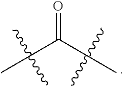 [SMILES: *C(*)=O ; "."]

In some variations of formula (26), (27), (28), (29), (30), (31), or (32) described in the paragraphs above in which L is linker (l.2), two of CR_{c2}R_{d2} are

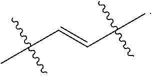 [SMILES: "." ; */C=C/*]

In some variations of formula (26), (27), (28), (29), (30), (31), or (32) described in the paragraphs above in which L is linker (l.2), ring A is

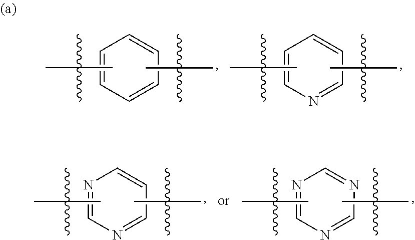

optionally substituted with halo or C1-C6 linear or branched alkyl;

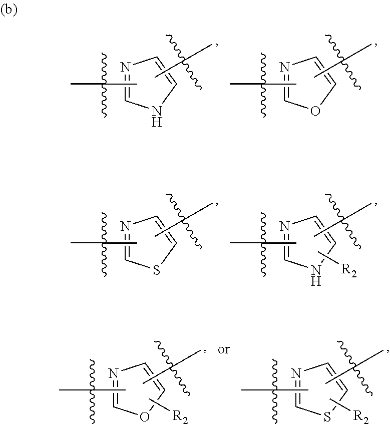

optionally substituted with halo or C1-C6 linear or branched alkyl; or

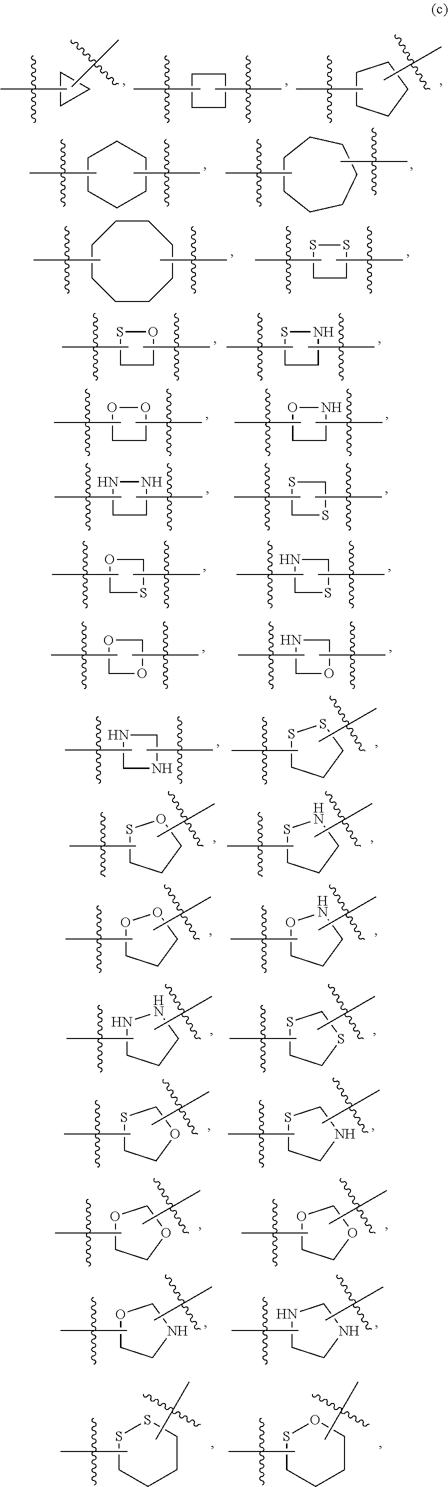

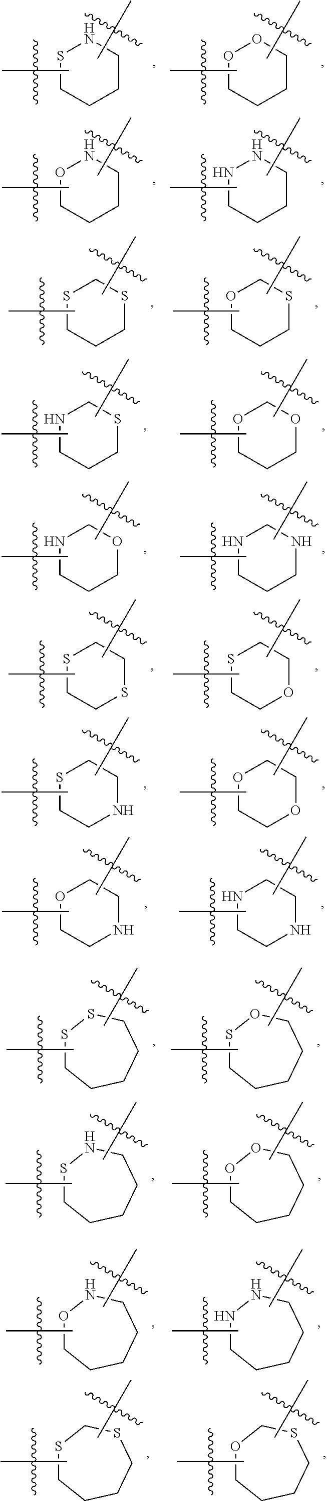

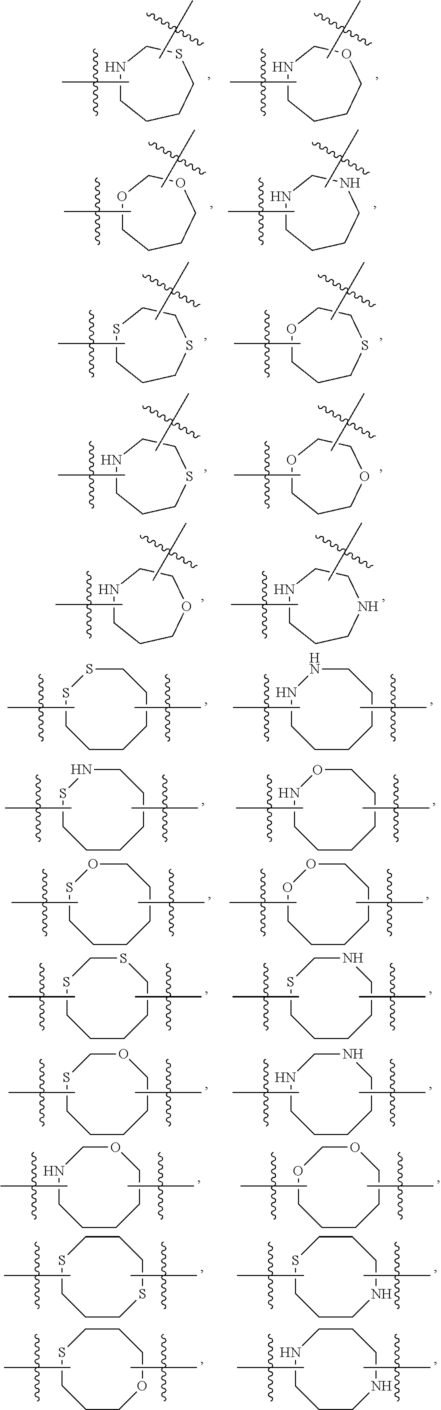

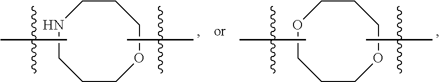

optionally substituted with halo or C1-C6 linear or branched alkyl.

In some variations of formula (26), (27), (28), (29), (30), (31), or (32) described in the paragraphs above in which L is linker (l.2), ring A is substituted with halo. In some variations, the halo is F, Br, I, or Cl.

In some variations of formula (26), (27), (28), (29), (30), (31), or (32) described in the paragraphs above in which L is linker (l.2), ring A is substituted with C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (26), (27), (28), (29), (30), (31), or (32) described in the paragraphs above in which L is linker (l.2), n is 1-4, 1-3, 1-3, 1-2, 2-4, 2-3, 3-4, 1, 2, 3, or 4.

In some variations of formula (26), (27), (28), (29), (30), (31), or (32), L is linker (m.1):

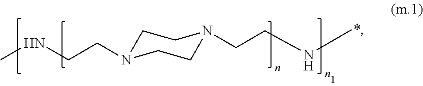

in which n is 0-6, n₁ is 1-4, and * is the attachment site for the cationic dye moiety D.

In some variations of formula (26), (27), (28), (29), (30), (31), or (32) in which L is linker (m.1), n is 0-6, 0-5, 0-4, 0-3, 0-2, 0-1, 1-6, 1-5, 1-4, 1-3, 1-2, 2-6, 2-5, 2-4, 2-3, 3-6, 3-5, 3-4, 4-6, 4-5, 5-6, 0, 1, 2, 3, 4, 5, or 6.

In some variations of formula (26), (27), (28), (29), (30), (31), or (32) described in the paragraphs above in which L is linker (m.1), n₁ is 1-4, 1-3, 1-2, 2-4, 2-3, 3-4, 1, 2, 3, or 4.

In some variations of formula (26), (27), (28), (29), (30), (31), or (32), L is linker (n.1):

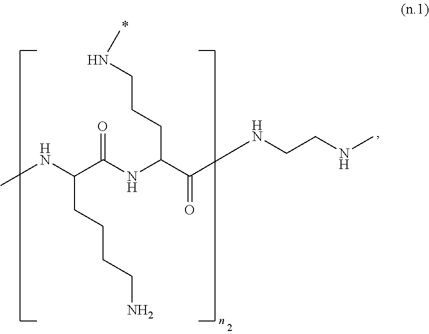

in which n₂ is 1-5 and * is the attachment site for the cationic dye moiety D.

In some variations of formula (26), (27), (28), (29), (30), (31), or (32) in which L is linker (n.1), n₂ is 1-5, 1-4, 1-3, 1-2, 2-5, 2-4, 2-3, 3-5, 3-4, 4-5, 1, 2, 3, 4, or 5.

In some variations of formula (26), (27), (28), (29), (30), (31), or (32), L is linker (n.2):

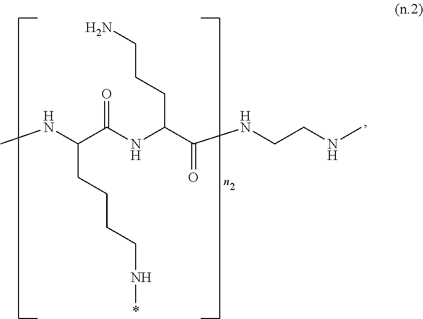

in which n₂ is 1-5 and * is the attachment site for the cationic dye moiety D.

In some variations of formula (26), (27), (28), (29), (30), (31), or (32) in which L is linker (n.2), n₂ is 1-5, 1-4, 1-3, 1-2, 2-5, 2-4, 2-3, 3-5, 3-4, 4-5, 1, 2, 3, 4, or 5.

In some variations of formula (26), (27), (28), (29), (30), (31), or (32), L is linker (o):

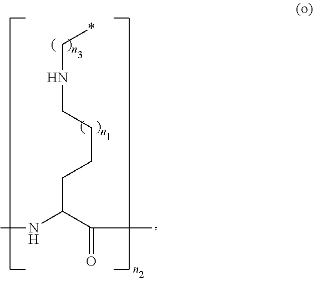

in which in which n₁ is 0-5, n₂ is 1-5, n3 is 0-5, and * is the attachment site for the cationic dye moiety D.

In some variations of formula (26), (27), (28), (29), (30), (31), or (32) in which L is linker (o), n₁ is 0-5, 0-4, 0-3, 0-2, 0-1, 1-5, 1-4, 1-3, 1-2, 2-5, 2-4, 2-3, 3-5, 3-4, 4-5, 0, 1, 2, 3, 4, or 5.

In some variations of formula (26), (27), (28), (29), (30), (31), or (32) described in the paragraphs above in which L is linker (o), n₂ is 1-5, 1-4, 1-3, 1-2, 2-5, 2-4, 2-3, 3-5, 3-4, 4-5, 1, 2, 3, 4, or 5.

In some variations of formula (26), (27), (28), (29), (30), (31), or (32) described in the paragraphs above in which L is linker (o), n₃ is 0-5, 0-4, 0-3, 0-2, 0-1, 1-5, 1-4, 1-3, 1-2, 2-5, 2-4, 2-3, 3-5, 3-4, 4-5, 0, 1, 2, 3, 4, or 5.

In some variations of formula (26), (27), (28), (29), (30), (31), or (32), L is linker (p):

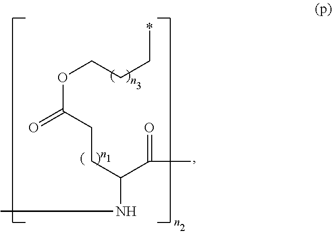

in which n₁ is 0-5, n₂ is 1-5, n3 is 0-5, and * is the attachment site for the cationic dye moiety D.

In some variations of formula (26), (27), (28), (29), (30), (31), or (32) described above in which L is linker (p), n₁ is 0-5, 0-4, 0-3, 0-2, 0-1, 1-5, 1-4, 1-3, 1-2, 2-5, 2-4, 2-3, 3-5, 3-4, 4-5, 0, 1, 2, 3, 4, or 5.

In some variations of formula (26), (27), (28), (29), (30), (31), or (32) described above in which L is linker (p), n₂ is 1-5, 1-4, 1-3, 1-2, 2-5, 2-4, 2-3, 3-5, 3-4, 4-5, 1, 2, 3, 4, or 5.

In some variations of formula (26), (27), (28), (29), (30), (31), or (32) described above in which L is linker (p), n₃ is 0-5, 0-4, 0-3, 0-2, 0-1, 1-5, 1-4, 1-3, 1-2, 2-5, 2-4, 2-3, 3-5, 3-4, 4-5, 0, 1, 2, 3, 4, or 5.

In some variations of formula (26), (27), (28), (29), (30), (31), or (32), L is linker (q):

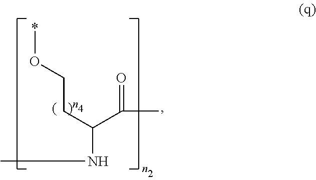

in which n₄ is 0-5, n₂ is 1-5, and * is the attachment site for the cationic dye moiety D.

In some variations of formula (26), (27), (28), (29), (30), (31), or (32) in which L is linker (o), n₄ is 0-5, 0-4, 0-3, 0-2, 0-1, 1-5, 1-4, 1-3, 1-2, 2-5, 2-4, 2-3, 3-5, 3-4, 4-5, 0, 1, 2, 3, 4, or 5.

In some variations of formula (26), (27), (28), (29), (30), (31), or (32) described above in which L is linker (q), n₂ is 1-5, 1-4, 1-3, 1-2, 2-5, 2-4, 2-3, 3-5, 3-4, 4-5, 1, 2, 3, 4, or 5.

In some variations of formula (26), (27), (28), (29), (30), (31), or (32), L is linker (r):

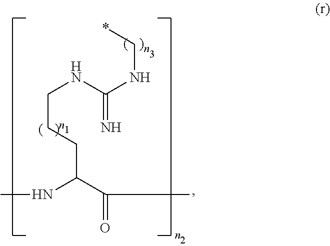

in which n₁ is 0-5, n₂ is 1-5, n3 is 0-5, and * is the attachment site for the cationic dye moiety D.

In some of variations of formula (26), (27), (28), (29), (30), (31), or (32) in which L is linker (r), n₁ is 0-5, 0-4, 0-3, 0-2, 0-1, 1-5, 1-4, 1-3, 1-2, 2-5, 2-4, 2-3, 3-5, 3-4, 4-5, 0, 1, 2, 3, 4, or 5.

In some of variations of formula (26), (27), (28), (29), (30), (31), or (32) described in the paragraphs above in which L is linker (r), n₂ is 1-5, 1-4, 1-3, 1-2, 2-5, 2-4, 2-3, 3-5, 3-4, 4-5, 1, 2, 3, 4, or 5.

In some of variations of formula (26), (27), (28), (29), (30), (31), or (32) in which L is linker (r), n3 is 0-5, 0-4, 0-3, 0-2, 0-1, 1-5, 1-4, 1-3, 1-2, 2-5, 2-4, 2-3, 3-5, 3-4, 4-5, 0, 1, 2, 3, 4, or 5.

In some variations of formula (26), (27), (28), (29), (30), (31), or (32), L is linker (s):

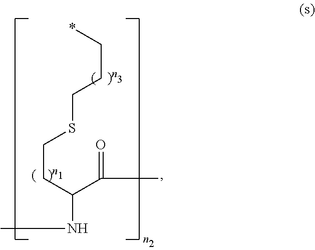

in which n₁ is 0-5, n₂ is 1-5, n₃ is 0-5, and * is the attachment site for the cationic dye moiety D.

In some variations of formula (26), (27), (28), (29), (30), (31), or (32) described above in which L is linker (s), n₁ is 0-5, 0-4, 0-3, 0-2, 0-1, 1-5, 1-4, 1-3, 1-2, 2-5, 2-4, 2-3, 3-5, 3-4, 4-5, 0, 1, 2, 3, 4, or 5.

In some variations of formula (26), (27), (28), (29), (30), (31), or (32) described above in which L is linker (s), n₂ is 1-5, 1-4, 1-3, 1-2, 2-5, 2-4, 2-3, 3-5, 3-4, 4-5, 1, 2, 3, 4, or 5.

In some variations of formula (26), (27), (28), (29), (30), (31), or (32) described above in which L is linker (s), n₃ is 0-5, 0-4, 0-3, 0-2, 0-1, 1-5, 1-4, 1-3, 1-2, 2-5, 2-4, 2-3, 3-5, 3-4, 4-5, 0, 1, 2, 3, 4, or 5.

In some variations of formula (26), (27), (28), (29), (30), (31), or (32) described above, L is absent.

In some variations of formula (26), (27), (28), (29), (30), (31), or (32) described in the paragraphs above, nx is 0-5, 0-4, 0-3, 0-2, 0-1, 1-5, 1-4, 1-3, 1-2, 2-5, 2-4, 2-3, 3-5, 3-4, 4-5, 0, 1, 2, 3, 4, or 5.

In some variations of formula (26), (27), (28), (29), (30), (31), or (32) described in the paragraphs above, ny is 1-5, 1-4, 1-3, 1-2, 2-5, 2-4, 2-3, 3-5, 3-4, 4-5, 1, 2, 3, 4, or 5.

In some variations of formula (26), (27), (28), (29), (30), (31), or (32) described in the paragraphs above, D is selected from the group consisting of safranin-O, toluidine blue, azure A, azure B, azure C, acridine orange, acriflavine, and methylene blue.

In some variations of formula (26), (27), (28), (29), (30), (31), or (32) described in the paragraphs above in which n₂ is greater than 1, each D is represented consecutively from left to right as D1, D2, D3, D4, and D5, as appropriate for the value of ny.

In some variations of formula (26), (27), (28), (29), (30), (31), or (32) described in the paragraphs above in which ny is 2, D1 and D2 are the same cationic dye moiety. In some variations of formula (26), (27), (28), (29), (30), (31), or (32) in which ny is 2, D1 and D2 are different cationic dye moieties. In some variations of formula (26), (27), (28), (29), (30), (31), or (32) in which ny is 2, D1 and D2 independently are selected from the group consisting of safranin-O, toluidine blue, azure A, azure B, azure C, acridine orange, acriflavine, and methylene blue.

In some variations of formula (26), (27), (28), (29), (30), (31), or (32) described in the paragraphs above in which ny is 3, D1, D2, and D3 are the same cationic dye moiety. In some variations of formula (26), (27), (28), (29), (30), (31), or (32) in which ny is 3, D1, D2, and D3 are different cationic dye moieties. In some variations of formula (26), (27), (28), (29), (30), (31), or (32) in which ny is 3, D1 and D2 and the same cationic dye moiety and D3 is a different cationic dye moiety. In some variations of formula (26), (27), (28), (29), (30), (31), or (32) in which ny is 3, D1 and D3 and the same cationic dye moiety and D2 is a different cationic dye moiety. In some variations of formula (26), (27), (28), (29), (30), (31), or (32) in which ny is 3, D2 and D3 and the same cationic dye moiety and D1 is a different cationic dye moiety. In some variations of formula (26), (27), (28), (29), (30), (31), or (32) in which ny is 3, D1, D2, and D3 independently are selected from the group consisting of safranin-O, toluidine blue, azure A, azure B, azure C, acridine orange, acriflavine, and methylene blue.

In some variations of formula (26), (27), (28), (29), (30), (31), or (32) described in the paragraphs above in which ny is 4, D1, D2, D3, and D4 are the same cationic dye moiety. In some variations of formula (26), (27), (28), (29), (30), (31), or (32) in which ny is 4, D1, D2, D3, and D4 are different cationic dye moieties. In some variations of formula (26), (27), (28), (29), (30), (31), or (32), D1 is a first cationic dye moiety and each of D2, D3, and D4 is the same second cationic dye moiety, wherein the first and second cationic dye moieties are different. In some variations of formula (26), (27), (28), (29), (30), (31), or (32), D2 is a first cationic dye moiety, and each of D1, D3, and D4 is a second cationic dye moiety, wherein the first and second cationic dye moieties are different. In some variations of formula (26), (27), (28), (29), (30), (31), or (32), D3 is a first cationic dye moiety, and each of D1, D2, and D4 is a second cationic dye moiety, wherein the first and second cationic dye moieties are different. In some variations of formula (26), (27), (28), (29), (30), (31), or (32), D4 is a first cationic dye moiety, and each of D1, D2, and D3 is a second cationic dye moiety, wherein the first and second cationic dye moieties are different. In some variations of formula (26), (27), (28), (29), (30), (31), or (32), each of D1 and D2 is the same first cationic dye moiety, and each of D3 and D4 is the same second cationic dye moiety, wherein the first and second cationic dye moieties are different. In some variations of formula (26), (27), (28), (29), (30), (31), or (32), each of D1 and D2 is the same first cationic dye moiety, D3 is a second cationic dye moiety, and D4 is a third cationic dye moiety, wherein the first, second, and third cationic dye moieties are different. In some variations of formula (26), (27), (28), (29), (30), (31), or (32), each of D1 and D3 is the same first cationic dye moiety, and each of D2 and D4 is the same second cationic dye moiety, wherein the first and second cationic dye moieties are different. In some variations of formula (26), (27), (28), (29), (30), (31), or (32), each of D1 and D3 is the same first cationic dye moiety, D2 is a second cationic dye moiety, and D4 is a third cationic dye moiety, wherein the first, second, and third cationic dye moieties are different. In some variations of formula (26), (27), (28), (29), (30), (31), or (32), each of D1 and D4 is the same first cationic dye moiety, and each of D2 and D3 is the same second cationic dye moiety, wherein the first and second cationic dye moieties are different. In some variations of formula (26), (27), (28), (29), (30), (31), or (32), each of D1 and D4 is the same first cationic dye moiety, D2 is a different second cationic dye moiety, and D3 is a different third cationic dye moiety, wherein the first, second, and third cationic dye moieties are different. In some variations of formula (26), (27), (28), (29), (30), (31), or (32), each of D2 and D3 is the same first cationic dye moiety, and each of D1 and D4 is the same second cationic dye moiety, wherein the first and second cationic dye moieties are different. In some variations of formula (26), (27), (28), (29), (30), (31), or (32), (26), (27), (28), (29), (30), (30), or (32) in which ny is 4, D1, D2, D3, and D4 independently are selected from the group consisting of safranin-O, toluidine blue, azure A, azure B, azure C, acridine orange, acriflavine, and methylene blue.

In some variations of formula (26), (27), (28), (29), (30), (31), or (32) described in the paragraphs above in which ny is 5, D1, D2, D3, D4, and D5 are different cationic dye moieties. In some variations of formula (26), (27), (28), (29), (30), (31), or (32) described in the paragraphs above in which ny is 5, D1, D2, D3, D4, and D5 are the same cationic dye moiety. In some variations of formula (26), (27), (28), (29), (30), (31), or (32) described in the paragraphs above in which ny is 5, D1 and D2 are a first cationic dye moiety, D3 is a second cationic dye moiety, D4 is a third cationic dye moiety, and D5 is a fourth cationic dye moiety, wherein the first, second, third, and fourth cationic dye moieties are different. In some variations of formula (26), (27), (28), (29), (30), (31), or (32) described in the paragraphs above in which ny is 5, D1 and D2 are a first cationic dye moiety, D3 is a second cationic dye moiety, and D4 and D5 each are a third cationic dye moiety, wherein the first, second, and third cationic dye moieties are different. In some variations of formula (26), (27), (28), (29), (30), (31), or (32) described in the paragraphs above in which ny is 5, D1 and D2 are a first cationic dye moiety, D3 and D4 are a second cationic dye moiety, and D5 is a third cationic dye moiety, wherein the first, second, and third cationic dye moieties are different. In some variations of formula (26), (27), (28), (29), (30), (31), or (32) described in the paragraphs above in which ny is 5, D1 and D2 are a first cationic dye moiety, D3 and D5 are a second cationic dye moiety, and D4 is a third cationic dye moiety, wherein the first, second, and third cationic dye moieties are different.

In some variations of formula (26), (27), (28), (29), (30), (31), or (32) described in the paragraphs above in which ny is 5, D1 and D3 are a first cationic dye moiety, D2 is a second cationic dye moiety, D4 is a third cationic dye moiety, and D5 is a fourth cationic dye moiety, wherein the first, second, third, and fourth cationic dye moieties are different. In some variations of formula (26), (27), (28), (29), (30), (31), or (32) described in the paragraphs above in which ny is 5, D1 and D3 are a first cationic dye moiety, D5 is a second cationic dye moiety, and D2 and D4 each are a third cationic dye moiety, wherein the first, second, and third cationic dye moieties are different. In some variations of formula (26), (27), (28), (29), (30), (31), or (32) described in the paragraphs above in which ny is 5, D1 and D3 are a first cationic dye moiety, D2 and D5 are a second cationic dye moiety, and D4 is a third cationic dye moiety, wherein the first, second, and third cationic dye moieties are different. In some variations of formula (26), (27), (28), (29), (30), (31), or (32) described in the paragraphs above in which ny is 5, D1 and D3 are a first cationic dye moiety, D4 and D5 are a second cationic dye moiety, and D3 is a third cationic dye moiety, wherein the first, second, and third cationic dye moieties are different.

In some variations of formula (26), (27), (28), (29), (30), (31), or (32) described in the paragraphs above in which ny is 5, D1 and D4 are a first cationic dye moiety, D2 is a second cationic dye moiety, D3 is a third cationic dye moiety, and D5 is a fourth cationic dye moiety, wherein the first, second, third, and fourth cationic dye moieties are different. In some variations of formula (26), (27), (28), (29), (30), (31), or (32) described in the paragraphs above in which ny is 5, D1 and D4 are a first cationic dye moiety, D3 is a second cationic dye moiety, and D2 and D5 each are a third cationic dye moiety, wherein the first, second, and third cationic dye moieties are different. In some variations of formula (26), (27), (28), (29), (30), (31), or (32) described in the paragraphs above in which ny is 5, D1 and D4 are a first cationic dye moiety, D2 and D3 are a second cationic dye moiety, and D5 is a third cationic dye moiety, wherein the first, second, and third cationic dye moieties are different. In some variations of formula (26), (27), (28), (29), (30), (31), or (32) described in the paragraphs above in which ny is 5, D1 and D4 are a first cationic dye moiety, D3 and D5 are a second cationic dye moiety, and D2 is a third cationic dye moiety, wherein the first, second, and third cationic dye moieties are different.

In some variations of formula (26), (27), (28), (29), (30), (31), or (32) described in the paragraphs above in which ny is 5, D1 and D5 are a first cationic dye moiety, D2 is a second cationic dye moiety, D3 is a third cationic dye moiety, and D4 is a fourth cationic dye moiety, wherein the first, second, third, and fourth cationic dye moieties are different. In some variations of formula (26), (27), (28), (29), (30), (31), or (32) described in the paragraphs above in which ny is 5, D1 and D5 are a first cationic dye moiety, D4 is a second cationic dye moiety, and D2 and D3 each are a third cationic dye moiety, wherein the first, second, and third cationic dye moieties are different. In some variations of formula (26), (27), (28), (29), (30), (31), or (32) described in the paragraphs above in which ny is 5, D1 and D5 are a first cationic dye moiety, D3 and D4 are a second cationic dye moiety, and D2 is a third cationic dye moiety, wherein the first, second, and third cationic dye moieties are different. In some variations of formula (26), (27), (28), (29), (30), (31), or (32) described in the paragraphs above in which ny is 5, D1 and D5 are a first cationic dye moiety, D2 and D4 are a second cationic dye moiety, and D3 is a third cationic dye moiety, wherein the first, second, and third cationic dye moieties are different.

In some variations of formula (26), (27), (28), (29), (30), (31), or (32) described in the paragraphs above in which ny is 5, D2 and D3 are a first cationic dye moiety, D1 is a second cationic dye moiety, D4 is a third cationic dye moiety, and D5 is a fourth cationic dye moiety, wherein the first, second, third, and fourth cationic dye moieties are different. In some variations of formula (26), (27), (28), (29), (30), (31), or (32) described in the paragraphs above in which ny is 5, D2 and D3 are a first cationic dye moiety, D1 is a second cationic dye moiety, and D4 and D3 each are a third cationic dye moiety, wherein the first, second, and third cationic dye moieties are different.

In some variations of formula (26), (27), (28), (29), (30), (31), or (32) described in the paragraphs above in which ny is 5, D2 and D4 are a first cationic dye moiety, D1 is a second cationic dye moiety, D3 is a third cationic dye moiety, and D5 is a fourth cationic dye moiety, wherein the first, second, third, and fourth cationic dye moieties are different. In some variations of formula (26), (27), (28), (29), (30), (31), or (32) described in the paragraphs above in which ny is 5, D2 and D4 are a first cationic dye moiety, D1 is a second cationic dye moiety, and D3 and D5 each are a third cationic dye moiety, wherein the first, second, and third cationic dye moieties are different.

In some variations of formula (26), (27), (28), (29), (30), (31), or (32) described in the paragraphs above in which ny is 5, D2 and D5 are a first cationic dye moiety, D1 is a second cationic dye moiety, D3 is a third cationic dye moiety, and D4 is a fourth cationic dye moiety, wherein the first, second, third, and fourth cationic dye moieties are different. In some variations of formula (26), (27), (28), (29), (30), (31), or (32) described in the paragraphs above in which ny is 5, D2 and D5 are a first cationic dye moiety, D1 is a second cationic dye moiety, and D3 and D4 each are a third cationic dye moiety, wherein the first, second, and third cationic dye moieties are different.

In some variations of formula (26), (27), (28), (29), (30), (31), or (32) described in the paragraphs above in which ny is 5, D3 and D4 are a first cationic dye moiety, D1 is a second cationic dye moiety, D2 is a third cationic dye moiety, and D5 is a fourth cationic dye moiety, wherein the first, second, third, and fourth cationic dye moieties are different.

In some variations of formula (26), (27), (28), (29), (30), (31), or (32) described in the paragraphs above in which ny is 5, D4 and D5 are a first cationic dye moiety, D1 is a second cationic dye moiety, D2 is a third cationic dye moiety, and D3 is a fourth cationic dye moiety, wherein the first, second, third, and fourth cationic dye moieties are different.

In some variations of formula (26), (27), (28), (29), (30), (31), or (32) described in the paragraphs above in which ny is 5, D1, D2, and D3 are a first cationic dye moiety, D4 is a second cationic dye moiety, and D5 is a third cationic dye moiety, wherein the first, second, and third cationic dye moieties are different. In some variations of formula (26), (27), (28), (29), (30), (31), or (32) described in the paragraphs above in which ny is 5, D1, D2, and D3 are a first cationic dye moiety, and D4 and D5 are a second cationic dye moiety, wherein the first and second cationic dye moieties are different.

In some variations of formula (26), (27), (28), (29), (30), (31), or (32) described in the paragraphs above in which ny is 5, D2, D3, and D4 are a first cationic dye moiety, D1 is a second cationic dye moiety, and D5 is a third cationic dye moiety, wherein the first, second, and third cationic dye moieties are different. In some variations of formula (26), (27), (28), (29), (30), (31), or (32) described in the paragraphs above in which ny is 5, D2, D3, and D4 are a first cationic dye moiety, and D1 and D5 are a second cationic dye moiety, wherein the first and second cationic dye moieties are different.

In some variations of formula (26), (27), (28), (29), (30), (31), or (32) described in the paragraphs above in which ny is 5, D3, D4, and D5 are a first cationic dye moiety, D1 is a second cationic dye moiety, and D2 is a third cationic dye moiety, wherein the first, second, and third cationic dye moieties are different. In some variations of formula (26), (27), (28), (29), (30), (31), or (32) described in the paragraphs above in which ny is 5, D3, D4, and D5 are a first cationic dye moiety, and D1 and D2 are a second cationic dye moiety, wherein the first and second cationic dye moieties are different.

In some variations of formula (26), (27), (28), (29), (30), (31), or (32) described in the paragraphs above in which ny is 5, D1, D4, and D5 are a first cationic dye moiety, D2 is a second cationic dye moiety, and D3 is a third cationic dye moiety, wherein the first, second, and third cationic dye moieties are different. In some variations of formula (26), (27), (28), (29), (30), (31), or (32) described in the paragraphs above in which ny is 5, D1, D4, and D5 are a first cationic dye moiety, and D2 and D3 are a second cationic dye moiety, wherein the first and second cationic dye moieties are different.

In some variations of formula (26), (27), (28), (29), (30), (31), or (32) described in the paragraphs above in which ny is 5, D1, D2, and D5 are a first cationic dye moiety, D3 is a second cationic dye moiety, and D4 is a third cationic dye moiety, wherein the first, second, and third cationic dye moieties are different. In some variations of formula (26), (27), (28), (29), (30), (31), or (32) described in the paragraphs above in which ny is 5, D1, D2, and D5 are a first cationic dye moiety, and D3 and D4 are a second cationic dye moiety, wherein the first and second cationic dye moieties are different.

In some variations of formula (26), (27), (28), (29), (30), (31), or (32) described in the paragraphs above in which ny is 5, D1 is a first cationic dye moiety, and each of D2, D3, D4, and D5 is a second cationic dye moiety, wherein the first and second cationic dye moieties are different. In some variations of formula (26), (27), (28), (29), (30), (31), or (32) described in the paragraphs above in which ny is 5, D2 is a first cationic dye moiety, and each of D1, D3, D4, and D5 is a second cationic dye moiety, wherein the first and second cationic dye moieties are different. In some variations of formula (26), (27), (28), (29), (30), (31), or (32) described in the paragraphs above in which ny is 5, D3 is a first cationic dye moiety, and each of D1, D2, D4, and D5 is a second cationic dye moiety, wherein the first and second cationic dye moieties are different. In some variations of formula (26), (27), (28), (29), (30), (31), or (32) described in the paragraphs above in which ny is 5, D4 is a first cationic dye moiety, and each of D1, D2, D3, and D5 is a second cationic dye moiety, wherein the first and second cationic dye moieties are different. In some variations of formula (26), (27), (28), (29), (30), (31), or (32) described in the paragraphs above in which ny is 5, D5 is a first cationic dye moiety, and each of D1, D2, D3, and D4 is a second cationic dye moiety, wherein the first and second cationic dye moieties are different.

In some variations of formula (26), (27), (28), (29), (30), (31), or (32) described in the paragraphs above in which ny is 5, D1, D2, D3, D4, and D5 independently are selected from the group consisting of safranin-O, toluidine blue, azure A, azure B, azure C, acridine orange, acriflavine, and methylene blue.

In some variations of formula (26), (27), (28), (29), (30), (31), or (32) described in the paragraphs above, D1 is safranin-O. In some variations of formula (26), (27), (28), (29), (30), (31), or (32) described in the paragraphs above, D2 is safranin-O. In some variations of formula (26), (27), (28), (29), (30), (31), or (32) described in the paragraphs above, D3 is safranin-O. In some variations of formula (26), (27), (28), (29), (30), (31), or (32) described in the paragraphs above, D4 is safranin-O. In some variations of formula (26), (27), (28), (29), (30), (31), or (32) described in the paragraphs above, D1 and D2 are safranin-O. In some variations of formula (26), (27), (28), (29), (30), (31), or (32) described in the paragraphs above, D1 and D3 are safranin-O. In some variations of formula (26), (27), (28), (29), (30), (31), or (32) described in the paragraphs above, D1 and D4 are safranin-O. In some variations of formula (26), (27), (28), (29), (30), (31), or (32) described in the paragraphs above, D2 and D3 are safranin-O. In some variations of formula (26), (27), (28), (29), (30), (31), or (32) described in the paragraphs above, D2 and D4 are safranin-O. In some variations of formula (26), (27), (28), (29), (30), (31), or (32) described in the paragraphs above, D3 and D4 are safranin-O. In some variations of formula (26), (27), (28), (29), (30), (31), or (32) described in the paragraphs above, D1, D2, and D3 are safranin-O. In some variations of formula (26), (27), (28), (29), (30), (31), or (32) described in the paragraphs above, D1, D2, and D4 are safranin-O. In some variations of formula (26), (27), (28), (29), (30), (31), or (32) described in the paragraphs above, D1, D3, and D4 are safranin-O. In some variations of formula (26), (27), (28), (29), (30), (31), or (32) described in the paragraphs above, D2, D3, and D4 are safranin-O. In some variations of formula (26), (27), (28), (29), (30), (31), or (32) described in the paragraphs above, D1, D2, D3, and D4 are safranin-0.

In some variations of formula (26), (27), (28), (29), (30), (31), or (32) described in the paragraph above, the pendant phenyl ring of D1 is unsubstituted. In some variations of formula (26), (27), (28), (29), (30), (31), or (32) described in the paragraph above, the pendant phenyl ring of D1 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D1 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D1 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (26), (27), (28), (29), (30), (31), or (32) described in the two paragraphs above, the pendant phenyl ring of D2 is unsubstituted. In some variations of formula (26), (27), (28), (29), (30), (31), or (32) described in the paragraph above, the pendant phenyl ring of D2 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D2 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D2 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (26), (27), (28), (29), (30), (31), or (32) described in the three paragraphs above, the pendant phenyl ring of D3 is unsubstituted. In some variations of formula (26), (27), (28), (29), (30), (31), or (32) described in the paragraph above, the pendant phenyl ring of D3 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D3 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D3 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (26), (27), (28), (29), (30), (31), or (32) described in the three paragraphs above, the pendant phenyl ring of D4 is unsubstituted. In some variations of formula (26), (27), (28), (29), (30), (31), or (32) described in the paragraph above, the pendant phenyl ring of D4 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D4 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D4 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations, cationic dye multimers are linear, as illustrated by formula (33):

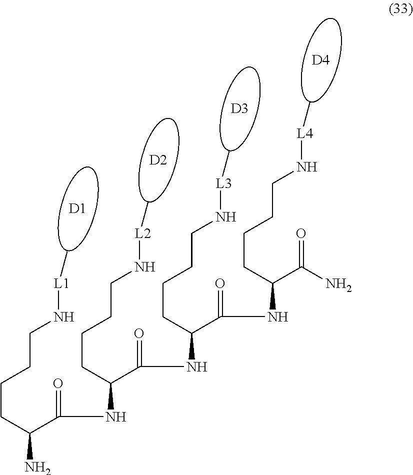

wherein each of D1, D2, D3, and D4 is a cationic dye moiety and one or more of L1, L2, L3, and L4 are absent or each of L1, L2, L3, and L4 is a linker independently selected from linker (a.1), linker (a.2), linker (b.1), linker (c.1), linker (c.2), linker (d), linker (e.1), linker (f.1), linker (f.2), linker (g.1), linker (g.2), linker (h.1), linker (h.2), linker (i.1), linker (i.2), linker (j.1), linker (j.2), linker (k), linker (l.1), linker (l.2), is linker (m.1), linker (n.1), linker (n.2), linker (o), linker (p), linker (q), linker (r), and linker (s), described above.

In some variations of formula (33), each of D1, D2, D3, and D4 is a different cationic dye moiety. In some variations of formula (33), each of D1, D2, D3, and D4 is the same cationic dye moiety. In some variations of formula (33), D1 is a first cationic dye moiety and each of D2, D3, and D4 is the same second cationic dye moiety, wherein the first and second cationic dye moieties are different. In some variations of formula (33), D2 is a first cationic dye moiety, and each of D1, D3, and D4 is a second cationic dye moiety, wherein the first and second cationic dye moieties are different. In some variations of formula (33), D3 is a first cationic dye moiety, and each of D1, D2, and D4 is a second cationic dye moiety, wherein the first and second cationic dye moieties are different. In some variations of formula (33), D4 is a first cationic dye moiety, and each of D1, D2, and D3 is a second cationic dye moiety, wherein the first and second cationic dye moieties are different. In some variations of formula (33), each of D1 and D2 is the same first cationic dye moiety, and each of D3 and D4 is the same second cationic dye moiety, wherein the first and second cationic dye moieties are different. In some variations of formula (33), each of D1 and D2 is the same first cationic dye moiety, D3 is a second cationic dye moiety, and D4 is a third cationic dye moiety, wherein the first, second, and third cationic dye moieties are different. In some variations of formula (33), each of D1 and D3 is the same first cationic dye moiety, and each of D2 and D4 is the same second cationic dye moiety, wherein the first and second cationic dye moieties are different. In some variations of formula (33), each of D1 and D3 is the same first cationic dye moiety, D2 is a second cationic dye moiety, and D4 is a third cationic dye moiety, wherein the first, second, and third cationic dye moieties are different. In some variations of formula (33), each of D1 and D4 is the same first cationic dye moiety, and each of D2 and D3 is the same second cationic dye moiety, wherein the first and second cationic dye moieties are different. In some variations of formula (33), each of D1 and D4 is the same first cationic dye moiety, D2 is a different second cationic dye moiety, and D3 is a different third cationic dye moiety, wherein the first, second, and third cationic dye moieties are different. In some variations of formula (33), each of D2 and D3 is the same first cationic dye moiety, and each of D1 and D4 is the same second cationic dye moiety, wherein the first and second cationic dye moieties are different.

In some variations of formula (33) described in the paragraphs above, each of D1, D2, D3, and D4 is independently are selected from the group consisting of safranin-O, toluidine blue, azure A, azure B, azure C, acridine orange, acriflavine, and methylene blue.

In some variations of formula (33) described in the paragraphs above, at least one of L1, L2, L3, and L4 is linker (a.1):

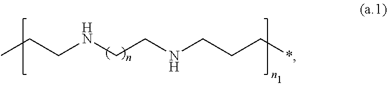

in which n is 1-6, n₁ is 1-4, and * is the attachment site for the cationic dye moiety D.

In some variations of formula (33) in which at least one of L1, L2, L3, and L4 is linker (a.1), n is 1-6, 1-5, 1-4, 1-3, 1-2, 2-6, 2-5, 2-4, 2-3, 3-6, 3-5, 3-4, 4-6, 4-5, 5-6, 1, 2, 3, 4, 5, or 6.

In some variations of formula (33) described in the paragraphs above in which at least one of L1, L2, L3, and L4 is linker (a.1), n₁ is 1-4, 1-3, 1-2, 2-4, 2-3, 3-4, 1, 2, 3, or 4.

In some variations of formula (33) described in the paragraphs above, L1 is linker (a.1). In some variations of formula (33) described in the paragraphs above, L2 is linker (a.1). In some variations of formula (33) described in the paragraphs above, L3 is linker (a.1). In some variations of formula (33) described in the paragraphs above, L4 is linker (a.1). In some variations of formula (33) described in the paragraphs above, L1 and L2 are linker (a.1). In some variations of formula (33) described in the paragraphs above, L1 and L3 are linker (a.1). In some variations of formula (33) described in the paragraphs above, L1 and L4 are linker (a.1). In some variations of formula (33) described in the paragraphs above, L2 and L3 are linker (a.1). In some variations of formula (33) described in the paragraphs above, L2 and L4 are linker (a.1). In some variations of formula (33) described in the paragraphs above, L3 and L4 are linker (a.1). In some variations of formula (33) described in the paragraphs above, L1, L2, and L3 are linker (a.1). In some variations of formula (33) described in the paragraphs above, L1, L2, and L4 are linker (a.1). In some variations of formula (33) described in the paragraphs above, L1, L3, and L4 are linker (a.1). In some variations of formula (33) described in the paragraphs above, L2, L3, and L4 are linker (a.1). In some variations of formula (33) described in the paragraphs above, L1, L2, L3, and L4 are linker (a.1).

In some variations of formula (33) described in the paragraphs above, at least one of L1, L2, L3, and L4 is linker (a.2):

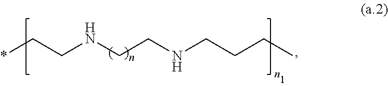

in which n is 1-6, n₁ is 1-4, and * is the attachment site for the cationic dye moiety D.

In some variations of formula (33) in which at least one of L1, L2, L3, and L4 is linker (a.2), n is 1-6, 1-5, 1-4, 1-3, 1-2, 2-6, 2-5, 2-4, 2-3, 3-6, 3-5, 3-4, 4-6, 4-5, 5-6, 1, 2, 3, 4, 5, or 6.

In some variations of formula (33) described in the paragraphs above in which at least one of L1, L2, L3, and L4 is linker (a.2), n₁ is 1-4, 1-3, 1-2, 2-4, 2-3, 3-4, 1, 2, 3, or 4.

In some variations of formula (33) described in the paragraphs above, L1 is linker (a.2). In some variations of formula (33) described in the paragraphs above, L2 is linker (a.2). In some variations of formula (33) described in the paragraphs above, L3 is linker (a.2). In some variations of formula (33) described in the paragraphs above, L4 is linker (a.2). In some variations of formula (33) described in the paragraphs above, L1 and L2 are linker (a.2). In some variations of formula (33) described in the paragraphs above, L1 and L3 are linker (a.2). In some variations of formula (33) described in the paragraphs above, L1 and L4 are linker (a.2). In some variations of formula (33) described in the paragraphs above, L2 and L3 are linker (a.2). In some variations of formula (33) described in the paragraphs above, L2 and L4 are linker (a.2). In some variations of formula (33) described in the paragraphs above, L3 and L4 are linker (a.2). In some variations of formula (33) described in the paragraphs above, L1, L2, and L3 are linker (a.2). In some variations of formula (33) described in the paragraphs above, L1, L2, and L4 are linker (a.2). In some variations of formula (33) described in the paragraphs above, L1, L3, and L4 are linker (a.2). In some variations of formula (33) described in the paragraphs above, L2, L3, and L4 are linker (a.2). In some variations of formula (33) described in the paragraphs above, L1, L2, L3, and L4 are linker (a.2).

In some variations of formula (33) described in the paragraphs above, at least one of L1, L2, L3, and L4 is linker (b.1):

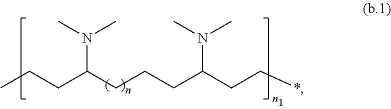

in which n is 0-6, n₁ is 1-4, and * is the attachment site for the cationic dye moiety D.

In some variations of formula (33) in which at least one of L1, L2, L3, and L4 is linker (b.1), n is 0-6, 0-5, 0-4, 0-3, 0-2, 0-1, 1-6, 1-5, 1-4, 1-3, 1-2, 2-6, 2-5, 2-4, 2-3, 3-6, 3-5, 3-4, 4-6, 4-5, 5-6, 0, 1, 2, 3, 4, 5, or 6.

In some variations of formula (33) described in the paragraphs above in which at least one of L1, L2, L3, and L4 is linker (b.1), n₁ is 1-4, 1-3, 1-2, 2-4, 2-3, 3-4, 1, 2, 3, or 4.

In some variations of formula (33) described in the paragraphs above, L1 is linker (b.1). In some variations of formula (33) described in the paragraphs above, L2 is linker (b.1). In some variations of formula (33) described in the paragraphs above, L3 is linker (b.1). In some variations of formula (33) described in the paragraphs above, L4 is linker (b.1). In some variations of formula (33) described in the paragraphs above, L1 and L2 are linker (b.1). In some variations of formula (33) described in the paragraphs above, L1 and L3 are linker (b.1). In some variations of formula (33) described in the paragraphs above, L1 and L4 are linker (b.1). In some variations of formula (33) described in the paragraphs above, L2 and L3 are linker (b.1). In some variations of formula (33) described in the paragraphs above, L2 and L4 are linker (b.1). In some variations of formula (33) described in the paragraphs above, L3 and L4 are linker (b.1). In some variations of formula (33) described in the paragraphs above, L1, L2, and L3 are linker (b.1). In some variations of formula (33) described in the paragraphs above, L1, L2, and L4 are linker (b.1). In some variations of formula (33) described in the paragraphs above, L1, L3, and L4 are linker (b.1). In some variations of formula (33) described in the paragraphs above, L2, L3, and L4 are linker (b.1). In some variations of formula (33) described in the paragraphs above, L1, L2, L3, and L4 are linker (b.1).

In some variations of formula (33) described in the paragraphs above, at least one of L1, L2, L3, and L4 is linker (c.1):

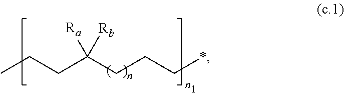

in which n is 0-6, n₁ is 1-4, either (1) Rₐ and R_{b} independently are H or CH₃ or (2) Rₐ and R_{b} are

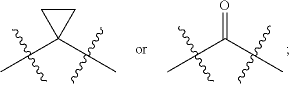

or (3) two of CRₐR_{b} are

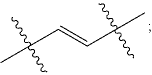 [SMILES: */C=C/*]

and * is the attachment site for the cationic dye moiety D.

In some variations of formula (33) in which at least one of L1, L2, L3, and L4 is linker (c.1), n is 0-6, 0-5, 0-4, 0-3, 0-2, 0-1, 1-6, 1-5, 1-4, 1-3, 1-2, 2-6, 2-5, 2-4, 2-3, 3-6, 3-5, 3-4, 4-6, 4-5, 5-6, 0, 1, 2, 3, 4, 5, or 6.

In some variations of formula (33) described in the paragraphs above in which at least one of L1, L2, L3, and L4 is linker (c.1), n₁ is 1-4, 1-3, 1-2, 2-4, 2-3, 3-4, 1, 2, 3, or 4.

In some variations of formula (33) described in the paragraphs above in which at least one of L1, L2, L3, and L4 is linker (c.1), Rₐ is H and R_{b} is H. In some variations of formula (33) described in the paragraphs above in which at least one of L1, L2, L3, and L4 is linker (c.1), Rₐ is H and R_{b} is CH₃. In some variations of formula (33) described in the paragraphs above in which at least one of L1, L2, L3, and L4 is linker (c.1), Rₐ and R_{b} are

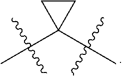 [SMILES: *C1(*)CC1]

In some variations of formula (33) described in the paragraphs above in which at least one of L1, L2, L3, and L4 is linker (c.1), Rₐ and R_{b} are

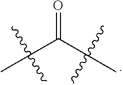 [SMILES: *C(*)=O]

In some variations of formula (33) described in the paragraphs above in which at least one of L1, L2, L3, and L4 is linker (c.1), two of CRₐR_{b} are

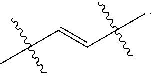 [SMILES: "." ; */C=C/*]

In some variations of formula (33) described in the paragraphs above in which at least one of L1, L2, L3, and L4 is linker (c.1), L is

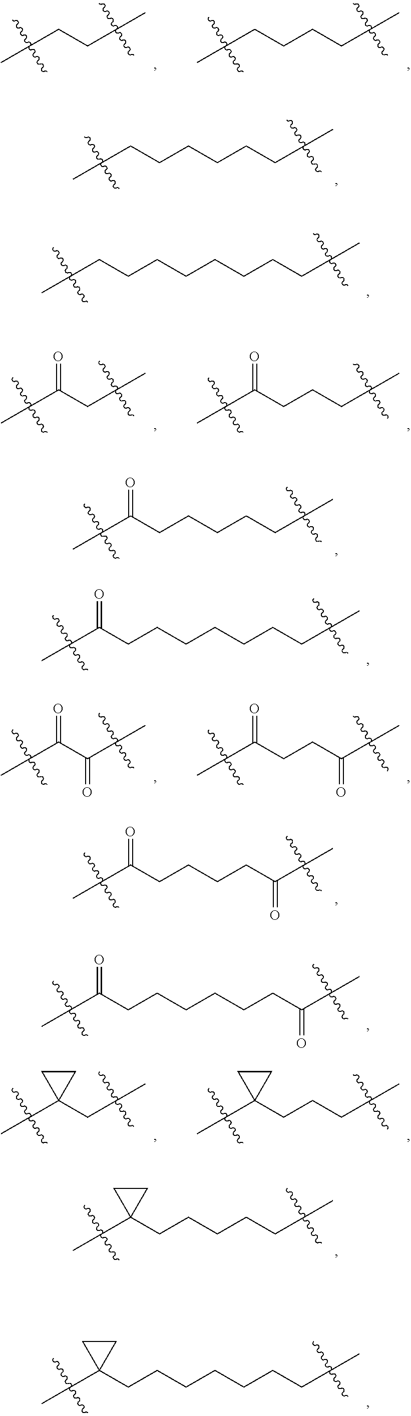

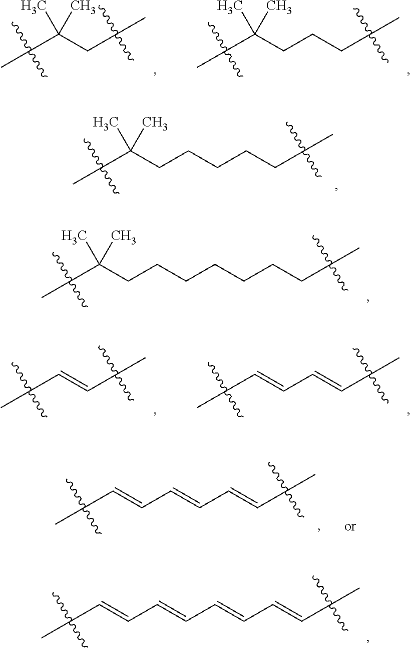

In some variations of formula (33) described in the paragraphs above, L1 is linker (c.1). In some variations of formula (33) described in the paragraphs above, L2 is linker (c.1). In some variations of formula (33) described in the paragraphs above, L3 is linker (c.1). In some variations of formula (33) described in the paragraphs above, L4 is linker (c.1). In some variations of formula (33) described in the paragraphs above, L1 and L2 are linker (c.1). In some variations of formula (33) described in the paragraphs above, L1 and L3 are linker (c.1). In some variations of formula (33) described in the paragraphs above, L1 and L4 are linker (c.1). In some variations of formula (33) described in the paragraphs above, L2 and L3 are linker (c.1). In some variations of formula (33) described in the paragraphs above, L2 and L4 are linker (c.1). In some variations of formula (33) described in the paragraphs above, L3 and L4 are linker (c.1). In some variations of formula (33) described in the paragraphs above, L1, L2, and L3 are linker (c.1). In some variations of formula (33) described in the paragraphs above, L1, L2, and L4 are linker (c.1). In some variations of formula (33) described in the paragraphs above, L1, L3, and L4 are linker (c.1). In some variations of formula (33) described in the paragraphs above, L2, L3, and L4 are linker (c.1). In some variations of formula (33) described in the paragraphs above, L1, L2, L3, and L4 are linker (c.1).

In some variations of formula (33) described in the paragraphs above, at least one of L1, L2, L3, and L4 is linker (c.2):

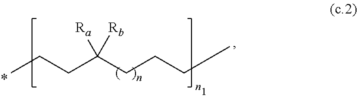

in which n is 0-6, n₁ is 1-4, either (1) Rₐ and R_{b} independently are H or CH₃ or (2) Rₐ and R_{b} are

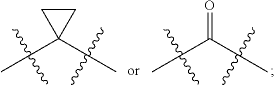

or (3) two of CRₐR_{b} are

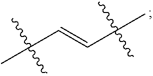 [SMILES: ";" ; */C=C/*]

and * is the attachment site for the cationic dye moiety D.

In some variations of formula (33) in which at least one of L1, L2, L3, and L4 is linker (c.2), n is 0-6, 0-5, 0-4, 0-3, 0-2, 0-1, 1-6, 1-5, 1-4, 1-3, 1-2, 2-6, 2-5, 2-4, 2-3, 3-6, 3-5, 3-4, 4-6, 4-5, 5-6, 0, 1, 2, 3, 4, 5, or 6.

In some variations of formula (33) described in the paragraphs above in which at least one of L1, L2, L3, and L4 is linker (c.2), n₁ is 1-4, 1-3, 1-2, 2-4, 2-3, 3-4, 1, 2, 3, or 4.

In some variations of formula (33) described in the paragraphs above in which at least one of L1, L2, L3, and L4 is linker (c.2), Rₐ is H and R_{b} is H. In some variations of formula (33) described in the paragraphs above in which at least one of L1, L2, L3, and L4 is linker (c.2), Rₐ is H and R_{b} is CH₃. In some variations of formula (33) described in the paragraphs above in which at least one of L1, L2, L3, and L4 is linker (c.2), Rₐ and R_{b} are

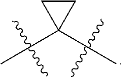 [SMILES: *C1(*)CC1 ; "."]

In some variations of formula (33) described in the paragraphs above in which at least one of L1, L2, L3, and L4 is linker (c.2), Rₐ and R_{b} are

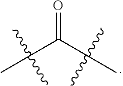 [SMILES: *C(*)=O ; "."]

In some variations of formula (33) described in the paragraphs above in which at least one of L1, L2, L3, and L4 is linker (c.2), two of CRₐR_{b} are

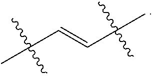 [SMILES: "." ; */C=C/*]

In some variations of formula (33) described in the paragraphs above in which at least one of L1, L2, L3, and L4 is linker (c.2), L is

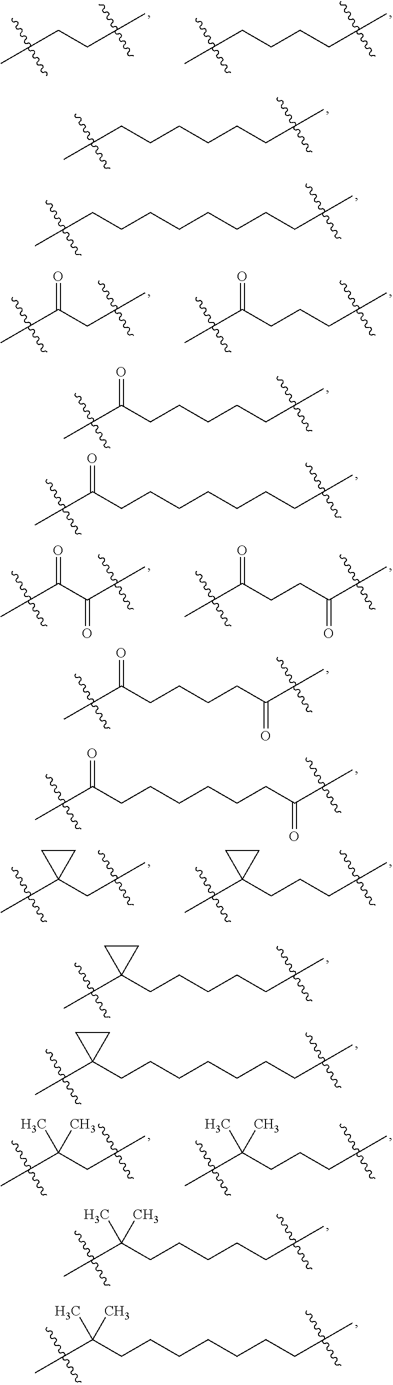

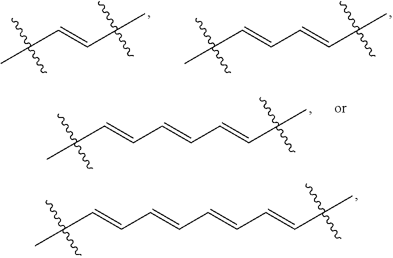

In some variations of formula (33) described in the paragraphs above, L1 is linker (c.2). In some variations of formula (33) described in the paragraphs above, L2 is linker (c.2). In some variations of formula (33) described in the paragraphs above, L3 is linker (c.2). In some variations of formula (33) described in the paragraphs above, L4 is linker (c.2). In some variations of formula (33) described in the paragraphs above, L1 and L2 are linker (c.2). In some variations of formula (33) described in the paragraphs above, L1 and L3 are linker (c.2). In some variations of formula (33) described in the paragraphs above, L1 and L4 are linker (c.2). In some variations of formula (33) described in the paragraphs above, L2 and L3 are linker (c.2). In some variations of formula (33) described in the paragraphs above, L2 and L4 are linker (c.2). In some variations of formula (33) described in the paragraphs above, L3 and L4 are linker (c.2). In some variations of formula (33) described in the paragraphs above, L1, L2, and L3 are linker (c.2). In some variations of formula (33) described in the paragraphs above, L1, L2, and L4 are linker (c.2). In some variations of formula (33) described in the paragraphs above, L1, L3, and L4 are linker (c.2). In some variations of formula (33) described in the paragraphs above, L2, L3, and L4 are linker (c.2). In some variations of formula (33) described in the paragraphs above, L1, L2, L3, and L4 are linker (c.2).

In some variations of formula (33) described in the paragraphs above, at least one of L1, L2, L3, and L4 is linker (d):

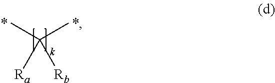

where k is 2-10; (1) Rₐ and R_{b} independently are H or CH₃, or (2) Rₐ and R_{b} are

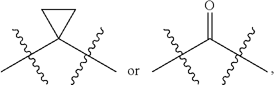

or (3) two of CRₐR_{b} are

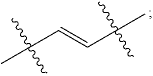 [SMILES: ";" ; */C=C/*]

and * is the attachment site for the cationic dye moiety D.

In some variations of formula (33) in which at least one of L1, L2, L3, and L4 is linker (d), k is 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, 9-10, 2, 3, 4, 5, 6, 7, 8, 9, or 10.

In some variations of formula (33) described in the paragraphs above in which at least one of L1, L2, L3, and L4 is linker (d), Rₐ is H and R_{b} is H. In some variations of formula (33) described in the paragraphs above in which at least one of L1, L2, L3, and L4 is linker (d), Rₐ is H and R_{b} is CH₃. In some variations of formula (33) described in the paragraphs above in which at least one of L1, L2, L3, and L4 is linker (d), Rₐ and R_{b} are

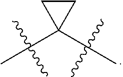 [SMILES: *C1(*)CC1 ; "."]

In some variations of formula (33) described in the paragraphs above in which at least one of L1, L2, L3, and L4 is linker (d), Rₐ and R_{b} are

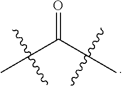 [SMILES: *C(*)=O ; "."]

In some variations of formula (33) described in the paragraphs above in which at least one of L1, L2, L3, and L4 is linker (d), two of CRₐR_{b} are

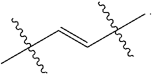 [SMILES: "." ; */C=C/*]

In some variations of formula (33) described in the paragraphs above, L1 is linker (d). In some variations of formula (33) described in the paragraphs above, L2 is linker (d). In some variations of formula (33) described in the paragraphs above, L3 is linker (d). In some variations of formula (33) described in the paragraphs above, L4 is linker (d). In some variations of formula (33) described in the paragraphs above, L1 and L2 are linker (d). In some variations of formula (33) described in the paragraphs above, L1 and L3 are linker (d). In some variations of formula (33) described in the paragraphs above, L1 and L4 are linker (d). In some variations of formula (33) described in the paragraphs above, L2 and L3 are linker (d). In some variations of formula (33) described in the paragraphs above, L2 and L4 are linker (d). In some variations of formula (33) described in the paragraphs above, L3 and L4 are linker (d). In some variations of formula (33) described in the paragraphs above, L1, L2, and L3 are linker (d). In some variations of formula (33) described in the paragraphs above, L1, L2, and L4 are linker (d). In some variations of formula (33) described in the paragraphs above, L1, L3, and L4 are linker (d). In some variations of formula (33) described in the paragraphs above, L2, L3, and L4 are linker (d). In some variations of formula (33) described in the paragraphs above, L1, L2, L3, and L4 are linker (d).

In some variations of formula (33) described in the paragraphs above, at least one of L1, L2, L3, and L4 is linker (e.1):

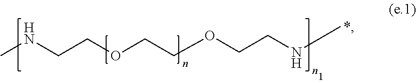

in which n is 0-6, n₁ is 1-4, and * is the attachment site for the cationic dye moiety D.

In some variations of formula (33) in which at least one of L1, L2, L3, and L4 is linker (e.1), n is 0-6, 0-5, 0-4, 0-3, 0-2, 0-1, 1-6, 1-5, 1-4, 1-3, 1-2, 2-6, 2-5, 2-4, 2-3, 3-6, 3-5, 3-4, 4-6, 4-5, 5-6, 0, 1, 2, 3, 4, 5, or 6.

In some variations of formula (33) described in the paragraphs above in which at least one of L1, L2, L3, and L4 is linker (e.1), n₁ is 1-4, 1-3, 1-2, 2-4, 2-3, 3-4, 1, 2, 3, or 4.

In some variations of formula (33) described in the paragraphs above, L1 is linker (e.1). In some variations of formula (33) described in the paragraphs above, L2 is linker (e.1). In some variations of formula (33) described in the paragraphs above, L3 is linker (e.1). In some variations of formula (33) described in the paragraphs above, L4 is linker (e.1). In some variations of formula (33) described in the paragraphs above, L1 and L2 are linker (e.1). In some variations of formula (33) described in the paragraphs above, L1 and L3 are linker (e.1). In some variations of formula (33) described in the paragraphs above, L1 and L4 are linker (e.1). In some variations of formula (33) described in the paragraphs above, L2 and L3 are linker (e.1). In some variations of formula (33) described in the paragraphs above, L2 and L4 are linker (e.1). In some variations of formula (33) described in the paragraphs above, L3 and L4 are linker (e.1). In some variations of formula (33) described in the paragraphs above, L1, L2, and L3 are linker (e.1). In some variations of formula (33) described in the paragraphs above, L1, L2, and L4 are linker (e.1). In some variations of formula (33) described in the paragraphs above, L1, L3, and L4 are linker (e.1). In some variations of formula (33) described in the paragraphs above, L2, L3, and L4 are linker (e.1). In some variations of formula (33) described in the paragraphs above, L1, L2, L3, and L4 are linker (e.1).

In some variations of formula (33) described in the paragraphs above, at least one of L1, L2, L3, and L4 is linker (f.1):

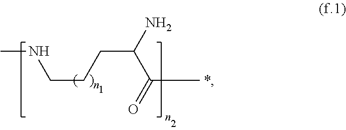

in which n₁ is 0-5, n₂ is 1-5, and * is the attachment site for the cationic dye moiety D.

In some variations of formula (33) in which at least one of L1, L2, L3, and L4 is linker (f.1), n₁ is 0-5, 0-4, 0-3, 0-2, 0-1, 1-5, 1-4, 1-3, 1-2, 2-5, 2-4, 2-3, 3-5, 3-4, 4-5, 0, 1, 2, 3, 4, or 5.

In some variations of formula (33) described in the paragraphs above in which at least one of L1, L2, L3, and L4 is linker (f.1), n₂ is 1-5, 1-4, 1-3, 1-2, 2-5, 2-4, 2-3, 3-5, 3-4, 4-5, 1, 2, 3, 4, or 5.

In some variations of formula (33) described in the paragraphs above, L1 is linker (f.1). In some variations of formula (33) described in the paragraphs above, L2 is linker (f.1). In some variations of formula (33) described in the paragraphs above, L3 is linker (f.1). In some variations of formula (33) described in the paragraphs above, L4 is linker (f.1). In some variations of formula (33) described in the paragraphs above, L1 and L2 are linker (f.1). In some variations of formula (33) described in the paragraphs above, L1 and L3 are linker (f.1). In some variations of formula (33) described in the paragraphs above, L1 and L4 are linker (f.1). In some variations of formula (33) described in the paragraphs above, L2 and L3 are linker (f.1). In some variations of formula (33) described in the paragraphs above, L2 and L4 are linker (f.1). In some variations of formula (33) described in the paragraphs above, L3 and L4 are linker (f.1). In some variations of formula (33) described in the paragraphs above, L1, L2, and L3 are linker (f.1). In some variations of formula (33) described in the paragraphs above, L1, L2, and L4 are linker (f.1). In some variations of formula (33) described in the paragraphs above, L1, L3, and L4 are linker (f.1). In some variations of formula (33) described in the paragraphs above, L2, L3, and L4 are linker (f.1). In some variations of formula (33) described in the paragraphs above, L1, L2, L3, and L4 are linker (f.1).

In some variations of formula (33) described in the paragraphs above, at least one of L1, L2, L3, and L4 is linker (f.2):

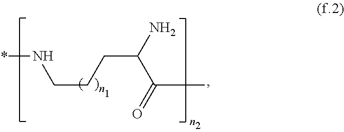

in which n₁ is 0-5, n₂ is 1-5, and * is the attachment site for the cationic dye moiety D.

In some variations of formula (33) in which at least one of L1, L2, L3, and L4 is linker (f.2), n₁ is 0-5, 0-4, 0-3, 0-2, 0-1, 1-5, 1-4, 1-3, 1-2, 2-5, 2-4, 2-3, 3-5, 3-4, 4-5, 0, 1, 2, 3, 4, or 5.

In some variations of formula (33) described in the paragraphs above in which at least one of L1, L2, L3, and L4 is linker (f.2), n₂ is 1-5, 1-4, 1-3, 1-2, 2-5, 2-4, 2-3, 3-5, 3-4, 4-5, 1, 2, 3, 4, or 5.

In some variations of formula (33) described in the paragraphs above, L1 is linker (f.2). In some variations of formula (33) described in the paragraphs above, L2 is linker (f.2). In some variations of formula (33) described in the paragraphs above, L3 is linker (f.2). In some variations of formula (33) described in the paragraphs above, L4 is linker (f.2). In some variations of formula (33) described in the paragraphs above, L1 and L2 are linker (f.2). In some variations of formula (33) described in the paragraphs above, L1 and L3 are linker (f.2). In some variations of formula (33) described in the paragraphs above, L1 and L4 are linker (f.2). In some variations of formula (33) described in the paragraphs above, L2 and L3 are linker (f.2). In some variations of formula (33) described in the paragraphs above, L2 and L4 are linker (f.2). In some variations of formula (33) described in the paragraphs above, L3 and L4 are linker (f.2). In some variations of formula (33) described in the paragraphs above, L1, L2, and L3 are linker (f.2). In some variations of formula (33) described in the paragraphs above, L1, L2, and L4 are linker (f.2). In some variations of formula (33) described in the paragraphs above, L1, L3, and L4 are linker (f.2). In some variations of formula (33) described in the paragraphs above, L2, L3, and L4 are linker (f.2). In some variations of formula (33) described in the paragraphs above, L1, L2, L3, and L4 are linker (f.2).

In some variations of formula (33) described in the paragraphs above, at least one of L1, L2, L3, and L4 is linker (g.1):

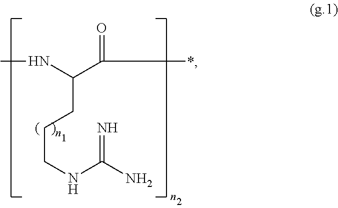

in which n₁ is 0-5, n₂ is 1-5, and * is the attachment site for the cationic dye moiety D.

In some variations of formula (33) in which at least one of L1, L2, L3, and L4 is linker (g.1), n₁ is 0-5, 0-4, 0-3, 0-2, 0-1, 1-5, 1-4, 1-3, 1-2, 2-5, 2-4, 2-3, 3-5, 3-4, 4-5, 0, 1, 2, 3, 4, or 5.

In some variations of formula (33) described in the paragraphs above in which at least one of L1, L2, L3, and L4 is linker (g.1), n₂ is 1-5, 1-4, 1-3, 1-2, 2-5, 2-4, 2-3, 3-5, 3-4, 4-5, 1, 2, 3, 4, or 5.

In some variations of formula (33) described in the paragraphs above, L1 is linker (g.1). In some variations of formula (33) described in the paragraphs above, L2 is linker (g.1). In some variations of formula (33) described in the paragraphs above, L3 is linker (g.1). In some variations of formula (33) described in the paragraphs above, L4 is linker (g.1). In some variations of formula (33) described in the paragraphs above, L1 and L2 are linker (g.1). In some variations of formula (33) described in the paragraphs above, L1 and L3 are linker (g.1). In some variations of formula (33) described in the paragraphs above, L1 and L4 are linker (g.1). In some variations of formula (33) described in the paragraphs above, L2 and L3 are linker (g.1). In some variations of formula (33) described in the paragraphs above, L2 and L4 are linker (g.1). In some variations of formula (33) described in the paragraphs above, L3 and L4 are linker (g.1). In some variations of formula (33) described in the paragraphs above, L1, L2, and L3 are linker (g.1). In some variations of formula (33) described in the paragraphs above, L1, L2, and L4 are linker (g.1). In some variations of formula (33) described in the paragraphs above, L1, L3, and L4 are linker (g.1). In some variations of formula (33) described in the paragraphs above, L2, L3, and L4 are linker (g.1). In some variations of formula (33) described in the paragraphs above, L1, L2, L3, and L4 are linker (g.1).

In some variations of formula (33) described in the paragraphs above, at least one of L1, L2, L3, and L4 is linker (g.2):

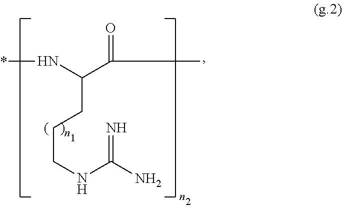

in which n₁ is 0-5, n₂ is 1-5, and * is the attachment site for the cationic dye moiety D.

In some variations of formula (33) in which at least one of L1, L2, L3, and L4 is linker (g.2), n₁ is 0-5, 0-4, 0-3, 0-2, 0-1, 1-5, 1-4, 1-3, 1-2, 2-5, 2-4, 2-3, 3-5, 3-4, 4-5, 0, 1, 2, 3, 4, or 5.

In some variations of formula (33) described in the paragraphs above in which at least one of L1, L2, L3, and L4 is linker (g.2), n₂ is 1-5, 1-4, 1-3, 1-2, 2-5, 2-4, 2-3, 3-5, 3-4, 4-5, 1, 2, 3, 4, or 5.

In some variations of formula (33) described in the paragraphs above, L1 is linker (g.2). In some variations of formula (33) described in the paragraphs above, L2 is linker (g.2). In some variations of formula (33) described in the paragraphs above, L3 is linker (g.2). In some variations of formula (33) described in the paragraphs above, L4 is linker (g.2). In some variations of formula (33) described in the paragraphs above, L1 and L2 are linker (g.2). In some variations of formula (33) described in the paragraphs above, L1 and L3 are linker (g.2). In some variations of formula (33) described in the paragraphs above, L1 and L4 are linker (g.2). In some variations of formula (33) described in the paragraphs above, L2 and L3 are linker (g.2). In some variations of formula (33) described in the paragraphs above, L2 and L4 are linker (g.2). In some variations of formula (33) described in the paragraphs above, L3 and L4 are linker (g.2). In some variations of formula (33) described in the paragraphs above, L1, L2, and L3 are linker (g.2). In some variations of formula (33) described in the paragraphs above, L1, L2, and L4 are linker (g.2). In some variations of formula (33) described in the paragraphs above, L1, L3, and L4 are linker (g.2). In some variations of formula (33) described in the paragraphs above, L2, L3, and L4 are linker (g.2). In some variations of formula (33) described in the paragraphs above, L1, L2, L3, and L4 are linker (g.2).

In some variations of formula (33) described in the paragraphs above, at least one of L1, L2, L3, and L4 is linker (h.1):

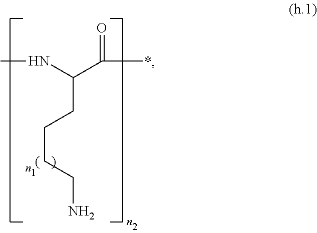

in which n₁ is 0-5, n₂ is 1-5, and * is the attachment site for the cationic dye moiety D.

In some variations of formula (33) in which at least one of L1, L2, L3, and L4 is linker (h.1), n₁ is 0-5, 0-4, 0-3, 0-2, 0-1, 1-5, 1-4, 1-3, 1-2, 2-5, 2-4, 2-3, 3-5, 3-4, 4-5, 0, 1, 2, 3, 4, or 5.

In some variations of formula (33) described in the paragraphs above in which at least one of L1, L2, L3, and L4 is linker (h.1), n₂ is 1-5, 1-4, 1-3, 1-2, 2-5, 2-4, 2-3, 3-5, 3-4, 4-5, 1, 2, 3, 4, or 5.

In some variations of formula (33) described in the paragraphs above, L1 is linker (h.1). In some variations of formula (33) described in the paragraphs above, L2 is linker (h.1). In some variations of formula (33) described in the paragraphs above, L3 is linker (h.1). In some variations of formula (33) described in the paragraphs above, L4 is linker (h.1). In some variations of formula (33) described in the paragraphs above, L1 and L2 are linker (h.1). In some variations of formula (33) described in the paragraphs above, L1 and L3 are linker (h.1). In some variations of formula (33) described in the paragraphs above, L1 and L4 are linker (h.1). In some variations of formula (33) described in the paragraphs above, L2 and L3 are linker (h.1). In some variations of formula (33) described in the paragraphs above, L2 and L4 are linker (h.1). In some variations of formula (33) described in the paragraphs above, L3 and L4 are linker (h.1). In some variations of formula (33) described in the paragraphs above, L1, L2, and L3 are linker (h.1). In some variations of formula (33) described in the paragraphs above, L1, L2, and L4 are linker (h.1). In some variations of formula (33) described in the paragraphs above, L1, L3, and L4 are linker (h.1). In some variations of formula (33) described in the paragraphs above, L2, L3, and L4 are linker (h.1). In some variations of formula (33) described in the paragraphs above, L1, L2, L3, and L4 are linker (h.1).

In some variations of formula (33) described in the paragraphs above, at least one of L1, L2, L3, and L4 is linker (h.2):

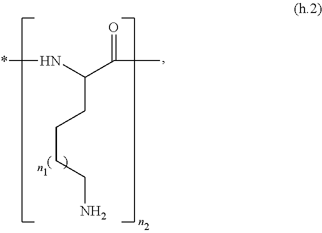

in which n₁ is 0-5, n₂ is 1-5, and * is the attachment site for the cationic dye moiety D.

In some variations of formula (33) in which at least one of L1, L2, L3, and L4 is linker (h.2), n₁ is 0-5, 0-4, 0-3, 0-2, 0-1, 1-5, 1-4, 1-3, 1-2, 2-5, 2-4, 2-3, 3-5, 3-4, 4-5, 0, 1, 2, 3, 4, or 5.

In some variations of formula (33) described in the paragraphs above in which at least one of L1, L2, L3, and L4 is linker (h.2), n₂ is 1-5, 1-4, 1-3, 1-2, 2-5, 2-4, 2-3, 3-5, 3-4, 4-5, 1, 2, 3, 4, or 5.

In some variations of formula (33) described in the paragraphs above, L1 is linker (h.2). In some variations of formula (33) described in the paragraphs above, L2 is linker (h.2). In some variations of formula (33) described in the paragraphs above, L3 is linker (h.2). In some variations of formula (33) described in the paragraphs above, L4 is linker (h.2). In some variations of formula (33) described in the paragraphs above, L1 and L2 are linker (h.2). In some variations of formula (33) described in the paragraphs above, L1 and L3 are linker (h.2). In some variations of formula (33) described in the paragraphs above, L1 and L4 are linker (h.2). In some variations of formula (33) described in the paragraphs above, L2 and L3 are linker (h.2). In some variations of formula (33) described in the paragraphs above, L2 and L4 are linker (h.2). In some variations of formula (33) described in the paragraphs above, L3 and L4 are linker (h.2). In some variations of formula (33) described in the paragraphs above, L1, L2, and L3 are linker (h.2). In some variations of formula (33) described in the paragraphs above, L1, L2, and L4 are linker (h.2). In some variations of formula (33) described in the paragraphs above, L1, L3, and L4 are linker (h.2). In some variations of formula (33) described in the paragraphs above, L2, L3, and L4 are linker (h.2). In some variations of formula (33) described in the paragraphs above, L1, L2, L3, and L4 are linker (h.2).

In some variations of formula (33) described in the paragraphs above, at least one of L1, L2, L3, and L4 is linker (i.1):

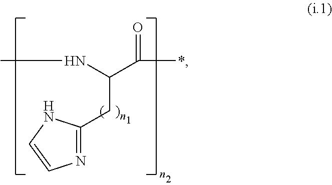

in which n₁ and n₂ independently are 1-5 and * is the attachment site for the cationic dye moiety D.

In some variations of formula (33) described in the paragraphs above, at least one of L1, L2, L3, and L4 is linker (i.1), n₁ is 1-5, 1-4, 1-3, 1-2, 2-5, 2-4, 2-3, 3-5, 3-4, 4-5, 1, 2, 3, 4, or 5.

In some variations of formula (33) described in the paragraphs above in which at least one of L1, L2, L3, and L4 is linker (i.1), n₁ is 1-5, 1-4, 1-3, 1-2, 2-5, 2-4, 2-3, 3-5, 3-4, 4-5, 1, 2, 3, 4, or 5.

In some variations of formula (33) described in the paragraphs above, L1 is linker (i.1). In some variations of formula (33) described in the paragraphs above, L2 is linker (i.1). In some variations of formula (33) described in the paragraphs above, L3 is linker (i.1). In some variations of formula (33) described in the paragraphs above, L4 is linker (i.1). In some variations of formula (33) described in the paragraphs above, L1 and L2 are linker (i.1). In some variations of formula (33) described in the paragraphs above, L1 and L3 are linker (i.1). In some variations of formula (33) described in the paragraphs above, L1 and L4 are linker (i.1). In some variations of formula (33) described in the paragraphs above, L2 and L3 are linker (i.1). In some variations of formula (33) described in the paragraphs above, L2 and L4 are linker (i.1). In some variations of formula (33) described in the paragraphs above, L3 and L4 are linker (i.1). In some variations of formula (33) described in the paragraphs above, L1, L2, and L3 are linker (i.1). In some variations of formula (33) described in the paragraphs above, L1, L2, and L4 are linker (i.1). In some variations of formula (33) described in the paragraphs above, L1, L3, and L4 are linker (i.1). In some variations of formula (33) described in the paragraphs above, L2, L3, and L4 are linker (i.1). In some variations of formula (33) described in the paragraphs above, L1, L2, L3, and L4 are linker (i.1).

In some variations of formula (33) described in the paragraphs above, at least one of L1, L2, L3, and L4 is linker (i.2):

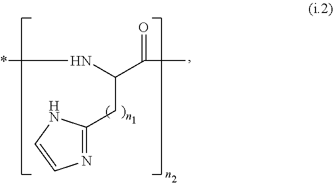

in which n₁ and n₂ independently are 1-5 and * is the attachment site for the cationic dye moiety D.

In some variations of formula (33) described in the paragraphs above, at least one of L1, L2, L3, and L4 is linker (i.2), n₁ is 1-5, 1-4, 1-3, 1-2, 2-5, 2-4, 2-3, 3-5, 3-4, 4-5, 1, 2, 3, 4, or 5.

In some variations of formula (33) described in the paragraphs above in which at least one of L1, L2, L3, and L4 is linker (i.2), n₁ is 1-5, 1-4, 1-3, 1-2, 2-5, 2-4, 2-3, 3-5, 3-4, 4-5, 1, 2, 3, 4, or 5.

In some variations of formula (33) described in the paragraphs above, L1 is linker (i.2). In some variations of formula (33) described in the paragraphs above, L2 is linker (i.2). In some variations of formula (33) described in the paragraphs above, L3 is linker (i.2). In some variations of formula (33) described in the paragraphs above, L4 is linker (i.2). In some variations of formula (33) described in the paragraphs above, L1 and L2 are linker (i.2). In some variations of formula (33) described in the paragraphs above, L1 and L3 are linker (i.2). In some variations of formula (33) described in the paragraphs above, L1 and L4 are linker (i.2). In some variations of formula (33) described in the paragraphs above, L2 and L3 are linker (i.2). In some variations of formula (33) described in the paragraphs above, L2 and L4 are linker (i.2). In some variations of formula (33) described in the paragraphs above, L3 and L4 are linker (i.2). In some variations of formula (33) described in the paragraphs above, L1, L2, and L3 are linker (i.2). In some variations of formula (33) described in the paragraphs above, L1, L2, and L4 are linker (i.2). In some variations of formula (33) described in the paragraphs above, L1, L3, and L4 are linker (i.2). In some variations of formula (33) described in the paragraphs above, L2, L3, and L4 are linker (i.2). In some variations of formula (33) described in the paragraphs above, L1, L2, L3, and L4 are linker (i.2).

In some variations of formula (33) described in the paragraphs above, at least one of L1, L2, L3, and L4 is linker (j.1):

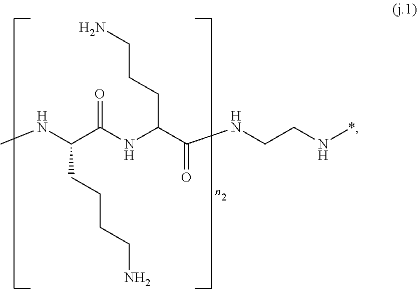

in which n₂ is 1-5 and * is the attachment site for the cationic dye moiety D.

In some variations of formula (33) in which at least one of L1, L2, L3, and L4 is linker (j.1), n₂ is 1-5, 1-4, 1-3, 1-2, 2-5, 2-4, 2-3, 3-5, 3-4, 4-5, 1, 2, 3, 4, or 5.

In some variations of formula (33) described in the paragraphs above, L1 is linker (j.1). In some variations of formula (33) described in the paragraphs above, L2 is linker (j.1). In some variations of formula (33) described in the paragraphs above, L3 is linker (j.1). In some variations of formula (33) described in the paragraphs above, L4 is linker (j.1). In some variations of formula (33) described in the paragraphs above, L1 and L2 are linker (j.1). In some variations of formula (33) described in the paragraphs above, L1 and L3 are linker (j.1). In some variations of formula (33) described in the paragraphs above, L1 and L4 are linker (j.1). In some variations of formula (33) described in the paragraphs above, L2 and L3 are linker (j.1). In some variations of formula (33) described in the paragraphs above, L2 and L4 are linker (j.1). In some variations of formula (33) described in the paragraphs above, L3 and L4 are linker (j.1). In some variations of formula (33) described in the paragraphs above, L1, L2, and L3 are linker (j.1). In some variations of formula (33) described in the paragraphs above, L1, L2, and L4 are linker (j.1). In some variations of formula (33) described in the paragraphs above, L1, L3, and L4 are linker (j.1). In some variations of formula (33) described in the paragraphs above, L2, L3, and L4 are linker (j.1). In some variations of formula (33) described in the paragraphs above, L1, L2, L3, and L4 are linker (j.1).

In some variations of formula (33) described in the paragraphs above, at least one of L1, L2, L3, and L4 is linker (j.2):

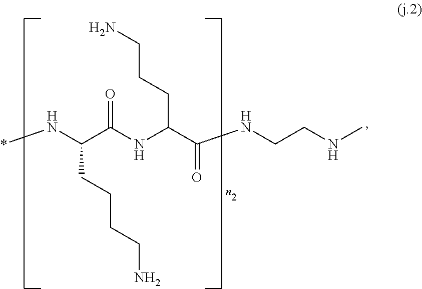

in which n₂ is 1-5 and * is the attachment site for the cationic dye moiety D.

In some variations of formula (33) in which at least one of L1, L2, L3, and L4 is linker (j.2), n₂ is 1-5, 1-4, 1-3, 1-2, 2-5, 2-4, 2-3, 3-5, 3-4, 4-5, 1, 2, 3, 4, or 5.

In some variations of formula (33) described in the paragraphs above, L1 is linker (j.2). In some variations of formula (33) described in the paragraphs above, L2 is linker (j.2). In some variations of formula (33) described in the paragraphs above, L3 is linker (j.2). In some variations of formula (33) described in the paragraphs above, L4 is linker (j.2). In some variations of formula (33) described in the paragraphs above, L1 and L2 are linker (j.2). In some variations of formula (33) described in the paragraphs above, L1 and L3 are linker (j.2). In some variations of formula (33) described in the paragraphs above, L1 and L4 are linker (j.2). In some variations of formula (33) described in the paragraphs above, L2 and L3 are linker (j.2). In some variations of formula (33) described in the paragraphs above, L2 and L4 are linker (j.2). In some variations of formula (33) described in the paragraphs above, L3 and L4 are linker (j.2). In some variations of formula (33) described in the paragraphs above, L1, L2, and L3 are linker (j.2). In some variations of formula (33) described in the paragraphs above, L1, L2, and L4 are linker (j.2). In some variations of formula (33) described in the paragraphs above, L1, L3, and L4 are linker (j.2). In some variations of formula (33) described in the paragraphs above, L2, L3, and L4 are linker (j.2). In some variations of formula (33) described in the paragraphs above, L1, L2, L3, and L4 are linker (j.2).

In some variations of formula (33) described in the paragraphs above, at least one of L1, L2, L3, and L4 is linker (k):

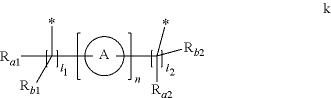

in which l₁ and l₂ independently are 1-4, n is 1-4; ring A is aryl, heteroaryl, cycloalkyl, or heterocyclyl; (1) Rₐ₁ and R_{b1} independently are H or CH₃ or (2) Rₐ₁ and R_{b1} independently are

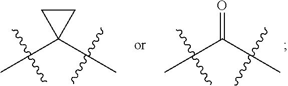

or (3) two of CRₐ₁R_{b1} are

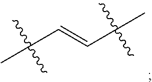 [SMILES: */C=C/*]

(1) Rₐ₂ and R_{b2} independently are H or CH₃ or (2) Rₐ₂ and R_{b2} independently are

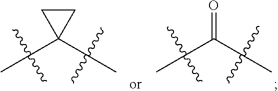

or (3) two of CRₐ₂R_{b2} are

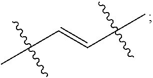 [SMILES: */C=C/*]

and * is the attachment site for the cationic dye moiety D.

In some variations of formula (33) in which at least one of L1, L2, L3, and L4 is linker (k), l₁ is 1-4, 1-3, 1-3, 1-2, 2-4, 2-3, 3-4, 1, 2, 3, or 4.

In some variations of formula (33) described in the paragraphs above in which at least one of L1, L2, L3, and L4 is linker (k), l₂ is 1-4, 1-3, 1-3, 1-2, 2-4, 2-3, 3-4, 1, 2, 3, or 4.

In some variations of formula (33) described in the paragraphs above in which at least one of L1, L2, L3, and L4 is linker (k), Rₐ₁ is H and R_{b1} is H. In some variations of formula (33) described in the paragraphs above in which at least one of L1, L2, L3, and L4 is linker (k), Rₐ₁ is H and R_{b1} is CH₃. In some variations of formula (33) described in the paragraphs above in which at least one of L1, L2, L3, and L4 is linker (k), Rₐ₁ and R_{b1} are

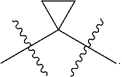 [SMILES: *C1(*)CC1]

In some variations of formula (33) described in the paragraphs above in which at least one of L1, L2, L3, and L4 is linker (k), Rₐ₁ and R_{b1} are

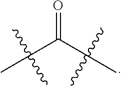 [SMILES: *C(*)=O]

In some variations of formula (33) described in the paragraphs above in which at least one of L1, L2, L3, and L4 is linker (k), two of CRₐ₁R_{b1} are

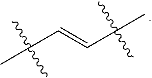 [SMILES: */C=C/*]

In some variations of formula (33) described in the paragraphs above in which at least one of L1, L2, L3, and L4 is linker (k), Rₐ₂ is H and R_{b2} is H. In some variations of formula (33) described in the paragraphs above in which at least one of L1, L2, L3, and L4 is linker (k), Rₐ₂ is H and R_{b2} is CH₃. In some variations of formula (33) described in the paragraphs above in which at least one of L1, L2, L3, and L4 is linker (k), Rₐ₂ and R_{b2} are

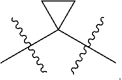 [SMILES: *C1(*)CC1]

In some variations of formula (33) described in the paragraphs above in which at least one of L1, L2, L3, and L4 is linker (k), Rₐ₂ and R_{b2} are

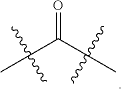 [SMILES: *C(*)=O]

In some variations of formula (33) described in the paragraphs above in which at least one of L1, L2, L3, and L4 is linker (k), two of CRₐ₂R_{b2} are

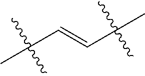 [SMILES: */C=C/*]

In some variations of formula (33) described in the paragraphs above in which at least one of L1, L2, L3, and L4 is linker (k), ring A is

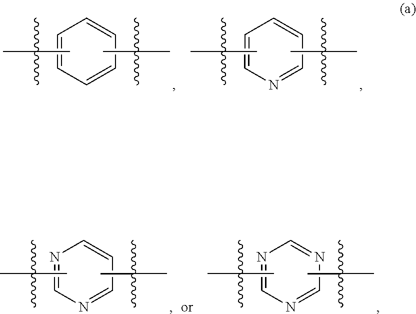

optionally substituted with halo or C1-C6 linear or branched alkyl;

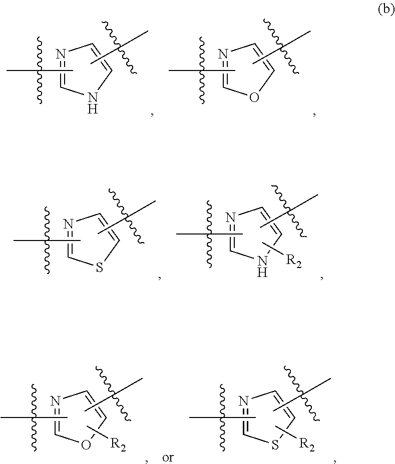

optionally substituted with halo or C1-C6 linear or branched alkyl; or

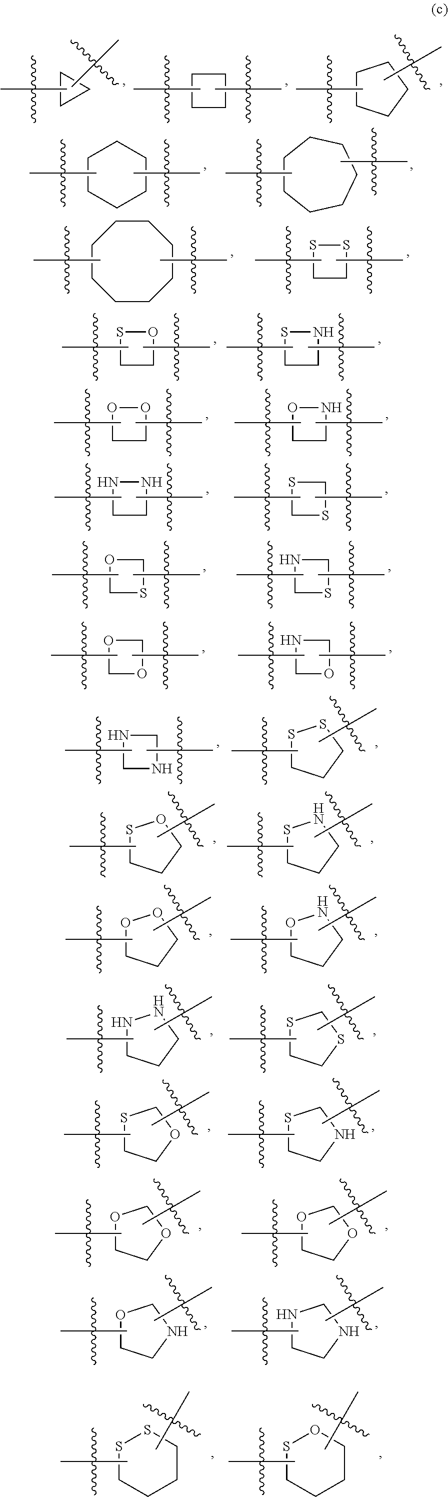

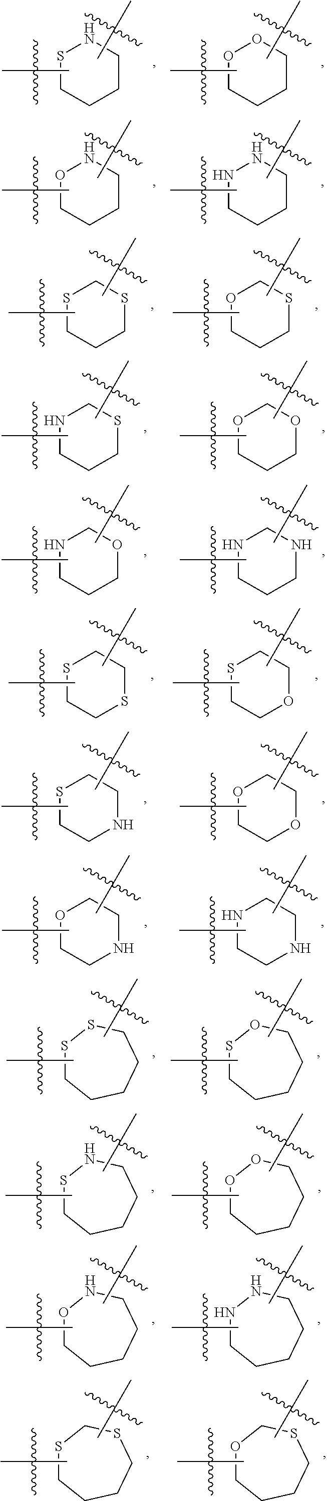

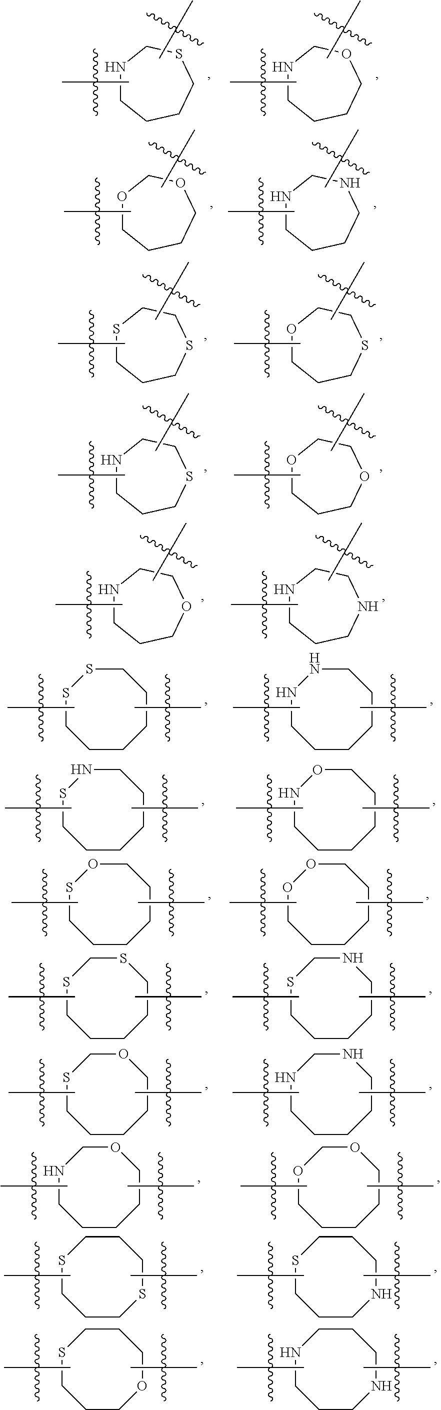

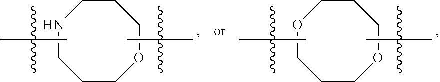

optionally substituted with halo or C1-C6 linear or branched alkyl.

In some variations of formula (33) described in the paragraphs above in which L is linker (k), ring A is substituted with halo. In some variations, the halo is F, Br, I, or Cl.

In some variations of formula (33) described in the paragraphs above in which L is linker (k), ring A is substituted with C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (33) described in the paragraphs above, L1 is linker (k). In some variations of formula (33) described in the paragraphs above, L2 is linker (k). In some variations of formula (33) described in the paragraphs above, L3 is linker (k). In some variations of formula (33) described in the paragraphs above, L4 is linker (k). In some variations of formula (33) described in the paragraphs above, L1 and L2 are linker (k). In some variations of formula (33) described in the paragraphs above, L1 and L3 are linker (k). In some variations of formula (33) described in the paragraphs above, L1 and L4 are linker (k). In some variations of formula (33) described in the paragraphs above, L2 and L3 are linker (k). In some variations of formula (33) described in the paragraphs above, L2 and L4 are linker (k). In some variations of formula (33) described in the paragraphs above, L3 and L4 are linker (k). In some variations of formula (33) described in the paragraphs above, L1, L2, and L3 are linker (k). In some variations of formula (33) described in the paragraphs above, L1, L2, and L4 are linker (k). In some variations of formula (33) described in the paragraphs above, L1, L3, and L4 are linker (k). In some variations of formula (33) described in the paragraphs above, L2, L3, and L4 are linker (k). In some variations of formula (33) described in the paragraphs above, L1, L2, L3, and L4 are linker (k).

In some variations of formula (33) described in the paragraphs above, at least one of L1, L2, L3, and L4 is linker (l.1):

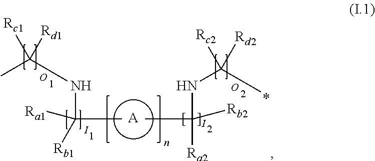

in which l₁, l₂, n, o₁, and o₂ independently are 1-4; ring A is aryl, heteroaryl, cycloalkyl, or heterocyclyl; for each independent instance of Rₐ₁ and R_{b1}, Rₐ₁ and R_{b1} (1) independently are H or CH₃, or (2) Rₐ₁ and R_{b1} independently are

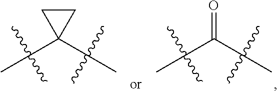

or (3) two of CRₐ₁R_{b1} are

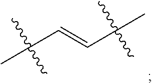 [SMILES: */C=C/*]

for each independent instance of Rₐ₂ and R_{b2}, Rₐ₂ and R_{b2} (1) independently are H or CH₃, or (2) Rₐ₂ and R_{b2} independently are

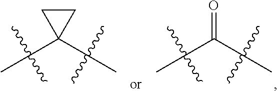

or (3) two of CRₐ₂R_{b2} are

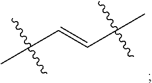 [SMILES: */C=C/*]

for each independent instance of R_{c1} and R_{d1}, R_{c1} and R_{d1} (1) independently are H or CH₃, or (2) R_{c1} and R_{d1} independently are

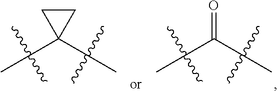

or (3) two of CR_{c1}R_{d1} are

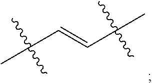 [SMILES: */C=C/*]

for each independent instance of R_{c2} and R_{d2}, R_{c2} and R_{d2} (1) independently are H or CH₃, or (2) R_{c2} and R_{d2} independently are

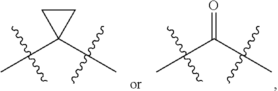

or (3) two of CRₑ₂R_{d2} are

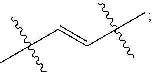 [SMILES: */C=C/*]

and * is the attachment site for the cationic dye moiety D.

In some variations of formula (33) in which at least one of L1, L2, L3, and L4 is linker (l.1), l₁ is 1-4, 1-3, 1-3, 1-2, 2-4, 2-3, 3-4, 1, 2, 3, or 4.

In some variations of formula (33) described in the paragraphs above in which at least one of L1, L2, L3, and L4 is linker (l.1), l₂ is 1-4, 1-3, 1-3, 1-2, 2-4, 2-3, 3-4, 1, 2, 3, or 4.

In some variations of formula (33) described in the paragraphs above in which at least one of L1, L2, L3, and L4 is linker (l.1), o₁ is 1-4, 1-3, 1-3, 1-2, 2-4, 2-3, 3-4, 1, 2, 3, or 4.

In some variations of formula (33) described in the paragraphs above in which at least one of L1, L2, L3, and L4 is linker (l.1), o₂ is 1-4, 1-3, 1-3, 1-2, 2-4, 2-3, 3-4, 1, 2, 3, or 4.

In some variations of formula (33) described in the paragraphs above in which at least one of L1, L2, L3, and L4 is linker (l.1), Rₐ₁ is H and R_{b1} is H. In some variations of formula (33) described in the paragraphs above in which at least one of L1, L2, L3, and L4 is linker (l.1), Rₐ₁ is H and R_{b1} is CH₃. In some variations of formula (33) described in the paragraphs above in which at least one of L1, L2, L3, and L4 is linker (l.1), Rₐ₁ and R_{b1} are

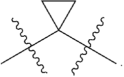 [SMILES: *C1(*)CC1]

In some variations of formula (33) described in the paragraphs above in which at least one of L1, L2, L3, and L4 is linker (l.1), Rₐ₁ and R_{b1} are

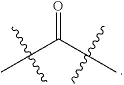 [SMILES: *C(*)=O]

In some variations of formula (33) described in the paragraphs above in which at least one of L1, L2, L3, and L4 is linker (l.1), two of CRₐ₁R_{b1} are

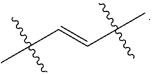 [SMILES: */C=C/*]

In some variations of formula (33) described in the paragraphs above in which at least one of L1, L2, L3, and L4 is linker (l.1), Rₐ₂ is H and R_{b2} is H. In some variations of formula (33) described in the paragraphs above in which at least one of L1, L2, L3, and L4 is linker (l.1), Rₐ₂ is H and R_{b2} is CH₃. In some variations of formula (33) described in the paragraphs above in which at least one of L1, L2, L3, and L4 is linker (l.1), Rₐ₂ and R_{b2} are

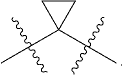 [SMILES: *C1(*)CC1]

In some variations of formula (33) described in the paragraphs above in which at least one of L1, L2, L3, and L4 is linker (l.1), Rₐ₂ and R_{b2} are

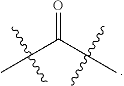 [SMILES: *C(*)=O]

In some variations of formula (33) described in the paragraphs above in which at least one of L1, L2, L3, and L4 is linker (l.1), two of CRₐ₂R_{b2} are

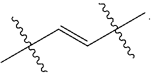 [SMILES: */C=C/*]

In some variations of formula (33) described in the paragraphs above in which at least one of L1, L2, L3, and L4 is linker (l.1), R_{c1} is H and R_{d1} is H. In some variations of formula (33) described in the paragraphs above in which at least one of L1, L2, L3, and L4 is linker (l.1), R_{c1} is H and R_{d1} is CH₃. In some variations of formula (33) described in the paragraphs above in which at least one of L1, L2, L3, and L4 is linker (l.1), R_{c1} and R_{d1} are

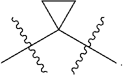 [SMILES: *C1(*)CC1]

In some variations of formula (33) described in the paragraphs above in which at least one of L1, L2, L3, and L4 is linker (l.1), R_{c1} and R_{d1} are

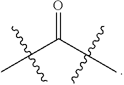 [SMILES: *C(*)=O]

In some variations of formula (33) described in the paragraphs above in which at least one of L1, L2, L3, and L4 is linker (l.1), two of CR_{c1}R_{d1} are

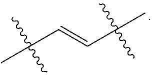 [SMILES: */C=C/*]

In some variations of formula (33) described in the paragraphs above in which at least one of L1, L2, L3, and L4 is linker (l.1), R_{c2} is H and R_{d2} is H. In some variations of formula (33) described in the paragraphs above in which at least one of L1, L2, L3, and L4 is linker (l.1), R_{c2} is H and R_{d2} is CH₃. In some variations of formula (33) described in the paragraphs above in which at least one of L1, L2, L3, and L4 is linker (l.1), R_{c2} and R_{d2} are

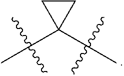 [SMILES: *C1(*)CC1]

In some variations of formula (33) described in the paragraphs above in which at least one of L1, L2, L3, and L4 is linker (l.1), R_{c2} and R_{d2} are

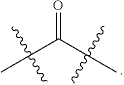 [SMILES: *C(*)=O]

In some variations of formula (33) described in the paragraphs above in which at least one of L1, L2, L3, and L4 is linker (l.1), two of CR_{c2}R_{d2} are

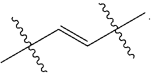 [SMILES: */C=C/*]

In some variations of formula (33) described in the paragraphs above in which at least one of L1, L2, L3, and L4 is linker (l.1), ring A is

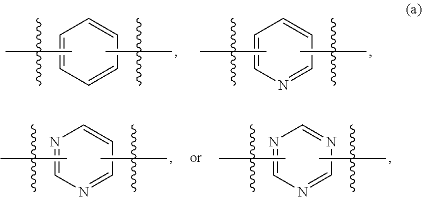

optionally substituted with halo or C1-C6 linear or branched alkyl;

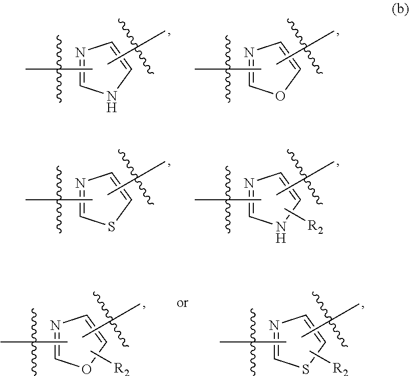

optionally substituted with halo or C1-C6 linear or branched alkyl; or

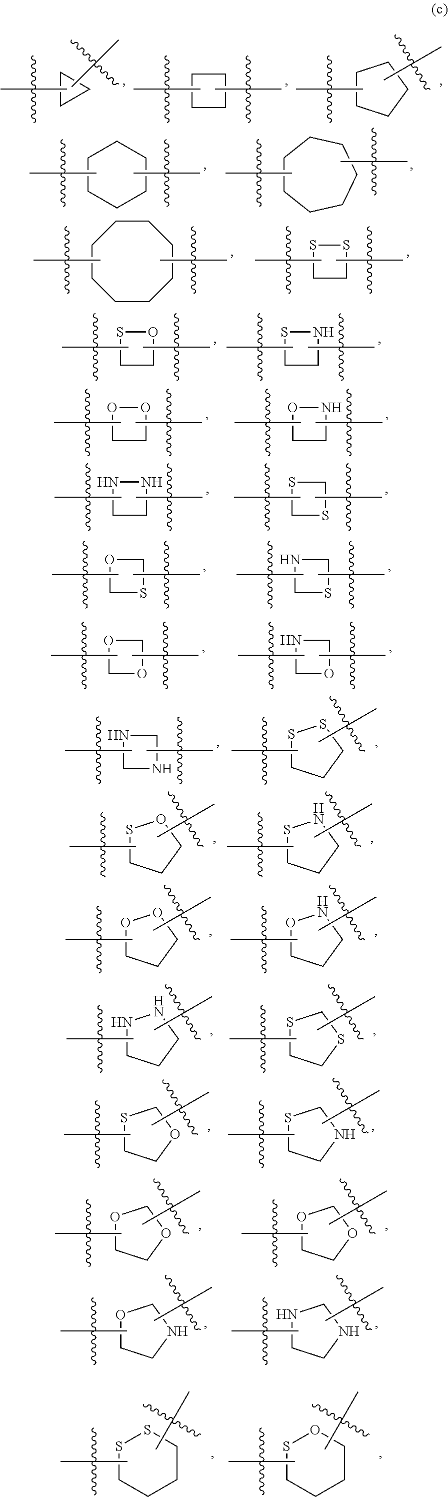

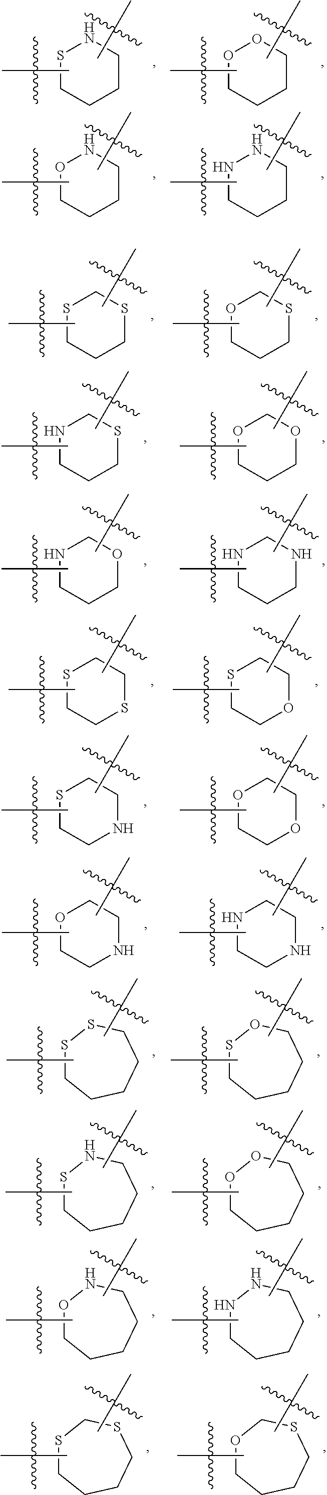

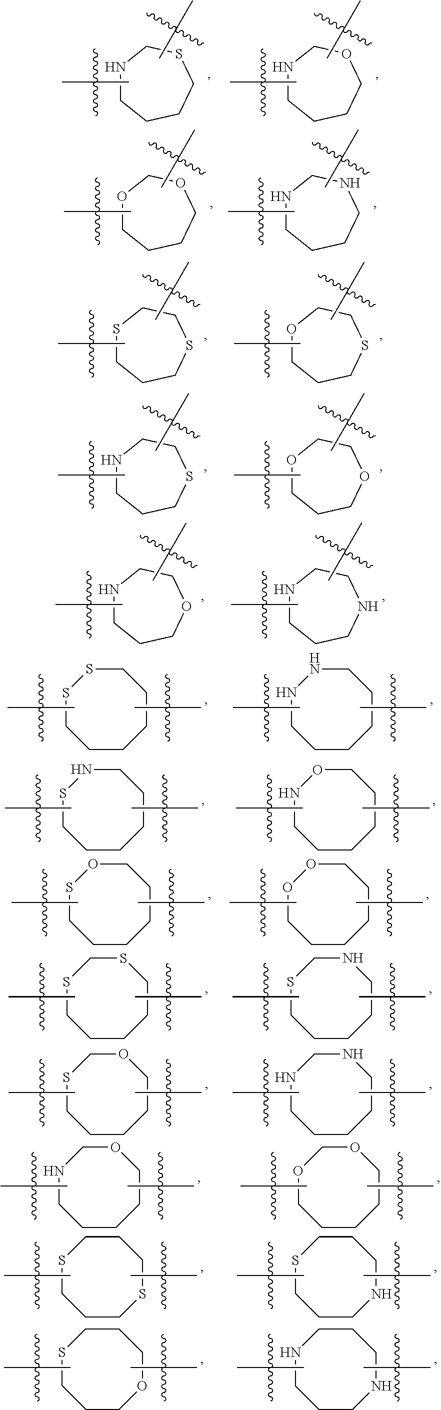

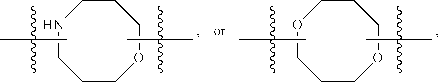

optionally substituted with halo or C1-C6 linear or branched alkyl.

In some variations of formula (33) described in the paragraphs above in which L is linker (l.1), ring A is substituted with halo. In some variations, the halo is F, Br, I, or Cl.

In some variations of formula (33) described in the paragraphs above in which L is linker (l.1), ring A is substituted with C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (33) described in the paragraphs above in which at least one of L1, L2, L3, and L4 is linker (l.1), n is 1-4, 1-3, 1-3, 1-2, 2-4, 2-3, 3-4, 1, 2, 3, or 4.

In some variations of formula (33) described in the paragraphs above, L1 is linker (l.1). In some variations of formula (33) described in the paragraphs above, L2 is linker (l.1). In some variations of formula (33) described in the paragraphs above, L3 is linker (l.1). In some variations of formula (33) described in the paragraphs above, L4 is linker (l.1). In some variations of formula (33) described in the paragraphs above, L1 and L2 are linker (l.1). In some variations of formula (33) described in the paragraphs above, L1 and L3 are linker (l.1). In some variations of formula (33) described in the paragraphs above, L1 and L4 are linker (l.1). In some variations of formula (33) described in the paragraphs above, L2 and L3 are linker (l.1). In some variations of formula (33) described in the paragraphs above, L2 and L4 are linker (l.1). In some variations of formula (33) described in the paragraphs above, L3 and L4 are linker (l.1). In some variations of formula (33) described in the paragraphs above, L1, L2, and L3 are linker (l.1). In some variations of formula (33) described in the paragraphs above, L1, L2, and L4 are linker (l.1). In some variations of formula (33) described in the paragraphs above, L1, L3, and L4 are linker (l.1). In some variations of formula (33) described in the paragraphs above, L2, L3, and L4 are linker (l.1). In some variations of formula (33) described in the paragraphs above, L1, L2, L3, and L4 are linker (l.1).

In some variations of formula (33) described in the paragraphs above, at least one of L1, L2, L3, and L4 is linker (l.2):

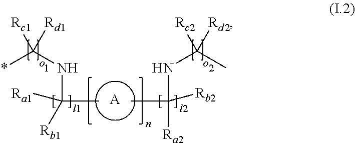

in which l₁, l₂, n, o₁, and o₂ independently are 1-4; ring A is aryl, heteroaryl, cycloalkyl, or heterocyclyl; for each independent instance of Rₐ₁ and R_{b1}, Rₐ₁ and R_{b1} (1) independently are H or CH₃, or (2) Rₐ₁ and R_{b1} independently are

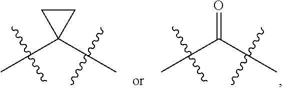

or (3) two of CRₐ₁R_{b1} are

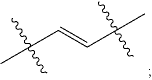 [SMILES: */C=C/*]

for each independent instance of Rₐ₂ and R_{b2}, Rₐ₂ and R_{b2} (1) independently are H or CH₃, or (2) Rₐ₂ and R_{b2} independently are

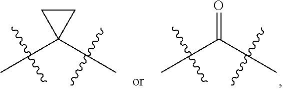

or (3) two of CRₐ₂R_{b2} are

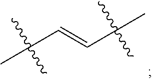 [SMILES: */C=C/*]

for each independent instance of R_{c1} and R_{d1}, R_{c1} and R_{d1} (1) independently are H or CH₃, or (2) R_{d} and Rₐ₁ independently are

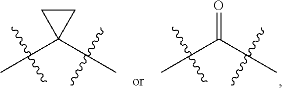

or (3) two of CR_{c1}R_{d1} are

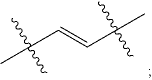 [SMILES: */C=C/*]

for each independent instance of R_{c2} and R_{d2}, R_{c2} and R_{d2} (1) independently are H or CH₃, or (2) R_{c2} and R_{d2} independently are

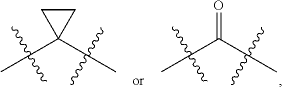

or (3) two of CR_{c2}R_{d2} are

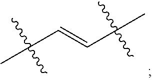 [SMILES: */C=C/*]

and * is the attachment site for the cationic dye moiety D.

In some variations of formula (33) in which at least one of L1, L2, L3, and L4 is linker (l.2), l₁ is 1-4, 1-3, 1-3, 1-2, 2-4, 2-3, 3-4, 1, 2, 3, or 4.

In some variations of formula (33) described in the paragraphs above in which at least one of L1, L2, L3, and L4 is linker (l.2), l₂ is 1-4, 1-3, 1-3, 1-2, 2-4, 2-3, 3-4, 1, 2, 3, or 4.

In some variations of formula (33) described in the paragraphs above in which at least one of L1, L2, L3, and L4 is linker (l.2), o₁ is 1-4, 1-3, 1-3, 1-2, 2-4, 2-3, 3-4, 1, 2, 3, or 4.

In some variations of formula (33) described in the paragraphs above in which at least one of L1, L2, L3, and L4 is linker (l.2), o₂ is 1-4, 1-3, 1-3, 1-2, 2-4, 2-3, 3-4, 1, 2, 3, or 4.

In some variations of formula (33) described in the paragraphs above in which at least one of L1, L2, L3, and L4 is linker (l.2), Rₐ₁ is H and R_{b1} is H. In some variations of formula (33) described in the paragraphs above in which at least one of L1, L2, L3, and L4 is linker (l.2), Rₐ₁ is H and R_{b1} is CH₃. In some variations of formula (33) described in the paragraphs above in which at least one of L1, L2, L3, and L4 is linker (l.2), Rₐ₁ and R_{b1} are

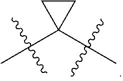 [SMILES: *C1(*)CC1]

In some variations of formula (33) described in the paragraphs above in which at least one of L1, L2, L3, and L4 is linker (l.2), Rₐ₁ and R_{b1} are

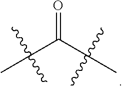 [SMILES: *C(*)=O]

In some variations of formula (33) described in the paragraphs above in which at least one of L1, L2, L3, and L4 is linker (l.2), two of CRₐ₁R_{b1} are

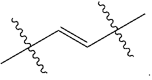 [SMILES: */C=C/*]

In some variations of formula (33) described in the paragraphs above in which at least one of L1, L2, L3, and L4 is linker (l.2), Rₐ₂ is H and R_{b2} is H. In some variations of formula (33) described in the paragraphs above in which at least one of L1, L2, L3, and L4 is linker (l.2), Rₐ₂ is H and R_{b2} is CH₃. In some variations of formula (33) described in the paragraphs above in which at least one of L1, L2, L3, and L4 is linker (l.2), Rₐ₂ and R_{b2} are

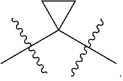 [SMILES: *C1(*)CC1]

In some variations of formula (33) described in the paragraphs above in which at least one of L1, L2, L3, and L4 is linker (l.2), Rₐₑ and R_{b2} are

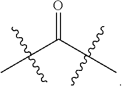 [SMILES: *C(*)=O]

In some variations of formula (33) described in the paragraphs above in which at least one of L1, L2, L3, and L4 is linker (l.2), two of CRₐ₂R_{b2} are

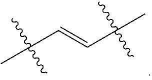 [SMILES: */C=C/*]

In some variations of formula (33) described in the paragraphs above in which at least one of L1, L2, L3, and L4 is linker (l.2), R_{c1} is H and R_{d1} is H. In some variations of formula (33) described in the paragraphs above in which at least one of L1, L2, L3, and L4 is linker (l.2), R_{c1} is H and R_{d1} is CH₃. In some variations of formula (33) described in the paragraphs above in which at least one of L1, L2, L3, and L4 is linker (l.2), R_{c1} and R_{d1} are

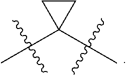 [SMILES: *C1(*)CC1]

In some variations of formula (33) described in the paragraphs above in which at least one of L1, L2, L3, and L4 is linker (l.2), R_{c1} and R_{d1} are

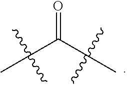 [SMILES: *C(*)=O]

In some variations of formula (33) described in the paragraphs above in which at least one of L1, L2, L3, and L4 is linker (l.2), two of CR_{c1}R_{d1} are

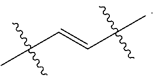 [SMILES: */C=C/*]

In some variations of formula (33) described in the paragraphs above in which at least one of L1, L2, L3, and L4 is linker (l.2), R_{c2} is H and R_{d2} is H. In some variations of formula (33) described in the paragraphs above in which at least one of L1, L2, L3, and L4 is linker (l.2), R_{c2} is H and R_{d2} is CH₃. In some variations of formula (33) described in the paragraphs above in which at least one of L1, L2, L3, and L4 is linker (l.2), R_{c2} and R_{d2} are

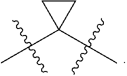 [SMILES: *C1(*)CC1]

In some variations of formula (33) described in the paragraphs above in which at least one of L1, L2, L3, and L4 is linker (l.2), R_{c2} and R_{d2} are

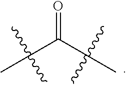 [SMILES: *C(*)=O]

In some variations of formula (33) described in the paragraphs above in which at least one of L1, L2, L3, and L4 is linker (l.2), two of CRₑ₂R_{d2} are

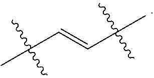 [SMILES: */C=C/*]

In some variations of formula (33) described in the paragraphs above in which at least one of L1, L2, L3, and L4 is linker (l.2), ring A is

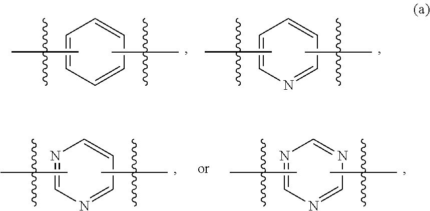

optionally substituted with halo or C1-C6 linear or branched alkyl;

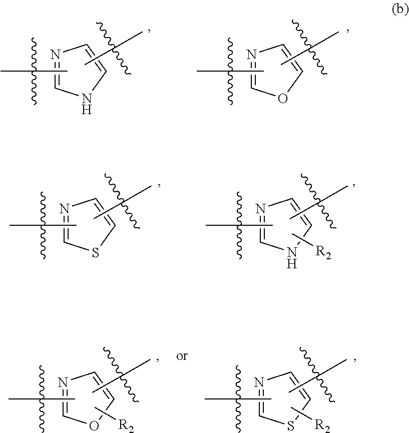

optionally substituted with halo or C1-C6 linear or branched alkyl; or

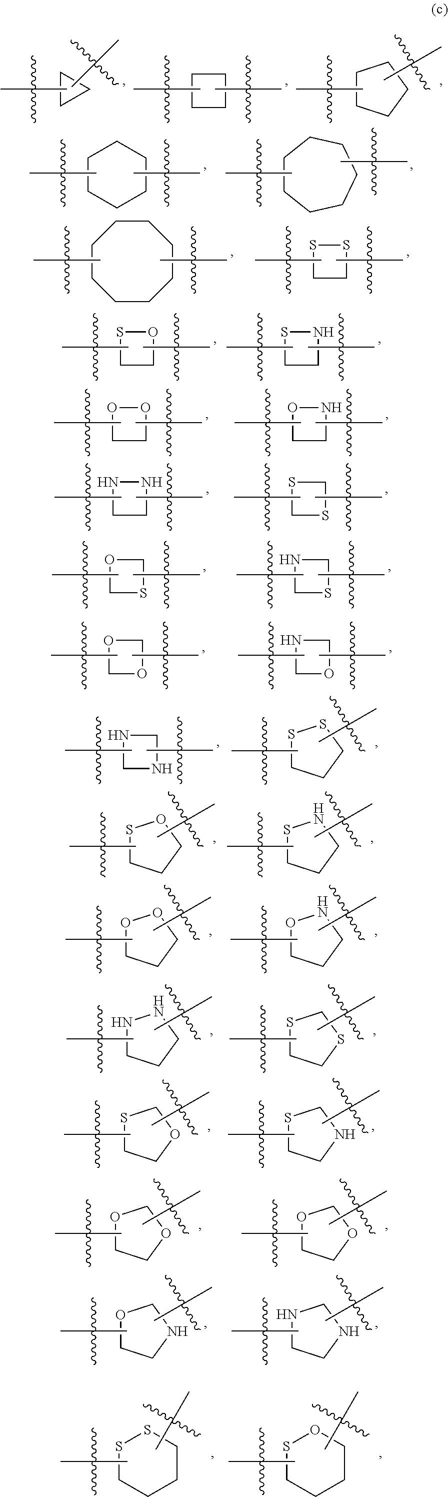

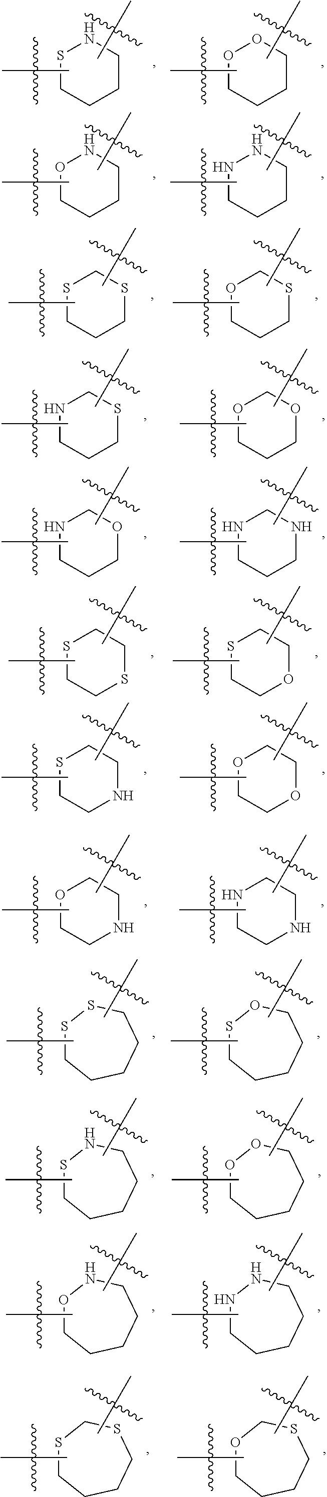

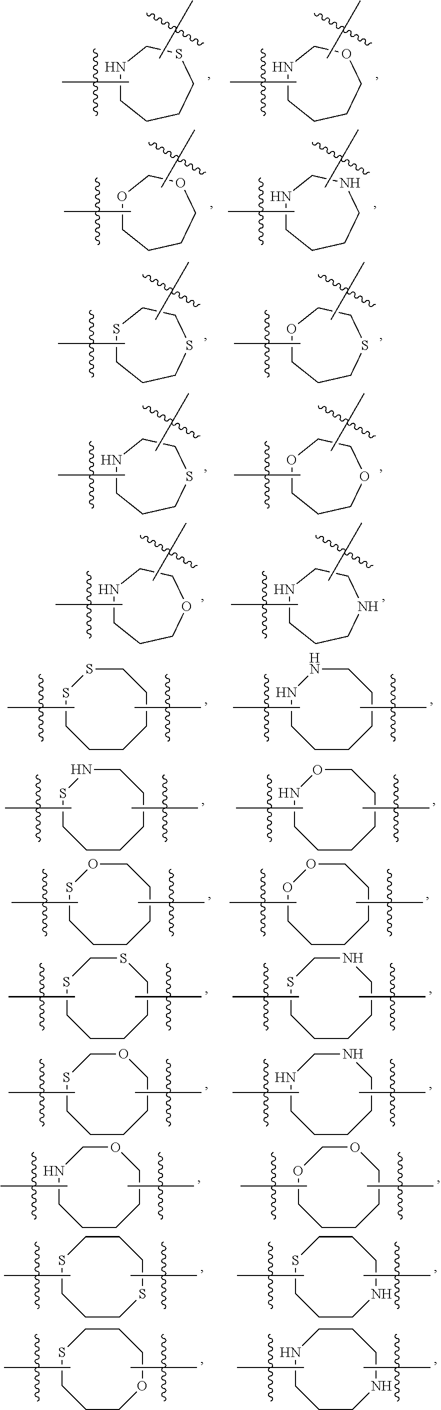

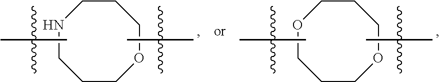

optionally substituted with halo or C1-C6 linear or branched alkyl.

In some variations of formula (33) described in the paragraphs above in which L is linker (l.2), ring A is substituted with halo. In some variations, the halo is F, Br, I, or Cl.

In some variations of formula (33) described in the paragraphs above in which L is linker (l.2), ring A is substituted with C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (33) described in the paragraphs above in which at least one of L1, L2, L3, and L4 is linker (l.2), n is 1-4, 1-3, 1-3, 1-2, 2-4, 2-3, 3-4, 1, 2, 3, or 4.

In some variations of formula (33) described in the paragraphs above, L1 is linker (l.2). In some variations of formula (33) described in the paragraphs above, L2 is linker (l.2). In some variations of formula (33) described in the paragraphs above, L3 is linker (l.2). In some variations of formula (33) described in the paragraphs above, L4 is linker (l.2). In some variations of formula (33) described in the paragraphs above, L1 and L2 are linker (l.2). In some variations of formula (33) described in the paragraphs above, L1 and L3 are linker (l.2). In some variations of formula (33) described in the paragraphs above, L1 and L4 are linker (l.2). In some variations of formula (33) described in the paragraphs above, L2 and L3 are linker (l.2). In some variations of formula (33) described in the paragraphs above, L2 and L4 are linker (l.2). In some variations of formula (33) described in the paragraphs above, L3 and L4 are linker (l.2). In some variations of formula (33) described in the paragraphs above, L1, L2, and L3 are linker (l.2). In some variations of formula (33) described in the paragraphs above, L1, L2, and L4 are linker (l.2). In some variations of formula (33) described in the paragraphs above, L1, L3, and L4 are linker (l.2). In some variations of formula (33) described in the paragraphs above, L2, L3, and L4 are linker (l.2). In some variations of formula (33) described in the paragraphs above, L1, L2, L3, and L4 are linker (l.2).

In some variations of formula (33) described in the paragraphs above, at least one of L1, L2, L3, and L4 is linker (m.1):

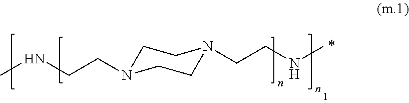

in which n is 0-6, n₁ is 1-4, and * is the attachment site for the cationic dye moiety D.

In some variations of formula (33) in which at least one of L1, L2, L3, and L4 is linker (m.1), n is 0-6, 0-5, 0-4, 0-3, 0-2, 0-1, 1-6, 1-5, 1-4, 1-3, 1-2, 2-6, 2-5, 2-4, 2-3, 3-6, 3-5, 3-4, 4-6, 4-5, 5-6, 0, 1, 2, 3, 4, 5, or 6.

In some variations of formula (33) described in the paragraphs above in which at least one of L1, L2, L3, and L4 is linker (m.1), n₁ is 1-4, 1-3, 1-2, 2-4, 2-3, 3-4, 1, 2, 3, or 4.

In some variations of formula (33) described in the paragraphs above, L1 is linker (m.1). In some variations of formula (33) described in the paragraphs above, L2 is linker (m.1). In some variations of formula (33) described in the paragraphs above, L3 is linker (m.1). In some variations of formula (33) described in the paragraphs above, L4 is linker (m.1). In some variations of formula (33) described in the paragraphs above, L1 and L2 are linker (m.1). In some variations of formula (33) described in the paragraphs above, L1 and L3 are linker (m.1). In some variations of formula (33) described in the paragraphs above, L1 and L4 are linker (m.1). In some variations of formula (33) described in the paragraphs above, L2 and L3 are linker (m.1). In some variations of formula (33) described in the paragraphs above, L2 and L4 are linker (m.1). In some variations of formula (33) described in the paragraphs above, L3 and L4 are linker (m.1). In some variations of formula (33) described in the paragraphs above, L1, L2, and L3 are linker (m.1). In some variations of formula (33) described in the paragraphs above, L1, L2, and L4 are linker (m.1). In some variations of formula (33) described in the paragraphs above, L1, L3, and L4 are linker (m.1). In some variations of formula (33) described in the paragraphs above, L2, L3, and L4 are linker (m.1). In some variations of formula (33) described in the paragraphs above, L1, L2, L3, and L4 are linker (m.1).

In some variations of formula (33) described in the paragraphs above, at least one of L1, L2, L3, and L4 is linker (n.1):

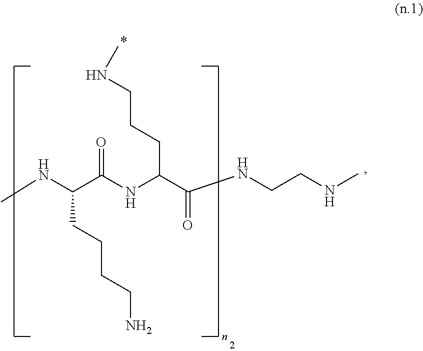

in which n₂ is 1-5 and * is the attachment site for the cationic dye moiety D.

In some variations of formula (33) in which at least one of L1, L2, L3, and L4 is linker (n.1), n₂ is 1-5, 1-4, 1-3, 1-2, 2-5, 2-4, 2-3, 3-5, 3-4, 4-5, 1, 2, 3, 4, or 5.

In some variations of formula (33) described in the paragraphs above, L1 is linker (n.1). In some variations of formula (33) described in the paragraphs above, L2 is linker (n.1). In some variations of formula (33) described in the paragraphs above, L3 is linker (n.1). In some variations of formula (33) described in the paragraphs above, L4 is linker (n.1). In some variations of formula (33) described in the paragraphs above, L1 and L2 are linker (n.1). In some variations of formula (33) described in the paragraphs above, L1 and L3 are linker (n.1). In some variations of formula (33) described in the paragraphs above, L1 and L4 are linker (n.1). In some variations of formula (33) described in the paragraphs above, L2 and L3 are linker (n.1). In some variations of formula (33) described in the paragraphs above, L2 and L4 are linker (n.1). In some variations of formula (33) described in the paragraphs above, L3 and L4 are linker (n.1). In some variations of formula (33) described in the paragraphs above, L1, L2, and L3 are linker (n.1). In some variations of formula (33) described in the paragraphs above, L1, L2, and L4 are linker (n.1). In some variations of formula (33) described in the paragraphs above, L1, L3, and L4 are linker (n.1). In some variations of formula (33) described in the paragraphs above, L2, L3, and L4 are linker (n.1). In some variations of formula (33) described in the paragraphs above, L1, L2, L3, and L4 are linker (n.1).

In some variations of formula (33) described in the paragraphs above, at least one of L1, L2, L3, and L4 is linker (n.2):

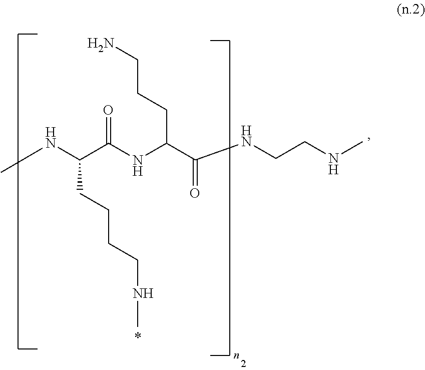

in which n₂ is 1-5 and * is the attachment site for the cationic dye moiety D.

In some variations of formula (33) in which at least one of L1, L2, L3, and L4 is linker (n.2), n₂ is 1-5, 1-4, 1-3, 1-2, 2-5, 2-4, 2-3, 3-5, 3-4, 4-5, 1, 2, 3, 4, or 5.

In some variations of formula (33) described in the paragraphs above, L1 is linker (n.2). In some variations of formula (33) described in the paragraphs above, L2 is linker (n.2). In some variations of formula (33) described in the paragraphs above, L3 is linker (n.2). In some variations of formula (33) described in the paragraphs above, L4 is linker (n.2). In some variations of formula (33) described in the paragraphs above, L1 and L2 are linker (n.2). In some variations of formula (33) described in the paragraphs above, L1 and L3 are linker (n.2). In some variations of formula (33) described in the paragraphs above, L1 and L4 are linker (n.2). In some variations of formula (33) described in the paragraphs above, L2 and L3 are linker (n.2). In some variations of formula (33) described in the paragraphs above, L2 and L4 are linker (n.2). In some variations of formula (33) described in the paragraphs above, L3 and L4 are linker (n.2). In some variations of formula (33) described in the paragraphs above, L1, L2, and L3 are linker (n.2). In some variations of formula (33) described in the paragraphs above, L1, L2, and L4 are linker (n.2). In some variations of formula (33) described in the paragraphs above, L1, L3, and L4 are linker (n.2). In some variations of formula (33) described in the paragraphs above, L2, L3, and L4 are linker (n.2). In some variations of formula (33) described in the paragraphs above, L1, L2, L3, and L4 are linker (n.2).

In some variations of formula (33) described in the paragraphs above, at least one of L1, L2, L3, and L4 is linker (o):

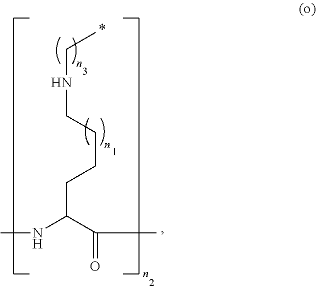

in which in which n₁ is 0-5, n₂ is 1-5, n3 is 0-5, and * is the attachment site for the cationic dye moiety D.

In some variations of formula (33) in which at least one of L1, L2, L3, and L4 is linker (o), n₁ is 0-5, 0-4, 0-3, 0-2, 0-1, 1-5, 1-4, 1-3, 1-2, 2-5, 2-4, 2-3, 3-5, 3-4, 4-5, 0, 1, 2, 3, 4, or 5.

In some variations of formula (33) described in the paragraphs above in which at least one of L1, L2, L3, and L4 is linker (o), n₂ is 1-5, 1-4, 1-3, 1-2, 2-5, 2-4, 2-3, 3-5, 3-4, 4-5, 1, 2, 3, 4, or 5.

In some variations of formula (33) described in the paragraphs above in which at least one of L1, L2, L3, and L4 is linker (o), n₃ is 0-5, 0-4, 0-3, 0-2, 0-1, 1-5, 1-4, 1-3, 1-2, 2-5, 2-4, 2-3, 3-5, 3-4, 4-5, 0, 1, 2, 3, 4, or 5.

In some variations of formula (33) described in the paragraphs above, L1 is linker (o). In some variations of formula (33) described in the paragraphs above, L2 is linker (o). In some variations of formula (33) described in the paragraphs above, L3 is linker (o). In some variations of formula (33) described in the paragraphs above, L4 is linker (o). In some variations of formula (33) described in the paragraphs above, L1 and L2 are linker (o). In some variations of formula (33) described in the paragraphs above, L1 and L3 are linker (o). In some variations of formula (33) described in the paragraphs above, L1 and L4 are linker (o). In some variations of formula (33) described in the paragraphs above, L2 and L3 are linker (o). In some variations of formula (33) described in the paragraphs above, L2 and L4 are linker (o). In some variations of formula (33) described in the paragraphs above, L3 and L4 are linker (o). In some variations of formula (33) described in the paragraphs above, L1, L2, and L3 are linker (o). In some variations of formula (33) described in the paragraphs above, L1, L2, and L4 are linker (o). In some variations of formula (33) described in the paragraphs above, L1, L3, and L4 are linker (o). In some variations of formula (33) described in the paragraphs above, L2, L3, and L4 are linker (o). In some variations of formula (33) described in the paragraphs above, L1, L2, L3, and L4 are linker (o).

In some variations of formula (33) described in the paragraphs above, at least one of L1, L2, L3, and L4 is linker (p):

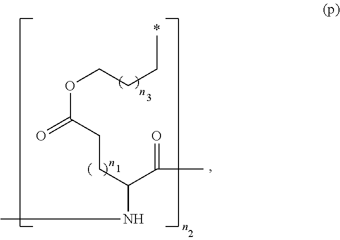

in which n₁ is 0-5, n₂ is 1-5, n3 is 0-5, and * is the attachment site for the cationic dye moiety D.

In some variations of formula (33) described above in which at least one of L1, L2, L3, and L4 is linker (p), n₁ is 0-5, 0-4, 0-3, 0-2, 0-1, 1-5, 1-4, 1-3, 1-2, 2-5, 2-4, 2-3, 3-5, 3-4, 4-5, 0, 1, 2, 3, 4, or 5.

In some variations of formula (33) described above in which at least one of L1, L2, L3, and L4 is linker (p), n₂ is 1-5, 1-4, 1-3, 1-2, 2-5, 2-4, 2-3, 3-5, 3-4, 4-5, 1, 2, 3, 4, or 5.

In some variations of formula (33) described above in which at least one of L1, L2, L3, and L4 is linker (p), n₃ is 0-5, 0-4, 0-3, 0-2, 0-1, 1-5, 1-4, 1-3, 1-2, 2-5, 2-4, 2-3, 3-5, 3-4, 4-5, 0, 1, 2, 3, 4, or 5.

In some variations of formula (33) described in the paragraphs above, L1 is linker (p). In some variations of formula (33) described in the paragraphs above, L2 is linker (p). In some variations of formula (33) described in the paragraphs above, L3 is linker (p). In some variations of formula (33) described in the paragraphs above, L4 is linker (p). In some variations of formula (33) described in the paragraphs above, L1 and L2 are linker (p). In some variations of formula (33) described in the paragraphs above, L1 and L3 are linker (p). In some variations of formula (33) described in the paragraphs above, L1 and L4 are linker (p). In some variations of formula (33) described in the paragraphs above, L2 and L3 are linker (p). In some variations of formula (33) described in the paragraphs above, L2 and L4 are linker (p). In some variations of formula (33) described in the paragraphs above, L3 and L4 are linker (p). In some variations of formula (33) described in the paragraphs above, L1, L2, and L3 are linker (p). In some variations of formula (33) described in the paragraphs above, L1, L2, and L4 are linker (p). In some variations of formula (33) described in the paragraphs above, L1, L3, and L4 are linker (p). In some variations of formula (33) described in the paragraphs above, L2, L3, and L4 are linker (p). In some variations of formula (33) described in the paragraphs above, L1, L2, L3, and L4 are linker (p).

In some variations of formula (33) described in the paragraphs above, at least one of L1, L2, L3, and L4 is linker (q):

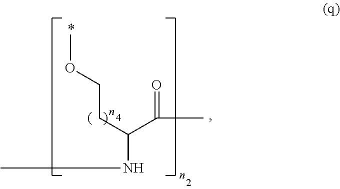

in which n₄ is 0-5, n₂ is 1-5, and * is the attachment site for the cationic dye moiety D.

In some variations of formula (33) in which at least one of L1, L2, L3, and L4 is linker (o), n₄ is 0-5, 0-4, 0-3, 0-2, 0-1, 1-5, 1-4, 1-3, 1-2, 2-5, 2-4, 2-3, 3-5, 3-4, 4-5, 0, 1, 2, 3, 4, or 5.

In some variations of formula (33) described above in which at least one of L1, L2, L3, and L4 is linker (q), n₂ is 1-5, 1-4, 1-3, 1-2, 2-5, 2-4, 2-3, 3-5, 3-4, 4-5, 1, 2, 3, 4, or 5.

In some variations of formula (33) described in the paragraphs above, L1 is linker (q). In some variations of formula (33) described in the paragraphs above, L2 is linker (q). In some variations of formula (33) described in the paragraphs above, L3 is linker (q). In some variations of formula (33) described in the paragraphs above, L4 is linker (q). In some variations of formula (33) described in the paragraphs above, L1 and L2 are linker (q). In some variations of formula (33) described in the paragraphs above, L1 and L3 are linker (q). In some variations of formula (33) described in the paragraphs above, L1 and L4 are linker (q). In some variations of formula (33) described in the paragraphs above, L2 and L3 are linker (q). In some variations of formula (33) described in the paragraphs above, L2 and L4 are linker (q). In some variations of formula (33) described in the paragraphs above, L3 and L4 are linker (q). In some variations of formula (33) described in the paragraphs above, L1, L2, and L3 are linker (q). In some variations of formula (33) described in the paragraphs above, L1, L2, and L4 are linker (q). In some variations of formula (33) described in the paragraphs above, L1, L3, and L4 are linker (q). In some variations of formula (33) described in the paragraphs above, L2, L3, and L4 are linker (q). In some variations of formula (33) described in the paragraphs above, L1, L2, L3, and L4 are linker (q).

In some variations of formula (33) described in the paragraphs above, at least one of L1, L2, L3, and L4 is linker (r):

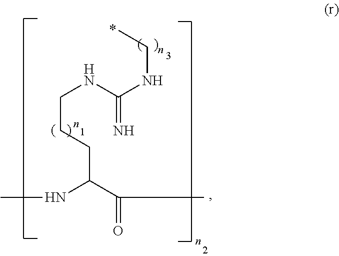

in which n₁ is 0-5, n₂ is 1-5, n3 is 0-5, and * is the attachment site for the cationic dye moiety D.

In some of variations of formula (33) in which at least one of L1, L2, L3, and L4 is linker (r), n₁ is 0-5, 0-4, 0-3, 0-2, 0-1, 1-5, 1-4, 1-3, 1-2, 2-5, 2-4, 2-3, 3-5, 3-4, 4-5, 0, 1, 2, 3, 4, or 5.

In some of variations of formula (33) described in the paragraphs above in which at least one of L1, L2, L3, and L4 is linker (r), n₂ is 1-5, 1-4, 1-3, 1-2, 2-5, 2-4, 2-3, 3-5, 3-4, 4-5, 1, 2, 3, 4, or 5.

In some of variations of formula (33) in which at least one of L1, L2, L3, and L4 is linker (r), n3 is 0-5, 0-4, 0-3, 0-2, 0-1, 1-5, 1-4, 1-3, 1-2, 2-5, 2-4, 2-3, 3-5, 3-4, 4-5, 0, 1, 2, 3, 4, or 5.

In some variations of formula (33) described in the paragraphs above, L1 is linker (r). In some variations of formula (33) described in the paragraphs above, L2 is linker (r). In some variations of formula (33) described in the paragraphs above, L3 is linker (r). In some variations of formula (33) described in the paragraphs above, L4 is linker (r). In some variations of formula (33) described in the paragraphs above, L1 and L2 are linker (r). In some variations of formula (33) described in the paragraphs above, L1 and L3 are linker (r). In some variations of formula (33) described in the paragraphs above, L1 and L4 are linker (r). In some variations of formula (33) described in the paragraphs above, L2 and L3 are linker (r). In some variations of formula (33) described in the paragraphs above, L2 and L4 are linker (r). In some variations of formula (33) described in the paragraphs above, L3 and L4 are linker (r). In some variations of formula (33) described in the paragraphs above, L1, L2, and L3 are linker (r). In some variations of formula (33) described in the paragraphs above, L1, L2, and L4 are linker (r). In some variations of formula (33) described in the paragraphs above, L1, L3, and L4 are linker (r). In some variations of formula (33) described in the paragraphs above, L2, L3, and L4 are linker (r). In some variations of formula (33) described in the paragraphs above, L1, L2, L3, and L4 are linker (r).

In some variations of formula (33) described in the paragraphs above, at least one of L1, L2, L3, and L4 is linker (s):

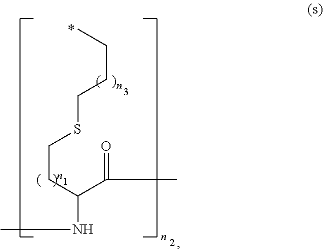

in which n₁ is 0-5, n₂ is 1-5, n₃ is 0-5, and * is the attachment site for the cationic dye moiety D.

In some variations of formula (33) described in the paragraphs above, L1 is linker (s). In some variations of formula (33) described in the paragraphs above, L2 is linker (s). In some variations of formula (33) described in the paragraphs above, L3 is linker (s). In some variations of formula (33) described in the paragraphs above, L4 is linker (s). In some variations of formula (33) described in the paragraphs above, L1 and L2 are linker (s). In some variations of formula (33) described in the paragraphs above, L1 and L3 are linker (s). In some variations of formula (33) described in the paragraphs above, L1 and L4 are linker (s). In some variations of formula (33) described in the paragraphs above, L2 and L3 are linker (s). In some variations of formula (33) described in the paragraphs above, L2 and L4 are linker (s). In some variations of formula (33) described in the paragraphs above, L3 and L4 are linker (s). In some variations of formula (33) described in the paragraphs above, L1, L2, and L3 are linker (s). In some variations of formula (33) described in the paragraphs above, L1, L2, and L4 are linker (s). In some variations of formula (33) described in the paragraphs above, L1, L3, and L4 are linker (s). In some variations of formula (33) described in the paragraphs above, L2, L3, and L4 are linker (s). In some variations of formula (33) described in the paragraphs above, L1, L2, L3, and L4 are linker (s).

In some variations of formula (33) described in the paragraphs above, L1 is absent. In some variations of formula (33) described in the paragraphs above, L2 is absent. In some variations of formula (33) described in the paragraphs above, L3 is absent. In some variations of formula (33) described in the paragraphs above, L4 is absent. In some variations of formula (33) described in the paragraphs above, L1 and L2 are absent. In some variations of formula (33) described in the paragraphs above, L1 and L3 are absent. In some variations of formula (33) described in the paragraphs above, L1 and L4 are absent. In some variations of formula (33) described in the paragraphs above, L2 and L3 are absent. In some variations of formula (33) described in the paragraphs above, L2 and L4 are absent. In some variations of formula (33) described in the paragraphs above, L3 and L4 are absent. In some variations of formula (33) described in the paragraphs above, L1, L2, and L3 are absent. In some variations of formula (33) described in the paragraphs above, L1, L2, and L4 are absent. In some variations of formula (33) described in the paragraphs above, L1, L3, and L4 are absent. In some variations of formula (33) described in the paragraphs above, L2, L3, and L4 are absent. In some variations of formula (33) described in the paragraphs above, L1, L2, L3, and L4 are absent.

In some variations of formula (33), each of L1, L2, L3, and L4 is a different linker. In some variations of formula (33), each of L1, L2, L3, and L4 is the same linker. In some variations of formula (33), L1 is a first linker and each of L2, L3, and L4 is the same second linker, wherein the first and second linkers are different. In some variations of formula (33), L2 is a first linker, and each of L1, L3, and L4 is a second linker, wherein the first and second linkers are different. In some variations of formula (33), L3 is a first linker, and each of L1, L2, and L4 is a second linker, wherein the first and second linkers are different. In some variations of formula (33), L4 is a first linker, and each of L1, L2, and L3 is a second linker, wherein the first and second linkers are different. In some variations of formula (33), each of L1 and L2 is the same first linker, and each of L3 and L4 is the same second linker, wherein the first and second linkers are different. In some variations of formula (33), each of L1 and L2 is the same first linker, L3 is a second linker, and L4 is a third linker, wherein the first, second, and third linkers are different. In some variations of formula (33), each of L1 and L3 is the same first linker, and each of L2 and L4 is the same second linker, wherein the first and second linkers are different. In some variations of formula (33), each of L1 and L3 is the same first linker, L2 is a second linker, and L4 is a third linker, wherein the first, second, and third linkers are different. In some variations of formula (33), each of L1 and L4 is the same first linker, and each of L2 and L3 is the same second linker, wherein the first and second linkers are different. In some variations of formula (33), each of L1 and L4 is the same first linker, L2 is a different second linker, and L3 is a different third linker, wherein the first, second, and third linkers are different. In some variations of formula (33), each of L2 and L3 is the same first linker, and each of L1 and L4 is the same second linker, wherein the first and second linkers are different.

In some variations of formula (33) described in the paragraphs above, D1 is safranin-O. In some variations of formula (33) described in the paragraphs above, D2 is safranin-O. In some variations of formula (33) described in the paragraphs above, D3 is safranin-O. In some variations of formula (33) described in the paragraphs above, D4 is safranin-O. In some variations of formula (33) described in the paragraphs above, D1 and D2 are safranin-O. In some variations of formula (33) described in the paragraphs above, D1 and D3 are safranin-O. In some variations of formula (33) described in the paragraphs above, D1 and D4 are safranin-O. In some variations of formula (33) described in the paragraphs above, D2 and D3 are safranin-O. In some variations of formula (33) described in the paragraphs above, D2 and D4 are safranin-O. In some variations of formula (33) described in the paragraphs above, D3 and D4 are safranin-O. In some variations of formula (33) described in the paragraphs above, D1, D2, and D3 are safranin-O. In some variations of formula (33) described in the paragraphs above, D1, D2, and D4 are safranin-O. In some variations of formula (33) described in the paragraphs above, D1, D3, and D4 are safranin-O. In some variations of formula (33) described in the paragraphs above, D2, D3, and D4 are safranin-O. In some variations of formula (33) described in the paragraphs above, D1, D2, D3, and D4 are safranin-O.

In some variations of formula (33) described in the paragraph above, the pendant phenyl ring of D1 is unsubstituted. In some variations of formula (33) described in the paragraph above, the pendant phenyl ring of D1 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D1 is substituted, the substituents are selected independently from NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D1 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (33) described in the two paragraphs above, the pendant phenyl ring of D2 is unsubstituted. In some variations of formula (33) described in the paragraph above, the pendant phenyl ring of D2 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D2 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D2 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (33) described in the three paragraphs above, the pendant phenyl ring of D3 is unsubstituted. In some variations of formula (33) described in the paragraph above, the pendant phenyl ring of D3 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D3 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D3 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (33) described in the three paragraphs above, the pendant phenyl ring of D4 is unsubstituted. In some variations of formula (33) described in the paragraph above, the pendant phenyl ring of D4 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D4 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D4 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations, cationic dye multimers are cyclic, as illustrated by formula (34):

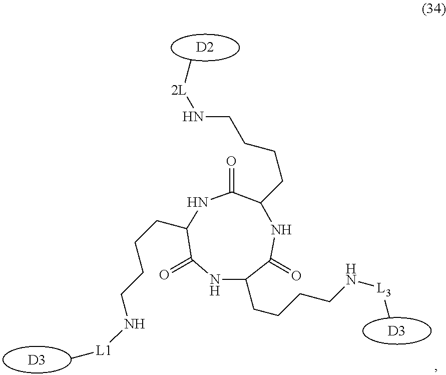

wherein at least one of D1, D2, and D3 is a cationic dye moiety and one or more of L1, L2, and L3 are absent or each of L1, L2, and L3 is a linker independently selected from linker (a.1), linker (a.2), linker (b.1), linker (c.1), linker (c.2), linker (d), linker (e.1), linker (f.1), linker (f.2), linker (g.1), linker (g.2), linker (h.1), linker (h.2), linker (i.1), linker (i.2), linker (j.1), linker (j.2), linker (k), linker (l.1), linker (l.2), is linker (m.1), linker (n.1), linker (n.2), linker (o), linker (p), linker (q), linker (r), and linker (s), described above.

In some variations of formula (34) described in the paragraphs above, at least one of D1, D2, and D3 is a different cationic dye moiety. In some variations of formula (34) described in the paragraphs above, at least one of D1, D2, and D3 is the same cationic dye moiety. In some variations of formula (34) described in the paragraphs above, at least one of D1 and D2 is the same first cationic dye moiety and D3 is a second cationic dye moiety, wherein the first and second cationic dye moieties are different. In some variations of formula (34) described in the paragraphs above, at least one of D1 and D3 is the same first cationic dye moiety and D2 is a second cationic dye moiety, wherein the first and second cationic dye moieties are different. In some variations of formula (34) described in the paragraphs above, at least one of D2 and D3 is the same first cationic dye moiety and D1 is a second cationic dye moiety, wherein the first and second cationic dye moieties are different.

In some variations of formula (34) described in the paragraphs above, at least one of D1, D2, and D3 is independently are selected from the group consisting of safranin-O, toluidine blue, azure A, azure B, azure C, acridine orange, acriflavine, and methylene blue.

In some variations of formula (34) described in the paragraphs above, at least one of L1, L2, and L3 is linker (a.1):

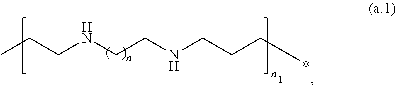

in which n is 1-6, n₁ is 1-4, and * is the attachment site for the cationic dye moiety D.

In some variations of formula (34) in which at least one of L1, L2, and L3 is linker (a.1), n is 1-6, 1-5, 1-4, 1-3, 1-2, 2-6, 2-5, 2-4, 2-3, 3-6, 3-5, 3-4, 4-6, 4-5, 5-6, 1, 2, 3, 4, 5, or 6.

In some variations of formula (34) described in the paragraphs above in which at least one of L1, L2, and L3 is linker (a.1), n₁ is 1-4, 1-3, 1-2, 2-4, 2-3, 3-4, 1, 2, 3, or 4.

In some variations of formula (34) described in the paragraphs above, L1 is linker (a.1). In some variations of formula (34) described in the paragraphs above, L2 is linker (a.1). In some variations of formula (34) described in the paragraphs above, L3 is linker (a.1). In some variations of formula (34) described in the paragraphs above, L1 and L2 are linker (a.1). In some variations of formula (34) described in the paragraphs above, L1 and L3 are linker (a.1). In some variations of formula (34) described in the paragraphs above, L2 and L3 are linker (a.1). In some variations of formula (34) described in the paragraphs above, L1, L2, and L3 are linker (a.1).

In some variations of formula (34) described in the paragraphs above, at least one of L1, L2, and L3 is linker (a.2):

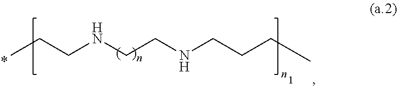

in which n is 1-6, n₁ is 1-4, and * is the attachment site for the cationic dye moiety D.

In some variations of formula (34) in which at least one of L1, L2, and L3 is linker (a.2), n is 1-6, 1-5, 1-4, 1-3, 1-2, 2-6, 2-5, 2-4, 2-3, 3-6, 3-5, 3-4, 4-6, 4-5, 5-6, 1, 2, 3, 4, 5, or 6.

In some variations of formula (34) described in the paragraphs above in which at least one of L1, L2, and L3 is linker (a.2), n₁ is 1-4, 1-3, 1-2, 2-4, 2-3, 3-4, 1, 2, 3, or 4.

In some variations of formula (34) described in the paragraphs above, L1 is linker (a.2). In some variations of formula (34) described in the paragraphs above, L2 is linker (a.2). In some variations of formula (34) described in the paragraphs above, L3 is linker (a.2). In some variations of formula (34) described in the paragraphs above, L1 and L2 are linker (a.2). In some variations of formula (34) described in the paragraphs above, L1 and L3 are linker (a.2). In some variations of formula (34) described in the paragraphs above, L2 and L3 are linker (a.2). In some variations of formula (34) described in the paragraphs above, L1, L2, and L3 are linker (a.2).

In some variations of formula (34) described in the paragraphs above, at least one of L1, L2, and L3 is linker (b.1):

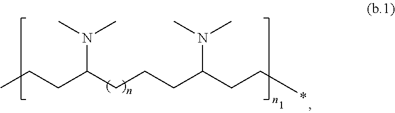

in which n is 0-6, n₁ is 1-4, and * is the attachment site for the cationic dye moiety D.

In some variations of formula (34) in which at least one of L1, L2, and L3 is linker (b.1), n is 0-6, 0-5, 0-4, 0-3, 0-2, 0-1, 1-6, 1-5, 1-4, 1-3, 1-2, 2-6, 2-5, 2-4, 2-3, 3-6, 3-5, 3-4, 4-6, 4-5, 5-6, 0, 1, 2, 3, 4, 5, or 6.

In some variations of formula (34) described in the paragraphs above in which at least one of L1, L2, and L3 is linker (b.1), n₁ is 1-4, 1-3, 1-2, 2-4, 2-3, 3-4, 1, 2, 3, or 4.

In some variations of formula (34) described in the paragraphs above, at least one of L1, L2, and L3 is linker (c.1):

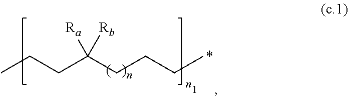

in which n is 0-6, n₁ is 1-4, either (1) Rₐ and R_{b} independently are H or CH₃ or (2) Rₐ and R_{b} are

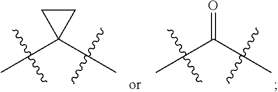

or (3) two of CRₐR_{b} are

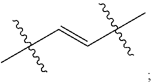 [SMILES: */C=C/*]

and * is the attachment site for the cationic dye moiety D.

In some variations of formula (34) in which at least one of L1, L2, and L3 is linker (c.1), n is 0-6, 0-5, 0-4, 0-3, 0-2, 0-1, 1-6, 1-5, 1-4, 1-3, 1-2, 2-6, 2-5, 2-4, 2-3, 3-6, 3-5, 3-4, 4-6, 4-5, 5-6, 0, 1, 2, 3, 4, 5, or 6.

In some variations of formula (34) described in the paragraphs above in which at least one of L1, L2, and L3 is linker (c.1), n₁ is 1-4, 1-3, 1-2, 2-4, 2-3, 3-4, 1, 2, 3, or 4.

In some variations of formula (34) described in the paragraphs above in which at least one of L1, L2, and L3 is linker (c.1), Rₐ is H and R_{b} is H. In some variations of formula (34) described in the paragraphs above in which at least one of L1, L2, and L3 is linker (c.1), Rₐ is H and R_{b} is CH₃. In some variations of formula (34) described in the paragraphs above in which at least one of L1, L2, and L3 is linker (c.1), Rₐ and R_{b} are

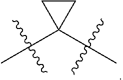 [SMILES: *C1(*)CC1]

In some variations of formula (34) described in the paragraphs above in which at least one of L1, L2, and L3 is linker (c.1), Rₐ and R_{b} are

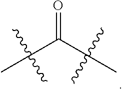 [SMILES: *C(*)=O]

In some variations of formula (34) described in the paragraphs above in which at least one of L1, L2, and L3 is linker (c.1), two of CRₐR_{b} are

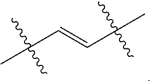 [SMILES: */C=C/*]

In some variations of formula (34) described in the paragraphs above, L1 is linker (c.1). In some variations of formula (34) described in the paragraphs above, L2 is linker (c.1). In some variations of formula (34) described in the paragraphs above, L3 is linker (c.1). In some variations of formula (34) described in the paragraphs above, L1 and L2 are linker (c.1). In some variations of formula (34) described in the paragraphs above, L1 and L3 are linker (c.1). In some variations of formula (34) described in the paragraphs above, L2 and L3 are linker (c.1). In some variations of formula (34) described in the paragraphs above, L1, L2, and L3 are linker (c.1).

In some variations of formula (34) described in the paragraphs above in which at least one of L1, L2, and L3 is linker (c.1), L is

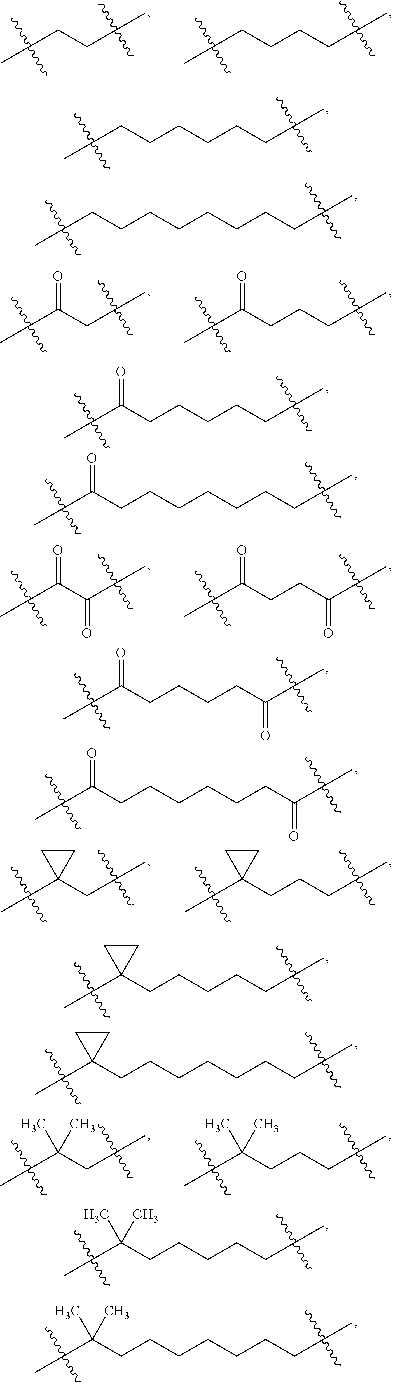

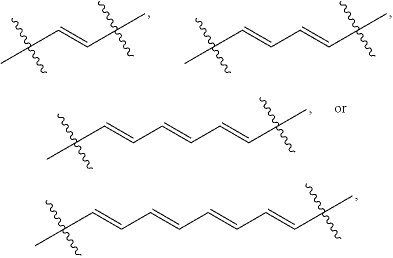

In some variations of formula (34) described in the paragraphs above, L1 is linker (c.1). In some variations of formula (34) described in the paragraphs above, L2 is linker (c.1). In some variations of formula (34) described in the paragraphs above, L3 is linker (c.1). In some variations of formula (34) described in the paragraphs above, L1 and L2 are linker (c.1). In some variations of formula (34) described in the paragraphs above, L1 and L3 are linker (c.1). In some variations of formula (34) described in the paragraphs above, L2 and L3 are linker (c.1). In some variations of formula (34) described in the paragraphs above, L1, L2, and L3 are linker (c.1).

In some variations of formula (34) described in the paragraphs above, at least one of L1, L2, and L3 is linker (c.2):

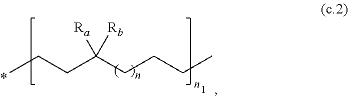

in which n is 0-6, n₁ is 1-4, either (1) Rₐ and R_{b} independently are H or CH₃ or (2) Rₐ and R_{b} are

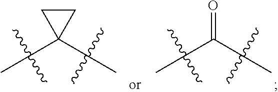

or (3) two of CRₐR_{b} are

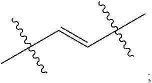 [SMILES: */C=C/*]

and * is the attachment site for the cationic dye moiety D.

In some variations of formula (34) in which at least one of L1, L2, and L3 is linker (c.2), n is 0-6, 0-5, 0-4, 0-3, 0-2, 0-1, 1-6, 1-5, 1-4, 1-3, 1-2, 2-6, 2-5, 2-4, 2-3, 3-6, 3-5, 3-4, 4-6, 4-5, 5-6, 0, 1, 2, 3, 4, 5, or 6.

In some variations of formula (34) described in the paragraphs above in which at least one of L1, L2, and L3 is linker (c.2), n₁ is 1-4, 1-3, 1-2, 2-4, 2-3, 3-4, 1, 2, 3, or 4.

In some variations of formula (34) described in the paragraphs above in which at least one of L1, L2, and L3 is linker (c.2), Rₐ is H and R_{b} is H. In some variations of formula (34) described in the paragraphs above in which at least one of L1, L2, and L3 is linker (c.2), Rₐ is H and R_{b} is CH₃. In some variations of formula (34) described in the paragraphs above in which at least one of L1, L2, and L3 is linker (c.2), Rₐ and R_{b} are

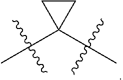 [SMILES: *C1(*)CC1]

In some variations of formula (34) described in the paragraphs above in which at least one of L1, L2, and L3 is linker (c.2), Rₐ and R_{b} are

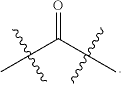 [SMILES: *C(*)=O]

In some variations of formula (34) described in the paragraphs above in which at least one of L1, L2, and L3 is linker (c.2), two of CRₐR_{b} are

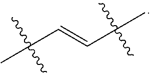 [SMILES: */C=C/*]

In some variations of formula (34) described in the paragraphs above, L1 is linker (b.1). In some variations of formula (34) described in the paragraphs above, L2 is linker (b.1). In some variations of formula (34) described in the paragraphs above, L3 is linker (b.1). In some variations of formula (34) described in the paragraphs above, L1 and L2 are linker (b.1). In some variations of formula (34) described in the paragraphs above, L1 and L3 are linker (b.1). In some variations of formula (34) described in the paragraphs above, L2 and L3 are linker (b.1). In some variations of formula (34) described in the paragraphs above, L1, L2, and L3 are linker (b.1).

In some variations of formula (34) described in the paragraphs above in which at least one of L1, L2, and L3 is linker (c.2), L is

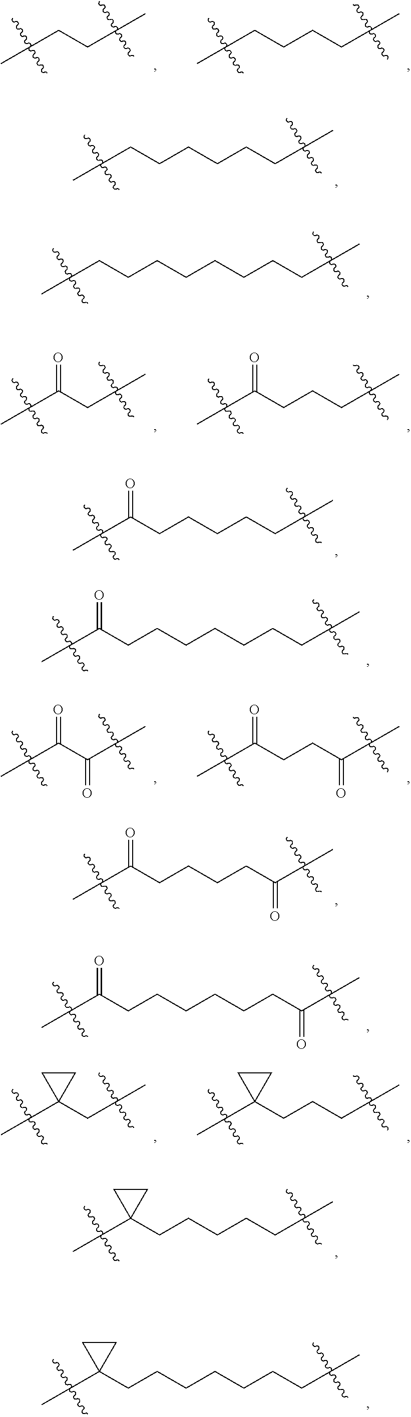

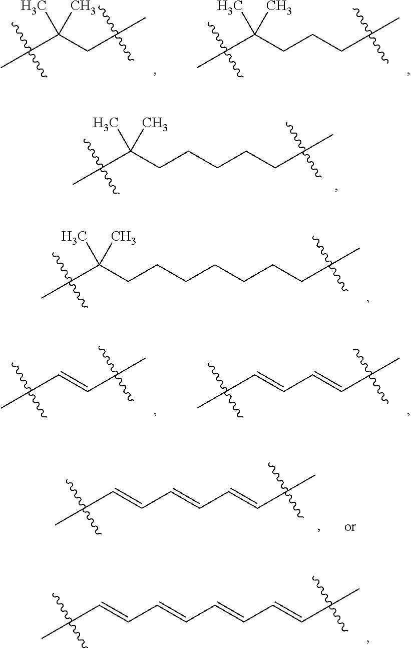

In some variations of formula (34) described in the paragraphs above, L1 is linker (c.2). In some variations of formula (34) described in the paragraphs above, L2 is linker (c.2). In some variations of formula (34) described in the paragraphs above, L3 is linker (c.2). In some variations of formula (34) described in the paragraphs above, L1 and L2 are linker (c.2). In some variations of formula (34) described in the paragraphs above, L1 and L3 are linker (c.2). In some variations of formula (34) described in the paragraphs above, L2 and L3 are linker (c.2). In some variations of formula (34) described in the paragraphs above, L1, L2, and L3 are linker (c.2).

In some variations of formula (34) described in the paragraphs above, at least one of L1, L2, and L3 is linker (d):

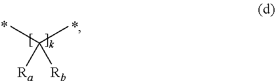

where k is 2-10; (1) Rₐ and R_{b} independently are H or CH₃, or (2) Rₐ and R_{b} are

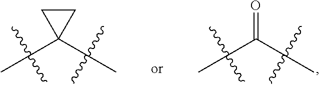

or (3) two of CRₐR_{b} are

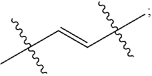 [SMILES: */C=C/*]

and * is the attachment site for the cationic dye moiety D

In some variations of formula (34) in which at least one of L1, L2, and L3 is linker (d), k is 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, 9-10, 2, 3, 4, 5, 6, 7, 8, 9, or 10.

In some variations of formula (34) described in the paragraphs above in which at least one of L1, L2, and L3 is linker (d), Rₐ is H and R_{b} is H. In some variations of formula (34) described in the paragraphs above in which at least one of L1, L2, and L3 is linker (d), Rₐ is H and R_{b} is CH₃. In some variations of formula (34) described in the paragraphs above in which at least one of L1, L2, and L3 is linker (d), Rₐ and R_{b} are

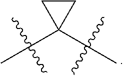 [SMILES: *C1(*)CC1]

In some variations of formula (34) described in the paragraphs above in which at least one of L1, L2, and L3 is linker (d), Rₐ and R_{b} are

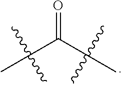 [SMILES: *C(*)=O]

In some variations of formula (34) described in the paragraphs above in which at least one of L1, L2, and L3 is linker (d), two of CRₐR_{b} are

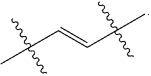 [SMILES: */C=C/*]

In some variations of formula (34) described in the paragraphs above, L1 is linker (d). In some variations of formula (34) described in the paragraphs above, L2 is linker (d). In some variations of formula (34) described in the paragraphs above, L3 is linker (d). In some variations of formula (34) described in the paragraphs above, L1 and L2 are linker (d). In some variations of formula (34) described in the paragraphs above, L1 and L3 are linker (d). In some variations of formula (34) described in the paragraphs above, L2 and L3 are linker (d). In some variations of formula (34) described in the paragraphs above, L1, L2, and L3 are linker (d).

In some variations of formula (34) described in the paragraphs above, at least one of L1, L2, and L3 is linker (e):

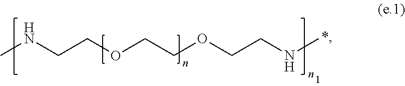

in which n is 0-6, n₁ is 1-4, and * is the attachment site for the cationic dye moiety D.

In some variations of formula (34) in which at least one of L1, L2, and L3 is linker (e.1), n is 0-6, 0-5, 0-4, 0-3, 0-2, 0-1, 1-6, 1-5, 1-4, 1-3, 1-2, 2-6, 2-5, 2-4, 2-3, 3-6, 3-5, 3-4, 4-6, 4-5, 5-6, 0, 1, 2, 3, 4, 5, or 6.

In some variations of formula (34) described in the paragraphs above in which at least one of L1, L2, and L3 is linker (e.1), n₁ is 1-4, 1-3, 1-2, 2-4, 2-3, 3-4, 1, 2, 3, or 4.

In some variations of formula (34) described in the paragraphs above, L1 is linker (e.1). In some variations of formula (34) described in the paragraphs above, L2 is linker (e.1). In some variations of formula (34) described in the paragraphs above, L3 is linker (e.1). In some variations of formula (34) described in the paragraphs above, L1 and L2 are linker (e.1). In some variations of formula (34) described in the paragraphs above, L1 and L3 are linker (e.1). In some variations of formula (34) described in the paragraphs above, L2 and L3 are linker (e.1). In some variations of formula (34) described in the paragraphs above, L1, L2, and L3 are linker (e.1).

In some variations of formula (34) described in the paragraphs above, at least one of L1, L2, and L3 is linker (f.1):

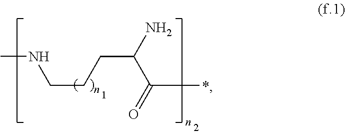

in which n₁ is 0-5, n₂ is 1-5, and * is the attachment site for the cationic dye moiety D.

In some variations of formula (34) in which at least one of L1, L2, and L3 is linker (f.1), n₁ is 0-5, 0-4, 0-3, 0-2, 0-1, 1-5, 1-4, 1-3, 1-2, 2-5, 2-4, 2-3, 3-5, 3-4, 4-5, 0, 1, 2, 3, 4, or 5.

In some variations of formula (34) described in the paragraphs above in which at least one of L1, L2, and L3 is linker (f.1), n₂ is 1-5, 1-4, 1-3, 1-2, 2-5, 2-4, 2-3, 3-5, 3-4, 4-5, 1, 2, 3, 4, or 5.

In some variations of formula (34) described in the paragraphs above, L1 is linker (f.1). In some variations of formula (34) described in the paragraphs above, L2 is linker (f.1). In some variations of formula (34) described in the paragraphs above, L3 is linker (f.1). In some variations of formula (34) described in the paragraphs above, L1 and L2 are linker (f.1). In some variations of formula (34) described in the paragraphs above, L1 and L3 are linker (f.1). In some variations of formula (34) described in the paragraphs above, L2 and L3 are linker (f.1). In some variations of formula (34) described in the paragraphs above, L1, L2, and L3 are linker (f.1).

In some variations of formula (34) described in the paragraphs above, at least one of L1, L2, and L3 is linker (f.2):

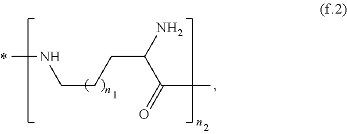

in which n₁ is 0-5, n₂ is 1-5, and * is the attachment site for the cationic dye moiety D.

In some variations of formula (34) in which at least one of L1, L2, and L3 is linker (f.2), n₁ is 0-5, 0-4, 0-3, 0-2, 0-1, 1-5, 1-4, 1-3, 1-2, 2-5, 2-4, 2-3, 3-5, 3-4, 4-5, 0, 1, 2, 3, 4, or 5.

In some variations of formula (34) described in the paragraphs above in which at least one of L1, L2, and L3 is linker (f.2), n₂ is 1-5, 1-4, 1-3, 1-2, 2-5, 2-4, 2-3, 3-5, 3-4, 4-5, 1, 2, 3, 4, or 5.

In some variations of formula (34) described in the paragraphs above, L1 is linker (f.2). In some variations of formula (34) described in the paragraphs above, L2 is linker (f.2). In some variations of formula (34) described in the paragraphs above, L3 is linker (f.2). In some variations of formula (34) described in the paragraphs above, L1 and L2 are linker (f.2). In some variations of formula (34) described in the paragraphs above, L1 and L3 are linker (f.2). In some variations of formula (34) described in the paragraphs above, L2 and L3 are linker (f.2). In some variations of formula (34) described in the paragraphs above, L1, L2, and L3 are linker (f.2).

In some variations of formula (34) described in the paragraphs above, at least one of L1, L2, and L3 is linker (g.1):

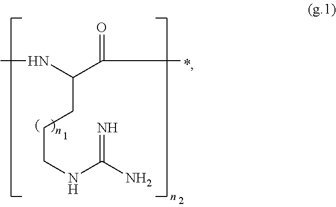

in which n₁ is 0-5, n₂ is 1-5, and * is the attachment site for the cationic dye moiety D.

In some variations of formula (34) in which at least one of L1, L2, and L3 is linker (g.1), n₁ is 0-5, 0-4, 0-3, 0-2, 0-1, 1-5, 1-4, 1-3, 1-2, 2-5, 2-4, 2-3, 3-5, 3-4, 4-5, 0, 1, 2, 3, 4, or 5.

In some variations of formula (34) described in the paragraphs above in which at least one of L1, L2, and L3 is linker (g.1), n₂ is 1-5, 1-4, 1-3, 1-2, 2-5, 2-4, 2-3, 3-5, 3-4, 4-5, 1, 2, 3, 4, or 5.

In some variations of formula (34) described in the paragraphs above, L1 is linker (g.1). In some variations of formula (34) described in the paragraphs above, L2 is linker (g.1). In some variations of formula (34) described in the paragraphs above, L3 is linker (g.1). In some variations of formula (34) described in the paragraphs above, L1 and L2 are linker (g.1). In some variations of formula (34) described in the paragraphs above, L1 and L3 are linker (g.1). In some variations of formula (34) described in the paragraphs above, L2 and L3 are linker (g.1). In some variations of formula (34) described in the paragraphs above, L1, L2, and L3 are linker (g.1).

In some variations of formula (34) described in the paragraphs above, at least one of L1, L2, and L3 is linker (g.2):

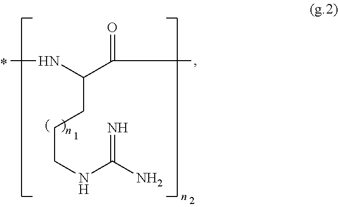

in which n₁ is 0-5, n₂ is 1-5, and * is the attachment site for the cationic dye moiety D.

In some variations of formula (34) in which at least one of L1, L2, and L3 is linker (g.2), n₁ is 0-5, 0-4, 0-3, 0-2, 0-1, 1-5, 1-4, 1-3, 1-2, 2-5, 2-4, 2-3, 3-5, 3-4, 4-5, 0, 1, 2, 3, 4, or 5.

In some variations of formula (34) described in the paragraphs above in which at least one of L1, L2, and L3 is linker (g.2), n₂ is 1-5, 1-4, 1-3, 1-2, 2-5, 2-4, 2-3, 3-5, 3-4, 4-5, 1, 2, 3, 4, or 5.

In some variations of formula (34) described in the paragraphs above, L1 is linker (g.2). In some variations of formula (34) described in the paragraphs above, L2 is linker (g.2). In some variations of formula (34) described in the paragraphs above, L3 is linker (g.2). In some variations of formula (34) described in the paragraphs above, L1 and L2 are linker (g.2). In some variations of formula (34) described in the paragraphs above, L1 and L3 are linker (g.2). In some variations of formula (34) described in the paragraphs above, L2 and L3 are linker (g.2). In some variations of formula (34) described in the paragraphs above, L1, L2, and L3 are linker (g.2).

In some variations of formula (34) described in the paragraphs above, at least one of L1, L2, and L3 is linker (h.1):

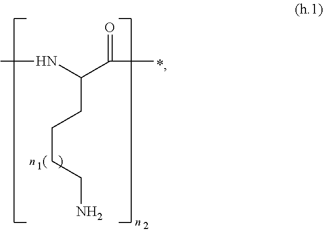

in which n₁ is 0-5, n₂ is 1-5, and * is the attachment site for the cationic dye moiety D.

In some variations of formula (34) in which at least one of L1, L2, and L3 is linker (h.1), n₁ is 0-5, 0-4, 0-3, 0-2, 0-1, 1-5, 1-4, 1-3, 1-2, 2-5, 2-4, 2-3, 3-5, 3-4, 4-5, 0, 1, 2, 3, 4, or 5.

In some variations of formula (34) described in the paragraphs above in which at least one of L1, L2, and L3 is linker (h.1), n₂ is 1-5, 1-4, 1-3, 1-2, 2-5, 2-4, 2-3, 3-5, 3-4, 4-5, 1, 2, 3, 4, or 5.

In some variations of formula (34) described in the paragraphs above, L1 is linker (h.1). In some variations of formula (34) described in the paragraphs above, L2 is linker (h.1). In some variations of formula (34) described in the paragraphs above, L3 is linker (h.1). In some variations of formula (34) described in the paragraphs above, L1 and L2 are linker (h.1). In some variations of formula (34) described in the paragraphs above, L1 and L3 are linker (h.1). In some variations of formula (34) described in the paragraphs above, L2 and L3 are linker (h.1). In some variations of formula (34) described in the paragraphs above, L1, L2, and L3 are linker (h.1).

In some variations of formula (34) described in the paragraphs above, at least one of L1, L2, and L3 is linker (h.2):

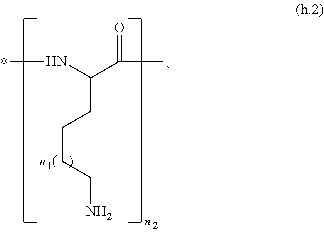

in which n₁ is 0-5, n₂ is 1-5, and * is the attachment site for the cationic dye moiety D.

In some variations of formula (34) in which at least one of L1, L2, and L3 is linker (h.2), n₁ is 0-5, 0-4, 0-3, 0-2, 0-1, 1-5, 1-4, 1-3, 1-2, 2-5, 2-4, 2-3, 3-5, 3-4, 4-5, 0, 1, 2, 3, 4, or 5.

In some variations of formula (34) described in the paragraphs above in which at least one of L1, L2, and L3 is linker (h.2), n₂ is 1-5, 1-4, 1-3, 1-2, 2-5, 2-4, 2-3, 3-5, 3-4, 4-5, 1, 2, 3, 4, or 5.

In some variations of formula (34) described in the paragraphs above, L1 is linker (h.2). In some variations of formula (34) described in the paragraphs above, L2 is linker (h.2). In some variations of formula (34) described in the paragraphs above, L3 is linker (h.2). In some variations of formula (34) described in the paragraphs above, L1 and L2 are linker (h.2). In some variations of formula (34) described in the paragraphs above, L1 and L3 are linker (h.2). In some variations of formula (34) described in the paragraphs above, L2 and L3 are linker (h.2). In some variations of formula (34) described in the paragraphs above, L1, L2, and L3 are linker (h.2).

In some variations of formula (34) described in the paragraphs above, at least one of L1, L2, and L3 is linker (i.1):

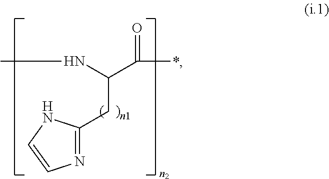

in which n₁ and n₂ independently are 1-5 and * is the attachment site for the cationic dye moiety D.

In some variations of formula (34) described in the paragraphs above, at least one of L1, L2, and L3 is linker (i.1), n₁ is 1-5, 1-4, 1-3, 1-2, 2-5, 2-4, 2-3, 3-5, 3-4, 4-5, 1, 2, 3, 4, or 5.

In some variations of formula (34) described in the paragraphs above in which at least one of L1, L2, and L3 is linker (i.1), n₁ is 1-5, 1-4, 1-3, 1-2, 2-5, 2-4, 2-3, 3-5, 3-4, 4-5, 1, 2, 3, 4, or 5.

In some variations of formula (34) described in the paragraphs above, L1 is linker (i.1). In some variations of formula (34) described in the paragraphs above, L2 is linker (i.1). In some variations of formula (34) described in the paragraphs above, L3 is linker (i.1). In some variations of formula (34) described in the paragraphs above, L1 and L2 are linker (i.1). In some variations of formula (34) described in the paragraphs above, L1 and L3 are linker (i.1). In some variations of formula (34) described in the paragraphs above, L2 and L3 are linker (i.1). In some variations of formula (34) described in the paragraphs above, L1, L2, and L3 are linker (i.1).

In some variations of formula (34) described in the paragraphs above, at least one of L1, L2, and L3 is linker (i.2):

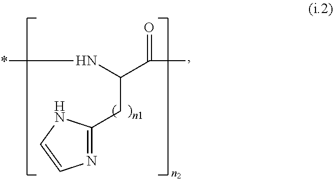

in which n₁ and n₂ independently are 1-5 and * is the attachment site for the cationic dye moiety D.

In some variations of formula (34) described in the paragraphs above, at least one of L1, L2, and L3 is linker (i.2), n₁ is 1-5, 1-4, 1-3, 1-2, 2-5, 2-4, 2-3, 3-5, 3-4, 4-5, 1, 2, 3, 4, or 5.

In some variations of formula (34) described in the paragraphs above in which at least one of L1, L2, and L3 is linker (i.2), n₁ is 1-5, 1-4, 1-3, 1-2, 2-5, 2-4, 2-3, 3-5, 3-4, 4-5, 1, 2, 3, 4, or 5.

In some variations of formula (34) described in the paragraphs above, L1 is linker (i.2). In some variations of formula (34) described in the paragraphs above, L2 is linker (i.2). In some variations of formula (34) described in the paragraphs above, L3 is linker (i.2). In some variations of formula (34) described in the paragraphs above, L1 and L2 are linker (i.2). In some variations of formula (34) described in the paragraphs above, L1 and L3 are linker (i.2). In some variations of formula (34) described in the paragraphs above, L2 and L3 are linker (i.2). In some variations of formula (34) described in the paragraphs above, L1, L2, and L3 are linker (i.2).

In some variations of formula (34) described in the paragraphs above, at least one of L1, L2, and L3 is linker (j.1):

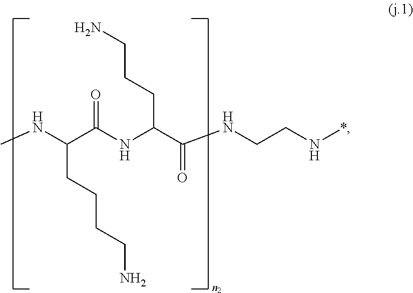

in which n₂ is 1-5 and * is the attachment site for the cationic dye moiety D.

In some variations of formula (34) in which at least one of L1, L2, and L3 is linker (j.1), n₂ is 1-5, 1-4, 1-3, 1-2, 2-5, 2-4, 2-3, 3-5, 3-4, 4-5, 1, 2, 3, 4, or 5.

In some variations of formula (34) described in the paragraphs above, L1 is linker (j.1). In some variations of formula (34) described in the paragraphs above, L2 is linker (j.1). In some variations of formula (34) described in the paragraphs above, L3 is linker (j.1). In some variations of formula (34) described in the paragraphs above, L1 and L2 are linker (j.1). In some variations of formula (34) described in the paragraphs above, L1 and L3 are linker (j.1). In some variations of formula (34) described in the paragraphs above, L2 and L3 are linker (j.1). In some variations of formula (34) described in the paragraphs above, L1, L2, and L3 are linker (j.1).

In some variations of formula (34) described in the paragraphs above, at least one of L1, L2, and L3 is linker (j.2):

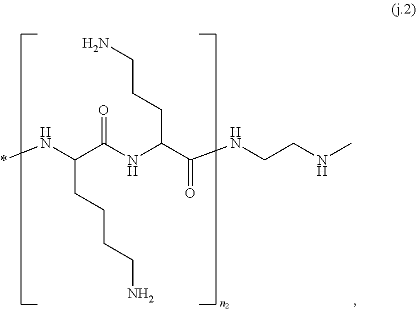

in which n₂ is 1-5 and * is the attachment site for the cationic dye moiety D.

In some variations of formula (34) in which at least one of L1, L2, and L3 is linker (j.2), n₂ is 1-5, 1-4, 1-3, 1-2, 2-5, 2-4, 2-3, 3-5, 3-4, 4-5, 1, 2, 3, 4, or 5.

In some variations of formula (34) described in the paragraphs above, L1 is linker (j.2). In some variations of formula (34) described in the paragraphs above, L2 is linker (j.2). In some variations of formula (34) described in the paragraphs above, L3 is linker (j.2). In some variations of formula (34) described in the paragraphs above, L1 and L2 are linker (j.2). In some variations of formula (34) described in the paragraphs above, L1 and L3 are linker (j.2). In some variations of formula (34) described in the paragraphs above, L2 and L3 are linker (j.2). In some variations of formula (34) described in the paragraphs above, L1, L2, and L3 are linker (j.2).

In some variations of formula (34) described in the paragraphs above, at least one of L1, L2, and L3 is linker (k):

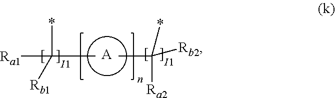

in which l₁ and l₂ independently are 1-4, n is 1-4; ring A is aryl, heteroaryl, cycloalkyl, or heterocyclyl; (1) Rₐ₁ and R_{b1} independently are H or CH₃ or (2) Rₐ₁ and R_{b1} independently are

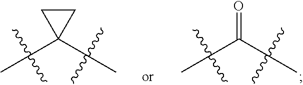

or (3) two of CRₐ₁R_{b1} are

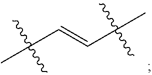 [SMILES: */C=C/*]

(1) Rₐ₂ and R_{b2} independently are H or CH₃ or (2) Rₐ₂ and R_{b2} independently are

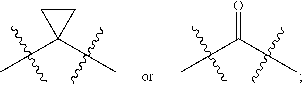

or (3) two of CRₐ₂R_{b2} are

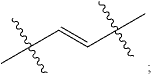 [SMILES: */C=C/*]

and * is the attachment site for the cationic dye moiety D.

In some variations of formula (34) in which at least one of L1, L2, and L3 is linker (k), l₁ is 1-4, 1-3, 1-3, 1-2, 2-4, 2-3, 3-4, 1, 2, 3, or 4.

In some variations of formula (34) described in the paragraphs above in which at least one of L1, L2, and L3 is linker (k), l₂ is 1-4, 1-3, 1-3, 1-2, 2-4, 2-3, 3-4, 1, 2, 3, or 4.

In some variations of formula (34) described in the paragraphs above in which at least one of L1, L2, and L3 is linker (k), Rₐ₁ is H and R_{b1} is H. In some variations of formula (34) described in the paragraphs above in which at least one of L1, L2, and L3 is linker (k), Rₐ₁ is H and R_{b1} is CH₃. In some variations of formula (34) described in the paragraphs above in which at least one of L1, L2, and L3 is linker (k), Rₐ₁ and R_{b1} are

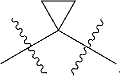 [SMILES: *C1(*)CC1]

In some variations of formula (34) described in the paragraphs above in which at least one of L1, L2, and L3 is linker (k), Rₐ₁ and R_{b1} are

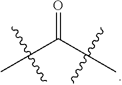 [SMILES: *C(*)=O]

In some variations of formula (34) described in the paragraphs above in which at least one of L1, L2, and L3 is linker (k), two of CRₐ₁R_{b1} are

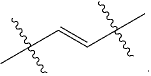 [SMILES: */C=C/*]

In some variations of formula (34) described in the paragraphs above in which at least one of L1, L2, and L3 is linker (k), Rₐ₂ is H and R_{b2} is H. In some variations of formula (34) described in the paragraphs above in which at least one of L1, L2, and L3 is linker (k), Rₐ₂ is H and R_{b2} is CH₃. In some variations of formula (34) described in the paragraphs above in which at least one of L1, L2, and L3 is linker (k), Rₐ₂ and R_{b2} are

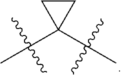 [SMILES: *C1(*)CC1]

In some variations of formula (34) described in the paragraphs above in which at least one of L1, L2, and L3 is linker (k), Rₐ₂ and R_{b2} are

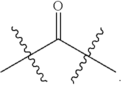 [SMILES: *C(*)=O]

In some variations of formula (34) described in the paragraphs above in which at least one of L1, L2, and L3 is linker (k), two of CRₐ₂R_{b2} are

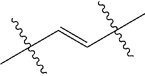 [SMILES: */C=C/*]

In some variations of formula (34) described in the paragraphs above in which at least one of L1, L2, and L3 is linker (k), ring A is

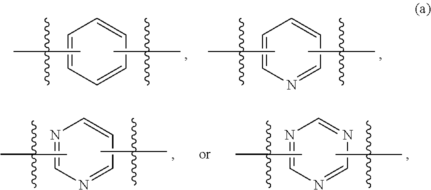

optionally substituted with halo or C1-C6 linear or branched alkyl;

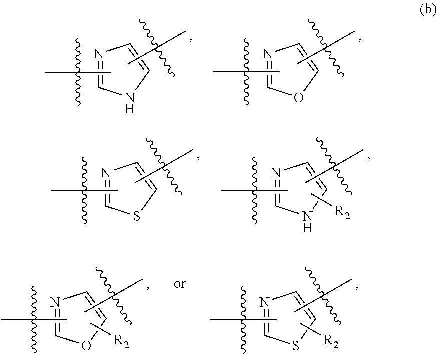

optionally substituted with halo or C1-C6 linear or branched alkyl; or

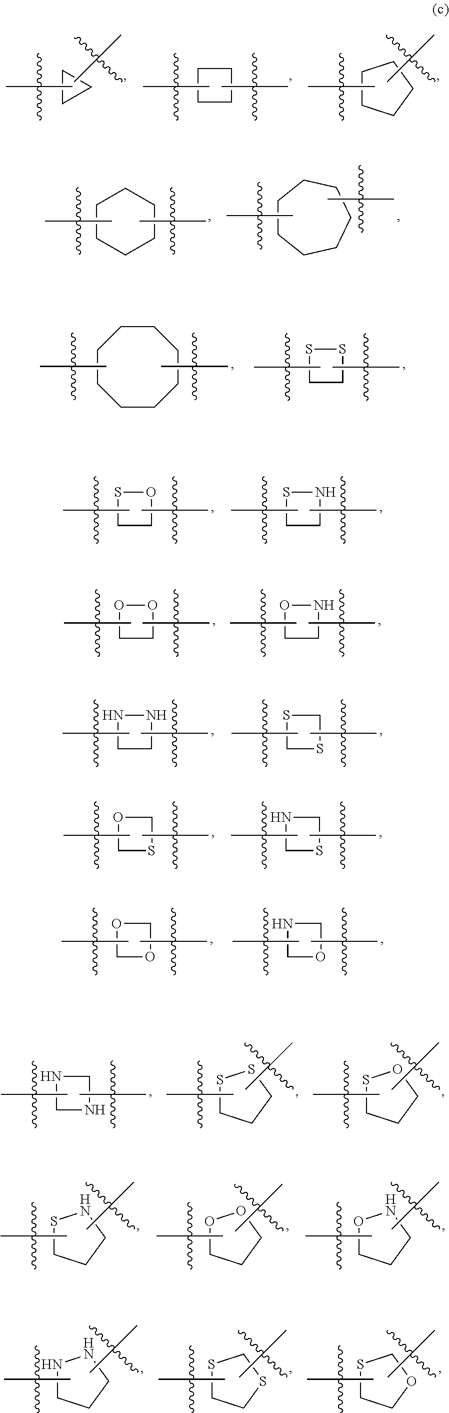

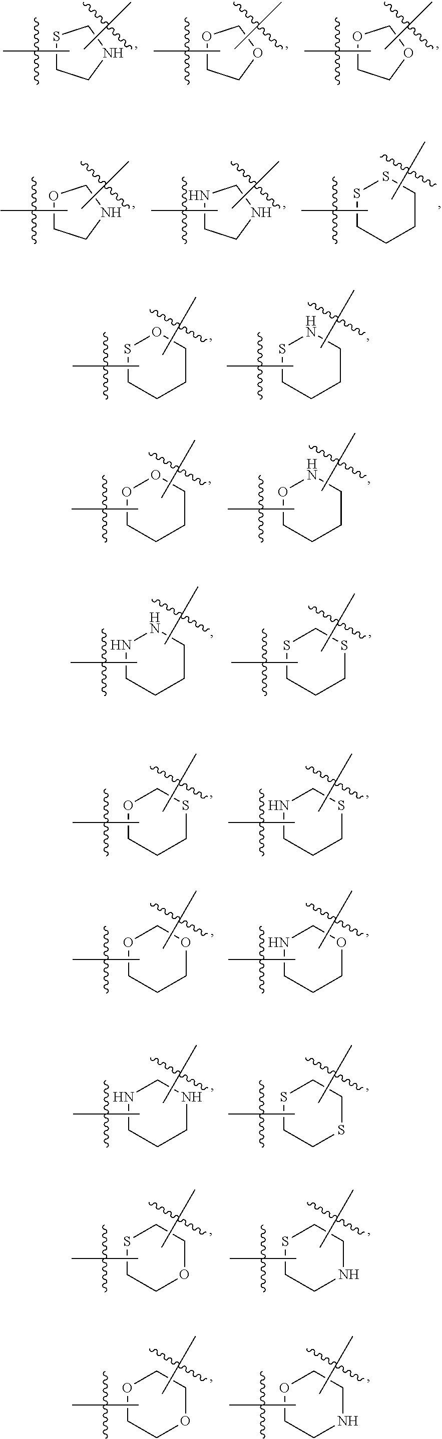

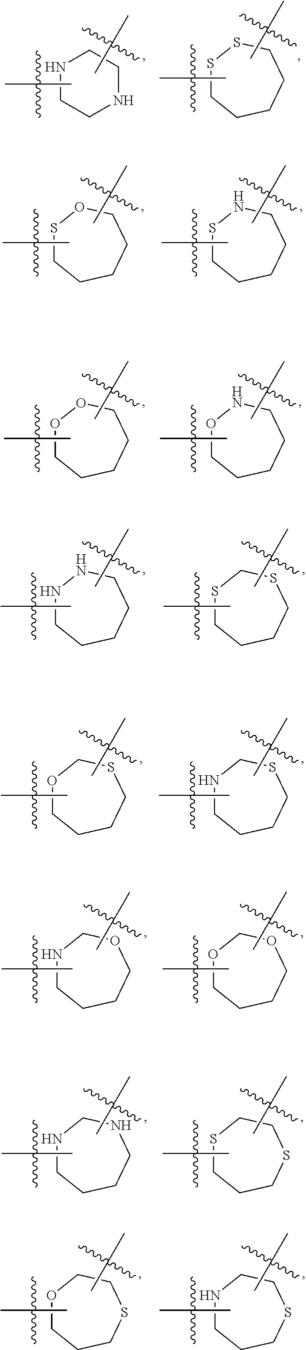

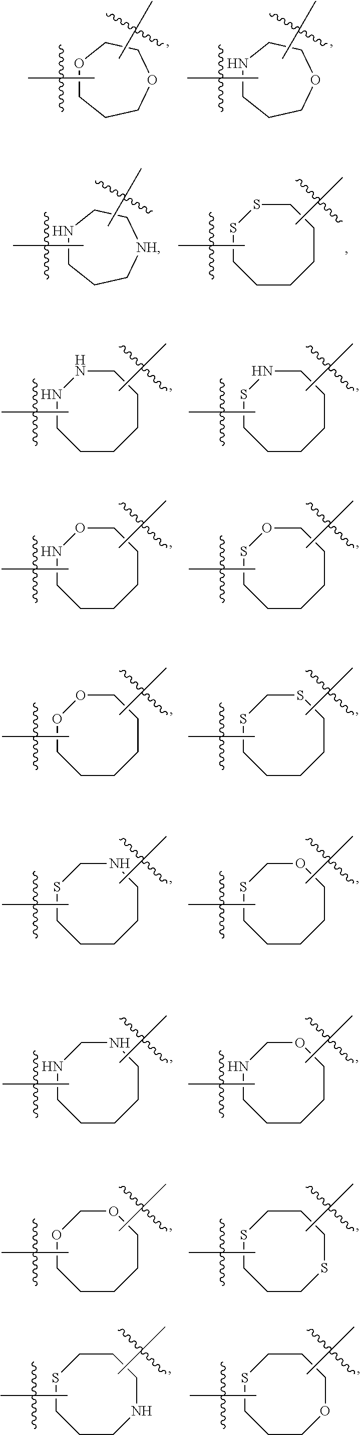

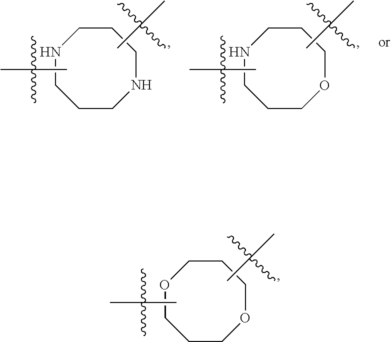

optionally substituted with halo or C1-C6 linear or branched alkyl.

In some variations of formula (34) described in the paragraphs above in which L is linker (k), ring A is substituted with halo. In some variations, the halo is F, Br, I, or Cl.

In some variations of formula (34) described in the paragraphs above in which L is linker (k), ring A is substituted with C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (34) described in the paragraphs above, L1 is linker (k). In some variations of formula (34) described in the paragraphs above, L2 is linker (k). In some variations of formula (34) described in the paragraphs above, L3 is linker (k). In some variations of formula (34) described in the paragraphs above, L1 and L2 are linker (k). In some variations of formula (34) described in the paragraphs above, L1 and L3 are linker (k). In some variations of formula (34) described in the paragraphs above, L2 and L3 are linker (k). In some variations of formula (34) described in the paragraphs above, L1, L2, and L3 are linker (k).

In some variations of formula (34) described in the paragraphs above, at least one of L1, L2, and L3 is linker (l.1):

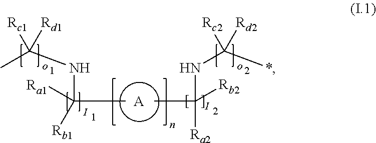

in which l₁, l₂, n, o₁, and o₂ independently are 1-4; ring A is aryl, heteroaryl, cycloalkyl, or heterocyclyl; for each independent instance of Rₐ₁ and R_{b1}, Rₐ₁ and R_{b1} (1) independently are H or CH₃, or (2) Rₐ₁ and R_{b1} independently are

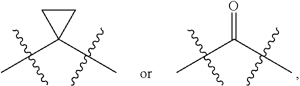

or (3) two of CRₐ₁R_{b1} are

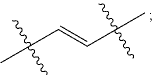 [SMILES: */C=C/*]

for each independent instance of Rₐ₂ and R_{b2}, Rₐ₂ and R_{b2} (1) independently are H or CH₃, or (2) Rₐ₂ and R_{b2} independently are

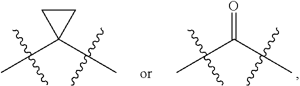

or (3) two of CRₐ₂R_{b2} are

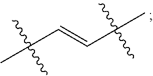 [SMILES: */C=C/*]

for each independent instance of R_{c1} and R_{d1}, R_{c1} and R_{d1} (1) independently are H or CH₃, or (2) R_{d} and Rₐ₁ independently are

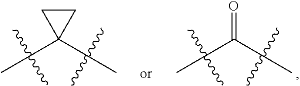

or (3) two of CR_{c1}R_{d1} are

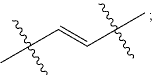 [SMILES: */C=C/*]

for each independent instance of R_{c2} and R_{d2}, R_{c2} and R_{d2} (1) independently are H or CH₃, or (2) R_{c2} and R_{d2} independently are

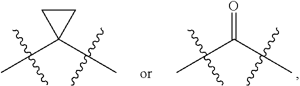

or (3) two of CR_{c2}R_{d2} are

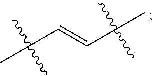 [SMILES: */C=C/*]

and * is the attachment site for the cationic dye moiety D.

In some variations of formula (34) in which at least one of L1, L2, and L3 is linker (l.1), l₁ is 1-4, 1-3, 1-3, 1-2, 2-4, 2-3, 3-4, 1, 2, 3, or 4.

In some variations of formula (34) described in the paragraphs above in which at least one of L1, L2, and L3 is linker (l.1), l₂ is 1-4, 1-3, 1-3, 1-2, 2-4, 2-3, 3-4, 1, 2, 3, or 4.

In some variations of formula (34) described in the paragraphs above in which at least one of L1, L2, and L3 is linker (l.1), o₁ is 1-4, 1-3, 1-3, 1-2, 2-4, 2-3, 3-4, 1, 2, 3, or 4.

In some variations of formula (34) described in the paragraphs above in which at least one of L1, L2, and L3 is linker (l.1), o₂ is 1-4, 1-3, 1-3, 1-2, 2-4, 2-3, 3-4, 1, 2, 3, or 4.

In some variations of formula (34) described in the paragraphs above in which at least one of L1, L2, and L3 is linker (l.1), Rₐ₁ is H and R_{b1} is H. In some variations of formula (34) described in the paragraphs above in which at least one of L1, L2, and L3 is linker (l.1), Rₐ₁ is H and R_{b1} is CH₃. In some variations of formula (34) described in the paragraphs above in which at least one of L1, L2, and L3 is linker (l.1), Rₐ₁ and R_{b1} are

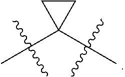 [SMILES: *C1(*)CC1]

In some variations of formula (34) described in the paragraphs above in which at least one of L1, L2, and L3 is linker (l.1), Rₐ₁ and R_{b1} are

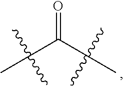 [SMILES: *C(*)=O]

In some variations of formula (34) described in the paragraphs above in which at least one of L1, L2, and L3 is linker (l.1), two of CRₐ₁R_{b1} are

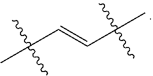 [SMILES: */C=C/*]

In some variations of formula (34) described in the paragraphs above in which at least one of L1, L2, and L3 is linker (l.1), Rₐ₂ is H and R_{b2} is H. In some variations of formula (34) described in the paragraphs above in which at least one of L1, L2, and L3 is linker (l.1), Rₐ₂ is H and R_{b2} is CH₃. In some variations of formula (34) described in the paragraphs above in which at least one of L1, L2, and L3 is linker (l.1), Rₐ₂ and R_{b2} are

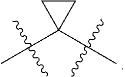 [SMILES: *C1(*)CC1]

In some variations of formula (34) described in the paragraphs above in which at least one of L1, L2, and L3 is linker (l.1), Rₐ₂ and R_{b2} are

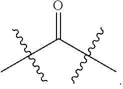 [SMILES: *C(*)=O]

In some variations of formula (34) described in the paragraphs above in which at least one of L1, L2, and L3 is linker (l.1), two of CRₐ₂R_{b2} are

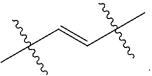 [SMILES: */C=C/*]

In some variations of formula (34) described in the paragraphs above in which at least one of L1, L2, L3, and L4 is linker (l.1), R_{c1} is H and R_{d1} is H. In some variations of formula (34) described in the paragraphs above in which at least one of L1, L2, L3, and L4 is linker (l.1), R_{c1} is H and R_{d1} is CH₃. In some variations of formula (34) described in the paragraphs above in which at least one of L1, L2, L3, and L4 is linker (l.1), R_{c1} and R_{d1} are

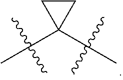 [SMILES: *C1(*)CC1]

In some variations of formula (34) described in the paragraphs above in which at least one of L1, L2, L3, and L4 is linker (l.1), R_{c1} and R_{d1} are

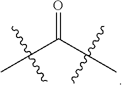 [SMILES: *C(*)=O]

In some variations of formula (34) described in the paragraphs above in which at least one of L1, L2, L3, and L4 is linker (l.1), two of CR_{c1}R_{d1} are

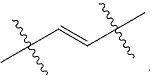 [SMILES: */C=C/*]

In some variations of formula (34) described in the paragraphs above in which at least one of L1, L2, L3, and L4 is linker (l.1), R_{c2} is H and R_{d2} is H. In some variations of formula (34) described in the paragraphs above in which at least one of L1, L2, L3, and L4 is linker (l.1), R_{c2} is H and R_{d2} is CH₃. In some variations of formula (34) described in the paragraphs above in which at least one of L1, L2, L3, and L4 is linker (l.1), R_{c2} and R_{d2} are

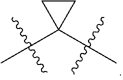 [SMILES: *C1(*)CC1]

In some variations of formula (34) described in the paragraphs above in which at least one of L1, L2, L3, and L4 is linker (l.1), R_{c2} and R_{d2} are

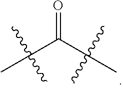 [SMILES: *C(*)=O]

In some variations of formula (34) described in the paragraphs above in which at least one of L1, L2, L3, and L4 is linker (l.1), two of CR_{c2}R_{d2} are

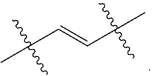 [SMILES: */C=C/*]

In some variations of formula (34) described in the paragraphs above in which at least one of L1, L2, and L3 is linker (l.1), ring A is

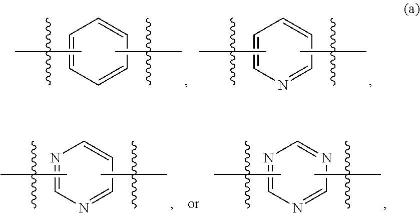

optionally substituted with halo or C1-C6 linear or branched alkyl;

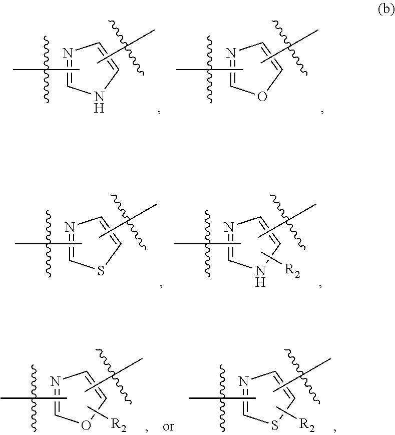

optionally substituted with halo or C1-C6 linear or branched alkyl; or

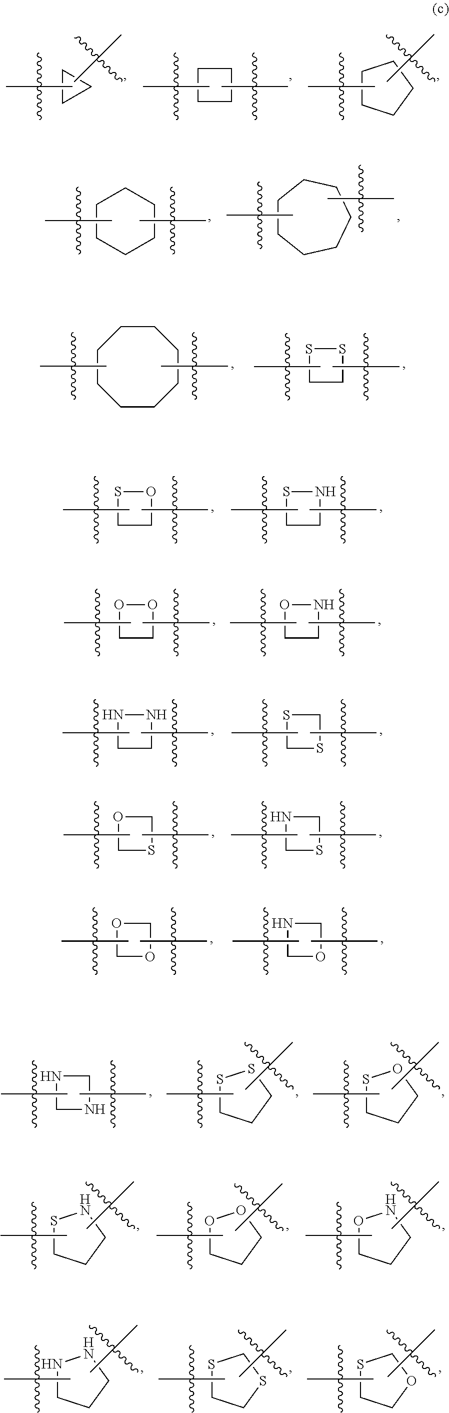

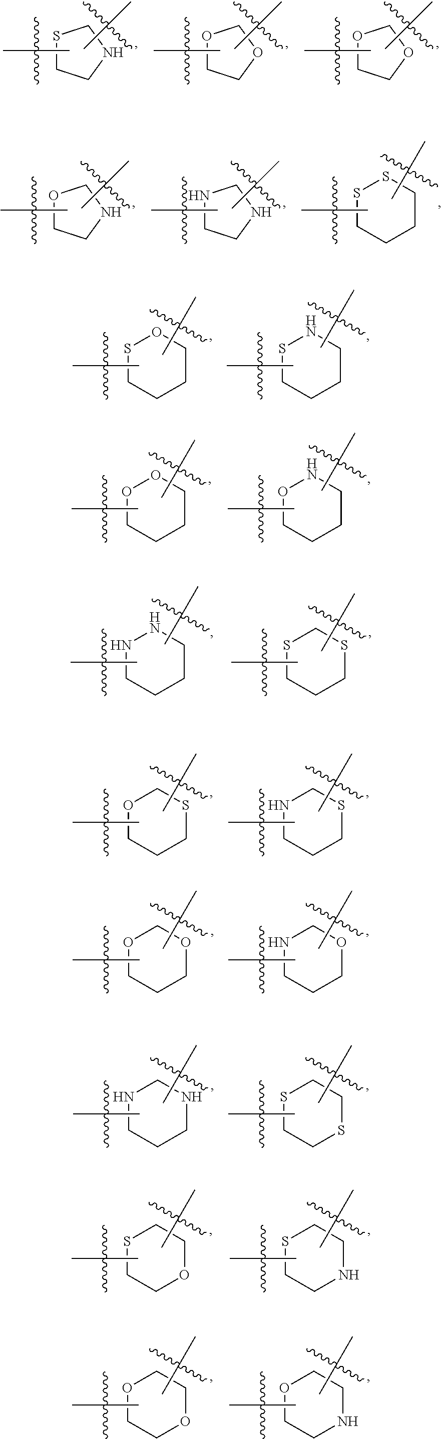

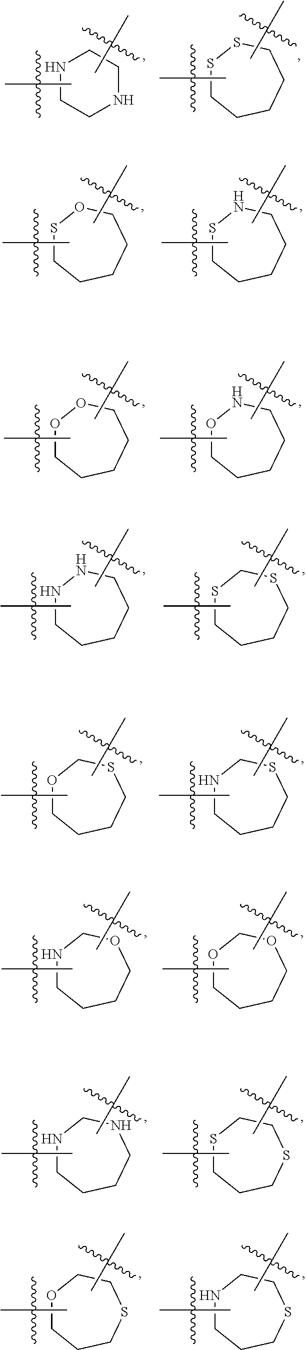

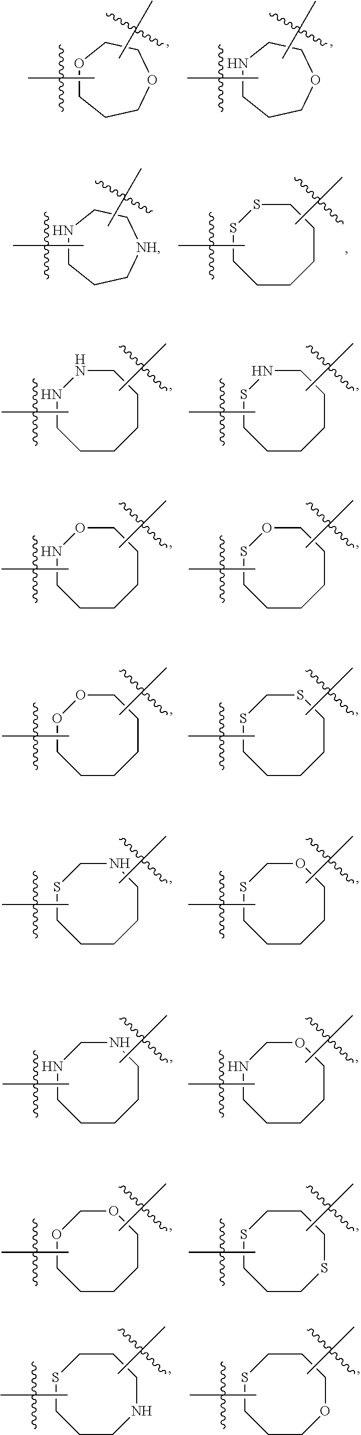

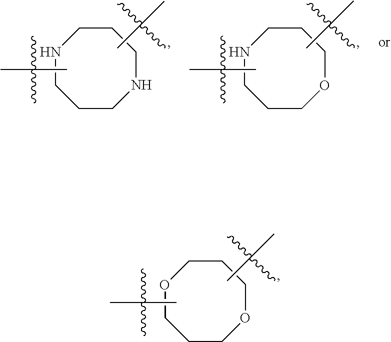

optionally substituted with halo or C1-C6 linear or branched alkyl.

In some variations of formula (34) described in the paragraphs above in which L is linker (l.1), ring A is substituted with halo. In some variations, the halo is F, Br, I, or Cl.

In some variations of formula (34) described in the paragraphs above in which L is linker (l.1), ring A is substituted with C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (34) described in the paragraphs above in which at least one of L1, L2, and L3 is linker (l.1), n is 1-4, 1-3, 1-3, 1-2, 2-4, 2-3, 3-4, 1, 2, 3, or 4.

In some variations of formula (34) described in the paragraphs above, L1 is linker (l.1). In some variations of formula (34) described in the paragraphs above, L2 is linker (l.1). In some variations of formula (34) described in the paragraphs above, L3 is linker (l.1). In some variations of formula (34) described in the paragraphs above, L1 and L2 are linker (l.1). In some variations of formula (34) described in the paragraphs above, L1 and L3 are linker (l.1). In some variations of formula (34) described in the paragraphs above, L2 and L3 are linker (l.1). In some variations of formula (34) described in the paragraphs above, L1, L2, and L3 are linker (l.1).

In some variations of formula (34) described in the paragraphs above, at least one of L1, L2, and L3 is linker (l.2):

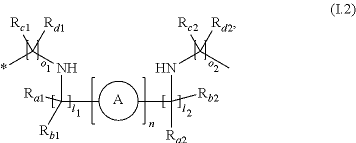

in which l₁, l₂, n, o₁, and o₂ independently are 1-4; ring A is aryl, heteroaryl, cycloalkyl, or heterocyclyl; for each independent instance of Rₐ₁ and R_{b1}, Rₐ₁ and R_{b1} (1) independently are H or CH₃, or (2) Rₐ₁ and R_{b1} independently are

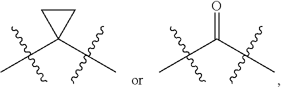

or (3) two of CRₐ₁R_{b1} are

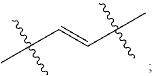 [SMILES: */C=C/*]

for each independent instance of Rₐ₂ and R_{b2}, Rₐ₂ and R_{b2} (1) independently are H or CH₃, or (2) Rₐ₂ and R_{b2} independently are

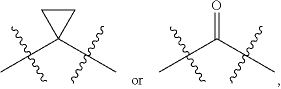

or (3) two of CRₐ₂R_{b2} are

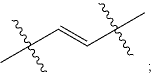 [SMILES: */C=C/*]

for each independent instance of R_{c1} and R_{d1}, R_{c1} and R_{d1} (1) independently are H or CH₃, or (2) R_{d} and Rₐ₁ independently are

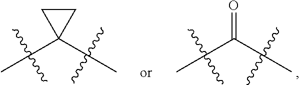

or (3) two of CR_{c1}R_{d1} are

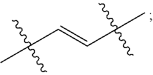 [SMILES: */C=C/*]

for each independent instance of R_{c2} and R_{d2}, R_{c2} and R_{d2} (1) independently are H or CH₃, or (2) R_{c2} and R_{d2} independently are

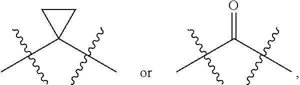

or (3) two of CR_{c2}R_{d2} are

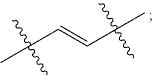 [SMILES: */C=C/*]

and * is the attachment site for the cationic dye moiety D.

In some variations of formula (34) in which at least one of L1, L2, and L3 is linker (l.2), l₁ is 1-4, 1-3, 1-3, 1-2, 2-4, 2-3, 3-4, 1, 2, 3, or 4.

In some variations of formula (34) described in the paragraphs above in which at least one of L1, L2, and L3 is linker (l.2), l₂ is 1-4, 1-3, 1-3, 1-2, 2-4, 2-3, 3-4, 1, 2, 3, or 4.

In some variations of formula (34) described in the paragraphs above in which at least one of L1, L2, and L3 is linker (l.2), o₁ is 1-4, 1-3, 1-3, 1-2, 2-4, 2-3, 3-4, 1, 2, 3, or 4.

In some variations of formula (34) described in the paragraphs above in which at least one of L1, L2, and L3 is linker (l.2), o₂ is 1-4, 1-3, 1-3, 1-2, 2-4, 2-3, 3-4, 1, 2, 3, or 4.

In some variations of formula (34) described in the paragraphs above in which at least one of L1, L2, and L3 is linker (l.2), Rₐ₁ is H and R_{b1} is H. In some variations of formula (34) described in the paragraphs above in which at least one of L1, L2, and L3 is linker (l.2), Rₐ₁ is H and R_{b1} is CH₃. In some variations of formula (34) described in the paragraphs above in which at least one of L1, L2, and L3 is linker (l.2), Rₐ₁ and R_{b1} are

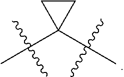 [SMILES: *C1(*)CC1]

In some variations of formula (34) described in the paragraphs above in which at least one of L1, L2, and L3 is linker (l.2), Rₐ₁ and R_{b1} are

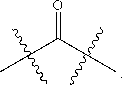 [SMILES: *C(*)=O]

In some variations of formula (34) described in the paragraphs above in which at least one of L1, L2, and L3 is linker (l.2), two of CRₐ₁R_{b1} are

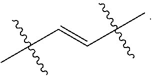 [SMILES: */C=C/*]

In some variations of formula (34) described in the paragraphs above in which at least one of L1, L2, and L3 is linker (l.2), Rₐ₂ is H and R_{b2} is H. In some variations of formula (34) described in the paragraphs above in which at least one of L1, L2, and L3 is linker (l.2), Rₐ₂ is H and R_{b2} is CH₃. In some variations of formula (34) described in the paragraphs above in which at least one of L1, L2, and L3 is linker (l.2), Rₐ₂ and R_{b2} are

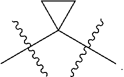 [SMILES: *C1(*)CC1]

In some variations of formula (34) described in the paragraphs above in which at least one of L1, L2, and L3 is linker (l.2), Rₐ₂ and R_{b2} are

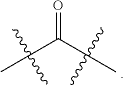 [SMILES: *C(*)=O]

In some variations of formula (34) described in the paragraphs above in which at least one of L1, L2, and L3 is linker (l.2), two of CRₐ₂R_{b2} are

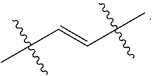 [SMILES: */C=C/*]

In some variations of formula (34) described in the paragraphs above in which at least one of L1, L2, L3, and L4 is linker (l.2), R_{c1} is H and R_{d1} is H. In some variations of formula (34) described in the paragraphs above in which at least one of L1, L2, L3, and L4 is linker (l.2), R_{c1} is H and R_{d1} is CH₃. In some variations of formula (34) described in the paragraphs above in which at least one of L1, L2, L3, and L4 is linker (l.2), R_{c1} and R_{d1} are

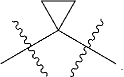 [SMILES: *C1(*)CC1]

In some variations of formula (34) described in the paragraphs above in which at least one of L1, L2, L3, and L4 is linker (l.2), R_{c1} and R_{d1} are

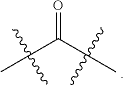 [SMILES: *C(*)=O]

In some variations of formula (34) described in the paragraphs above in which at least one of L1, L2, L3, and L4 is linker (l.2), two of CR_{c1}R_{d1} are

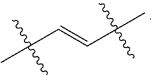 [SMILES: */C=C/*]

In some variations of formula (34) described in the paragraphs above in which at least one of L1, L2, L3, and L4 is linker (l.2), R_{c2} is H and R_{d2} is H. In some variations of formula (34) described in the paragraphs above in which at least one of L1, L2, L3, and L4 is linker (l.2), R_{c2} is H and R_{d2} is CH₃. In some variations of formula (34) described in the paragraphs above in which at least one of L1, L2, L3, and L4 is linker (l.2), R_{c2} and R_{d2} are

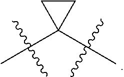 [SMILES: *C1(*)CC1]

In some variations of formula (34) described in the paragraphs above in which at least one of L1, L2, L3, and L4 is linker (l.2), R_{c2} and R_{d2} are

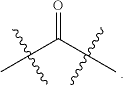 [SMILES: *C(*)=O]

In some variations of formula (34) described in the paragraphs above in which at least one of L1, L2, L3, and L4 is linker (l.2), two of CR_{c2}R_{d2} are

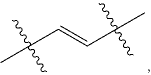 [SMILES: */C=C/*]

In some variations of formula (34) described in the paragraphs above in which at least one of L1, L2, and L3 is linker (l.2), ring A is

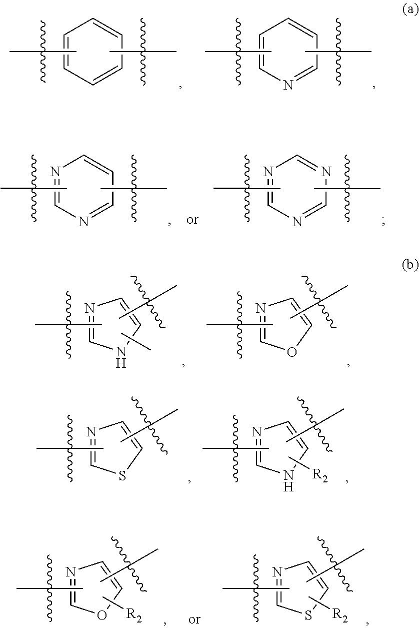

wherein R₂ is C1-C6 linear or branched alkyl, aryl, or a five-membered nitrogen-containing heteroaromatic; or

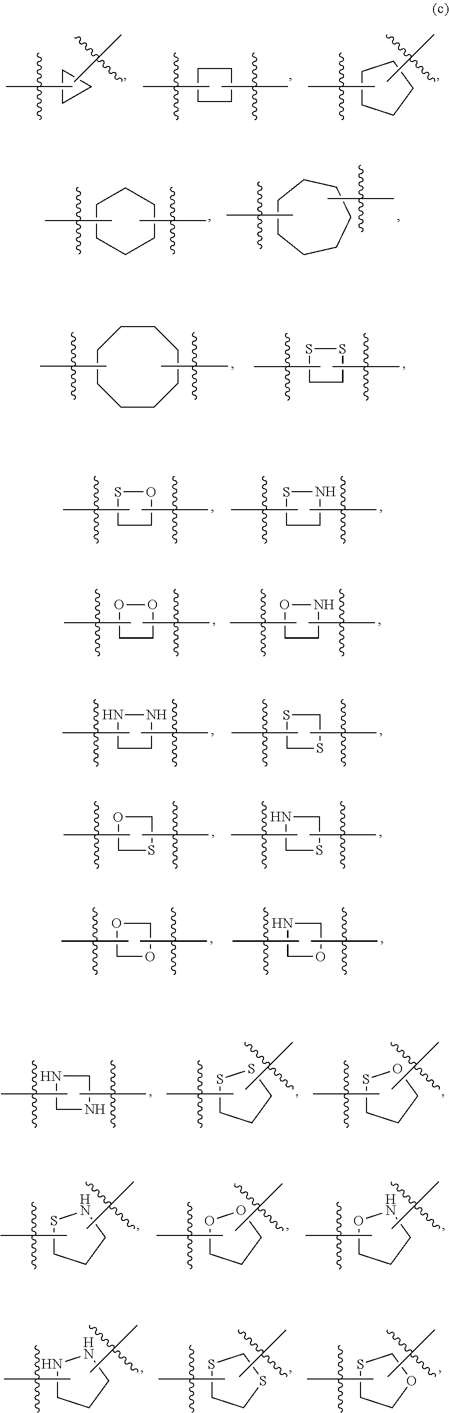

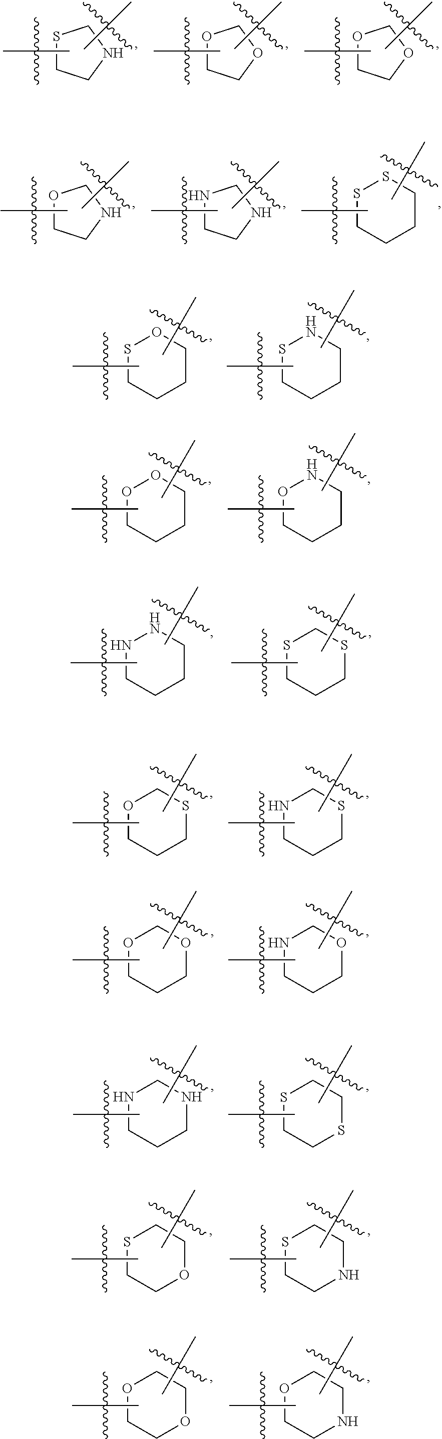

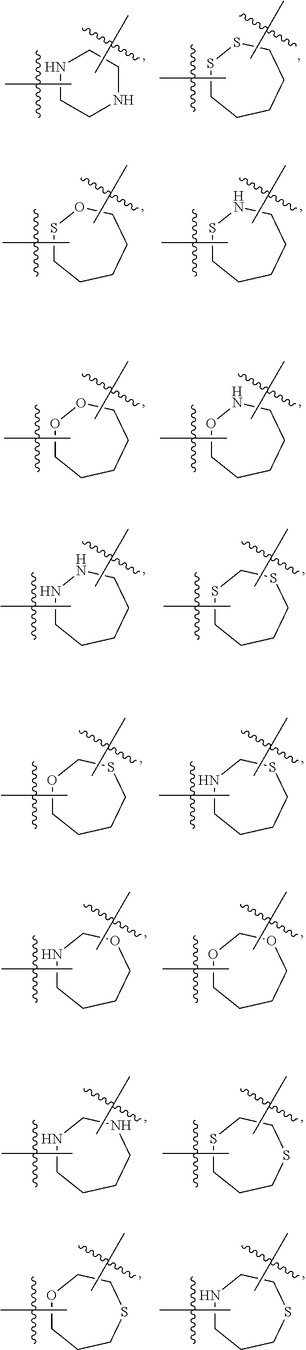

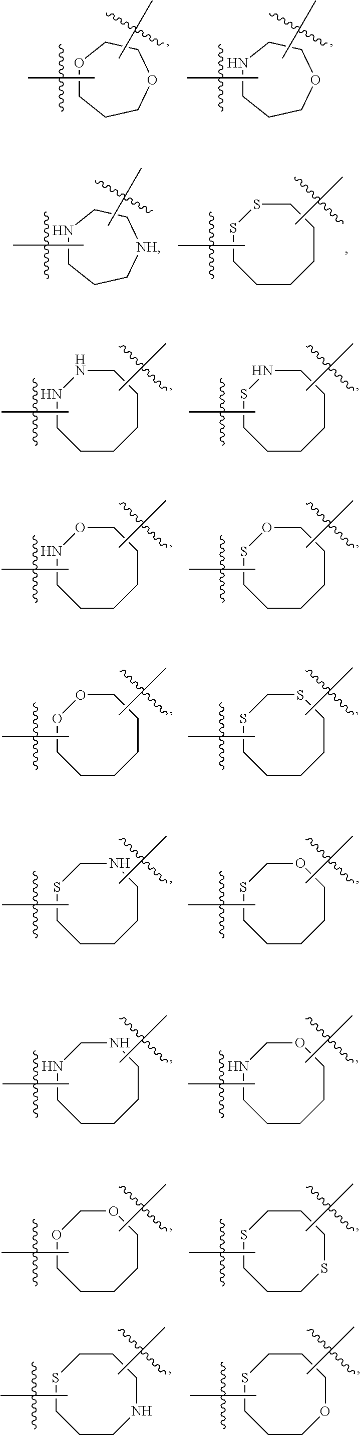

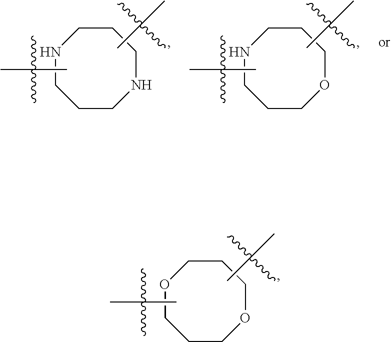

optionally substituted with halo or C1-C6 linear or branched alkyl.

In some variations of formula (34) described in the paragraphs above in which at least one of L1, L2, and L3 is linker (l.2), n is 1-4, 1-3, 1-3, 1-2, 2-4, 2-3, 3-4, 1, 2, 3, or 4.

In some variations of formula (34) described in the paragraphs above, L1 is linker (l.2). In some variations of formula (34) described in the paragraphs above, L2 is linker (l.2). In some variations of formula (34) described in the paragraphs above, L3 is linker (l.2). In some variations of formula (34) described in the paragraphs above, L1 and L2 are linker (l.2). In some variations of formula (34) described in the paragraphs above, L1 and L3 are linker (l.2). In some variations of formula (34) described in the paragraphs above, L2 and L3 are linker (l.2). In some variations of formula (34) described in the paragraphs above, L1, L2, and L3 are linker (l.2).

In some variations of formula (34) described in the paragraphs above, at least one of L1, L2, and L3 is linker (m.1):

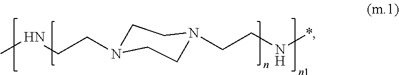

in which n is 0-6, n₁ is 1-4, and * is the attachment site for the cationic dye moiety D.

In some variations of formula (34) in which at least one of L1, L2, and L3 is linker (m.1), n is 0-6, 0-5, 0-4, 0-3, 0-2, 0-1, 1-6, 1-5, 1-4, 1-3, 1-2, 2-6, 2-5, 2-4, 2-3, 3-6, 3-5, 3-4, 4-6, 4-5, 5-6, 0, 1, 2, 3, 4, 5, or 6.

In some variations of formula (34) described in the paragraphs above in which at least one of L1, L2, and L3 is linker (m.1), n₁ is 1-4, 1-3, 1-2, 2-4, 2-3, 3-4, 1, 2, 3, or 4.

In some variations of formula (34) described in the paragraphs above, L1 is linker (m.1). In some variations of formula (34) described in the paragraphs above, L2 is linker (m.1). In some variations of formula (34) described in the paragraphs above, L3 is linker (m.1). In some variations of formula (34) described in the paragraphs above, L1 and L2 are linker (m.1). In some variations of formula (34) described in the paragraphs above, L1 and L3 are linker (m.1). In some variations of formula (34) described in the paragraphs above, L2 and L3 are linker (m.1). In some variations of formula (34) described in the paragraphs above, L1, L2, and L3 are linker (m.1).

In some variations of formula (34) described in the paragraphs above, at least one of L1, L2, and L3 is linker (n.1):

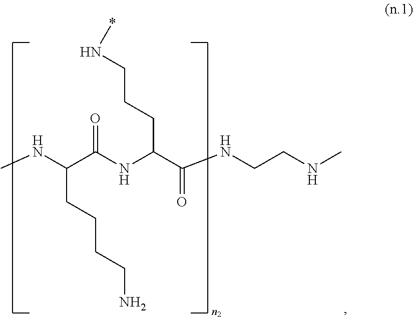

in which n₂ is 1-5 and * is the attachment site for the cationic dye moiety D.

In some variations of formula (34) in which at least one of L1, L2, and L3 is linker (n.1), n₂ is 1-5, 1-4, 1-3, 1-2, 2-5, 2-4, 2-3, 3-5, 3-4, 4-5, 1, 2, 3, 4, or 5.

In some variations of formula (34) described in the paragraphs above, L1 is linker (n.1). In some variations of formula (34) described in the paragraphs above, L2 is linker (n.1). In some variations of formula (34) described in the paragraphs above, L3 is linker (n.1). In some variations of formula (34) described in the paragraphs above, L1 and L2 are linker (n.1). In some variations of formula (34) described in the paragraphs above, L1 and L3 are linker (n.1). In some variations of formula (34) described in the paragraphs above, L2 and L3 are linker (n.1). In some variations of formula (34) described in the paragraphs above, L1, L2, and L3 are linker (n.1).

In some variations of formula (34) described in the paragraphs above, at least one of L1, L2, and L3 is linker (n.2):

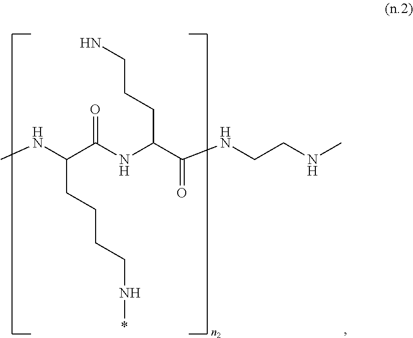

in which n₂ is 1-5 and * is the attachment site for the cationic dye moiety D.

In some variations of formula (34) in which at least one of L1, L2, and L3 is linker (n.2), n₂ is 1-5, 1-4, 1-3, 1-2, 2-5, 2-4, 2-3, 3-5, 3-4, 4-5, 1, 2, 3, 4, or 5.

In some variations of formula (34) described in the paragraphs above, L1 is linker (n.2). In some variations of formula (34) described in the paragraphs above, L2 is linker (n.2). In some variations of formula (34) described in the paragraphs above, L3 is linker (n.2). In some variations of formula (34) described in the paragraphs above, L1 and L2 are linker (n.2). In some variations of formula (34) described in the paragraphs above, L1 and L3 are linker (n.2). In some variations of formula (34) described in the paragraphs above, L2 and L3 are linker (n.2). In some variations of formula (34) described in the paragraphs above, L1, L2, and L3 are linker (n.2).

In some variations of formula (34) described in the paragraphs above, at least one of L1, L2, and L3 is linker (o):

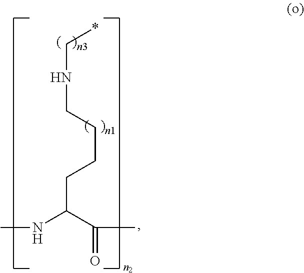

in which in which n₁ is 0-5, n₂ is 1-5, n3 is 0-5, and * is the attachment site for the cationic dye moiety D.

In some variations of formula (34) in which at least one of L1, L2, and L3 is linker (o), n₁ is 0-5, 0-4, 0-3, 0-2, 0-1, 1-5, 1-4, 1-3, 1-2, 2-5, 2-4, 2-3, 3-5, 3-4, 4-5, 0, 1, 2, 3, 4, or 5.

In some variations of formula (34) described in the paragraphs above in which at least one of L1, L2, and L3 is linker (o), n₂ is 1-5, 1-4, 1-3, 1-2, 2-5, 2-4, 2-3, 3-5, 3-4, 4-5, 1, 2, 3, 4, or 5.

In some variations of formula (34) described in the paragraphs above in which at least one of L1, L2, and L3 is linker (o), n₃ is 0-5, 0-4, 0-3, 0-2, 0-1, 1-5, 1-4, 1-3, 1-2, 2-5, 2-4, 2-3, 3-5, 3-4, 4-5, 0, 1, 2, 3, 4, or 5.

In some variations of formula (34) described in the paragraphs above, L1 is linker (o). In some variations of formula (34) described in the paragraphs above, L2 is linker (o). In some variations of formula (34) described in the paragraphs above, L3 is linker (o). In some variations of formula (34) described in the paragraphs above, L1 and L2 are linker (o). In some variations of formula (34) described in the paragraphs above, L1 and L3 are linker (o). In some variations of formula (34) described in the paragraphs above, L2 and L3 are linker (o). In some variations of formula (34) described in the paragraphs above, L1, L2, and L3 are linker (o).

In some variations of formula (34) described in the paragraphs above, at least one of L1, L2, and L3 is linker (p):

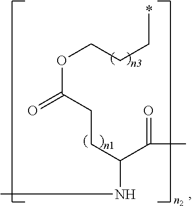

in which n₁ is 0-5, n₂ is 1-5, n3 is 0-5, and * is the attachment site for the cationic dye moiety D.

In some variations of formula (34) described above in which at least one of L1, L2, L3, and L4 is linker (p), n₁ is 0-5, 0-4, 0-3, 0-2, 0-1, 1-5, 1-4, 1-3, 1-2, 2-5, 2-4, 2-3, 3-5, 3-4, 4-5, 0, 1, 2, 3, 4, or 5.

In some variations of formula (34) described above in which at least one of L1, L2, L3, and L4 is linker (p), n₂ is 1-5, 1-4, 1-3, 1-2, 2-5, 2-4, 2-3, 3-5, 3-4, 4-5, 1, 2, 3, 4, or 5.

In some variations of formula (34) described above in which at least one of L1, L2, L3, and L4 is linker (p), n₃ is 0-5, 0-4, 0-3, 0-2, 0-1, 1-5, 1-4, 1-3, 1-2, 2-5, 2-4, 2-3, 3-5, 3-4, 4-5, 0, 1, 2, 3, 4, or 5.

In some variations of formula (34) described in the paragraphs above, L1 is linker (p). In some variations of formula (34) described in the paragraphs above, L2 is linker (p). In some variations of formula (34) described in the paragraphs above, L3 is linker (p). In some variations of formula (34) described in the paragraphs above, L1 and L2 are linker (p). In some variations of formula (34) described in the paragraphs above, L1 and L3 are linker (p). In some variations of formula (34) described in the paragraphs above, L2 and L3 are linker (p). In some variations of formula (34) described in the paragraphs above, L1, L2, and L3 are linker (p).

In some variations of formula (34) described in the paragraphs above, at least one of L1, L2, and L3 is linker (q):

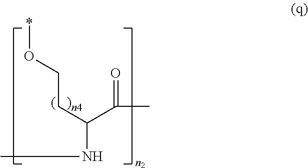

in which n₄ is 0-5, n₂ is 1-5, and * is the attachment site for the cationic dye moiety D.

In some variations of formula (34) in which at least one of L1, L2, and L3 is linker (o), n₄ is 0-5, 0-4, 0-3, 0-2, 0-1, 1-5, 1-4, 1-3, 1-2, 2-5, 2-4, 2-3, 3-5, 3-4, 4-5, 0, 1, 2, 3, 4, or 5.

In some variations of formula (34) described above in which at least one of L1, L2, L3, and L4 is linker (q), n₂ is 1-5, 1-4, 1-3, 1-2, 2-5, 2-4, 2-3, 3-5, 3-4, 4-5, 1, 2, 3, 4, or 5.

In some variations of formula (34) described in the paragraphs above, L1 is linker (q). In some variations of formula (34) described in the paragraphs above, L2 is linker (q). In some variations of formula (34) described in the paragraphs above, L3 is linker (q). In some variations of formula (34) described in the paragraphs above, L1 and L2 are linker (q). In some variations of formula (34) described in the paragraphs above, L1 and L3 are linker (q). In some variations of formula (34) described in the paragraphs above, L2 and L3 are linker (q). In some variations of formula (34) described in the paragraphs above, L1, L2, and L3 are linker (q).

In some variations of formula (34) described in the paragraphs above, at least one of L1, L2, and L3 is linker (r):

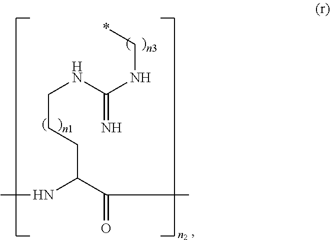

in which n₁ is 0-5, n₂ is 1-5, n3 is 0-5, and * is the attachment site for the cationic dye moiety D.

In some of variations of formula (34) in which at least one of L1, L2, and L3 is linker (r), n₁ is 0-5, 0-4, 0-3, 0-2, 0-1, 1-5, 1-4, 1-3, 1-2, 2-5, 2-4, 2-3, 3-5, 3-4, 4-5, 0, 1, 2, 3, 4, or 5.

In some of variations of formula (34) described in the paragraphs above in which at least one of L1, L2, and L3 is linker (r), n₂ is 1-5, 1-4, 1-3, 1-2, 2-5, 2-4, 2-3, 3-5, 3-4, 4-5, 1, 2, 3, 4, or 5.

In some of variations of formula (34) in which at least one of L1, L2, and L3 is linker (r), n3 is 0-5, 0-4, 0-3, 0-2, 0-1, 1-5, 1-4, 1-3, 1-2, 2-5, 2-4, 2-3, 3-5, 3-4, 4-5, 0, 1, 2, 3, 4, or 5.

In some variations of formula (34) described in the paragraphs above, L1 is linker (r). In some variations of formula (34) described in the paragraphs above, L2 is linker (r). In some variations of formula (34) described in the paragraphs above, L3 is linker (r). In some variations of formula (34) described in the paragraphs above, L1 and L2 are linker (r). In some variations of formula (34) described in the paragraphs above, L1 and L3 are linker (r). In some variations of formula (34) described in the paragraphs above, L2 and L3 are linker (r). In some variations of formula (34) described in the paragraphs above, L1, L2, and L3 are linker (r).

In some variations of formula (34) described in the paragraphs above, at least one of L1, L2, and L3 is linker (s):

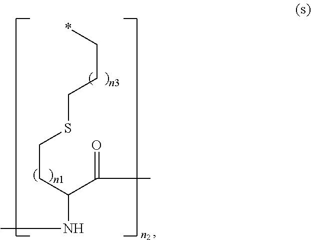

in which n₁ is 0-5, n₂ is 1-5, n₃ is 0-5, and * is the attachment site for the cationic dye moiety D.

In some variations of formula (34) described in the paragraphs above, L1 is linker (s). In some variations of formula (34) described in the paragraphs above, L2 is linker (s). In some variations of formula (34) described in the paragraphs above, L3 is linker (s). In some variations of formula (34) described in the paragraphs above, L1 and L2 are linker (s). In some variations of formula (34) described in the paragraphs above, L1 and L3 are linker (s). In some variations of formula (34) described in the paragraphs above, L2 and L3 are linker (s). In some variations of formula (34) described in the paragraphs above, L1, L2, and L3 are linker (s).

In some variations of formula (34) described in the paragraphs above, L1 is absent. In some variations of formula (34) described in the paragraphs above, L2 is absent. In some variations of formula (34) described in the paragraphs above, L3 is absent. In some variations of formula (34) described in the paragraphs above, L1 and L2 are absent. In some variations of formula (34) described in the paragraphs above, L1 and L3 are absent. In some variations of formula (34) described in the paragraphs above, L2 and L3 are absent. In some variations of formula (34) described in the paragraphs above, L1, L2, and L3 are absent.

In some variations of formula (34), each of L1, L2, and L3 is a different linker. In some variations of formula (34), each of L1, L2, and L3 is the same linker. In some variations of formula (34), L1 is a first linker and each of L2 and L3 is the same second linker, wherein the first and second linkers are different. In some variations of formula (34), L2 is a first linker, and each of L1 and L3 is the same second linker, wherein the first and second linkers are different. In some variations of formula (34), L3 is a first linker and each of L1 and L2 is the same second linker, wherein the first and second linkers are different.

In some variations of formula (34) described in the paragraphs above, D1 is safranin-O. In some variations of formula (34) described in the paragraphs above, D2 is safranin-O. In some variations of formula (34) described in the paragraphs above, D3 is safranin-O. In some variations of formula (34) described in the paragraphs above, D1 and D2 are safranin-O. In some variations of formula (34) described in the paragraphs above, D1 and D3 are safranin-O. In some variations of formula (34) described in the paragraphs above, D2 and D4 are safranin-O.

In some variations of formula (34) described in the paragraph above, the pendant phenyl ring of D1 is unsubstituted. In some variations of formula (34) described in the paragraph above, the pendant phenyl ring of D1 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D1 is substituted, the substituents are selected independently from NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D1 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (34) described in the two paragraphs above, the pendant phenyl ring of D2 is unsubstituted. In some variations of formula (34) described in the paragraph above, the pendant phenyl ring of D2 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D2 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D2 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

In some variations of formula (34) described in the three paragraphs above, the pendant phenyl ring of D3 is unsubstituted. In some variations of formula (34) described in the paragraph above, the pendant phenyl ring of D3 is substituted with 1-3 (e.g., 1-3, 1-2, 1, 2, or 3) electron-donating or electron-withdrawing groups, which may be at any available position on the pendant phenyl ring. In some embodiments in which the pendant phenyl ring of D3 is substituted, the substituents are selected independently from —NH₂, —NHR, —NR₂, —OH, —O⁻, —NHCOCH₃, —NHCOR, —OCH₃, —OR, —C₂H₅, —R, and —C₆H₅, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl). In some embodiments in which the pendant phenyl ring of D3 is substituted, the substituents are selected independently from —NO₂, —NR₃⁺, halo (e.g., F, Br, Cl, I), trihalide (e.g., —CF₃, —CCl₃, —CBr₃, —CI₃), —CN, —SO₃H, —COOH, —COOR, —CHO, and —COR, wherein R is C1-C6 linear or branched alkyl (e.g., C1-C6, C1-C5, C1-C4, C1-C3, C1-C2, C1, C2-C6, C2-C5, C2-C4, C2-C3, C2, C3-C6, C3-C5, C3-C4, C3, C4-C6, C4-C5, C4, C5-C6, C5, or C6 linear or branched alkyl).

Examples of cationic dye dimers and trimers comprising safranin-O are provided below in Table 1. One of skill in the art can readily visualize and prepare other cationic dye multimers in which other cationic dye moieties are used in place of one or more of the safranin-O moieties.

TABLE 1

| Cmpd | |
|---|---|
| No. | Structure |
| 1 | 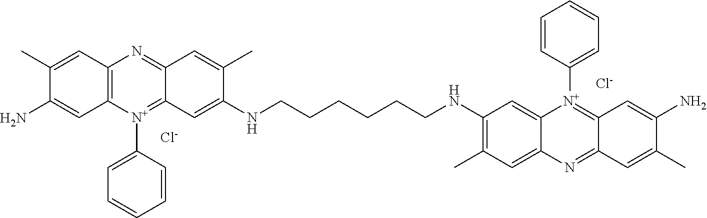 [SMILES: Cc1cc2nc3cc(C)c(NCCCCCCNc4cc5c(cc4C)nc4cc(C)c(N)cc4[n+]5-c4ccccc4)cc3[n+](-c3ccccc3)c2cc1N ; [Cl-] ; [Cl-]] |
| 2 | 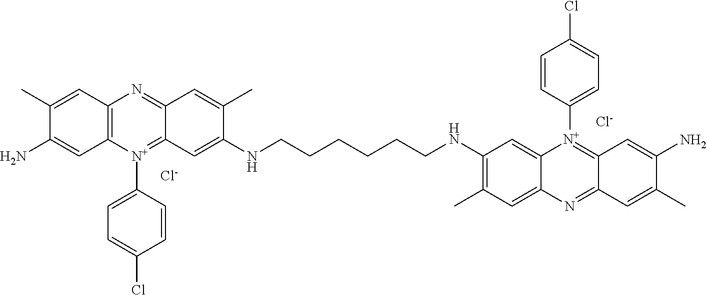 [SMILES: Cc1cc2nc3cc(C)c(NCCCCCCNc4cc5c(cc4C)nc4cc(C)c(N)cc4[n+]5-c4ccc(Cl)cc4)cc3[n+](-c3ccc(Cl)cc3)c2cc1N ; [Cl-] ; [Cl-]] |
| 3 | 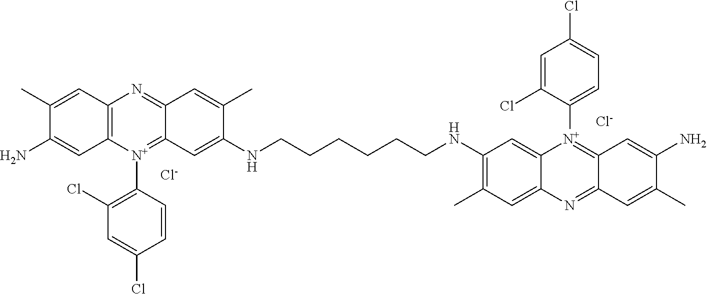 [SMILES: Cc1cc2nc3cc(C)c(NCCCCCCNc4cc5c(cc4C)nc4cc(C)c(N)cc4[n+]5-c4ccc(Cl)cc4Cl)cc3[n+](-c3ccc(Cl)cc3Cl)c2cc1N ; [Cl-] ; [Cl-]] |
| 4 | 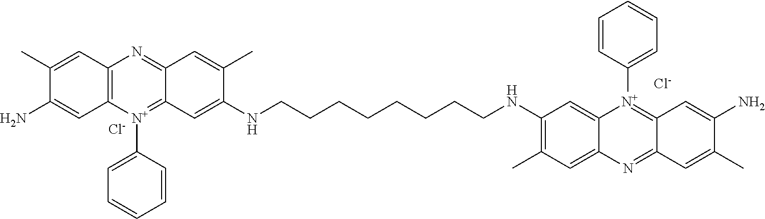 [SMILES: Cc1cc2nc3cc(C)c(NCCCCCCCCNc4cc5c(cc4C)nc4cc(C)c(N)cc4[n+]5-c4ccccc4)cc3[n+](-c3ccccc3)c2cc1N ; [Cl-] ; [Cl-]] |
| 5 | 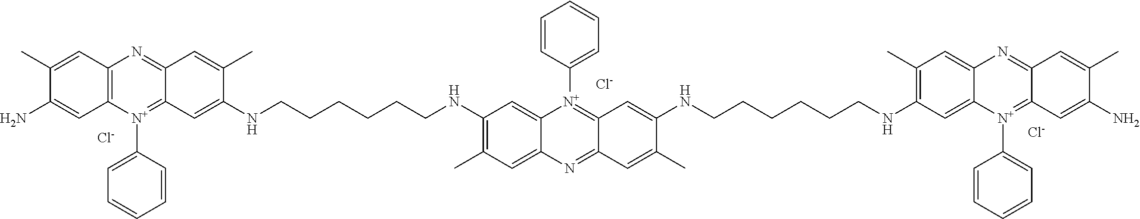 [SMILES: Cc1cc2nc3cc(C)c(NCCCCCCNc4cc5c(cc4C)nc4cc(C)c(NCCCCCCNc6cc7c(cc6C)nc6cc(C)c(N)cc6[n+]7-c6ccccc6)cc4[n+]5-c4ccccc4)cc3[n+](-c3ccccc3)c2cc1N ; [Cl-] ; [Cl-] ; [Cl-]] |
| 6 | 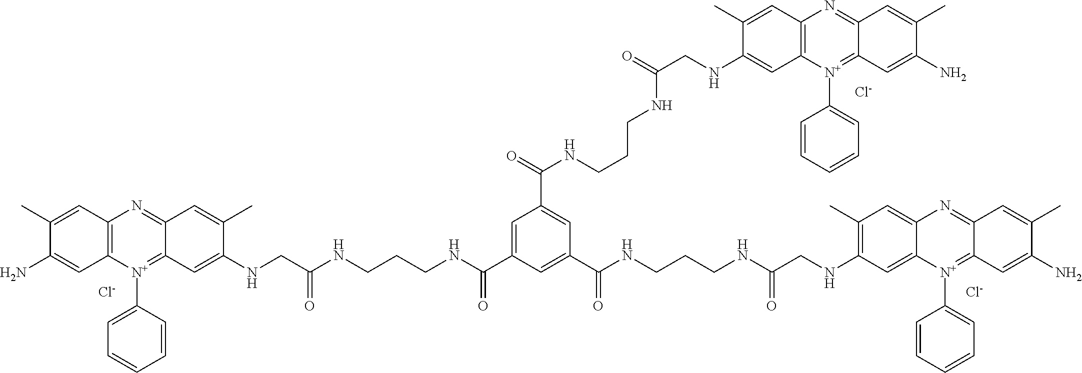 [SMILES: Cc1cc2nc3cc(C)c(NCC(=O)NCCCNC(=O)c4cc(C(=O)NCCCNC(=O)CNc5cc6c(cc5C)nc5cc(C)c(N)cc5[n+]6-c5ccccc5)cc(C(=O)NCCCNC(=O)CNc5cc6c(cc5C)nc5cc(C)c(N)cc5[n+]6-c5ccccc5)c4)cc3[n+](-c3ccccc3)c2cc1N ; [Cl-] ; [Cl-] ; [Cl-]] |
| 7 | 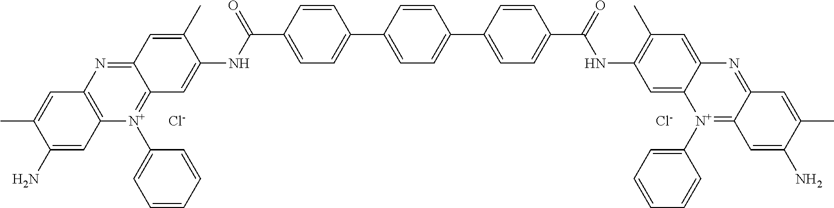 |
| 8 | 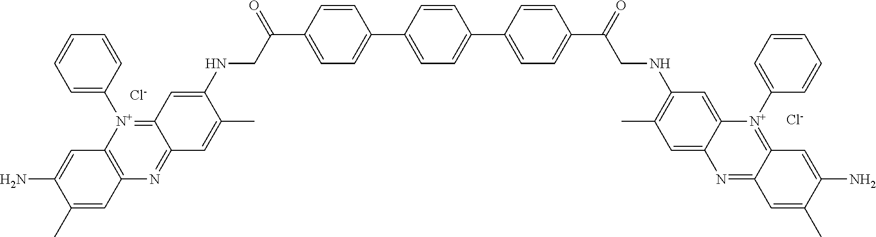 [SMILES: Cc1cc2nc3cc(C)c(NCC(=O)c4ccc(-c5ccc(-c6ccc(C(=O)CNc7cc8c(cc7C)nc7cc(C)c(N)cc7[n+]8-c7ccccc7)cc6)cc5)cc4)cc3[n+](-c3ccccc3)c2cc1N ; [Cl-] ; [Cl-]] |
| 9 | 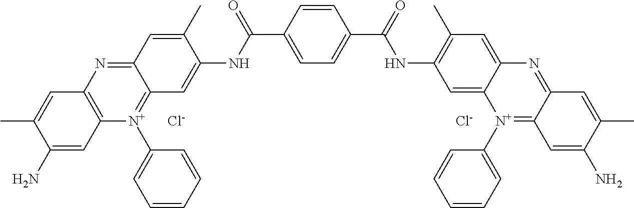 |
| 10 | 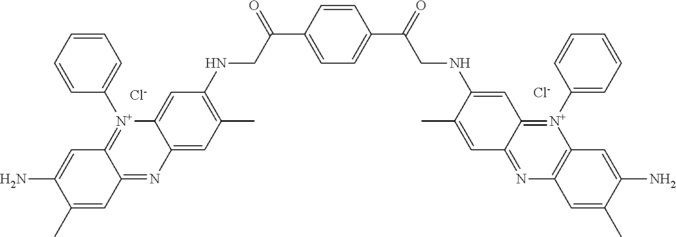 [SMILES: Cc1cc2nc3cc(C)c(NCC(=O)c4ccc(C(=O)CNc5cc6c(cc5C)nc5cc(C)c(N)cc5[n+]6-c5ccccc5)cc4)cc3[n+](-c3ccccc3)c2cc1N ; [Cl-] ; [Cl-]] |
| 11 | 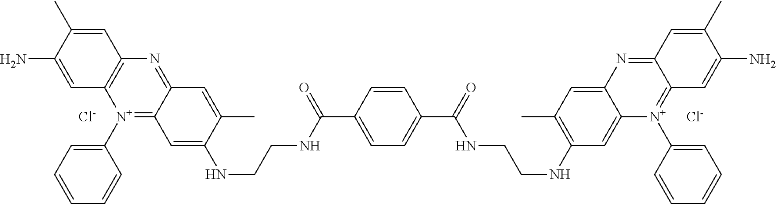 [SMILES: Cc1cc2nc3cc(C)c(NCCNC(=O)c4ccc(C(=O)NCCNc5cc6c(cc5C)nc5cc(C)c(N)cc5[n+]6-c5ccccc5)cc4)cc3[n+](-c3ccccc3)c2cc1N ; [Cl-] ; [Cl-]] |
| 12 | 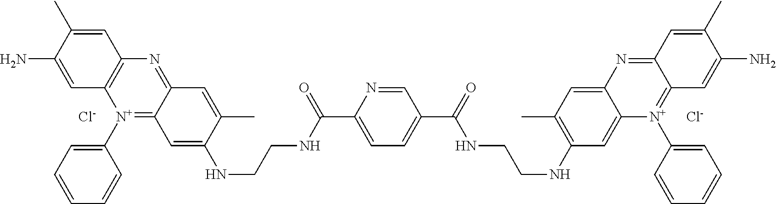 [SMILES: Cc1cc2nc3cc(C)c(NCCNC(=O)c4ccc(C(=O)NCCNc5cc6c(cc5C)nc5cc(C)c(N)cc5[n+]6-c5ccccc5)nc4)cc3[n+](-c3ccccc3)c2cc1N ; [Cl-] ; [Cl-]] |
| 13 | 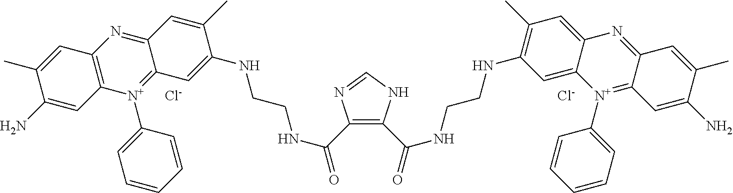 |
| 14 | 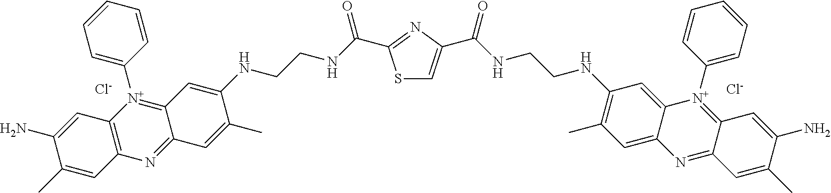 [SMILES: Cc1cc2nc3cc(C)c(NCCNC(=O)c4csc(C(=O)NCCNc5cc6c(cc5C)nc5cc(C)c(N)cc5[n+]6-c5ccccc5)n4)cc3[n+](-c3ccccc3)c2cc1N ; [Cl-] ; [Cl-]] |
| 15 | 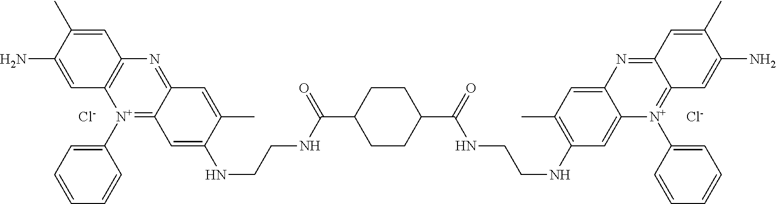 [SMILES: Cc1cc2nc3cc(C)c(NCCNC(=O)C4CCC(C(=O)NCCNc5cc6c(cc5C)nc5cc(C)c(N)cc5[n+]6-c5ccccc5)CC4)cc3[n+](-c3ccccc3)c2cc1N ; [Cl-] ; [Cl-]] |
| 16 | 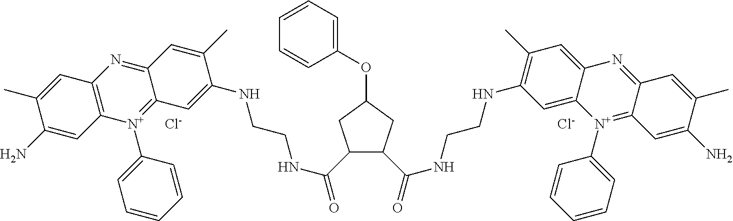 |
| 17 | 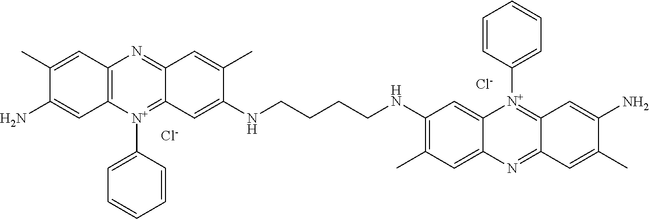 [SMILES: Cc1cc2nc3cc(C)c(NCCCCNc4cc5c(cc4C)nc4cc(C)c(N)cc4[n+]5-c4ccccc4)cc3[n+](-c3ccccc3)c2cc1N ; [Cl-] ; [Cl-]] |
| 18 | 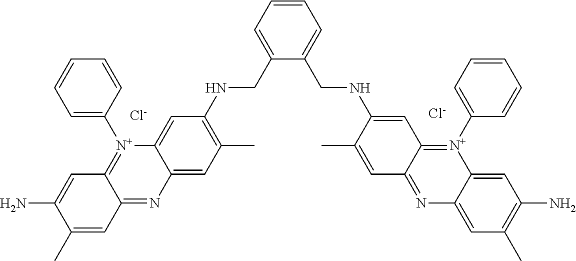 [SMILES: Cc1cc2nc3cc(C)c(NCc4ccccc4CNc4cc5c(cc4C)nc4cc(C)c(N)cc4[n+]5-c4ccccc4)cc3[n+](-c3ccccc3)c2cc1N ; [Cl-] ; [Cl-]] |
| 19 | 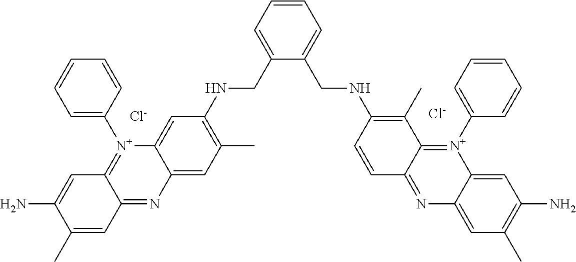 [SMILES: Cc1cc2nc3cc(C)c(NCc4ccccc4CNc4ccc5nc6cc(C)c(N)cc6[n+](-c6ccccc6)c5c4C)cc3[n+](-c3ccccc3)c2cc1N ; [Cl-] ; [Cl-]] |
| 20 | 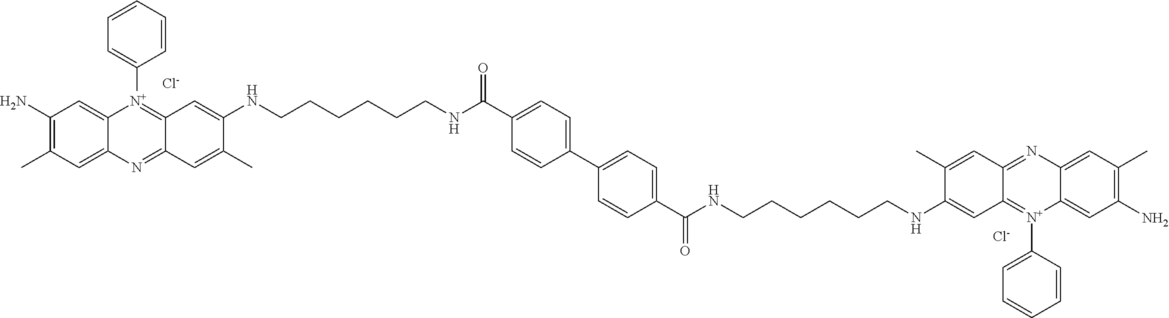 [SMILES: Cc1cc2nc3cc(C)c(NCCCCCCNC(=O)c4ccc(-c5ccc(C(=O)NCCCCCCNc6cc7c(cc6C)nc6cc(C)c(N)cc6[n+]7-c6ccccc6)cc5)cc4)cc3[n+](-c3ccccc3)c2cc1N ; [Cl-] ; [Cl-]] |
| 21 | 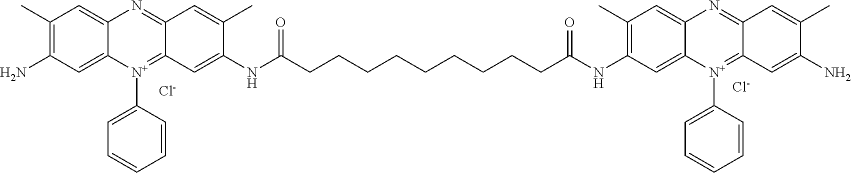 [SMILES: Cc1cc2nc3cc(C)c(NC(=O)CCCCCCCCCC(=O)Nc4cc5c(cc4C)nc4cc(C)c(N)cc4[n+]5-c4ccccc4)cc3[n+](-c3ccccc3)c2cc1N ; [Cl-] ; [Cl-]] |
| 22 | 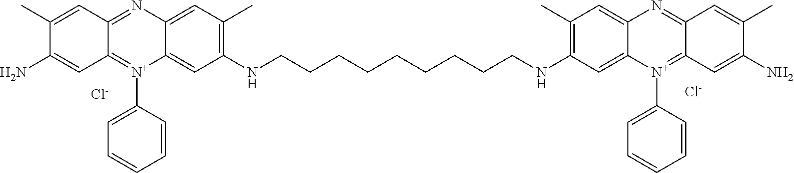 [SMILES: Cc1cc2nc3cc(C)c(NCCCCCCCCCNc4cc5c(cc4C)nc4cc(C)c(N)cc4[n+]5-c4ccccc4)cc3[n+](-c3ccccc3)c2cc1N ; [Cl-] ; [Cl-]] |
| 23 | 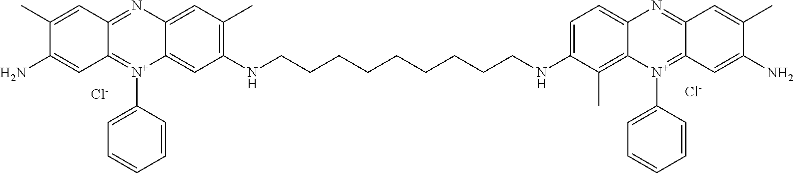 [SMILES: Cc1cc2nc3cc(C)c(NCCCCCCCCCNc4ccc5nc6cc(C)c(N)cc6[n+](-c6ccccc6)c5c4C)cc3[n+](-c3ccccc3)c2cc1N ; [Cl-] ; [Cl-]] |
| 24 | 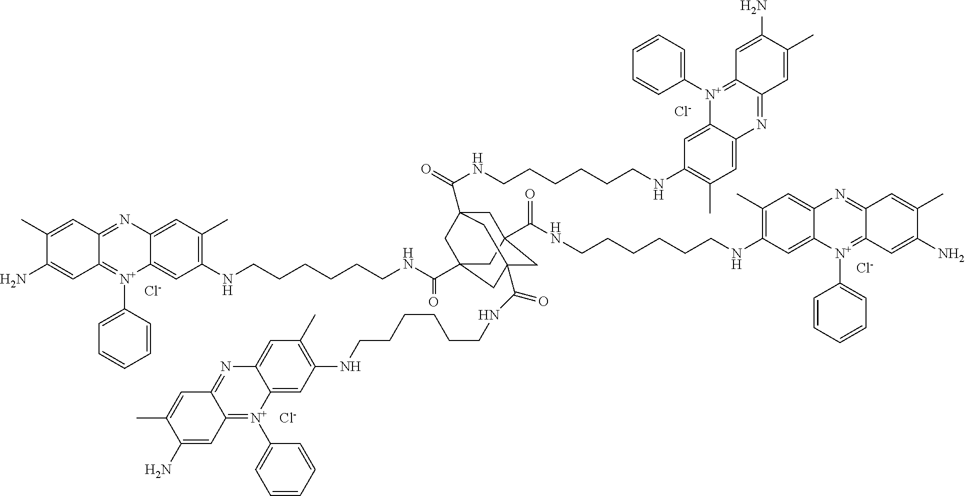 [SMILES: Cc1cc2nc3cc(C)c(NCCCCCCNC(=O)C45CC6(C(=O)NCCCCCCNc7cc8c(cc7C)nc7cc(C)c(N)cc7[n+]8-c7ccccc7)CC(C(=O)NCCCCCCNc7cc8c(cc7C)nc7cc(C)c(N)cc7[n+]8-c7ccccc7)(C4)CC(C(=O)NCCCCCCNc4cc7c(cc4C)nc4cc(C)c(N)cc4[n+]7-c4ccccc4)(C5)C6)cc3[n+](-c3ccccc3)c2cc1N ; [Cl-] ; [Cl-] ; [Cl-] ; [Cl-]] |
| 25 | 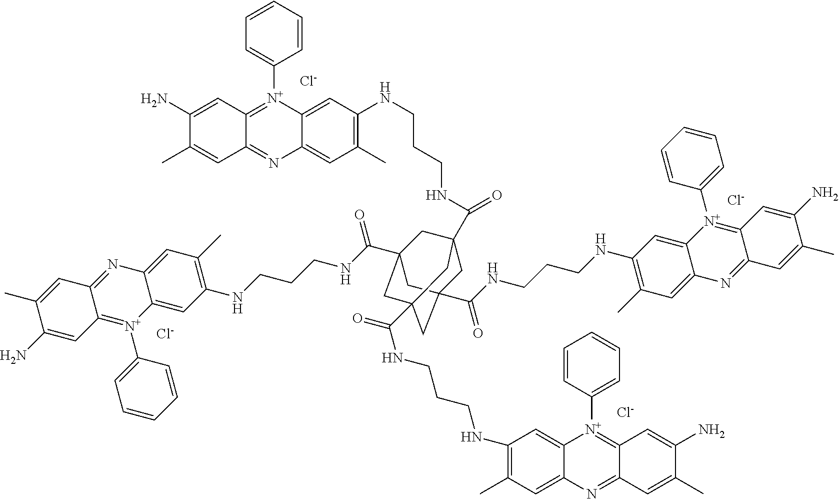 |
| 26 | 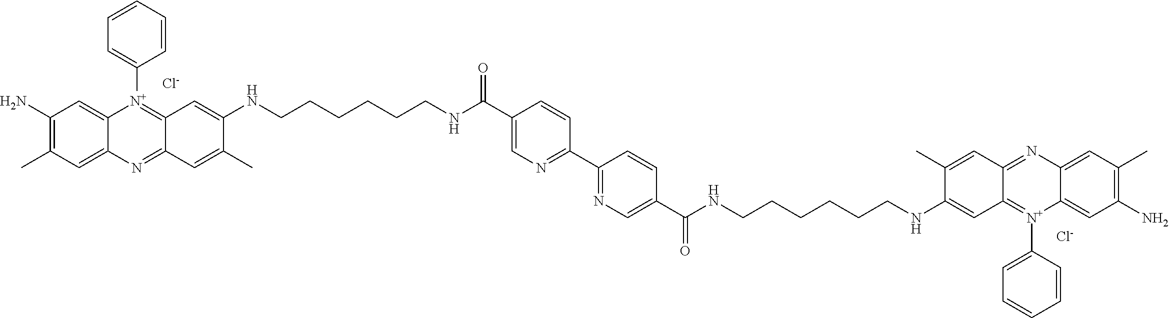 [SMILES: Cc1cc2nc3cc(C)c(NCCCCCCNC(=O)c4ccc(-c5ccc(C(=O)NCCCCCCNc6cc7c(cc6C)nc6cc(C)c(N)cc6[n+]7-c6ccccc6)cn5)nc4)cc3[n+](-c3ccccc3)c2cc1N ; [Cl-] ; [Cl-]] |
| 27 | 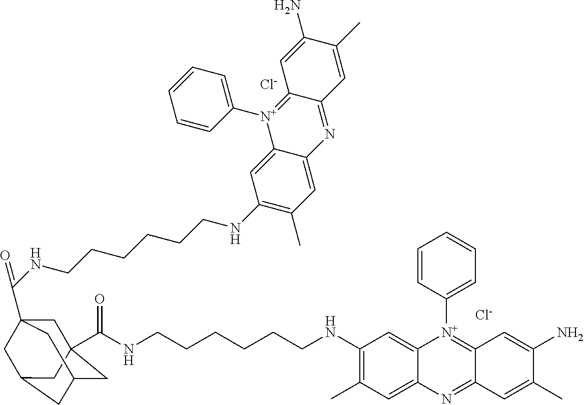 [SMILES: Cc1cc2nc3cc(C)c(NCCCCCCNC(=O)C45CC6CC(C4)CC(C(=O)NCCCCCCNc4cc7c(cc4C)nc4cc(C)c(N)cc4[n+]7-c4ccccc4)(C6)C5)cc3[n+](-c3ccccc3)c2cc1N ; [Cl-] ; [Cl-]] |
| 28 | 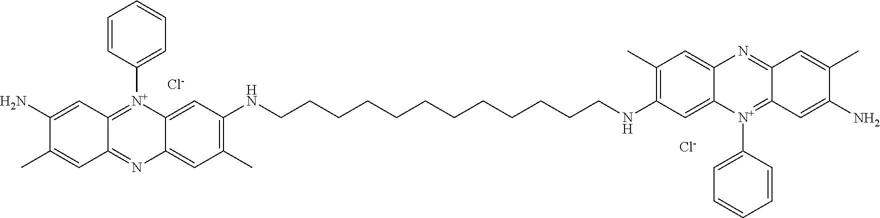 [SMILES: Cc1cc2nc3cc(C)c(NCCCCCCCCCCCCNc4cc5c(cc4C)nc4cc(C)c(N)cc4[n+]5-c4ccccc4)cc3[n+](-c3ccccc3)c2cc1N ; [Cl-] ; [Cl-]] |
| 29 | 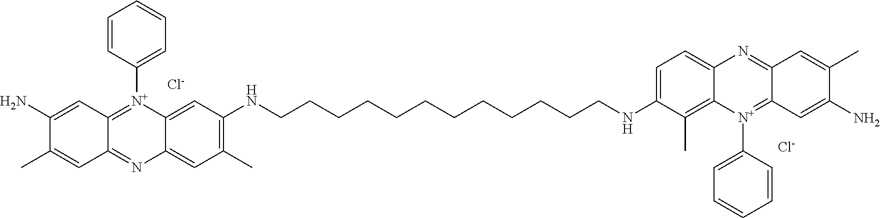 [SMILES: Cc1cc2nc3cc(C)c(NCCCCCCCCCCCCNc4ccc5nc6cc(C)c(N)cc6[n+](-c6ccccc6)c5c4C)cc3[n+](-c3ccccc3)c2cc1N ; [Cl-] ; [Cl-]] |
| 30 | 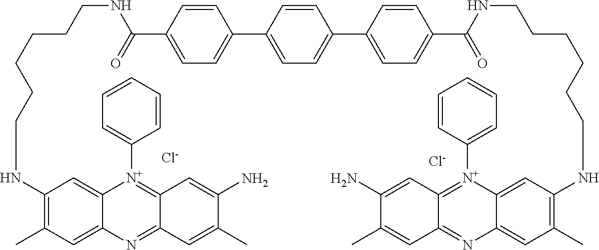 [SMILES: Cc1cc2nc3cc(C)c(NCCCCCCNC(=O)c4ccc(-c5ccc(-c6ccc(C(=O)NCCCCCCNc7cc8c(cc7C)nc7cc(C)c(N)cc7[n+]8-c7ccccc7)cc6)cc5)cc4)cc3[n+](-c3ccccc3)c2cc1N ; [Cl-] ; [Cl-]] |
| 31 | 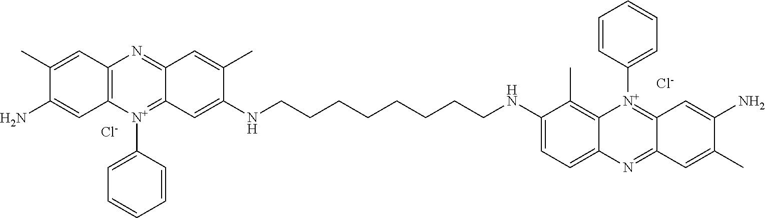 [SMILES: Cc1cc2nc3cc(C)c(NCCCCCCCCNc4ccc5nc6cc(C)c(N)cc6[n+](-c6ccccc6)c5c4C)cc3[n+](-c3ccccc3)c2cc1N ; [Cl-] ; [Cl-]] |
| 32 | 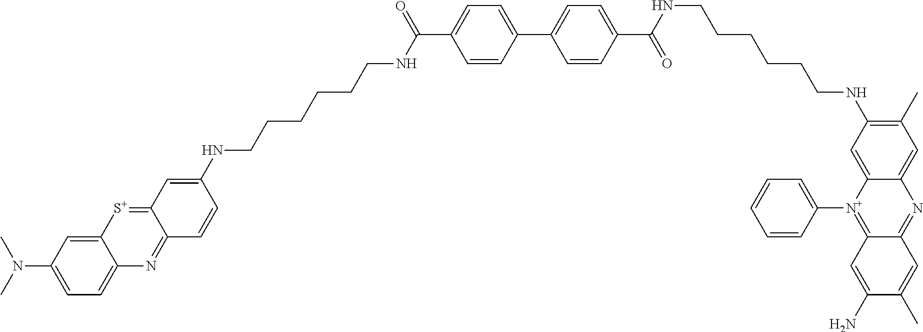 |
| 33 | 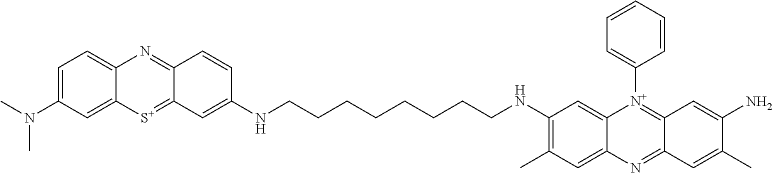 [SMILES: Cc1cc2nc3cc(C)c(NCCCCCCCCNc4ccc5nc6ccc(N(C)C)cc6[s+]c5c4)cc3[n+](-c3ccccc3)c2cc1N] |

Each reference cited in this disclosure is incorporated herein in its entirety. The following examples illustrate but do not limit the scope of the disclosure set forth above.

### Example 1. Schematic Synthesis Plan for Preparing a Cationic Dye Dimer

This example provides a synthesis plan for assembling a cationic dye dimer using safranin-O.

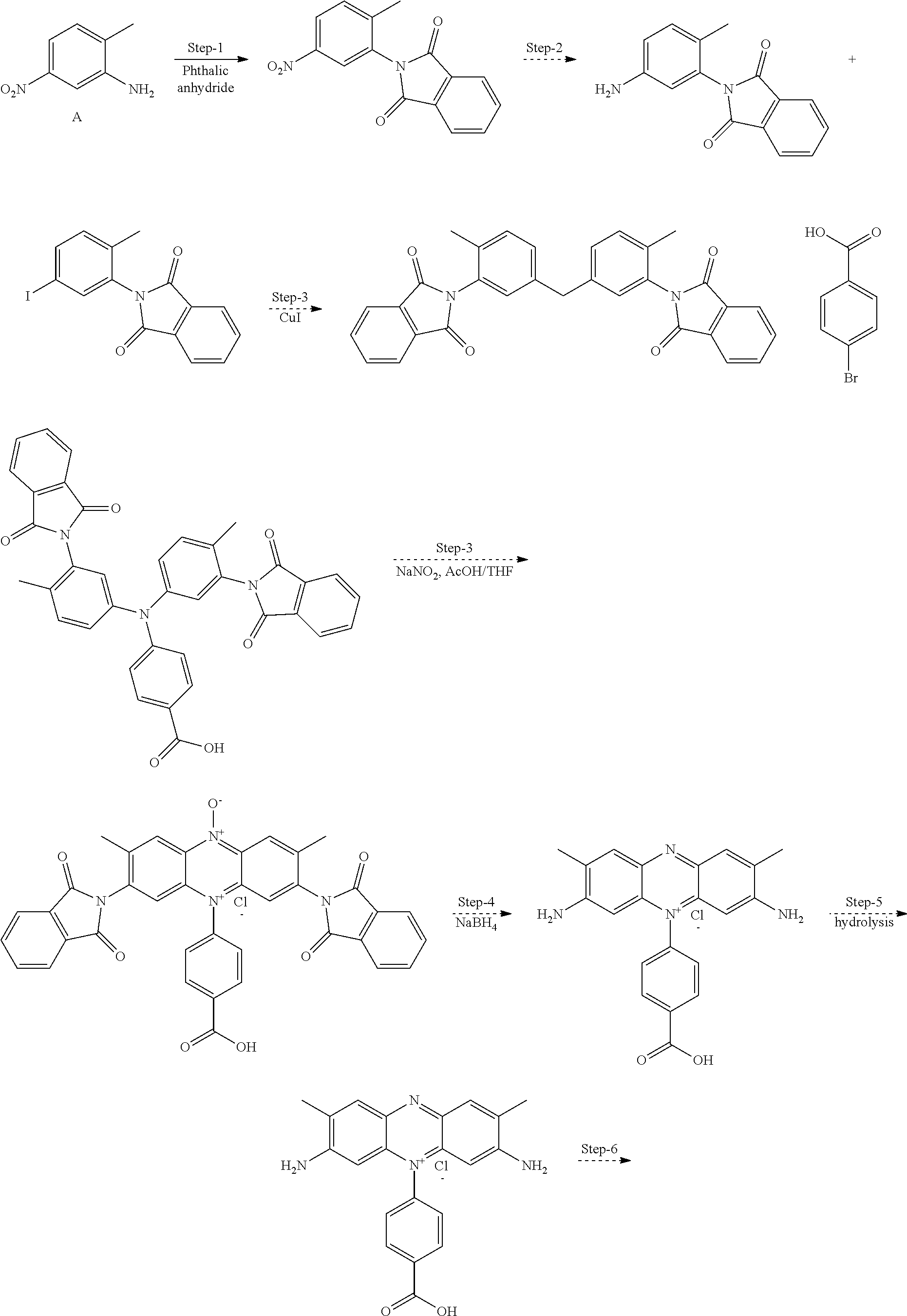

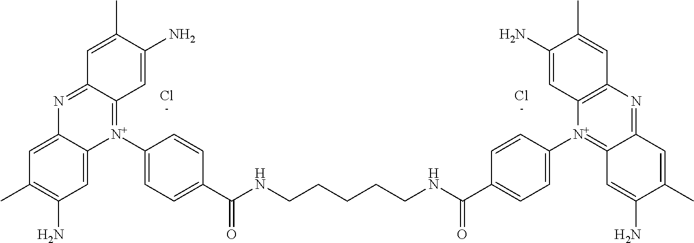

### Example 2. Methods for Preparing Safranin-O Derivatives

This example provides schematic synthesis plans for safranin-O derivatives.

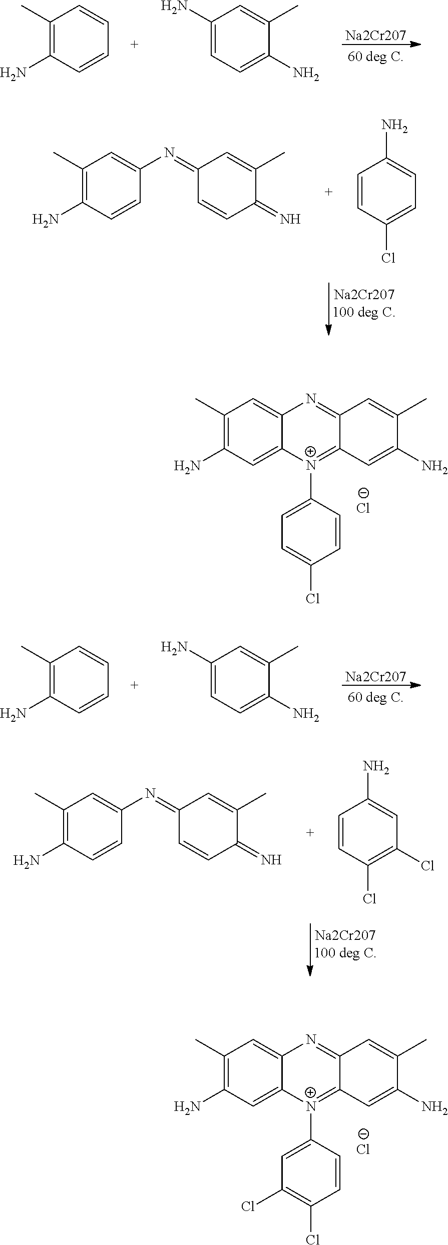

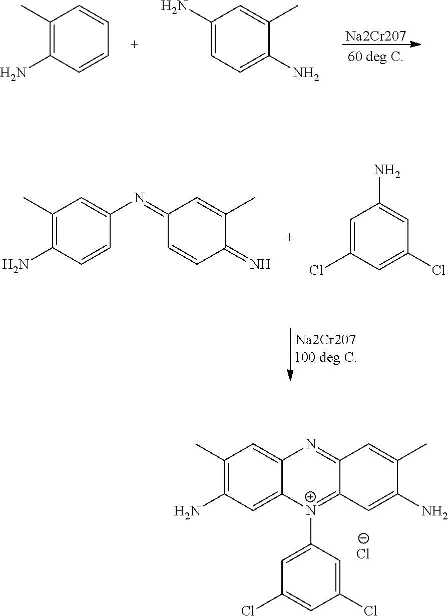

### Example 3. Examples of Linking Cationic Dye Moieties Using Diamino Linkers

Cationic dye moieties can be linked using acetamido groups through diamino linkers, as illustrated below:

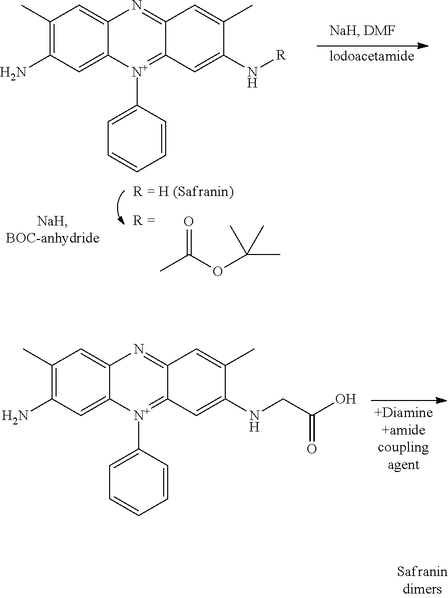

Linkers which can be used include linkers incorporating alkyl chains, such as

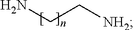

linkers incorporating ethylene glycol moieties, such as

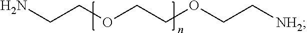

linkers containing amines which can be positively charged, such as

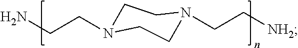

and linkers incorporating alkyl chains which contain positively charged amino acids such as Lys (illustrated below), His, or Arg:

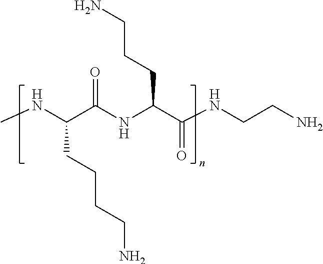

(in this instance, the ε-amino group would initially be in a protected state).

### Example 4. Preparation of Linear Cationic Dye Multimers

Cationic dye moieties can be linked linearly using the scheme below, which illustrates preparation of a linear trimer comprising safranin-O moieties. The same process can be repeated as desired to prepare a cationic dye multimer comprising e.g., 4, 5, or 6, cationic dye moieties.

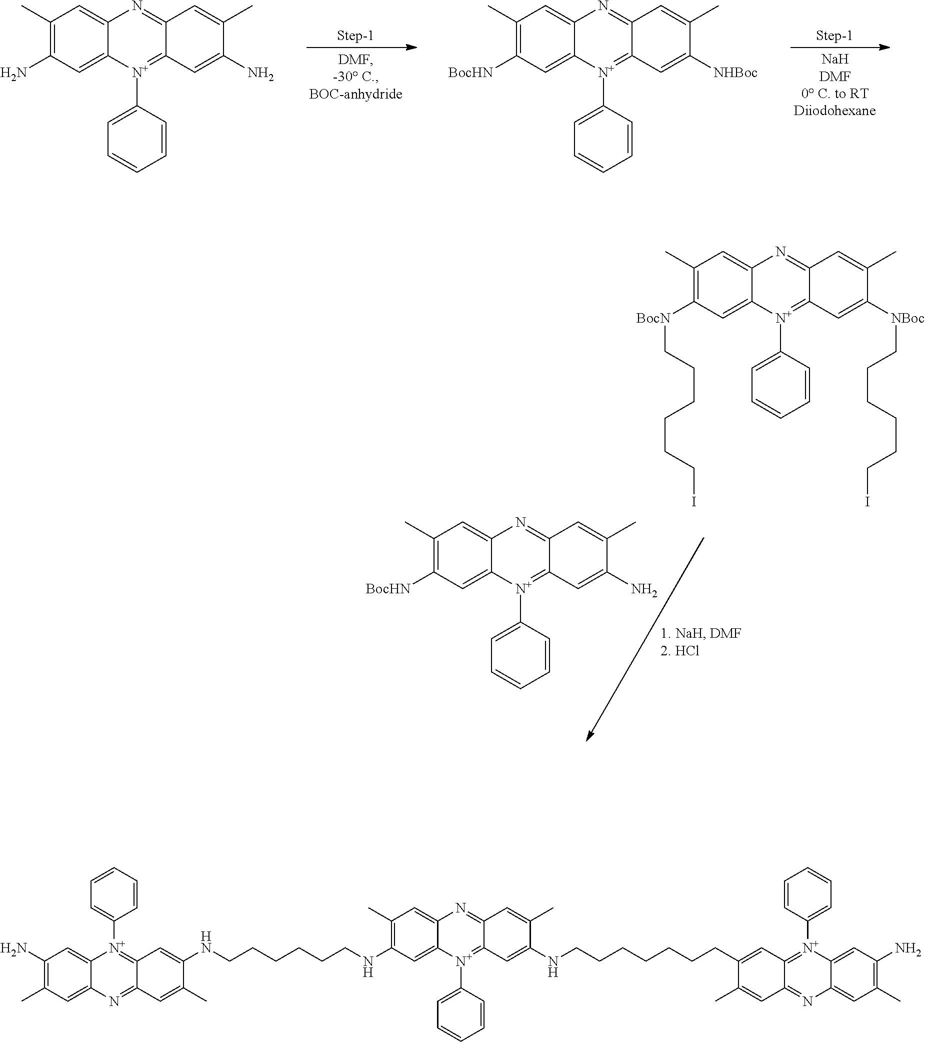 [SMILES: Cc1cc2nc3cc(C)c(N)cc3[n+](-c3ccccc3)c2cc1N ; Cc1cc2nc3cc(C)c(NC(=O)OC(C)(C)C)cc3[n+](-c3ccccc3)c2cc1NC(=O)OC(C)(C)C ; Cc1cc2nc3cc(C)c(N(CCCCCCI)C(=O)OC(C)(C)C)cc3[n+](-c3ccccc3)c2cc1N(CCCCCCI)C(=O)OC(C)(C)C ; Cc1cc2nc3cc(C)c(NC(=O)OC(C)(C)C)cc3[n+](-c3ccccc3)c2cc1N ; Cc1cc2nc3cc(C)c(CCCCCCCNc4cc5c(cc4C)nc4cc(C)c(NCCCCCCNc6cc7c(cc6C)nc6cc(C)c(N)cc6[n+]7-c6ccccc6)cc4[n+]5-c4ccccc4)cc3[n+](-c3ccccc3)c2cc1N]

### Example 5. Preparation of Branched Cationic Dye Multimers

Cationic dye moieties can be linked to form branched multimers using the scheme below, which illustrates preparation of a branched trimer comprising safranin-O moieties. The same process can be repeated as desired to prepare a cationic dye multimer comprising, e.g., 4, or 5 cationic dye moieties.

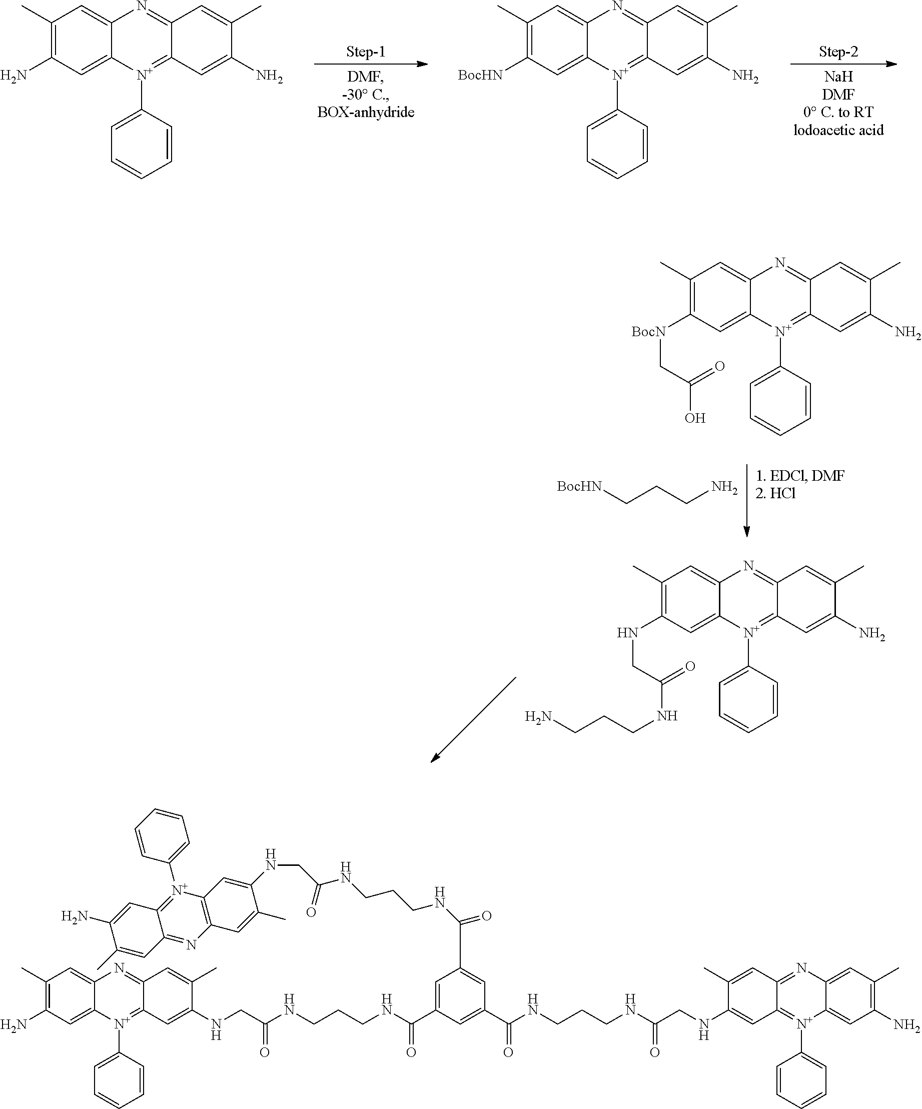 [SMILES: Cc1cc2nc3cc(C)c(N)cc3[n+](-c3ccccc3)c2cc1N ; Cc1cc2nc3cc(C)c(NC(=O)OC(C)(C)C)cc3[n+](-c3ccccc3)c2cc1N ; Cc1cc2nc3cc(C)c(N(CC(=O)O)C(=O)OC(C)(C)C)cc3[n+](-c3ccccc3)c2cc1N ; CC(C)(C)OC(=O)NCCCN ; Cc1cc2nc3cc(C)c(NCC(=O)NCCCN)cc3[n+](-c3ccccc3)c2cc1N ; Cc1cc2nc3cc(C)c(NCC(=O)NCCCNC(=O)c4cc(C(=O)NCCCNC(=O)CNc5cc6c(cc5C)nc5cc(C)c(N)cc5[n+]6-c5ccccc5)cc(C(=O)NCCCNC(=O)CNc5cc6c(cc5C)nc5cc(C)c(N)cc5[n+]6-c5ccccc5)c4)cc3[n+](-c3ccccc3)c2cc1N]

Tetramers can be prepared using a similar scheme and, for example, biphenyl-3,3′,5,5′-tetracarboxylic acid,

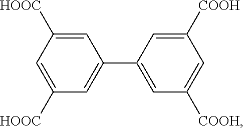 [SMILES: O=C(O)c1cc(C(=O)O)cc(-c2cc(C(=O)O)cc(C(=O)O)c2)c1]

instead of

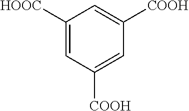 [SMILES: O=C(O)c1cc(C(=O)O)cc(C(=O)O)c1]

as used in the scheme above. Higher multimers can be prepared similarly, using the appropriate polycarboxylate linker.

### Example 6. Synthesis of Compound 1

### Synthesis of 7,7′-(hexane-1,6-diylbis(azanediyl))bis(3-amino-2,8-dimethyl-5-phenylphenazin-5-ium) chloride

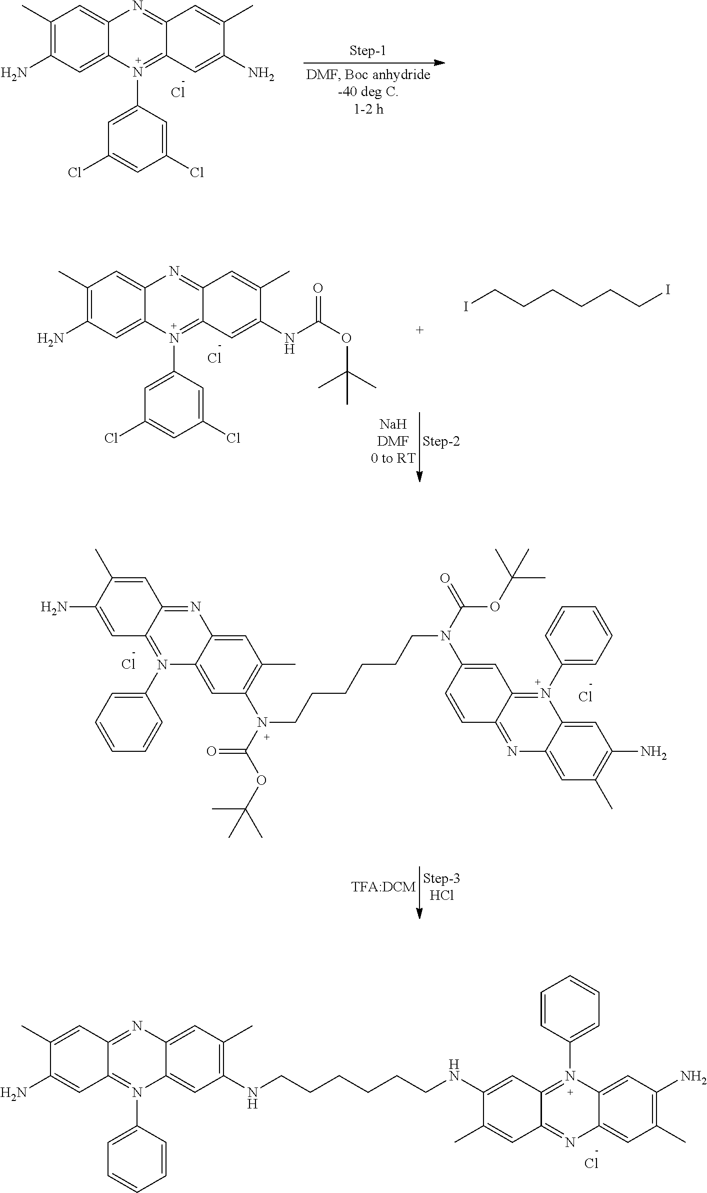

### Step-1: Synthesis of 3-amino-7-(tert-butoxycarbonylamino)-2,8-dimethyl-5-phenylphenazin-5-ium chloride

To a solution of Safranin-′O′ (5.2 g, 14.8 mmol) in DMF (50 mL), cooled to −40° C., was added sodium hydride (1.18 g, 29.5 mmol) and the reaction mass was stirred at the same temperature for 1 h. Boc anhydride (2.91 g, 13.97 mmol) was added at the same temperature and the reaction mass was stirred at the same temperature for another 1 h. The progress of the reaction was monitored by LCMS and TLC (System: 10% MeOH in DCM). The reaction was quenched by adding water (20 mL) and the solid obtained was filtered and purified by column chromatography (silica gel 100-200 mesh, eluent system-10% MeOH:DCM) to obtain 3-amino-7-(tert-butoxycarbonylamino)-2,8-dimethyl-5-phenylphenazin-5-ium chloride (1 g).

### Step-2: Synthesis of tert-butyl N-(8-amino-3,7-dimethyl-10-phenyl-phenazin-10-ium-2-yl)-N-[6-[(8-amino-3,7-dimethyl-10-phenyl-phenazin-10-ium-2-yl)-tert-butoxycarbonylamino]hexyl]carbamate dichloride

To a solution of 3-amino-7-(tert-butoxycarbonylamino)-2,8-dimethyl-5-phenylphenazin-5-ium chloride (225 mg, 0.5 mmol) in DMF (2.5 mL), cooled to −30° C., was added sodium hydride (40 mg, 1.0 mmol) and the reaction mass was stirred at the same temperature for 1 h. 1,6-Diiodohexane (85 mg, 0.25 mmol) was added and the reaction was stirred at the same temperature for 2 min and at 0° for 20 min. Additional 1,6-diiodohexane (33 mg, 0.097 mmol) was added and the reaction mixture was stirred at RT for 5 min. The reaction mixture was monitored by LCMS. After detection of product, the reaction was quenched with water (5 mL). The solid product obtained was filtered, dissolved in 20% MeOH/DCM solution (20 mL) and concentrated under reduced pressure to get 100 mg crude product, which was purified by reverse phase HPLC to get pure tert-butyl N-(8-amino-3,7-dimethyl-10-phenyl-phenazin-10-ium-2-yl)-N-[6-[(8-amino-3,7-dimethyl-10-phenyl-phenazin-10-ium-2-yl)-tert-butoxycarbonylamino]hexyl]carbamate dichloride (6 mg.)

### Step-3: Synthesis of 7,7′-(hexane-1,6-diylbis(azanediyl))bis(3-amino-2,8-dimethyl-5-phenyl phenazin-5-ium) chloride

7,7′-(2,2,15,15-Tetramethyl-4,13-dioxo-3,14-dioxa-5,12-diazahexadecane-5,12-diyl)bis(3-amino-2,8-dimethyl-5-phenylphenazin-5-ium) chloride (5 mg, 0.13 mmol) was dissolved in DCM (1 mL). Trifluoroacetic acid (1 mL) was added and the reaction was stirred at RT for 2 h. The desired product was detected by LCMS and NMR. The reaction mixture was concentrated under vacuum to obtain the desired product, which was triturated with diethyl ether and pentane. ¹HNMR (CD₃OD): δ (ppm): 7.90-7.70 (m, 10H), 7.55-7.50 (m, 4H), 6.05 (s, 2H), 5.73 (s, 2H), 3.05 (m, 4H), 2.4 (s, 6H), 2.35 (s, 6H), 1.55-1.42 (m, 6H), 1.22-1.15 (m, 6H). LCMS: 740(M+) and 370 (half fragment).

### Example 7. Synthesis of Compounds 2 and 3

This example provides a synthesis route for compounds 2 and 3. Appropriate starting materials can be obtained using the methods as presented in Example 2.

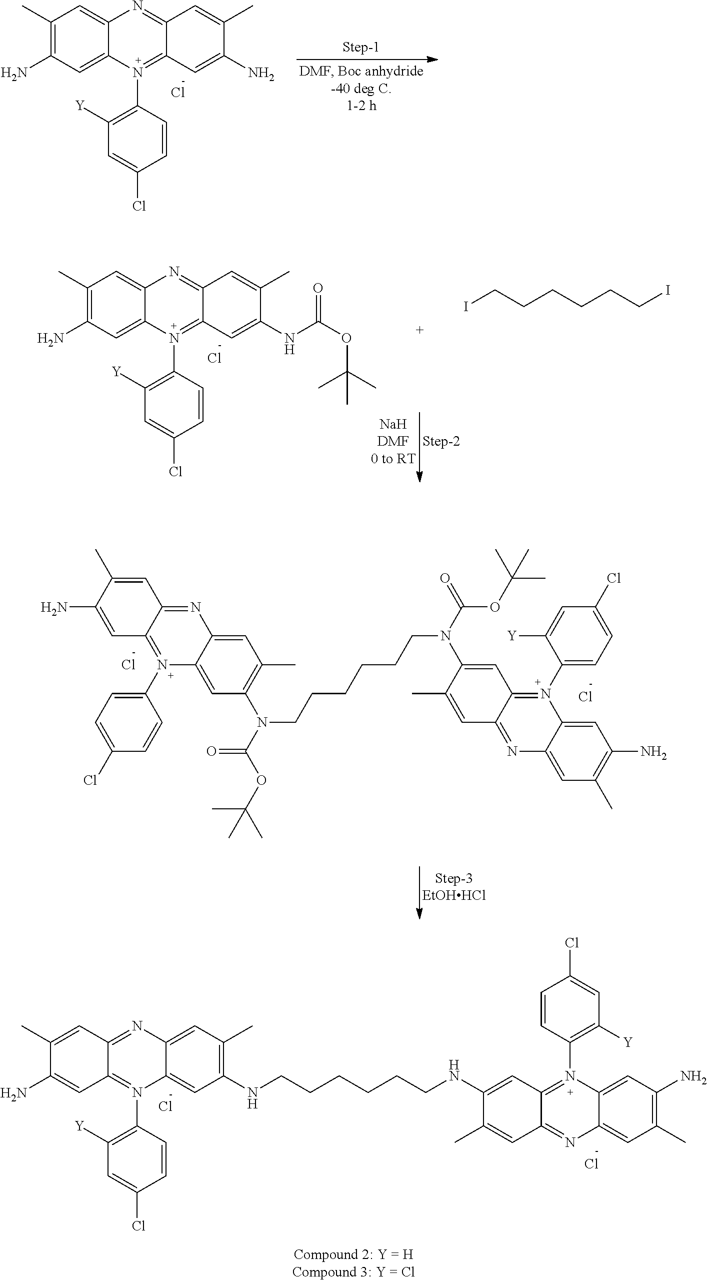

### Example 8. Synthesis of Compound 4

### Synthesis of 7,7′-(octane-1,8-diylbis(azanediyl))bis(3-amino-2,8-dimethyl-5-phenylphenazin-5-ium) chloride

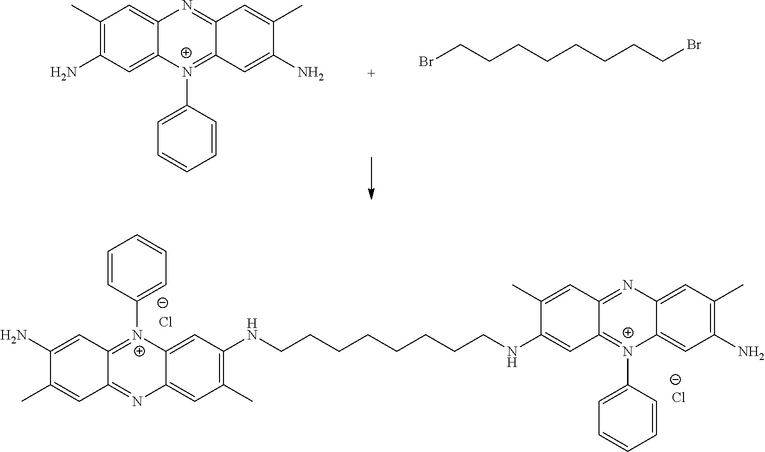 [SMILES: Cc1cc2nc3cc(C)c(N)cc3[n+](-c3ccccc3)c2cc1N ; BrCCCCCCCCBr ; Cc1cc2nc3cc(C)c(NCCCCCCCCNc4cc5c(cc4C)nc4cc(C)c(N)cc4[n+]5-c4ccccc4)cc3[n+](-c3ccccc3)c2cc1N ; [Cl-] ; [Cl-]]

To a stirred solution of Safranin-O (1.05 g, 3.0 mmol) in 15 mL of N,N-dimethylacetamide was added potassium carbonate (0.55 g, 4.0 mmol) and stirred at RT for 15 min. 1,8-Dibromooctane (272 mg, 1.0 mmol) was then added dropwise into the reaction mixture followed by the addition of potassium iodide (332 mg, 2.0 mmol). The reaction mixture was then stirred at 60° C. for 24 h under nitrogen. The reaction mixture was then allowed to cool to RT, 20 mL water was added and the crude reaction mixture was lyophilized. The mixture was then purified by column chromatography (silica gel, 100-200 mesh, 0-10% MeOH in DCM). The product obtained by column chromatography was re-purified by HPLC to afford the desired product as a brown solid. ¹H NMR (DMSO-d6): δ (ppm): 7.90-7.75 (m, 12H), 7.65-7.55 (m, 7H), 6.0 (s, 2H), 5.55 (s, 2H), 2.98 (m, 4H), 2.30 (s, 12H), 1.35 (m, 4H), 1.05 (bs, 8H). ¹HNMR (CD₃OD) δ (ppm): 7.90-7.70 (m, 10H), 7.55-7.50 (m, 4H), 6.05 (s, 2H), 5.73 (s, 2H), 3.05 (m, 4H), 2.4 (s, 6H), 2.35 (s, 6H), 1.55-1.42 (m, 6H), 1.22-1.15 (m, 6H). LCMS: 740 (M+) and 370 (half fragment).

### Example 9. Synthesis of Compound 6

### Synthesis of 3-amino-7-[(6-{[3,5-bis({6-[(7-amino-2,8-dimethyl-5-phenyl-5λ⁵,10-phenazin-5-ylium-3-yl)amino]hexyl}carbamoyl)phenyl]formamido}hexyl)amino]-2,8-dimethyl-5-phenyl-5λ⁵,10-phenazin-5-ylium

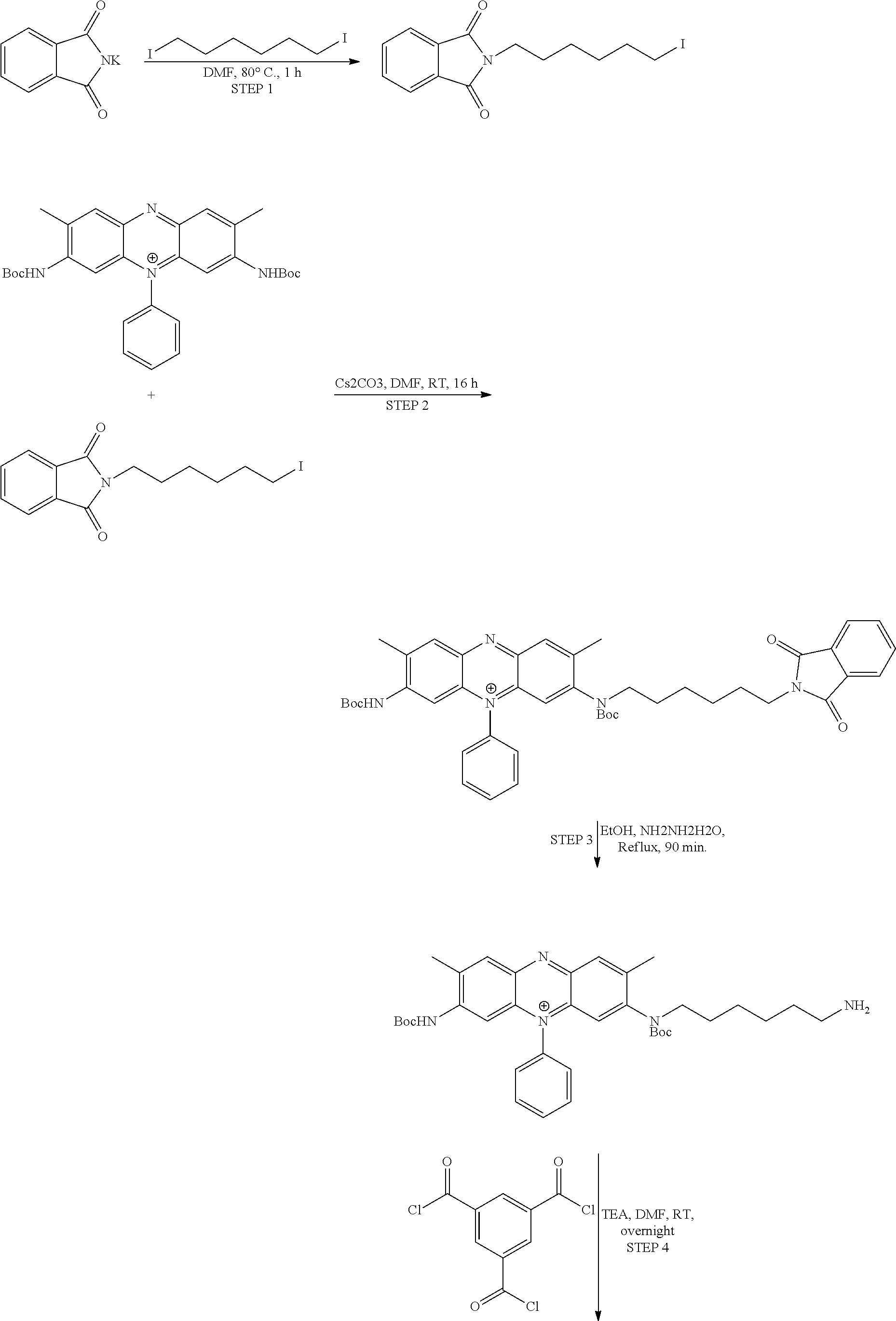

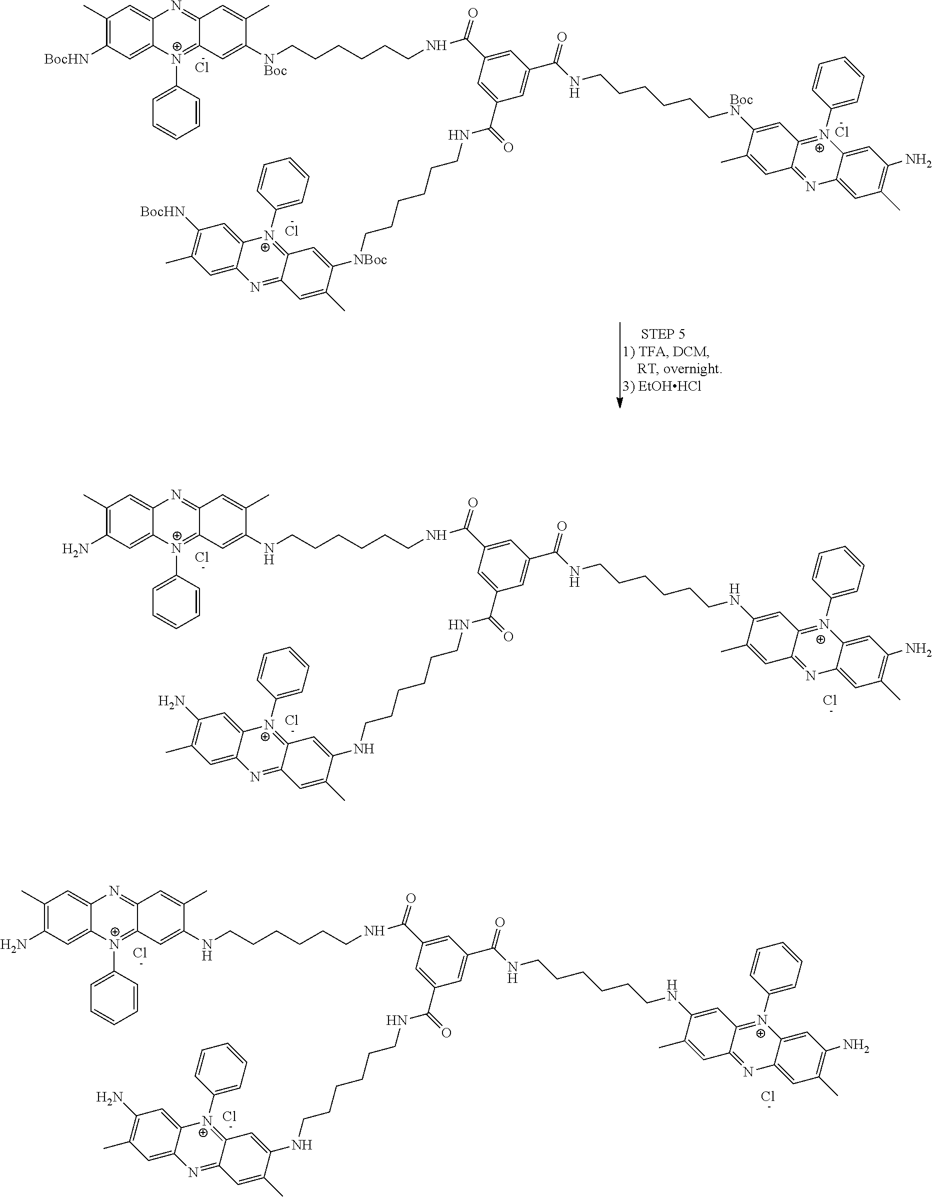

### Step-1: Synthesis of 2-(6-iodohexyl)isoindoline-1,3-dione

To a suspension of potassium phthalamide (700 mg, 3.7 mmol) in DMF (30 mL) was added 1,6-diiodohexane (3.83 g, 11.33 mmol) and the reaction mass was heated atw 80° C. for 1 h. (The reaction mass became clear on heating). The reaction mixture was cooled to RT, diluted with diethyl ether (100 mL) and water (100 mL), the organic layers were separated, and the aqueous layer was again extracted with diethyl ether (2×100 mL). The combined organic layer was washed with water (200 mL) and saturated brine solution (200 mL) The organic layer was dried over anhydrous sodium sulfate and concentrated under reduced pressure to obtain the product, which was purified by column chromatography (silica gel: 100-200 mesh; Eluent: 10% ethyl acetate in hexane) to obtain pure product as white solid (2.1 g).

### Step-2: Synthesis of tert-butyl N-[8-(tert-butoxycarbonylamino)-3,7-dimethyl-10-phenyl-phenazin-10-ium-2-yl]-N-[6-(1,3-dioxoisoindolin-2-yl)hexyl]carbamate

To a solution of 3,7-bis(tert-butoxycarbonylamino)-2,8-dimethyl-5-phenylphenazin-5-ium (500 mg, 0.90 mmol) in DMF (5 mL) was added cesium carbonate (1.48 g, 4.54 mmol) and the mixture stirred at RT for 10 min. 2-(6-Iodohexyl)isoindoline-1,3-dione (422 mg, 1.18 mmol) was added and the reaction mass was stirred at RT overnight. The reaction was monitored by LCMS and TLC. The reaction mass was diluted with EtOAc (25 mL) and water (25 mL), and the layers were separated. The aqueous layer was again extracted with EtOAc (2×25 mL). The combined organic layer was washed with water (2×100 mL), and saturated brine solution (200 mL), then dried over anhydrous sodium sulfate and concentrated under reduced pressure to obtain the product. This was purified by column chromatography by using neutral alumina and an eluent system of 0 to 2% Methanol in DCM) to obtain pure product (200 mg.)

### Step-3: Synthesis of tert-butyl N-(6-aminohexyl)-N-[8-(tert-butoxycarbonylamino)-3,7-dimethyl-10-phenyl-phenazin-10-ium-2-yl]carbamate

To a solution of 3-(tert-butoxycarbonyl(6-(1,3-dioxoisoindolin-2-yl)hexyl)amino)-7-(tert-butoxycarbonylamino)-2,8-dimethyl-5-phenylphenazin-5-ium (800 mg, 1.04 mmol) in ethanol (40 mL) was added hydrazine hydrate monohydrate (1.072 mg, 20.8 mmol) and the reaction mixture was heated to reflux for 90 min. The reaction was monitored by TLC and LCMS. The solvent was evaporated under reduced pressure to dryness. The residue was dissolved in diethyl ether (150 mL) and filtered, the filtrate was concentrated under reduced pressure to obtain the crude product which was purified by column chromatography using neutral alumina (Eluent System: 0 to 3% MeOH in DCM) to obtain pure product (410 mg).

### Step-4: Synthesis of tert-butyl N-[6-[[3,5-bis[6-[tert-butoxycarbonyl-[8-(tert-butoxycarbonylamino)-3,7-dimethyl-10-phenyl-phenazin-10-ium-2-yl]amino]hexylcarbamoyl]benzoyl]amino]hexyl]-N-[8-(tert-butoxycarbonylamino)-3,7-dimethyl-10-phenyl-phenazin-10-ium-2-yl]carbamate trichloride

To a solution of 3-((6-aminohexyl)(tert-butoxycarbonyl)amino)-7-(tert-butoxycarbonyl amino)-2,8-dimethyl-5-phenylphenazin-5-ium (243 mg, 0.395 mmol) in DMF (3 mL) was added DIPEA (233 mg, 1.80 mmol) and stirred at RT for 10 min. Benzene-1,3,5-tricarbonyl trichloride (30 mg, 0.113 mmol) was added and the reaction was stirred at RT overnight. The reaction was monitored by TLC and LCMS. The reaction mass was diluted with EtOAc (30 mL) and water (30 mL), and the organic layer was separated. The aqueous layer was again extracted with EtOAc (2×30 mL), the combined organic layer was washed with water (100 mL) and saturated brine solution (100 mL). The organic layer was dried over anhydrous sodium sulfate and concentrated under reduced pressure to obtain the product which was purified by column chromatography using neutral alumina (Eluent: 0.2-1% MeOH in DCM) to obtain pure product (65 mg.)

### Step-5: Synthesis of N1,N3,N5-tris[6-[(8-amino-3,7-dimethyl-10-phenyl-phenazin-10-ium-2-yl)amino]hexyl]benzene-1,3,5-tricarboxamide trichloride

To a solution of the diboc protected trimer (65 mg, 0.0325 mmol) in DCM (5 mL) was added trifluoroacetic acid (01 mL) and the reaction mixture was stirred at RT overnight. The reaction mixture was monitored by LCMS. The reaction mass was concentrated under reduced pressure to dryness and purified by reverse phase HPLC to obtain the product to obtain the product as the acetate counter anion. The product was dissolved in ethanol-HCl (20 mL) and concentrated to dryness (3 times), and the solid obtained was washed with water (20 mL) to remove inorganic impurities. The solid obtained was lyophilized to get the product (6.6 mg) which was confirmed by LCMS and NMR. ¹H NMR (CD₃OD): δ (ppm): 8.25 (s, 3H), 7.4-7.90 (m, 15H), 6.6 (s, 6H), 6.15 (s, 6H), 3.1-3.5 (m, 12H), 2.2-2.3 (m, 18H), 1.3-1.9 (m, 24H). M+(1397), M/2 (699.1), M/3 (467.1).

### Example 10. Synthesis of Compounds 18 and 19

### Synthesis of 3-amino-7-{[(2-{[(7-amino-2,8-dimethyl-5-phenyl-5λ⁵,10-phenazin-5-ylium-3-yl)amino]methyl}phenyl)methyl]amino}-2,8-dimethyl-5-phenyl-5λ⁵,10-phenazin-5-ylium and 3-amino-7-{[(2-{[(7-amino-2,8-dimethyl-5-phenyl-5λ⁵,10-phenazin-5-ylium-3-yl)amino]methyl}phenyl)methyl]amino}-2,6-dimethyl-5-phenyl-5λ⁵,10-phenazin-5-ylium

### Step-1: Synthesis of 7,7′-(1,2-phenylenebis(methylene))bis(tert-butoxycarbonylazanediyl)bis(3-(tert-butoxycarbonylamino)-2,8-dimethyl-5-phenylphenazin-5-ium) chloride & 7-(tert

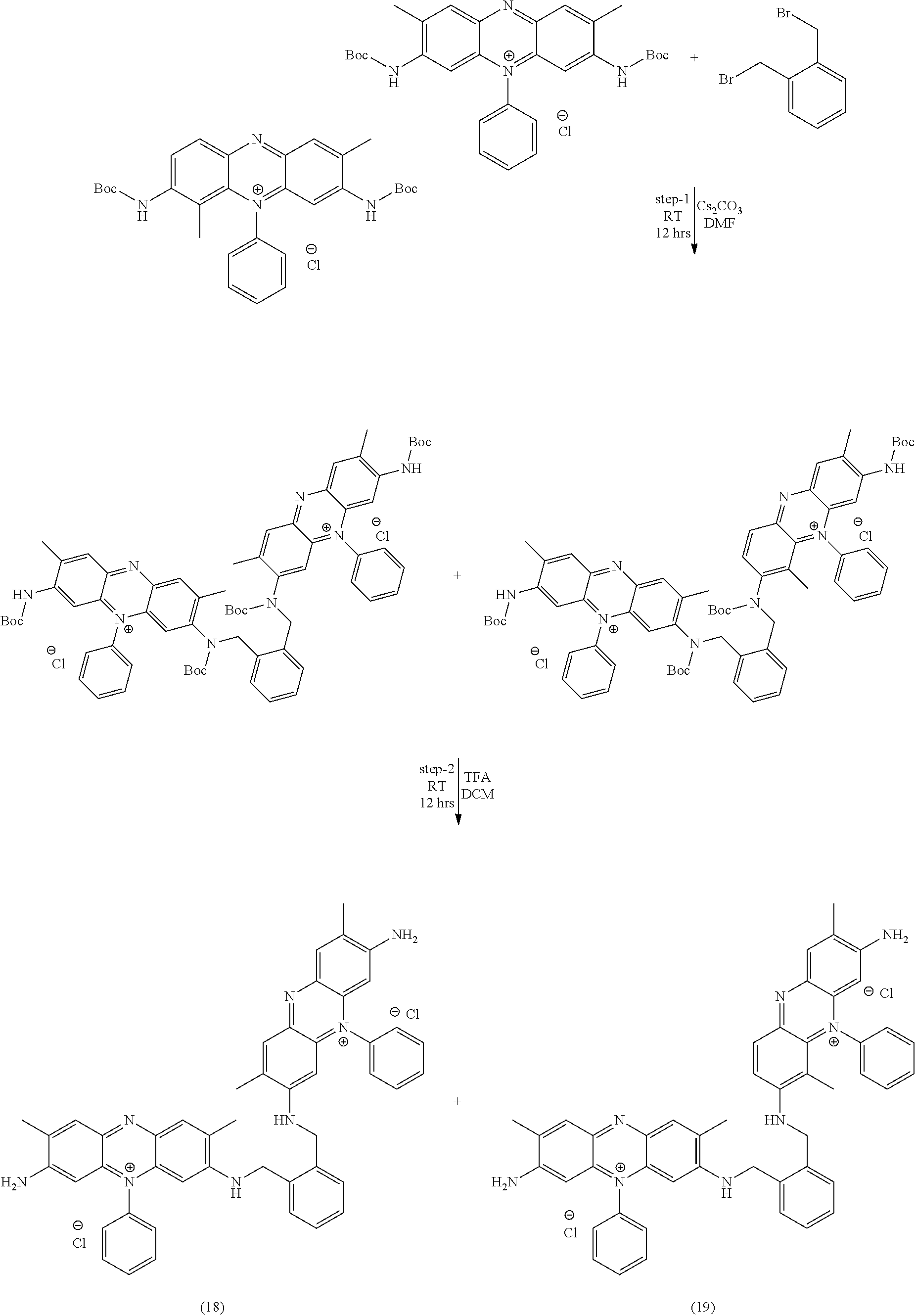

### xycarbonyl(2-((tert-butoxycarbonyl(7-(tert-butoxycarbonylamino)-2,8-dimethyl-5-phenylphenazin-5-ium-3-yl)amino)methyl)benzyl)amino)-3-(tert-butoxycarbonylamino)-2,6-dimethyl-5-phenylphenazin-5-ium

To a stirred solution of 3,7-bis(tert-butoxycarbonylamino)-2,8-dimethyl-5-phenylphenazin-5-ium chloride & 3,7-bis(tert-butoxycarbonylamino)-2,6-dimethyl-5-phenylphenazin-5-ium chloride (500 mg, 0.90 mmol) in 5 mL of DMF was added cesium carbonate (880 mg, 2.70 mmol) and the mixture stirred at RT for 15 min. The reaction mixture was then cooled to 0° C. and o-xylene dibromide (132 mg, 0.50 mmol, diluted with 1 ml of DMF) was added dropwise. The reaction mixture was stirred at RT for 12 h. The reaction was monitored by TLC and LCMS. After completion of reaction, mixture was diluted with ice-cold water and filtered. The solid residue obtained was dissolved in diethyl ether (50 mL) and washed with brine solution (3×20 mL). The organic layer was dried over anhydrous sodium sulfate, and concentrated under reduced pressure to afford crude product which was then purified by column chromatography (neutral alumina, eluent 0-20% EtOAc in hexane) to afford the desired product as a dark brown solid (250 mg).

### Step-2: Synthesis of 7,7′-(1,2-phenylenebis(methylene))bis(azanediyl)bis(3-amino-2,8-dimethyl-5-phenylphenazin-5-ium) chloride & 3-amino-7-(2-((7-amino-2,8-dimethyl-5-phenylphenazin-5-ium-3-ylamino)methyl)benzylamino)-2,6-dimethyl-5-phenylphenazin-5-ium chloride

To a stirred solution of 7,7′-(1,2-phenylenebis(methylene))bis(tert-butoxycarbonylazanediyl)-bis(3-(tert-butoxycarbonylamino)-2,8-dimethyl-5-phenylphenazin-5-ium) chloride (250 mg, 0.27 mmol) in 5 mL of DCM was added TFA (1 mL) at 0° C. dropwise and the mixture stirred at RT for 12 h. The reaction was monitored by TLC and LCMS. After completion of reaction, the solvent was removed under reduced pressure to dryness and the residue purified by reverse phase HPLC to afford two peaks of the same mass. Both peaks were then treated separately with methanolic HCl and dried, then washed with water and dried again to afford desired product. (Compound 18): 8 mg. (Compound 19): 8 mg. ¹H NMR Compound 18: (DMSO-d6): δ (ppm): 8.15 (t, 2H), 7.90 (d, 4H), 7.85-7.75 (m, 6H), 7.6 (m, 3H), 7.40 (d, 4H), 7.05 (m, 2H), 6.65 (m, 2H), 5.95 (s, 2H), 5.45 (s, 2H), 4.10 (m, 4H), 2.40 (s, 6H), 2.30 (s, 6H). LCMS: 732 (M+), 366 (M/2). Compound 19: (DMSO-d6): δ (ppm): 8.15 (bs, 1H), 7.95 (s, 1H), 7.90-7.75 (m, 5H), 7.74-7.60 (m, 7H), 7.42 (d, 2H), 7.20-7.10 (m, 2H), 7.05 (t, 1H), 6.65 (d, 1H), 6.10 (s, 1H), 5.95 (s, 1H), 5.62 (s, 1H), 4.58 (m, 2H), 4.40 (m, 2H), 2.40 (s, 3H), 2.30 (s, 6H), 1.48 (s, 3H). LCMS: 732 (M+), 366 (M/2).

### Example 11. Synthesis of Compound 20

### Synthesis of (3-amino-7-{[6-({4-[4-({6-[(7-amino-5-chloranuide-2,8-dimethyl-5-phenyl-5λ⁶,10-phenazin-5-ylium-3-yl)amino]hexyl}carbamoyl)phenyl]phenyl}formamido)hexyl]amino}-2,8-dimethyl-5-phenyl-5λ⁶,10-phenazin-5-ylium-5-e)chloranuide

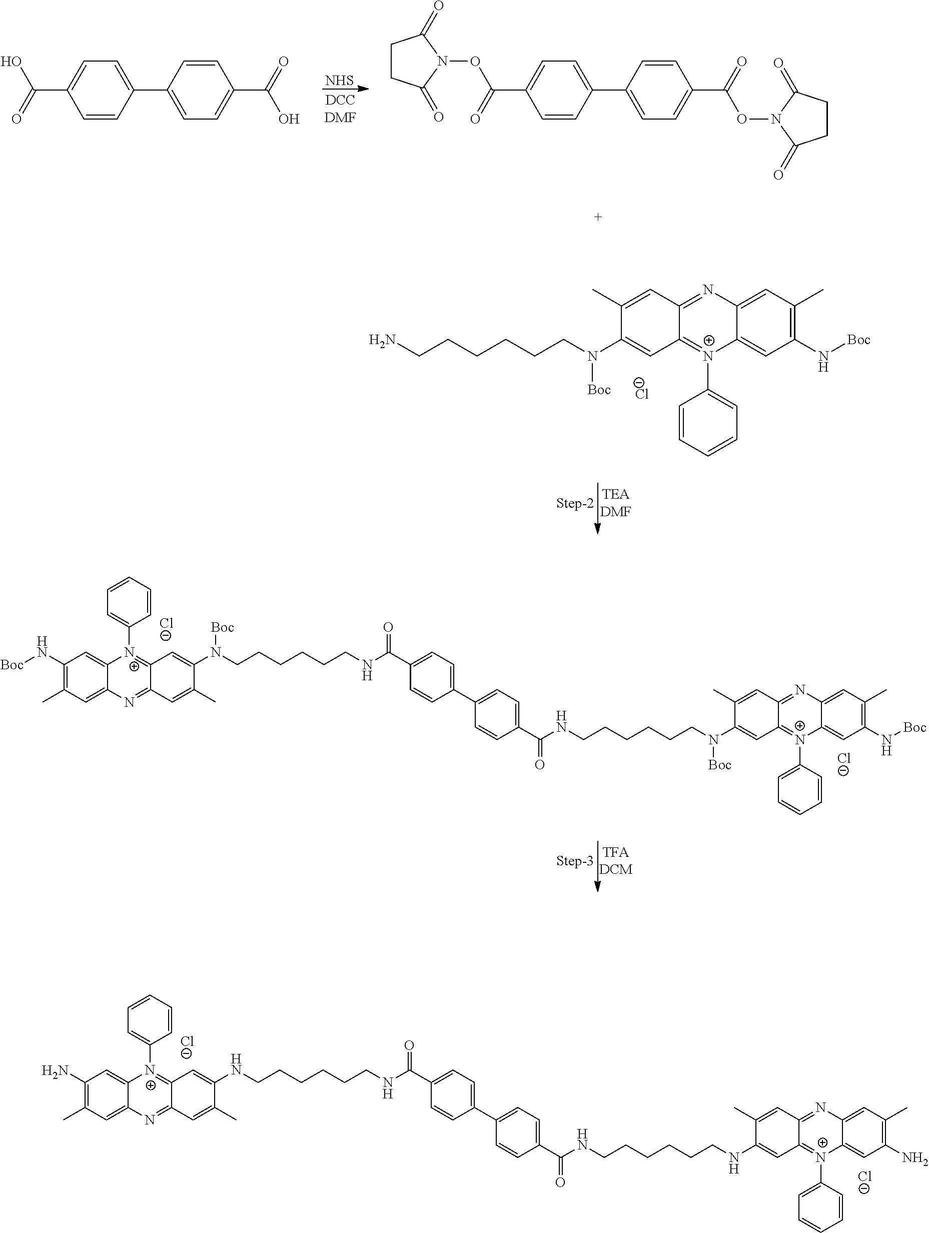

### Step-1: Synthesis of bis(2,5-dioxopyrrolidin-1-yl) biphenyl-4,4′-dicarboxylate

To a stirred solution of biphenyl-4,4′-dicarboxylic acid (500 mg, 2.06 mmol) in 10 mL of DMF was added N-hydroxysuccinimide (594 mg, 5.16 mmol) and dicyclohexylcarbodiimide (1.06 g, 5.16 mmol) at 0° C. and the mixture stirred at RT overnight. The reaction mixture was filtered and the filtered cake was washed with EtOAc. The washings and filtrate were combined, washed with brine solution (3×20 mL), dried over anhydrous sodium sulfate and concentrated under reduced pressure to afford a crude product that was dissolved in DCM and again filtered. The DCM layer was concentrated under reduced pressure to afford desired product (600 mg).

### Step-2: Synthesis of tert-butyl N-[8-(tert-butoxycarbonylamino)-3,7-dimethyl-10-phenyl-phenazin-10-ium-2-yl]-N-[6-[[4-[4-[6-[tert-butoxycarbonyl-[8-(tert-butoxycarbonylamino)-3,7-dimethyl-10-phenyl-phenazin-10-ium-2-yl]amino]hexylcarbamoyl]phenyl]benzoyl]amino]hexyl]carbamate dichloride

Bis(2,5-dioxopyrrolidin-1-yl)biphenyl-4,4′-dicarboxylate (20 mg, 0.045 mmol) and 3-((6-aminohexyl)(tert-butoxycarbonyl)amino)-7-(tert-butoxycarbonylamino)-2,8-dimethyl-5-phenylphenazin-5-ium chloride (65.5 mg, 0.100 mmol) were dissolved in 2 mL of DMF and triethylamine (14 mg) was added. The reaction mixture was stirred at RT for 16 h. After completion of reaction, the mixture was diluted with ice-cold water and extracted with EtOAc. The organic layer was washed with brine solution (4×10 mL), dried over anhydrous sodium sulfate and concentrated under reduced pressure to afford the crude product, which was purified by column chromatography (neutral alumina) to afford the desired product (70 mg).

### Step-3: Synthesis of N-[6-[(8-amino-3,7-dimethyl-10-phenyl-phenazin-10-ium-2-yl)amino]hexyl]-4-[4-[6-[(8-amino-3,7-dimethyl-10-phenyl-phenazin-10-ium-2-yl)amino]hexylcarbamoyl]phenyl]benzamide dichloride

The product from Step-2 (70 mg) was dissolved in 2 mL of DCM and TFA (0.5 mL) was added at 0° C. dropwise and stirred at RT for 12 h. The reaction was monitored by TLC and LCMS. After completion of reaction, solvent was removed under reduced pressure to dryness to obtained crude product which was purified by reverse phase HPLC. The product obtained after purification was then treated with Methanolic HCl and dried and then washed with water and freeze dried to afford the desired product (6 mg). ¹H NMR (DMSO-d6): δ (ppm): 8.56 (t, 2H), 7.97-7.95 (d, 4H), 7.90-7.75 (m, 12H), 7.70-7.65 (m, 2H), 7.62 (d, 4H), 7.60-7.45 (m, 3H), 6.02 (s, 2H), 5.58 (s, 2H), 3.25 (m, 4H), 2.98 (m, 4H), 2.35 (s, 6H), 2.33 (s, 6H), 1.50-1.42 (m, 4H), 1.40-1.30 (m, 4H), 1.20-1.05 (m, 8H).

### Example 12. Synthesis of Compound 21

### Synthesis of (3-amino-7-{10-[(7-amino-5-chloranuide-2,8-dimethyl-5-phenyl-5λ⁶,10-phenazin-5-ylium-3-yl)carbamoyl]decanamido}-2,8-dimethyl-5-phenyl-5λ⁶,10-phenazin-5-ylium-5-e)chloranuide

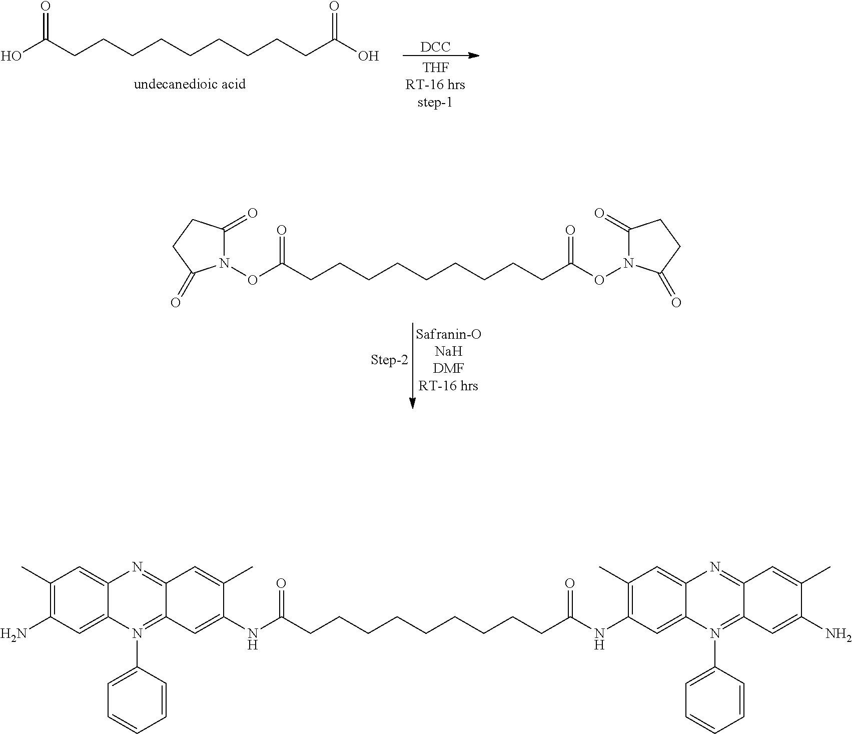

### Step-1: Synthesis of bis(2,5-dioxopyrrolidin-1-yl) undecanedioate

To a stirred solution of undecanedioic acid (500 mg, 2.31 mmol) in 10 mL of anhydrous THF was added N-hydroxysuccinimide (585 mg, 5.09 mmol) and dicyclohexylcarbodiimide (1.08 g, 5.09 mmol) at 0° C. and then allowed to stir at RT for 16 h. The reaction mixture was filtered and the filtrate was concentrated under reduced pressure. The product obtained was washed with diethyl ether and dried to afford the desired product (725 mg).

### Step-2: Synthesis of (3-amino-7-{10-[(7-amino-5-chloranuide-2,8-dimethyl-5-phenyl-5λ⁶,10-phenazin-5-ylium-3-yl)carbamoyl]decanamido}-2,8-dimethyl-5-phenyl-5λ⁶,10-phenazin-5-ylium-5-e)chloranuide

To a stirred solution of Safranin-O (350 mg, 1.0 mmol) in 10 mL of DMF was added sodium hydride (60 mg, 1.5 mmol) at 0° C. and stirred for 10 min, bis(2,5-dioxopyrrolidin-1-yl) undecanedioate (205 mg, 0.5 mmol) was added and allow to stir at RT for 16 h. After completion of reaction, the mixture was diluted with ice-cold water and filtered. The product obtained was dried and purified by reverse phase HPLC. The purified product was treated with Methanolic HCl (5 times), dried and washed with water and freeze dried to afford the desired product (5 mg). ¹H NMR (CD₃OD): δ (ppm): 8.56 (s, 1H), 8.18 (s, 2H), 7.97-7.95 (m, 3H), 7.90-7.80 (m, 6H), 7.60-7.50 (m, 4H), 6.18 (s, 2H), 2.55 (s, 6H), 2.45-2.40 (m, 10H), 1.65-1.58 (m, 4H), 1.35-1.25 (m, 10H).

### Example 13. Synthesis of Compounds 22 and 23

### Synthesis of [3-amino-7-({9-[(7-amino-5-chloranuide-2,8-dimethyl-5-phenyl-5λ⁶,10-phenazin-5-ylium-3-yl)amino]nonyl}amino)-2,8-dimethyl-5-phenyl-5λ⁶,10-phenazin-5-ylium-5-e]chloranuide and [3-amino-7-({9-[(7-amino-5-chloranuide-4,8-dimethyl-5-phenyl-5λ⁶,10-phenazin-5-ylium-3-yl)amino]nonyl}amino)-2,8-dimethyl-5-phenyl-5λ⁶,10-phenazin-5-ylium-5-e]chloranuide

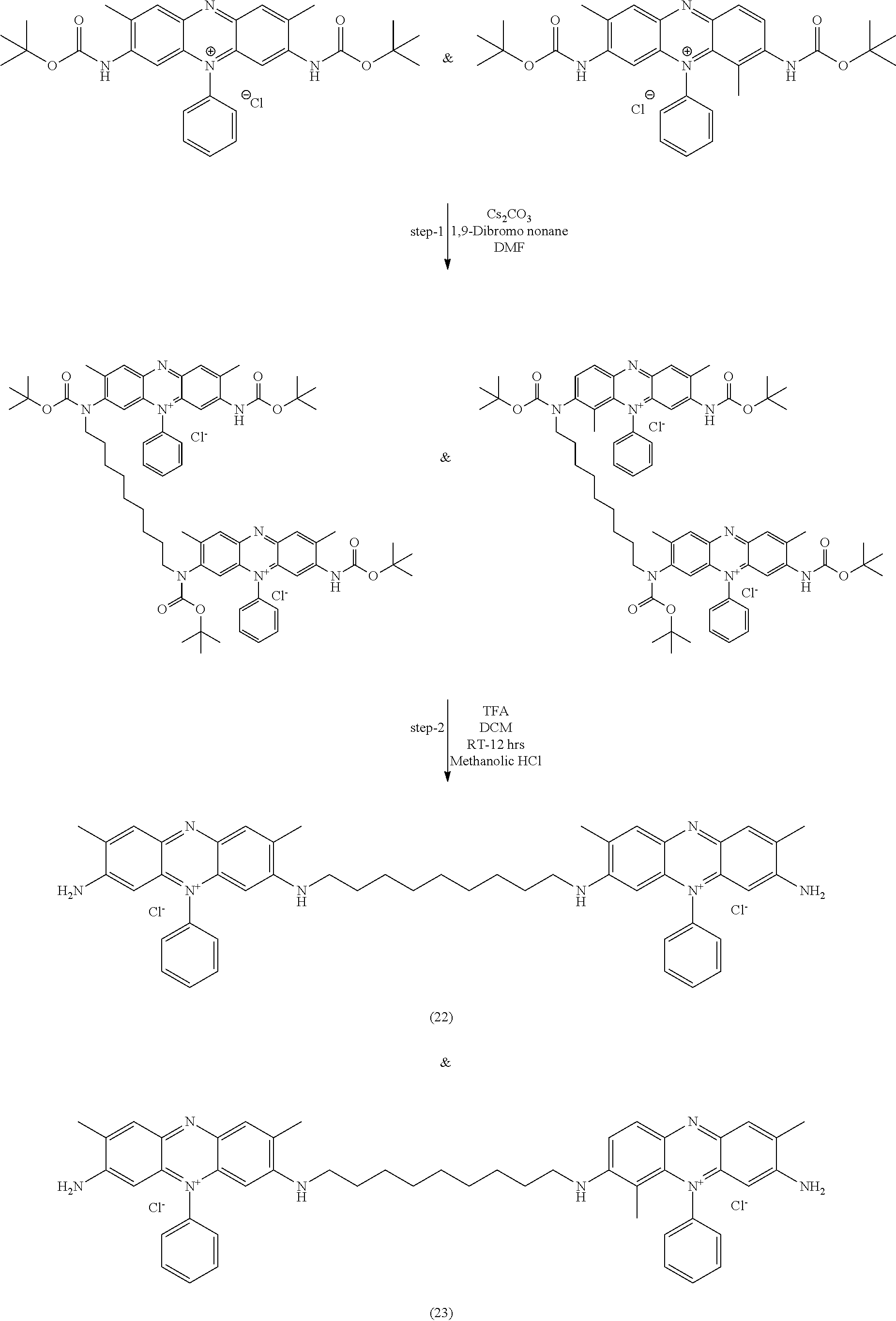

### Step-1: Synthesis of 7,7′-(2,2,18,18-tetramethyl-4,16-dioxo-3,17-dioxa-5,15-diazanonadecane-5,15-diyl)bis(3-(tert-butoxycarbonylamino)-2,8-dimethyl-5-phenylphenazin-5-ium) chloride and 7-(tert-butoxycarbonyl(9-(tert-butoxycarbonyl(7-(tert-butoxycarbonylamino)-2,8-dimethyl-5-phenylphenazin-5-ium-3-yl)amino)nonyl)amino)-3-(tert-butoxycarbonylamino)-2,6-dimethyl-5-phenylphenazin-5-ium chloride

To a stirred solution of mixture of 3,7-bis(tert-butoxycarbonylamino)-2,8-dimethyl-5-phenylphenazin-5-ium chloride and 3,7-bis(tert-butoxycarbonylamino)-2,6-dimethyl-5-phenylphenazin-5-ium chloride (500 mg, 0.909 mmol) in 10 mL of DMF was added cesium carbonate (738 mg, 2.27 mmol) and stirred at RT for 15 min. The reaction mixture was cooled to 0° C. and 1,9-dibromononane (156 mg, 0.545 mmol, diluted with 1 mL of DMF) was added dropwise. The reaction mixture was stirred at RT for 12 h. The reaction was monitored by TLC and LCMS. After completion of reaction, the mixture was diluted with ice-cold water and filtered. The solid residue obtained after filtration was dissolved in diethyl ether (200 mL) and washed with brine solution (3×50 mL). The ether layer was dried over anhydrous sodium sulfate, concentrated under reduced pressure to afford the crude product which was purified by column chromatography (neutral alumina, eluent 0-20% EtOAc in hexane) to afford the desired product (mixture of 2 products of same mass) as a dark brown solid (400 mg).

### Step-2: Synthesis of 7,7′-(nonane-1,9-diylbis(azanediyl))bis(3-amino-2, 8-dimethyl-5-phenylphenazin-5-ium) chloride and 3-amino-7-(9-(7-amino-2,8-dimethyl-5-phenylphenazin-5-ium-3-ylamino)nonylamino)-2,6-dimethyl-5-phenylphenazin-5-ium chloride

To a stirred solution of mixture of 7,7′-(2,2,18,18-tetramethyl-4,16-dioxo-3,17-dioxa-5,15-diazanonadecane-5,15-diyl)bis(3-(tert-butoxycarbonylamino)-2,8-dimethyl-5-phenylphenazin-5-ium) chloride and 7-(tert-butoxycarbonyl(9-(tert-butoxycarbonyl(7-(tert-butoxycarbonyl amino)-2,8-dimethyl-5-phenylphenazin-5-ium-3-yl)amino)nonyl)amino)-3-(tert-butoxy carbonylamino)-2,6-dimethyl-5-phenylphenazin-5-ium chloride (400 mg, 0.32 mmol) in 5 mL of DCM was added TFA (2 mL) at 0° C. dropwise and stirred at RT for 12 hrs. The reaction was monitored by TLC and LCMS. After completion of reaction, solvent was removed under reduced pressure to dryness to obtained crude product which was purified by reverse phase HPLC to afford two peaks of same mass. Both peaks were then treated separately with Methanolic HCl and dried, then washed with water and dried again to afford the desired products. Compound 22: 35 mg. Compound 23: 5 mg. ¹H NMR Compound 22: (DMSO-d6): δ (ppm): 7.90-7.78 (m, 10H), 7.75-7.70 (m, 3H), 7.65-7.55 (m, 7H), 6.02 (s, 2H), 5.58 (s, 2H), 2.98 (m, 4H), 2.35 (s, 6H), 2.33 (s, 6H), 1.40-1.30 (m, 4H), 1.20-1.03 (m, 10H). Compound 23: (DMSO-d6): δ (ppm): 7.95 (d, 1H), 7.90-7.60 (m, 13H), 7.60-7.38 (m, 3H), 6.15 (s, 1H), 6.0 (s, 1H), 5.54 (s, 1H), 3.42 (m, 2H), 2.98 (m, 2H), 2.30 (m, 9H), 1.60 (m, 2H), 1.40 (s, 3H), 1.38-1.20 (m, 8H), 1.18-1.02 (m, 4H).

### Example 14. Synthesis of Compound 24

### Synthesis of {3-amino-2,8-dimethyl-5-phenyl-7-[(6-{[3,5,7-tris({6-[(7-amino-5-chloranuide-2,8-dimethyl-5-phenyl-5λ⁶,10-phenazin-5-ylium-3-yl)amino]hexyl}carbamoyl)adamantan-1-yl]formamido}hexyl)amino]-5λ⁶,10-phenazin-5-ylium-5-e}chloranuide

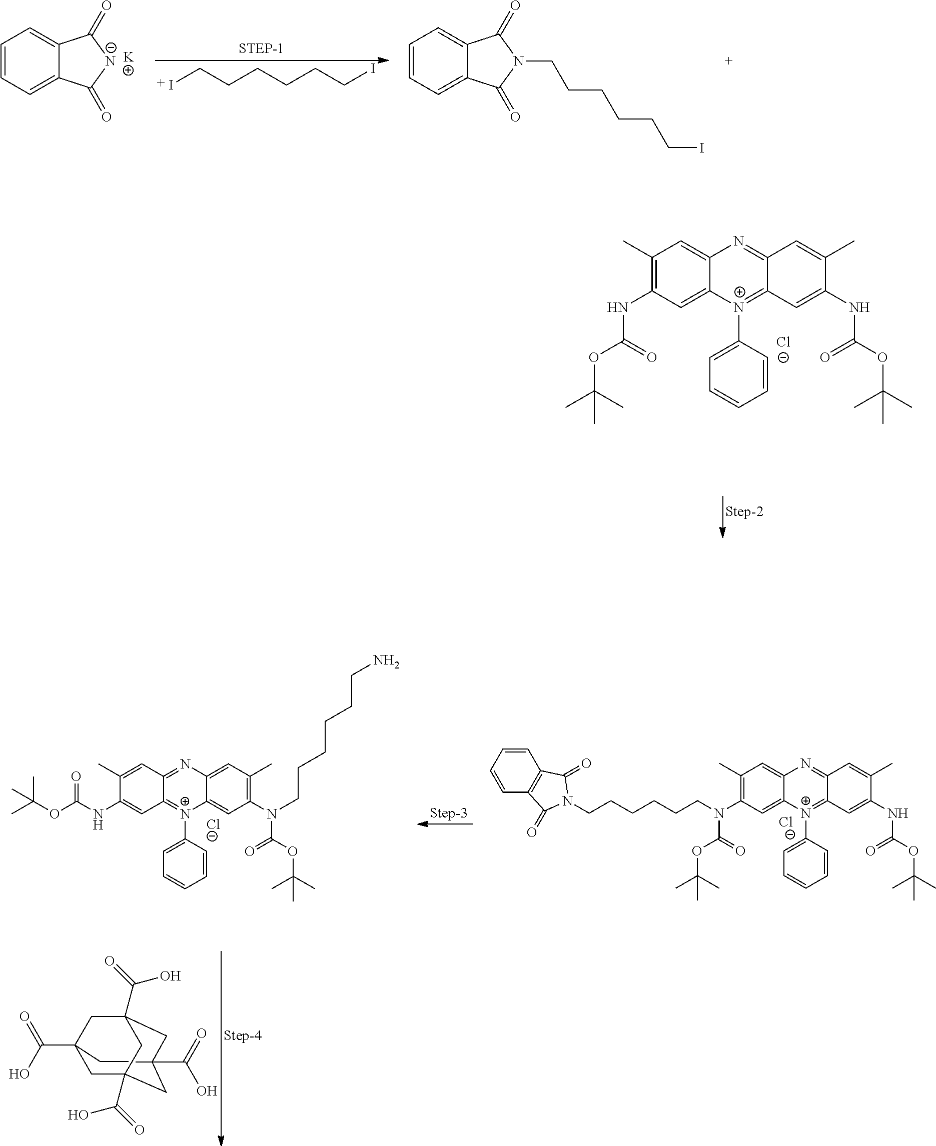

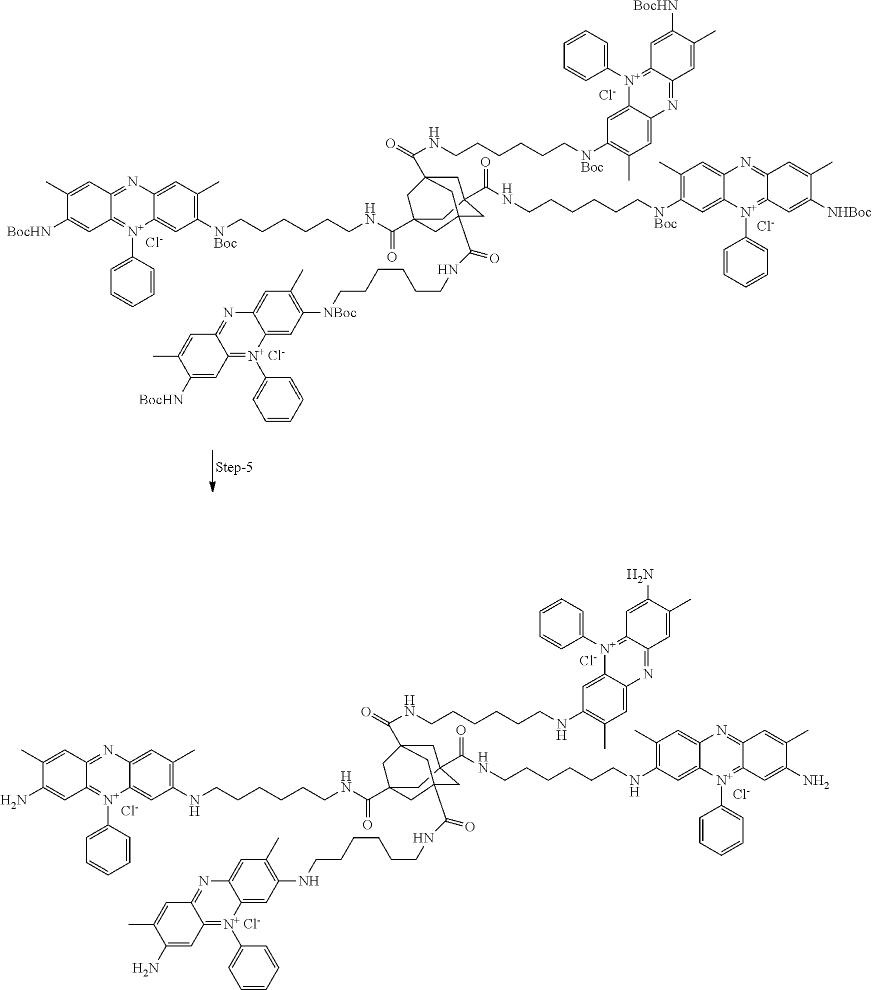

### Step-1:—Synthesis of 2-(6-iodohexyl)isoindoline-1,3-dione

To a suspension of potassium 1,3-dioxoisoindolin-2-ide (5 g, 27 mmol) in DMF (250 mL) was added 1,6-diiodohexane (22.83 g, 67.5 mmol) dropwise and heated at 85° C. for 2 h. The reaction mixture was monitored with LCMS. The reaction mixture was diluted with cold water and extracted with diethyl ether (2×300 mL), and the combined organic layer was washed with water (3×250 mL) and brine (250 mL). The organic layer dried over sodium sulfate and concentrated under reduced pressure to obtain a crude product which was purified by column chromatography with 10% EtOAc in hexane as eluent to afford 8.5 g of 2-(6-iodohexyl)isoindoline-1,3-dione.

### Step-2:—Synthesis of 3-(tert-butoxycarbonyl(6-(1,3-dioxoisoindolin-2-yl)hexyl)amino)-7-(tert-butoxycarbonylamino)-2,8-dimethyl-5-phenylphenazin-5-ium chloride

To a solution of 3,7-bis(tert-butoxycarbonylamino)-2,8-dimethyl-5-phenylphenazin-5-ium chloride (6 g, 10.90 mmol) in DMF (80 mL) at RT was added cesium carbonate (8.85 g, 27.25 mmol) portion-wise and stirred for 15 min. Then, 2-(6-iodohexyl)isoindoline-1,3-dione (5.06 g, 14.18 mmol) was dissolved in DMF (40 mL) and added dropwise at 0° C. The reaction mixture was stirred at RT for 16 h. The reaction mixture was monitored by LCMS and diluted with cold water and extracted with EtOAc (2×200 mL). The combined organic layer washed with brine (4×100 mL), dried over sodium sulfate, and concentrated under reduced pressure. The crude product was purified by column chromatography (Neutral alumina eluent 40% EtOAc in hexane to afford 6.5 g of 3-(tert-butoxycarbonyl(6-(1,3-dioxoisoindolin-2-yl)hexyl)amino)-7-(tert-butoxycarbonylamino)-2,8-dimethyl-5-phenylphenazin-5-ium chloride.

### Step-3:—Synthesis of 3-((6-aminohexyl)(tert-butoxycarbonyl)amino)-7-(tert-butoxycarbonylamino)-2,8-dimethyl-5-phenylphenazin-5-ium chloride

To a solution of 3-(tert-butoxycarbonyl(6-(1,3-dioxoisoindolin-2-yl)hexyl)amino)-7-(tert-butoxycarbonylamino)-2,8-dimethyl-5-phenylphenazin-5-ium chloride (4 g, 5.12 mmol) in ethanol (250 mL) was added hydrazine hydrate (5.36 g, 107.38 mmol) and heated to reflux at 85° C. for 1 h. The reaction mixture was monitored by LCMS and then the reaction mixture was concentrated and the residue dissolved in diethyl ether and filtered. The filtrate was concentrated under reduced pressure and the crude product was purified by column chromatography (Neutral alumina eluent 4% MeOH in DCM) to afford 1.2 g of 3-((6-aminohexyl)(tert-butoxycarbonyl)amino)-7-(tert-butoxycarbonylamino)-2, 8-dimethyl-5-phenylphenazin-5-ium chloride.

### Step-4:—Synthesis of 3-[(tert-butoxycarbonyl)(6-{[3,5,7-tris({6-[(tert-butoxycarbonyl)({7-[(tert-butoxycarbonyl)amino]-2,8-dimethyl-5-phenyl-5λ⁵-phenazin-5-ylium-3-yl})amino]hexyl}carbamoyl)adamantan-1-yl]formamido}hexyl)amino]-7-[(tert-butoxycarbonyl)amino]-2,8-dimethyl-5-phenyl-5λ⁵-phenazin-5-ylium tetrachloride

To a solution of adamantine-1,3,5,7-tetracarboxylic acid (30 mg, 0.0961 mmol) in DMF (6 mL) were added EDC.HCl (137 mg, 0721 mmol) and HOBt (97 mg, 0.721 mmol) and the mixture stirred at RT for 15 min. Then 3-((6-aminohexyl)(tert-butoxycarbonyl)amino)-7-(tert-butoxycarbonylamino)-2,8-dimethyl-5-phenylphenazin-5-ium chloride (280 mg, 0.432 mmol) and DIPEA (0.23 mL, 1.345 mmol) were added portion-wise and the mixture stirred at RT for 16 h. The reaction mixture was monitored by LCMS and diluted with water. The precipitate was filtered and washed with water. The residue was dissolved in DCM (75 mL), dried over sodium sulfate and concentrated under reduced pressure to obtain the crude product, which was purified by column chromatography (Neutral alumina eluent 5% MeOH in DCM) to afford 120 mg of BOC protected tetramer of adamantine.

### Step-5:—Synthesis of 2-{3-amino-2,8-dimethyl-7-[(6-{[3,5,7-tris({6-[(7-amino-2,8-dimethyl-5-phenyl-5λ⁵-phenazin-5-ylium-3-yl)amino]hexyl}carbamoyl)adamantan-1-yl]formamido}hexyl)amino]-5λ⁵-phenazin-5-ylium-5-yl}benzen-1-ide trichloride hydrochloride

To the BOC protected tetramer of adamantine from Step-4 (120 mg, 0.046 mmol) in DCM (4 mL) at 0° C. was added TFA (0.1 mL) dropwise and the mixture stirred at RT for 16 h. The reaction mixture was monitored by LCMS and solvent was removed and the crude product was purified by HPLC. The product was treated with Methanolic HCl (4×20 mL) and concentrated each time, then triturated with water (5 mL), filtered, and washed with water (20 mL). The residue was dried under lyophilization to afford 25 mg of desired product. 1H NMR (DMSO): δ (ppm): 7.60-7.90 (m, 44H), 6.02 (s, 4H), 5.45 (s, 1H), 2.90-3.0 (m, 16H), 2.30 (s, 12H), 2.25 (s, 12H), 1.76 (s, 12H), 1.30-1.36 (m, 16H), 1.05-1.10 (m, 16H). LCMS: 1897.10, M/4=474.6, M/3=632.6, M/2=948.5.

### Example 15. Synthesis of Compound 25

### Synthesis of {3-amino-2,8-dimethyl-5-phenyl-7-[(3-{[3,5,7-tris({3-[(7-amino-5-chloranuide-2,8-dimethyl-5-phenyl-5λ⁶,10-phenazin-5-ylium-3-yl)amino]propyl}carbamoyl)adamantan-1-yl]formamido}propyl)amino]-5λ⁶,10-phenazin-5-ylium-5-e}chloranuide

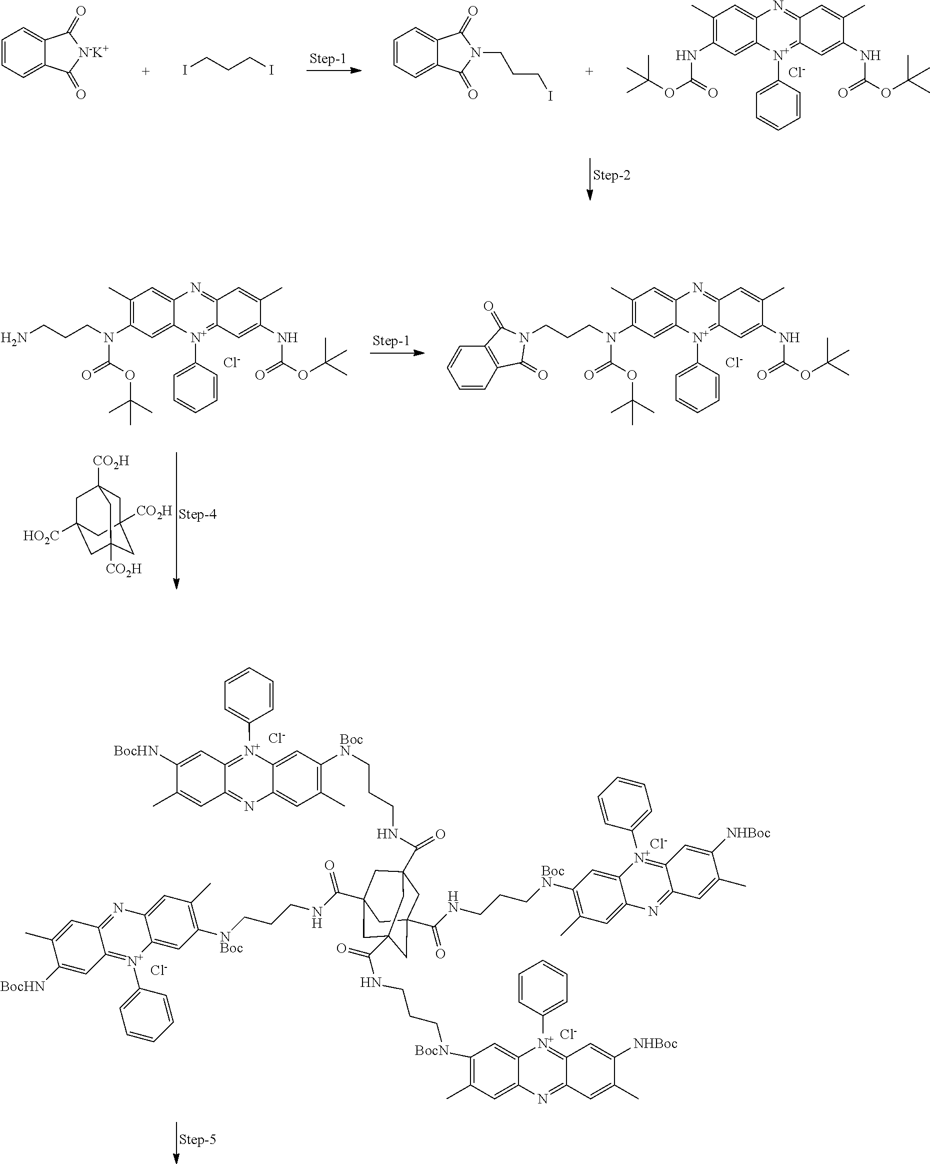

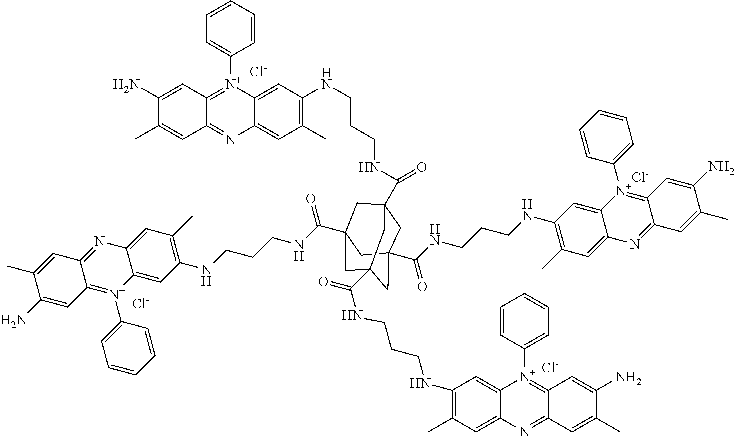

### Step-1: Synthesis of 2-(3-iodopropyl) isoindoline-1,3-dione

To a suspension of potassium 1,3-dioxoisoindolin-2-ide (3 g, 16.2 mmol) in DMF (150 mL) was added 1,3-diiodopropane (4.2 mL, 36.4 mmol) dropwise and heated at 85° C. for 2 h. The reaction mixture was monitored by LCMS. The reaction mixture was diluted with cold water and extracted with diethyl ether (2×250 mL). The combined organic layer was washed with water (3×200 mL) and brine (250 mL). The organic layer was dried over sodium sulfate and concentrated under reduced pressure. The crude product was purified by chromatography with 10% EtOAc in hexane as eluent to afford 3 g of 2-(3-iodopropyl) isoindoline-1,3-dione.

### Step-2: Synthesis of 3-(tert-butoxycarbonyl(3-(1,3-dioxoisoindolin-2-yl)propyl)amino)-7-(tert-butoxycarbonylamino)-2,8-dimethyl-5-phenylphenazin-5-ium chloride

To a solution of 3, 7-bis(tert-butoxycarbonylamino)-2,8-dimethyl-5-phenylphenazin-5-ium chloride (4 g, 7.272 mmol) in DMF (50 mL) at RT was added cesium carbonate (5.9 g, 18.1 mmol) portion-wise and stirred for 15 min. 2-(3-Iodopropyl)isoindoline-1,3-dione (2.97 g, 9.45 mol) was dissolved in DMF (10 mL) and added dropwise at 0° C. The reaction mixture was stirred at RT for 16 h. The reaction mixture was monitored by LCMS and then diluted with cold water. The precipitate was filtered, and washed with water. The precipitate was dissolved in DCM (150 mL) and dried over sodium sulfate, and concentrated under reduced pressure to give a crude product that was purified by column chromatography (neutral alumina: eluent 2% MeOH in DCM) to afford 3 g of 3-(tert-butoxycarbonyl(3-(1,3-dioxoisoindolin-2-yl)propyl)amino)-7-(tert-butoxycarbonylamino)-2,8-dimethyl-5-phenylphenazin-5-ium chloride.

### Step-3: Synthesis of 3-((3-aminopropyl)(tert-butoxycarbonyl)amino)-7-(tert-butoxycarbonylamino)-2,8-dimethyl-5-phenylphenazin-5-ium chloride

To a solution of 3-(tert-butoxycarbonyl(3-(1,3-dioxoisoindolin-2-yl)propyl)amino)-7-(tert-butoxycarbonylamino)-2,8-dimethyl-5-phenylphenazin-5-ium chloride (3 g, 4.06 mmol) in ethanol (200 mL) was added hydrazine hydrate (3.9 mL, 81.3 mmol) and heated to reflux at 85° C. for 1 h. The reaction mixture was monitored by LCMS, and then the reaction mixture was concentrated and the residue dissolved in diethyl ether and filtered. The filtrate was concentrated under reduced pressure and the crude product was purified by column chromatography (neutral alumina eluent 4% MeOH in DCM) to afford 1 g of 3-((3-aminopropyl)(tert-butoxycarbonyl)amino)-7-(tert-butoxycarbonylamino)-2,8-dimethyl-5-phenylphenazin-5-ium chloride.

### Step-4: Synthesis of BOC Protected Tetramer of Adamantane

To a solution of adamantane 1,3,5,7-tetracarboxylic acid (30 mg, 0.0961 mmol) in DMF (6 mL) were added EDC.HCl (137 mg, 0721 mmol) and HOBT (97 mg, 0.721 mmol) and the mixture stirred at RT for 15 min. Then 3-((3-aminopropyl)(tert-butoxycarbonyl)amino)-7-(tert-butoxycarbonylamino)-2,8-dimethyl-5-phenylphenazin-5-ium chloride (263 mg, 0.432 mole) was added portion-wise and the mixture stirred at RT for 16 h. The reaction mixture was monitored by LCMS, and then diluted with water. The precipitate was filtered and washed with water. The residue was dissolved in DCM (75 mL), dried over sodium sulfate and concentrated under reduced pressure. The crude product was purified by column chromatography (Neutral alumina eluent 3% MeOH in DCM) to afford 125 mg of BOC protected tetramer of adamantane.

### Step-5: Synthesis of {3-amino-2,8-dimethyl-5-phenyl-7-[(3-{[3,5,7-tris({3-[(7-amino-5-chloranuide-2,8-dimethyl-5-phenyl-5λ⁶,10-phenazin-5-ylium-3-yl)amino]propyl}carbamoyl)adamantan-1-yl]formamido}propyl)amino]-5λ⁶,10-phenazin-5-ylium-5-e}chloranuide

To the BOC protected tetramer of adamantane from Step-4 (125 mg, 0.046 mmol) in DCM (4 mL) at 0° C. was added TFA (0.5 mL) dropwise and the mixture allowed to stir at RT for 16 h. The reaction mixture was monitored by LCMS and solvent was concentrated to obtain a crude product that was purified by reverse phase HPLC to obtain desired product. This product was treated with methanolic HCl (4×20 mL), concentrated each time, then triturated with water (5 mL), filtered and washed with water (20 mL). The residue was dried by lyophilization to afford 25 mg of {3-amino-2,8-dimethyl-5-phenyl-7-[(3-{[3,5,7-tris({3-[(7-amino-5-chloranuide-2,8-dimethyl-5-phenyl-5λ⁶,10-phenazin-5-ylium-3-yl)amino]propyl}carbamoyl)adamantan-1-yl]formamido}propyl)amino]-5λ⁶,10-phenazin-5-ylium-5-e}chloranuide. ¹H NMR (DMSO): δ (ppm): 7.60-7.90 (m, 44H), 6.0 (s, 4H), 5.45 (s, 4H), 2.90-3.0 (m, 16H), 2.30 (s, 12H), 2.25 (s, 12H), 1.76 (s, 12H), 1.65-1.75 (m, 8H). LCMS: 1728.9, M/4=432.8, M/3=576.5, M/2=864.3.

### Example 16. Synthesis of Compound 26

### Synthesis of (3-amino-7-{[6-({6-[5-({6-[(7-amino-5-chloranuide-2,8-dimethyl-5-phenyl-5λ⁶,10-phenazin-5-ylium-3-yl)amino]hexyl}carbamoyl)pyridin-2-yl]pyridin-3-yl}formamido)hexyl]amino}-2,8-dimethyl-5-phenyl-5λ⁶,10-phenazin-5-ylium-5-e)chloranuide

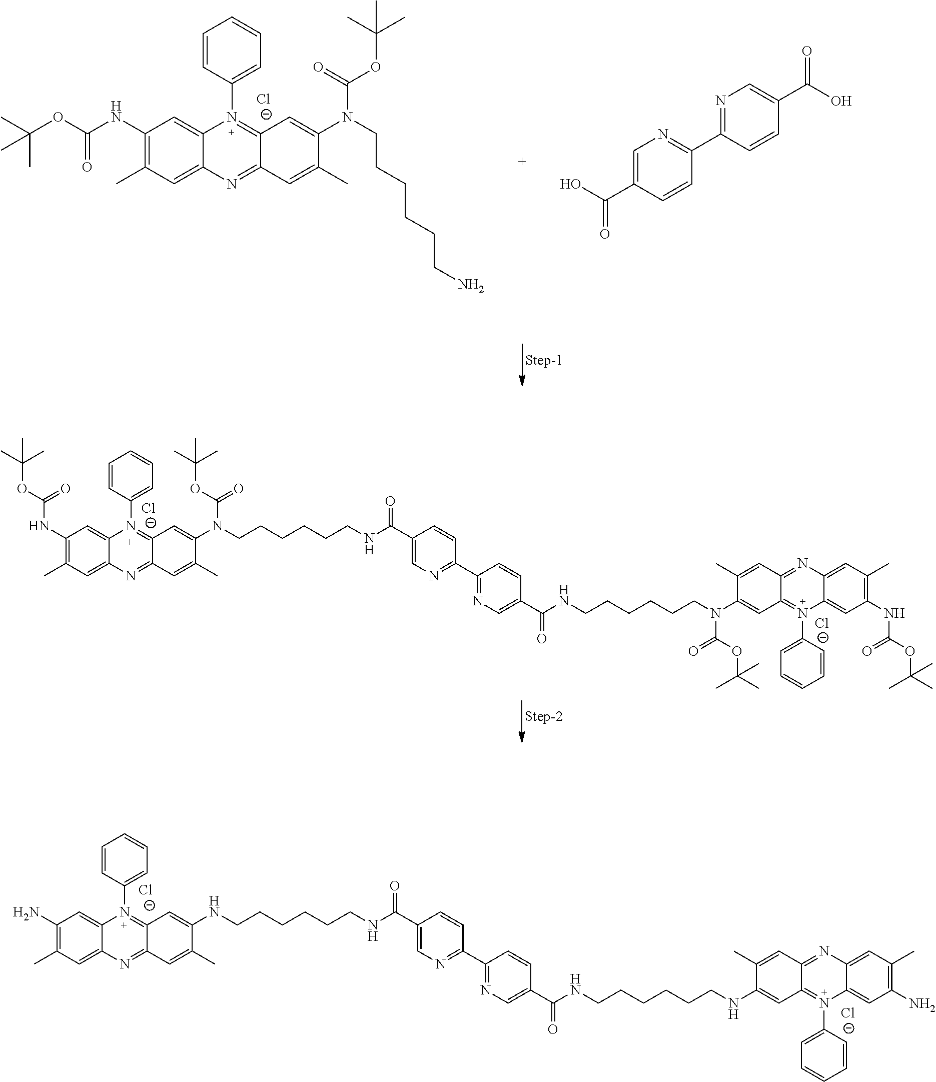

### Step-1: Synthesis of tert-butyl N-[8-(tert-butoxycarbonylamino)-3,7-dimethyl-10-phenyl-phenazin-10-ium-2-yl]-N-[6-[[6-[5-[6-[tert-butoxycarbonyl-[8-(tert-butoxycarbonylamino)-3,7-dimethyl-10-phenyl-phenazin-10-ium-2-yl]amino]hexylcarbamoyl]-2-pyridyl]pyridine-3-carbonyl]amino]hexyl]carbamate dichloride

To a suspension of 2,2′-bipyridine-5,5′-dicarboxylic acid (50 mg, 0.204 mmol) in DMF (5 mL) were added EDC.HCl (155 mg, 0.816 mmol) and HOBt (109 mg, 0.816 mmol) and the mixture stirred at RT for 20 min. Then 3-((6-aminohexyl)(tert-butoxycarbonyl)amino)-7-(tert-butoxycarbonylamino)-2,8-dimethyl-5-phenylphenazin-5-ium chloride (293 mg, 0.450 mmol) and DIPEA (0.35 mL, 2.04 mmol) were added portion-wise and the mixture stirred at RT for 12 h. The reaction mixture was monitored by LCMS, and then diluted with water, extracted with EtOAc (2×25 mL). The combined organic layer was washed with brine (4×25 mL), dried over sodium sulfate, and concentrated under reduced pressure. The crude product was purified by column chromatography (neutral alumina eluent 5% MeOH in DCM) to afford 125 mg of BOC protected dimer.

### Step-2: Synthesis of N-[6-[(8-amino-3,7-dimethyl-10-phenyl-phenazin-10-ium-2-yl)amino]hexyl]-6-[5-[6-[(8-amino-3,7-dimethyl-10-phenyl-phenazin-10-ium-2-yl)amino]hexylcarbamoyl]-2-pyridyl]pyridine-3-carboxamide dichloride

To the BOC protected dimer from Step-1 (90 mg, 0.0596 mmol) in DCM (5 mL) at 0° C. was added TFA (0.4 mL) dropwise and the mixture stirred at RT for 12 h. The reaction mixture was monitored by LCMS. The solvent was evaporated and the crude product purified by HPLC to obtain the desired product, which was treated with methanolic HCl (4×15 mL), concentrated each time, then triturated with water (5 mL), filtered and washed with water (20 mL). The residue was dried by lyophilization to afford 15 mg of title compound. 1H NMR (DMSO): δ (ppm): 9.10 (s, 2H), 8.83 (t, 2H), 8.43 (d, 2H), 8.38 (d, 2H), 7.90-7.75 (m, 10H) 7.62-7.72 (m, 2H), 7.60 (d, 4H), 7.60-7.50 (m, 2H), 6.0 (s, 2H), 5.50 (s, 2H), 3.26 (m, 4H), 2.99 (m, 4H), 2.35 (s, 6H), 2.33 (s, 6H), 1.50-1.42 (m, 4H), 1.40-1.30 (m, 4H), 1.20-1.05 (m, 8H). LCMS: 1036.56, M/3=346.3, M/2=518.9.

### Example 17. Synthesis of Compound 27

### Synthesis of {3-amino-7-[(6-{[3-({6-[(7-amino-5-chloranuide-2,8-dimethyl-5-phenyl-5λ⁶,10-phenazin-5-ylium-3-yl)amino]hexyl}carbamoyl)adamantan-1-yl]formamido}hexyl)amino]-2,8-dimethyl-5-phenyl-5λ⁶,10-phenazin-5-ylium-5-e}chloranuide

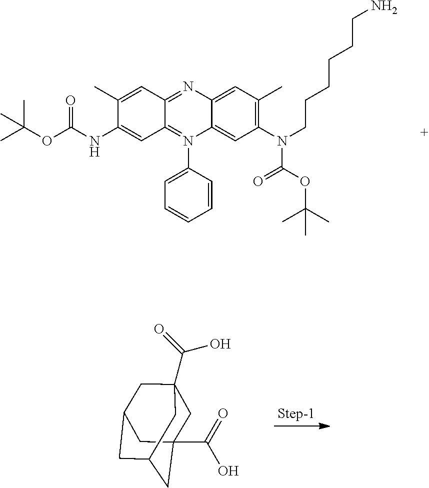

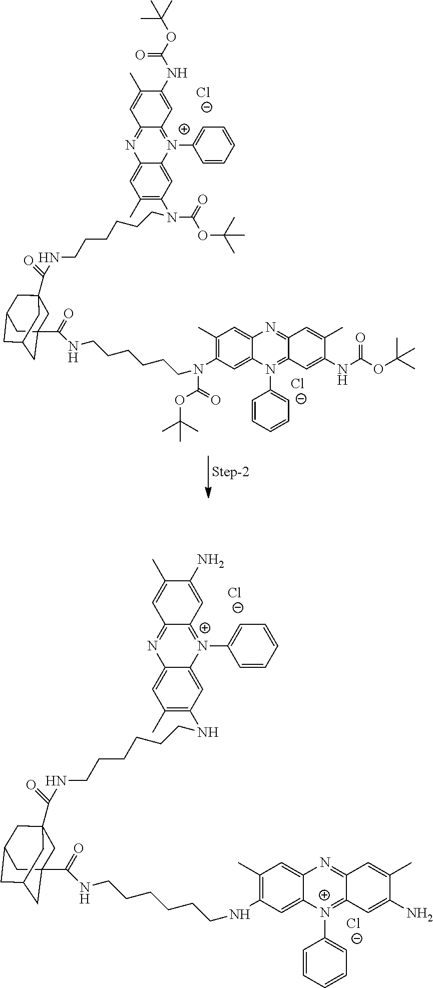

### Step-1: Synthesis of tert-butyl N-[8-(tert-butoxycarbonylamino)-3,7-dimethyl-10-phenyl-phenazin-10-ium-2-yl]-N-[6-[[3-[6-[tert-butoxycarbonyl-[8-(tert-butoxycarbonylamino)-3,7-dimethyl-10-phenyl-phenazin-10-ium-2-yl]amino]hexylcarbamoyl]adamantane-1-carbonyl]amino]hexyl]carbamate dichloride

To a solution of 1,3-adamantanedicarboxylic acid (40 mg, 0.178 mmol) in DMF (65 mL) were added EDC.HCl (119 mg, 0.623 mmol) and HOBt (84 mg, 0.623 mmol) and the mixture stirred at RT for 15 min. Then 3-((6-aminohexyl)(tert-butoxycarbonyl)amino)-7-(tert-butoxycarbonylamino)-2,8-dimethyl-5-phenylphenazin-5-ium chloride (255 mg, 0.392 mmol) and DIPEA (0.22 mL, 1.335 mmol) were added portion-wise and the mixture stirred at RT for 16 h. The reaction mixture was monitored by LCMS and diluted with water and extracted with EtOAc (2×25 mL). The combined organic layer was washed with water (2×20 mL) and brine, dried over sodium sulfate, and concentrated under reduced pressure to obtain the crude product, which was purified by column chromatography (Neutral alumina eluent 4% MeOH in DCM) to afford 150 mg of tert-butyl N-[8-(tert-butoxycarbonylamino)-3,7-dimethyl-10-phenyl-phenazin-10-ium-2-yl]-N-[6-[[3-[6-[tert-butoxycarbonyl-[8-(tert-butoxycarbonylamino)-3,7-dimethyl-10-phenyl-phenazin-10-ium-2-yl]amino]hexyl carbamoyl]adamantane-1-carbonyl]amino]hexyl]carbamate dichloride.

### Step-2: Synthesis of N1,N3-bis[6-[(8-amino-3,7-dimethyl-10-phenyl-phenazin-10-ium-2-yl)amino]hexyl]adamantane-1,3-dicarboxamide dichloride

To the compound from Step-1 (150 mg, 0.100 mmol) in DCM (3 mL) at 0° C. was added TFA (0.3 mL) dropwise and the mixture stirred at RT for 16 h. The reaction mixture was monitored by LCMS, then the solvent was concentrated and the crude product was purified by HPLC to obtain pure product. This was treated with methanolic HCl (4×15 mL), concentrated each time, then triturated with water (5 mL), filtered, and washed with water (10 mL). The residue was vacuum dried under lyophilization to afford 20 mg of N1,N3-bis[6-[(8-amino-3,7-dimethyl-10-phenyl-phenazin-10-ium-2-yl)amino]hexyl]adamantane-1,3-dicarboxamide dichloride. ¹H NMR (DMSO): δ (ppm): 7.80-7.90 (m, 10H), 7.60 (d, 6H), 7.50-7.60 (m, 2H), 7.40-7.45 (m, 2H), 6.02 (s, 2H), 5.50 (s, 2H), 2.98-3.04 (m, 8H), 2.30 (s, 6H), 2.28 (s, 6H), 2.04 (m, 2H), 1.8 (m, 2H), 1.70-1.75 (m, 8H), 1.55-1.60 (m, 2H), 1.25-1.40 (m, 10H), 1.05 (m, 6H). LCMS: 1016.6 (M), 508.6 (M/2).

### Example 18. Synthesis of Compounds 28 and 29

### Synthesis of 7,7′-(dodecane-1,12-diylbis(azanediyl))bis(3-amino-2,8-dimethyl-5-phenylphenazin-5-ium) chloride and 3-amino-7-(12-(7-amino-2,8-dimethyl-5-phenylphenazin-5-ium-3-ylamino)dodecylamino)-2,6-dimethyl-5-phenylphenazin-5-ium chloride

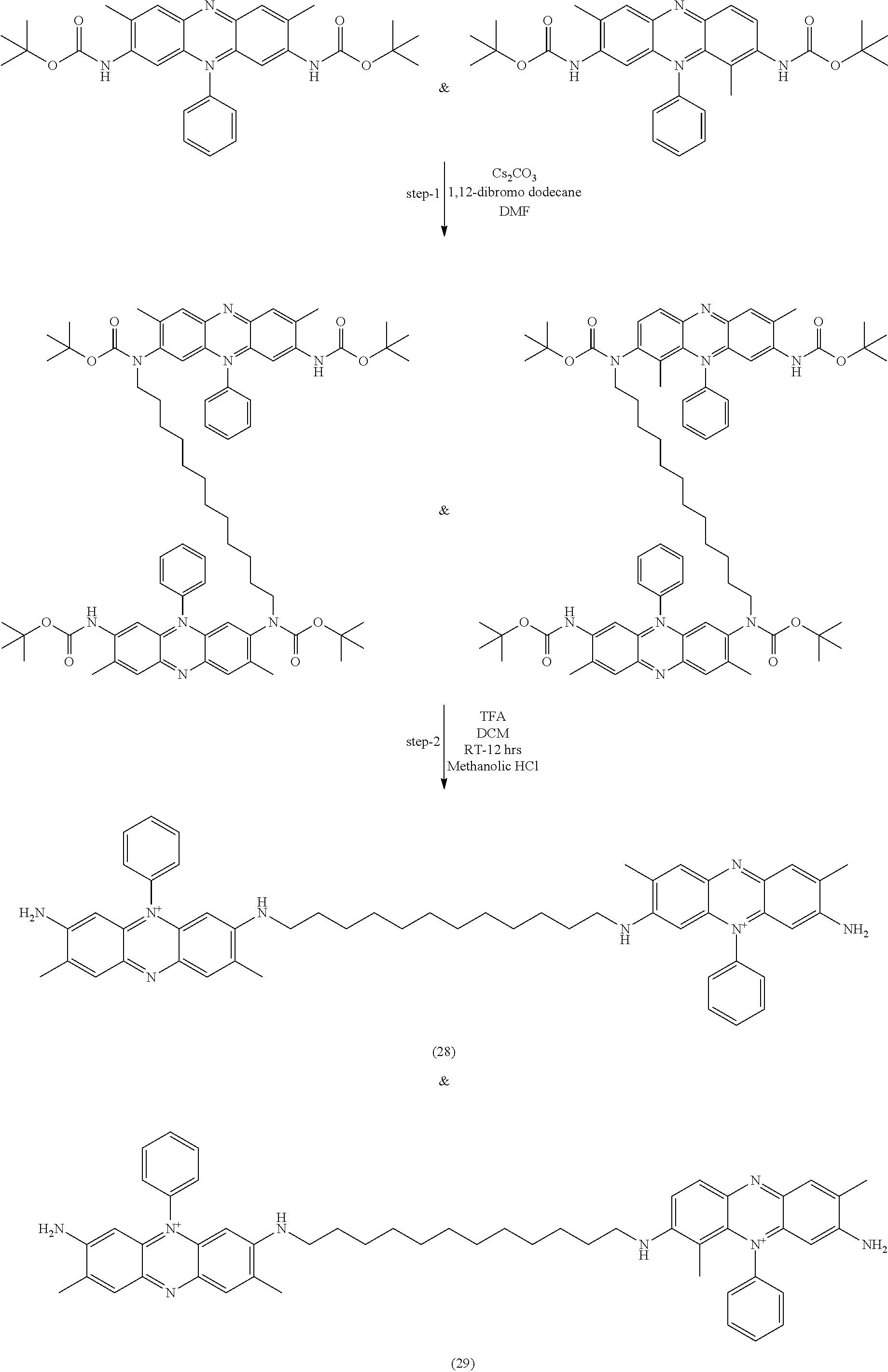

### Step-1: Synthesis of 7,7′-(2,2,21,21-tetramethyl-4,19-dioxo-3,20-dioxa-5,18-diazadocosane-5,18-diyl)bis(3-(tert-butoxycarbonylamino)-2,8-dimethyl-5-phenylphenazin-5-ium) chloride and 7-(tert-butoxycarbonyl(12-(tert-butoxycarbonyl(7-(tert-butoxycarbonylamino)-2,8-dimethyl-5-phenylphenazin-5-ium-3-yl)amino)dodecyl)amino)-3-(tert-butoxycarbonylamino)-2,6-dimethyl-5-phenylphenazin-5-ium chloride

To a stirred solution of a mixture of 3,7-bis(tert-butoxycarbonylamino)-2,8-dimethyl-5-phenylphenazin-5-ium chloride and 3,7-bis(tert-butoxycarbonylamino)-2,6-dimethyl-5-phenylphenazin-5-ium chloride (1.0 g, 1.81 mmol) in 15 mL of DMF was added cesium carbonate (1.48 g, 4.54 mmol) and the mixture stirred at RT for 15 min. The reaction mixture was then cooled to 0° C. and to it was added 1,12-dibromododecane (328 mg, 0.99 mmol, diluted with 1 mL of DMF) dropwise. The reaction mixture was then stirred at RT for 12 h. The reaction was monitored by TLC and LCMS. After completion of reaction, the mixture was diluted with ice-cold water and filtered. The solid residue obtained after filtration was dissolved in diethyl ether (200 mL) and washed with brine solution (3×50 mL). The ether layer was then dried over anhydrous sodium sulfate, and concentrated under reduced pressure to afford a crude product which was purified by column chromatography (neutral alumina, eluent 0-20% EtOAc in hexane) to afford the desired product (mixture of 2 products of same mass) as a dark brown solid (500 mg).

### Step-2: Synthesis of 7,7′-(dodecane-1,12-diylbis(azanediyl))bis(3-amino-2,8-dimethyl-5-phenylphenazin-5-ium) chloride and 3-amino-7-(12-(7-amino-2,8-dimethyl-5-phenylphenazin-5-ium-3-ylamino)dodecylamino)-2,6-dimethyl-5-phenylphenazin-5-ium chloride

To a stirred solution of mixture of 7,7′-(2,2,21,21-tetramethyl-4,19-dioxo-3,20-dioxa-5,18-diazadocosane-5,18-diyl)bis(3-(tert-butoxycarbonylamino)-2,8-dimethyl-5-phenylphenazin-5-ium) chloride and 7-(tert-butoxycarbonyl(12-(tert-butoxycarbonyl(7-(tert-butoxycarbonyl amino)-2,8-dimethyl-5-phenylphenazin-5-ium-3-yl)amino)dodecyl)amino)-3-(tert-butoxy carbonylamino)-2,6-dimethyl-5-phenylphenazin-5-ium chloride (500 mg, 0.39 mmol) in 5 mL of DCM was added TFA (2 mL) at 0° C. dropwise and stirred at RT for 12 h. The reaction was monitored by TLC and LCMS. After completion of reaction, the solvent was removed under reduced pressure to dryness to obtain a crude product which was purified by HPLC to afford two peaks of the same mass. Both peaks were then treated separately with Methanolic HCl and dried and then washed with water, and dried again to afford the desired products (Compound 28: 35 mg and Compound 29: 55 mg). Compound 28: ¹H NMR (DMSO-d6): δ (ppm): 7.90-7.78 (m, 10H), 7.75-7.70 (m, 3H), 7.65-7.55 (m, 7H), 6.02 (s, 2H), 5.58 (s, 2H), 2.98 (m, 4H), 2.35 (s, 6H), 2.33 (s, 6H), 1.40-1.30 (m, 4H), 1.25-1.20 (bs, 10H), 1.20-1.05 (m, 6H). Compound 29: ¹H NMR (DMSO-d6): δ (ppm): 7.95 (d, 1H), 7.90-7.60 (m, 15H), 7.60-7.38 (m, 3H), 6.15 (s, 1H), 6.0 (s, 1H), 5.54 (s, 1H), 3.42 (m, 2H), 2.98 (m, 2H), 2.30 (m, 9H), 1.60 (m, 2H), 1.40 (s, 3H), 1.38-1.20 (m, 14H), 1.18-1.02 (m, 4H).

### Example 19. Synthesis of Compounds 4 and 31

### Synthesis of 7,7′-(octane-1,8-diylbis(azanediyl))bis(3-amino-2,8-dimethyl-5-phenylphenazin-5-ium) chloride & 3-amino-7-(8-(7-amino-2,8-dimethyl-5-phenylphenazin-5-ium-3-ylamino) octylamino)-2,6-dimethyl-5-phenylphenazin-5-ium chloride

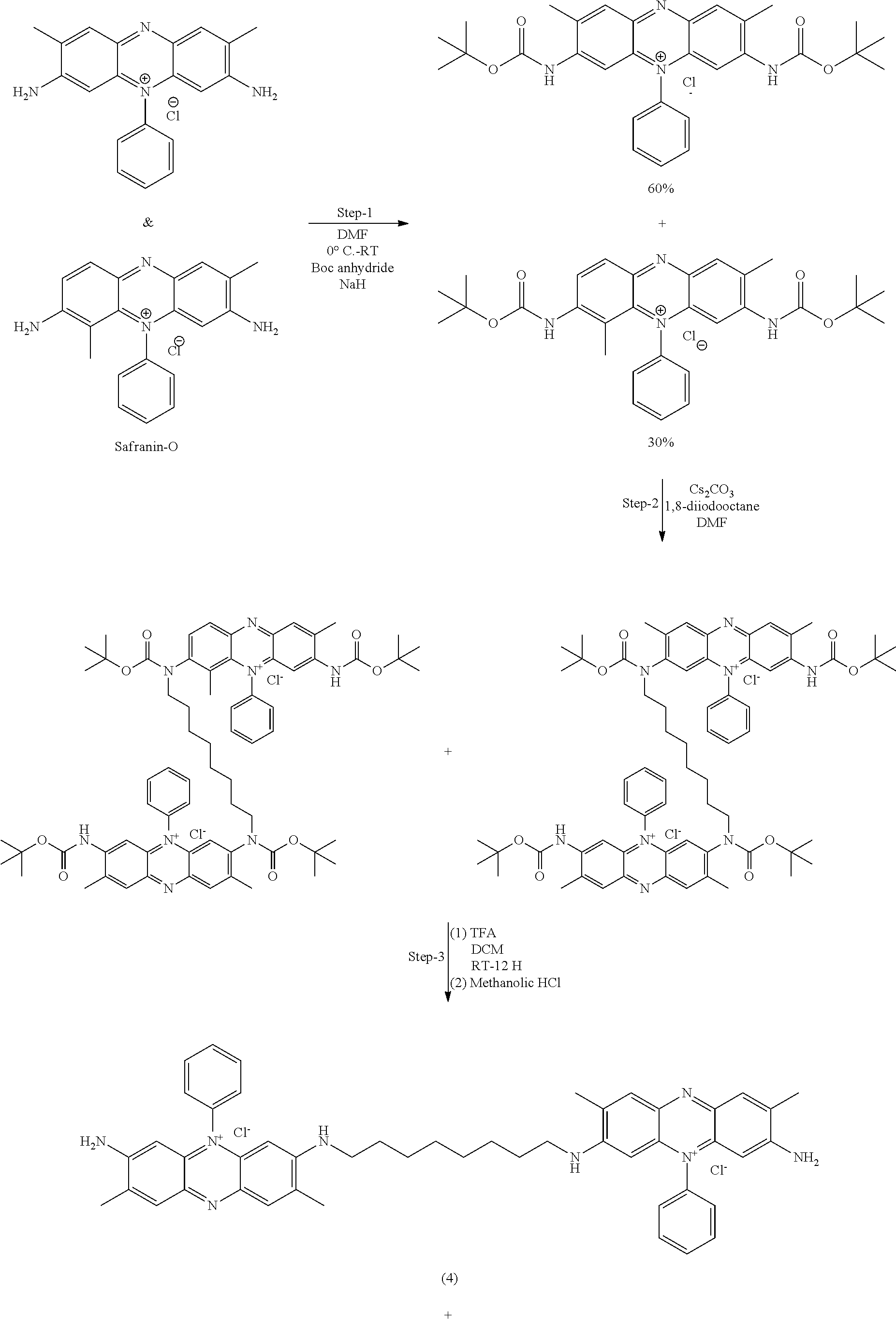

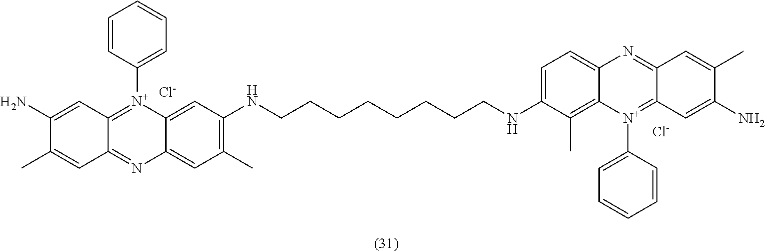 [SMILES: Cc1cc2nc3cc(C)c(NCCCCCCCCNc4ccc5nc6cc(C)c(N)cc6[n+](-c6ccccc6)c5c4C)cc3[n+](-c3ccccc3)c2cc1N ; [Cl-] ; [Cl-]]

### Step-1: Synthesis of 3,7-bis(tert-butoxycarbonylamino)-2,8-dimethyl-5-phenylphenazin-5-ium chloride and 3,7-bis(tert-butoxycarbonylamino)-2,6-dimethyl-5-phenylphenazin-5-ium chloride

To a stirred solution of Safranin-O (10.0 g, 28.57 mmol) in 100 mL of DMF was added sodium hydride (3.42 g, 85.71 mmol) portion-wise at 0° C. After addition, the reaction mixture was stirred at RT for 15 min. Boc anhydride (12.45 g, 57.14 mmol, diluted with 15 mL of DMF) was added dropwise into the reaction mixture and stirred at RT for 2 h. The reaction was monitored by TLC and LCMS. After completion of reaction, the mixture was quenched using MeOH (30 mL), and concentrated under reduced pressure to obtain the crude product which was purified by column chromatography (neutral alumina, eluent 25% DCM in hexane to 100% DCM) to afford the desired products (a mixture of two products of the same mass) as a dark brown solid (5.0 g).

### Step-2: Synthesis of 7,7′-(2,2,17,17-tetramethyl-4,15-dioxo-3,16-dioxa-5,14-diazaoctadecane-5,14-diyl)bis(3-(tert-butoxycarbonylamino)-2,8-dimethyl-5-phenylphenazin-5-ium) chloride and 7-(tert-butoxycarbonyl(8-(tert-butoxycarbonyl(7-(tert-butoxycarbonylamino)-2,8-dimethyl-5-phenylphenazin-5-ium-3-yl)amino)octyl)amino)-3-(tert-butoxycarbonylamino)-2,6-dimethyl-5-phenylphenazin-5-ium chloride

To a stirred solution of a mixture of 3,7-bis(tert-butoxycarbonylamino)-2,8-dimethyl-5-phenylphenazin-5-ium chloride and 3,7-bis(tert-butoxycarbonylamino)-2,6-dimethyl-5-phenylphenazin-5-ium chloride (5.0 g, 9.07 mmol) in 60 mL of DMF was added cesium carbonate (8.87 g, 27.22 mmol) and stirred at RT for 15 min. The reaction mixture was cooled to 0° C. and to it was added 1,8-diiodooctane (2.05 g, 5.62 mmol, diluted with 5 mL of DMF) dropwise. The reaction mixture was stirred at RT for 12 h. The reaction was monitored by TLC and LCMS. After completion of reaction, the mixture was diluted with ice-cold water and filtered. The solid residue was dissolved in diethyl ether (250 mL) and washed with brine solution (3×50 mL). The ether layer was dried over anhydrous sodium sulfate, and concentrated under reduced pressure to afford the crude product which was purified by column chromatography (neutral alumina, eluent 0-40% EtOAc in hexane) to afford the desired product (mixture of 2 products of same mass) as a dark brown solid (3 g).

### Step-3: Synthesis of 7,7′-(octane-1, 8-diylbis(azanediyl))bis(3-amino-2, 8-dimethyl-5-phenylphenazin-5-ium) chloride and 3-amino-7-(8-(7-amino-2,8-dimethyl-5-phenylphenazin-5-ium-3-ylamino)octylamino)-2,6-dimethyl-5-phenylphenazin-5-ium chloride

To a stirred solution of a mixture of 7,7′-(2,2,17,17-tetramethyl-4,15-dioxo-3,16-dioxa-5,14-diazaoctadecane-5,14-diyl)bis(3-(tert-butoxycarbonylamino)-2,8-dimethyl-5-phenylphenazin-5-ium) chloride and 7-(tert-butoxycarbonyl(8-(tert-butoxycarbonyl(7-(tert-butoxycarbonyl amino)-2,8-dimethyl-5-phenylphenazin-5-ium-3-yl)amino)octyl)amino)-3-(tert-butoxy carbonylamino)-2,6-dimethyl-5-phenylphenazin-5-ium chloride (2.5 g, 2.06 mmol) in 25 mL of DCM was added TFA (10 mL) at 0° C. dropwise and the mixture stirred at RT for 12 h. The reaction was monitored by TLC and LCMS. After completion of reaction, the solvent was removed under reduced pressure to dryness to obtain a crude product (2 g mixture of two compounds). 400 mg of crude product was purified by reverse phase HPLC to afford two peaks of the same mass. Both peaks were then treated separately with methanolic HCl and dried, and then washed with water and dried again to afford both regioisomeric products.

### Example P1. Synthesis of Compounds 2, 3, 5, and 7-17

Compounds 2, 3, 5 and 7-17 can be synthesized using the conditions provided in the Examples presented above using appropriate starting materials.

### Example B1a. Compound 4 Binds to ARH-77 Cells

ARH-77 cells (an EBV-transformed B lymphoblastoid cell line established from cells of a patient with plasma cell leukemia) were incubated as indicated with PBS or 10 μM Compound 4 for 30 min. Cells were washed twice with 1 mL PBS, and staining was detected using a 530 nm/600 nm filter set on a Molecular Imaging Gel Logic system.

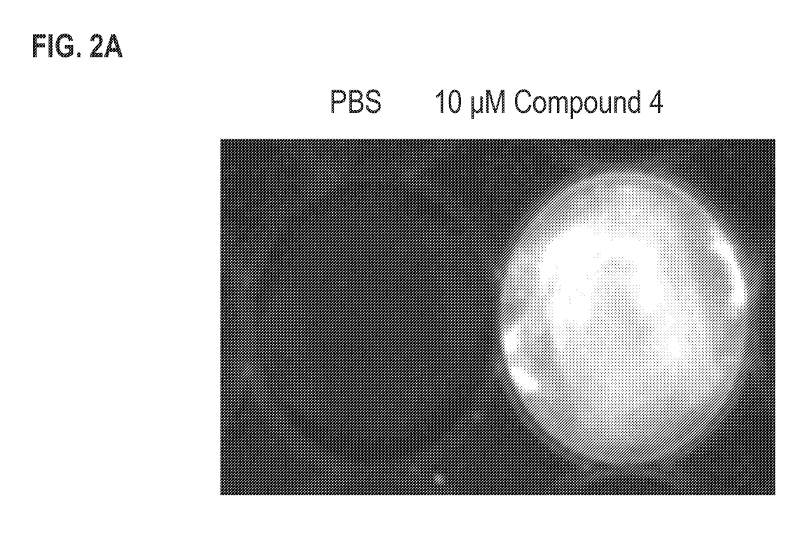

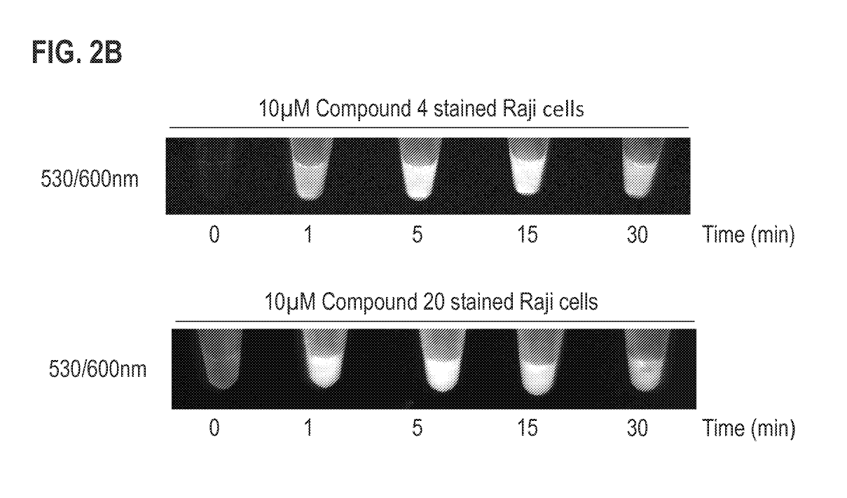

The results are shown in FIG. 2A. This example demonstrates that Compound 4 binds to ARH-77 cells.

### Example B1b. Compounds 4 and 20 Bind to Raji Cells

10⁶ Raji cells were incubated with 10 μM Compound 4 or 10 μM Compound 20 in PBS or with PBS alone for 1, 5, 15 or 30 minutes. Cells were washed once in PBS and then the fluorescence of Compound 4 or Compound 20 was analyzed in a Gel Logic system with 530 nm/600 nm excitation/emission settings.

The results are shown in FIG. 2B. This example demonstrates that Compounds 4 and 20 both bind to Raji cells.

### Example B2a. Compound 4 does not Affect Viability or Proliferation of Human MSCs at Concentrations Below 20 μM

This example reports the effects of 24 hour treatment with Compound 4 on viability and proliferation of human MSCs.

Cell proliferation was measured using the CELLTITER GLO® assay (Promega), which measures ATP levels. Cells (5×10⁵) were incubated with various concentrations of Compound 4 for 24 h. Proliferation was compared to that of untreated cells. Proliferation values for 24 h were normalized to time=0 and compared with values for untreated cells.

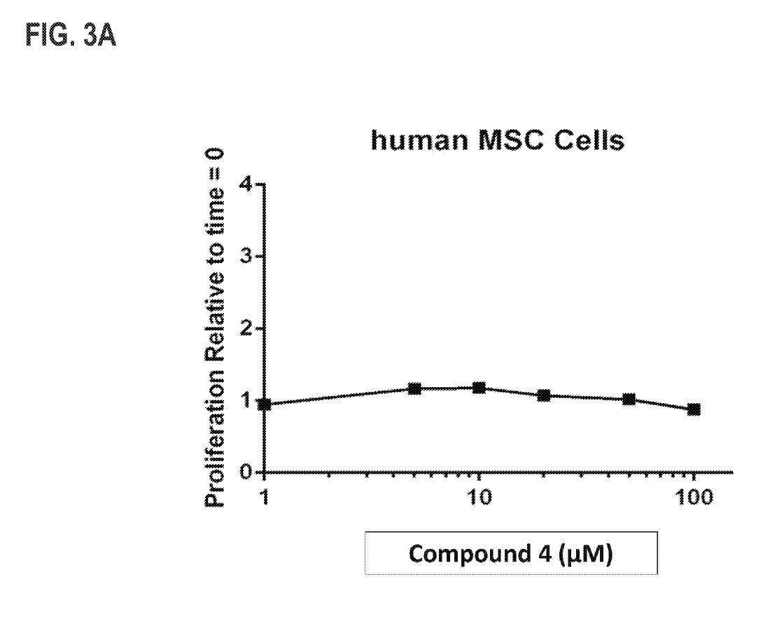

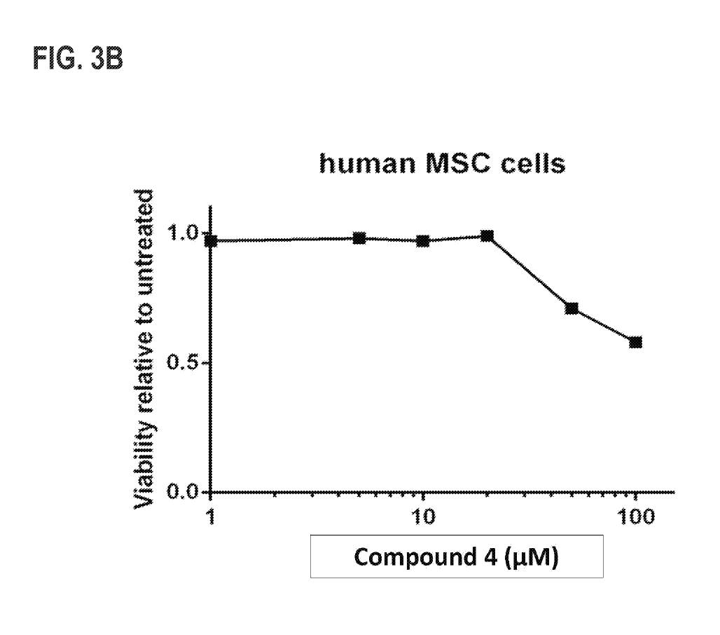

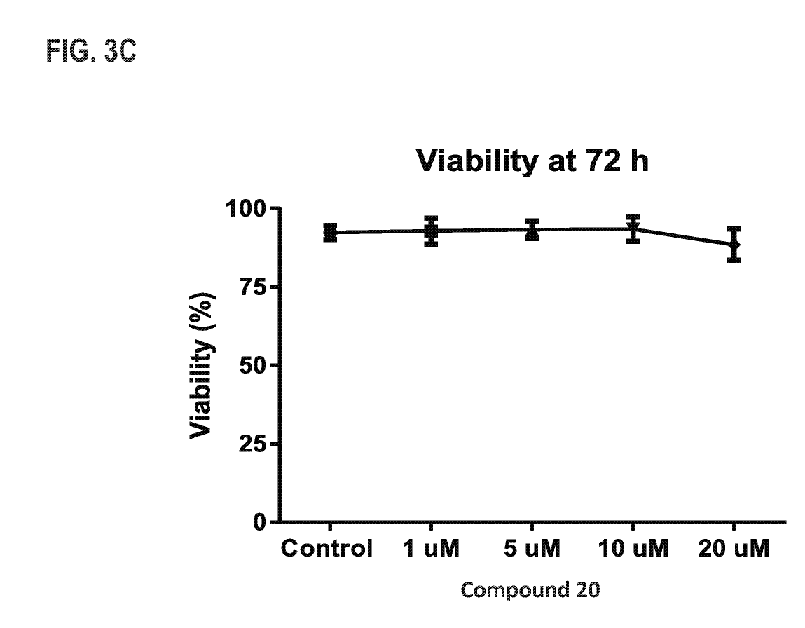

The results are shown in FIG. 3A. These results demonstrate that Compound 4 does not affect human MSC proliferation in 24 h at any of the concentrations tested.

Cell viability was measured by using trypan blue to stain dead cells. Cells (2×10⁵) were incubated with various concentrations of Compound 4 for 30 min, and viable cells were counted and compared to the number of viable cells in an untreated sample. Values for treated cells were normalized to untreated cells.

The results are shown in FIG. 3B. These results demonstrate that, at concentrations 20 μM or lower, Compound 4 did not affect the viability of human MSCs.

### Example B2b. Compound 20 does not Affect Viability of Raji Cells at Concentrations at or Below 20 μM

In a similar fashion to Example B2a, Compound 20 was assessed for its effect on viability of Raji cells as determined by trypan blue staining, as described in Example B2a.

The results are shown in FIG. 3C. Compound 20 did not have toxic effects on Raji cells within the times and concentrations tested.

### Example B3a. Compound 4 Binds to Human MSCs

Human MSCs were incubated without Compound 4 or with Compound 4 (5 μM or 10 μM) for 30 min and then washed twice with 1 mL PBS. Samples were visually inspected to determine staining

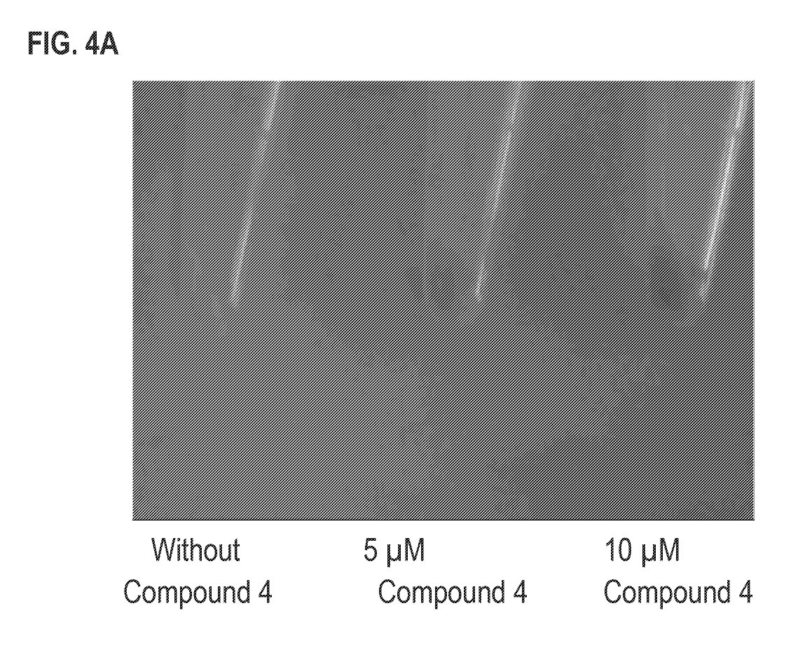

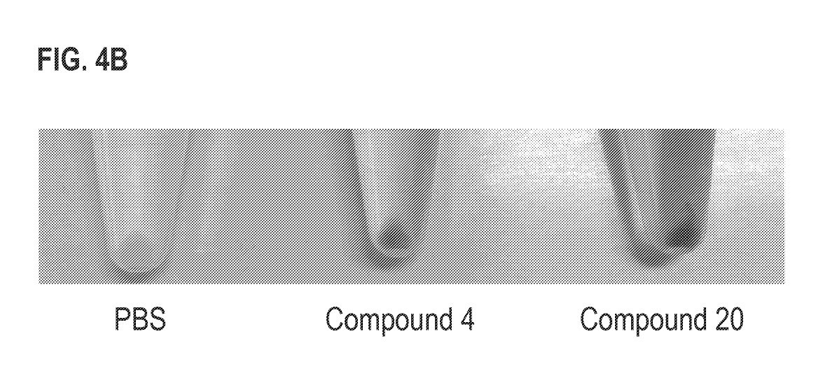

The results are shown in FIG. 4A. Compound 4 binds human MSCs both at 5 μM and 10 μM. Binding was more marked at the higher concentration.

### Example B3b. Compounds 4 and 20 Bind to Raji Cells

Raji cells were incubated in PBS without Compounds 4 or 20 or with 10 μM Compound 4 or 10 μM Compound 20 in PBS for 30 min and then washed twice with 1 mL PBS. Samples were visually inspected to determine staining

The results are shown in FIG. 4B. Compounds 4 and 20 both bind to Raji cells at 10 μM.

### Example B4. Compounds 4 and 20 Bind to Rabbit Dorsal Femoral Condyle Explants

Rabbit dorsal femoral condyle explants were incubated with PBS or 100 μM Compound 4 or Compound 20 in PBS for 30 min. Explants were washed twice in excess PBS. Staining of explants was determined using the 530/600 nm filter set on the Molecular Imaging Gel Logic system.

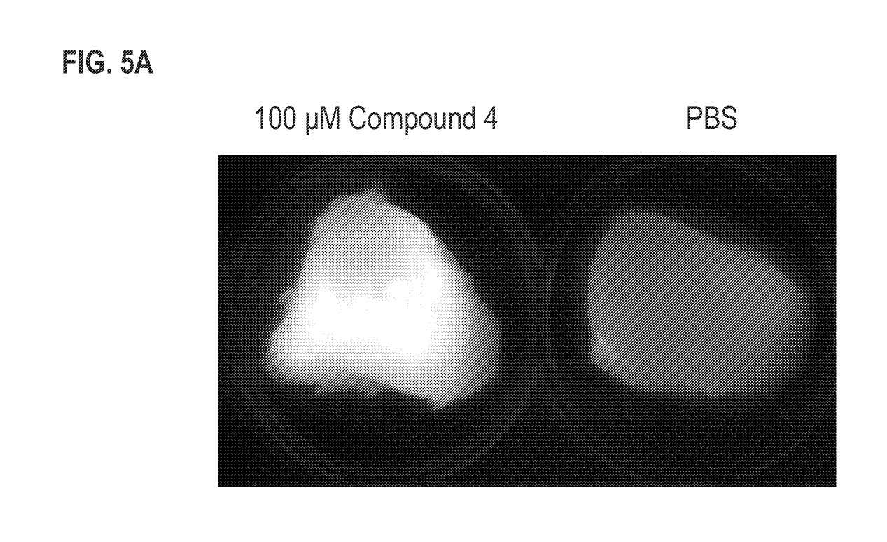

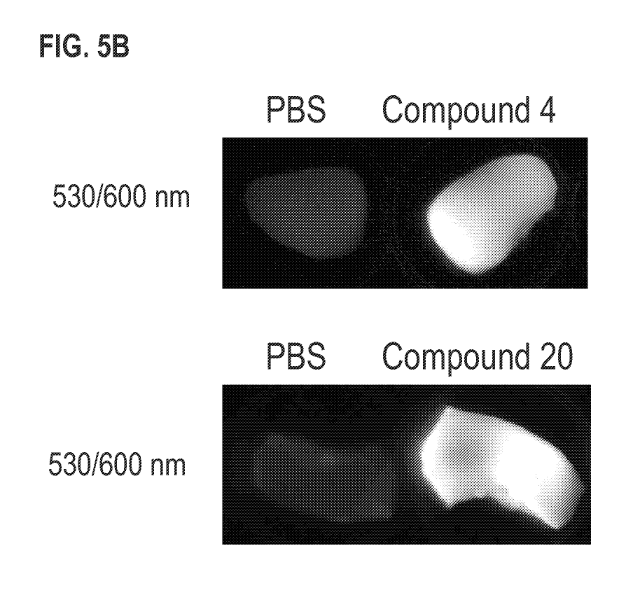

The results are shown in FIGS. 5A (Compound 4) and 5B (Compounds 4 and 20). Compounds 4 and 20 both stained rabbit dorsal femoral condyle explants as detected by the bright signal from the 600 nm emission filter. As compared to the PBS incubated sample, the explants incubated in Compound 4 or Compound 20 both showed strong fluorescence, indicating that Compounds 4 and 20 bound to the explants.

### Example B5a. Compounds 4 and 20 Promote Adherence of Human MSCs to Rabbit Dorsal Femoral Condyle Explants

This example demonstrates that Compounds 4 and 20 promote adherence of human MSCs to rabbit dorsal femoral condyle explants.

Human MSCs were labeled with calcein for 30 min, washed with PBS, and incubated with Compound 4 (5 μM or 10 μM) or with PBS for 30 min. Rabbit dorsal femoral condyle explants were then incubated for 30 min with (1) the PBS-incubated, calcein-labeled MSCs, (2) the calcein-labeled MSCs incubated with 5 μM Compound 4, or (3) the calcein-labeled MSCs incubated with 10 μM Compound 4. Explants were washed twice with excess PBS, and the 475/535 nm filter set on the Molecular Imaging Gel Logic system was used to detect the binding of MSCs to the explants by detecting the calcein. In another experiment, the procedure was repeated with two batches of Compound 4, and with Compound 20, with an incubation time of 10 min instead of 30 min.

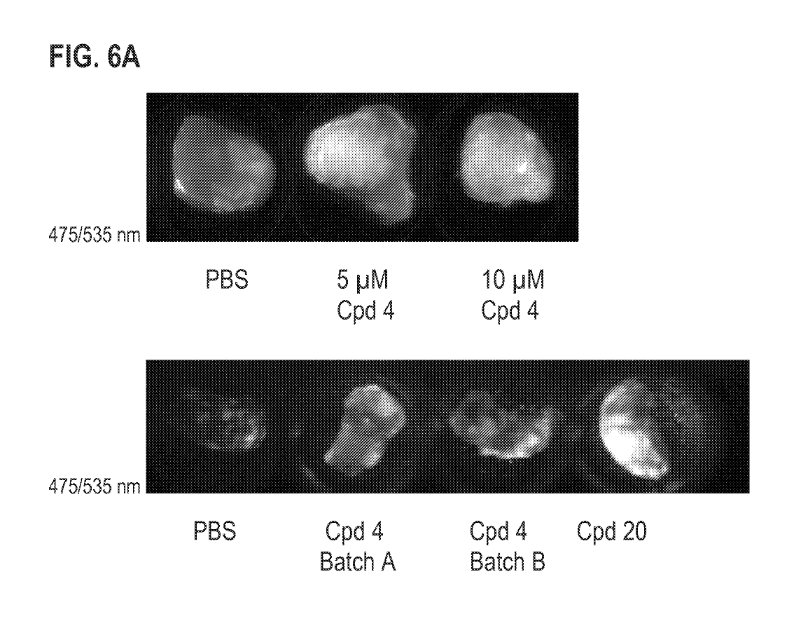

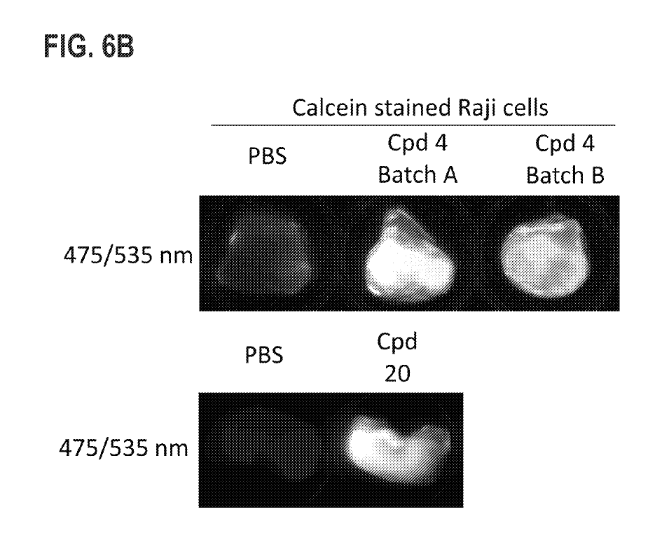

The results are shown in FIG. 6A. Explants that were incubated with calcein-labeled human MSCs that were not incubated with Compounds 4 or 20 (FIG. 6A upper left, lower left) showed basal staining in the 475/535 channel, which indicates the calcein-labeled MSCs did not strongly adhere to the explants. Explants that were incubated with calcein-labeled human MSCs incubated with Compounds 4 or 20 (FIG. 6A upper center and right, lower center and right) show increased staining in the 475/535 nm channel, which indicates that Compounds 4 and 20 promote adherence of the calcein-labeled MSCs to the explants.

### Example B5b. Compounds 4 and 20 Promote Adherence of Raji Cells to Rabbit Dorsal Femoral Condyle Explants

This example demonstrates that both Compound 4 and Compound 20 promote adherence of Raji cells to rabbit femoral condyle explants.

Raji cells were labeled with calcein for 30 min, washed with PBS, and incubated with Compound 4 or Compound 20 (10 μM in PBS) or with PBS for 30 min. Cells were washed in PBS. Rabbit dorsal femoral condyle explants were then incubated for 30 min with (1) the PBS-incubated, calcein-labeled Raji cells or (2) the calcein-labeled Raji cells that had been incubated with 10 μM Compound 4 or Compound 20. Explants were washed twice with excess PBS, and the 475/535 nm filter set on the Molecular Imaging Gel Logic system was used to detect the binding of MSCs to the explants by detecting the calcein.

The results are shown in FIG. 6B. Explants that were incubated with calcein-labeled Raji cells that were incubated with PBS but not with Compounds 4 or 20 (FIG. 6B, above left, below left) show no staining in the 475/535 channel, which indicates the calcein-labeled Raji cells did not adhere to the explants. Explants that were incubated with calcein-stained Raji cells that had been incubated with Compound 4 batch A or batch B (FIG. 6B, above center and above right) or Compounds 20 (FIG. 6B, below) did show staining in the 475/535 channel, which indicates that both Compound 4 and Compound 20 promoted adherence of the calcein-labeled Raji cells to the explants.

### Example B6. Compound 20-Incubated hMSCs Reduce Cartilage Damage in an Articular Cartilage Degeneration Model

This example demonstrates that Compound 20-incubated hMSCs reduce cartilage damage in an monosodium iodoacetate (MIA) model or articular cartilage degeneration.

6 to 8 week old Sprague Dawley rats were anesthetized with 3% sevofluorane and injected in both the right and left knee intra-articularly with MIA (monosodium iodoacetate, Sigma Cat No I2512) at a dose of 3 mg in a volume of 50 pt. MIA was dissolved in sterile saline (0.9%). hMSCs (Lonza, Cat No PT-2501) were cultured in complete MSCCM™ mesenchymal stem cell culture medium (Lonza, Cat No PT-3238).

Seven days after the injection of MIA, 2-10 million hMSCs were collected and incubated for 20 min in 500 μL of 10 μM Compound 20 in PBS or in PBS alone (untreated). Cells were washed with PBS and counted.

Rats were again anesthetized with 3% sevofluorane. 4×10⁵ hMSCs incubated in PBS or in Compound 20 were injected intraarticularly through the patellar ligament. Untreated hMSC were injected into the left knee joint, and Compound 20-stained hMSCs were injected into the right knee joint.

Twenty eight days after the injection of MIA, rats were euthanized, and femoral condyles were extracted for macroscopic evaluation.

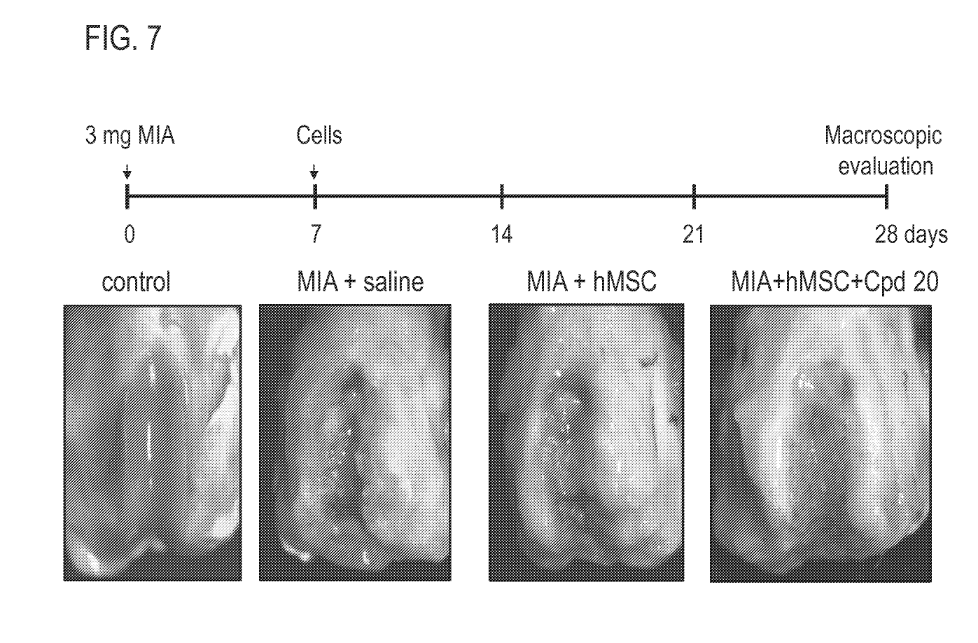

The results are shown in FIG. 7. The ‘MIA+saline’ example shows severe degradation of the cartilage and some surrounding bone material. The ‘MIA+hMSC’ example indicates a slight recovery of the cartilage area but with clear degradation remaining. The ‘MIA+hMSC+Cpd 20’ (Compound 20) example shows an improvement in the cartilage and surrounding bone area.

### Example B7. Differentiation of Human MSCs to Chondrocytes in Pellet Culture

hMSCs (Lonza, Cat No PT-2501) were cultured in complete mesenchymal stem cell culture medium (Lonza, Cat No PT-3238). For differentiation experiments, complete mesenchymal stem cell culture medium was removed, and hMSCs were either incubated for 30 min in chondrocyte differentiation medium containing TGFβ (R&D Systems, Cat No CCM005 and CCM006) (primed condition) or maintained in complete mesenchymal stem cell culture medium for 30 min.

hMSCs were collected, washed in PBS, and incubated in 500 μL of 10 μM Compound 4 or 20 in PBS for 20 min. Cells were washed in PBS and counted. 5×10⁵ cells were placed in a 15 mL tube. The cells were centrifuged at 500 g for 7 min, and either 500 μL of maintenance medium (Lonza, Cat No PT-3238) or chondrocyte differentiation medium was added gently. The medium was changed every 2 to 3 days for three weeks, maintaining the 500 μL volume.

After three weeks, the medium was removed, and cell pellets were fixed in 500 μL of a 4% paraformaldehyde (PFA) in PBS solution for 20 min at room temperature. The solution was removed, and 500 μL of a 4% PFA, 15% sucrose in PBS was added, and the mixture was left overnight. The solution was removed, and 500 μL of a 4% PFA, 30% sucrose in PBS was added, then the mixture was left overnight or until the pellet was in the bottom of the tube. The pellet culture was frozen in TISSUE-TEK® O.C.T. compound (Cat No 4583), and 5 μm cuts were made. The slices were fixed on a positive charged slide (Cell Path, Cat No MDB-0102-54A). A hydrophobic pen was used to draw a line around the cuts on the slide.

100-300 μL of blocking solution (1% BSA, 0.3% TRITON® X-100 in PBS) was added on top of the slices which were left for 1 h at RT. The blocking solution was extracted with a micropipette. Primary antibody (goat anti-aggrecan G1-IgD-G2 domain, R&D systems, Cat No AF1220) was diluted 1:20 in blocking solution, and 100-200 μL of this solution was added on top of the slices. The slides were left at 4° C. overnight in a humidified box. The primary antibody solutions were removed, and the slices were washed 3 times with 1% BSA in PBS for 5 min. The lines were redrawn around the slices with the hydrophobic pen. From the incubation with the secondary antibody and on, the samples were kept in the dark to avoid degrading the fluorophore.

The secondary antibodies were diluted 1:100 in blocking solution. 100-200 μL of the secondary antibody solution (donkey anti-goat IgG Alexa fluor 488, Invitrogen, Cat No A11055) was added to the slices. The slices were incubated for 1 h at RT. The slices were washed 3 times with 1% BSA in PBS for 5 min. The lines were redrawn around the slices with the hydrophobic pen.

Slices were stained with 100-200 μL of a 1:2000 dilution of DAPI in PBS for 5 min at RT. The DAPI was removed, and the slices were washed 3 times with PBS for 5 min, then washed a 4^{th} time with distilled water for another 5 min. The slides were left to dry. 30 μL of Fluorescent Mounting Medium was added, and then the slices were covered with a coverslip.

The results are presented in Table B7. The results show that incubation with Compound 4 or Compound 20 and differentiation medium (with “+” or without “−” priming) induced high levels of differentiation relative to the control without differentiation medium. Cells incubated with Compound 4 or Compound 20 and differentiation medium displayed levels of differentiation similar to those of cells incubated in differentiation medium alone.

TABLE B7
Differentiation of hMSCs to chondrocytes

| | Time | | | Differentiation | ECM area |
|---|---|---|---|---|---|
| Condition | (days) | Compound | Priming | Medium | (Aggrecan stain)* |
| 1 | 0 | − | − | − | 0 |
| 2 | 21 | − | − | − | 0.46 |
| 3 | 21 | − | − | + | 0.77 |
| 4 | 21 | − | + | − | 0.44 |
| 5 | 21 | − | + | + | 0.89 |
| 6 | 21 | Compound 4 (10 μM) | − | + | 0.74 |
| 7 | 21 | Compound 4 (10 μM) | + | + | 1.41 |
| 8 | 21 | Compound 20 (10 μM) | − | + | 0.74 |
| 9 | 21 | Compound 20 (10 μM) | + | + | 0.97 |

*EMC area = aggrecan ratio (relative to cell number)

### Example B8. Differentiation of Sheep MSCs to Chondrocytes in Pellet Culture

Compounds of the invention were assessed in an in vitro pellet cell culture model of chondrogenesis.

Conditions tested were:
a. (1) Priming: exposure of cells to TGFβ3 prior to incubation with Compound 4 or Compound 20;
b. (2) Dose: Concentration of Compound 4 or Compound 20 that cells were incubated with prior to pellet culture; and
c. (3) Media: presence or absence of TGFβ3 in the media used for the 3-week pellet culture duration.

A population of cells enriched for MSCs from sheep bone marrow was harvested, cultured and expanded. Cells from passage 3 (P3) were incubated with either chondrogenic differentiation medium for the Priming “+” groups or chondrogenic differentiation medium without TGFβ3 for the Priming “−” groups. Following centrifugation at 400×g for 5 min and gentle removal of the supernatant, cells were re-suspended with the appropriate number of cells in Compound 4 or Compound 20 in 1×PBS, pH 7, at following concentrations: 10 μM, 3.16 μM, 1 μM, and 0.316 μM of Compound 4; or 10 μM and 1 μM of Compound 20. For control conditions, cells were incubated in 1×PBS at pH 7. All treatment groups were then incubated for 30 min in the dark with nutation. Following centrifugation at 400×g for 5 min and gentle removal of the supernatant, cells were re-suspended and washed with 1×PBS at pH 7 and spun at 400×g for 5 min to remove supernatant. Depending on the condition, cells were re-suspended cells in chondrogenic differentiation medium OR in chondrogenic medium without TGFb3. Cells were counted, and approximately 2.5×10⁵ cells were plated into polypropylene 96-well plates incubated under standard pellet culture conditions for three weeks. Medium was changed 3× weekly.

Pellets were fixed in 10% formalin for at least 8 hours and then infiltrated with paraffin wax. Sections were then cut at approximately 5 microns and stained using safranin-orange, Fast Green, Haematoxylin-Eosin, and 1% Toluidine Blue.

Histological grading was performed according to a modified Bern Score to assess chondrogenic morphology of the cells and any matrix deposition by the cells according to the following three criteria:

| Criteria | Description | Score |
|---|---|---|
| Matrix Stain | Degree of Safranin-O stain present in the | 0 = no stain |
| | pellet representing concentration of | 3 = strong stain |
| | glycosaminoglycan | |
| Cell Spacing | Degree of separation of cells representing | 0 = no |
| | matrix deposition | separation |
| | | 3 = normal |
| | | spacing |
| Cell | Degree of chondrocyte cell morphology by | 0 = no lacunae |
| morphology | assessment of cell shape, cytoplasmic | 3 = normal |
| | space (lacunae), and nucleus shape | morphology |

The subcomponents of the Bern Score are additive. Accordingly, the Bern Score presents a maximum total score of 9 representing the best possible score and minimum score of 0 representing no chondrogenesis.

In this Example, the Bern Score for Control pellets is a proxy for chondrogenic capacity of the cell population using a standard pellet culture protocol. The Bern Score of the pellets from each treatment condition suggests the chondrogenic capacity of that cell population. Pellets that were formed using the 30 minute incubation protocol with only PBS replacing Compound 4 (0 μM Compound 4) are referred to as “PBS pellets.”

The cell population used in this study presented very good chondrogenic capacity with Bern Scores of 7.5 and 8.5 for the non-primed and primed conditions respectively. Bern Scores for each treatment are presented in Table B8. Cells incubated in Compound 4 at 3.16 μM and differentiation medium showed good chondrogenic capacity. Cells incubated in either concentration of Compound 20 tested and differentiation medium also showed good chondrogenic capacity. Priming cells with TGFβconsistently improved the Bern scores of the pellets in any of the conditions.

TABLE B8
Bern Scores.

| Differen- | | | | | Cell | Mor- | |
|---|---|---|---|---|---|---|---|
| tiation | TGFβ3 | Com- | Conc. | Matrix | Dis- | phol- | Final |
| Medium | Priming | pound | (μM) | Stain | tance | ogy | Score |
| − | − | 4 | 0 | 0 | 0 | 0 | 0 |
| − | + | 4 | 0 | 0 | 0 | 0 | 0 |
| − | + | 4 | 10 | 0 | 0 | 0 | 0 |
| − | + | 4 | 3.16 | 0.5 | 0 | 0 | 0.5 |
| − | + | 4 | 1 | 0 | 0 | 0 | 0 |
| − | + | 4 | 0.316 | 0 | 0 | 0 | 0 |
| − | + | 20 | 10 | 0 | 0 | 0 | 0 |
| − | + | 20 | 1 | 0 | 0 | 0 | 0 |
| + | − | 4 | 0 | 3 | 2.5 | 2 | 7.5 |
| + | − | 4 | 10 | 0.5 | 0 | 0 | 0.5 |
| + | − | 4 | 3.16 | 2 | 2 | 2 | 6 |
| + | − | 4 | 1 | 2.5 | 2 | 2 | 6.5 |
| + | − | 4 | 0.316 | 3 | 2.5 | 2.5 | 8 |
| + | − | 20 | 10 | 2 | 2 | 2 | 6 |
| + | − | 20 | 1 | 2.5 | 2.5 | 2 | 7 |
| + | + | 4 | 0 | 3 | 3 | 2.5 | 8.5 |
| + | + | 4 | 10 | 0 | 0 | 0 | 0 |
| + | + | 4 | 3.16 | 2 | 2 | 2 | 6 |
| + | + | 4 | 1 | 3 | 2.5 | 2 | 7.5 |
| + | + | 4 | 0.316 | 3 | 3 | 3 | 9 |
| + | + | 20 | 10 | 2.5 | 2.5 | 2.5 | 7.5 |
| + | + | 20 | 1 | 3 | 3 | 2.5 | 8.5 |

### Example B9. In Vitro and In Vivo Evaluation of Compound 4-Augmented MSCs for Cartilage Repair

The capacity of ovine MSCs derived from bone marrow and augmented with Compound 4 to bind to lesions in cartilage in vivo was evaluated in a chondral defect model.

Bone marrow aspirate was collected from fifteen healthy nave female sheep and processed in vitro to attain a population of mesenchymal stem cells (MSCs) for each sheep created by expansion of adherent cells. MSCs were frozen at passage 1 (P1).

Two circular partial thickness chondral defects were surgically created bilaterally in the medial femoral condyle of each sheep, thereby creating four defects per sheep. Once the medial femoral condyle was surgically exposed, two partial thickness circular defects were created in the cartilage of the anterior aspect, separated by 2-3 mm. Each defect was created by penetrating the cartilage with a 7 mm diameter biopsy punch to delineate the margins followed by removal of cartilage within the defect margin with a scalpel to a depth of approximately 0.5 mm-0.8 mm without penetrating the surface of the calcified cartilage. Within the same animal, each defect received one of four experimental treatments:
a. D1: Empty Control: defect left unfilled;
b. D2: Vehicle Control: cells incubated in PBS delivered to the defect;
c. D3: 100 μM Compound 4 incubated in defect followed by delivery of cells to the defect; and
d. D4: Cells were incubated with 10 μM Compound 4 for 30 min and together they were delivered to defect.

Sheep were divided into three groups of 5 sheep. One group was sacrificed on day 1 after surgery, the second group was sacrificed on day 7, and the third group was sacrificed on day 14 after surgery.

The complete intact joint was harvested and transported to the laboratory. The joint was exposed, and defects were scored macroscopically prior to separating out bone blocks containing the defects with a band saw. Defects were further trimmed and individually separated using a fine ISOMET® saw prior to fixation in 10% neutral buffered formalin. Tissue was further trimmed for histological processing, fixed, and decalcified for at least one week.

As each sample completed decalcification, it was transferred to 40% EtOH until all samples were decalcified and the last sample had been in 40% EtOH for at least 24 hours.

Following decalcification, specimens were prepared for histology staining with safranin-orange, fast green and haematoxylin-eosin.

Histological Assessment of Compound 4-Augmented Cell Retention

Histological slides were stained with Haematoxylin & Eosin and Safranin Orange for evaluation of cell retention and the early stages of cartilage repair. Cell retention was assessed using the following scoring system:

| Description | Score |
|---|---|
| No evidence of implant cells adhered to the surface | 0 |
| Evidence of implant cells adhered/retained within | 1 |
| protected regions of the defect (such as crevices) | |
| Evidence of implant cells adhered to exposed | 2 |
| surfaces of the cartilage defect | |

The results of the assessment are presented in Table B9. The data indicate that Compound 4 promotes MSC retention.

TABLE B9
Cell Retention by Treatment. (Average scores, n = 5)

| Day | D1 | D2 | D3 | D4 |
|---|---|---|---|---|
| 1 | 0 | 0 | 0.75 | 0.4 |
| 7 | 0 | 0.2 | 0.75 | 1.0 |
| 14 | 0 | 0.2 | 1.2 | 1.25 |

D1: Empty control: defect left unfilled;
D2: Vehicle control: PBS plus cells delivered to the defect;
D3: 100 μM Compound 4 incubated defect followed by delivery of cells;
D4: Cells pre-incubated with 10 μM Compound 4 for 30 min, then delivered to defect.

1. A conjugate comprising:
(a) a cationic dye moiety bound to one or more therapeutic agents, wherein the one or more therapeutic agents independently are selected from the group consisting of a nucleic acid, a protein, and a cell;
(b) a cationic dye moiety bound to one or more therapeutic agents, wherein the one or more therapeutic agents independently are selected from the group consisting of a nucleic acid, a protein, and a cell, and wherein the cationic dye moiety is selected from the group consisting of safranin-O, toluidine blue, azure A, azure B, azure C, acridine orange, acriflavine, and methylene blue; or
(c) safranin-O bound to one or more therapeutic agents independently selected from the group consisting of a small molecule, a nucleic acid, a protein, and a cell.

2-3. (canceled)

4. The conjugate of claim 1, wherein;
(a) the cationic dye moiety is present in a cationic dye multimer comprising a first cationic dye moiety, a second cationic dye moiety, and a first linker connecting the first and second cationic dye moieties,
(b) the cationic dye multimer further comprises a second linker and a third cationic dye moiety, or
(c) the cationic dye multimer further comprises a third linker and a fourth cationic dye moiety.

5-6. (canceled)

7. The conjugate of claim 4, wherein;
(a) the first and second cationic dye moieties are different cationic dye moieties;
(b) the first and second cationic dye moieties are the same cationic dye moiety;
(c) each cationic dye moiety is independently selected from the group consisting of safranin-O, toluidine blue, azure A, azure B, azure C, acridine orange, acriflavine, and methylene blue; or
(d) at least one cationic dye moiety is safranin-O and the pendant phenyl ring of the safranin-O optionally is substituted with 1-3 substituents independently selected from —NH₂, —NHR, —NR₂, —OH, —O⁻, —NHCOCH₃, —NHCOR, —OCH₃, —OR, —C₂H₅, —R, —C₆H₅, —NO₂, —NR₃⁺, halo, trihalide, —CN, —SO₃H, —COOH, —COOR, —CHO, and —COR, wherein R is C1-C6 linear or branched alkyl.

8-10. (canceled)

11. A cationic dye multimer comprising a first cationic dye moiety, a second cationic dye moiety, and a first linker connecting the first and second cationic dye moieties, wherein the first and second cationic dye moieties are independently selected from the group consisting of safranin-O, toluidine blue, azure A, azure B, and azure C.

12. The cationic dye multimer of claim 11:
(a) further comprising a second linker and a third cationic dye moiety,
(b) wherein the first and second cationic dye moieties are different cationic dye moieties,
(c) wherein the first and second cationic dye moieties are the same cationic dye moiety;
(d) wherein each cationic dye moiety is independently selected from the group consisting of safranin-O, toluidine blue, azure A, azure B, azure C, acridine orange, acriflavine, and methylene blue;
(e) wherein at least one cationic dye moiety is safranin-O and the pendant phenyl ring of the safranin-O optionally is substituted with 1-3 substituents independently selected from —NH₂, —NHR, —NR₂, —OH, —O⁻, —NHCOCH₃, —NHCOR, —OCH₃, —OR, —C₂H₅, —R, —C₆H₅, —NO₂, —NR₃⁺, halo, trihalide, —CN, —SO₃H, —COOH, —COOR, —CHO, and —COR, wherein R is C1-C6 linear or branched alkyl, or
(f) wherein the first cationic dye moiety is azure A and the second cationic dye multimer is safranin-O.

13-18. (canceled)

19. The cationic dye multimer of claim 11, comprising a linker selected from the group consisting of: 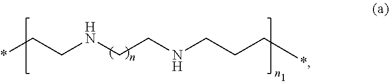
in which n is 1-6, n₁ is 1-4, and each * is an attachment site for a cationic dye moiety; 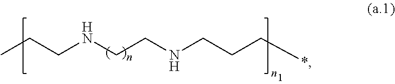
in which n is 1-6, n₁ is 1-4, and * is an attachment site for a cationic dye moiety; 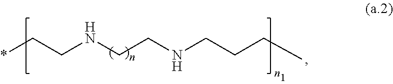
in which n is 1-6, n₁ is 1-4, and * is an attachment site for a cationic dye moiety; 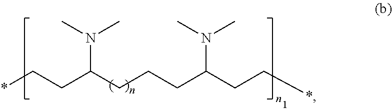
in which n is 0-6, n₁ is 1-4, and each * is an attachment site for a cationic dye moiety; 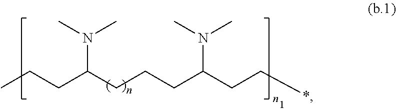
in which n is 0-6, n₁ is 1-4, and * is an attachment site for a cationic dye moiety; 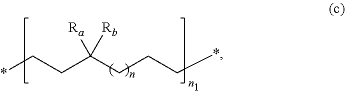
in which n is 0-6; n₁ is 1-4; for each independent instance of Rₐ and R_{b}, (1) Rₐ and R_{b} independently are H or CH₃, or (2) Rₐ and R_{b} are 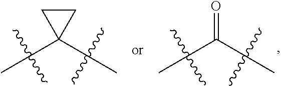
or (3) two of CRₐR_{b} are 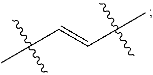 [SMILES: */C=C/*]
and each * is an attachment site for a cationic dye moiety; 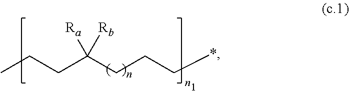
in which n is 0-6; n₁ is 1-4; for each independent instance of Rₐ and R_{b}, (1) Rₐ and R_{b} independently are H or CH₃ or (2) Rₐ and R_{b} are 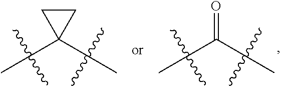
or (3) two of CRₐR_{b} are 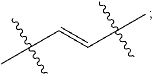 [SMILES: */C=C/*]
and * is an attachment site for a cationic dye moiety; 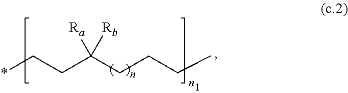
in which n is 0-6; n₁ is 1-4; for each independent instance of Rₐ and R_{b}, (1) Rₐ and R_{b} independently are H or CH₃ or (2) Rₐ and R_{b} are 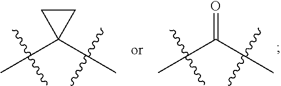
or (3) two of CRₐR_{b} are 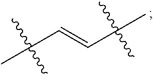 [SMILES: */C=C/*]
and * is an attachment site for a cationic dye moiety; 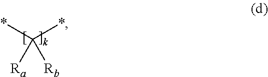
where k is 2-10; for each independent instance of Rₐ and R_{b}, Rₐ and R_{b} (1) independently are H or CH₃, or (2) Rₐ and R_{b} are 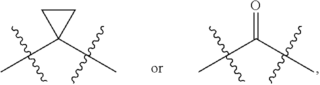
or (3) two of CRₐR_{b} are 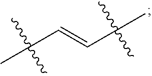 [SMILES: */C=C/*]
and each * is an attachment site for a cationic dye moiety; 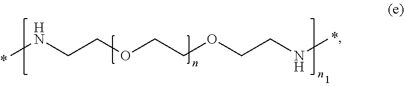
in which n is 0-6, n₁ is 1-4, and each * is an attachment site for a cationic dye moiety; 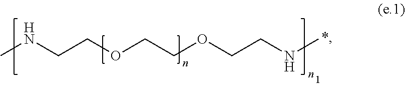
in which n is 0-6, n₁ is 1-4, and * is an attachment site for a cationic dye moiety; 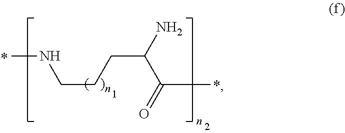
in which n₁ is 0-5, n₂ is 1-5 and each * is an attachment site for a cationic dye moiety; 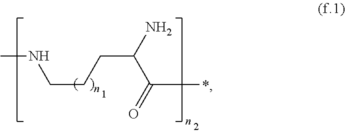
in which n₁ is 0-5, n₂ is 1-5 and * is an attachment site for a cationic dye moiety; 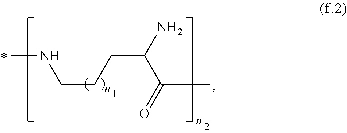
in which n₁ is 0-5, n₂ is 1-5 and * is an attachment site for a cationic dye moiety; 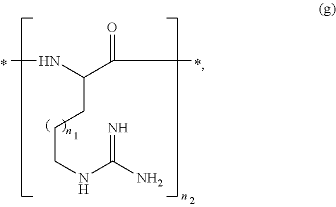
in which n₁ is 0-5, n₂ is 1-5 and each * is an attachment site for a cationic dye moiety; 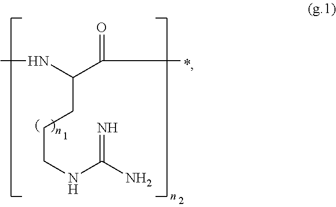
in which n₁ is 0-5, n₂ is 1-5 and * is an attachment site for a cationic dye moiety; 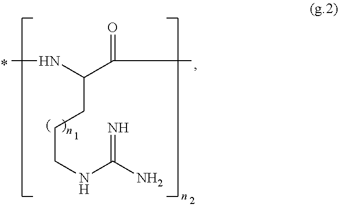
in which n₁ is 0-5, n₂ is 1-5 and * is an attachment site for a cationic dye moiety; 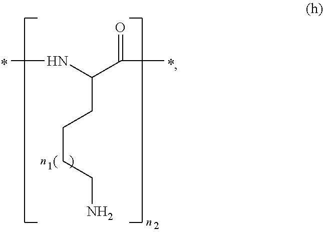
in which n₁ is 0-5, n₂ is 1-5, and each * is an attachment site for a cationic dye moiety; 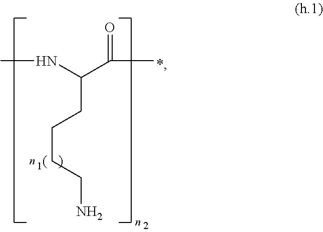
in which n₁ is 0-5, n₂ is 1-5, and * is an attachment site for a cationic dye moiety; 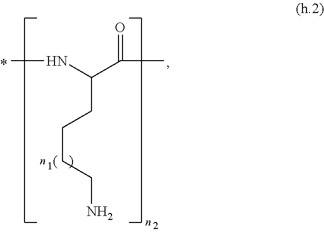
in which n₁ is 0-5, n₂ is 1-5, and * is an attachment site for a cationic dye moiety; 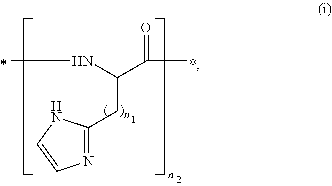
in which n₁ and n₂ independently are 1-5 and each * is an attachment site for a cationic dye moiety; 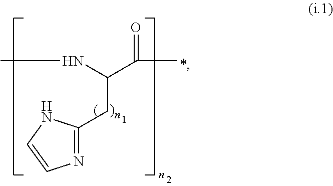
in which n₁ and n₂ independently are 1-5 and * is an attachment site for a cationic dye moiety; 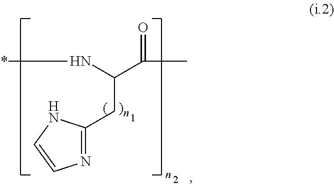
in which n₁ and n₂ independently are 1-5 and * is an attachment site for a cationic dye moiety; 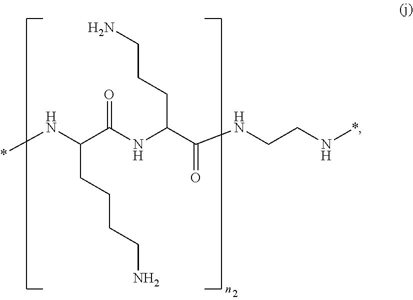
in which n₂ is 1-5 and each * is an attachment site for a cationic dye moiety; 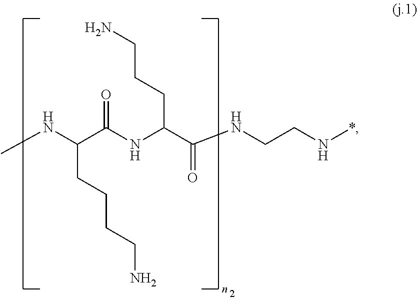
in which n₂ is 1-5 and * is an attachment site for a cationic dye moiety; 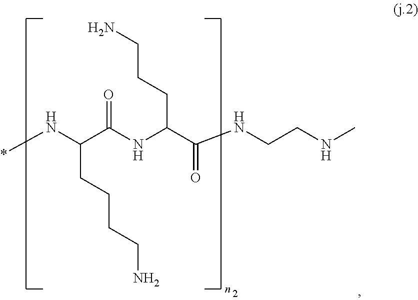
in which n₂ is 1-5 and * is an attachment site for a cationic dye moiety; 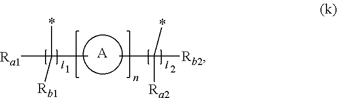
in which l₁ and l₂ independently are 1-4, n is 1-4; ring A is aryl, heteroaryl, cycloalkyl, or heterocyclyl, optionally substituted with halo or C1-C6 linear or branched alkyl; for each independent instance of Rₐ₁ and R_{b1}, Rₐ₁ and R_{b1} (1) independently are H or CH₃, or (2) Rₐ₁ and R_{b1} independently are 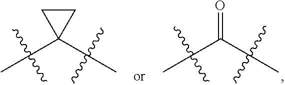
or (3) two of CRₐ₁R_{b1} are 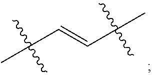 [SMILES: */C=C/*]
for each independent instance of Rₐ₂ and R_{b2}, Rₐ₂ and R_{b2} (1) independently are H or CH₃, or (2) Rₐ₂ and R_{b2} independently are 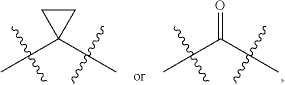
or (3) two of CRₐ₂R_{b2} are 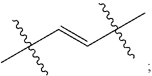 [SMILES: */C=C/*]
and each * is an attachment site for a cationic dye moiety; 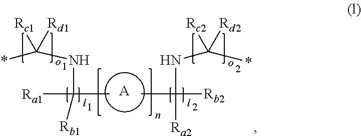
in which l₁, l₂, n, o₁, and o₂ independently are 1-4; ring A is aryl, heteroaryl, cycloalkyl, or heterocyclyl, optionally substituted with halo or C1-C6 linear or branched alkyl; for each independent instance of Rₐ₁ and R_{b1}, Rₐ₁ and R_{b1} (1) independently are H or CH₃, or (2) Rₐ₁ and R_{b1} independently are 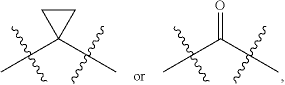
or (3) two of CRₐ₁R_{b1} are 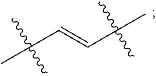 [SMILES: */C=C/*]
for each independent instance of Rₐ₂ and R_{b2}, Rₐ₂ and R_{b2} (1) independently are H or CH₃, or (2) Rₐ₂ and R_{b2} independently are 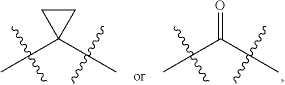
or (3) two of CRₐ₂R_{b2} are 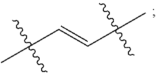 [SMILES: */C=C/*]
for each independent instance of R_{c1} and R_{d1}, R_{c1} and R_{d1} (1) independently are H or CH₃, or (2) R_{c1} and R_{d1} independently are 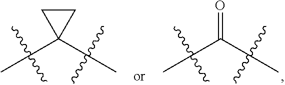
or (3) two of CR_{c1}R_{d1} are 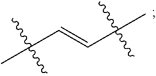 [SMILES: */C=C/*]
for each independent instance of R_{c2} and R_{d2}, R_{c2} and R_{d2} (1) independently are H or CH₃, or (2) R_{c2} and R_{d2} independently are 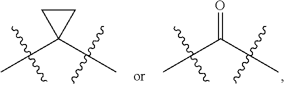
or (3) two of CR_{c2}R_{d2} are 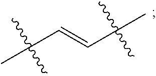 [SMILES: */C=C/*]
and each * is an attachment site for a cationic dye moiety; 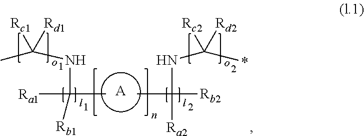
in which l₁, l₂, n, o₁, and o₂ independently are 1-4; ring A is aryl, heteroaryl, cycloalkyl, or heterocyclyl, optionally substituted with halo or C1-C6 linear or branched alkyl; for each independent instance of Rₐ₁ and R_{b1}, Rₐ₁ and R_{b1} (1) independently are H or CH₃, or (2) Rₐ₁ and R_{b1} independently are 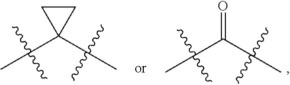
or (3) two of CRₐ₁R_{b1} are 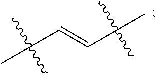 [SMILES: */C=C/*]
for each independent instance of Rₐ₂ and R_{b2}, Rₐ₂ and R_{b2} (1) independently are H or CH₃, or (2) Rₐ₂ and R_{b2} independently are 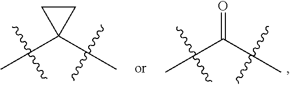
or (3) two of CRₐ₂R_{b2} are 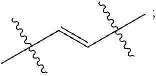 [SMILES: */C=C/*]
for each independent instance of R_{c1} and R_{d1}, R_{c1} and R_{d1} (1) independently are H or CH₃, or (2) R_{c1} and R_{d1} independently are 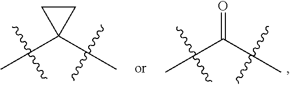
or (3) two of CR_{c1}R_{d1} are 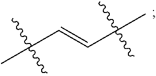 [SMILES: */C=C/*]
for each independent instance of R_{c2} and R_{d2}, R_{c2} and R_{d2} (1) independently are H or CH₃, or (2) R_{c2} and R_{d2} independently are 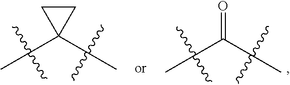
or (3) two of CR_{c2}R_{d2} are 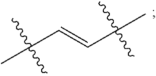 [SMILES: */C=C/*]
and * is an attachment site for a cationic dye moiety; 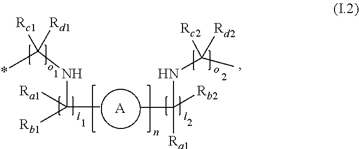
in which l₁, l₂, n, o₁, and o₂ independently are 1-4; ring A is aryl, heteroaryl, cycloalkyl, or heterocyclyl, optionally substituted with halo or C1-C6 linear or branched alkyl; for each independent instance of Rₐ₁ and R_{b1}, Rₐ₁ and R_{b1} (1) independently are H or CH₃, or (2) Rₐ₁ and R_{b1} independently are 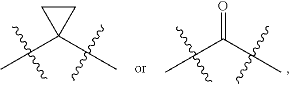
or (3) two of CRₐ₁R_{b1} are 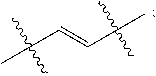 [SMILES: */C=C/*]
for each independent instance of Rₐ₂ and R_{b2}, Rₐ₂ and R_{b2} (1) independently are H or CH₃, or (2) Rₐ₂ and R_{b2} independently are 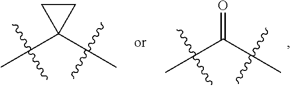
or (3) two of CRₐ₂R_{b2} are 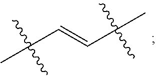 [SMILES: */C=C/*]
for each independent instance of R_{c1} and R_{d1}, R_{c1} and Rd₁ (1) independently are H or CH₃, or (2) R_{c1} and R_{d1} independently are 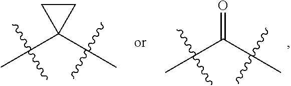
or (3) two of CR_{c1}R_{d1} are 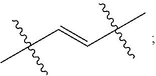 [SMILES: */C=C/*]
for each independent instance of R_{c2} and R_{d2}, R_{c2} and R_{d2} (1) independently are H or CH₃, or (2) R_{c2} and R_{d2} independently are 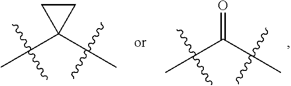
or (3) two of CR_{c2}R_{d2} are 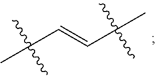 [SMILES: */C=C/*]
and * is an attachment site for a cationic dye moiety; 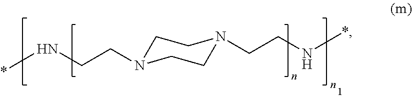
in which n is 0-6, n₁ is 1-4, and each * is an attachment site for a cationic dye moiety; 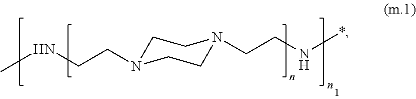
in which n is 0-6, n₁ is 1-4, and * is an attachment site for a cationic dye moiety; 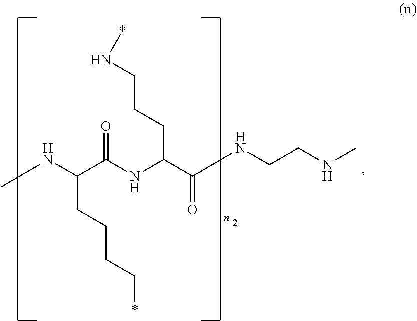
in which n₂ is 1-5 and each * is an attachment site for a cationic dye moiety; 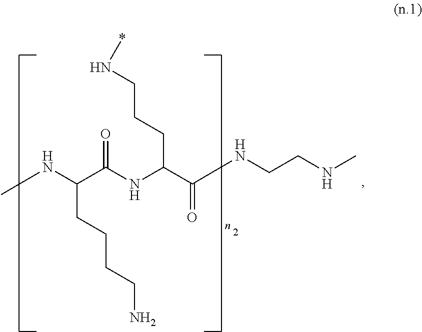
in which n₂ is 1-5 and * is an attachment site for a cationic dye moiety; 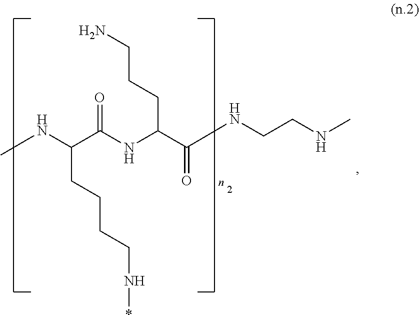
in which n₂ is 1-5 and * is an attachment site for a cationic dye moiety; 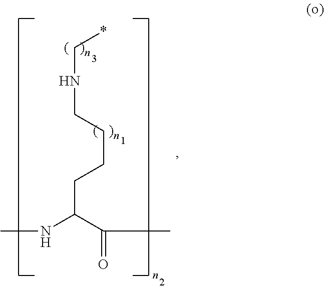
in which in which n₁ is 0-5, n₂ is 1-5, n3 is 0-5, and * is an attachment site for a cationic dye moiety; 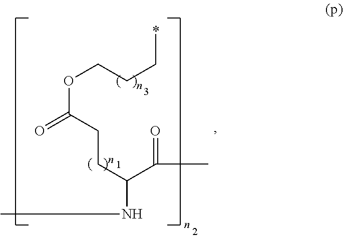
in which n₁ is 0-5, n₂ is 1-5, n3 is 0-5, and * is an attachment site for a cationic dye moiety; 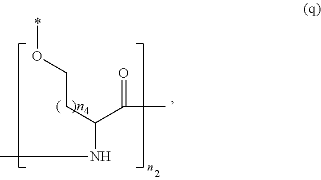
in which n₄ is 0-5, n₂ is 1-5, and * is an attachment site for a cationic dye moiety; 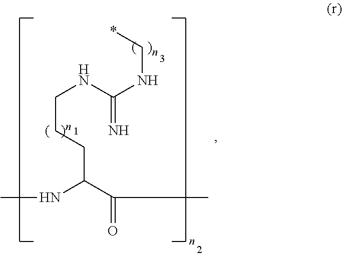
in which n₁ is 0-5, n₂ is 1-5, n3 is 0-5, and * is an attachment site for a cationic dye moiety; and 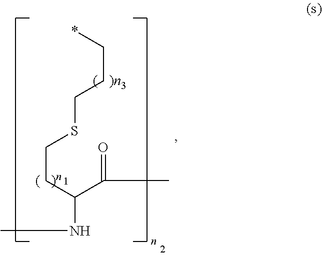
in which n₁ is 0-5, n₂ is 1-5, n₃ is 0-5 and * is an attachment site for a cationic dye moiety.

20-55. (canceled)

56. A pharmaceutical composition comprising:
(a) a cationic dye moiety bound to one or more therapeutic agents, wherein the one or more therapeutic agents independently are selected from the group consisting of a nucleic acid, a protein, and a cell;
(b) a cationic dye moiety bound to one or more therapeutic agents, wherein the one or more therapeutic agents independently are selected from the group consisting of a nucleic acid, a protein, and a cell, and wherein the cationic dye moiety is selected from the group consisting of safranin-O, toluidine blue, azure A, azure B, azure C, acridine orange, acriflavine, and methylene blue;
(c) safranin-O bound to one or more therapeutic agents independently selected from the group consisting of a small molecule, a nucleic acid, a protein, and a cell; or
(d) a cationic dye multimer of claim 11.

57. A population of repair cells to which are bound one or more cationic dye multimers of claim 11.

58. The population of claim 57, wherein the repair cells are:
(a) mesenchymal stem cells (MSCs);
(b) chondrocytes;
(c) in an aqueous suspension; or
(d) in a sheet.

59-61. (canceled)

62. A method of repairing an injured joint, comprising
(a) depositing in the injured joint the population of repair cells to which are bound one or more cationic dye multimers of claim 11; or
(b) depositing in the injured joint a population of repair cells, wherein cartilage at the articular injury site comprises a cationic dye multimer of claim 11.

63. The method of claim 62, further comprising visualizing the population by means of detecting the cationic dye multimer.

64-65. (canceled)

66. The method of claim 62, wherein the repair cells are MSCs.

67. The method of claim 62, wherein the repair cells are chondrocytes.

68. The method of claim 62, wherein the repair cells are in an aqueous suspension.

69. The method of claim 62, wherein the repair cells are in a sheet.

70. The method of claim 62, further comprising incubating the population of repair cells with a differentiation medium prior to depositing the population in the injured joint.

71. The method of claim 70, wherein the repair cells are MSCs and wherein the differentiation medium comprises TGFβ.

72. A cationic dye selected from safranin-O, toluidine blue, azure A, azure B, and azure C, which:
(a) comprises a linker; or
(b) which comprises a linker, wherein the linker is selected from the group consisting of liners (a), (a.1), (a.2), (b), (b.1), (c), (c.1), (c.2), (d), (e), (e.1), (f), (f.1), (f.2), (g), (g.1), (g.2), (h), (h.1), (h.2), (i), (i.1), (i.2), (j), (j.1), (j.2), (k), (l), (l.1), (l.2), (m), (m.1), (n), (n.1), (n.2), (o), (p), (q), (r), and (s), as defined in claim 19.

73-74. (canceled)
